(19) United States
(12) Patent Application Publication
(10) Pub. No.: US 2023/0332104 A1
(43) Pub. Date: Oct. 19, 2023

(54) ZBTB32 INHIBITORS AND USES THEREOF

(71) Applicant: NOVARTIS AG, Basel (CH)

(72) Inventors: Maria ESTEVEZ SILVA, Cambridge, MA (US); Ye GAN, Cambridge, MA (US); Yufei XU, Cambridge, MA (US); Elena ORLANDO, Somerville, MA (US)

(21) Appl. No.: 18/009,568

(22) PCT Filed: Jun. 11, 2021
(86) PCT No.: PCT/US2021/037048

§ 371 Date: Dec. 9, 2022

Related U.S. Application Data
(60) Provisional application No. 63/037,826, filed on Jun. 11, 2020.

(51) Int. Cl. C12N 5/0783; C12N 9/22; C12N 15/113; C07K 16/40; A61P 37/04

(52) U.S. Cl. CPC C12N 5/0636; C12N 9/22; C12N 15/113; C07K 16/40; A61P 37/04; C12N 2310/20; C12N 2310/11; C07K 2317/569; C12N 2501/515

(57) ABSTRACT

The disclosure provides compositions and methods for treating diseases such as cancer. The disclosure also relates to methods of making improved CART cell therapies, e.g., with reduced expression and/or a reduced biological activity of ZBTB32. The disclosure further provides ZBTB32 inhibitors, and methods of using the same alone or in combination with CART cell therapies.

### RELATED APPLICATIONS

This application is a U.S. National Phase Application under 35 U.S.C. § 371 of International Application No. PCT/US2021/037048, filed Jun. 11, 2021, which claims priority to U.S. Provisional Application No. 63/037,826, filed on Jun. 11, 2020, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Apr. 13, 2023, is named N2067-7174US_SL.txt and is 1,388,395 bytes in size.

### FIELD OF THE DISCLOSURE

The present disclosure relates generally to ZBTB32 inhibitors and their uses for treating cancer.

### BACKGROUND

Recent developments using chimeric antigen receptor (CAR) modified T cell (CART) therapy, which relies on redirecting T cells to a suitable cell-surface molecule on cancer cells, show promising results in harnessing the power of the immune system to treat cancers (see, e.g., Sadelain et al., Cancer Discovery 3:388-398 (2013)). Given the ongoing need for improved strategies for targeting diseases such as cancer, new compositions and methods for treating cancer, including improving CART therapies, are highly desirable.

### SUMMARY

Provided herein, inter alia, are CAR-expressing cells having reduced expression and/or a reduced biological activity of ZBTB32, methods of making the CAR-expressing cells and methods of using the CAR-expressing cells for treating a subject having a disease or disorder described herein. Also disclosed herein are nucleic acids, vectors, and compositions comprising CAR-expressing cells having reduced expression and/or a reduced biological activity of ZBTB32. In addition, the disclosure provides, inter alia, methods of treating cancer, methods of increasing the efficacy of other therapeutic agents or modalities, and methods of increasing immune responses, using ZBTB32 inhibitors.

CAR-Expressing Cells with Reduced ZBTB32 and Methods Relating Thereto

In an aspect, provided herein is a cell (e.g., a population of cells), e.g., an immune effector cell, expressing a chimeric antigen receptor (CAR), wherein the CAR comprises an antigen-binding domain, a transmembrane domain, and an intracellular signaling domain, and wherein the cell has reduced expression and/or a reduced biological activity of ZBTB32.

In an embodiment, the cell has no detectable expression and/or biological activity of ZBTB32.

In an embodiment, the cell comprises a ZBTB32 inhibitor, or the cell has been contacted with, or is being contacted with, a ZBTB32 inhibitor.

In an embodiment, the ZBTB32 inhibitor comprises a small molecule.

In an embodiment, the ZBTB32 inhibitor comprises: (1) a gene editing system targeting the ZBTB32 gene or one or more components thereof; (2) a nucleic acid encoding one or more components of the gene editing system; or (3) a combination of (1) and (2). In an embodiment, the ZBTB32 inhibitor comprises: (1) a gene editing system targeting the ZBTB32 gene or one or more components thereof. In an embodiment, the ZBTB32 inhibitor comprises (2) a nucleic acid encoding one or more components of the gene editing system. In an embodiment, the ZBTB32 inhibitor comprises a combination of (1) and (2).

In another aspect, disclosed herein is a method of increasing the therapeutic efficacy of a CAR-expressing cell, e.g., a cell of any of the preceding claims, e.g., a CAR19-expressing cell (e.g., CTL019 or CTL119), comprising: reducing the expression and/or a biological activity of ZBTB32 in the cell, thereby increasing the therapeutic efficacy of the CAR-expressing cell.

In yet another aspect, provided herein is method of increasing the therapeutic efficacy of a CAR-expressing cell, e.g., a cell of any of the preceding claims, e.g., a CAR19-expressing cell (e.g., CTL019 or CTL119), comprising: contacting the cell with a ZBTB32 inhibitor, e.g., a ZBTB32 inhibitor described herein, thereby increasing the therapeutic efficacy of the CAR-expressing cell.

In an embodiment, the inhibitor is: (a) a small molecule that reduces the expression and/or a biological activity of ZBTB32; (b) a gene editing system targeting the ZBTB32 gene; (c) a nucleic acid (e.g., an siRNA, shRNA, or ASO) that inhibits expression of ZBTB32; (d) a protein (e.g., a dominant negative) encoded by the ZBTB32 gene, or a binding partner of a protein encoded by the ZBTB32 gene; (e) an antibody molecule (e.g., a single-domain antibody (sdAb) or nanobody) that binds to a protein encoded by the ZBTB32 gene; (f) a nucleic acid encoding (b) or a component thereof or any of (c)-(d); or (g) any combination of (a)-(f).

In an embodiment, the cell is contacted with the ZBTB32 inhibitor ex vivo.

In an embodiment, the cell is contacted with the ZBTB32 inhibitor in vivo.

In an embodiment, the cell is contacted with the ZBTB32 inhibitor in vivo prior to delivery of a nucleic acid encoding a CAR into the cell.

In an embodiment, the cell is contacted with the ZBTB32 inhibitor in vivo after the cells have been administered to a subject in need thereof.

In an embodiment, the method further comprises contacting the cell with a Tet2 inhibitor.

In an embodiment, the method further comprises contacting the cell with an IKZF2 inhibitor.

In an embodiment, the cell has been contacted with a Tet2 inhibitor.

In an embodiment, the cell has been contacted with an IKZF2 inhibitor, e.g., an IKZF2 inhibitor described herein.

In one aspect, provided herein is a method for treating a cancer in a subject, the method comprising administering to the subject an effective amount of a CAR-expressing cell described herein.

In a related aspect, the disclosure provides a CAR-expressing cell described herein for use in treating a cancer in a subject.

In another aspect, provided herein is a CAR-expressing cell therapy for use in treating a subject in need thereof, wherein the CAR-expressing cell therapy is used in combination with a ZBTB32 inhibitor, e.g., a ZBTB32 inhibitor described herein.

In an embodiment, the subject receives a pre-treatment of the ZBTB32 inhibitor, prior to the initiation of the CAR-expressing cell therapy.

In an embodiment, the subject receives concurrent treatment with the ZBTB32 inhibitor and the CAR expressing cell therapy.

In an embodiment, the subject receives treatment with the ZBTB32 inhibitor post-CAR-expressing cell therapy.

In an embodiment, the subject has a disease associated with expression of a tumor antigen (e.g., a tumor antigen described herein), e.g., a proliferative disease, a precancerous condition, a cancer, and a non-cancer related indication associated with expression of the tumor antigen.

In an embodiment, the use further comprises determining the expression and/or a biological activity of ZBTB32 in the cell.

In an aspect, provided herein is a method of treating a subject, the method comprising: administering to the subject an effective amount of a ZBTB32 inhibitor, e.g., a ZBTB32 inhibitor described herein, thereby treating the subject, wherein the subject has received, is receiving, or is about to receive therapy comprising a CAR-expressing cell.

In a related aspect, the disclosure provides a ZBTB32 inhibitor, e.g., a ZBTB32 inhibitor described herein, for use in the treatment of a subject, wherein the subject has received, is receiving, or is about to receive therapy comprising a CAR-expressing cell.

In yet another aspect, provided herein is a method of manufacturing a CAR-expressing cell, comprising: introducing a nucleic acid encoding a CAR into a cell such that said nucleic acid (or CAR-encoding portion thereof) integrates into the genome of the cell, such that the expression and/or a biological activity of ZBTB32 is reduced, thereby manufacturing the CAR-expressing cell.

In an embodiment, the nucleic acid integrates within the ZBTB32 gene (e.g., within an intron or exon of the ZBTB32 gene).

In an embodiment, the nucleic acid integrates within a gene other than the ZBTB32 gene (e.g., within an intron or exon of the other gene).

In an embodiment, the CAR-expressing cell is manufactured according to a method of manufacture or production of a CAR-expressing cell, e.g., as described herein.

In one aspect, provided herein is a method of manufacturing a CAR-expressing cell, comprising:

contacting a CAR-expressing cell ex vivo with a ZBTB32 inhibitor, e.g., a ZBTB32 inhibitor described herein, thereby manufacturing the CAR-expressing cell.

In an embodiment, the CAR-expressing cell has an improved property, e.g., an improved property described herein, compared to the same CAR-expressing cell that has not been contacted ex vivo with the ZBTB32 inhibitor.

In an embodiment, the improved property comprises an enhanced T cell-mediated anti-tumor response, an increased proliferation and/or cytokine production, a reduced T cell exhaustion, an enhanced resistance to exhaustion and enhanced long-term immune protection in vivo, an increased expression of MHCII and/or MHCII transactivator CIITA, a higher expansion rate in vivo, an improved immunological memory phenotype, or any combination thereof.

In an embodiment, the CAR-expressing cell is manufactured according to a method of manufacture or production of a CAR-expressing cell, e.g., as described herein.

In another aspect, the disclosure provides a vector comprising a nucleotide sequence encoding a CAR and a nucleotide sequence encoding a ZBTB32 inhibitor.

In an embodiment, the inhibitor is: (a) a gene editing system targeting the ZBTB32 gene; (b) a nucleic acid (e.g., an siRNA, shRNA, or ASO) that inhibits expression of ZBTB32; (c) a protein (e.g., a dominant negative) encoded by the ZBTB32 gene, or a binding partner of a protein encoded by the ZBTB32 gene; (d) an antibody molecule (e.g., a single-domain antibody (sdAb) or nanobody) that binds to a protein encoded by the ZBTB32 gene; or (e) any combination of (a)-(d).

In an embodiment, the nucleotide sequence encoding the CAR and the nucleotide sequence encoding the inhibitor are separated by a 2A site.

In another aspect, disclosed herein is a composition for ex vivo manufacture of a CAR-expressing cell, comprising a ZBTB32 inhibitor, e.g., a ZBTB32 inhibitor described herein.

In an embodiment, the inhibitor is: (a) a gene editing system targeting the ZBTB32 gene; (b) a nucleic acid (e.g., an siRNA, shRNA, or ASO) that inhibits expression of ZBTB32; (c) a protein (e.g., a dominant negative) encoded by the ZBTB32 gene, or a binding partner of a protein encoded by the ZBTB32 gene; (d) an antibody molecule (e.g., a single-domain antibody (sdAb) or nanobody) that binds to a protein encoded by the ZBTB32 gene; or (e) any combination of (a)-(d).

In an embodiment, the nucleotide sequence encoding the CAR and the nucleotide sequence encoding the inhibitor are separated by a 2A site.

In one aspect, provided herein is a population of cells comprising one or more CAR-expressing cells described herein, wherein the population of cells comprises a higher (e.g., at least 1, 2, 3, 4, 5, 6, 7, 8, 9, or 10-fold higher) percentage of cells have a phenotype or express a marker described herein (e.g., a phenotype or a marker associated with a central memory T (T_{CM}) cell or a stem memory T (T_{SCM}) cell) than a reference population of cells.

In yet another aspect, provided herein is a population of cells comprising one or more CAR-expressing cells described herein, wherein the percentage of cytokine producing cells in the population is at least 50% (e.g., at least 60%, 70%, 80%, 85%, 90%, 95%, 97%, or 99%) higher than that of a reference population of cells.

In an embodiment, the reference population of cells is a population of cells which does not comprise one or more cells in which the expression and/or a biological activity of ZBTB32 in the cell has been reduced.

In a further aspect, provided herein is a population of cells comprising one or more CAR-expressing cells described herein, wherein at least 50% (e.g., at least 60%, 70%, 80%, 85%, 90%, 95%, 97%, or 99%) of the population of cells have a phenotype or express a marker described herein (e.g., a phenotype or a marker associated with a central memory T (TCM) cell or a stem memory T (TSCM) cell).

In some embodiments of any of the CAR-expressing cells, methods or uses disclosed herein, the gene editing system is a CRISPR/Cas system, a zinc finger nuclease system, a TALEN system, or a meganuclease system. In an embodiment, gene editing system binds to a target sequence in the ZBTB32 gene. In an embodiment, the gene editing system binds to a target sequence in an early exon or intron of the ZBTB32 gene. In an embodiment, the gene editing system binds a target sequence of the ZBTB32 gene, and the target sequence is upstream of exon 4, e.g., in exon 1, exon 2, or exon 3. In an embodiment, the gene editing system binds to a target sequence in a late exon or intron of the ZBTB32 gene. In an embodiment, the gene editing system binds a target sequence that is downstream of a preantepenultimate exon, e.g., is in an antepenultimate exon, a penultimate exon, or a last exon of the ZBTB32 gene. In an embodiment, the gene editing system binds a target sequence that comprises a splice junction of the ZBTB32 gene. In an embodiment, the gene editing system binds to a target sequence in a coding region of the ZBTB32 gene. In an embodiment, the gene editing system binds to a target sequence in a non-coding region of the ZBTB32 gene. In an embodiment, the gene editing system binds to a target sequence in a regulatory element of the ZBTB32 gene. In an embodiment, the gene editing system is a CRISPR/Cas system comprising a guide RNA (gRNA) molecule comprising a targeting sequence which hybridizes to a target sequence of the ZBTB32 gene.

In some embodiments of any of the CAR-expressing cells, methods or uses disclosed herein, the ZBTB32 inhibitor comprises a small interfering RNA (siRNA) or a small hairpin RNA (shRNA) targeting the ZBTB32 gene, or a nucleic acid encoding the siRNA or shRNA. In an embodiment, the siRNA or shRNA comprises a nucleotide sequence complementary to a sequence of an mRNA of the ZBTB32 gene. In an embodiment, the ZBTB32 inhibitor comprises an antisense oligonucleotide (ASO) targeting the ZBTB32 gene, or a nucleic acid encoding the ASO. In an embodiment, the ASO comprises a nucleotide sequence complementary to a sequence of an mRNA of the ZBTB32 gene.

In some embodiments of any of the CAR-expressing cells, methods or uses disclosed herein, the ZBTB32 inhibitor comprises a protein. In an embodiment, the ZBTB32 inhibitor comprises a dominant negative variant of a protein encoded by the ZBTB32 gene, or a nucleic acid encoding the dominant negative variant. In an embodiment, the ZBTB32 inhibitor comprises a dominant negative binding partner of a protein encoded by the ZBTB32 gene, or a nucleic acid encoding the dominant negative binding partner.

In some embodiments of any of the CAR-expressing cells, methods or uses disclosed herein, the ZBTB32 inhibitor comprises an antibody molecule, e.g., a single-domain antibody (sdAb) or nanobody, which binds to a protein encoded by the ZBTB32 gene, or a nucleic acid encoding the antibody molecule.

In some embodiments of any of the CAR-expressing cells, methods or uses disclosed herein, the ZBTB32 inhibitor comprises a nucleic acid encoding a single-domain antibody (sdAb) or nanobody that binds to a protein encoded by the ZBTB32 gene.

In some embodiments of any of the CAR-expressing cells, methods or uses disclosed herein, the cell has reduced expression of ZBTB32, e.g., reduced by at least 50%, 60%, 70%, 80%, 90%, 95%, 98%, or 99%, compared to a reference cell. In an embodiment, the level of ZBTB32 protein is reduced. In an embodiment, the stability of ZBTB32 protein is reduced. In an embodiment, the level of ZBTB32 mRNA is reduced. In an embodiment, the stability of ZBTB32 mRNA is reduced.

In an embodiment, the cell has reduced ZBTB32 transcription. In an embodiment, the cell has reduced ZBTB32 translation.

In some embodiments of any of the CAR-expressing cells, methods or uses disclosed herein, the ZBTB32 genomic locus is altered (e.g., disrupted). In an embodiment, the ZBTB32 gene comprises a deletion or insertion, e.g., a deletion or insertion that disrupts the open reading frame (ORF) or a CLL super enhancer in the ZBTB32 genomic locus. In an embodiment, the ZBTB32 gene comprises an epigenomic modification, e.g., an epigenomic modification that reduces the expression of ZBTB32.

In some embodiments of any of the CAR-expressing cells, methods or uses disclosed herein, the cell has a reduced biological activity of ZBTB32, e.g., reduced by at least 50%, 60%, 70%, 80%, 90%, 95%, 98%, or 99%, compared to a reference cell. In an embodiment, a transcription repressor function of ZBTB32 is reduced. In an embodiment, the interaction between ZBTB32 and one or more binding partners is reduced. In an embodiment, the one or more binding partners comprise Fanconi anemia complementation group C (FANCC), thioredoxin interacting protein (TXNIP), Vitamin D3 upregulated protein 1 (VDUP1), Zinc finger and BTB domain-containing protein 16 (Zbtb16), Zinc-finger elbow-related proline domain protein 2 (Zpo2), GATA binding protein 3 (Gata3), GATA binding protein 2 (Gata2), or B-cell lymphoma 6 (Bcl-6).

In some embodiments of any of the CAR-expressing cells, methods or uses disclosed herein, the cell has an enhanced T cell-mediated anti-tumor response.

In some embodiments of any of the CAR-expressing cells, methods or uses disclosed herein, the cell has increased proliferation and/or cytokine production.

In some embodiments of any of the CAR-expressing cells, methods or uses disclosed herein, the cell has an altered T cell state, e.g., an altered state of a dysfunctional T cell, e.g., reduced T cell exhaustion.

In some embodiments of any of the CAR-expressing cells, methods or uses disclosed herein, the cell has enhanced resistance to exhaustion and enhanced long-term immune protection in vivo.

In some embodiments of any of the CAR-expressing cells, methods or uses disclosed herein the cell has an increased expression of MHCII and/or MHCII transactivator CIITA.

In some embodiments of any of the CAR-expressing cells, methods or uses disclosed herein, the cell expands at a higher rate in vivo compared to a reference cell.

In some embodiments of any of the CAR-expressing cells, methods or uses disclosed herein, the cell has an improved immunological memory phenotype, e.g., a B cell memory phenotype.

In some embodiments of any of the CAR-expressing cells, methods or uses disclosed herein, the cell is an immune effector cell (e.g., a population of immune effector cells).

In some embodiments of any of the CAR-expressing cells, methods or uses disclosed herein, the immune effector cell is a T cell, a B cell, or an NK cell. In an embodiment, the immune effector cell is a T cell. In an embodiment, the T cell is an alpha beta T cell (αβ T cell). In an embodiment, the T cell is a CD4+ T cell, a CD8+ T cell, or a combination thereof. In an embodiment, the T cell is a CD8+ T cell or regulator T cell (Treg), e.g., a tumor infiltrated, dysfunctional CD8+ T cell or Treg. In an embodiment, the cell is a gamma delta T cell (76 T cell). In an embodiment, the cell is a B cell. In an embodiment, the cell is an NK cell.

In some embodiments of any of the CAR-expressing cells, methods or uses disclosed herein, the cell is a chimeric antigen receptor T (CART) cell, e.g., a non-responder's manufactured CART cell.

In some embodiments of any of the CAR-expressing cells, methods or uses disclosed herein, the cell is a human cell.

In some embodiments of any of the CAR-expressing cells, methods or uses disclosed herein, the cell further has reduced expression and/or a reduced biological activity of Tet2.

In some embodiments of any of the CAR-expressing cells, methods or uses disclosed herein, the cell further has reduced expression and/or a reduced biological activity of IKZF2.

In some embodiments of any of the CAR-expressing cells, methods or uses disclosed herein, the antigen-binding domain binds to a tumor antigen is selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In an embodiment, the tumor antigen is CD19, BCMA, CD20, or CD22. In an embodiment, the tumor antigen is CD19. In an embodiment, the tumor antigen is BCMA. In an embodiment, the tumor antigen is CD20. In an embodiment, the tumor antigen is CD22.

In an embodiment, the antigen-binding domain is an antibody or antibody fragment as described in, e.g., WO2012/079000 or WO2014/153270.

In some embodiments of any of the CAR-expressing cells, methods or uses disclosed herein, the transmembrane domain comprises: an amino acid sequence having at least one, two or three modifications but not more than 20, 10 or 5 modifications of an amino acid sequence of SEQ ID NO: 12 of WO2012/079000, or a sequence with 95-99% identity to an amino acid sequence of SEQ ID NO: 12 WO2012/079000; or the sequence of SEQ ID NO: 12 WO2012/079000.

In an embodiment, the antigen binding domain is connected to the transmembrane domain by a hinge region, wherein said hinge region comprises SEQ ID NO: 2 or SEQ ID NO: 6 of WO2012/079000, or a sequence with 95-99% identity thereof.

In some embodiments of any of the CAR-expressing cells, methods or uses disclosed herein, the intracellular signaling domain comprises a primary signaling domain and/or a costimulatory signaling domain, wherein the primary signaling domain comprises a functional signaling domain of a protein chosen from CD3 zeta, CD3 gamma, CD3 delta, CD3 epsilon, common FcR gamma (FCER1G), FcR beta (Fc Epsilon Rib), CD79a, CD79b, Fc gamma Rlla, DAP10, or DAP12. In an embodiment, the primary signaling domain comprises: an amino acid sequence having at least one, two or three modifications but not more than 20, 10 or 5 modifications of an amino acid sequence of SEQ ID NO: 18 or SEQ ID NO: 20 of WO2012/079000, or a sequence with 95-99% identity to an amino acid sequence of SEQ ID NO: 18 or SEQ ID NO: 20 of WO2012/079000; or the amino acid sequence of SEQ ID NO: 18 or SEQ ID NO: 20 of WO2012/079000.

In some embodiments of any of the CAR-expressing cells, methods or uses disclosed herein, the intracellular signaling domain comprises a costimulatory signaling domain, or a primary signaling domain and a costimulatory signaling domain, wherein the costimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In an embodiment, the costimulatory signaling domain comprises an amino acid sequence having at least one, two or three modifications but not more than 20, 10 or 5 modifications of an amino acid sequence of SEQ ID NO: 14 or SEQ ID NO: 16 of WO2012/079000, or a sequence with 95-99% identity to an amino acid sequence of SEQ ID NO: 14 or SEQ ID NO: 16 of WO2012/079000. In an embodiment, the costimulatory signaling domain comprises a sequence of SEQ ID NO: 14 or SEQ ID NO: 16 of WO2012/079000. In an embodiment, the intracellular domain comprises the sequence of SEQ ID NO: 14 or SEQ ID NO: 16 of WO2012/079000, and the sequence of SEQ ID NO: 18 or SEQ ID NO: 20 of WO2012/079000, wherein the sequences comprising the intracellular signaling domain are expressed in the same frame and as a single polypeptide chain.

In some embodiments of any of the CAR-expressing cells, methods or uses disclosed herein, the CAR further comprises a leader sequence comprises the sequence of SEQ ID NO: 2 of WO2012/079000.

In some embodiments of any of the CAR-expressing cells, methods or uses disclosed herein, the cancer is a hematological cancer. In an embodiment, the cancer is a lymphoma, a myeloma, or a leukemia. In an embodiment, the cancer is chronic lymphocytic leukemia (CLL), acute myeloid leukemia (AML), acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, a malignant lymphoproliferative condition, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenström macroglobulinemia, or pre-leukemia.

In an embodiment, the cancer is a B cell malignancy, e.g., B cell lymphoma or leukemia.

In an embodiment, the cancer is a lymphoma, e.g., a non-Hodgkin's lymphoma, a diffuse large B-cell lymphoma (DLBCL), e.g., activated B-cell (ABC) DLBCL or germinal center B-cell (GCB) DLBCL. In an embodiment, the cancer is a myeloma, e.g., a multiple myeloma (MM). In an embodiment, the cancer is a leukemia, e.g., an acute lymphocytic leukemia (ALL) or a chronic lymphocytic leukemia (CLL).

In some embodiments of any of the CAR-expressing cells, methods or uses disclosed herein, the cancer is a solid tumor. In an embodiment, the solid tumor is associated with immune cell infiltration. In an embodiment, the cancer is colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, an environmentally induced cancer, or a metastatic lesion thereof.

In some embodiments of any of the CAR-expressing cells, methods or uses disclosed herein, the cancer expresses a higher level of ZBTB32, e.g., as determined by a method described herein.

In some embodiments the methods or uses disclosed herein further comprise administering to the subject a second therapeutic agent or modality, e.g., a cancer therapy described herein.

In some embodiments the methods or uses disclosed herein further comprise administering to the subject a ZBTB32 inhibitor, e.g., a ZBTB32 inhibitor described herein.

### Methods Relating to Uses of ZBTB32 Inhibitor

In an aspect, the disclosure provides a method of treating a cancer in a subject, comprising: administering to the subject an effective amount of a ZBTB32 inhibitor and a second therapeutic agent or modality, thereby threating the cancer in the subject.

In an embodiment, the ZBTB32 inhibitor is administered prior to, concurrently with, or post administration of the second therapeutic agent or modality. In an embodiment, the ZBTB32 inhibitor comprises a small molecule.

In an embodiment, the ZBTB32 inhibitor comprises: (1) a gene editing system targeting the ZBTB32 gene or one or more components thereof; (2) a nucleic acid encoding one or more components of the gene editing system; or (3) a combination of (1) and (2).

In a related aspect, provided herein is a ZBTB32 inhibitor for use in treating a cancer in a subject, wherein the ZBTB32 inhibitor is used in combination with a second therapeutic agent or modality.

In an embodiment, the ZBTB32 inhibitor is administered prior to, concurrently with, or post administration of the second therapeutic agent or modality. In an embodiment, the ZBTB32 inhibitor comprises a small molecule.

In an embodiment, the ZBTB32 inhibitor comprises: (1) a gene editing system targeting the ZBTB32 gene or one or more components thereof; (2) a nucleic acid encoding one or more components of the gene editing system; or (3) a combination of (1) and (2).

In one aspect, provided herein is method of increasing the efficacy of a therapeutic agent or modality, comprising: administering to the subject an effective amount of a ZBTB32 inhibitor, e.g., a ZBTB32 inhibitor described herein, thereby increasing the efficacy of the therapeutic agent of modality.

In an embodiment, the subject has a cancer, e.g., a cancer described herein.

In an embodiment, the therapeutic agent or modality comprises an immunotherapy or a cell therapy, e.g., an immunotherapy or a cell therapy described herein.

In a related aspect, provided herein is a ZBTB32 inhibitor, e.g., a ZBTB32 inhibitor described herein, for use in increasing the efficacy of a therapeutic agent or modality in a subject.

In an embodiment, the subject has a cancer, e.g., a cancer described herein.

In an embodiment, the therapeutic agent or modality comprises an immunotherapy or a cell therapy, e.g., an immunotherapy or a cell therapy described herein.

In one aspect, provided herein is a method of increasing an immune response in a subject, comprising: administering to the subject an effective amount of a ZBTB32 inhibitor, e.g., a ZBTB32 inhibitor described herein, thereby increasing the immune response in the subject. In an embodiment, the subject has a cancer, e.g., a cancer described herein. In an embodiment, the therapeutic agent or modality comprises an immunotherapy or a cell therapy, e.g., an immunotherapy or a cell therapy described herein.

In a related aspect, the disclosure provides ZBTB32 inhibitor, e.g., a ZBTB32 inhibitor described herein, for use in increasing an immune response in a subject. In an embodiment, the subject has a cancer, e.g., a cancer described herein. In an embodiment, the therapeutic agent or modality comprises an immunotherapy or a cell therapy, e.g., an immunotherapy or a cell therapy described herein.

In yet another aspect, provided herein is a method of inhibiting the expression and/or a biological activity of ZBTB32, comprising: contacting a cell (e.g., an immune cell) with a ZBTB32 inhibitor, e.g., a ZBTB32 inhibitor described herein; and optionally further contacting the cell with a second therapeutic agent or modality; thereby treating the cell.

In an embodiment, the ZBTB32 inhibitor is contacted with the cell in vitro, ex vivo, or in vivo.

In one aspect, the disclosure provides a gene editing system targeting the ZBTB32 gene as described herein.

In an embodiment, the system comprises a CRISPR/Cas gene editing system, a zinc finger nuclease system, a TALEN system, or a meganuclease system.

In an embodiment, the system comprises a CRISPR/Cas gene editing system.

In an embodiment, the system comprises a gRNA molecule comprising a targeting sequence specific to a sequence of the ZBTB32 gene, and a Cas9 protein; a gRNA molecule comprising a targeting sequence specific to a sequence of the ZBTB32 gene, and a nucleic acid encoding a Cas9 protein; a nucleic acid encoding a gRNA molecule comprising a targeting sequence specific to a sequence of the ZBTB32 gene, and a Cas9 protein; or a nucleic acid encoding a gRNA molecule comprising a targeting sequence specific to a sequence of the ZBTB32 gene, and a nucleic acid encoding a Cas9 protein.

In an embodiment, the system further comprises a template DNA. In an embodiment, the template DNA comprises nucleic acid sequence encoding a CAR, e.g., a CAR as described herein.

In some embodiments of any of the methods or uses disclosed herein, the gene editing system is a CRISPR/Cas system, a zinc finger nuclease system, a TALEN system, or a meganuclease system. In an embodiment, gene editing system binds to a target sequence in the ZBTB32 gene. In an embodiment, the gene editing system binds to a target sequence in an early exon or intron of the ZBTB32 gene. In an embodiment, the gene editing system binds a target sequence of the ZBTB32 gene, and the target sequence is upstream of exon 4, e.g., in exon 1, exon 2, or exon 3. In an embodiment, the gene editing system binds to a target sequence in a late exon or intron of the ZBTB32 gene. In an embodiment, the gene editing system binds a target sequence that is downstream of a preantepenultimate exon, e.g., is in an antepenultimate exon, a penultimate exon, or a last exon of the ZBTB32 gene. In an embodiment, the gene editing system binds a target sequence that comprises a splice junction of the ZBTB32 gene. In an embodiment, the gene editing system binds to a target sequence in a coding region of the ZBTB32 gene. In an embodiment, the gene editing system binds to a target sequence in a non-coding region of the ZBTB32 gene. In an embodiment, the gene editing system binds to a target sequence in a regulatory element of the ZBTB32 gene. In an embodiment, the gene editing system is a CRISPR/Cas system comprising a guide RNA (gRNA) molecule comprising a targeting sequence which hybridizes to a target sequence of the ZBTB32 gene.

In some embodiments of any of the methods or uses disclosed herein, the ZBTB32 inhibitor comprises a small interfering RNA (siRNA) or a small hairpin RNA (shRNA) targeting the ZBTB32 gene, or a nucleic acid encoding the siRNA or shRNA. In an embodiment, the siRNA or shRNA comprises a nucleotide sequence complementary to a sequence of an mRNA of the ZBTB32 gene. In an embodiment, the ZBTB32 inhibitor comprises an antisense oligonucleotide (ASO) targeting the ZBTB32 gene, or a nucleic acid encoding the ASO. In an embodiment, the ASO comprises a nucleotide sequence complementary to a sequence of an mRNA of the ZBTB32 gene.

In some embodiments of any of the methods or uses disclosed herein, the ZBTB32 inhibitor comprises a protein. In an embodiment, the ZBTB32 inhibitor comprises a dominant negative variant of a protein encoded by the ZBTB32 gene, or a nucleic acid encoding the dominant negative variant. In an embodiment, the ZBTB32 inhibitor comprises a dominant negative binding partner of a protein encoded by the ZBTB32 gene, or a nucleic acid encoding the dominant negative binding partner.

In some embodiments of any of the methods or uses disclosed herein, the ZBTB32 inhibitor comprises an antibody molecule, e.g., a single-domain antibody (sdAb) or nanobody, which binds to a protein encoded by the ZBTB32 gene, or a nucleic acid encoding the antibody molecule.

In some embodiments of any of the methods or uses disclosed herein, the ZBTB32 inhibitor comprises a nucleic acid encoding a single-domain antibody (sdAb) or nanobody that binds to a protein encoded by the ZBTB32 gene.

In some embodiments of any of the methods or uses disclosed herein, the cell has reduced expression of ZBTB32, e.g., reduced by at least 50%, 60%, 70%, 80%, 90%, 95%, 98%, or 99%, compared to a reference cell. In an embodiment, the level of ZBTB32 protein is reduced. In an embodiment, the stability of ZBTB32 protein is reduced. In an embodiment, the level of ZBTB32 mRNA is reduced. In an embodiment, the stability of ZBTB32 mRNA is reduced.

In an embodiment, the cell has reduced ZBTB32 transcription. In an embodiment, the cell has reduced ZBTB32 translation.

In some embodiments of any of the methods or uses disclosed herein, the ZBTB32 genomic locus is altered (e.g., disrupted). In an embodiment, the ZBTB32 gene comprises a deletion or insertion, e.g., a deletion or insertion that disrupts the open reading frame (ORF) or a CLL super enhancer in the ZBTB32 genomic locus. In an embodiment, the ZBTB32 gene comprises an epigenomic modification, e.g., an epigenomic modification that reduces the expression of ZBTB32.

In some embodiments of any of the methods or uses disclosed herein, the cell has a reduced biological activity of ZBTB32, e.g., reduced by at least 50%, 60%, 70%, 80%, 90%, 95%, 98%, or 99%, compared to a reference cell. In an embodiment, a transcription repressor function of ZBTB32 is reduced. In an embodiment, the interaction between ZBTB32 and one or more binding partners is reduced. In an embodiment, the one or more binding partners comprise Fanconi anemia complementation group C (FANCC), thioredoxin interacting protein (TXNIP), Vitamin D3 upregulated protein 1 (VDUP1), Zinc finger and BTB domain-containing protein 16 (Zbtb16), Zinc-finger elbow-related proline domain protein 2 (Zpo2), GATA binding protein 3 (Gata3), GATA binding protein 2 (Gata2), or B-cell lymphoma 6 (Bcl-6).

In some embodiments of any of the methods or uses disclosed herein, the cell has an enhanced T cell-mediated anti-tumor response.

In some embodiments of any of the methods or uses disclosed herein, the cell has increased proliferation and/or cytokine production.

In some embodiments of any of the methods or uses disclosed herein, the cell has an altered T cell state, e.g., an altered state of a dysfunctional T cell, e.g., reduced T cell exhaustion.

In some embodiments of any of the methods or uses disclosed herein, the cell has enhanced resistance to exhaustion and enhanced long-term immune protection in vivo.

In some embodiments of any of the CAR-expressing cells, methods or uses disclosed herein the cell has an increased expression of MHCII and/or MHCII transactivator CIITA.

In some embodiments of any of the methods or uses disclosed herein, the inhibitor results in a higher cell expansion rate in vivo.

In some embodiments of any of the methods or uses disclosed herein, the inhibitor improves an immunological memory phenotype, e.g., a B cell memory phenotype.

In some embodiments of any of the methods or uses disclosed herein, the cancer is a hematological cancer. In an embodiment, the cancer is a lymphoma, a myeloma, or a leukemia. In an embodiment, the cancer is chronic lymphocytic leukemia (CLL), acute myeloid leukemia (AML), acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, a malignant lymphoproliferative condition, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenströmmacroglobulinemia, or pre-leukemia.

In an embodiment, the cancer is a B cell malignancy, e.g., B cell lymphoma or leukemia.

In an embodiment, the cancer is a lymphoma, e.g., a non-Hodgkin's lymphoma, a diffuse large B-cell lymphoma (DLBCL), e.g., activated B-cell (ABC) DLBCL or germinal center B-cell (GCB) DLBCL. In an embodiment, the cancer is a myeloma, e.g., a multiple myeloma (MM). In an embodiment, the cancer is a leukemia, e.g., an acute lymphocytic leukemia (ALL) or a chronic lymphocytic leukemia (CLL).

In some embodiments of any of the methods or uses disclosed herein, the cancer is a solid tumor. In an embodiment, the solid tumor is associated with immune cell infiltration. In an embodiment, the cancer is colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, an environmentally induced cancer, or a metastatic lesion thereof.

In some embodiments of any of the methods or uses disclosed herein, the cancer expresses a higher level of ZBTB32, e.g., as determined by a method described herein.

In some embodiments of any of the methods or uses disclosed herein, the subject is in need of having an increased immune response.

In some embodiments the methods or uses disclosed herein further comprise identifying the subject as in need of having an increased immune response.

In some embodiments the methods or uses disclosed herein further comprise determining the expression and/or a biological activity of ZBTB32 in the cell.

In some embodiments the methods or uses disclosed herein further comprise determining a signature associated with poor CART therapy response.

In some embodiments of any of the methods or uses disclosed herein, the second therapeutic agent or modality comprises an immunotherapy.

In some embodiments of any of the methods or uses disclosed herein, the second therapeutic agent or modality comprises an immune checkpoint inhibitor, e.g., an immune checkpoint inhibitor described herein. In an embodiment, the second therapeutic agent or modality comprises a PD-1 inhibitor, a TIM-3 inhibitor, a LAG-3 inhibitor, a CTLA-4 inhibitor, a TIGIT inhibitor, a GITR agonist, a PD-L1 inhibitor, a cytokine, a chimeric antigen receptor, an estrogen receptor antagonist, a CDK4/6 inhibitor, a CXCR2 inhibitor, a CSF-1/1R binding agent, an A2Ar antagonist, an IDO inhibitor, a STING agonist, a Galectin inhibitor, a MEK inhibitor, a c-MET inhibitor, a TGF-b inhibitor, an IL-1b inhibitor or an MDM2 inhibitor.

In some embodiments of any of the methods or uses disclosed herein, the second therapeutic agent or modality comprises a cell therapy, e.g., a T cell therapy, e.g., a CAR-expressing cell therapy described herein.

In some embodiments of any of the methods or uses disclosed herein, the second therapeutic agent or modality comprises a targeted therapy.

In some embodiments of any of the methods or uses disclosed herein, the second therapeutic agent or modality comprises a chemotherapy.

In some embodiments of any of the methods or uses disclosed herein, the second therapeutic agent or modality comprises a radiation therapy.

In some embodiments of any of the methods or uses disclosed herein, the second therapeutic agent or modality comprises a surgery.

In some embodiments of any of the methods or uses disclosed herein, the second therapeutic agent or modality comprises a hormone therapy.

In some embodiments of any of the methods or uses disclosed herein, the second therapeutic agent or modality comprises an angiogenesis inhibitor.

Although methods and materials similar or equivalent to those described herein can be used in the practice or testing of the present disclosure, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Jun. 11, 2020. When one gene or protein references a plurality of sequence accession numbers, all of the sequence variants are encompassed.

In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the disclosure will be apparent from the description and drawings, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows ZBTB32 gene expression in over 900 cancer cell lines in the Cancer Cell Line Encyclopedia database. A line indicates the transcript per million (TPM) of 10.

FIGS. 2A-2C show ZBTB32 editing and CAR19 expression. FIG. 2A shows flow cytometry analysis of GFP and CAR19 surface expression at day 10. PE-conjugated anti-CD19 CAR idiotype antibody was used to stain cell surface CAR19. FIG. 2B is a gel image showing efficient cutting of ZBTB32 gRNA6 and gRNA7 edited DNA by T7E1. FIG. 2C shows NGS results confirming the editing efficiency of ZBTB32 gRNA6 and gRNA 7. “x” indicates nucleotides insertion.

FIGS. 3A-3C show antigen-dependent proliferation and cytokine expression of ZBTB32KO CART cells. FIG. 3A provides flow cytometry analysis of % GFP+ cells demonstrating antigen-dependent proliferation of wt and ZBTB32KO CART cells in vitro. FIG. 3B depicts luminescence analysis showing wt and ZBTB32KO CART cells-mediated killing of TMD8-Luc cells in vitro. FIG. 3C shows expression of pro-inflammatory cytokines with higher levels of cytokine production by ZBTB32 KO CART than wt CART cells when co-cultured with TMD8-Luc cells in vitro.

FIGS. 4A-4B show tumor kinetics for mice treated with ZBTB32 KO CART cells or wt CART cells: FIG. 4A shows the mean tumor kinetics and FIG. 4B shows the individual tumor kinetics for all groups of mice over time. GFP wt (non-CAR), wt and ZBTB32 KO CART cells were injected at the dose of 1×10⁶ CAR+ cells per mouse on day 10 (indicated by a dotted line).

FIGS. 5A-5B show tumor kinetics for mice treated with ZBTB32 KO CART cells or wt CART cells. FIG. 5A shows the mean tumor kinetics and FIG. 5B shows the individual tumor kinetics for all groups of mice over time. Wt and ZBTB32 KO CART cells were injected at the dose of 2×10⁶ CAR+ cells per mouse on day 9 (indicated by dotted lines). PBS treated tumors grow out of compliance on day 25.

FIGS. 6A-6B show Bioluminescence of TMD8-Luc tumors treated with ZBTB32 KO CART cells or wt CART cells. FIG. 6A shows Bioluminescence (p/s) of TMD8-Luc tumors overtime. Wt and ZBTB32 KO CART cells were injected at the dose of 2×1⁰6 CAR+ cells per mouse on day 9. Solid bars indicate the median of bioluminescence for each group at each time point. FIG. 6B shows images captured on day 21 post tumor implant in PBS, ZBTB32 KO CART cells and wt CART cell treated mice.

FIGS. 7A-7D show tumor volume and tumor burden in mice treated with vehicle (PBS) ZBTB32 KO CART cells or wt CART cells. FIG. 7A shows tumor volumes as mean±SEM of each group, FIG. 7B shows tumor volumes of individual mice, FIG. 7C shows the mediam bioluminescence (p/s) of each group and FIG. 7D shows bioluminescence (p/s) of individual mice over time. Wt and ZBTB32 KO CART cells were injected at the dose of 0.4×10⁶ CAR+ cells per mouse on day 9 (indicated by a dotted line). PBS treated tumors grow out of compliance on day 23. Arrowheads in panel D indicate relapsed tumors in wt CART treated mice. A star in panel D indicates one mouse treated with ZBTB32 KO CART showing stable bioluminescence signal but no tumor.

FIGS. 8A-8D depict expression of T cell transcription factors in wt CART cells and ZBTB32KO CART cells. FIGS. 8A and 8B show flow cytometry analyses of TCF7 levels in total CART and CD8 CART cells in the blood samples. FIGS. 8C and 8D show Eomes levels in total CART and CD8 CART cells in the blood samples. WT, ZBTB32KO gRNA6 and ZBTB32KO gRNA7 CART cells were injected at the dose of 1×10⁶ CAR+ cells per mouse on day 10. Blood samples were collected at 4 and 5 weeks post CART injection. Solid bars indicate the median of each group at each time point. *: P<0.05, ***: P<0.001, ****: P<0.0001 by one-way ANOVA.

FIGS. 9A-9C shows flow cytometry analysis of total CART cells (FIG. 9A), CD4+ CART cells (FIG. 9B) and CD8+ CART cell (FIG. 9C) numbers per ul of blood. WT, ZBTB32KO gRNA6 and ZBTB32KO gRNA7 CART cells were injected at the dose of 1×10⁶ CAR+ cells per mouse on day 10. Blood samples were collected at 2 and 3 weeks post CART injection. Solid bars indicate the median of each group at each time point.

FIGS. 10A-10F show flow cytometry analyses of cytokines. FIGS. 10A and 10D show IL2 positive CD4 and CD8 CART cell numbers per ul of blood. FIGS. 10B and 10E show IFNg positive CD4 and CD8 CART cell numbers per ul of blood. FIGS. 10C and 10F show TNFαpositive CD4 and CD8 CART cell numbers per ul of blood. WT, ZBTB32KO gRNA6 and ZBTB32KO gRNA7 CART cells were injected at the dose of 1×10⁶ CAR+ cells per mouse on day 10. Blood samples were collected at 3 and 5 weeks post CART injection. Solid bars indicate the median of each group at each time point.

FIGS. 11A-11D show flow cytometry analyses of cell surface PD1 (FIGS. 11A and B) and TIM3 (FIGS. 11C and 11D) levels in CART cells in the blood samples. WT, ZBTB32KO gRNA6 and ZBTB32KO gRNA7 CART cells were injected at the dose of 1×10⁶ CAR+ cells per mouse on day 10. Blood samples were collected at 3 weeks post CART injection. Solid bars indicate the median level of each group. **: P<0.01, ***: P<0.001, ****: P<0.0001 by one-way ANOVA. MFI: Median Fluorescence Intensity.

FIGS. 12A-12C depict the number of CART cells in the spleen of tumor free mice. WT, ZBTB32KO gRNA6 and ZBTB32KO gRNA7 CART cells were injected at the dose of 1×10⁶ CAR+ cells per mouse on day 10. At day 53, CART cells were isolated from spleens of 14-15 tumor free mice. 4-5 spleens in the same group were pooled and the number of total CART cells per spleen was calculated. FIG. 12A shows the total number of CART cells. FIG. 12B shows the total number of CD4+ CART cells. FIG. 12C shows the total number of CD8+ CART cells. Data are presented as Mean±SEM.

FIGS. 13A-13C depict flow cytometry analyses of TCF7 (FIG. 13A), Eomes (FIG. 13B) and TOX (FIG. 13C) levels in CART cells in the blood samples. WT and ZBTB32KO gRNA6 CART cells were injected at the dose of 0.4×10⁶ CAR+ cells per mouse on day 9. Blood samples were collected at 20 days post CART injection. *: P<0.05, **: P<0.01, ***: P<0.001 by t-test. MFI: Median Fluorescence Intensity. Solid bars indicate the median of each group.

FIGS. 14A-14F show flow cytometry analyses of cell surface PD1, TIM3 and LAG3. FIGS. 14A and 14D show PD1 levels in CART cells in the blood samples. FIGS. 14B and 14E show TIM3 levels in CART cells in the blood samples. FIGS. 14C and 14F show LAG 3 levels in CART cells in the blood samples. WT and ZBTB32KO gRNA6 CART cells were injected at the dose of 0.4×10⁶ CAR+ cells per mouse on day 9. Blood samples were collected at 20 days post CART injection. *: P<0.05, **: P<0.01, ***: P<0.001, ****: P<0.0001 by t-test. MFI: Median Fluorescence Intensity. Solid bars indicate the median of each group.

FIGS. 15A-15C shows cell growth for control and ZBTB32 KO TMD8 cells. FIG. 15A shows the in vitro proliferation of control TMD8 cells (gRNA NT) and ZBTB32 KO TMD8 cells. FIGS. 15B-15C show the in vivo tumor growth of control TMD8 cells (gRNA NT) and ZBTB32 KO TMD8 cells. Individual (FIG. 15B) and mean (FIG. 15C) tumor growth kinetics for each cohort of mice over time are shown.

### DETAILED DESCRIPTION

The disclosures herein are based, at least in part, on the discoveries of the effects of ZBTB32 inhibition on immune cells and cancer cells. Without wishing to be bound by theory, it is believed that in some embodiments, inhibition of ZBTB32 can enhance T cell-mediated anti-tumor response. In certain embodiments, inhition of ZBTB32 enhances CART cell activity, e.g., cell expansion, cytokine production, persistence, resistance to exhaustion, and anti-tumor activity in vivo. In other embodiments, inhibition of ZBTB32 reduces cancer cell growth in vitro and in vivo.

Accordingly, the disclosures herein include, but are not limited to, methods of increasing the therapeutic efficacy of CAR-expressing cells, and methods of manufacturing CAR-expressing cells, using ZBTB32 inhibitors. Related CAR-expressing cells, therapies, nucleic acids, vectors, and compositions are also disclosed. The disclosures herein also include, but are not limited to, methods of treating cancer, methods of increasing the efficacy of other therapeutic agents or modalities, and methods of increasing immune responses, using ZBTB32 inhibitors.

### Definitions

Unless defined otherwise, all technical and scientific terms used herein have the same meaning as commonly understood by one of ordinary skill in the art to which the disclosure pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein. In some embodiments, the terms “CAR” and “CAR molecule” are used interchangeably.

In some embodiments, the cytoplasmic signaling domain comprises a primary signaling domain (e.g., a primary signaling domain of CD3-zeta). In some embodiments, the cytoplasmic signaling domain further comprises one or more functional signaling domains derived from at least one costimulatory molecule as defined below. In some embodiments, the costimulatory molecule is chosen from 41BB (i.e., CD137), CD27, ICOS, and/or CD28. In some embodiments, the CAR molecule comprises a chimeric fusion protein comprising an extracellular antigen recognition domain, a transmembrane domain and an intracellular signaling domain comprising a functional signaling domain derived from a stimulatory molecule. In some embodiments, the CAR molecule comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In some embodiments, the CAR molecule comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In some embodiments, the CAR molecule comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In some embodiments, the CAR molecule comprises an optional leader sequence at the amino-terminus (N-ter) of the CAR fusion protein. In some embodiments, the CAR molecule further comprises a leader sequence at the N-terminus of the extracellular antigen recognition domain, wherein the leader sequence is optionally cleaved from the antigen recognition domain (e.g., an scFv) during cellular processing and localization of the CAR molecule to the cellular membrane.

A CAR molecule that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR molecule that comprises an antigen binding domain that targets CD19 is referred to as CD19CAR. The CAR molecule can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and FIT fragments, scFv antibody fragments, linear antibodies, single domain antibodies such as sdAb (either VL or VH), camelid VHH domains, and multi-specific molecules formed from antibody fragments such as a bivalent fragment comprising two or more, e.g., two, Fab fragments linked by a disulfide bridge at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), A1-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-(HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR composition of the disclosure comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some embodiments, the antigen binding domain of a CAR composition of the disclosure comprises an antibody fragment. In some embodiments, the CAR molecule comprises an antibody fragment that comprises an scFv.

As used herein, the term “binding domain” or “antibody molecule” (also referred to herein as “anti-target binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the disclosure in prevention of the occurrence of tumor in the first place.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival. The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present disclosure and a combination partner, are both administered to a patient simultaneously in the form of a single entity or dosage. The term “non-fixed combination” means that the active ingredients, e.g. a compound of the present disclosure and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In embodiments, the additional therapeutic agent is administered at a therapeutic or lower-than therapeutic dose. In certain embodiments, the concentration of the second therapeutic agent that is required to achieve inhibition, e.g., growth inhibition, is lower when the second therapeutic agent is administered in combination with the first therapeutic agent, than when the second therapeutic agent is administered individually. In certain embodiments, the concentration of the first therapeutic agent that is required to achieve inhibition, e.g., growth inhibition, is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule. For example, inhibition of an activity of at least 5%, 10%, 20%, 30%, 40%, 50%, 60%, 70%, 80%, 90%, 95%, 98%, 99%, or more is included by this term. Thus, inhibition need not be 100%.

The term “activation,” “activator,” or “agonist” includes an increase in a certain parameter, e.g., an activity, of a given molecule. For example, increase of an activity of at least 5%, 10%, 20%, 30%, 40%, 50%, 60%, 70%, 80%, 90%, or more, or at least 1, 2, 3, 4, 5, 6, 7, 8, 9, 10-fold, or more, is included by this term.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Preferred cancers treated by the methods described herein include multiple myeloma, Hodgkin's lymphoma or non-Hodgkin's lymphoma.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of an antigen, e.g., a tumor antigen” includes, but is not limited to, a disease associated with a cell which expresses the antigen (e.g., wild-type or mutant antigen) or condition associated with a cell which expresses the antigen (e.g., wild-type or mutant antigen) including, e.g., proliferative diseases such as a cancer or malignancy or a precancerous condition such as a myelodysplasia, a myelodysplastic syndrome or a preleukemia; or a noncancer related indication associated with a cell which expresses the antigen (e.g., wild-type or mutant antigen). For the avoidance of doubt, a disease associated with expression of the antigen may include a condition associated with a cell which does not presently express the antigen, e.g., because expression of the antigen has been downregulated, e.g., due to treatment with a molecule targeting the antigen, but which at one time expressed the antigen. In some embodiments, the disease associated with expression of an antigen, e.g., a tumor antigen is a cancer (e.g., a solid cancer or a hematological cancer), a viral infection (e.g., HIV, a fungal infection, e.g., C. neoformans), an autoimmune disease (e.g. rheumatoid arthritis, system lupus erythematosus (SLE or lupus), pemphigus vulgaris, and Sjogren's syndrome; inflammatory bowel disease, ulcerative colitis; transplant-related allospecific immunity disorders related to mucosal immunity; and unwanted immune responses towards biologics (e.g., Factor VIII) where humoral immunity is important).

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the disclosure by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR molecule of the disclosure can be replaced with other amino acid residues from the same side chain family and the altered CAR molecule can be tested using the functional assays described herein.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR molecule recapitulates the signaling of the primary TCR independently of endogenous TCR complexes. In some embodiments, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the disclosure includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR molecule of the disclosure, the intracellular signaling domain in any one or more CAR molecules of the disclosure comprises an intracellular signaling sequence, e.g., a primary signaling sequence of CD3-zeta. The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In some embodiments, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In some embodiments, the “zeta stimulatory domain” or a “CD3-zeta stimulatory domain” is the sequence provided as SEQ ID NO: 1034 or 1037 or a variant thereof (e.g., a molecule having mutations, e.g., point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (e.g., a molecule having mutations, e.g., point mutations, fragments, insertions, or deletions). In some embodiments, the “4-1BB costimulatory domain” is the sequence provided as SEQ ID NO: 1029 or a variant thereof (e.g., a molecule having mutations, e.g., point mutations, fragments, insertions, or deletions).

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a molecule comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CAR molecules of the present disclosure include CAR molecules comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In some embodiments, the flexible polypeptide linker is a Gly/Ser linker and comprises the amino acid sequence (Gly-Gly-Gly-Ser)n, where n is a positive integer equal to or greater than 1. For example, n=1, n=2, n=3, n=4, n=5 and n=6, n=7, n=8, n=9 and n=10 (SEQ ID NO: 1009). In some embodiments, the flexible polypeptide linkers include, but are not limited to, (Gly4 Ser)4 (SEQ ID NO: 1010) or (Gly4 Ser)3 (SEQ ID NO: 1011). In some embodiments, the linkers include multiple repeats of (Gly2Ser), (GlySer) or (Gly3 Ser) (SEQ ID NO: 1012). Also included within the scope of the disclosure are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the polyA is between 50 and 5000 (SEQ ID NO: 1013), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents of the disclosure). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments, the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present disclosure, “tumor antigen” or “hyperproliferative disorder antigen” or “antigen associated with a hyperproliferative disorder” refers to antigens that are common to specific hyperproliferative disorders. In certain embodiments, the hyperproliferative disorder antigens of the present disclosure are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (e.g., castrate-resistant or therapy-resistant prostate cancer, or metastatic prostate cancer), ovarian cancer, pancreatic cancer, and the like, or a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In some embodiments, the RCARX cell is a T cell, and is referred to as a RCART cell. In some embodiments, the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g., RAD001.

The term “bioequivalent” refers to an amount of an agent other than the reference compound (e.g., RAD001), required to produce an effect equivalent to the effect produced by the reference dose or reference amount of the reference compound (e.g., RAD001). In some embodiments, the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In some embodiments, the effect is alteration of the ratio of PD-1 positive/PD-1 negative T cells, as measured by cell sorting. In some embodiments, a bioequivalent amount or dose of an mTOR inhibitor is the amount or dose that achieves the same level of P70 S6 kinase inhibition as does the reference dose or reference amount of a reference compound. In some embodiments, a bioequivalent amount or dose of an mTOR inhibitor is the amount or dose that achieves the same level of alteration in the ratio of PD-1 positive/PD-1 negative T cells as does the reference dose or reference amount of a reference compound.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In some embodiments, the low, immune enhancing, dose of mTOR inhibitor results in a decrease in the number of PD-1 positive immune effector cells, e.g., T cells or NK cells, and/or an increase in the number of PD-1 negative immune effector cells, e.g., T cells or NK cells, or an increase in the ratio of PD-1 negative immune effector cells (e.g., T cells or NK cells)/PD-1 positive immune effector cells (e.g., T cells or NK cells).

In some embodiments, the low, immune enhancing, dose of mTOR inhibitor results in an increase in the number of naive T cells. In some embodiments, the low, immune enhancing, dose of mTOR inhibitor results in one or more of the following:
an increase in the expression of one or more of the following markers: CD62Lhigh, CD127high, CD27+, and BCL2, e.g., on memory T cells, e.g., memory T cell precursors;
a decrease in the expression of KLRG1, e.g., on memory T cells, e.g., memory T cell precursors; and
an increase in the number of memory T cell precursors, e.g., cells with any one or combination of the following characteristics: increased CD62Lhigh, increased CD127high, increased CD27+, decreased KLRG1, and increased BCL2;
wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or a “relapse” as used herein refers to the reappearance of a disease (e.g., cancer) or the signs and symptoms of a disease such as cancer after a period of improvement or responsiveness, e.g., after prior treatment of a therapy, e.g., cancer therapy. For example, the period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%.

In some embodiments, a “responder” of a therapy can be a subject having complete response, very good partial response, or partial response after receiving the therapy. In some embodiments, a “non-responder” of a therapy can be a subject having minor response, stable disease, or progressive disease after receiving the therapy. In some embodiments, the subject has multiple myeloma and the response of the subject to a multiple myeloma therapy is determined based on IMWG 2016 criteria, e.g., as disclosed in Kumar, et al., Lancet Oncol. 17, e328-346 (2016), hereby incorporated herein by reference in its entirety.

Ranges: throughout this disclosure, various embodiments of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

The term “cognate antigen molecule” refers to any antigen described herein. In some embodiments, it refers to an antigen bound, e.g., recognized or targeted, by a CAR polypeptide, e.g., any target CAR molecule described herein. In some embodiments, it refers to a cancer associated antigen described herein. In some embodiments, the cognate antigen molecule is a recombinant molecule.

In the groups, radicals, or moieties defined herein, the number of carbon atoms is often specified preceding the group, for example, (C₁-C₁₀)alkyl means an alkyl group or radical having 1 to 10 carbon atoms. In general, for groups comprising two or more subgroups, the last named group is the radical attachment point, for example, “alkylaryl” means a monovalent radical of the formula alkyl-aryl-, while “arylalkyl” means a monovalent radical of the formula aryl-alkyl-. Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

The term “and/or” means either “and” or “or” unless indicated otherwise.

The term “optionally substituted” means that a given chemical moiety (e.g., an alkyl group) can (but is not required to) be bonded other substituents (e.g., heteroatoms). For instance, an alkyl group that is optionally substituted can be a fully saturated alkyl chain (e.g., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkoxy, (C₁-C₆)haloalkyl, (C₁-C₆)haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆)alkyl, —C(O)NH(C₁-C₆)alkyl, —S(O)₂(C₁-C₆)alkyl, —S(O)NH(C₁-C₆)alkyl, and S(O)N((C₁-C₆)alkyl)₂. The substituents can themselves be optionally substituted. “Optionally substituted” as used herein also refers to substituted or unsubstituted whose meaning is described below.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

The term “unsubstituted” means that the specified group bears no substituents.

Unless otherwise specifically defined, “aryl” means a cyclic, aromatic hydrocarbon group having 1 to 3 aromatic rings, including monocyclic or bicyclic groups such as phenyl, biphenyl, or naphthyl. When containing two aromatic rings (bicyclic, etc.), the aromatic rings of the aryl group are optionally joined at a single point (e.g., biphenyl), or fused (e.g., naphthyl). The aryl group is optionally substituted by one or more substituents, e.g., 1 to 5 substituents, at any point of attachment. Exemplary substituents include, but are not limited to, —H, -halogen, —CN, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆) alkyl, NH₂, NH((C₁-C₆)alkyl), N((C₁-C₆)alkyl)₂, —S(O)₂—(C₁-C₆)alkyl, —S(O)NH(C₁-C₆)alkyl, and S(O)N((C₁-C₆)alkyl)₂. The substituents are themselves optionally substituted. Furthermore, when containing two fused rings, the aryl groups optionally have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic aromatic radical of 5 to 24 ring atoms or a polycyclic aromatic radical, containing one or more ring heteroatoms selected from N, O, or S, the remaining ring atoms being C. Heteroaryl as herein defined also means a bicyclic heteroaromatic group wherein the heteroatom is selected from N, O, or S. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1A²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4 d]thiazolyl, imidazo[2,1-b][1,3,4]thiadiazolyl, thieno[2,3-b]pyrrolyl, 3H-indolyl, and derivatives thereof. Furthermore, when containing two fused rings the aryl groups herein defined may have an unsaturated or partially saturated ring fused with a fully saturated ring. Exemplary ring systems of these heteroaryl groups include indolinyl, indolinonyl, dihydrobenzothiophenyl, dihydrobenzofuran, chromanyl, thiochromanyl, tetrahydroquinolinyl, dihydrobenzothiazine,3,4-dihydro-1H-isoquinolinyl, 2,3-dihydrobenzofuran, indolinyl, indolyl, and dihydrobenzoxanyl.

Halogen or “halo” mean fluorine, chlorine, bromine, or iodine.

“Alkyl” means a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” means a straight or branched chain saturated hydrocarbon containing 1-12 carbon atoms containing a terminal “0” in the chain, e.g., —O(alkyl). Examples of alkoxy groups include, without limitation, methoxy, ethoxy, propoxy, butoxy, t-butoxy, or pentoxy groups.

“Alkenyl” means a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted and may be straight or branched.

“Alkynyl” means a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

“Alkylene” or “alkylenyl” means a divalent alkyl radical. Any of the above mentioned monovalent alkyl groups may be an alkylene by abstraction of a second hydrogen atom from the alkyl. As herein defined, alkylene may also be a (C₁-C₆)alkylene. An alkylene may further be a (C₁-C₄)alkylene. Typical alkylene groups include, but are not limited to, —CH₂—, —CH(CH₃)—, —C(CH₃)₂-, —CH₂CH₂—, —CH₂CH(CH₃)—, —CH₂C(CH₃)₂-, —CH₂CH₂CH₂—, —CH₂CH₂CH₂CH—, and the like.

“Cycloalkyl” or “carbocyclyl” means a monocyclic or polycyclic saturated or partially unsaturated carbon ring containing 3-18 carbon atoms and wherein there is not delocalized n electrons (aromaticity) shared among the ring carbons. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl and derivatives thereof. A (C₃-C₈)cycloalkyl is a cycloalkyl group containing between 3 and 8 carbon atoms. A cycloalkyl group can be fused (e.g., decalin) or bridged (e.g., norbomane).

“Heterocyclyl” or “heterocycloalkyl” means a saturated or partially saturated monocyclic or polycyclic ring containing carbon and at least one heteroatom selected from oxygen, nitrogen, or sulfur (O, N, or S) and wherein there is not delocalized n electrons (aromaticity) shared among the ring carbons or heteroatoms. The heterocycloalkyl ring structure may be substituted by one or more substituents. The substituents can themselves be optionally substituted. Examples of heterocyclyl rings include, but are not limited to, oxetanyl, azetadinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, imidazolinyl, dithiolanyl, and homotropanyl.

“Hydroxyalkyl” means an alkyl group substituted with one or more —OH groups. Examples of hydroxyalkyl groups include HO—CH₂—, HO—CH₂CH₂—, and CH₂—CH(OH)—.

“Haloalkyl” means an alkyl group substituted with one or more halogens. Examples of haloalkyl groups include, but are not limited to, trifluoromethyl, difluoromethyl, pentafluoroethyl, trichloromethyl, etc.

“Haloalkoxy” means an alkoxy group substituted with one or more halogens. Examples of haloalkyl groups include, but are not limited to, trifluoromethoxy, difluoromethoxy, pentafluoroethoxy, trichloromethoxy, etc.

“Cyano” means a substituent having a carbon atom joined to a nitrogen atom by a triple bond, e.g., C≡N.

“Amino” means a substituent containing at least one nitrogen atom (e.g., NH₂).

“Alkylamino” means an amino or NH₂ group where one of the hydrogens is replaced with an alkyl group, e.g., —NH(alkyl). Examples of alkylamino groups include, but are not limited to, methylamino (e.g., —NH(CH₃)), ethylamino, propylamino, iso-propylamino, n-butylamino, sec-butylamino, tert-butylamino, etc.

“Dialkylamino” means an amino or NH₂ group where both of the hydrogens are replaced with alkyl groups, e.g., —N(alkyl)₂. The alkyl groups on the amino group are the same or different alkyl groups. Examples of dialkylamino groups include, but are not limited to, dimethylamino (e.g., —N(CH₃)₂), diethylamino, dipropylamino, diiso-propylamino, di-n-butylamino, di-sec-butylamino, di-tert-butylamino, methyl(ethyl)amino, methyl(butylamino), etc.

“Spirocycloalkyl” or “spirocyclyl” means carbogenic bicyclic ring systems with both rings connected through a single atom. The rings can be different in size and nature, or identical in size and nature. Examples include spiropentane, spirohexane, spiroheptane, spirooctane, spirononane, or spirodecane. One or both of the rings in a spirocycle can be fused to another ring carbocyclic, heterocyclic, aromatic, or heteroaromatic ring. A (C₃-C₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

The term “ZBTB32” refers to zinc finger and BTB domain containing 32, also known as Rog, FAXF, FAZF, TZFP, ZNF538, or zinc finger and BTB domain-containing protein 32. GenBank Accession Numbers: NM_001316902.2, NM_001316903.2, and NM_014383.3 provide exemplary ZBTB32 nucleotide sequences. GenBank Accession Numbers: NP_055198.1, NP_001303831.1, and NP_001303832.1 provide exemplary ZBTB32 amino acid sequences.

The term “IL-15 receptor molecule” as used herein refers to a full-length naturally-occurring IL-15 receptor alpha (IL-15Ra) (e.g., a mammalian IL-15Ra, e.g., human IL-15Ra, e.g., GenBank Accession Number AAI21141.1), a functional fragment of IL-15Ra, or an active variant having at least 80%, 85%, 90%, 95%, 96%, 97%, 98%, or 99% sequence identity to a naturally-occurring wild type polypeptide of IL-15Ra or fragment thereof. In some embodiments, the variant is a derivative, e.g., a mutant, of a wild type polypeptide or nucleic acid encoding the same. In some embodiments, the IL-15Ra variant, e.g., active variant of IL-15Ra, has at least 50%, 60%, 70%, 80%, 85%, 90%, 95%, 96%, 97%, 98%, 99% or 100% activity of the wild type IL-15Ra polypeptide. In some embodiments, the IL-15Ra molecule comprises one or more post-translational modifications. As used herein, the terms IL-15R and IL-15Ra are interchangeable.

The term “IL-15 molecule” as used herein refers to a full-length naturally-occurring IL-15 (e.g., a mammalian IL-15, e.g., human IL-15, e.g., GenBank Accession Number AAI00963.1), a functional fragment of IL-15, or an active variant having at least 80%, 85%, 90%, 95%, 96%, 97%, 98%, or 99% sequence identity to a naturally-occurring wild type polypeptide of IL-15 or fragment thereof. In some embodiments, the variant is a derivative, e.g., a mutant, of a wild type polypeptide or nucleic acid encoding the same. In some embodiments, the IL-15 variant, e.g., active variant of IL-15, has at least 50%, 60%, 70%, 80%, 85%, 90%, 95%, 96%, 97%, 98%, 99% or 100% activity of the wild type IL-15 polypeptide. In some embodiments, the IL-15 molecule comprises one or more post-translational modifications.

As used herein, an “active variant” of a cytokine molecule refers to a cytokine variant having at least 50%, 60%, 70%, 80%, 85%, 90%, 95%, 96%, 97%, 98%, 99% or 100% activity of wild type cytokine, e.g., as measured by an art-recognized assay.

Various embodiments of the compositions and methods herein are described in further detail below. Additional definitions are set out throughout the specification.

### ZBTB32

Zinc finger and BTB domain-containing protein 32 is a protein that in humans is encoded by the ZBTB32 gene. The ZBTB32 protein is also known as fanconi anemia zinc finger protein (FAZF), testis zinc finger protein (TZFP), FANCC-Interacting Protein (FAXP), zinc finger protein 538 (ZNF538), repressor of GATA3 (ROG), and promyelocytic leukemia zinc finger and zbtb16 (PLZF)-like zinc finger protein (PLZP). It contains a N-terminal BTB/POZ domain (IPR000210) or a SKP1/BTB/POZ domain (IPR011333), and three C-terminal zinc fingers, Znf_C2H2_sf. (IPR036236), Znf_C2H2 type domain (IPR013087), a Znf_RING/FYVE/PHD domain (IPR013083), followed by a putative UBZ4 domain (Rizzo et al. (2014) Biochemistry. 53 (37): 5895-906). It is a member of the Poxviruses and Zinc-finger (POZ) and Kruppel (POK) family of proteins and was identified in multiple screens involving either immune cell tumorigenesis or immune cell development (Hoatlin et al. (1999) Blood. 94 (11): 3737-47).

The ZBTB32 protein can function as a transcriptional repressor. For example, the ZBTB32 protein can recruit histone modification enzymes to chromatin to affect gene activation (Yoon et al. Journal of Immunology. 189 (5): 2393-403). ZBTB32 can also recruit corepressors, such as nuclear receptor corepressor (NCoR) and histone deacetylases (HDACs) to its target genes, induces repressive chromatin states and acts cooperatively with other proteins, such as Blimp-1, to suppress the transcription of genes.

The ZBTB32 protein can interact with a number of proteins, include, for example, Fanconi anemia complementation group C (Fancc), thioredoxin interacting protein (Txnip), vitamin D3 upregulated protein 1 (VDUP1), zinc finger and BTB domain-containing protein 16 (Zbtb16), zinc-finger elbow-related proline domain protein 2 (Zpo2), and GATA binding protein 2 (GATA2) and GATA3 (Hoatlin et al. (1999) Blood. 94 (11): 3737-47; Tsuzuki et al. (2002) Blood. 99: 3404-3410; Miaw et al (2000) Immunity. 12: 323-333).

The ZBTB32 gene is expressed in T and B cells upon activation, but also highly expressed in testis. The expression of ZBTB32 is induced by inflammatory cytokines in natural killer cells (Beaulieu et al. (2014). Nat Immunol. 15: 546-555). ZBTB32 is highly expressed in diffuse large B-cell lymphoma (DLBCL) and appears to bind to and represses the expression of MHC class II transactivator (CIITA) and, as a consequence, MHCII genes (Yoon et al. J Immunol (2012). 189: 2393-2403). Zpo2 drives aggressive breast cancer by Zbtb32-mediated GATA3 suppression (Shahi et al. (2017). Proc Natl Acad Sci USA. 114 (12): 3169-3174). ZBTB32 is also identified in colon cancer based on a survival analysis of candidate biomarkers in a DNA methylation correlation network (Zhang et al. (2015). PLoS One. 10 (3): e0120361). The expression of Zbtb32 is upregulated after exposure to cisplatin (Sourisseau et al. (2016). Cell Cycle. 15 (2): 295-302).

In some embodiments of any of the compositions, methods or uses, disclosed herein, a ZBTB32 protein comprises an amino acid sequence having at least 80%, 85%, 90%, 95%, 96%, 97%, 98%, or 99% sequence identity to the amino acid sequence of SEQ ID NO: 2265, 2267, or 2269. In some embodiments, the ZBTB32 protein comprises the amino acid sequence of SEQ ID NO: 2265, 2267, or 2269.

In some embodiments of any of the compositions, methods, or uses, disclosed herein, the ZBTB32 protein is encoded by a nucleotide sequence having at least 80%, 85%, 90%, 95%, 96%, 97%, 98%, or 99% sequence identity to the nucleotide sequence of SEQ ID NO: 2266, 2268, or 2270. In some embodiments, the ZBTB32 protein is encoded by the nucleotide sequence of SEQ ID NO: 2266, 2268, or 2270.

In some embodiments, an immune effector cell described herein, e.g., a CAR-expressing immune effector cell, comprises a nucleic acid sequence, e.g., a transgene, comprising the sequence of SEQ ID NO: 2266, 2268, or 2270.

### Exemplary ZBTB32 Amino Acid and Nucleotide Sequences

```
Isoform 1 (Transcript Variant 1)
Amino acid: NP_055198.1
(SEQ ID NO: 2265)
      1 mslppirlps pygsdrlvql aarlrpalcd tlitvgsqef pahslvlagv sqqlgrrgqw
    61 algegispst faqllnfvyg esvelqpgel rplqeaaral gvqsleeacw rargdrakkp
  121 dpglkkhqee pekpsrnper elgdpgekqk peqvsrtggr eqemlhkhsp prgrpemaga
  181 tqeaqqeqtr skekrlqapv gqrgadgkhg vltwlrenpg gseeslrklp gplppagslq
  241 tsvtprpswa eapwlvggqp alwsillmpp rygipfyhst pttgawqevw reqriplsln
  301 apkglwsqnq lasssptpgs lpqgpaqlsp gemeesdqgh tgalatcagh edkagcpprp
  361 hpppappars rpyacsvcgk rfslkhqmet hyrvhtgekp fscslcpqrs rdfsamtkhl
  421 rthgaapyrc slcgagcpsl asmqahmrgh spsqlppgwt irstflysss rpsrpstspc
  481 cpssstt
Coding sequence: NM_014383.3
(SEQ ID NO: 2266)
      1 actcggcctt aagccgtcct gggtgtatgt gaggctgaaa gggaaggagg aacagggtct
    61 gggccgctgt gtgctctgcc gtcatctcta agaaacaacc ataccactga atgctactgt
  121 gtacttacaa accacactca tattcgtcac gtcatttcat cttcaccccc acctccaaat
  181 gaggettgaa atgagatgag atgttcctcc ctcccctttc aaccatggac ctcacactgt
  241 ggacttcctc ttagagcctc tgagttaggt acccaagcca aggcacaatg tccctgcccc
  301 ccataagact gcccagcccc tatggctctg atcggctggt acagctagca gccaggctcc
  361 ggccagcact ctgtgatact ctgatcaccg tagggagcca ggagttcccc gcccacagcc
  421 tggtgctagc aggtgtcagc cagcagctgg gccgcagggg ccagtgggct ctgggagaag
  481 gcatcagccc ttctaccttt gcccagctcc tgaactttgt gtatggggag agtgtagagc
  541 tgcagcctgg agagctaagg ccccttcagg aggcggccag ggccttggga gtgcagtccc
  601 tggaagaggc atgctggagg gctcgagggg acagggctaa aaagccagat ccaggcctga
  661 agaaacatca ggaggagcca gagaaaccct caaggaatcc tgagagagaa ctgggggacc
  721 ctggagagaa gcagaaacca gaacaggttt ctagaactgg tgggagagaa caggagatgt
  781 tgcacaagca ctcgccacca agaggcagac ccgagatggc aggagcaacg caggaggctc
  841 agcaggaaca gaccaggtca aaggagaaac gcctccaagc ccctgttggc caaaggggag
  901 cagatgggaa gcatggagtg ctcacgtggt tgagggaaaa tccagggggc tctgaggaaa
  961 gtctgcgcaa gctccctggc ccccttcccc cagcaggctc cctgcaaacc agcgtcaccc
1021 ctaggccctc gtgggctgag gccccttggt tggtgggggg ccagcctgcc ctgtggagca
1081 tcctgctgat gccgcccaga tatggcattc ccttctacca tagcaccccc accactggag
1141 cctggcagga ggtctggcgg gaacagagga tcccactgtc cctaaatgcc cccaaagggc
1201 tctggagcca gaaccagttg gcctcctcca gccctacccc aggttccctc ccccagggcc
1261 ccgcacagct cagccctggg gagatggaag agtctgatca ggggcacaca ggcgcacttg
1321 caacctgtgc gggtcatgag gacaaggcag getgcccacc tcgcccgcac cctcccccgg
1381 cccctcctgc tcggtctcgg ccctatgcgt gctctgtctg tggaaagagg ttttcactca
1441 agcatcagat ggagacgcac taccgagtcc acacaggaga gaagcccttc tectgtagcc
1501 tttgtcctca gcgctcccgg gacttctcgg ccatgaccaa gcacctgcgg acacacgggg
1561 ccgctccgta ccgctgctcc ctgtgcgggg ccggctgtcc cagcctggcc tccatgcagg
1621 cgcacatgcg cggtcactcg cccagccaac tcccgcccgg atggaccatc cgctccacct
1681 tcctctactc ctcctcgagg ccgtctcggc cctcgacctc tccctgttgt ccttcttcct
1741 ccaccacctg acggggtgtc ggtagcgtct tagccaagag tccaattaaa gaacgaaaag
1801 cgggccggct cggcttctga cctggcaccg ctgctacggc ggcctagcaa attccgcccc
1861 agaagcgccc caggaagggc gccgagtgcc ctctcctgga cgatcgcggg tcgcagaagc
1921 ccaggccagc gagccctaca gagtgaggac actgaagtgt gcaggagcga aggttaacag
1981 taggggagat tgtcgatctc atcaccataa taaagagttt cctgtgccct cccttca
Isoform 2 (Transcript Variant 2)
Amino acid: NP_001303831.1
(SEQ ID NO: 2267)
      1 mstpttgawq evwreqripl slnapkglws qnqlassspt pgstpqgpaq lspgemeesd
    61 qghtgalatc aghedkagcp prphpppapp arsrpyacsv cgkrfslkhq methyrvhtg
  121 ekpfscslcp qrsrdfsamt khlrthgaap yrcslcgagc pslasmqahm rghspsqlpp
  181 gwtirstfly sssrpsrpst spccpssstt
Coding sequence: NM_001316902.2
(SEQ ID NO: 2268)
      1 actcggcctt aagccgtcct gggtgtatgt gaggctgaaa gggaaggagg aacagggtct
    61 gggccgctgt gtgctctgcc gtcatctcta agaaacaacc ataccactga atgctactgt
  121 gtacttacaa accacactca tattcgtcac gtcatttcat cttcaccccc acctccaaat
  181 gagcaccccc accactggag cctggcagga ggtctggcgg gaacagagga tcccactgtc
  241 cctaaatgcc cccaaagggc tctggagcca gaaccagttg gcctcctcca gccctacccc
  301 aggttccctc ccccagggcc ccgcacagct cagccctggg gagatggaag agtctgatca
  361 ggggcacaca ggcgcacttg caacctgtgc gggtcatgag gacaaggcag gctgcccacc
  421 tcgcccgcac cctcccccgg cccctcctgc tcggtctcgg ccctatgcgt gctctgtctg
  481 tggaaagagg ttttcactca agcatcagat ggagacgcac taccgagtcc acacaggaga
  541 gaagcccttc tcctgtagcc tttgtcctca gcgctcccgg gacttctcgg ccatgaccaa
  601 gcacctgcgg acacacgggg ccgctccgta ccgctgctcc ctgtgcgggg ccggctgtcc
  661 cagcctggcc tccatgcagg cgcacatgcg cggtcactcg cccagccaac tcccgcccgg
  721 atggaccatc cgctccacct tcctctactc ctcctcgagg ccgtctcggc cctcgacctc
  781 tccctgttgt ccttcttcct ccaccacctg acggggtgtc ggtagcgtct tagccaagag
  841 tccaattaaa gaacgaaaag cgggccggct cggcttctga cctggcaccg ctgctacggc
  901 ggcctagcaa attccgcccc agaagcgccc caggaagggc gccgagtgcc ctctcctgga
  961 cgatcgcggg tcgcagaagc ccaggccagc gagccctaca gagtgaggac actgaagtgt
1021 gcaggagcga aggttaacag taggggagat tgtcgatctc atcaccataa taaagagttt
1081 cctgtgccct cccttca
Isoform 3 (Transcript Variant 3)
Amino acid: NP_001303832.1
(SEQ ID NO: 2269)
      1 meesdqghtg alatcaghed kagcpprphp ppapparsrp yacsvcgkrf slkhqmethy
    61 rvhtgekpfs cslcpqrsrd fsamtkhlrt hgaapyrcsl cgagcpslas mqahmrghsp
  121 sqlppgwtir stflysssrp srpstspccp ssstt
Coding sequence: NM_001316903.2
(SEQ ID NO: 2270)
      1 actcggcctt aagccgtcct gggtgtatgt gaggctgaaa gggaaggagg aacagggtct
    61 gggccgctgt gtgctctgcc gtcatctcta agaaacaacc ataccactga atgctactgt
  121 gtacttacaa accacactca tattcgtcac gtcatttcat cttcaccccc acctccaaat
  181 gagcaccccc accactggag cctggcagga ggtctggcgg gaacagaggt tccctccccc
  241 agggccccgc acagctcagc cctggggaga tggaagagtc tgatcagggg cacacaggcg
  301 cacttgcaac ctgtgcgggt catgaggaca aggcaggctg cccacctcgc ccgcaccctc
  361 ccccggcccc tcctgctcgg tctcggccct atgcgtgctc tgtctgtgga aagaggtttt
  421 cactcaagca tcagatggag acgcactacc gagtccacac aggagagaag cccttctcct
  481 gtagcctttg tcctcagcgc tcccgggact tctcggccat gaccaagcac ctgcggacac
  541 acggggccgc tccgtaccgc tgctccctgt gcggggccgg ctgtcccagc ctggcctcca
  601 tgcaggcgca catgcgcggt cactcgccca gccaactccc gcccggatgg accatccgct
  661 ccaccttcct ctactcctcc tcgaggccgt ctcggccctc gacctctccc tgttgtcctt
  721 cttcctccac cacctgacgg ggtgtcggta gcgtcttagc caagagtcca attaaagaac
  781 gaaaagcggg ccggctcggc ttctgacctg gcaccgctgc tacggcggcc tagcaaattc
  841 cgccccagaa gcgccccagg aagggcgccg agtgccctct cctggacgat cgcgggtcgc
  901 agaagcccag gccagcgagc cctacagagt gaggacactg aagtgtgcag gagcgaaggt
  961 taacagtagg ggagattgtc gatctcatca ccataataaa gagtttcctg tgccctccct
1021 tca
```

### Gene Editing Systems

According to the present disclosure, gene editing systems can be used as ZBTB32 inhibitors. Also contemplated by the present disclosure are the uses of nucleic acid encoding one or more components of a gene editing system targeting the ZBTB32 gene.

### CRISPR/Cas9 Gene Editing Systems

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR/Cas system has been modified for use in gene editing (silencing, enhancing or changing specific genes) in eukaryotes such as mice or primates. Wiedenheft et al. (2012) Nature 482: 331-8. This is accomplished by, for example, introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in an exemplary CRISPR/Cas system targeting the ZBTB32 gene, the spacers are derived from the ZBTB32 gene sequence, or a sequence of its regulatory elements.

RNA from the CRISPR locus is constitutively expressed and processed into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stem et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

The CRISPR/Cas system can thus be used to modify, e.g., delete one or more nucleic acids, the ZBTB32 gene, or a gene regulatory element of the ZBTB32 gene, or introduce a premature stop which thus decreases expression of a functional of the ZBTB32 gene. The CRISPR/Cas system can alternatively be used like RNA interference, turning off the ZBTB32 gene in a reversible fashion. In a mammalian cell, for example, the RNA can guide the Cas protein to a promoter of the ZBTB32 gene, sterically blocking RNA polymerases.

CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of binding to a Cas, e.g., Cas9 enzyme, and (2) a Cas, e.g., Cas9, protein. The targeting sequence and the sequence which is capable of binding to a Cas, e.g., Cas9 enzyme, may be disposed on the same or different molecules. If disposed on different molecules, each includes a hybridization domain which allows the molecules to associate, e.g., through hybridization.

An exemplary gRNA molecule of the present disclosure comprises, e.g., consists of a first nucleic acid having the sequence (where the “n” 's refer to the residues of the targeting sequence (e.g., as described herein, e.g., in Table 3), and may consist of 15-25 nucleotides, e.g., consist of 20 nucleotides):

(SEQ ID NO: 3022)
nnnnnnnnnnnnnnnnnnnnGUUUUAGAGCUAUGCUGUUUUG;

and a second nucleic acid sequence having the sequence:

AACUUACCAAGGAACAGCAUAGCAAGUUAAAAUAAGGCUAGUCCGUUAUCAAC UUGAAAAAGUGGCACCGAGUCGGUGC, optionally with 1, 2, 3, 4, 5, 6, or 7 (e.g., 4 or 7, e.g., 7) additional U nucleotides at the 3′ end (SEQ ID NO: 3024).

The second nucleic acid molecule may alternatively consist of a fragment of the sequence above, wherein such fragment is capable of hybridizing to the first nucleic acid. An example of such second nucleic acid molecule is:

AACAGCAUAGCAAGUUAAAAUAAGGCUAGUCCGUUAUCAACUUGAAAAAGUG GCACCGAGUCGGUGC, optionally with 1, 2, 3, 4, 5, 6, or 7 (e.g., 4 or 7, e.g., 7) additional U nucleotides at the 3′ end (SEQ ID NO: 3026).

Another exemplary gRNA molecule of the present disclosure comprises, e.g., consists of a first nucleic acid having the sequence (where the “n” 's refer to the residues of the targeting sequence (e.g., as described herein, e.g., in Table 3), and may consist of 15-25 nucleotides, e.g., consist of 20 nucleotides):

nnnnnnnnnnnnnnnnnnnGUUUUAGAGCUAGAAAUAGCAAGUUAAAAUAAGGCUAGU CCGUUAUCAACUUGAAAAAGUGGCACCGAGUCGGUGC (SEQ ID NO: 3028), optionally with 1, 2, 3, 4, 5, 6, or 7 (e.g., 4 or 7, e.g., 4) additional U nucleotides at the 3′ end. Artificial CRISPR/Cas systems can be generated which inhibit the ZBTB32 gene, using technology known in the art, e.g., that are described in U.S. Publication No. 20140068797, WO2015/048577, and Cong (2013) Science 339: 819-823. Other artificial CRISPR/Cas systems that are known in the art may also be generated which inhibit the ZBTB32 gene, e.g., that described in Tsai (2014) Nature Biotechnol., 32:6 569-576, U.S. Pat. Nos. 8,871,445; 8,865,406; 8,795,965; 8,771,945; and 8,697,359, the contents of which are hereby incorporated by reference in their entirety. Such systems can be generated which inhibit the ZBTB32 gene, by, for example, engineering a CRISPR/Cas system to include a gRNA molecule comprising a targeting sequence that hybridizes to a sequence of a target gene, e.g., the ZBTB32 gene. In embodiments, the gRNA comprises a targeting sequence which is fully complementarity to 15-25 nucleotides, e.g., 20 nucleotides, of a target gene, e.g., the ZBTB32 gene. In embodiments, the 15-25 nucleotides, e.g., 20 nucleotides, of a target gene, e.g., the ZBTB32 gene, are disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C).

In one embodiment, foreign DNA can be introduced into the cell along with the CRISPR/Cas system, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the CRISPR/Cas system. As shown herein, in the examples, but without being bound by theory, such integration may lead to the expression of the CAR as well as disruption of the ZBTB32 gene. Such foreign DNA molecule is referred to herein as “template DNA.” In embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

In an embodiment, the CRISPR/Cas system of the present disclosure comprises Cas9, e.g., S. pyogenes Cas9, and a gRNA comprising a targeting sequence which hybridizes to a sequence of the ZBTB32 gene. In an embodiment, the CRISPR/Cas system comprises nucleic acid encoding a gRNA specific for the ZBTB32 gene, and a nucleic acid encoding a Cas protein, e.g., Cas9, e.g., S. pyogenes Cas9. In an embodiment, the CRISPR/Cas system comprises a gRNA specific for the ZBTB32 gene, and a nucleic acid encoding a Cas protein, e.g., Cas9, e.g., S. pyogenes Cas9.

### TALEN Gene Editing Systems

TALENs are produced artificially by fusing a TAL effector DNA binding domain to a DNA cleavage domain. Transcription activator-like effects (TALEs) can be engineered to bind any desired DNA sequence, including a portion of the HLA or TCR gene. By combining an engineered TALE with a DNA cleavage domain, a restriction enzyme can be produced which is specific to any desired DNA sequence, including a HLA or TCR sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A TALEN specific for the ZBTB32 gene, can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the TALEN. As shown herein, in the examples, but without being bound by theory, such integration may lead to the expression of the CAR as well as disruption of the ZBTB32 gene. Such foreign DNA molecule is referred to herein as “template DNA.” In embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

TALENs specific to sequences in the ZBTB32 gene, can be constructed using any method known in the art, including various schemes using modular components. Zhang et al. (2011) Nature Biotech. 29: 149-53; Geibler et al. (2011) PLoS ONE 6: e19509; U.S. Pat. Nos. 8,420,782; 8,470,973, the contents of which are hereby incorporated by reference in their entirety.

### Zinc Finger Nucleases

“ZFN” or “Zinc Finger Nuclease” refer to a zinc finger nuclease, an artificial nuclease which can be used to modify, e.g., delete one or more nucleic acids of, a desired nucleic acid sequence, e.g., the ZBTB32 gene.

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression of the ZBTB32 gene, in a cell. ZFNs can also be used with homologous recombination to mutate the ZBTB32 gene, or to introduce nucleic acid encoding a CAR at a site at or near the targeted sequence. As discussed above, the nucleic acid encoding a CAR may be introduced as part of a template DNA. In embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

ZFNs specific to sequences in the ZBTB32 gene, can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; and Guo et al. (2010) J. Mol. Biol. 400: 96; U.S. Patent Publication 2011/0158957; and U.S. Patent Publication 2012/0060230, the contents of which are hereby incorporated by reference in their entirety. In embodiments, The ZFN gene editing system may also comprise nucleic acid encoding one or more components of the ZFN gene editing system, e.g., a ZFN gene editing system targeted to the ZBTB32 gene.

Without being bound by theory, it is believed that use of gene editing systems (e.g., CRISPR/Cas gene editing systems) which target the ZBTB32 gene, may allow one to modulate (e.g., inhibit) one or more functions of the ZBTB32 gene, by, for example, causing an editing event which results in expression of a truncated ZBTB32 gene. Again, without being bound by theory, such a truncated ZBTB32 gene product may preserve one or more functions of the ZBTB32 gene product (e.g., a scaffolding function), while inhibiting one or more other functions ofthe ZBTB32 gene product (e.g., a catalytic function), and as such, may be preferable. Gene editing systems which target a late exon or intron of the ZBTB32 gene, may be particularly preferred in this regard. In an aspect, the gene editing system of the disclosure targets a late exon or intron of the ZBTB32 gene. In an aspect, the gene editing system of the disclosure targets an exon or intron downstream of exon 8. In an aspect, the gene editing system targets exon 8 or exon 9, e.g., exon 9, of the ZBTB32 gene.

Without being bound by theory, it may also be preferable in other embodiments to target an early exon or intron of the ZBTB32 gene, for example, to introduce a premature stop codon in the targeted gene which results in no expression of the gene product, or expression of a completely non-functional gene product. Gene editing systems which target an early exon or intron of the ZBTB32 gene, may be particularly preferred in this regard. In an aspect, the gene editing system of the disclosure targets an early exon or intron of the ZBTB32 gene. In an aspect, the gene editing system of the disclosure targets an exon or intron upstream of exon 4. In embodiments, the gene editing system targets exon 1, exon 2, or exon 3, e.g., exon 3, of the ZBTB32 gene.

Without being bound by theory, it may also be preferable in other embodiments to target a sequence of the ZBTB32 gene, which is specific to one or more isoforms of the gene but does not affect one or more other isoforms of the gene. In embodiments, it may be preferable to specifically target an isoform of the ZBTB32 gene which contain a catalytic domain.

### Double-Stranded RNA, e.g., SiRNA or ShRNA, Inhibitors

According to the present disclosure, double stranded RNA (“dsRNA”), e.g., siRNA or shRNA can be used as ZBTB32 inhibitors. Also contemplated by the present disclosure are the uses of nucleic acid encoding said dsRNA inhibitors.

In an embodiment, the modulator (e.g., inhibitor) of the ZBTB32 gene is a nucleic acid, e.g., a dsRNA, e.g., a siRNA or shRNA specific for a nucleic acid encoding a ZBTB32 gene product, e.g., genomic DNA or mRNA encoding a ZBTB32 gene product.

An aspect of the disclosure provides a composition comprising a dsRNA, e.g., a siRNA or shRNA, comprising at least 15 contiguous nucleotides, e.g., 15, 16, 17, 18, 19, 20, 21, 22, 23, 24 or 25 contiguous nucleotides, e.g., 21 contiguous nucleotides, which are complementary (e.g., 100% complementary) to a sequence of the ZBTB32 gene, nucleic acid sequence (e.g., genomic DNA or mRNA encoding a ZBTB32 gene product. It is understood that some of the target sequences and/or shRNA molecules are presented as DNA, but the dsRNA agents targeting these sequences or comprising these sequences can be RNA, or any nucleotide, modified nucleotide or substitute disclosed herein and/or known in the art, provided that the molecule can still mediate RNA interference.

In an embodiment, a nucleic acid molecule that encodes a dsRNA molecule that inhibits expression of the ZBTB32 gene, is operably linked to a promoter, e.g., a H1- or a U6-derived promoter such that the dsRNA molecule that inhibits expression of the ZBTB32 gene, is expressed within a CAR-expressing cell. See e.g., Tiscornia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, NY, USA, 2007; Brummelkamp T R, et al. (2002) Science 296: 550-553; Miyagishi M, et al. (2002) Nat. Biotechnol. 19: 497-500. In an embodiment the nucleic acid molecule that encodes a dsRNA molecule that inhibits expression of the ZBTB32 gene, is present on the same vector, e.g., a lentiviral vector, that comprises a nucleic acid molecule that encodes a component, e.g., all of the components, of the CAR. In such an embodiment, the nucleic acid molecule that encodes a dsRNA molecule that inhibits expression of the ZBTB32 gene, is located on the vector, e.g., the lentiviral vector, 5′- or 3′- to the nucleic acid that encodes a component, e.g., all of the components, of the CAR. The nucleic acid molecule that encodes a dsRNA molecule that inhibits expression of the ZBTB32 gene, can be transcribed in the same or different direction as the nucleic acid that encodes a component, e.g., all of the components, of the CAR. In an embodiment the nucleic acid molecule that encodes a dsRNA molecule that inhibits expression of the ZBTB32 gene, is present on a vector other than the vector that comprises a nucleic acid molecule that encodes a component, e.g., all of the components, of the CAR. In an embodiment, the nucleic acid molecule that encodes a dsRNA molecule that inhibits expression of the ZBTB32 gene, is transiently expressed within a CAR-expressing cell. In an embodiment, the nucleic acid molecule that encodes a dsRNA molecule that inhibits expression of the ZBTB32 gene, is stably integrated into the genome of a CAR-expressing cell.

Examples of nucleic acid sequences that encode shRNA sequences are provided below. The target sequence refers to the sequence within the ZBTB32 genomic DNA (or surrounding DNA). The nucleic acid encoding ZBTB32 shRNA encodes shRNA molecules useful in the present disclosure. In embodiments, the ZBTB32 inhibitor is an siRNA or shRNA specific for a target sequence listed below, or specific for its mRNA complement.

### Antibody Molecules, e.g., Single-Domain Antibodies

According to the present disclosure, antibody molecules can be used as ZBTB32 inhibitors. Also contemplated by the present disclosure are the uses of nucleic acid encoding the antibody molecules targeting a protein encoded by the ZBTB32 gene.

In some embodiments, the ZBTB32 inhibitor is a single-domain antibody (sdAb), also known as a nanobody. In other embodiments, the ZBTB32 inhibitor is a nucleic acid encoding the single domain antibody.

Single-domain antibodies can include antibodies whose complementary determining regions are part of a single-domain polypeptide. Examples include, but are not limited to, heavy chain antibodies, antibodies naturally devoid of light chains, single-domain antibodies derived from conventional 4-chain antibodies, engineered antibodies and single-domain scaffolds other than those derived from antibodies. Single-domain antibodies may be any of the art, or any future single-domain antibodies. Single-domain antibodies may be derived from any species including, but not limited to mouse, human, camel, llama, fish, shark, goat, rabbit, and bovine. According to another aspect of the disclosure, a single-domain antibody is a naturally occurring single-domain antibody known as heavy chain antibody devoid of light chains. Such single-domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the disclosure.

### Chimeric Antigen Receptor (CAR)

In some embodiments, disclosed herein are methods of making and using an immune effector cell (e.g., a population of immune effector cells) that expresses a CAR molecule (e.g., as described herein), and has reduced expression and/or a reduced biological activity of ZBTB32. In some embodiments, an exemplary CAR construct comprises an optional leader sequence (e.g., a leader sequence described herein), an antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In some embodiments, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

Sequences of non-limiting examples of various components that can be part of a CAR molecule described herein, are listed in Table 1 and Table 2, where “aa” stands for amino acids, and “na” stands for nucleic acids that encode the corresponding peptide.

TABLE 1
Exemplary sequences for various components of CAR

| | | |
|---|---|---|
| SEQ ID | pGK | AGCTTATGGTGCCCCAACCCCAACGCGGAAAAGGTTCCGTCGGGAC |
| NO: 13 | promoter | CCAAACGCGTCCCTGCGCCGACGAGACCCGCACCAAGGCCCTTTGC |
| | | GTCGCCGCGGCTGGGACCCAGAGCGTGTAAGAAGTGCAGGCAAGC |
| | | GTCGCAGTGGGCCTAGAAGCGGCGATGGGAACACCCGGGGGGCCG |
| | | CTGCGAAGGACGAGGCGGGGATTCAGCCCTTCCAAGGAACGCCAAG |
| | | CGCCGCACGGCCTGCACTATTTGCCTTCGGCGTGCAGAGTGATCATG |
| | | GGAGCGTCTGCCTGTCGCGGTCCCTCGTTACCGTCGCGCGGCTGGCG |
| | | CTACCCGACACCGGTTATCGCCGACGAGTCGTCCCGCGCGGCTCTCG |
| | | TCGCCGGCCCTTCCCCGCCACGCCCTCCGCCCCACACCCCGCCATCA |
| | | CACCCGGGACAAGGACGGGCGCGCCACAAGGCGTAAGACGTTCGG |
| | | AGGCCTCGCGTGCAGCCGTCAGCCGAGGGAGCAACTGGCTTAGTGG |
| | | CTGGAGAGAGGGGT |
| SEQ ID | CTL019 | GACATCCAGATGACACAGACTACATCCTCCCTGTCTGCCTCTCTGGG |
| NO: 14 | scFv | AGACAGAGTCACCATCAGTTGCAGGGCAAGTCAGGACATTAGTAAA |
| | nucleotide | TATTTAAATTGGTATCAGCAGAAACCAGATGGAACTGTTAAACTCCT |
| | sequence | GATCTACCATACATCAAGATTACACTCAGGAGTCCCATCAAGGTTC |
| | | AGTGGCAGTGGGTCTGGAACAGATTATTCTCTCACCATTAGCAACCT |
| | | GGAGCAAGAAGATATTGCCACTTACTTTTGCCAACAGGGTAATACG |
| | | CTTCCGTACACGTTCGGAGGGGGGACCAAGCTGGAGATCACAGGTG |
| | | GCGGTGGCTCGGGCGGTGGTGGGTCGGGTGGCGGCGGATCTGAGGT |
| | | GAAACTGCAGGAGTCAGGACCTGGCCTGGTGGCGCCCTCACAGAGC |
| | | CTGTCCGTCACATGCACTGTCTCAGGGGTCTCATTACCCGACTATGG |
| | | TGTAAGCTGGATTCGCCAGCCTCCACGAAAGGGTCTGGAGTGGCTG |
| | | GGAGTAATATGGGGTAGTGAAACCACATACTATAATTCAGCTCTCA |
| | | AATCCAGACTGACCATCATCAAGGACAACTCCAAGAGCCAAGTTTT |
| | | CTTAAAAATGAACAGTCTGCAAACTGATGACACAGCCATTTACTAC |
| | | TGTGCCAAACATTATTACTACGGTGGTAGCTATGCTATGGACTACTG |
| | | GGGCCAAGGAACCTCAGTCACCGTCTCCTCA |
| SEQ ID | CTL019 | DIQMTQTTSSLSASLGDRVTISCRASQDISKYLNWYQQKPDGTVKLLIY |
| NO: 15 | scFv amino | HTSRLHSGVPSRFSGSGSGTDYSLTISNLEQEDIATYFCQQGNTLPYTFG |
| | acid | GGTKLEITGGGGSGGGGSGGGGSEVKLQESGPGLVAPSQSLSVTCTVS |
| | sequence | GVSLPDYGVSWIRQPPRKGLEWLGVIWGSETTYYNSALKSRLTIIKDNS |
| | | KSQVFLKMNSLQTDDTAIYYCAKHYYYGGSYAMDYWGQGTSVTVSS |
| SEQ ID | P2A | GGAAGCGGAGCTACTAACTTCAGCCTGCTGAAGCAGGCTGGAGACG |
| NO: 23 | nucleotide | TGGAGGAGAACCCTGGACCT |
| | sequence | |
| SEQ ID | P2A amino | GSGATNFSLLKQAGDVEENPGP |
| NO: 24 | acid | |
| | sequence | |
| SEQ ID | CTL019 | GACATCCAGATGACACAGACTACATCCTCCCTGTCTGCCTCTCTGGG |
| NO: 25 | full-length | AGACAGAGTCACCATCAGTTGCAGGGCAAGTCAGGACATTAGTAAA |
| | nucleotide | TATTTAAATTGGTATCAGCAGAAACCAGATGGAACTGTTAAACTCCT |
| | sequence | GATCTACCATACATCAAGATTACACTCAGGAGTCCCATCAAGGTTC |
| | | AGTGGCAGTGGGTCTGGAACAGATTATTCTCTCACCATTAGCAACCT |
| | | GGAGCAAGAAGATATTGCCACTTACTTTTGCCAACAGGGTAATACG |
| | | CTTCCGTACACGTTCGGAGGGGGGACCAAGCTGGAGATCACAGGTG |
| | | GCGGTGGCTCGGGCGGTGGTGGGTCGGGTGGCGGCGGATCTGAGGT |
| | | GAAACTGCAGGAGTCAGGACCTGGCCTGGTGGCGCCCTCACAGAGC |
| | | CTGTCCGTCACATGCACTGTCTCAGGGGTCTCATTACCCGACTATGG |
| | | TGTAAGCTGGATTCGCCAGCCTCCACGAAAGGGTCTGGAGTGGCTG |
| | | GGAGTAATATGGGGTAGTGAAACCACATACTATAATTCAGCTCTCA |
| | | AATCCAGACTGACCATCATCAAGGACAACTCCAAGAGCCAAGTTTT |
| | | CTTAAAAATGAACAGTCTGCAAACTGATGACACAGCCATTTACTAC |
| | | TGTGCCAAACATTATTACTACGGTGGTAGCTATGCTATGGACTACTG |
| | | GGGCCAAGGAACCTCAGTCACCGTCTCCTCAACCACGACGCCAGCG |
| | | CCGCGACCACCAACACCGGCGCCCACCATCGCGTCGCAGCCCCTGT |
| | | CCCTGCGCCCAGAGGCGTGCCGGCCAGCGGCGGGGGGCGCAGTGCA |
| | | CACGAGGGGGCTGGACTTCGCCTGTGATATCTACATCTGGGCGCCCT |
| | | TGGCCGGGACTTGTGGGGTCCTTCTCCTGTCACTGGTTATCACCCTT |
| | | TACTGCAAACGGGGCAGAAAGAAACTCCTGTATATATTCAAACAAC |
| | | CATTTATGAGACCAGTACAAACTACTCAAGAGGAAGATGGCTGTAG |
| | | CTGCCGATTTCCAGAAGAAGAAGAAGGAGGATGTGAACTGAGAGT |
| | | GAAGTTCAGCAGGAGCGCAGACGCCCCCGCGTACAAGCAGGGCCA |
| | | GAACCAGCTCTATAACGAGCTCAATCTAGGACGAAGAGAGGAGTAC |
| | | GATGTTTTGGACAAGAGACGTGGCCGGGACCCTGAGATGGGGGGAA |
| | | AGCCGAGAAGGAAGAACCCTCAGGAAGGCCTGTACAATGAACTGC |
| | | AGAAAGATAAGATGGCGGAGGCCTACAGTGAGATTGGGATGAAAG |
| | | GCGAGCGCCGGAGGGGCAAGGGGCACGATGGCCTTTACCAGGGTCT |
| | | CAGTACAGCCACCAAGGACACCTACGACGCCCTTCACATGCAGGCC |
| | | CTGCCCCCTCGC |
| SEQ ID | CTL019 | DIQMTQTTSSLSASLGDRVTISCRASQDISKYLNWYQQKPDGTVKLLIY |
| NO: 26 | full-length | HTSRLHSGVPSRFSGSGSGTDYSLTISNLEQEDIATYFCQQGNTLPYTFG |
| | amino acid | GGTKLEITGGGGSGGGGSGGGGSEVKLQESGPGLVAPSQSLSVTCTVS |
| | sequence | GVSLPDYGVSWIRQPPRKGLEWLGVIWGSETTYYNSALKSRLTIIKDNS |
| | | KSQVFLKMNSLQTDDTAIYYCAKHYYYGGSYAMDYWGQGTSVTVSS |
| | | TTTPAPRPPTPAPTIASQPLSLRPEACRPAAGGAVHTRGLDFACDIYIWA |
| | | PLAGTCGVLLLSLVITLYCKRGRKKLLYIFKQPFMRPVQTTQEEDGCSC |
| | | RFPEEEEGGCELRVKFSRSADAPAYKQGQNQLYNELNLGRREEYDVLD |
| | | KRRGRDPEMGGKPRRKNPQEGLYNELQKDKMAEAYSEIGMKGERRR |
| | | GKGHDGLYQGLSTATKDTYDALHMQALPPR |

TABLE 2
Exemplary sequences of various components of CAR
(aa-amino acid sequence, na-nucleic acid sequence).

| SEQ ID | | |
|---|---|---|
| NO: | description | Sequence |
| SEQ ID | EF-1 | CGTGAGGCTCCGGTGCCCGTCAGTGGGCAGAGCGCACATCGCCC |
| NO: 1014 | promoter | ACAGTCCCCGAGAAGTTGGGGGGAGGGGTCGGCAATTGAACCG |
| | | GTGCCTAGAGAAGGTGGCGCGGGGTAAACTGGGAAAGTGATGT |
| | | CGTGTACTGGCTCCGCCTTTTTCCCGAGGGTGGGGGAGAACCGT |
| | | ATATAAGTGCAGTAGTCGCCGTGAACGTTCTTTTTCGCAACGGGT |
| | | TTGCCGCCAGAACACAGGTAAGTGCCGTGTGTGGTTCCCGCGGG |
| | | CCTGGCCTCTTTACGGGTTATGGCCCTTGCGTGCCTTGAATTACT |
| | | TCCACCTGGCTGCAGTACGTGATTCTTGATCCCGAGCTTCGGGTT |
| | | GGAAGTGGGTGGGAGAGTTCGAGGCCTTGCGCTTAAGGAGCCCC |
| | | TTCGCCTCGTGCTTGAGTTGAGGCCTGGCCTGGGCGCTGGGGCC |
| | | GCCGCGTGCGAATCTGGTGGCACCTTCGCGCCTGTCTCGCTGCTT |
| | | TCGATAAGTCTCTAGCCATTTAAAATTTTTGATGACCTGCTGCGA |
| | | CGCTTTTTTTCTGGCAAGATAGTCTTGTAAATGCGGGCCAAGATC |
| | | TGCACACTGGTATTTCGGTTTTTGGGGCCGCGGGCGGCGACGGG |
| | | GCCCGTGCGTCCCAGCGCACATGTTCGGCGAGGCGGGGCCTGCG |
| | | AGCGCGGCCACCGAGAATCGGACGGGGGTAGTCTCAAGCTGGCC |
| | | GGCCTGCTCTGGTGCCTGGCCTCGCGCCGCCGTGTATCGCCCCGC |
| | | CCTGGGCGGCAAGGCTGGCCCGGTCGGCACCAGTTGCGTGAGCG |
| | | GAAAGATGGCCGCTTCCCGGCCCTGCTGCAGGGAGCTCAAAATG |
| | | GAGGACGCGGCGCTCGGGAGAGCGGGCGGGTGAGTCACCCACA |
| | | CAAAGGAAAAGGGCCTTTCCGTCCTCAGCCGTCGCTTCATGTGA |
| | | CTCCACGGAGTACCGGGCGCCGTCCAGGCACCTCGATTAGTTCT |
| | | CGAGCTTTTGGAGTACGTCGTCTTTAGGTTGGGGGGAGGGGTTTT |
| | | ATGCGATGGAGTTTCCCCACACTGAGTGGGTGGAGACTGAAGTT |
| | | AGGCCAGCTTGGCACTTGATGTAATTCTCCTTGGAATTTGCCCTT |
| | | TTTGAGTTTGGATCTTGGTTCATTCTCAAGCCTCAGACAGTGGTT |
| | | CAAAGTTTTTTTCTTCCATTTCAGGTGTCGTGA |
| SEQ ID | Leader (aa) | MALPVTALLLPLALLLHAARP |
| NO: 1015 | | |
| SEQ ID | Leader (na) | ATGGCCCTGCCTGTGACAGCCCTGCTGCTGCCTCTGGCTCTGCTG |
| NO: 1016 | | CTGCATGCCGCTAGACCC |
| SEQ ID | Leader (na) | ATGGCCCTCCCTGTCACCGCCCTGCTGCTTCCGCTGGCTCTTCTG |
| NO: 1017 | | CTCCACGCCGCTCGGCCC |
| SEQ ID | Leader (aa) | MLLLVTSLLLCELPHPAFLLIP |
| NO: 3204 | | |
| SEQ ID | Leader (na) | ATGCTTCTCCTGGTGACAAGCCTTCTGCTCTGTGAGTTACCACAC |
| NO: 3205 | | CCAGCATTCCTCCTGATCCCA |
| SEQ ID | Leader (na) | ATGCTGCTGCTGGTGACCAGCCTGCTGCTGTGCGAGCTGCCCCAC |
| NO: 3206 | | CCCGCCTTTCTGCTGATCCCC |
| SEQ ID | CD8 hinge | TTTPAPRPPTPAPTIASQPLSLRPEACRPAAGGAVHTRGLDFACD |
| NO: 1018 | (aa) | |
| SEQ ID | CD8 hinge | ACCACGACGCCAGCGCCGCGACCACCAACACCGGCGCCCACCAT |
| NO: 1019 | (na) | CGCGTCGCAGCCCCTGTCCCTGCGCCCAGAGGCGTGCCGGCCAG |
| | | CGGCGGGGGGCGCAGTGCACACGAGGGGGCTGGACTTCGCCTGT |
| | | GAT |
| SEQ ID | Ig4 hinge | ESKYGPPCPPCPAPEFLGGPSVFLFPPKPKDTLMISRTPEVTCVVVDV |
| NO: 1020 | (aa) | SQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDGS |
| | | FFLYSRLTVDKSRWQEGNVFSCSVMHEALHNHYTQKSLSLSLGKM |
| SEQ ID | Ig4 hinge | GAGAGCAAGTACGGCCCTCCCTGCCCCCCTTGCCCTGCCCCCGA |
| NO: 1021 | (na) | GTTCCTGGGCGGACCCAGCGTGTTCCTGTTCCCCCCCAAGCCCAA |
| | | GGACACCCTGATGATCAGCCGGACCCCCGAGGTGACCTGTGTGG |
| | | TGGTGGACGTGTCCCAGGAGGACCCCGAGGTCCAGTTCAACTGG |
| | | TACGTGGACGGCGTGGAGGTGCACAACGCCAAGACCAAGCCCC |
| | | GGGAGGAGCAGTTCAATAGCACCTACCGGGTGGTGTCCGTGCTG |
| | | ACCGTGCTGCACCAGGACTGGCTGAACGGCAAGGAATACAAGTG |
| | | TAAGGTGTCCAACAAGGGCCTGCCCAGCAGCATCGAGAAAACCA |
| | | TCAGCAAGGCCAAGGGCCAGCCTCGGGAGCCCCAGGTGTACACC |
| | | CTGCCCCCTAGCCAAGAGGAGATGACCAAGAACCAGGTGTCCCT |
| | | GACCTGCCTGGTGAAGGGCTTCTACCCCAGCGACATCGCCGTGG |
| | | AGTGGGAGAGCAACGGCCAGCCCGAGAACAACTACAAGACCAC |
| | | CCCCCCTGTGCTGGACAGCGACGGCAGCTTCTTCCTGTACAGCCG |
| | | GCTGACCGTGGACAAGAGCCGGTGGCAGGAGGGCAACGTCTTTA |
| | | GCTGCTCCGTGATGCACGAGGCCCTGCACAACCACTACACCCAG |
| | | AAGAGCCTGAGCCTGTCCCTGGGCAAGATG |
| SEQ ID | IgD hinge | RWPESPKAQASSVPTAQPQAEGSLAKATTAPATTRNTGRGGEEKK |
| NO: 1022 | (aa) | KEKEKEEQEERETKTPECPSHTQPLGVYLLTPAVQDLWLRDKATFT |
| | | CFVVGSDLKDAHLTWEVAGKVPTGGVEEGLLERHSNGSQSQHSRL |
| | | TLPRSLWNAGTSVTCTLNHPSLPPQRLMALREPAAQAPVKLSLNLL |
| | | ASSDPPEAASWLLCEVSGFSPPNILLMWLEDQREVNTSGFAPARPPP |
| | | QPGSTTFWAWSVLRVPAPPSPQPATYTCVVSHEDSRTLLNASRSLE |
| | | VSYVTDH |
| SEQ ID | IgD hinge | AGGTGGCCCGAAAGTCCCAAGGCCCAGGCATCTAGTGTTCCTAC |
| NO: 1023 | (na) | TGCACAGCCCCAGGCAGAAGGCAGCCTAGCCAAAGCTACTACTG |
| | | CACCTGCCACTACGCGCAATACTGGCCGTGGCGGGGAGGAGAAG |
| | | AAAAAGGAGAAAGAGAAAGAAGAACAGGAAGAGAGGGAGACC |
| | | AAGACCCCTGAATGTCCATCCCATACCCAGCCGCTGGGCGTCTA |
| | | TCTCTTGACTCCCGCAGTACAGGACTTGTGGCTTAGAGATAAGG |
| | | CCACCTTTACATGTTTCGTCGTGGGCTCTGACCTGAAGGATGCCC |
| | | ATTTGACTTGGGAGGTTGCCGGAAAGGTACCCACAGGGGGGGTT |
| | | GAGGAAGGGTTGCTGGAGCGCCATTCCAATGGCTCTCAGAGCCA |
| | | GCACTCAAGACTCACCCTTCCGAGATCCCTGTGGAACGCCGGGA |
| | | CCTCTGTCACATGTACTCTAAATCATCCTAGCCTGCCCCCACAGC |
| | | GTCTGATGGCCCTTAGAGAGCCAGCCGCCCAGGCACCAGTTAAG |
| | | CTTAGCCTGAATCTGCTCGCCAGTAGTGATCCCCCAGAGGCCGC |
| | | CAGCTGGCTCTTATGCGAAGTGTCCGGCTTTAGCCCGCCCAACAT |
| | | CTTGCTCATGTGGCTGGAGGACCAGCGAGAAGTGAACACCAGCG |
| | | GCTTCGCTCCAGCCCGGCCCCCACCCCAGCCGGGTTCTACCACAT |
| | | TCTGGGCCTGGAGTGTCTTAAGGGTCCCAGCACCACCTAGCCCC |
| | | CAGCCAGCCACATACACCTGTGTTGTGTCCCATGAAGATAGCAG |
| | | GACCCTGCTAAATGCTTCTAGGAGTCTGGAGGTTTCCTACGTGAC |
| | | TGACCATT |
| SEQ ID | GS | GGGGSGGGGS |
| NO: 1024 | hinge/linker | |
| | (aa) | |
| SEQ ID | GS | GGTGGCGGAGGTTCTGGAGGTGGAGGTTCC |
| NO: 1025 | hinge/linker | |
| | (na) | |
| SEQ ID | CD8 | IYIWAPLAGTCGVLLLSLVITLYC |
| NO: 1026 | transmembrane | |
| | (TM) (aa) | |
| SEQ ID | CD8 | ATCTACATCTGGGCGCCCTTGGCCGGGACTTGTGGGGTCCTTCTC |
| NO: 1027 | transmembrane | CTGTCACTGGTTATCACCCTTTACTGC |
| | (TM) (na) | |
| SEQ ID | CD8 TM (na) | ATCTACATTTGGGCCCCTCTGGCTGGTACTTGCGGGGTCCTGCTG |
| NO: 1028 | | CTTTCACTCGTGATCACTCTTTACTGT |
| SEQ ID | 4-1BB | KRGRKKLLYIFKQPFMRPVQTTQEEDGCSCRFPEEEEGGCEL |
| NO: 1029 | intracellular | |
| | domain (aa) | |
| SEQ ID | 4-1BB | AAACGGGGCAGAAAGAAACTCCTGTATATATTCAAACAACCATT |
| NO: 1030 | intracellular | TATGAGACCAGTACAAACTACTCAAGAGGAAGATGGCTGTAGCT |
| | domain (na) | GCCGATTTCCAGAAGAAGAAGAAGGAGGATGTGAACTG |
| SEQ ID | 4-1BB | AAGCGCGGTCGGAAGAAGCTGCTGTACATCTTTAAGCAACCCTT |
| NO: 1031 | intracellular | CATGAGGCCTGTGCAGACTACTCAAGAGGAGGACGGCTGTTCAT |
| | domain (na) | GCCGGTTCCCAGAGGAGGAGGAAGGCGGCTGCGAACTG |
| SEQ ID | CD27 (aa) | QRRKYRSNKGESPVEPAEPCRYSCPREEEGSTIPIQEDYRKPEPACSP |
| NO: 1032 | | |
| SEQ ID | CD27 (na) | AGGAGTAAGAGGAGCAGGCTCCTGCACAGTGACTACATGAACAT |
| NO: 1033 | | GACTCCCCGCCGCCCCGGGCCCACCCGCAAGCATTACCAGCCCT |
| | | ATGCCCCACCACGCGACTTCGCAGCCTATCGCTCC |
| SEQ ID | CD3-zeta | RVKFSRSADAPAYKQGQNQLYNELNLGRREEYDVLDKRRGRDPE |
| NO: 1034 | (aa) | MGGKPRRKNPQEGLYNELQKDKMAEAYSEIGMKGERRRGKGHDG |
| | | LYQGLSTATKDTYDALHMQALPPR |
| SEQ ID | CD3-zeta | AGAGTGAAGTTCAGCAGGAGCGCAGACGCCCCCGCGTACAAGC |
| NO: 1035 | (na) | AGGGCCAGAACCAGCTCTATAACGAGCTCAATCTAGGACGAAGA |
| | | GAGGAGTACGATGTTTTGGACAAGAGACGTGGCCGGGACCCTGA |
| | | GATGGGGGGAAAGCCGAGAAGGAAGAACCCTCAGGAAGGCCTG |
| | | TACAATGAACTGCAGAAAGATAAGATGGCGGAGGCCTACAGTG |
| | | AGATTGGGATGAAAGGCGAGCGCCGGAGGGGCAAGGGGCACGA |
| | | TGGCCTTTACCAGGGTCTCAGTACAGCCACCAAGGACACCTACG |
| | | ACGCCCTTCACATGCAGGCCCTGCCCCCTCGC |
| SEQ ID | CD3-zeta | CGCGTGAAATTCAGCCGCAGCGCAGATGCTCCAGCCTACAAGCA |
| NO: 1036 | (na) | GGGGCAGAACCAGCTCTACAACGAACTCAATCTTGGTCGGAGAG |
| | | AGGAGTACGACGTGCTGGACAAGCGGAGAGGACGGGACCCAGA |
| | | AATGGGCGGGAAGCCGCGCAGAAAGAATCCCCAAGAGGGCCTG |
| | | TACAACGAGCTCCAAAAGGATAAGATGGCAGAAGCCTATAGCG |
| | | AGATTGGTATGAAAGGGGAACGCAGAAGAGGCAAAGGCCACGA |
| | | CGGACTGTACCAGGGACTCAGCACCGCCACCAAGGACACCTATG |
| | | ACGCTCTTCACATGCAGGCCCTGCCGCCTCGG |
| SEQ ID | CD3-zeta | RVKFSRSADAPAYQQGQNQLYNELNLGRREEYDVLDKRRGRDPE |
| NO: 1037 | (aa) | MGGKPRRKNPQEGLYNELQKDKMAEAYSEIGMKGERRRGKGHDG |
| | | LYQGLSTATKDTYDALHMQALPPR |
| SEQ ID | CD3-zeta | AGAGTGAAGTTCAGCAGGAGCGCAGACGCCCCCGCGTACCAGC |
| NO: 1038 | (na) | AGGGCCAGAACCAGCTCTATAACGAGCTCAATCTAGGACGAAGA |
| | | GAGGAGTACGATGTTTTGGACAAGAGACGTGGCCGGGACCCTGA |
| | | GATGGGGGGAAAGCCGAGAAGGAAGAACCCTCAGGAAGGCCTG |
| | | TACAATGAACTGCAGAAAGATAAGATGGCGGAGGCCTACAGTG |
| | | AGATTGGGATGAAAGGCGAGCGCCGGAGGGGCAAGGGGCACGA |
| | | TGGCCTTTACCAGGGTCTCAGTACAGCCACCAAGGACACCTACG |
| | | ACGCCCTTCACATGCAGGCCCTGCCCCCTCGC |
| SEQ ID | linker | GGGGS |
| NO: 1039 | | |
| SEQ ID | linker | GGTGGCGGAGGTTCTGGAGGTGGAGGTTCC |
| NO: 1040 | | |
| SEQ ID | PD-1 | Pgwfldspdrpwnpptfspallvvtegdnatftcsfsntsesfvlnwyrmspsnqtdklaafpedrsq |
| NO: 1041 | extracellular | pgqdcrfrvtqlpngrdfhmsvvrarrndsgtylegaislapkaqikeslraelrvterraevptahp |
| | domain (aa) | spsprpagqfqtlv |
| SEQ ID | PD-1 | Cccggatggtttctggactctccggatcgcccgtggaatcccccaaccttctcaccggcactcttggt |
| NO: 1042 | extracellular | tgtgactgagggcgataatgcgaccttcacgtgctcgttctccaacacctccgaatcattcgtgctga |
| | domain (na) | actggtaccgcatgagcccgtcaaaccagaccgacaagctcgccgcgtttccggaagatcggtcgcaa |
| | | ccgggacaggattgtcggttccgcgtgactcaactgccgaatggcagagacttccacatgagcgtggt |
| | | ccgcgctaggcgaaacgactccgggacctacctgtgcggagccatctcgctggcgcctaaggcccaaa |
| | | tcaaagagagcttgagggccgaactgagagtgaccgagcgcagagctgaggtgccaactgcacatcca |
| | | tccccatcgcctcggcctgcggggcagtttcagaccctggtc |
| SEQ ID | PD-1 CAR | Malpvtalllplalllhaarppgwfldspdrpwnpptfspallvvtegdnatftcsfsntsesfvlnw |
| NO: 1043 | (aa) with | yrmspsnqtdklaafpedrsqpgqdcrfrvtqlpngrdfhmsvvrarrndsgtylcgaislapkaqik |
| | signal | eslraelrvterraevptahpspsprpagqfqtlvtttpaprpptpaptiasqplslrpeacrpaagg |
| | | avhtrgldfacdiyiwaplagtcgvlllslvitlyckrgrkkllyifkqpfmrpvqttqeedgcscrf |
| | | peeeeggcelrvkfsrsadapaykqgqnqlynelnlgrreeydvldkrrgrdpemggkprrknpqegl |
| | | ynelqkdkmaeayseigmkgerrrgkghdglyqglstatkdtydalhmqalppr |
| SEQ ID | PD-1 CAR | Atggccctccctgtcactgccctgcttctccccctcgcactcctgctccacgccgctagaccacccgg |
| NO: 1044 | (na) | atggtttctggactctccggatcgcccgtggaatcccccaaccttctcaccggcactcttggttgtga |
| | | ctgagggcgataatgcgaccttcacgtgctcgttctccaacacctccgaatcattcgtgctgaactgg |
| | | taccgcatgagcccgtcaaaccagaccgacaagctcgccgcgtttccggaagatcggtcgcaaccggg |
| | | acaggattgtcggttccgcgtgactcaactgccgaatggcagagacttccacatgagcgtggtccgcg |
| | | ctaggcgaaacgactccgggacctacctgtgcggagccatctcgctggcgcctaaggcccaaatcaaa |
| | | gagagcttgagggccgaactgagagtgaccgagcgcagagctgaggtgccaactgcacatccatcccc |
| | | atcgcctcggcctgcggggcagtttcagaccctggtcacgaccactccggcgccgcgcccaccgactc |
| | | cggccccaactatcgcgagccagcccctgtcgctgaggccggaagcatgccgccctgccgccggaggt |
| | | gctgtgcatacccggggattggacttcgcatgcgacatctacatttgggctcctctcgccggaacttg |
| | | tggcgtgctccttctgtccctggtcatcaccctgtactgcaagcggggtcggaaaaagcttctgtaca |
| | | ttttcaagcagcccttcatgaggcccgtgcaaaccacccaggaggaggacggttgctcctgccggttc |
| | | cccgaagaggaagaaggaggttgcgagctgcgcgtgaagttctcccggagcgccgacgcccccgccta |
| | | taagcagggccagaaccagctgtacaacgaactgaacctgggacggcgggaagagtacgatgtgctgg |
| | | acaagcggcgcggccgggaccccgaaatgggcgggaagcctagaagaaagaaccctcaggaaggcctg |
| | | tataacgagctgcagaaggacaagatggccgaggcctactccgaaattgggatgaagggagagcggcg |
| | | gaggggaaaggggcacgacggcctgtaccaaggactgtccaccgccaccaaggacacatacgatgccc |
| | | tgcacatgcaggcccttccccctcgc |
| SEQ ID | linker | (Gly-Gly-Gly-Ser)n, where n = 1-10 |
| NO: 3200 | | |
| SEQ ID | linker | (Gly4 Ser)4 |
| NO: 1010 | | |
| SEQ ID | linker | (Gly4 Ser)3 |
| NO: 1011 | | |
| SEQ ID | linker | (Gly3Ser) |
| NO: 1012 | | |
| SEQ ID | linker | ASGGGGSGGRASGGGGS |
| NO: 1045 | | |
| SEQ ID | polyA | [a]₅₀₋₅₀₀₀ |
| NO: 1013 | | |
| SEQ ID | PD1 CAR | Pgwfldspdrpwnpptfspallvvtegdnatftcsfsntsesfvlnwyrmspsnqtdklaafpedrsq |
| NO: 1046 | (aa) | pgqdcrfrvtqlpngrdfhmsvvrarrndsgtylcgaislapkaqikeslraelrvterraevptahp |
| | | spsprpagqfqtlvtttpaprpptpaptiasqplslrpeacrpaaggavhtrgldfacdiyiwaplag |
| | | tcgvlllslvitlyckrgrkkllyifkqpfmrpvqttqeedgcscrfpeeeeggcelrvkfsrsadap |
| | | aykqgqnqlynelnlgrreeydvldkrrgrdpemggkprrknpqeglynelqkdkmaeayseigmkge |
| | | rrrgkghdglyqglstatkdtydalhmqalppr |
| SEQ ID | ICOS | TKKKYSSSVHDPNGEYMFMRAVNTAKKSRLTDVTL |
| NO: 1047 | intracellular | |
| | domain (aa) | |
| SEQ ID | ICOS | ACAAAAAAGAAGTATTCATCCAGTGTGCACGACCCTAACGGTGA |
| NO: 1048 | intracellular | ATACATGTTCATGAGAGCAGTGAACACAGCCAAAAAATCCAGAC |
| | domain (na) | TCACAGATGTGACCCTA |
| SEQ ID | ICOS TM | TTTPAPRPPTPAPTIASQPLSLRPEACRPAAGGAVHTRGLDFACDFW |
| NO: 1049 | domain (aa) | LPIGCAAFVVVCILGCILICWL |
| SEQ ID | ICOS TM | ACCACGACGCCAGCGCCGCGACCACCAACACCGGCGCCCACCAT |
| NO: 1050 | domain (na) | CGCGTCGCAGCCCCTGTCCCTGCGCCCAGAGGCGTGCCGGCCAG |
| | | CGGCGGGGGGCGCAGTGCACACGAGGGGGCTGGACTTCGCCTGT |
| | | GATTICTGGTTACCCATAGGATGTGCAGCCTTTGTTGTAGTCTGC |
| | | ATTTTGGGATGCATACTTATTTGTTGGCTT |
| SEQ ID | CD28 | RSKRSRLLHSDYMNMTPRRPGPTRKHYQPYAPPRDFAAYRS |
| NO: 1051 | intracellular | |
| | domain (aa) | |
| SEQ ID | CD28 | AGGAGTAAGAGGAGCAGGCTCCTGCACAGTGACTACATGAACAT |
| NO: 1052 | intracellular | GACTCCCCGCCGCCCCGGGCCCACCCGCAAGCATTACCAGCCCT |
| | domain (na) | ATGCCCCACCACGCGACTTCGCAGCCTATCGCTCC |

### CAR Antigen Binding Domain

In some embodiments, the portion of the CAR molecule comprising the antigen-binding domain comprises an antigen-binding domain that targets a tumor antigen, e.g., a tumor antigen described herein. In some embodiments, the antigen binding domain binds to: CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; ν-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECI2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

The antigen binding domain can be any domain that binds to an antigen, including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen-binding domain to be derived from the same species in which the CAR molecule will ultimately be used in. For example, for use in humans, it may be beneficial for the antigen binding domain of the CAR molecule to comprise human or humanized residues for the antigen-binding domain of an antibody or antibody fragment.

In one embodiment, the CD19 CAR is a CD19 CAR described in U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); or 16th Annu Meet Am Soc Gen Cell Ther (ASGCT) (May 15-18, Salt Lake City) 2013, Abst 10 (each of which is herein incorporated by reference in their entirety). In one embodiment, an antigen binding domain against CD19 is an antigen binding portion, e.g., CDRs, of a CAR molecule, antibody or antigen-binding fragment thereof described in, e.g., PCT publication WO2012/079000 (incorporated herein by reference in its entirety). In one embodiment, an antigen binding domain against CD19 is an antigen binding portion, e.g., CDRs, of a CAR molecule, antibody or antigen-binding fragment thereof described in, e.g., PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190 (each of which is herein incorporated by reference in their entirety).

In one embodiment, the antigen binding domain against mesothelin is or may be derived from an antigen binding domain, e.g., CDRs, scFv, or VH and VL, of an antibody, antigen-binding fragment or CAR molecule described in, e.g., PCT publication WO2015/090230 (In one embodiment the CAR molecule is a CAR molecule described in WO2015/090230, the contents of which are incorporated herein in their entirety). In some embodiments, the antigen binding domain against mesothelin is or is derived from an antigen binding portion, e.g., CDRs, scFv, or VH and VL, of an antibody, antigen-binding fragment, or CAR molecule described in, e.g., PCT publication WO1997/025068, WO1999/028471, WO2005/014652, WO2006/099141, WO2009/045957, WO2009/068204, WO2013/142034, WO2013/040557, or WO2013/063419 (each of which is herein incorporated by reference in their entirety).

In one embodiment, an antigen-binding domain against CD123 is or is derived from an antigen-binding portion, e.g., CDRs, scFv or VH and VL, of an antibody, antigen-binding fragment or CAR molecule described in, e.g., PCT publication WO2014/130635 (incorporated herein by reference in its entirety). In one embodiment, an antigen binding domain against CD123 is or is derived from an antigen binding portion, e.g., CDRs, scFv or VH and VL, of an antibody, antigen-binding fragment or CAR molecule described in, e.g., PCT publication WO2016/028896 (incorporated herein by reference in its entirety); in some embodiments, the CAR molecule is a CAR molecule described in WO2016/028896. In one embodiment, an antigen binding domain against CD123 is or is derived from an antigen binding portion, e.g., CDRs, scFv, or VL and VH, of an antibody, antigen-binding fragment, or CAR molecule described in, e.g., PCT publication WO1997/024373, WO2008/127735 (e.g., a CD123 binding domain of 26292, 32701, 37716 or 32703), WO2014/138805 (e.g., a CD123 binding domain of CSL362), WO2014/138819, WO2013/173820, WO2014/144622, WO2001/66139, WO2010/126066 (e.g., the CD123 binding domain of any of Old4, Old5, O1d17, O1d19, New102, or Old6), WO2014/144622, or US2009/0252742 (each of which is incorporated herein by reference in its entirety).

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen-binding domain against CS-1 is an antigen-binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen-binding domain against CLL-1 is an antigen-binding portion, e.g., CDRs or VH and VL, of an antibody, antigen-binding fragment or CAR molecule described in, e.g., PCT publication WO2016/014535, the contents of which are incorporated herein in their entirety. In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD).

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014). Exemplary CAR molecules that target CD33 are described herein, and are provided in WO2016/014576, e.g., in Table 2 of WO2016/014576 (incorporated by reference in its entirety).

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen-binding portion, e.g., CDRs, of an antibody described in, e.g., WO2012163805, WO200112812, and WO2003062401. In some embodiments, additional exemplary BCMA CAR constructs are generated using an antigen binding domain, e.g., CDRs, scFv, or VH and VL sequences from PCT Publication WO2012/0163805 (the contents of which are hereby incorporated by reference in its entirety). In some embodiments, additional exemplary BCMA CAR constructs are generated using an antigen binding domain, e.g., CDRs, scFv, or VH and VL sequences from PCT Publication WO2016/014565 (the contents of which are hereby incorporated by reference in its entirety). In some embodiments, additional exemplary BCMA CAR constructs are generated using an antigen binding domain, e.g., CDRs, scFv, or VH and VL sequences from PCT Publication WO2014/122144 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2016/014789 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2014/089335 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2014/140248 (the contents of which are hereby incorporated by reference in its entirety).

In one embodiment, an antigen binding domain against Tn antigen is an antigen binding portion, e.g., CDRs, of an antibody described in, e.g., US 2014/0178365, U.S. Pat. No. 8,440,798, Brooks et al., PNAS 107(22):10056-10061 (2010), and Stone et al., Oncolmmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen-binding domain against FLT3 is an antigen-binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen-binding portion, e.g., CDRs, of an antibody described in, e.g., Hombach et al., Gastroenterology 113(4):1163-1170 (1997); and Abcam ab691.

In one embodiment, an antigen binding domain against FAP is an antigen-binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen-binding portion, e.g., CDRs, of an antibody described in, e.g., Casucci et al., Blood 122(20):3461-3472 (2013).

In one embodiment, an antigen binding domain against CEA is an antigen-binding portion, e.g., CDRs, of an antibody described in, e.g., Chmielewski et al., Gastoenterology 143(4):1095-1107 (2012).

In one embodiment, an antigen-binding domain against EPCAM is an antigen-binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen-binding domain against PRSS21 is an antigen-binding portion, e.g., CDRS, of an antibody described in U.S. Pat. No. 8,080,650.

In one embodiment, an antigen-binding domain against B7H3 is an antigen-binding portion, e.g., CDRS, of an antibody MGA271 (Macrogenics).

In one embodiment, an antigen-binding domain against KIT is an antigen-binding portion, e.g., CDRS, of an antibody described in, e.g., U.S. Pat. No. 7,915,391, US20120288506, and several commercial catalog antibodies.

In one embodiment, an antigen-binding domain against IL-13Ra2 is an antigen-binding portion, e.g., CDRs, of an antibody described in, e.g., WO2008/146911, WO2004087758, several commercial catalog antibodies, and WO2004087758.

In one embodiment, an antigen-binding domain against CD30 is an antigen-binding portion, e.g., CDRS, of an antibody described in, e.g., U.S. Pat. No. 7,090,843 B1, and EP0805871.

In one embodiment, an antigen-binding domain against GD3 is an antigen-binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRS, of an antibody described in, e.g., Hong et al., J Immunother 37(2):93-104 (2014).

In one embodiment, an antigen-binding domain against IL-11Ra is an antigen-binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131(2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen-binding domain against PDGFR-beta is an antigen-binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen-binding domain against CD20 is an antigen-binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen-binding portion, e.g., CDRs, of the antibody trastuzumab, or pertuzumab.

In one embodiment, an antigen-binding domain against MUC1 is an antigen-binding portion, e.g., CDRs, of the antibody SAR566658.

In one embodiment, the antigen-binding domain against EGFR is antigen-binding portion, e.g., CDRs, of the antibody cetuximab, panitumumab, zalutumumab, nimotuzumab, or matuzumab. In one embodiment, the antigen binding domain against EGFRvIII is or may be derived from an antigen binding domain, e.g., CDRs, scFv, or VH and VL, of an antibody, antigen-binding fragment or CAR molecule described in, e.g., PCT publication WO2014/130657 (In one embodiment the CAR molecule is a CAR molecule described in WO2014/130657, the contents of which are incorporated herein in their entirety).

In one embodiment, an antigen binding domain against NCAM is an antigen binding portion, e.g., CDRs, of the antibody clone 2-2B: MAB5324 (EMD Millipore)

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen-binding domain against CAIX is an antigen-binding portion, e.g., CDRs, of the antibody clone 303123 (R&D Systems).

In one embodiment, an antigen-binding domain against LMP2 is an antigen-binding portion, e.g., CDRs, of an antibody described in, e.g., U.S. Pat. No. 7,410,640, or US20050129701.

In one embodiment, an antigen-binding domain against gp100 is an antigen-binding portion, e.g., CDRs, of the antibody HMB45, NKIbetaB, or an antibody described in WO2013165940, or US20130295007.

In one embodiment, an antigen-binding domain against tyrosinase is an antigen-binding portion, e.g., CDRs, of an antibody described in, e.g., U.S. Pat. No. 5,843,674; or U.S. Ser. No. 19/950,504,048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen-binding domain against GD3 is an antigen-binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen-binding domain against fucosyl GM1 is an antigen-binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott AM et al, Cancer Res 60: 3254-61 (2000), also as described in Neeson et al, J Immunol May 2013 190 (Meeting Abstract Supplement) 177.10.

In one embodiment, an antigen-binding domain against GM3 is an antigen-binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen-binding domain against o-acetyl-GD2 is an antigen-binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMABO27 (Ganymed Pharmaceuticals), see e.g., clinicaltrial.gov/show/NCT02054351.

In one embodiment, an antigen-binding domain against TSHR is an antigen-binding portion, e.g., CDRs, of an antibody described in, e.g., U.S. Pat. Nos. 8,603,466; 8,501,415; or U.S. Pat. No. 8,309,693.

In one embodiment, an antigen-binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen-binding domain against ALK is an antigen binding portion, e.g., CDRs, of an antibody described in, e.g., Mino-Kenudson et al., Clin Cancer Res 16(5):1561-1571 (2010).

In one embodiment, an antigen-binding domain against polysialic acid is an antigen binding portion, e.g., CDRs, of an antibody described in, e.g., Nagae et al., J Biol Chem 288(47):33784-33796 (2013).

In one embodiment, an antigen-binding domain against PLAC1 is an antigen binding portion, e.g., CDRs, of an antibody described in, e.g., Ghods et al., Biotechnol Appl Biochem 2013 doi: 10.1002/bab.1177.

In one embodiment, an antigen-binding domain against GloboH is an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBrl: Bremer E-G et al. J Biol Chem 259:14773-14777 (1984).

In one embodiment, an antigen-binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen-binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen-binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen-binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen-binding domain against Tie 2 is an antigen-binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen-binding domain against Fos-related antigen 1 is an antigen-binding portion, e.g., CDRs, of the antibody 12F9 (Novus Biologicals).

In one embodiment, an antigen-binding domain against MelanA/MART1 is an antigen-binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYPIB1 is an antigen binding portion, e.g., CDRs, of an antibody described in, e.g., Maecker et al, Blood 102 (9): 3287-3294 (2003).

In one embodiment, an antigen-binding domain against RAGE-1 is an antigen-binding portion, e.g., CDRs, of the antibody MAB5328 (EMD Millipore).

In one embodiment, an antigen-binding domain against human telomerase reverse transcriptase is an antigen-binding portion, e.g., CDRs, of the antibody cat no: LS-B95-100 (Lifespan Biosciences)

In one embodiment, an antigen-binding domain against intestinal carboxyl esterase is an antigen-binding portion, e.g., CDRs, of the antibody 4F12: cat no: LS-B6190-50 (Lifespan Biosciences).

In one embodiment, an antigen-binding domain against mut hsp70-2 is an antigen-binding portion, e.g., CDRs, of the antibody Lifespan Biosciences: monoclonal: cat no: LS-C133261-100 (Lifespan Biosciences).

In one embodiment, an antigen-binding domain against CD79a is an antigen-binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Doman et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, CA Dec. 6-9 2014.

In one embodiment, an antigen-binding domain against CD72 is an antigen-binding portion, e.g., CDRs, of the antibody J3-109 described in Myers, and Uckun, “An anti-CD72 μmmunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen-binding domain against LAIR1 is an antigen-binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1×CD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal [494025] available from R&D Systems.

In one embodiment, an antigen-binding domain against LY75 is an antigen-binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen-binding domain against GPC3 is an antigen-binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen-binding domain against FCRL5 is an antigen-binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32.

In one embodiment, an antigen-binding domain against IGLL1 is an antigen-binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen-binding domain comprises a heavy chain variable region and/or a variable light chain region of an antibody listed above.

In another aspect, the antigen-binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen-binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., from the amino acid at the corresponding murine sequence.

In some aspects, the portion of a CAR composition of the disclosure that comprises an antibody fragment is humanized with retention of high affinity for the target antigen and other favorable biological properties. According to one aspect of the disclosure, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind human a cancer associated antigen as described herein. In some embodiments, a humanized antibody or antibody fragment may have improved affinity and/or specificity of binding to human a cancer associated antigen as described herein.

In one aspect, the antigen-binding domain of the disclosure is characterized by particular functional features or properties of an antibody or antibody fragment. For example, in one aspect, the portion of a CAR composition of the disclosure that comprises an antigen-binding domain specifically binds a tumor antigen as described herein.

In one aspect, the anti-cancer associated antigen as described herein binding domain is a fragment, e.g., a single chain variable fragment (scFv). In one aspect, the anti-cancer associated antigen as described herein binding domain is a Fv, a Fab, a (Fab′)₂, or a bi-functional (e.g. bi-specific) hybrid antibody (e.g., Lanzavecchia et al., Eur. J. Immunol. 17, 105 (1987)). In one aspect, the antibodies and fragments thereof of the disclosure binds a cancer associated antigen as described herein protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 3000). In one embodiment, the linker can be (Gly₄Ser)₄ (SEQ ID NO: 3001) or (Gly₄Ser)₃(SEQ ID NO: 3002). Variation in the linker length may retain or enhance activity, giving rise to superior efficacy in activity studies.

In another aspect, the antigen-binding domain is a T cell receptor (“TCR”), or a fragment thereof, for example, a single chain TCR (scTCR). Methods to make such TCRs are known in the art. See, e.g., Willemsen RA et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Va and VD genes from a T cell clone linked by a linker (e.g., a flexible peptide). This approach is very useful to cancer associated target that itself is intracellar, however, a fragment of such antigen (peptide) is presented on the surface of the cancer cells by MHC.

In one embodiment, an antigen-binding domain against EGFRvIII is an antigen-binding portion, e.g., CDRs, of a CAR molecule, antibody or antigen-binding fragment thereof described in, e.g., PCT publication WO2014/130657 or US2014/0322275A1. In one embodiment, the CAR molecule comprises an EGFRvIII CAR, or an antigen binding domain according to Table 2 or SEQ ID NO: 11 of WO 2014/130657, incorporated herein by reference, or a sequence substantially identical thereto (e.g., at least 85%, 90%, 95% or more identical thereto). The amino acid and nucleotide sequences encoding the EGFRvIII CAR molecules and antigen binding domains (e.g., including one, two, three VH CDRs; and one, two, three VL CDRs according to Kabat or Chothia), are specified in WO 2014/130657.

In one embodiment, an antigen-binding domain against mesothelin is an antigen-binding portion, e.g., CDRs, of an antibody, antigen-binding fragment or CAR molecule described in, e.g., PCT publication WO2015/090230. In one embodiment, an antigen-binding domain against mesothelin is an antigen-binding portion, e.g., CDRs, of an antibody, antigen-binding fragment, or CAR molecule described in, e.g., PCT publication WO1997/025068, WO1999/028471, WO2005/014652, WO2006/099141, WO2009/045957, WO2009/068204, WO2013/142034, WO2013/040557, or WO2013/063419.

In an embodiment, the CAR molecule comprises a mesothelin CAR described herein, e.g., a mesothelin CAR described in WO 2015/090230, incorporated herein by reference. In some embodiments, the mesothelin CAR comprises an amino acid, or has a nucleotide sequence shown in WO 2015/090230 incorporated herein by reference, or a sequence substantially identical to any of the aforesaid sequences (e.g., at least 85%, 90%, 95% or more identical to any of the aforesaid mesothelin CAR sequences). In one embodiment, the CAR molecule comprises a mesothelin CAR, or an antigen binding domain according to Tables 2-3 of WO 2015/090230, incorporated herein by reference, or a sequence substantially identical thereto (e.g., at least 85%, 90%, 95% or more identical thereto). The amino acid and nucleotide sequences encoding the mesothelin CAR molecules and antigen binding domains (e.g., including one, two, three VH CDRs; and one, two, three VL CDRs according to Kabat or Chothia), are specified in WO 2015/090230.

### CAR Transmembrane Domain

With respect to the transmembrane domain, in various embodiments, a CAR molecule can be designed to comprise a transmembrane domain that is attached to the extracellular domain of the CAR molecule. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR molecule. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR molecule on the cell surface of a CAR-expressing cell. In some embodiments, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In some embodiments, the transmembrane domain is capable of signaling to the intracellular domain(s) whenever the CAR molecule has bound to a target. A transmembrane domain of particular use in this disclosure may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIR2DS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C.

In some instances, the transmembrane domain can be attached to the extracellular region of the CAR molecule, e.g., the antigen-binding domain of the CAR molecule, via a hinge, e.g., a hinge from a human protein. For example, in some embodiments, the hinge can be a human Ig (immunoglobulin) hinge, e.g., an IgG4 hinge, or a CD8a hinge. In some embodiments, the hinge or spacer comprises (e.g., consists of) the amino acid sequence of SEQ ID NO: 1018. In some embodiments, the transmembrane domain comprises (e.g., consists of) a transmembrane domain of SEQ ID NO: 1026.

In some embodiments, the hinge or spacer comprises an IgG4 hinge. For example, in some embodiments, the hinge or spacer comprises a hinge of the amino acid sequence of SEQ ID NO: 1020. In some embodiments, the hinge or spacer comprises a hinge encoded by a nucleotide sequence of SEQ ID NO: 1021.

In some embodiments, the hinge or spacer comprises an IgD hinge. For example, in some embodiments, the hinge or spacer comprises a hinge of the amino acid sequence of SEQ ID NO: 1022. In some embodiments, the hinge or spacer comprises a hinge encoded by a nucleotide sequence of SEQ ID NO: 1023.

In some embodiments, the transmembrane domain may be recombinant, in which case it will comprise predominantly hydrophobic residues such as leucine and valine. In some embodiments, a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR molecule. A glycine-serine doublet provides a particularly suitable linker. For example, in some embodiments, the linker comprises the amino acid sequence of SEQ ID NO: 1024. In some embodiments, the linker is encoded by a nucleotide sequence of SEQ ID NO: 1025.

In some embodiments, the hinge or spacer comprises a KIR2DS2 hinge.

### Cytoplasmic Domain

The cytoplasmic domain or region of the CAR molecule includes an intracellular signaling domain. An intracellular signaling domain is generally responsible for activation of at least one of the normal effector functions of the immune cell in which the CAR molecule has been introduced.

Examples of intracellular signaling domains for use in a CAR molecule described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the disclosure include those of TCR zeta, FcR gamma, FcR beta, CD3 gamma, CD3 delta, CD3 epsilon, CD5, CD22, CD79a, CD79b, CD278 (also known as “ICOS”), FεRI, DAP10, DAP12, and CD66d. In some embodiments, a CAR molecule of the disclosure comprises an intracellular signaling domain, e.g., a primary signaling domain of CD3-zeta, e.g., a CD3-zeta sequence described herein.

In some embodiments, a primary signaling domain comprises a modified ITAM domain, e.g., a mutated ITAM domain which has altered (e.g., increased or decreased) activity as compared to the native ITAM domain. In some embodiments, a primary signaling domain comprises a modified ITAM-containing primary intracellular signaling domain, e.g., an optimized and/or truncated ITAM-containing primary intracellular signaling domain. In some embodiments, a primary signaling domain comprises one, two, three, four or more ITAM motifs.

### Costimulatory Signaling Domain

The intracellular signalling domain of the CAR molecule can comprise the CD3-zeta signaling domain by itself or it can be combined with any other desired intracellular signaling domain(s) useful in the context of a CAR molecule of the disclosure. For example, the intracellular signaling domain of the CAR can comprise a CD3 zeta chain portion and a costimulatory signaling domain. The costimulatory signaling domain refers to a portion of the CAR molecule comprising the intracellular domain of a costimulatory molecule. In some embodiments, the intracellular domain is designed to comprise the signaling domain of CD3-zeta and the signaling domain of CD28. In some embodiments, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp30, NKp44, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, NKG2D, NKG2C and PAG/Cbp.

The intracellular signaling sequences within the cytoplasmic portion of the CAR molecule may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In some embodiments, a glycine-senne doublet can be used as a suitable linker. In some embodiments, a single amino acid, e.g., an alanine, a glycine, can be used as a suitable linker.

In some embodiments, the intracellular signaling domain is designed to comprise two or more, e.g., 2, 3, 4, 5, or more, costimulatory signaling domains. In some embodiments, the two or more, e.g., 2, 3, 4, 5, or more, costimulatory signaling domains, are separated by a linker molecule, e.g., a linker molecule described herein. In some embodiments, the intracellular signaling domain comprises two costimulatory signaling domains. In some embodiments, the linker molecule is a glycine residue. In some embodiments, the linker is an alanine residue.

In some embodiments, the intracellular signaling domain is designed to comprise the signaling domain of CD3-zeta and the signaling domain of CD28. In some embodiments, the intracellular signaling domain is designed to comprise the signaling domain of CD3-zeta and the signaling domain of 4-1BB. In some embodiments, the signaling domain of 4-1BB is a signaling domain of SEQ ID NO: 1029. In some embodiments, the signaling domain of CD3-zeta is a signaling domain of SEQ ID NO: 1034.

In some embodiments, the intracellular signaling domain is designed to comprise the signaling domain of CD3-zeta and the signaling domain of CD27. In some embodiments, the signaling domain of CD27 comprises an amino acid sequence of SEQ ID NO: 1032. In some embodiments, the signalling domain of CD27 is encoded by a nucleic acid sequence of SEQ ID NO: 1033.

In some embodiments, the CAR cell described herein can further comprise a second CAR molecule, e.g., a second CAR molecule that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In some embodiments, the second CAR molecule includes an antigen binding domain to a target expressed the same cancer cell type as the cancer associated antigen. In some embodiments, the CAR-expressing cell comprises a first CAR molecule that targets a first antigen and includes an intracellular signaling domain having a costimulatory signaling domain but not a primary signaling domain, and a second CAR molecule that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, ICOS, CD27 or OX-40, onto the first CAR molecule, and the primary signaling domain, e.g., CD3 zeta, on the second CAR molecule can limit the CAR activity to cells where both targets are expressed. In some embodiments, the CAR expressing cell comprises a first cancer associated antigen CAR that includes an antigen binding domain that binds a target antigen described herein, a transmembrane domain and a costimulatory domain and a second CAR molecule that targets a different target antigen (e.g., an antigen expressed on that same cancer cell type as the first target antigen) and includes an antigen binding domain, a transmembrane domain and a primary signaling domain. In some embodiments, the CAR expressing cell comprises a first CAR molecule that includes an antigen binding domain that binds a target antigen described herein, a transmembrane domain and a primary signaling domain and a second CAR molecule that targets an antigen other than the first target antigen (e.g., an antigen expressed on the same cancer cell type as the first target antigen) and includes an antigen binding domain to the antigen, a transmembrane domain and a costimulatory signaling domain.

In some embodiments, the disclosure features a population of CAR cell, e.g., CART cells. In some embodiments, the population of CAR cells comprises a mixture of cells expressing different CAR molecules. For example, in some embodiments, the population of CART cells can include a first cell expressing a CAR molecule having an antigen binding domain to a cancer associated antigen described herein, and a second cell expressing a CAR molecule having a different antigen binding domain, e.g., an antigen binding domain to a different a cancer associated antigen described herein, e.g., an antigen binding domain to a cancer associated antigen described herein that differs from the cancer associate antigen bound by the antigen binding domain of the CAR molecule expressed by the first cell. As another example, the population of CAR cells can include a first cell expressing a CAR molecule that includes an antigen-binding domain to a cancer associated antigen described herein, and a second cell expressing a CAR molecule that includes an antigen-binding domain to a target other than a cancer associate antigen as described herein. In some embodiments, the population of CAR cells includes, e.g., a first cell expressing a CAR molecule that includes a primary intracellular signaling domain, and a second cell expressing a CAR molecule that includes a secondary signaling domain.

In some embodiments, the disclosure features a population of cells wherein at least one cell in the population expresses a CAR molecule having an antigen-binding domain to a cancer associated antigen described herein, and a second cell expressing another agent, e.g., an agent which enhances the activity of a CAR-expressing cell. For example, in some embodiments, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSFi4 or CD270), KIR, A2aR, MHC class I, MHC class II, GAL9, adenosine, and TGF (e.g., TGFbeta). In some embodiments, the agent which inhibits an inhibitory molecule comprises a first polypeptide, e.g., an inhibitory molecule, associated with a second polypeptide that provides a positive signal to the cell, e.g., an intracellular signaling domain described herein. In some embodiments, the agent comprises a first polypeptide, e.g., of an inhibitory molecule such as PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4 and TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In some embodiments, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

### CD19 CAR and CD19-Binding Sequences

In some embodiments, the CAR or CAR-expressing cell described herein is a CD19 CAR-expressing cell (e.g., a cell expressing a CAR molecule that binds to human CD19).

In some embodiments, the antigen-binding domain of the CD19 CAR has the same or a similar binding specificity as the FMC63 scFv fragment described in Nicholson et al. Mol. Immun. 34 (16-17): 1157-1165 (1997). In some embodiments, the antigen-binding domain of the CD19 CAR includes the scFv fragment described in Nicholson et al. Mol. Immun. 34 (16-17): 1157-1165 (1997).

In some embodiments, the CD19 CAR includes an antigen-binding domain (e.g., a humanized antigen binding domain) according to Table 3 of WO2014/153270, incorporated herein by reference. WO2014/153270 also describes methods of assaying the binding and efficacy of various CAR constructs.

In some embodiments, the parental murine scFv sequence is the CAR19 construct provided in PCT publication WO2012/079000 (incorporated herein by reference). In some embodiments, the anti-CD19 binding domain is a scFv described in WO2012/079000.

In some embodiments, the CAR molecule comprises the fusion polypeptide sequence provided as SEQ ID NO: 12 in PCT publication WO2012/079000, which provides an scFv fragment of murine origin that specifically binds to human CD19.

In some embodiments, the CD19 CAR comprises an amino acid sequence provided as SEQ ID NO: 12 in PCT publication WO2012/079000. In some embodiments, the amino acid sequence is (MALPVTALLLPLALLLHAARP)digmtqttsslsaslgdrvtiscrasqdiskylnwyqqkpdgtvklliyhtsrlhsgvpsrfs gsgsgtdysltisnlegediatyfcqqgntlpytfgggtkleitggggsggggsggggsevklqesgpglvapsqslsvtctvsgvslpdygvsw irqpprkglewlgviwgsettyynsalksrltiikdnsksqvflkmnslqtddtaiyycakhyyyggsyamdywgqgtsvtvsstttpaprppt paptiasqplslrpeacrpaaggavhtrgldfacdiyiwaplagtcgvlllslvitlyckrgrkkllyifkqpfmrpvqttqeedgcscrfpeeeeg gcelrvkfsrsadapaykqgqnqlynelnlgrreeydvldkrrgrdpemggkprrknpqeglynelqkdkmaeayseigmkgerrrgkgh dglyqglstatkdtydalhmqalppr (SEQ ID NO: 1053), or a sequence substantially homologous thereto. The optional sequence of the signal peptide is shown in capital letters and parenthesis.

In some embodiments, the amino acid sequence is: digmtqttsslsaslgdrvtiscrasqdiskylnwyqqkpdgtvklliyhtsrlhsgvpsrfsgsgsgtdysltisnleqediatyfcqqgntlpytfg ggtkleitggggsggggsggggsevklgesgpglvapsqslsvtctvsgvslpdygvswirqpprkglewlgviwgsettyynsalksrltiik dnsksqvflkmnslqtddtaiyycakhyyyggsyamdywgqgtsvtvsstttpaprpptpaptiasqplslrpeacrpaaggavhtrgldfac diyiwaplagtcgvlllslvitlyckrgrkkllyifkqpfmrpvqttqeedgcscrfpeeeeggcelrvkfsrsadapaykqgqnqlynelnlgrr eeydvldkrrgrdpemggkprrknpgeglynelqkdkmaeayseigmkgerrrgkghdglygglstatkdtydalhmqalppr (SEQ ID NO: 1054), or a sequence substantially homologous thereto.

In some embodiments, the CAR molecule is a humanized CD19 CAR comprising the amino acid sequence of:

(SEQ ID NO: 3202)
MALPVTALLLPLALLLHAARPEIVMTQSPATLSLSPGERATLSCRASQD
ISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQL
QESGPGLVKPSETLSLTCTVSGVSLPDYGVSWIRQPPGKGLEWIGVIWG
SETTYYQSSLKSRVTISKDNSKNQVSLKLSSVTAADTAVYYCAKHYYYG
GSYAMDYWGQGTLVTVSSTTTPAPRPPTPAPTIASQPLSLRPEACRPAA
GGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKM
AEAYSEIGMKGERRRGKGHDGLYQGLSTATKDTYDALHMQALPPR

In some embodiments, the CAR molecule is a humanized CD19 CAR comprising the amino acid sequence of:

(SEQ ID NO: 3203)
EIVMTQSPATLSLSPGERATLSCRASQDISKYLNWYQQKPGQAPRLLIY
HTSRLHSGIPARFSGSGSGTDYTLTISSLQPEDFAVYFCQQGNTLPYTF
GQGTKLEIKGGGGSGGGGSGGGGSQVQLQESGPGLVKPSETLSLTCTVS
GVSLPDYGVSWIRQPPGKGLEWIGVIWGSETTYYQSSLKSRVTISKDNS
KNQVSLKLSSVTAADTAVYYCAKHYYYGGSYAMDYWGQGTLVTVSSTTT
PAPRPPTPAPTIASQPLSLRPEACRPAAGGAVHTRGLDFACDIYIWAPL
AGTCGVLLLSLVITLYCKRGRKKLLYIFKQPFMRPVQTTQEEDGCSCRF
PEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDG
LYQGLSTATKDTYDALHMQALPPR

In some embodiments, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In some embodiments, the CAR T cell that specifically binds to CD19 has the INN designation Axicabtagene ciloleucel. In one embodiment, the CAR T cell that specifically binds to CD19 has the USAN designation brexucabtagene autoleucel. In some embodiments, Axicabtagene ciloleucel is also known as YESCARTA®, Axi-cel, or KTE-C19. In some embodiments, brexucabtagene autoleucel is also known as KTE-X19 or TECARTUS®.

In one embodiment, the CAR T cell that specifically binds to CD19 has the INN designation Lisocabtagene maraleucel. In some embodiments, Lisocabtagene maraleucel is also known as JCAR017.

In other embodiments, the CD19 CAR comprises an antigen-binding domain (e.g., a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, CD19 CAR constructs are described in PCT publication WO 2012/079000, incorporated herein by reference, and the amino acid sequence of the murine CD19 CAR and scFv constructs are shown in Table 3 below, or a sequence substantially identical to any of the aforesaid sequences (e.g., at least 85%, 90%, 95% or more identical to any of the sequences described herein).

TABLE 3
Exemplary CD19 CAR Constructs

| | | |
|---|---|---|
| SEQ ID | | |
| NO | Region | Sequence |

CTL019

| | | |
|---|---|---|
| SEQ ID | CTL019 Full | MALPVTALLLPLALLLHAARPDIQMTQTTSSLSASLGDRVTISCR |
| NO: 1055 | amino acid | ASQDISKYLNWYQQKPDGTVKLLIYHTSRLHSGVPSRFSGSGSGT |
| | sequence | DYSLTISNLEQEDIATYFCQQGNTLPYTFGGGTKLEITGGGGSGG |
| | | GGSGGGGSEVKLQESGPGLVAPSQSLSVTCTVSGVSLPDYGVSWI |
| | | RQPPRKGLEWLGVIWGSETTYYNSALKSRLTIIKDNSKSQVFLKM |
| | | NSLQTDDTAIYYCAKHYYYGGSYAMDYWGQGTSVTVSSTTTPA |
| | | PRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDG |
| | | CSCRFPEEEEGGCELRVKFSRSADAPAYKQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |
| SEQ ID | CTL019 Full | ATGGCCTTACCAGTGACCGCCTTGCTCCTGCCGCTGGCCTTGC |
| NO: 1056 | nucleotide | TGCTCCACGCCGCCAGGCCGGACATCCAGATGACACAGACTA |
| | sequence | CATCCTCCCTGTCTGCCTCTCTGGGAGACAGAGTCACCATCAG |
| | | TTGCAGGGCAAGTCAGGACATTAGTAAATATTTAAATTGGTAT |
| | | CAGCAGAAACCAGATGGAACTGTTAAACTCCTGATCTACCAT |
| | | ACATCAAGATTACACTCAGGAGTCCCATCAAGGTTCAGTGGC |
| | | AGTGGGTCTGGAACAGATTATTCTCTCACCATTAGCAACCTGG |
| | | AGCAAGAAGATATTGCCACTTACTTTTGCCAACAGGGTAATAC |
| | | GCTTCCGTACACGTTCGGAGGGGGGACCAAGCTGGAGATCAC |
| | | AGGTGGCGGTGGCTCGGGCGGTGGTGGGTCGGGTGGCGGCGG |
| | | ATCTGAGGTGAAACTGCAGGAGTCAGGACCTGGCCTGGTGGC |
| | | GCCCTCACAGAGCCTGTCCGTCACATGCACTGTCTCAGGGGTC |
| | | TCATTACCCGACTATGGTGTAAGCTGGATTCGCCAGCCTCCAC |
| | | GAAAGGGTCTGGAGTGGCTGGGAGTAATATGGGGTAGTGAAA |
| | | CCACATACTATAATTCAGCTCTCAAATCCAGACTGACCATCAT |
| | | CAAGGACAACTCCAAGAGCCAAGTTTTCTTAAAAATGAACAG |
| | | TCTGCAAACTGATGACACAGCCATTTACTACTGTGCCAAACAT |
| | | TATTACTACGGTGGTAGCTATGCTATGGACTACTGGGGCCAAG |
| | | GAACCTCAGTCACCGTCTCCTCAACCACGACGCCAGCGCCGCG |
| | | ACCACCAACACCGGCGCCCACCATCGCGTCGCAGCCCCTGTCC |
| | | CTGCGCCCAGAGGCGTGCCGGCCAGCGGCGGGGGGCGCAGTG |
| | | CACACGAGGGGGCTGGACTTCGCCTGTGATATCTACATCTGGG |
| | | CGCCCTTGGCCGGGACTTGTGGGGTCCTTCTCCTGTCACTGGT |
| | | TATCACCCTTTACTGCAAACGGGGCAGAAAGAAACTCCTGTAT |
| | | ATATTCAAACAACCATTTATGAGACCAGTACAAACTACTCAAG |
| | | AGGAAGATGGCTGTAGCTGCCGATTTCCAGAAGAAGAAGAAG |
| | | GAGGATGTGAACTGAGAGTGAAGTTCAGCAGGAGCGCAGACG |
| | | CCCCCGCGTACAAGCAGGGCCAGAACCAGCTCTATAACGAGC |
| | | TCAATCTAGGACGAAGAGAGGAGTACGATGTTTTGGACAAGA |
| | | GACGTGGCCGGGACCCTGAGATGGGGGGAAAGCCGAGAAGG |
| | | AAGAACCCTCAGGAAGGCCTGTACAATGAACTGCAGAAAGAT |
| | | AAGATGGCGGAGGCCTACAGTGAGATTGGGATGAAAGGCGAG |
| | | CGCCGGAGGGGCAAGGGGCACGATGGCCTTTACCAGGGTCTC |
| | | AGTACAGCCACCAAGGACACCTACGACGCCCTTCACATGCAG |
| | | GCCCTGCCCCCTCGC |
| SEQ ID | CTL019 | DIQMTQTTSSLSASLGDRVTISCRASQDISKYLNWYQQKPDGTVK |
| NO: 1057 | scFv domain | LLIYHTSRLHSGVPSRFSGSGSGTDYSLTISNLEQEDIATYFCQQG |
| | | NTLPYTFGGGTKLEITGGGGSGGGGSGGGGSEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYG |
| | | GSYAMDYWGQGTSVTVSS |

mCAR1

| | | |
|---|---|---|
| SEQ ID | mCAR1 | QVQLLESGAELVRPGSSVKISCKASGYAFSSYWMNWVKQRPGQ |
| NO: 1058 | scFv | GLEWIGQIYPGDGDTNYNGKFKGQATLTADKSSSTAYMQLSGLT |
| | | SEDSAVYSCARKTISSVVDFYFDYWGQGTTVTGGGSGGGSGGGS |
| | | GGGSELVLTQSPKFMSTSVGDRVSVTCKASQNVGTNVAWYQQK |
| | | PGQSPKPLIYSATYRNSGVPDRFTGSGSGTDFTLTITNVQSKDLAD |
| | | YFCQYNRYPYTSFFFTKLEIKRRS |
| SEQ ID | mCAR1 Full | QVQLLESGAELVRPGSSVKISCKASGYAFSSYWMNWVKQRPGQ |
| NO: 1059 | amino acid | GLEWIGQIYPGDGDTNYNGKFKGQATLTADKSSSTAYMQLSGLT |
| | sequence | SEDSAVYSCARKTISSVVDFYFDYWGQGTTVTGGGSGGGSGGGS |
| | | GGGSELVLTQSPKFMSTSVGDRVSVTCKASQNVGTNVAWYQQK |
| | | PGQSPKPLIYSATYRNSGVPDRFTGSGSGTDFTLTITNVQSKDLAD |
| | | YFCQYNRYPYTSFFFTKLEIKRRSKIEVMYPPPYLDNEKSNGTIIH |
| | | VKGKHLCPSPLFPGPSKPFWVLVVVGGVLACYSLLVTVAFIIFWV |
| | | RSKRSRLLHSDYMNMTPRRPGPTRKHYQPYAPPRDFAAYRSRVK |
| | | FSRSADAPAYQQGQNQLYNELNLGRREEYDVLDKRRGRDPEMG |
| | | GKPRRKNPQEGLYNELQKDKMAEAYSEIGMKGERRRGKGHDGL |
| | | YQGLSTATKDTYDALHMQALPPR |

mCAR2

| | | |
|---|---|---|
| SEQ ID | mCAR2 | DIQMTQTTSSLSASLGDRVTISCRASQDISKYLNWYQQKPDGTVK |
| NO: 1060 | scFv | LLIYHTSRLHSGVPSRFSGSGSGTDYSLTISNLEQEDIATYFCQQG |
| | | NTLPYTFGGGTKLEITGSTSGSGKPGSGEGSTKGEVKLQESGPGL |
| | | VAPSQSLSVTCTVSGVSLPDYGVSWIRQPPRKGLEWLGVIWGSET |
| | | TYYNSALKSRLTIIKDNSKSQVFLKMNSLQTDDTAIYYCAKHYY |
| | | YGGSYAMDYWGQGTSVTVSSE |
| SEQ ID | mCAR2 | DIQMTQTTSSLSASLGDRVTISCRASQDISKYLNWYQQKPDGTVK |
| NO: 1061 | amino acid | LLIYHTSRLHSGVPSRFSGSGSGTDYSLTISNLEQEDIATYFCQQG |
| | sequence | NTLPYTFGGGTKLEITGSTSGSGKPGSGEGSTKGEVKLQESGPGL |
| | | VAPSQSLSVTCTVSGVSLPDYGVSWIRQPPRKGLEWLGVIWGSET |
| | | TYYNSALKSRLTIIKDNSKSQVFLKMNSLQTDDTAIYYCAKHYY |
| | | YGGSYAMDYWGQGTSVTVSSESKYGPPCPPCPMFWVLVVVGG |
| | | VLACYSLLVTVAFIIFWVKRGRKKLLYIFKQPFMRPVQTTQEEDG |
| | | CSCRFEEEEGGCELRVKFSRSADAPAYQQGQNQLYNELNLGRRE |
| | | EYDVLDKRRGRDPEMGGKPRRKNPQEGLYNELQKDKMAEAYS |
| | | EIGMKGERRRGKGHDGLYQGLSTATKDTYDALHMQALPPRL |
| SEQ ID | mCAR2 full | DIQMTQTTSSLSASLGDRVTISCRASQDISKYLNWYQQKPDGTVK |
| NO: 1062 | amino acid | LLIYHTSRLHSGVPSRFSGSGSGTDYSLTISNLEQEDIATYFCQQG |
| | sequence | NTLPYTFGGGTKLEITGSTSGSGKPGSGEGSTKGEVKLQESGPGL |
| | | VAPSQSLSVTCTVSGVSLPDYGVSWIRQPPRKGLEWLGVIWGSET |
| | | TYYNSALKSRLTIIKDNSKSQVFLKMNSLQTDDTAIYYCAKHYY |
| | | YGGSYAMDYWGQGTSVTVSSESKYGPPCPPCPMFWVLVVVGG |
| | | VLACYSLLVTVAFIIFWVKRGRKKLLYIFKQPFMRPVQTTQEEDG |
| | | CSCRFEEEEGGCELRVKFSRSADAPAYQQGQNQLYNELNLGRRE |
| | | EYDVLDKRRGRDPEMGGKPRRKNPQEGLYNELQKDKMAEAYS |
| | | EIGMKGERRRGKGHDGLYQGLSTATKDTYDALHMQALPPRLEG |
| | | GGEGRGSLLTCGDVEENPGPRMLLLVTSLLLCELPHPAFLLIPRK |
| | | VCNGIGIGEFKDSLSINATNIKHFKNCTSISGDLHILPVAFRGDSFT |
| | | HTPPLDPQELDILKTVKEITGFLLIQAWPENRTDLHAFENLEIIRGR |
| | | TKQHGQFSLAVVSLNITSLGLRSLKEISDGDVIISGNKNLCYANTI |
| | | NWKKLFGTSGQKTKIISNRGENSCKATGQVCHALCSPEGCWGPE |
| | | PRDCVSCRNVSRGRECVDKCNLLEGEPREFVENSECIQCHPECLP |
| | | QAMNITCTGRGPDNCIQCAHYIDGPHCVKTCPAGVMGENNTLV |
| | | WKYADAGHVCHLCHPNCTYGCTGPGLEGCPTNGPKIPSIATGMV |
| | | GALLLLLVVALGIGLFM |

mCAR3

| | | |
|---|---|---|
| SEQ ID | mCAR3 | DIQMTQTTSSLSASLGDRVTISCRASQDISKYLNWYQQKPDGTVK |
| NO: 1063 | scFv | LLIYHTSRLHSGVPSRFSGSGSGTDYSLTISNLEQEDIATYFCQQG |
| | | NTLPYTFGGGTKLEITGSTSGSGKPGSGEGSTKGEVKLQESGPGL |
| | | VAPSQSLSVTCTVSGVSLPDYGVSWIRQPPRKGLEWLGVIWGSET |
| | | TYYNSALKSRLTIIKDNSKSQVFLKMNSLQTDDTAIYYCAKHYY |
| | | YGGSYAMDYWGQGTSVTVSS |
| SEQ ID | mCAR3 full | DIQMTQTTSSLSASLGDRVTISCRASQDISKYLNWYQQKPDGTVK |
| NO: 1064 | amino acid | LLIYHTSRLHSGVPSRFSGSGSGTDYSLTISNLEQEDIATYFCQQG |
| | sequence | NTLPYTFGGGTKLEITGSTSGSGKPGSGEGSTKGEVKLQESGPGL |
| | | VAPSQSLSVTCTVSGVSLPDYGVSWIRQPPRKGLEWLGVIWGSET |
| | | TYYNSALKSRLTIIKDNSKSQVFLKMNSLQTDDTAIYYCAKHYY |
| | | YGGSYAMDYWGQGTSVTVSSAAAIEVMYPPPYLDNEKSNGTIIH |
| | | VKGKHLCPSPLFPGPSKPFWVLVVVGGVLACYSLLVTVAFIIFWV |
| | | RSKRSRLLHSDYMNMTPRRPGPTRKHYQPYAPPRDFAAYRSRVK |
| | | FSRSADAPAYQQGQNQLYNELNLGRREEYDVLDKRRGRDPEMG |
| | | GKPRRKNPQEGLYNELQKDKMAEAYSEIGMKGERRRGKGHDGL |
| | | YQGLSTATKDTYDALHMQALPPR |

SSJ25-C1

| | | |
|---|---|---|
| SEQ ID | SSJ25-C1 | QVQLLESGAELVRPGSSVKISCKASGYAFSSYWMNWVKQRPGQ |
| NO: 1065 | VH sequence | GLEWIGQIYPGDGDTNYNGKFKGQATLTADKSSSTAYMQLSGLT |
| | | SEDSAVYSCARKTISSVVDFYFDYWGQGTTVT |
| SEQ ID | SSJ25-C1 | ELVLTQSPKFMSTSVGDRVSVTCKASQNVGTNVAWYQQKPGQS |
| NO: 1066 | VL | PKPLIYSATYRNSGVPDRFTGSGSGTDFTLTITNVQSKDLADYFYF |
| | | CQYNRYPYTSGGGTKLEIKRRS |

Humanized CAR1

| | | |
|---|---|---|
| SEQ ID | CAR1 scFv | EIVMTQSPATLSLSPGERATLSCRASQDISKYLNWYQQKPGQAPR |
| NO: 1067 | domain | LLIYHTSRLHSGIPARFSGSGSGTDYTLTISSLQPEDFAVYFCQQG |
| | | NTLPYTFGQGTKLEIKGGGGSGGGGSGGGGSQVQLQESGPGLVK |
| | | PSETLSLTCTVSGVSLPDYGVSWIRQPPGKGLEWIGVIWGSETTY |
| | | YSSSLKSRVTISKDNSKNQVSLKLSSVTAADTAVYYCAKHYYYG |
| | | GSYAMDYWGQGTLVTVSS |
| SEQ ID | CAR 1-Full- | MALPVTALLLPLALLLHAARPEIVMTQSPATLSLSPGERATLSCR |
| NO: 1068 | aa | ASQDISKYLNWYQQKPGQAPRLLIYHTSRLHSGIPARFSGSGSGT |
| | | DYTLTISSLQPEDFAVYFCQQGNTLPYTFGQGTKLEIKGGGGSGG |
| | | GGSGGGGSQVQLQESGPGLVKPSETLSLTCTVSGVSLPDYGVSWI |
| | | RQPPGKGLEWIGVIWGSETTYYSSSLKSRVTISKDNSKNQVSLKL |
| | | SSVTAADTAVYYCAKHYYYGGSYAMDYWGQGTLVTVSSTTTP |
| | | APRPPTPAPTIASQPLSLRPEACRPAAGGAVHTRGLDFACDIYIWA |
| | | PLAGTCGVLLLSLVITLYCKRGRKKLLYIFKQPFMRPVQTTQEED |
| | | GCSCRFPEEEEGGCELRVKFSRSADAPAYKQGQNQLYNELNLGR |
| | | REEYDVLDKRRGRDPEMGGKPRRKNPQEGLYNELQKDKMAEA |
| | | YSEIGMKGERRRGKGHDGLYQGLSTATKDTYDALHMQALPPR |

Humanized CAR2

| | | |
|---|---|---|
| SEQ ID | CAR2 scFv | EIVMTQSPATLSLSPGERATLSCRASQDISKYLNWYQQKPGQAPR |
| NO: 1069 | domain-aa | LLIYHTSRLHSGIPARFSGSGSGTDYTLTISSLQPEDFAVYFCQQG |
| | (Linker is | NTLPYTFGQGTKLEIKGGGGSGGGGSGGGGSQVQLQESGPGLVK |
| | underlined) | PSETLSLTCTVSGVSLPDYGVSWIRQPPGKGLEWIGVIWGSETTY |
| | | YQSSLKSRVTISKDNSKNQVSLKLSSVTAADTAVYYCAKHYYYG |
| | | GSYAMDYWGQGTLVTVSS |
| SEQ ID | CAR2 scFv | atggccctccctgtcaccgccctgctgcttccgctggctcttctgctccacgccgctcggcccgaaattg |
| NO: 1070 | domain-nt | tgatgacccagtcacccgccactcttagcctttcacccggtgagcgcgcaaccctgtcttgcagagcct |
| | | cccaagacatctcaaaataccttaattggtatcaacagaagcccggacaggctcctcgccttctgatcta |
| | | ccacaccagccggctccattctggaatccctgccaggttcagcggtagcggatctgggaccgactaca |
| | | ccctcactatcagctcactgcagccagaggacttcgctgtctatttctgtcagcaagggaacaccctgcc |
| | | ctacacctttggacagggcaccaagctcgagattaaaggtggaggtggcagcggaggaggtgggtcc |
| | | ggcggtggaggaagccaggtccaactccaagaaagcggaccgggtcttgtgaagccatcagaaact |
| | | ctttcactgacttgtactgtgagcggagtgtctctccccgattacggggtgtcttggatcagacagccacc |
| | | ggggaagggtctggaatggattggagtgatttggggctctgagactacttactaccaatcatccctcaag |
| | | tcacgcgtcaccatctcaaaggacaactctaagaatcaggtgtcactgaaactgtcatctgtgaccgcag |
| | | ccgacaccgccgtgtactattgcgctaagcattactattatggcgggagctacgcaatggattactgggg |
| | | acagggtactctggtcaccgtgtccagccaccaccatcatcaccatcaccat |
| SEQ ID | CAR 2-Full- | MALPVTALLLPLALLLHAARPEIVMTQSPATLSLSPGERATLSCR |
| NO: 1071 | aa | ASQDISKYLNWYQQKPGQAPRLLIYHTSRLHSGIPARFSGSGSGT |
| | | DYTLTISSLQPEDFAVYFCQQGNTLPYTFGQGTKLEIKGGGGSGG |
| | | GGSGGGGSQVQLQESGPGLVKPSETLSLTCTVSGVSLPDYGVSWI |
| | | RQPPGKGLEWIGVIWGSETTYYQSSLKSRVTISKDNSKNQVSLKL |
| | | SSVTAADTAVYYCAKHYYYGGSYAMDYWGQGTLVTVSSTTTP |
| | | APRPPTPAPTIASQPLSLRPEACRPAAGGAVHTRGLDFACDIYIWA |
| | | PLAGTCGVLLLSLVITLYCKRGRKKLLYIFKQPFMRPVQTTQEED |
| | | GCSCRFPEEEEGGCELRVKFSRSADAPAYKQGQNQLYNELNLGR |
| | | REEYDVLDKRRGRDPEMGGKPRRKNPQEGLYNELQKDKMAEA |
| | | YSEIGMKGERRRGKGHDGLYQGLSTATKDTYDALHMQALPPR |
| SEQ ID | CAR 2- | EIVMTQSPATLSLSPGERATLSCRASQDISKYLNWYQQKPGQAPR |
| NO: 3207 | Full-aa; no | LLIYHTSRLHSGIPARFSGSGSGTDYTLTISSLQPEDFAVYFCQQG |
| | leader | NTLPYTFGQGTKLEIKGGGGSGGGGSGGGGSQVQLQESGPGLVK |
| | | PSETLSLTCTVSGVSLPDYGVSWIRQPPGKGLEWIGVIWGSETTY |
| | | YQSSLKSRVTISKDNSKNQVSLKLSSVTAADTAVYYCAKHYYYG |
| | | GSYAMDYWGQGTLVTVSSTTTPAPRPPTPAPTIASQPLSLRPEAC |
| | | RPAAGGAVHTRGLDFACDIYIWAPLAGTCGVLLLSLVITLYCKR |
| | | GRKKLLYIFKQPFMRPVQTTQEEDGCSCRFPEEEEGGCELRVKFS |
| | | RSADAPAYKQGQNQLYNELNLGRREEYDVLDKRRGRDPEMGG |
| | | KPRRKNPQEGLYNELQKDKMAEAYSEIGMKGERRRGKGHDGLY |
| | | QGLSTATKDTYDALHMQALPPR |
| SEQ ID | CAR 2-Full- | atggccctccctgtcaccgccctgctgcttccgctggctcttctgctccacgccgctcggcccgaaattg |
| NO: 1072 | nt | tgatgacccagtcacccgccactcttagcctttcacccggtgagcgcgcaaccctgtcttgcagagcct |
| | | cccaagacatctcaaaataccttaattggtatcaacagaagcccggacaggctcctcgccttctgatcta |
| | | ccacaccagccggctccattctggaatccctgccaggttcagcggtagcggatctgggaccgactaca |
| | | ccctcactatcagctcactgcagccagaggacttcgctgtctatttctgtcagcaagggaacaccctgcc |
| | | ctacacctttggacagggcaccaagctcgagattaaaggtggaggtggcagcggaggaggtgggtcc |
| | | ggcggtggaggaagccaggtccaactccaagaaagcggaccgggtcttgtgaagccatcagaaact |
| | | ctttcactgacttgtactgtgagcggagtgtctctccccgattacggggtgtcttggatcagacagccacc |
| | | ggggaagggtctggaatggattggagtgatttggggctctgagactacttactaccaatcatccctcaag |
| | | tcacgcgtcaccatctcaaaggacaactctaagaatcaggtgtcactgaaactgtcatctgtgaccgcag |
| | | ccgacaccgccgtgtactattgcgctaagcattactattatggcgggagctacgcaatggattactgggg |
| | | acagggtactctggtcaccgtgtccagcaccactaccccagcaccgaggccacccaccccggctcct |
| | | accatcgcctcccagcctctgtccctgcgtccggaggcatgtagacccgcagctggtggggccgtgca |
| | | tacccggggtcttgacttcgcctgcgatatctacatttgggcccctctggctggtacttgcggggtcctgc |
| | | tgctttcactcgtgatcactctttactgtaagcgcggtcggaagaagctgctgtacatctttaagcaaccc |
| | | ttcatgaggcctgtgcagactactcaagaggaggacggctgttcatgccggttcccagaggaggagga |
| | | aggcggctgcgaactgcgcgtgaaattcagccgcagcgcagatgctccagcctacaagcaggggca |
| | | gaaccagctctacaacgaactcaatcttggtcggagagaggagtacgacgtgctggacaagcggaga |
| | | ggacgggacccagaaatgggcgggaagccgcgcagaaagaatccccaagagggcctgtacaacg |
| | | agctccaaaaggataagatggcagaagcctatagcgagattggtatgaaaggggaacgcagaagag |
| | | gcaaaggccacgacggactgtaccagggactcagcaccgccaccaaggacacctatgacgctcttca |
| | | catgcaggccctgccgcctcgg |
| SEQ ID | CAR 2- | gaaattgtgatgacccagtcacccgccactcttagcctttcacccggtgagcgcgcaaccctgtcttgca |
| NO: 3208 | Full-nt; no | gagcctcccaagacatctcaaaataccttaattggtatcaacagaagcccggacaggctcctcgccttct |
| | leader | gatctaccacaccagccggctccattctggaatccctgccaggttcagcggtagcggatctgggaccg |
| | | actacaccctcactatcagctcactgcagccagaggacttcgctgtctatttctgtcagcaagggaacac |
| | | cctgccctacacctttggacagggcaccaagctcgagattaaaggtggaggtggcagcggaggaggt |
| | | gggtccggcggtggaggaagccaggtccaactccaagaaagcggaccgggtcttgtgaagccatca |
| | | gaaactctttcactgacttgtactgtgagcggagtgtctctccccgattacggggtgtcttggatcagaca |
| | | gccaccggggaagggtctggaatggattggagtgatttggggctctgagactacttactaccaatcatc |
| | | cctcaagtcacgcgtcaccatctcaaaggacaactctaagaatcaggtgtcactgaaactgtcatctgtg |
| | | accgcagccgacaccgccgtgtactattgcgctaagcattactattatggcgggagctacgcaatggat |
| | | tactggggacagggtactctggtcaccgtgtccagcaccactaccccagcaccgaggccacccaccc |
| | | cggctcctaccatcgcctcccagcctctgtccctgcgtccggaggcatgtagacccgcagctggtggg |
| | | gccgtgcatacccggggtcttgacttcgcctgcgatatctacatttgggcccctctggctggtacttgcgg |
| | | ggtcctgctgctttcactcgtgatcactctttactgtaagcgcggtcggaagaagctgctgtacatctttaa |
| | | gcaacccttcatgaggcctgtgcagactactcaagaggaggacggctgttcatgccggttcccagagg |
| | | aggaggaaggggctgcgaactgcgcgtgaaattcagccgcagcgcagatgctccagcctacaagc |
| | | aggggcagaaccagctctacaacgaactcaatcttggtcggagagaggagtacgacgtgctggacaa |
| | | gcggagaggacgggacccagaaatgggcgggaagccgcgcagaaagaatccccaagagggcctg |
| | | tacaacgagctccaaaaggataagatggcagaagcctatagcgagattggtatgaaaggggaacgca |
| | | gaagaggcaaaggccacgacggactgtaccagggactcagcaccgccaccaaggacacctatgac |
| | | gctcttcacatgcaggccctgccgcctcgg |
| SEQ ID | CAR 2- | atggccctccctgtcaccgccctgctgcttccgctggctcttctgctccacgc |
| NO: 3209 | Full-nt with | cgctcggcccgaaattgtgatgacccagtcacccgccactcttagcctttcac |
| | hairpin | ccggtgagcgcgcaaccctgtcttgcagagcctcccaagacatctcaaaatac |
| | modification | cttaattggtatcaacagaagcccggacaggctcctcgccttctgatctacca |
| | | caccagccggctccattctggaatccctgccaggttcagcggtagcggatctg |
| | | ggaccgactacaccctcactatcagctcactgcagccagaggacttcgctgtc |
| | | tatttctgtcagcaagggaacaccctgccctacacctttggacagggcaccaa |
| | | gctcgagattaaaggtggaggtggcagcggaggaggtgggtccggcggtggag |
| | | gaagccaggtccaactccaagaaagcggaccgggtcttgtgaagccatcagaa |
| | | actctttcactgacttgtactgtgagcggagtgtctctccccgattacggggt |
| | | gtcttggatcagacagccaccggggaagggtctggaatggattggagtgattt |
| | | ggggctcAgagactacttactaccaatcatccctcaagtcTcgcgtcaccatc |
| | | tcaaaggacaactctaagaatcaggtgtcactgaaactgtcatctgtgaccgc |
| | | agccgacaccgccgtgtactattgcgctaagcattactattatggcgggagct |
| | | acgcaatggattactggggacagggtactctggtcaccgtgtccagcaccact |
| | | accccagcaccgaggccacccaccccggctcctaccatcgcctcccagcctct |
| | | gtccctgcgtccggaggcatgtagacccgcagctggtggggccgtgcataccc |
| | | ggggtcttgacttcgcctgcgatatctacatttgggcccctctggctggtact |
| | | tgcggggtcctgctgctttcactcgtgatcactctttactgtaagcgcggtcg |
| | | gaagaagctgctgtacatctttaagcaacccttcatgaggcctgtgcagacta |
| | | ctcaagaggaggacggctgttcatgccggttcccagaggaggaggaaggcggc |
| | | tgcgaactgcgcgtgaaattcagccgcagcgcagatgctccagcctacAagca |
| | | ggggcagaaccagctctacaacgaactcaatcttggtcggagagaggagtacg |
| | | acgtgctggacaagcggagaggacgggacccagaaatgggcgggaagccgcgc |
| | | agaaagaatccccaagagggcctgtacaacgagctccaaaaggataagatggc |
| | | agaagcctatagcgagattggtatgaaaggggaacgcagaagaggcaaaggcc |
| | | acgacggactgtaccagggactcagcaccgccaccaaggacacctatgacgct |
| | | cttcacatgcaggccctgccgcctcgg |
| SEQ ID | CAR 2- | gaaattgtgatgacccagtcacccgccactcttagcctttcacccggtgagcg |
| NO: 3210 | Full-nt with | cgcaaccctgtcttgcagagcctcccaagacatctcaaaataccttaattggt |
| | hairpin | atcaacagaagcccggacaggctcctcgccttctgatctaccacaccagccgg |
| | modification | ctccattctggaatccctgccaggttcagcggtagcggatctgggaccgacta |
| | and no leader | caccctcactatcagctcactgcagccagaggacttcgctgtctatttctgtc |
| | | agcaagggaacaccctgccctacacctttggacagggcaccaagctcgagatt |
| | | aaaggtggaggtggcagcggaggaggtgggtccggcggtggaggaagccaggt |
| | | ccaactccaagaaagcggaccgggtcttgtgaagccatcagaaactctttcac |
| | | tgacttgtactgtgagcggagtgtctctccccgattacggggtgtcttggatc |
| | | agacagccaccggggaagggtctggaatggattggagtgatttggggctcAga |
| | | gactacttactaccaatcatccctcaagtcTcgcgtcaccatctcaaaggaca |
| | | actctaagaatcaggtgtcactgaaactgtcatctgtgaccgcagccgacacc |
| | | gccgtgtactattgcgctaagcattactattatggcgggagctacgcaatgga |
| | | ttactggggacagggtactctggtcaccgtgtccagcaccactaccccagcac |
| | | cgaggccacccaccccggctcctaccatcgcctcccagcctctgtccctgcgt |
| | | ccggaggcatgtagacccgcagctggtggggccgtgcatacccggggtcttga |
| | | cttcgcctgcgatatctacatttgggcccctctggctggtacttgcggggtcc |
| | | tgctgctttcactcgtgatcactctttactgtaagcgcggtcggaagaagctg |
| | | ctgtacatctttaagcaacccttcatgaggcctgtgcagactactcaagagga |
| | | ggacggctgttcatgccggttcccagaggaggaggaaggcggctgcgaactgc |
| | | gcgtgaaattcagccgcagcgcagatgctccagcctacAagcaggggcagaac |
| | | cagctctacaacgaactcaatcttggtcggagagaggagtacgacgtgctgga |
| | | caagcggagaggacgggacccagaaatgggcgggaagccgcgcagaaagaatc |
| | | cccaagagggcctgtacaacgagctccaaaaggataagatggcagaagcctat |
| | | agcgagattggtatgaaaggggaacgcagaagaggcaaaggccacgacggact |
| | | gtaccagggactcagcaccgccaccaaggacacctatgacgctcttcacatgc |
| | | aggccctgccgcctcgg |
| SEQ ID | CAR2- | MALPVTALLLPLALLLHAARPeivmtqspatlslspgeratlscrasqdiskylnw |
| NO: 1073 | Soluble scFv- | yqqkpgqaprlliyhtsrlhsgiparfsgsgsgtdytltisslqpedfavyfcqqgntlpytfgqgtklei |
| | aa | kggggsggggsggggsqvqlqesgpglvkpsetlsltctvsgvslpdygvswirqppgkglewig |
| | | viwgsettyyqsslksrvtiskdnsknqvslklssvtaadtavyycakhyyyggsyamdywgqgtl |
| | | vtvsshhhhhhhh |
| SEQ ID | CAR 2A- | MALPVTALLLPLALLLHAARPEIVMTQSPATLSLSPGERATLSCRASQDISKY |
| NO: 3202 | Full-aa; | LNWYQQKPGQAPRLLIYHTSRLHSGIPARFSGSGSGTDYTLTISSLQPEDFAV |
| | leader | YFCQQGNTLPYTFGQGTKLEIKGGGGSGGGGSGGGGSQVQLQESGPGLVKPSE |
| | underlined | TLSLTCTVSGVSLPDYGVSWIRQPPGKGLEWIGVIWGSETTYYQSSLKSRVTI |
| | | SKDNSKNQVSLKLSSVTAADTAVYYCAKHYYYGGSYAMDYWGQGTLVTVSSTT |
| | | TPAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKDTYDA |
| | | LHMQALPPR |
| SEQ ID | CAR 2A- | EIVMTQSPATLSLSPGERATLSCRASQDISKYLNWYQQKPGQAPRLLIYHTSR |
| NO: 3203 | Full-aa; no | LHSGIPARFSGSGSGTDYTLTISSLQPEDFAVYFCQQGNTLPYTFGQGTKLEI |
| | leader | KGGGGSGGGGSGGGGSQVQLQESGPGLVKPSETLSLTCTVSGVSLPDYGVSWI |
| | | RQPPGKGLEWIGVIWGSETTYYQSSLKSRVTISKDNSKNQVSLKLSSVTAADT |
| | | AVYYCAKHYYYGGSYAMDYWGQGTLVTVSSTTTPAPRPPTPAPTIASQPLSLR |
| | | PEACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAY |
| | | SEIGMKGERRRGKGHDGLYQGLSTATKDTYDALHMQALPPR |
| SEQ ID | CAR 2A- | atggccctccctgtcaccgccctgctgcttccgctggctcttctgctccacgccgctcggcccgaaattg |
| NO: 3211 | Full-nt; | tgatgacccagtcacccgccactcttagcctttcacccggtgagcgcgcaaccctgtcttgcagagcct |
| | leader | cccaagacatctcaaaataccttaattggtatcaacagaagcccggacaggctcctcgccttctgatcta |
| | underlined | ccacaccagccggctccattctggaatccctgccaggttcagcggtagcggatctgggaccgactaca |
| | | ccctcactatcagctcactgcagccagaggacttcgctgtctatttctgtcagcaagggaacaccctgcc |
| | | ctacacctttggacagggcaccaagctcgagattaaaggtggaggtggcagcggaggaggtgggtcc |
| | | ggcggtggaggaagccaggtccaactccaagaaagcggaccgggtcttgtgaagccatcagaaact |
| | | ctttcactgacttgtactgtgagcggagtgtctctccccgattacggggtgtcttggatcagacagccacc |
| | | ggggaagggtctggaatggattggagtgatttggggctctgagactacttactaccaatcatccctcaag |
| | | tcacgcgtcaccatctcaaaggacaactctaagaatcaggtgtcactgaaactgtcatctgtgaccgcag |
| | | ccgacaccgccgtgtactattgcgctaagcattactattatggcgggagctacgcaatggattactgggg |
| | | acagggtactctggtcaccgtgtccagcaccactaccccagcaccgaggccacccaccccggctcct |
| | | accatcgcctcccagcctctgtccctgcgtccggaggcatgtagacccgcagctggtggggccgtgca |
| | | tacccggggtcttgacttcgcctgcgatatctacatttgggcccctctggctggtacttgcggggtcctgc |
| | | tgctttcactcgtgatcactctttactgtaagcgcggtcggaagaagctgctgtacatctttaagcaaccct |
| | | tcatgaggcctgtgcagactactcaagaggaggacggctgttcatgccggttcccagaggaggagga |
| | | aggcggctgcgaactgcgcgtgaaattcagccgcagcgcagatgctccagcctaccagcaggggca |
| | | gaaccagctctacaacgaactcaatcttggtcggagagaggagtacgacgtgctggacaagcggaga |
| | | ggacgggacccagaaatgggcgggaagccgcgcagaaagaatccccaagagggcctgtacaacg |
| | | agctccaaaaggataagatggcagaagcctatagcgagattggtatgaaaggggaacgcagaagag |
| | | gcaaaggccacgacggactgtaccagggactcagcaccgccaccaaggacacctatgacgctcttca |
| | | catgcaggccctgccgcctcgg |
| SEQ ID | CAR 2A- | gaaattgtgatgacccagtcacccgccactcttagcctttcacccggtgagcgcgcaaccctgtcttgca |
| NO: 3212 | Full-nt; no | gagcctcccaagacatctcaaaataccttaattggtatcaacagaagcccggacaggctcctcgccttct |
| | leader | gatctaccacaccagccggctccattctggaatccctgccaggttcagcggtagcggatctgggaccg |
| | | actacaccctcactatcagctcactgcagccagaggacttcgctgtctatttctgtcagcaagggaacac |
| | | cctgccctacacctttggacagggcaccaagctcgagattaaaggtggaggtggcagcggaggaggt |
| | | gggtccggcggtggaggaagccaggtccaactccaagaaagcggaccgggtcttgtgaagccatca |
| | | gaaactctttcactgacttgtactgtgagcggagtgtctctccccgattacggggtgtcttggatcagaca |
| | | gccaccggggaagggtctggaatggattggagtgatttggggctctgagactacttactaccaatcatc |
| | | cctcaagtcacgcgtcaccatctcaaaggacaactctaagaatcaggtgtcactgaaactgtcatctgtg |
| | | accgcagccgacaccgccgtgtactattgcgctaagcattactattatggcgggagctacgcaatggat |
| | | tactggggacagggtactctggtcaccgtgtccagcaccactaccccagcaccgaggccacccaccc |
| | | cggctcctaccatcgcctcccagcctctgtccctgcgtccggaggcatgtagacccgcagctggtggg |
| | | gccgtgcatacccggggtcttgacttcgcctgcgatatctacatttgggcccctctggctggtacttgcgg |
| | | ggtcctgctgctttcactcgtgatcactctttactgtaagcgcggtcggaagaagctgctgtacatctttaa |
| | | gcaacccttcatgaggcctgtgcagactactcaagaggaggacggctgttcatgccggttcccagagg |
| | | aggaggaaggcggctgcgaactgcgcgtgaaattcagccgcagcgcagatgctccagcctaccagc |
| | | aggggcagaaccagctctacaacgaactcaatcttggtcggagagaggagtacgacgtgctggacaa |
| | | gcggagaggacgggacccagaaatgggcgggaagccgcgcagaaagaatccccaagagggcctg |
| | | tacaacgagctccaaaaggataagatggcagaagcctatagcgagattggtatgaaaggggaacgca |
| | | gaagaggcaaaggccacgacggactgtaccagggactcagcaccgccaccaaggacacctatgac |
| | | gctcttcacatgcaggccctgccgcctcgg |
| SEQ ID | CAR 2A- | atggccctccctgtcaccgccctgctgcttccgctggctcttctgctccacgc |
| NO: 3213 | Full-nt; | cgctcggcccgaaattgtgatgacccagtcacccgccactcttagcctttcac |
| | with hairpin | ccggtgagcgcgcaaccctgtcttgcagagcctcccaagacatctcaaaatac |
| | modification | cttaattggtatcaacagaagcccggacaggctcctcgccttctgatctacca |
| | and leader | caccagccggctccattctggaatccctgccaggttcagcggtagcggatctg |
| | underlined | ggaccgactacaccctcactatcagctcactgcagccagaggacttcgctgtc |
| | | tatttctgtcagcaagggaacaccctgccctacacctttggacagggcaccaa |
| | | gctcgagattaaaggtggaggtggcagcggaggaggtgggtccggcggtggag |
| | | gaagccaggtccaactccaagaaagcggaccgggtcttgtgaagccatcagaa |
| | | actctttcactgacttgtactgtgagcggagtgtctctccccgattacggggt |
| | | gtcttggatcagacagccaccggggaagggtctggaatggattggagtgattt |
| | | ggggctcAgagactacttactaccaatcatccctcaagtcTcgcgtcaccatc |
| | | tcaaaggacaactctaagaatcaggtgtcactgaaactgtcatctgtgaccgc |
| | | agccgacaccgccgtgtactattgcgctaagcattactattatggcgggagct |
| | | acgcaatggattactggggacagggtactctggtcaccgtgtccagcaccact |
| | | accccagcaccgaggccacccaccccggctcctaccatcgcctcccagcctct |
| | | gtccctgcgtccggaggcatgtagacccgcagctggtggggccgtgcataccc |
| | | ggggtcttgacttcgcctgcgatatctacatttgggcccctctggctggtact |
| | | tgcggggtcctgctgctttcactcgtgatcactctttactgtaagcgcggtcg |
| | | gaagaagctgctgtacatctttaagcaacccttcatgaggcctgtgcagacta |
| | | ctcaagaggaggacggctgttcatgccggttcccagaggaggaggaaggcggc |
| | | tgcgaactgcgcgtgaaattcagccgcagcgcagatgctccagcctacCagca |
| | | ggggcagaaccagctctacaacgaactcaatcttggtcggagagaggagtacg |
| | | acgtgctggacaagcggagaggacgggacccagaaatgggcgggaagccgcgc |
| | | agaaagaatccccaagagggcctgtacaacgagctccaaaaggataagatggc |
| | | agaagcctatagcgagattggtatgaaaggggaacgcagaagaggcaaaggcc |
| | | acgacggactgtaccagggactcagcaccgccaccaaggacacctatgacgct |
| | | cttcacatgcaggccctgccgcctcgg |
| SEQ ID | CAR 2A- | gaaattgtgatgacccagtcacccgccactcttagcctttcacccggtgagcg |
| NO: 3214 | Full-nt; | cgcaaccctgtcttgcagagcctcccaagacatctcaaaataccttaattggt |
| | with hairpin | atcaacagaagcccggacaggctcctcgccttctgatctaccacaccagccgg |
| | modification | ctccattctggaatccctgccaggttcagcggtagcggatctgggaccgacta |
| | and no leader | caccctcactatcagctcactgcagccagaggacttcgctgtctatttctgtc |
| | | agcaagggaacaccctgccctacacctttggacagggcaccaagctcgagatt |
| | | aaaggtggaggtggcagcggaggaggtgggtccggcggtggaggaagccaggt |
| | | ccaactccaagaaagcggaccgggtcttgtgaagccatcagaaactctttcac |
| | | tgacttgtactgtgagcggagtgtctctccccgattacggggtgtcttggatc |
| | | agacagccaccggggaagggtctggaatggattggagtgatttggggctcAga |
| | | gactacttactaccaatcatccctcaagtcTcgcgtcaccatctcaaaggaca |
| | | actctaagaatcaggtgtcactgaaactgtcatctgtgaccgcagccgacacc |
| | | gccgtgtactattgcgctaagcattactattatggcgggagctacgcaatgga |
| | | ttactggggacagggtactctggtcaccgtgtccagcaccactaccccagcac |
| | | cgaggccacccaccccggctcctaccatcgcctcccagcctctgtccctgcgt |
| | | ccggaggcatgtagacccgcagctggtggggccgtgcatacccggggtcttga |
| | | cttcgcctgcgatatctacatttgggcccctctggctggtacttgcggggtcc |
| | | tgctgctttcactcgtgatcactctttactgtaagcgcggtcggaagaagctg |
| | | ctgtacatctttaagcaacccttcatgaggcctgtgcagactactcaagagga |
| | | ggacggctgttcatgccggttcccagaggaggaggaaggcggctgcgaactgc |
| | | gcgtgaaattcagccgcagcgcagatgctccagcctacCagcaggggcagaac |
| | | cagctctacaacgaactcaatcttggtcggagagaggagtacgacgtgctgga |
| | | caagcggagaggacgggacccagaaatgggcgggaagccgcgcagaaagaatc |
| | | cccaagagggcctgtacaacgagctccaaaaggataagatggcagaagcctat |
| | | agcgagattggtatgaaaggggaacgcagaagaggcaaaggccacgacggact |
| | | gtaccagggactcagcaccgccaccaaggacacctatgacgctcttcacatgc |
| | | aggccctgccgcctcgg |

Humanized CAR3

| | | |
|---|---|---|
| SEQ ID | CAR3 scFv | QVQLQESGPGLVKPSETLSLTCTVSGVSLPDYGVSWIRQPPGKGL |
| NO: 1074 | domain | EWIGVIWGSETTYYSSSLKSRVTISKDNSKNQVSLKLSSVTAADT |
| | | AVYYCAKHYYYGGSYAMDYWGQGTLVTVSSGGGGSGGGGSG |
| | | GGGSEIVMTQSPATLSLSPGERATLSCRASQDISKYLNWYQQKPG |
| | | QAPRLLIYHTSRLHSGIPARFSGSGSGTDYTLTISSLQPEDFAVYFC |
| | | QQGNTLPYTFGQGTKLEIK |
| SEQ ID | CAR 3-Full- | MALPVTALLLPLALLLHAARPQVQLQESGPGLVKPSETLSLTCTV |
| NO: 1075 | aa | SGVSLPDYGVSWIRQPPGKGLEWIGVIWGSETTYYSSSLKSRVTIS |
| | | KDNSKNQVSLKLSSVTAADTAVYYCAKHYYYGGSYAMDYWGQ |
| | | GTLVTVSSGGGGSGGGGSGGGGSEIVMTQSPATLSLSPGERATLS |
| | | CRASQDISKYLNWYQQKPGQAPRLLIYHTSRLHSGIPARFSGSGS |
| | | GTDYTLTISSLQPEDFAVYFCQQGNTLPYTFGQGTKLEIKTTTPAP |
| | | RPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGC |
| | | SCRFPEEEEGGCELRVKFSRSADAPAYKQGQNQLYNELNLGRRE |
| | | EYDVLDKRRGRDPEMGGKPRRKNPQEGLYNELQKDKMAEAYS |
| | | EIGMKGERRRGKGHDGLYQGLSTATKDTYDALHMQALPPR |

Humanized CAR4

| | | |
|---|---|---|
| SEQ ID | CAR4 scFv | QVQLQESGPGLVKPSETLSLTCTVSGVSLPDYGVSWIRQPPGKGL |
| NO: 1076 | domain | EWIGVIWGSETTYYQSSLKSRVTISKDNSKNQVSLKLSSVTAADT |
| | | AVYYCAKHYYYGGSYAMDYWGQGTLVTVSSGGGGSGGGGSG |
| | | GGGSEIVMTQSPATLSLSPGERATLSCRASQDISKYLNWYQQKPG |
| | | QAPRLLIYHTSRLHSGIPARFSGSGSGTDYTLTISSLQPEDFAVYFC |
| | | QQGNTLPYTFGQGTKLEIK |
| SEQ ID | CAR 4-Full- | MALPVTALLLPLALLLHAARPQVQLQESGPGLVKPSETLSLTCTV |
| NO: 1077 | aa | SGVSLPDYGVSWIRQPPGKGLEWIGVIWGSETTYYQSSLKSRVTI |
| | | SKDNSKNQVSLKLSSVTAADTAVYYCAKHYYYGGSYAMDYWG |
| | | QGTLVTVSSGGGGSGGGGSGGGGSEIVMTQSPATLSLSPGERATL |
| | | SCRASQDISKYLNWYQQKPGQAPRLLIYHTSRLHSGIPARFSGSG |
| | | SGTDYTLTISSLQPEDFAVYFCQQGNTLPYTFGQGTKLEIKTTTPA |
| | | PRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDG |
| | | CSCRFPEEEEGGCELRVKFSRSADAPAYKQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |

Humanized CAR5

| | | |
|---|---|---|
| SEQ ID | CAR5 scFv | EIVMTQSPATLSLSPGERATLSCRASQDISKYLNWYQQKPGQAPR |
| NO: 1078 | domain | LLIYHTSRLHSGIPARFSGSGSGTDYTLTISSLQPEDFAVYFCQQG |
| | | NTLPYTFGQGTKLEIKGGGGSGGGGSGGGGSGGGGSQVQLQESG |
| | | PGLVKPSETLSLTCTVSGVSLPDYGVSWIRQPPGKGLEWIGVIWG |
| | | SETTYYSSSLKSRVTISKDNSKNQVSLKLSSVTAADTAVYYCAKH |
| | | YYYGGSYAMDYWGQGTLVTVSS |
| SEQ ID | CAR 5-Full- | MALPVTALLLPLALLLHAARPEIVMTQSPATLSLSPGERATLSCR |
| NO: 1079 | aa | ASQDISKYLNWYQQKPGQAPRLLIYHTSRLHSGIPARFSGSGSGT |
| | | DYTLTISSLQPEDFAVYFCQQGNTLPYTFGQGTKLEIKGGGGSGG |
| | | GGSGGGGSGGGGSQVQLQESGPGLVKPSETLSLTCTVSGVSLPD |
| | | YGVSWIRQPPGKGLEWIGVIWGSETTYYSSSLKSRVTISKDNSKN |
| | | QVSLKLSSVTAADTAVYYCAKHYYYGGSYAMDYWGQGTLVTV |
| | | SSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |

Humanized CAR6

| | | |
|---|---|---|
| SEQ ID | CAR6 | EIVMTQSPATLSLSPGERATLSCRASQDISKYLNWYQQKPGQAPR |
| NO: 1080 | scFv domain | LLIYHTSRLHSGIPARFSGSGSGTDYTLTISSLQPEDFAVYFCQQG |
| | | NTLPYTFGQGTKLEIKGGGGSGGGGSGGGGSGGGGSQVQLQESG |
| | | PGLVKPSETLSLTCTVSGVSLPDYGVSWIRQPPGKGLEWIGVIWG |
| | | SETTYYQSSLKSRVTISKDNSKNQVSLKLSSVTAADTAVYYCAK |
| | | HYYYGGSYAMDYWGQGTLVTVSS |
| SEQ ID | CAR6- | MALPVTALLLPLALLLHAARPEIVMTQSPATLSLSPGERATLSCR |
| NO: 1081 | Full-aa | ASQDISKYLNWYQQKPGQAPRLLIYHTSRLHSGIPARFSGSGSGT |
| | | DYTLTISSLQPEDFAVYFCQQGNTLPYTFGQGTKLEIKGGGGSGG |
| | | GGSGGGGSGGGGSQVQLQESGPGLVKPSETLSLTCTVSGVSLPD |
| | | YGVSWIRQPPGKGLEWIGVIWGSETTYYQSSLKSRVTISKDNSKN |
| | | QVSLKLSSVTAADTAVYYCAKHYYYGGSYAMDYWGQGTLVTV |
| | | SSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |

Humanized CAR7

| | | |
|---|---|---|
| SEQ ID | CAR7 scFv | QVQLQESGPGLVKPSETLSLTCTVSGVSLPDYGVSWIRQPPGKGL |
| NO: 1082 | domain | EWIGVIWGSETTYYSSSLKSRVTISKDNSKNQVSLKLSSVTAADT |
| | | AVYYCAKHYYYGGSYAMDYWGQGTLVTVSSGGGGSGGGGSG |
| | | GGGSGGGGSEIVMTQSPATLSLSPGERATLSCRASQDISKYLNWY |
| | | QQKPGQAPRLLIYHTSRLHSGIPARFSGSGSGTDYTLTISSLQPEDF |
| | | AVYFCQQGNTLPYTFGQGTKLEIK |
| SEQ ID | CAR 7 Full- | MALPVTALLLPLALLLHAARPQVQLQESGPGLVKPSETLSLTCTV |
| NO: 1083 | aa | SGVSLPDYGVSWIRQPPGKGLEWIGVIWGSETTYYSSSLKSRVTIS |
| | | KDNSKNQVSLKLSSVTAADTAVYYCAKHYYYGGSYAMDYWGQ |
| | | GTLVTVSSGGGGSGGGGSGGGGSGGGGSEIVMTQSPATLSLSPG |
| | | ERATLSCRASQDISKYLNWYQQKPGQAPRLLIYHTSRLHSGIPAR |
| | | FSGSGSGTDYTLTISSLQPEDFAVYFCQQGNTLPYTFGQGTKLEIK |
| | | TTTPAPRPPTPAPTIASQPLSLRPEACRPAAGGAVHTRGLDFACDI |
| | | YIWAPLAGTCGVLLLSLVITLYCKRGRKKLLYIFKQPFMRPVQTT |
| | | QEEDGCSCRFPEEEEGGCELRVKFSRSADAPAYKQGQNQLYNEL |
| | | NLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |

Humanized CAR8

| | | |
|---|---|---|
| SEQ ID | CAR8 scFv | QVQLQESGPGLVKPSETLSLTCTVSGVSLPDYGVSWIRQPPGKGL |
| NO: 1084 | domain | EWIGVIWGSETTYYQSSLKSRVTISKDNSKNQVSLKLSSVTAADT |
| | | AVYYCAKHYYYGGSYAMDYWGQGTLVTVSSGGGGSGGGGSG |
| | | GGGSGGGGSEIVMTQSPATLSLSPGERATLSCRASQDISKYLNWY |
| | | QQKPGQAPRLLIYHTSRLHSGIPARFSGSGSGTDYTLTISSLQPEDF |
| | | AVYFCQQGNTLPYTFGQGTKLEIK |
| SEQ ID | CAR 8-Full- | MALPVTALLLPLALLLHAARPQVQLQESGPGLVKPSETLSLTCTV |
| NO: 1085 | aa | SGVSLPDYGVSWIRQPPGKGLEWIGVIWGSETTYYQSSLKSRVTI |
| | | SKDNSKNQVSLKLSSVTAADTAVYYCAKHYYYGGSYAMDYWG |
| | | QGTLVTVSSGGGGSGGGGSGGGGSGGGGSEIVMTQSPATLSLSP |
| | | GERATLSCRASQDISKYLNWYQQKPGQAPRLLIYHTSRLHSGIPA |
| | | RFSGSGSGTDYTLTISSLQPEDFAVYFCQQGNTLPYTFGQGTKLEI |
| | | KTTTPAPRPPTPAPTIASQPLSLRPEACRPAAGGAVHTRGLDFACD |
| | | IYIWAPLAGTCGVLLLSLVITLYCKRGRKKLLYIFKQPFMRPVQT |
| | | TQEEDGCSCRFPEEEEGGCELRVKFSRSADAPAYKQGQNQLYNE |
| | | LNLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |

Humanized CAR9

| | | |
|---|---|---|
| SEQ ID | CAR9 scFv | EIVMTQSPATLSLSPGERATLSCRASQDISKYLNWYQQKPGQAPR |
| NO: 1086 | domain | LLIYHTSRLHSGIPARFSGSGSGTDYTLTISSLQPEDFAVYFCQQG |
| | | NTLPYTFGQGTKLEIKGGGGSGGGGSGGGGSGGGGSQVQLQESG |
| | | PGLVKPSETLSLTCTVSGVSLPDYGVSWIRQPPGKGLEWIGVIWG |
| | | SETTYYNSSLKSRVTISKDNSKNQVSLKLSSVTAADTAVYYCAK |
| | | HYYYGGSYAMDYWGQGTLVTVSS |
| SEQ ID | CAR 9-Full- | MALPVTALLLPLALLLHAARPEIVMTQSPATLSLSPGERATLSCR |
| NO: 1087 | aa | ASQDISKYLNWYQQKPGQAPRLLIYHTSRLHSGIPARFSGSGSGT |
| | | DYTLTISSLQPEDFAVYFCQQGNTLPYTFGQGTKLEIKGGGGSGG |
| | | GGSGGGGSGGGGSQVQLQESGPGLVKPSETLSLTCTVSGVSLPD |
| | | YGVSWIRQPPGKGLEWIGVIWGSETTYYNSSLKSRVTISKDNSKN |
| | | QVSLKLSSVTAADTAVYYCAKHYYYGGSYAMDYWGQGTLVTV |
| | | SSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |

Humanized CAR10

| | | |
|---|---|---|
| SEQ ID | CAR10 scFv | QVQLQESGPGLVKPSETLSLTCTVSGVSLPDYGVSWIRQPPGKGL |
| NO: 1088 | domain | EWIGVIWGSETTYYNSSLKSRVTISKDNSKNQVSLKLSSVTAADT |
| | | AVYYCAKHYYYGGSYAMDYWGQGTLVTVSSGGGGSGGGGSG |
| | | GGGSGGGGSEIVMTQSPATLSLSPGERATLSCRASQDISKYLNWY |
| | | QQKPGQAPRLLIYHTSRLHSGIPARFSGSGSGTDYTLTISSLQPEDF |
| | | AVYFCQQGNTLPYTFGQGTKLEIK |
| SEQ ID | CAR 10 Full- | MALPVTALLLPLALLLHAARPEIVMTQSPATLSLSPGERATLSCR |
| NO: 1089 | aa | ASQDISKYLNWYQQKPGQAPRLLIYHTSRLHSGIPARFSGSGSGT |
| | | DYTLTISSLQPEDFAVYFCQQGNTLPYTFGQGTKLEIKGGGGSGG |
| | | GGSGGGGSGGGGSQVQLQESGPGLVKPSETLSLTCTVSGVSLPD |
| | | YGVSWIRQPPGKGLEWIGVIWGSETTYYNSSLKSRVTISKDNSKN |
| | | QVSLKLSSVTAADTAVYYCAKHYYYGGSYAMDYWGQGTLVTV |
| | | SSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |

Humanized CAR11

| | | |
|---|---|---|
| SEQ ID | CAR11 scFv | EIVMTQSPATLSLSPGERATLSCRASQDISKYLNWYQQKPGQAPR |
| NO: 1090 | domain | LLIYHTSRLHSGIPARFSGSGSGTDYTLTISSLQPEDFAVYFCQQG |
| | | NTLPYTFGQGTKLEIKGGGGSGGGGSGGGGSQVQLQESGPGLVK |
| | | PSETLSLTCTVSGVSLPDYGVSWIRQPPGKGLEWIGVIWGSETTY |
| | | YNSSLKSRVTISKDNSKNQVSLKLSSVTAADTAVYYCAKHYYYG |
| | | GSYAMDYWGQGTLVTVSS |
| SEQ ID | CAR 11 Full- | MALPVTALLLPLALLLHAARPQVQLQESGPGLVKPSETLSLTCTV |
| NO: 1091 | aa | SGVSLPDYGVSWIRQPPGKGLEWIGVIWGSETTYYNSSLKSRVTI |
| | | SKDNSKNQVSLKLSSVTAADTAVYYCAKHYYYGGSYAMDYWG |
| | | QGTLVTVSSGGGGSGGGGSGGGGSGGGGSEIVMTQSPATLSLSP |
| | | GERATLSCRASQDISKYLNWYQQKPGQAPRLLIYHTSRLHSGIPA |
| | | RFSGSGSGTDYTLTISSLQPEDFAVYFCQQGNTLPYTFGQGTKLEI |
| | | KTTTPAPRPPTPAPTIASQPLSLRPEACRPAAGGAVHTRGLDFACD |
| | | IYIWAPLAGTCGVLLLSLVITLYCKRGRKKLLYIFKQPFMRPVQT |
| | | TQEEDGCSCRFPEEEEGGCELRVKFSRSADAPAYKQGQNQLYNE |
| | | LNLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |

Humanized CAR12

| | | |
|---|---|---|
| SEQ ID | CAR12 | QVQLQESGPGLVKPSETLSLTCTVSGVSLPDYGVSWIRQPPGKGL |
| NO: 1092 | scFv domain | EWIGVIWGSETTYYNSSLKSRVTISKDNSKNQVSLKLSSVTAADT |
| | | AVYYCAKHYYYGGSYAMDYWGQGTLVTVSSGGGGSGGGGSG |
| | | GGGSEIVMTQSPATLSLSPGERATLSCRASQDISKYLNWYQQKPG |
| | | QAPRLLIYHTSRLHSGIPARFSGSGSGTDYTLTISSLQPEDFAVYFC |
| | | QQGNTLPYTFGQGTKLEIK |
| SEQ ID | CAR 12- | MALPVTALLLPLALLLHAARPEIVMTQSPATLSLSPGERATLSCR |
| NO: 1093 | Full-aa | ASQDISKYLNWYQQKPGQAPRLLIYHTSRLHSGIPARFSGSGSGT |
| | | DYTLTISSLQPEDFAVYFCQQGNTLPYTFGQGTKLEIKGGGGSGG |
| | | GGSGGGGSQVQLQESGPGLVKPSETLSLTCTVSGVSLPDYGVSWI |
| | | RQPPGKGLEWIGVIWGSETTYYNSSLKSRVTISKDNSKNQVSLKL |
| | | SSVTAADTAVYYCAKHYYYGGSYAMDYWGQGTLVTVSSTTTP |
| | | APRPPTPAPTIASQPLSLRPEACRPAAGGAVHTRGLDFACDIYIWA |
| | | PLAGTCGVLLLSLVITLYCKRGRKKLLYIFKQPFMRPVQTTQEED |
| | | GCSCRFPEEEEGGCELRVKFSRSADAPAYKQGQNQLYNELNLGR |
| | | REEYDVLDKRRGRDPEMGGKPRRKNPQEGLYNELQKDKMAEA |
| | | YSEIGMKGERRRGKGHDGLYQGLSTATKDTYDALHMQALPPR |

CAR A

| | | |
|---|---|---|
| SEQ ID | CAR A- | atgcttctcctggtgacaagccttctgctctgtgagttaccacacccagcatt |
| NO: 3215 | with leader | cctcctgatcccagacatccagatgacacagactacatcctccctgtctgcct |
| | Full-nt; | ctctgggagacagagtcaccatcagttgcagggcaagtcaggacattagtaaa |
| | | tatttaaattggtatcagcagaaaccagatggaactgttaaactcctgatcta |
| | | ccatacatcaagattacactcaggagtcccatcaaggttcagtggcagtgggt |
| | | ctggaacagattattctctcaccattagcaacctggagcaagaagatattgcc |
| | | acttacttttgccaacagggtaatacgcttccgtacacgttcggaggggggac |
| | | taagttggaaataacaggctccacctctggatccggcaagcccggatctggcg |
| | | agggatccaccaagggcgaggtgaaactgcaggagtcaggacctggcctggtg |
| | | gcgccctcacagagcctgtccgtcacatgcactgtctcaggggtctcattacc |
| | | cgactatggtgtaagctggattcgccagcctccacgaaagggtctggagtggc |
| | | tgggagtaatatggggtagtgaaaccacatactataattcagctctcaaatcc |
| | | agactgaccatcatcaaggacaactccaagagccaagttttcttaaaaatgaa |
| | | cagtctgcaaactgatgacacagccatttactactgtgccaaacattattact |
| | | acggtggtagctatgctatggactactggggtcaaggaacctcagtcaccgtc |
| | | tcctcagcggccgcaattgaagttatgtatcctcctccttacctagacaatga |
| | | gaagagcaatggaaccattatccatgtgaaagggaaacacctttgtccaagtc |
| | | ccctatttcccggaccttctaagcccttttgggtgctggtggtggttggggga |
| | | gtcctggcttgctatagcttgctagtaacagtggcctttattattttctgggt |
| | | gaggagtaagaggagcaggctcctgcacagtgactacatgaacatgactcccc |
| | | gccgccccgggcccacccgcaagcattaccagccctatgccccaccacgcgac |
| | | ttcgcagcctatcgctccagagtgaagttcagcaggagcgcagacgcccccgc |
| | | gtaccagcagggccagaaccagctctataacgagctcaatctaggacgaagag |
| | | aggagtacgatgttttggacaagagacgtggccgggaccctgagatgggggga |
| | | aagccgagaaggaagaaccctcaggaaggcctgtacaatgaactgcagaaaga |
| | | taagatggcggaggcctacagtgagattgggatgaaaggcgagcgccggaggg |
| | | gcaaggggcacgatggcctttaccagggtctcagtacagccaccaaggacacc |
| | | tacgacgcccttcacatgcaggccctgccccctcgc |
| SEQ ID | CAR A-full | MLLLVTSLLLCELPHPAFLLIPDIQMTQTTSSLSASLGDRVTISCRASQDISK |
| NO: 3216 | amino acid | YLNWYQQKPDGTVKLLIYHTSRLHSGVPSRFSGSGSGTDYSLTISNLEQEDIA |
| | transgene | TYFCQQGNTLPYTFGGGTKLEITGSTSGSGKPGSGEGSTKGEVKLQESGPGLV |
| | sequence; | APSQSLSVTCTVSGVSLPDYGVSWIRQPPRKGLEWLGVIWGSETTYYNSALKS |
| | with leader | RLTIIKDNSKSQVFLKMNSLQTDDTAIYYCAKHYYYGGSYAMDYWGQGTSVTV |
| | underlined | SSAAAIEVMYPPPYLDNEKSNGTIIHVKGKHLCPSPLFPGPSKPFWVLVVVGG |
| | | VLACYSLLVTVAFIIFWVRSKRSRLLHSDYMNMTPRRPGPTRKHYQPYAPPRD |
| | | FAAYRSRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDT |
| | | YDALHMQALPPR |
| SEQ ID | CAR A- | atgcttctcctggtgacaagccttctgctctgtgagttaccacacccagcatt |
| NO: 3217 | CD19 scFv | cctcctgatcccagacatccagatgacacagactacatcctccctgtctgcct |
| | nucleotide | ctctgggagacagagtcaccatcagttgcagggcaagtcaggacattagtaaa |
| | sequence | tatttaaattggtatcagcagaaaccagatggaactgttaaactcctgatcta |
| | with leader | ccatacatcaagattacactcaggagtcccatcaaggttcagtggcagtgggt |
| | underlined | ctggaacagattattctctcaccattagcaacctggagcaagaagatattgcc |
| | | acttacttttgccaacagggtaatacgcttccgtacacgttcggagggggga |
| | | taagttggaaataacaggctccacctctggatccggcaagcccggatctggcg |
| | | agggatccaccaagggcgaggtgaaactgcaggagtcaggacctggcctggtg |
| | | gcgccctcacagagcctgtccgtcacatgcactgtctcaggggtctcattacc |
| | | cgactatggtgtaagctggattcgccagcctccacgaaagggtctggagtgg |
| | | tgggagtaatatggggtagtgaaaccacatactataattcagctctcaaatcc |
| | | agactgaccatcatcaaggacaactccaagagccaagttttcttaaaaatgaa |
| | | cagtctgcaaactgatgacacagccatttactactgtgccaaacattattact |
| | | acggtggtagctatgctatggactactggggtcaaggaacctcagtcaccgtc |
| | | tcctca |
| SEQ ID | CAR A- | MLLLVTSLLLCELPHPAFLLIPDIQMTQTTSSLSASLGDRVTISCRASQDISK |
| NO: 3218 | CD19 scFv | YLNWYQQKPDGTVKLLIYHTSRLHSGVPSRFSGSGSGTDYSLTISNLEQEDIA |
| | amino acid | TYFCQQGNTLPYTFGGGTKLEITGSTSGSGKPGSGEGSTKGEVKLQESGPGLV |
| | sequence; | APSQSLSVTCTVSGVSLPDYGVSWIRQPPRKGLEWLGVIWGSETTYYNSALKS |
| | with leader | RLTIIKDNSKSQVFLKMNSLQTDDTAIYYCAKHYYYGGSYAMDYWGQGTSVTV |
| | underlined | S |
| SEQ ID | CAR A-full | gacatccagatgacacagactacatcctccctgtctgcctctctgggagacag |
| NO: 3219 | nucleotide | agtcaccatcagttgcagggcaagtcaggacattagtaaatatttaaattggt |
| | sequence; no | atcagcagaaaccagatggaactgttaaactcctgatctaccatacatcaaga |
| | leader | ttacactcaggagtcccatcaaggttcagtggcagtgggtctggaacagatta |
| | | ttctctcaccattagcaacctggagcaagaagatattgccacttacttttgcc |
| | | aacagggtaatacgcttccgtacacgttcggaggggggactaagttggaaata |
| | | acaggctccacctctggatccggcaagcccggatctggcgagggatccaccaa |
| | | gggcgaggtgaaactgcaggagtcaggacctggcctggtggcgccctcacaga |
| | | gcctgtccgtcacatgcactgtctcaggggtctcattacccgactatggtgta |
| | | agctggattcgccagcctccacgaaagggtctggagtggctgggagtaatatg |
| | | gggtagtgaaaccacatactataattcagctctcaaatccagactgaccatca |
| | | tcaaggacaactccaagagccaagttttcttaaaaatgaacagtctgcaaact |
| | | gatgacacagccatttactactgtgccaaacattattactacggtggtagcta |
| | | tgctatggactactggggtcaaggaacctcagtcaccgtctcctcagcggccg |
| | | caattgaagttatgtatcctcctccttacctagacaatgagaagagcaatgga |
| | | accattatccatgtgaaagggaaacacctttgtccaagtcccctatttcccgg |
| | | accttctaagcccttttgggtgctggtggtggttgggggagtcctggcttgct |
| | | atagcttgctagtaacagtggcctttattattttctgggtgaggagtaagagg |
| | | agcaggctcctgcacagtgactacatgaacatgactccccgccgccccgggcc |
| | | cacccgcaagcattaccagccctatgccccaccacgcgacttcgcagcctatc |
| | | gctccagagtgaagttcagcaggagcgcagacgcccccgcgtaccagcagggc |
| | | cagaaccagctctataacgagctcaatctaggacgaagagaggagtacgatgt |
| | | tttggacaagagacgtggccgggaccctgagatggggggaaagccgagaagga |
| | | agaaccctcaggaaggcctgtacaatgaactgcagaaagataagatggcggag |
| | | gcctacagtgagattgggatgaaaggcgagcgccggaggggcaaggggcacga |
| | | tggcctttaccagggtctcagtacagccaccaaggacacctacgacgcccttc |
| | | acatgcaggccctgccccctcgc |
| SEQ ID | CAR A-full | DIQMTQTTSSLSASLGDRVTISCRASQDISKYLNWYQQKPDGTVKLLIYHTSR |
| NO: 1064 | amino acid | LHSGVPSRFSGSGSGTDYSLTISNLEQEDIATYFCQQGNTLPYTFGGGTKLEI |
| | transgene | TGSTSGSGKPGSGEGSTKGEVKLQESGPGLVAPSQSLSVTCTVSGVSLPDYGV |
| | sequence; no | SWIRQPPRKGLEWLGVIWGSETTYYNSALKSRLTIIKDNSKSQVFLKMNSLQT |
| | leader | DDTAIYYCAKHYYYGGSYAMDYWGQGTSVTVSSAAAIEVMYPPPYLDNEKSNG |
| | | TIIHVKGKHLCPSPLFPGPSKPFWVLVVVGGVLACYSLLVTVAFIIFWVRSKR |
| | | SRLLHSDYMNMTPRRPGPTRKHYQPYAPPRDFAAYRSRVKFSRSADAPAYQQG |
| | | QNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | CAR A- | gacatccagatgacacagactacatcctccctgtctgcctctctgggagacag |
| NO: 3220 | CD19 scFv | agtcaccatcagttgcagggcaagtcaggacattagtaaatatttaaattggt |
| | nucleotide; | atcagcagaaaccagatggaactgttaaactcctgatctaccatacatcaaga |
| | no leader | ttacactcaggagtcccatcaaggttcagtggcagtgggtctggaacagatta |
| | | ttctctcaccattagcaacctggagcaagaagatattgccacttacttttgcc |
| | | aacagggtaatacgcttccgtacacgttcggaggggggactaagttggaaata |
| | | acaggctccacctctggatccggcaagcccggatctggcgagggatccaccaa |
| | | gggcgaggtgaaactgcaggagtcaggacctggcctggtggcgccctcacaga |
| | | gcctgtccgtcacatgcactgtctcaggggtctcattacccgactatggtgta |
| | | agctggattcgccagcctccacgaaagggtctggagtggctgggagtaatatg |
| | | gggtagtgaaaccacatactataattcagctctcaaatccagactgaccatca |
| | | tcaaggacaactccaagagccaagttttcttaaaaatgaacagtctgcaaact |
| | | gatgacacagccatttactactgtgccaaacattattactacggtggtagcta |
| | | tgctatggactactggggtcaaggaacctcagtcaccgtctcctca |
| SEQ ID | CAR A- | DIQMTQTTSSLSASLGDRVTISCRASQDISKYLNWYQQKPDGTVKLLIYHTSR |
| NO: 1063 | CD19 scFv | LHSGVPSRFSGSGSGTDYSLTISNLEQEDIATYFCQQGNTLPYTFGGGTKLEI |
| | amino acid | TGSTSGSGKPGSGEGSTKGEVKLQESGPGLVAPSQSLSVTCTVSGVSLPDYGV |
| | sequence; no | SWIRQPPRKGLEWLGVIWGSETTYYNSALKSRLTIIKDNSKSQVFLKMNSLQT |
| | leader | DDTAIYYCAKHYYYGGSYAMDYWGQGTSVTVSS |

CAR B

| | | |
|---|---|---|
| SEQ ID | CAR B-full | ATGCTGCTGCTGGTGACCAGCCTGCTGCTGTGCGAGCTGCCCCACCCCGCCTT |
| NO: 3221 | sequence; | TCTGCTGATCCCCGACATCCAGATGACCCAGACCACCTCCAGCCTGAGCGCCA |
| | with leader | GCCTGGGCGACCGGGTGACCATCAGCTGCCGGGCCAGCCAGGACATCAGCAAG |
| | underlined | TACCTGAACTGGTATCAGCAGAAGCCCGACGGCACCGTCAAGCTGCTGATCTA |
| | nucleotide | CCACACCAGCCGGCTGCACAGCGGCGTGCCCAGCCGGTTTAGCGGCAGCGGCT |
| | | CCGGCACCGACTACAGCCTGACCATCTCCAACCTGGAACAGGAAGATATCGCC |
| | | ACCTACTTTTGCCAGCAGGGCAACACACTGCCCTACACCTTTGGCGGCGGAAC |
| | | AAAGCTGGAAATCACCGGCAGCACCTCCGGCAGCGGCAAGCCTGGCAGCGGCG |
| | | AGGGCAGCACCAAGGGCGAGGTGAAGCTGCAGGAAAGCGGCCCTGGCCTGGTG |
| | | GCCCCCAGCCAGAGCCTGAGCGTGACCTGCACCGTGAGCGGCGTGAGCCTGCC |
| | | CGACTACGGCGTGAGCTGGATCCGGCAGCCCCCCAGGAAGGGCCTGGAATGGC |
| | | TGGGCGTGATCTGGGGCAGCGAGACCACCTACTACAACAGCGCCCTGAAGAGC |
| | | CGGCTGACCATCATCAAGGACAACAGCAAGAGCCAGGTGTTCCTGAAGATGAA |
| | | CAGCCTGCAGACCGACGACACCGCCATCTACTACTGCGCCAAGCACTACTACT |
| | | ACGGCGGCAGCTACGCCATGGACTACTGGGGCCAGGGCACCAGCGTGACCGTG |
| | | AGCAGCGAATCTAAGTACGGACCGCCCTGCCCCCCTTGCCCTATGTTCTGGGT |
| | | GCTGGTGGTGGTCGGAGGCGTGCTGGCCTGCTACAGCCTGCTGGTCACCGTGG |
| | | CCTTCATCATCTTTTGGGTGAAACGGGGCAGAAAGAAACTCCTGTATATATTC |
| | | AAACAACCATTTATGAGACCAGTACAAACTACTCAAGAGGAAGATGGCTGTAG |
| | | CTGCCGATTTCCAGAAGAAGAAGAAGGAGGATGTGAACTGCGGGTGAAGTTCA |
| | | GCAGAAGCGCCGACGCCCCTGCCTACCAGCAGGGCCAGAATCAGCTGTACAAC |
| | | GAGCTGAACCTGGGCAGAAGGGAAGAGTACGACGTCCTGGATAAGCGGAGAGG |
| | | CCGGGACCCTGAGATGGGCGGCAAGCCTCGGCGGAAGAACCCCCAGGAAGGCC |
| | | TGTATAACGAACTGCAGAAAGACAAGATGGCCGAGGCCTACAGCGAGATCGGC |
| | | ATGAAGGGCGAGCGGAGGCGGGGCAAGGGCCACGACGGCCTGTATCAGGGCCT |
| | | GTCCACCGCCACCAAGGATACCTACGACGCCCTGCACATGCAGGCCCTGCCCC |
| | | CAAGG |
| SEQ ID | CAR B-full | MLLLVTSLLLCELPHPAFLLIPDIQMTQTTSSLSASLGDRVTISCRASQDISK |
| NO: 3222 | transgene | YLNWYQQKPDGTVKLLIYHTSRLHSGVPSRFSGSGSGTDYSLTISNLEQEDIA |
| | amino acid | TYFCQQGNTLPYTFGGGTKLEITGSTSGSGKPGSGEGSTKGEVKLQESGPGLV |
| | sequence; | APSQSLSVTCTVSGVSLPDYGVSWIRQPPRKGLEWLGVIWGSETTYYNSALKS |
| | with leader | RLTIIKDNSKSQVFLKMNSLQTDDTAIYYCAKHYYYGGSYAMDYWGQGTSVTV |
| | underlined | SSESKYGPPCPPCPMFWVLVVVGGVLACYSLLVTVAFIIFWVKRGRKKLLYIF |
| | | KQPFMRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| SEQ ID | CAR B- | ATGCTGCTGCTGGTGACCAGCCTGCTGCTGTGCGAGCTGCCCCACCCCGCCTT |
| NO: 3223 | sequence; | TCTGCTGATCCCCGACATCCAGATGACCCAGACCACCTCCAGCCTGAGCGCCA |
| | with leader | GCCTGGGCGACCGGGTGACCATCAGCTGCCGGGCCAGCCAGGACATCAGCAAG |
| | underlined | TACCTGAACTGGTATCAGCAGAAGCCCGACGGCACCGTCAAGCTGCTGATCTA |
| | CD19 scFv | CCACACCAGCCGGCTGCACAGCGGCGTGCCCAGCCGGTTTAGCGGCAGCGGCT |
| | nucleotide | CCGGCACCGACTACAGCCTGACCATCTCCAACCTGGAACAGGAAGATATCGCC |
| | | ACCTACTTTTGCCAGCAGGGCAACACACTGCCCTACACCTTTGGCGGCGGAAC |
| | | AAAGCTGGAAATCACCGGCAGCACCTCCGGCAGCGGCAAGCCTGGCAGCGGCG |
| | | AGGGCAGCACCAAGGGCGAGGTGAAGCTGCAGGAAAGCGGCCCTGGCCTGGTG |
| | | GCCCCCAGCCAGAGCCTGAGCGTGACCTGCACCGTGAGCGGCGTGAGCCTGCC |
| | | CGACTACGGCGTGAGCTGGATCCGGCAGCCCCCCAGGAAGGGCCTGGAATGGC |
| | | TGGGCGTGATCTGGGGCAGCGAGACCACCTACTACAACAGCGCCCTGAAGAGC |
| | | CGGCTGACCATCATCAAGGACAACAGCAAGAGCCAGGTGTTCCTGAAGATGAA |
| | | CAGCCTGCAGACCGACGACACCGCCATCTACTACTGCGCCAAGCACTACTACT |
| | | ACGGCGGCAGCTACGCCATGGACTACTGGGGCCAGGGCACCAGCGTGACCGTG |
| | | AGCAGC |
| SEQ ID | CAR B- | MLLLVTSLLLCELPHPAFLLIPDIQMTQTTSSLSASLGDRVTISCRASQDISK |
| NO: 3224 | amino acid | YLNWYQQKPDGTVKLLIYHTSRLHSGVPSRFSGSGSGTDYSLTISNLEQEDIA |
| | sequence; | TYFCQQGNTLPYTFGGGTKLEITGSTSGSGKPGSGEGSTKGEVKLQESGPGLV |
| | with leader | APSQSLSVTCTVSGVSLPDYGVSWIRQPPRKGLEWLGVIWGSETTYYNSALKS |
| | underlined | RLTIIKDNSKSQVFLKMNSLQTDDTAIYYCAKHYYYGGSYAMDYWGQGTSVTV |
| | CD19 scFv | SS |
| SEQ ID | CAR B-full | GACATCCAGATGACCCAGACCACCTCCAGCCTGAGCGCCAGCCTGGGCGACCG |
| NO: 3225 | nucleotide | GGTGACCATCAGCTGCCGGGCCAGCCAGGACATCAGCAAGTACCTGAACTGGT |
| | sequence; no | ATCAGCAGAAGCCCGACGGCACCGTCAAGCTGCTGATCTACCACACCAGCCGG |
| | leader | CTGCACAGCGGCGTGCCCAGCCGGTTTAGCGGCAGCGGCTCCGGCACCGACTA |
| | | CAGCCTGACCATCTCCAACCTGGAACAGGAAGATATCGCCACCTACTTTTGCC |
| | | AGCAGGGCAACACACTGCCCTACACCTTTGGCGGCGGAACAAAGCTGGAAATC |
| | | ACCGGCAGCACCTCCGGCAGCGGCAAGCCTGGCAGCGGCGAGGGCAGCACCAA |
| | | GGGCGAGGTGAAGCTGCAGGAAAGCGGCCCTGGCCTGGTGGCCCCCAGCCAGA |
| | | GCCTGAGCGTGACCTGCACCGTGAGCGGCGTGAGCCTGCCCGACTACGGCGTG |
| | | AGCTGGATCCGGCAGCCCCCCAGGAAGGGCCTGGAATGGCTGGGCGTGATCTG |
| | | GGGCAGCGAGACCACCTACTACAACAGCGCCCTGAAGAGCCGGCTGACCATCA |
| | | TCAAGGACAACAGCAAGAGCCAGGTGTTCCTGAAGATGAACAGCCTGCAGACC |
| | | GACGACACCGCCATCTACTACTGCGCCAAGCACTACTACTACGGCGGCAGCTA |
| | | CGCCATGGACTACTGGGGCCAGGGCACCAGCGTGACCGTGAGCAGCGAATCTA |
| | | AGTACGGACCGCCCTGCCCCCCTTGCCCTATGTTCTGGGTGCTGGTGGTGGTC |
| | | GGAGGCGTGCTGGCCTGCTACAGCCTGCTGGTCACCGTGGCCTTCATCATCTT |
| | | TTGGGTGAAACGGGGCAGAAAGAAACTCCTGTATATATTCAAACAACCATTTA |
| | | TGAGACCAGTACAAACTACTCAAGAGGAAGATGGCTGTAGCTGCCGATTTCCA |
| | | GAAGAAGAAGAAGGAGGATGTGAACTGCGGGTGAAGTTCAGCAGAAGCGCCGA |
| | | CGCCCCTGCCTACCAGCAGGGCCAGAATCAGCTGTACAACGAGCTGAACCTGG |
| | | GCAGAAGGGAAGAGTACGACGTCCTGGATAAGCGGAGAGGCCGGGACCCTGAG |
| | | ATGGGCGGCAAGCCTCGGCGGAAGAACCCCCAGGAAGGCCTGTATAACGAACT |
| | | GCAGAAAGACAAGATGGCCGAGGCCTACAGCGAGATCGGCATGAAGGGCGAGC |
| | | GGAGGCGGGGCAAGGGCCACGACGGCCTGTATCAGGGCCTGTCCACCGCCACC |
| | | AAGGATACCTACGACGCCCTGCACATGCAGGCCCTGCCCCCAAGG |
| SEQ ID | CAR B-full | DIQMTQTTSSLSASLGDRVTISCRASQDISKYLNWYQQKPDGTVKLLIYHTSR |
| NO: 3226 | amino acid | LHSGVPSRFSGSGSGTDYSLTISNLEQEDIATYFCQQGNTLPYTFGGGTKLEI |
| | transgene | TGSTSGSGKPGSGEGSTKGEVKLQESGPGLVAPSQSLSVTCTVSGVSLPDYGV |
| | sequence; no | SWIRQPPRKGLEWLGVIWGSETTYYNSALKSRLTIIKDNSKSQVFLKMNSLQT |
| | leader | DDTAIYYCAKHYYYGGSYAMDYWGQGTSVTVSSESKYGPPCPPCPMFWVLVVV |
| | | GGVLACYSLLVTVAFIIFWVKRGRKKLLYIFKQPFMRPVQTTQEEDGCSCRFP |
| | | EEEEGGCELRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGLSTAT |
| | | KDTYDALHMQALPPR |
| SEQ ID | CAR B- | GACATCCAGATGACCCAGACCACCTCCAGCCTGAGCGCCAGCCTGGGCGACCG |
| NO: 3227 | CD19 scFv | GGTGACCATCAGCTGCCGGGCCAGCCAGGACATCAGCAAGTACCTGAACTGGT |
| | sequence; no | ATCAGCAGAAGCCCGACGGCACCGTCAAGCTGCTGATCTACCACACCAGCCGG |
| | leader | CTGCACAGCGGCGTGCCCAGCCGGTTTAGCGGCAGCGGCTCCGGCACCGACTA |
| | | CAGCCTGACCATCTCCAACCTGGAACAGGAAGATATCGCCACCTACTTTTGCC |
| | | AGCAGGGCAACACACTGCCCTACACCTTTGGCGGCGGAACAAAGCTGGAAATC |
| | | ACCGGCAGCACCTCCGGCAGCGGCAAGCCTGGCAGCGGCGAGGGCAGCACCAA |
| | | GGGCGAGGTGAAGCTGCAGGAAAGCGGCCCTGGCCTGGTGGCCCCCAGCCAGA |
| | | GCCTGAGCGTGACCTGCACCGTGAGCGGCGTGAGCCTGCCCGACTACGGCGTG |
| | | AGCTGGATCCGGCAGCCCCCCAGGAAGGGCCTGGAATGGCTGGGCGTGATCTG |
| | | GGGCAGCGAGACCACCTACTACAACAGCGCCCTGAAGAGCCGGCTGACCATCA |
| | | TCAAGGACAACAGCAAGAGCCAGGTGTTCCTGAAGATGAACAGCCTGCAGACC |
| | | GACGACACCGCCATCTACTACTGCGCCAAGCACTACTACTACGGCGGCAGCTA |
| | | CGCCATGGACTACTGGGGCCAGGGCACCAGCGTGACCGTGAGCAGC |
| SEQ ID | CAR B- | DIQMTQTTSSLSASLGDRVTISCRASQDISKYLNWYQQKPDGTVKLLIYHTSR |
| NO: 1063 | CD19 scFv | LHSGVPSRFSGSGSGTDYSLTISNLEQEDIATYFCQQGNTLPYTFGGGTKLEI |
| | sequence; no | TGSTSGSGKPGSGEGSTKGEVKLQESGPGLVAPSQSLSVTCTVSGVSLPDYGV |
| | leader | SWIRQPPRKGLEWLGVIWGSETTYYNSALKSRLTIIKDNSKSQVFLKMNSLQT |
| | | DDTAIYYCAKHYYYGGSYAMDYWGQGTSVTVSS |

Murine CART19

| | | |
|---|---|---|
| SEQ ID | HCDR1 | DYGVS |
| NO: 1094 | (Kabat) | |
| SEQ ID | HCDR2 | VIWGSETTYYNSALKS |
| NO: 1095 | (Kabat) | |
| SEQ ID | HCDR3 | HYYYGGSYAMDY |
| NO: 1096 | (Kabat) | |
| SEQ ID | LCDR1 | RASQDISKYLN |
| NO: 1097 | (Kabat) | |
| SEQ ID | LCDR2 | HTSRLHS |
| NO: 1098 | (Kabat) | |
| SEQ ID | LCDR3 | QQGNTLPYT |
| NO: 1099 | (Kabat) | |

Humanized CART19 a

| | | |
|---|---|---|
| SEQ ID | HCDR1 | DYGVS |
| NO: 1100 | (Kabat) | |
| SEQ ID | HCDR2 | VIWGSETTYYSSSLKS |
| NO: 1101 | (Kabat) | |
| SEQ ID | HCDR3 | HYYYGGSYAMDY |
| NO: 1102 | (Kabat) | |
| SEQ ID | LCDR1 | RASQDISKYLN |
| NO: 1103 | (Kabat) | |
| SEQ ID | LCDR2 | HTSRLHS |
| NO: 1104 | (Kabat) | |
| SEQ ID | LCDR3 | QQGNTLPYT |
| NO: 1105 | (Kabat) | |

Humanized CART19 b

| | | |
|---|---|---|
| SEQ ID | HCDR1 | DYGVS |
| NO: 1106 | (Kabat) | |
| SEQ ID | HCDR2 | VIWGSETTYYQSSLKS |
| NO: 1107 | (Kabat) | |
| SEQ ID | HCDR3 | HYYYGGSYAMDY |
| NO: 1108 | (Kabat) | |
| SEQ ID | LCDR1 | RASQDISKYLN |
| NO: 1109 | (Kabat) | |
| SEQ ID | LCDR2 | HTSRLHS |
| NO: 1110 | (Kabat) | |
| SEQ ID | LCDR3 | QQGNTLPYT |
| NO: 1111 | (Kabat) | |

Humanized CART19 c

| | | |
|---|---|---|
| SEQ ID | HCDR1 | DYGVS |
| NO: 1112 | (Kabat) | |
| SEQ ID | HCDR2 | VIWGSETTYYNSSLKS |
| NO: 1113 | (Kabat) | |
| SEQ ID | HCDR3 | HYYYGGSYAMDY |
| NO: 1114 | (Kabat) | |
| SEQ ID | LCDR1 | RASQDISKYLN |
| NO: 1115 | (Kabat) | |
| SEQ ID | LCDR2 | HTSRLHS |
| NO: 1116 | (Kabat) | |
| SEQ ID | LCDR3 | QQGNTLPYT |
| NO: 1117 | (Kabat) | |

CD19 CAR constructs containing humanized anti-CD19 scFv domains are described in PCT publication WO 2014/153270, incorporated herein by reference.

The sequences of murine and humanized CDR sequences of the anti-CD19 scFv domains are shown in Table 4 for the heavy chain variable domains and in Table 5 for the light chain variable domains. The SEQ ID NOs refer to those found in Table 3.

TABLE 4
Exemplary Heavy Chain Variable Domain CDR
(Kabat) SEQ ID NO's of CD19 Antibodies

| Candidate | HCDR1 | HCDR2 | HCDR3 |
|---|---|---|---|
| murine_CART19 | SEQ ID NO: 1094 | SEQ ID NO: 1095 | SEQ ID NO: 1096 |
| humanized_CART19 a | SEQ ID NO: 1100 | SEQ ID NO: 1101 | SEQ ID NO: 1102 |
| humanized_CART19 b | SEQ ID NO: 1106 | SEQ ID NO: 1107 | SEQ ID NO: 1108 |
| humanized_CART19 c | SEQ ID NO: 1112 | SEQ ID NO: 1113 | SEQ ID NO: 1114 |

TABLE 5
Exemplary Light Chain Variable Domain CDR
(Kabat) SEQ ID NO's of CD19 Antibodies

| Candidate | LCDR1 | LCDR2 | LCDR3 |
|---|---|---|---|
| murine_CART19 | SEQ ID NO: 1097 | SEQ ID NO: 1098 | SEQ ID NO: 1099 |
| humanized_CART19 a | SEQ ID NO: 1103 | SEQ ID NO: 1104 | SEQ ID NO: 1105 |
| humanized_CART19 b | SEQ ID NO: 1109 | SEQ ID NO: 1110 | SEQ ID NO: 1111 |
| humanized_CART19 c | SEQ ID NO: 1115 | SEQ ID NO: 1116 | SEQ ID NO: 1117 |

Any known CD19 CAR, e.g., the CD19 antigen-binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Exemplary CD19 CARs include CD19 CARs described herein, e.g., in one or more tables described herein, or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

### BCMA CAR and BCMA-Binding Sequences

In some embodiments, the CAR or CAR-expressing cell described herein is a BCMA CAR-expressing cell (e.g., a cell expressing a CAR molecule that binds to human BCMA). Exemplary BCMA CAR molecules can include sequences disclosed in Table 1 or 16 of WO2016/014565, incorporated herein by reference. The BCMA CAR construct can include an optional leader sequence; an optional hinge domain, e.g., a CD8 hinge domain; a transmembrane domain, e.g., a CD8 transmembrane domain; an intracellular domain, e.g., a 4-1BB intracellular domain; and a functional signaling domain, e.g., a CD3 zeta domain. In some embodiments, the domains are contiguous and in the same reading frame to form a single fusion protein. In other embodiments, the domain are in separate polypeptides, e.g., as in an RCAR molecule as described herein.

The sequences of exemplary BCMA CAR molecules or fragments thereof are disclosed in Tables 6-8. In some embodiments, the full length BCMA CAR molecule includes one or more CDRs, VH, VL, scFv, or full-length sequences of, BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1, as disclosed in Tables U, V, W, and X, or a sequence substantially (e.g., 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

TABLE 6
Amino Acid and Nucleic Acid Sequences of Exemplary Anti-BCMA scFv Domains and
BCMA CAR Molecules. The amino acid sequences variable heavy chain and variable
light chain sequences for each scFv is also provided.

| | | |
|---|---|---|
| | SEQ | |
| Name/ | ID | |
| Description | NO: | Sequence |

139109

| | | |
|---|---|---|
| 139109-aa | 49 | EVQLVESGGGLVQPGGSLRLSCAVSGFALSNHGMSWVRRAPGKGLE |
| ScFv domain | | WVSGIVYSGSTYYAASVKGRFTISRDNSRNTLYLQMNSLRPEDTAIYY |
| | | CSAHGGESDVWGQGTTVTVSSASGGGGSGGRASGGGGSDIQLTQSPSS |
| | | LSASVGDRVTITCRASQSISSYLNWYQQKPGKAPKLLIYAASSLQSGVP |
| | | SRFSGSGSGTDFTLTISSLQPEDFATYYCQQSYSTPYTFGQGTKVEIK |
| 139109-nt | 64 | GAAGTGCAATTGGTGGAATCAGGGGGAGGACTTGTGCAGCCTGGA |
| ScFv domain | | GGATCGCTGAGACTGTCATGTGCCGTGTCCGGCTTTGCCCTGTCCAA |
| | | CCACGGGATGTCCTGGGTCCGCCGCGCGCCTGGAAAGGGCCTCGAA |
| | | TGGGTGTCGGGTATTGTGTACAGCGGTAGCACCTACTATGCCGCAT |
| | | CCGTGAAGGGGAGATTCACCATCAGCCGGGACAACTCCAGGAACA |
| | | CTCTGTACCTCCAAATGAATTCGCTGAGGCCAGAGGACACTGCCAT |
| | | CTACTACTGCTCCGCGCATGGCGGAGAGTCCGACGTCTGGGGACAG |
| | | GGGACCACCGTGACCGTGTCTAGCGCGTCCGGCGGAGGCGGCAGC |
| | | GGGGGTCGGGCATCAGGGGGCGGCGGATCGGACATCCAGCTCACC |
| | | CAGTCCCCGAGCTCGCTGTCCGCCTCCGTGGGAGATCGGGTCACCA |
| | | TCACGTGCCGCGCCAGCCAGTCGATTTCCTCCTACCTGAACTGGTAC |
| | | CAACAGAAGCCCGGAAAAGCCCCGAAGCTTCTCATCTACGCCGCCT |
| | | CGAGCCTGCAGTCAGGAGTGCCCTCACGGTTCTCCGGCTCCGGTTC |
| | | CGGTACTGATTTCACCCTGACCATTTCCTCCCTGCAACCGGAGGACT |
| | | TCGCTACTTACTACTGCCAGCAGTCGTACTCCACCCCCTACACTTTC |
| | | GGACAAGGCACCAAGGTCGAAATCAAG |
| 139109-aa | 79 | EVQLVESGGGLVQPGGSLRLSCAVSGFALSNHGMSWVRRAPGKGLE |
| VH | | WVSGIVYSGSTYYAASVKGRFTISRDNSRNTLYLQMNSLRPEDTAIYY |
| | | CSAHGGESDVWGQGTTVTVSS |
| 139109-aa | 94 | DIQLTQSPSSLSASVGDRVTITCRASQSISSYLNWYQQKPGKAPKLLIYA |
| VL | | ASSLQSGVPSRFSGSGSGTDFTLTISSLQPEDFATYYCQQSYSTPYTFGQ |
| | | GTKVEIK |
| 139109-aa | 109 | MALPVTALLLPLALLLHAARPEVQLVESGGGLVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSDIQLTQSPSSLSASVGDRVTITCRASQSISSYLNWYQQ |
| | | KPGKAPKLLIYAASSLQSGVPSRFSGSGSGTDFTLTISSLQPEDFATYYC |
| | | QQSYSTPYTFGQGTKVEIKTTTPAPRPPTPAPTIASQPLSLRPEACRPAA |
| | | GGAVHTRGLDFACDIYIWAPLAGTCGVLLLSLVITLYCKRGRKKLLYIF |
| | | KQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQ |
| | | KDKMAEAYSEIGMKGERRRGKGHDGLYQGLSTATKDTYDALHMQAL |
| | | PPR |
| 139109-nt | 124 | ATGGCCCTCCCTGTCACCGCCCTGCTGCTTCCGCTGGCTCTTCTGCT |
| Full CAR | | CCACGCCGCTCGGCCCGAAGTGCAATTGGTGGAATCAGGGGGAGG |
| | | ACTTGTGCAGCCTGGAGGATCGCTGAGACTGTCATGTGCCGTGTCC |
| | | GGCTTTGCCCTGTCCAACCACGGGATGTCCTGGGTCCGCCGCGCGC |
| | | CTGGAAAGGGCCTCGAATGGGTGTCGGGTATTGTGTACAGCGGTAG |
| | | CACCTACTATGCCGCATCCGTGAAGGGGAGATTCACCATCAGCCGG |
| | | GACAACTCCAGGAACACTCTGTACCTCCAAATGAATTCGCTGAGGC |
| | | CAGAGGACACTGCCATCTACTACTGCTCCGCGCATGGCGGAGAGTC |
| | | CGACGTCTGGGGACAGGGGACCACCGTGACCGTGTCTAGCGCGTCC |
| | | GGCGGAGGCGGCAGCGGGGGTCGGGCATCAGGGGGCGGCGGATCG |
| | | GACATCCAGCTCACCCAGTCCCCGAGCTCGCTGTCCGCCTCCGTGG |
| | | GAGATCGGGTCACCATCACGTGCCGCGCCAGCCAGTCGATTTCCTC |
| | | CTACCTGAACTGGTACCAACAGAAGCCCGGAAAAGCCCCGAAGCTT |
| | | CTCATCTACGCCGCCTCGAGCCTGCAGTCAGGAGTGCCCTCACGGT |
| | | TCTCCGGCTCCGGTTCCGGTACTGATTTCACCCTGACCATTTCCTCC |
| | | CTGCAACCGGAGGACTTCGCTACTTACTACTGCCAGCAGTCGTACT |
| | | CCACCCCCTACACTTTCGGACAAGGCACCAAGGTCGAAATCAAGAC |
| | | CACTACCCCAGCACCGAGGCCACCCACCCCGGCTCCTACCATCGCC |
| | | TCCCAGCCTCTGTCCCTGCGTCCGGAGGCATGTAGACCCGCAGCTG |
| | | GTGGGGCCGTGCATACCCGGGGTCTTGACTTCGCCTGCGATATCTA |
| | | CATTTGGGCCCCTCTGGCTGGTACTTGCGGGGTCCTGCTGCTTTCAC |
| | | TCGTGATCACTCTTTACTGTAAGCGCGGTCGGAAGAAGCTGCTGTA |
| | | CATCTTTAAGCAACCCTTCATGAGGCCTGTGCAGACTACTCAAGAG |
| | | GAGGACGGCTGTTCATGCCGGTTCCCAGAGGAGGAGGAAGGCGGC |
| | | TGCGAACTGCGCGTGAAATTCAGCCGCAGCGCAGATGCTCCAGCCT |
| | | ACAAGCAGGGGCAGAACCAGCTCTACAACGAACTCAATCTTGGTCG |
| | | GAGAGAGGAGTACGACGTGCTGGACAAGCGGAGAGGACGGGACCC |
| | | AGAAATGGGCGGGAAGCCGCGCAGAAAGAATCCCCAAGAGGGCCT |
| | | GTACAACGAGCTCCAAAAGGATAAGATGGCAGAAGCCTATAGCGA |
| | | GATTGGTATGAAAGGGGAACGCAGAAGAGGCAAAGGCCACGACGG |
| | | ACTGTACCAGGGACTCAGCACCGCCACCAAGGACACCTATGACGCT |
| | | CTTCACATGCAGGCCCTGCCGCCTCGG |
| Full CAR | 392 | EVQLVESGGGLVQPGGSLRLSCAVSGFALSNHGMSWVRRAPGKGLE |
| without | | WVSGIVYSGSTYYAASVKGRFTISRDNSRNTLYLQMNSLRPEDTAIYY |
| leader | | CSAHGGESDVWGQGTTVTVSSASGGGGSGGRASGGGGSDIQLTQSPSS |
| sequence | | LSASVGDRVTITCRASQSISSYLNWYQQKPGKAPKLLIYAASSLQSGVP |
| | | SRFSGSGSGTDFTLTISSLQPEDFATYYCQQSYSTPYTFGQGTKVEIKTT |
| | | TPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRF |
| | | PEEEEGGCELRVKFSRSADAPAYKQGQNQLYNELNLGRREEYDVLDK |
| | | RRGRDPEMGGKPRRKNPQEGLYNELQKDKMAEAYSEIGMKGERRRG |
| | | KGHDGLYQGLSTATKDTYDALHMQALPPR |
| Full CAR | 393 | EVQLVESGGGLVQPGGSLRLSCAVSGFALSNHGMSWVRRAPGKGLE |
| without | | WVSGIVYSGSTYYAASVKGRFTISRDNSRNTLYLQMNSLRPEDTAIYY |
| linker, | | CSAHGGESDVWGQGTTVTVSSDIQLTQSPSSLSASVGDRVTITCRASQS |
| without | | ISSYLNWYQQKPGKAPKLLIYAASSLQSGVPSRFSGSGSGTDFTLTISSL |
| leader | | QPEDFATYYCQQSYSTPYTFGQGTKVEIKTTTPAPRPPTPAPTIASQPLS |
| sequence | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKN |
| | | PQEGLYNELQKDKMAEAYSEIGMKGERRRGKGHDGLYQGLSTATKD |
| | | TYDALHMQALPPR |

139103

| | | |
|---|---|---|
| 139103-aa | 39 | QVQLVESGGGLVQPGRSLRLSCAASGFTFSNYAMSWVRQAPGKGLG |
| ScFv domain | | WVSGISRSGENTYYADSVKGRFTISRDNSKNTLYLQMNSLRDEDTAVY |
| | | YCARSPAHYYGGMDVWGQGTTVTVSSASGGGGSGGRASGGGGSDIV |
| | | LTQSPGTLSLSPGERATLSCRASQSISSSFLAWYQQKPGQAPRLLIYGAS |
| | | RRATGIPDRFSGSGSGTDFTLTISRLEPEDSAVYYCQQYHSSPSWTFGQ |
| | | GTKLEIK |
| 139103-nt | 54 | CAAGTGCAACTCGTGGAATCTGGTGGAGGACTCGTGCAACCCGGAA |
| ScFv domain | | GATCGCTTAGACTGTCGTGTGCCGCCAGCGGGTTCACTTTCTCGAAC |
| | | TACGCGATGTCCTGGGTCCGCCAGGCACCCGGAAAGGGACTCGGTT |
| | | GGGTGTCCGGCATTTCCCGGTCCGGCGAAAATACCTACTACGCCGA |
| | | CTCCGTGAAGGGCCGCTTCACCATCTCAAGGGACAACAGCAAAAAC |
| | | ACCCTGTACTTGCAAATGAACTCCCTGCGGGATGAAGATACAGCCG |
| | | TGTACTATTGCGCCCGGTCGCCTGCCCATTACTACGGCGGAATGGA |
| | | CGTCTGGGGACAGGGAACCACTGTGACTGTCAGCAGCGCGTCGGGT |
| | | GGCGGCGGCTCAGGGGGTCGGGCCTCCGGGGGGGGAGGGTCCGAC |
| | | ATCGTGCTGACCCAGTCCCCGGGAACCCTGAGCCTGAGCCCGGGAG |
| | | AGCGCGCGACCCTGTCATGCCGGGCATCCCAGAGCATTAGCTCCTC |
| | | CTTTCTCGCCTGGTATCAGCAGAAGCCCGGACAGGCCCCGAGGCTG |
| | | CTGATCTACGGCGCTAGCAGAAGGGCTACCGGAATCCCAGACCGGT |
| | | TCTCCGGCTCCGGTTCCGGGACCGATTTCACCCTTACTATCTCGCGC |
| | | CTGGAACCTGAGGACTCCGCCGTCTACTACTGCCAGCAGTACCACT |
| | | CATCCCCGTCGTGGACGTTCGGACAGGGCACCAAGCTGGAGATTAA |
| | | G |
| 139103-aa | 69 | QVQLVESGGGLVQPGRSLRLSCAASGFTFSNYAMSWVRQAPGKGLG |
| VH | | WVSGISRSGENTYYADSVKGRFTISRDNSKNTLYLQMNSLRDEDTAVY |
| | | YCARSPAHYYGGMDVWGQGTTVTVSS |
| 139103-aa | 84 | DIVLTQSPGTLSLSPGERATLSCRASQSISSSFLAWYQQKPGQAPRLLIY |
| VL | | GASRRATGIPDRFSGSGSGTDFTLTISRLEPEDSAVYYCQQYHSSPSWTF |
| | | GQGTKLEIK |
| 139103-aa | 99 | MALPVTALLLPLALLLHAARPQVQLVESGGGLVQPGRSLRLSCAASGF |
| Full CAR | | TFSNYAMSWVRQAPGKGLGWVSGISRSGENTYYADSVKGRFTISRDN |
| | | SKNTLYLQMNSLRDEDTAVYYCARSPAHYYGGMDVWGQGTTVTVSS |
| | | ASGGGGSGGRASGGGGSDIVLTQSPGTLSLSPGERATLSCRASQSISSSF |
| | | LAWYQQKPGQAPRLLIYGASRRATGIPDRFSGSGSGTDFTLTISRLEPE |
| | | DSAVYYCQQYHSSPSWTFGQGTKLEIKTTTPAPRPPTPAPTIASQPLSLR |
| | | PEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSAD |
| | | APAYKQGQNQLYNELNLGRREEYDVLDKRRGRDPEMGGKPRRKNPQ |
| | | EGLYNELQKDKMAEAYSEIGMKGERRRGKGHDGLYQGLSTATKDTY |
| | | DALHMQALPPR |
| 139103-nt | 114 | ATGGCCCTCCCTGTCACCGCCCTGCTGCTTCCGCTGGCTCTTCTGCT |
| Full CAR | | CCACGCCGCTCGGCCCCAAGTGCAACTCGTGGAATCTGGTGGAGGA |
| | | CTCGTGCAACCCGGAAGATCGCTTAGACTGTCGTGTGCCGCCAGCG |
| | | GGTTCACTTTCTCGAACTACGCGATGTCCTGGGTCCGCCAGGCACCC |
| | | GGAAAGGGACTCGGTTGGGTGTCCGGCATTTCCCGGTCCGGCGAAA |
| | | ATACCTACTACGCCGACTCCGTGAAGGGCCGCTTCACCATCTCAAG |
| | | GGACAACAGCAAAAACACCCTGTACTTGCAAATGAACTCCCTGCGG |
| | | GATGAAGATACAGCCGTGTACTATTGCGCCCGGTCGCCTGCCCATT |
| | | ACTACGGCGGAATGGACGTCTGGGGACAGGGAACCACTGTGACTGT |
| | | CAGCAGCGCGTCGGGTGGCGGCGGCTCAGGGGGTCGGGCCTCCGG |
| | | GGGGGGAGGGTCCGACATCGTGCTGACCCAGTCCCCGGGAACCCTG |
| | | AGCCTGAGCCCGGGAGAGCGCGCGACCCTGTCATGCCGGGCATCCC |
| | | AGAGCATTAGCTCCTCCTTTCTCGCCTGGTATCAGCAGAAGCCCGG |
| | | ACAGGCCCCGAGGCTGCTGATCTACGGCGCTAGCAGAAGGGCTACC |
| | | GGAATCCCAGACCGGTTCTCCGGCTCCGGTTCCGGGACCGATTTCA |
| | | CCCTTACTATCTCGCGCCTGGAACCTGAGGACTCCGCCGTCTACTAC |
| | | TGCCAGCAGTACCACTCATCCCCGTCGTGGACGTTCGGACAGGGCA |
| | | CCAAGCTGGAGATTAAGACCACTACCCCAGCACCGAGGCCACCCAC |
| | | CCCGGCTCCTACCATCGCCTCCCAGCCTCTGTCCCTGCGTCCGGAGG |
| | | CATGTAGACCCGCAGCTGGTGGGGCCGTGCATACCCGGGGTCTTGA |
| | | CTTCGCCTGCGATATCTACATTTGGGCCCCTCTGGCTGGTACTTGCG |
| | | GGGTCCTGCTGCTTTCACTCGTGATCACTCTTTACTGTAAGCGCGGT |
| | | CGGAAGAAGCTGCTGTACATCTTTAAGCAACCCTTCATGAGGCCTG |
| | | TGCAGACTACTCAAGAGGAGGACGGCTGTTCATGCCGGTTCCCAGA |
| | | GGAGGAGGAAGGCGGCTGCGAACTGCGCGTGAAATTCAGCCGCAG |
| | | CGCAGATGCTCCAGCCTACAAGCAGGGGCAGAACCAGCTCTACAAC |
| | | GAACTCAATCTTGGTCGGAGAGAGGAGTACGACGTGCTGGACAAG |
| | | CGGAGAGGACGGGACCCAGAAATGGGCGGGAAGCCGCGCAGAAA |
| | | GAATCCCCAAGAGGGCCTGTACAACGAGCTCCAAAAGGATAAGAT |
| | | GGCAGAAGCCTATAGCGAGATTGGTATGAAAGGGGAACGCAGAAG |
| | | AGGCAAAGGCCACGACGGACTGTACCAGGGACTCAGCACCGCCAC |
| | | CAAGGACACCTATGACGCTCTTCACATGCAGGCCCTGCCGCCTCGG |

139105

| | | |
|---|---|---|
| 139105-aa | 40 | QVQLVESGGGLVQPGRSLRLSCAASGFTFDDYAMHWVRQAPGKGLE |
| ScFv domain | | WVSGISWNSGSIGYADSVKGRFTISRDNAKNSLYLQMNSLRAEDTALY |
| | | YCSVHSFLAYWGQGTLVTVSSASGGGGSGGRASGGGGSDIVMTQTPL |
| | | SLPVTPGEPASISCRSSQSLLHSNGYNYLDWYLQKPGQSPQLLIYLGSN |
| | | RASGVPDRFSGSGSGTDFTLKISRVEAEDVGVYYCMQALQTPYTFGQG |
| | | TKVEIK |
| 139105-nt | 55 | CAAGTGCAACTCGTCGAATCCGGTGGAGGTCTGGTCCAACCTGGTA |
| ScFv domain | | GAAGCCTGAGACTGTCGTGTGCGGCCAGCGGATTCACCTTTGATGA |
| | | CTATGCTATGCACTGGGTGCGGCAGGCCCCAGGAAAGGGCCTGGAA |
| | | TGGGTGTCGGGAATTAGCTGGAACTCCGGGTCCATTGGCTACGCCG |
| | | ACTCCGTGAAGGGCCGCTTCACCATCTCCCGCGACAACGCAAAGAA |
| | | CTCCCTGTACTTGCAAATGAACTCGCTCAGGGCTGAGGATACCGCG |
| | | CTGTACTACTGCTCCGTGCATTCCTTCCTGGCCTACTGGGGACAGGG |
| | | AACTCTGGTCACCGTGTCGAGCGCCTCCGGCGGCGGGGGCTCGGGT |
| | | GGACGGGCCTCGGGCGGAGGGGGGTCCGACATCGTGATGACCCAG |
| | | ACCCCGCTGAGCTTGCCCGTGACTCCCGGAGAGCCTGCATCCATCT |
| | | CCTGCCGGTCATCCCAGTCCCTTCTCCACTCCAACGGATACAACTAC |
| | | CTCGACTGGTACCTCCAGAAGCCGGGACAGAGCCCTCAGCTTCTGA |
| | | TCTACCTGGGGTCAAATAGAGCCTCAGGAGTGCCGGATCGGTTCAG |
| | | CGGATCTGGTTCGGGAACTGATTTCACTCTGAAGATTTCCCGCGTGG |
| | | AAGCCGAGGACGTGGGCGTCTACTACTGTATGCAGGCGCTGCAGAC |
| | | CCCCTATACCTTCGGCCAAGGGACGAAAGTGGAGATCAAG |
| 139105-aa | 70 | QVQLVESGGGLVQPGRSLRLSCAASGFTFDDYAMHWVRQAPGKGLE |
| VH | | WVSGISWNSGSIGYADSVKGRFTISRDNAKNSLYLQMNSLRAEDTALY |
| | | YCSVHSFLAYWGQGTLVTVSS |
| 139105-aa | 85 | DIVMTQTPLSLPVTPGEPASISCRSSQSLLHSNGYNYLDWYLQKPGQSP |
| VL | | QLLIYLGSNRASGVPDRFSGSGSGTDFTLKISRVEAEDVGVYYCMQAL |
| | | QTPYTFGQGTKVEIK |
| 139105-aa | 100 | MALPVTALLLPLALLLHAARPQVQLVESGGGLVQPGRSLRLSCAASGF |
| Full CAR | | TFDDYAMHWVRQAPGKGLEWVSGISWNSGSIGYADSVKGRFTISRDN |
| | | AKNSLYLQMNSLRAEDTALYYCSVHSFLAYWGQGTLVTVSSASGGGG |
| | | SGGRASGGGGSDIVMTQTPLSLPVTPGEPASISCRSSQSLLHSNGYNYL |
| | | DWYLQKPGQSPQLLIYLGSNRASGVPDRFSGSGSGTDFTLKISRVEAED |
| | | VGVYYCMQALQTPYTFGQGTKVEIKTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYD |
| | | ALHMQALPPR |
| 139105-nt | 115 | ATGGCCCTCCCTGTCACCGCCCTGCTGCTTCCGCTGGCTCTTCTGCT |
| Full CAR | | CCACGCCGCTCGGCCCCAAGTGCAACTCGTCGAATCCGGTGGAGGT |
| | | CTGGTCCAACCTGGTAGAAGCCTGAGACTGTCGTGTGCGGCCAGCG |
| | | GATTCACCTTTGATGACTATGCTATGCACTGGGTGCGGCAGGCCCC |
| | | AGGAAAGGGCCTGGAATGGGTGTCGGGAATTAGCTGGAACTCCGG |
| | | GTCCATTGGCTACGCCGACTCCGTGAAGGGCCGCTTCACCATCTCCC |
| | | GCGACAACGCAAAGAACTCCCTGTACTTGCAAATGAACTCGCTCAG |
| | | GGCTGAGGATACCGCGCTGTACTACTGCTCCGTGCATTCCTTCCTGG |
| | | CCTACTGGGGACAGGGAACTCTGGTCACCGTGTCGAGCGCCTCCGG |
| | | CGGCGGGGGCTCGGGTGGACGGGCCTCGGGCGGAGGGGGGTCCGA |
| | | CATCGTGATGACCCAGACCCCGCTGAGCTTGCCCGTGACTCCCGGA |
| | | GAGCCTGCATCCATCTCCTGCCGGTCATCCCAGTCCCTTCTCCACTC |
| | | CAACGGATACAACTACCTCGACTGGTACCTCCAGAAGCCGGGACAG |
| | | AGCCCTCAGCTTCTGATCTACCTGGGGTCAAATAGAGCCTCAGGAG |
| | | TGCCGGATCGGTTCAGCGGATCTGGTTCGGGAACTGATTTCACTCTG |
| | | AAGATTTCCCGCGTGGAAGCCGAGGACGTGGGCGTCTACTACTGTA |
| | | TGCAGGCGCTGCAGACCCCCTATACCTTCGGCCAAGGGACGAAAGT |
| | | GGAGATCAAGACCACTACCCCAGCACCGAGGCCACCCACCCCGGCT |
| | | CCTACCATCGCCTCCCAGCCTCTGTCCCTGCGTCCGGAGGCATGTAG |
| | | ACCCGCAGCTGGTGGGGCCGTGCATACCCGGGGTCTTGACTTCGCC |
| | | TGCGATATCTACATTTGGGCCCCTCTGGCTGGTACTTGCGGGGTCCT |
| | | GCTGCTTTCACTCGTGATCACTCTTTACTGTAAGCGCGGTCGGAAGA |
| | | AGCTGCTGTACATCTTTAAGCAACCCTTCATGAGGCCTGTGCAGACT |
| | | ACTCAAGAGGAGGACGGCTGTTCATGCCGGTTCCCAGAGGAGGAG |
| | | GAAGGCGGCTGCGAACTGCGCGTGAAATTCAGCCGCAGCGCAGAT |
| | | GCTCCAGCCTACAAGCAGGGGCAGAACCAGCTCTACAACGAACTCA |
| | | ATCTTGGTCGGAGAGAGGAGTACGACGTGCTGGACAAGCGGAGAG |
| | | GACGGGACCCAGAAATGGGCGGGAAGCCGCGCAGAAAGAATCCCC |
| | | AAGAGGGCCTGTACAACGAGCTCCAAAAGGATAAGATGGCAGAAG |
| | | CCTATAGCGAGATTGGTATGAAAGGGGAACGCAGAAGAGGCAAAG |
| | | GCCACGACGGACTGTACCAGGGACTCAGCACCGCCACCAAGGACA |
| | | CCTATGACGCTCTTCACATGCAGGCCCTGCCGCCTCGG |

139111

| | | |
|---|---|---|
| 139111-aa | 41 | EVQLLESGGGLVQPGGSLRLSCAVSGFALSNHGMSWVRRAPGKGLEW |
| ScFv domain | | VSGIVYSGSTYYAASVKGRFTISRDNSRNTLYLQMNSLRPEDTAIYYCS |
| | | AHGGESDVWGQGTTVTVSSASGGGGSGGRASGGGGSDIVMTQTPLSL |
| | | SVTPGQPASISCKSSQSLLRNDGKTPLYWYLQKAGQPPQLLIYEVSNRF |
| | | SGVPDRFSGSGSGTDFTLKISRVEAEDVGAYYCMQNIQFPSFGGGTKLE |
| | | IK |
| 139111-nt | 56 | GAAGTGCAATTGTTGGAATCTGGAGGAGGACTTGTGCAGCCTGGAG |
| ScFv domain | | GATCACTGAGACTTTCGTGTGCGGTGTCAGGCTTCGCCCTGAGCAA |
| | | CCACGGCATGAGCTGGGTGCGGAGAGCCCCGGGGAAGGGTCTGGA |
| | | ATGGGTGTCCGGGATCGTCTACTCCGGTTCAACTTACTACGCCGCA |
| | | AGCGTGAAGGGTCGCTTCACCATTTCCCGCGATAACTCCCGGAACA |
| | | CCCTGTACCTCCAAATGAACTCCCTGCGGCCCGAGGACACCGCCAT |
| | | CTACTACTGTTCCGCGCATGGAGGAGAGTCCGATGTCTGGGGACAG |
| | | GGCACTACCGTGACCGTGTCGAGCGCCTCGGGGGGAGGAGGCTCCG |
| | | GCGGTCGCGCCTCCGGGGGGGGTGGCAGCGACATTGTGATGACGCA |
| | | GACTCCACTCTCGCTGTCCGTGACCCCGGGACAGCCCGCGTCCATCT |
| | | CGTGCAAGAGCTCCCAGAGCCTGCTGAGGAACGACGGAAAGACTC |
| | | CTCTGTATTGGTACCTCCAGAAGGCTGGACAGCCCCCGCAACTGCT |
| | | CATCTACGAAGTGTCAAATCGCTTCTCCGGGGTGCCGGATCGGTTTT |
| | | CCGGCTCGGGATCGGGCACCGACTTCACCCTGAAAATCTCCAGGGT |
| | | CGAGGCCGAGGACGTGGGAGCCTACTACTGCATGCAAAACATCCA |
| | | GTTCCCTTCCTTCGGCGGCGGCACAAAGCTGGAGATTAAG |
| 139111-aa | 71 | EVQLLESGGGLVQPGGSLRLSCAVSGFALSNHGMSWVRRAPGKGLEW |
| VH | | VSGIVYSGSTYYAASVKGRFTISRDNSRNTLYLQMNSLRPEDTAIYYCS |
| | | AHGGESDVWGQGTTVTVSS |
| 139111-aa | 86 | DIVMTQTPLSLSVTPGQPASISCKSSQSLLRNDGKTPLYWYLQKAGQPP |
| VL | | QLLIYEVSNRFSGVPDRFSGSGSGTDFTLKISRVEAEDVGAYYCMQNIQ |
| | | FPSFGGGTKLEIK |
| 139111-aa | 101 | MALPVTALLLPLALLLHAARPEVQLLESGGGLVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSDIVMTQTPLSLSVTPGQPASISCKSSQSLLRNDGKTPL |
| | | YWYLQKAGQPPQLLIYEVSNRFSGVPDRFSGSGSGTDFTLKISRVEAED |
| | | VGAYYCMQNIQFPSFGGGTKLEIKTTTPAPRPPTPAPTIASQPLSLRPEA |
| | | CRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGL |
| | | YNELQKDKMAEAYSEIGMKGERRRGKGHDGLYQGLSTATKDTYDAL |
| | | HMQALPPR |
| 139111-nt | 116 | ATGGCCCTCCCTGTCACCGCCCTGCTGCTTCCGCTGGCTCTTCTGCT |
| Full CAR | | CCACGCCGCTCGGCCCGAAGTGCAATTGTTGGAATCTGGAGGAGGA |
| | | CTTGTGCAGCCTGGAGGATCACTGAGACTTTCGTGTGCGGTGTCAG |
| | | GCTTCGCCCTGAGCAACCACGGCATGAGCTGGGTGCGGAGAGCCCC |
| | | GGGGAAGGGTCTGGAATGGGTGTCCGGGATCGTCTACTCCGGTTCA |
| | | ACTTACTACGCCGCAAGCGTGAAGGGTCGCTTCACCATTTCCCGCG |
| | | ATAACTCCCGGAACACCCTGTACCTCCAAATGAACTCCCTGCGGCC |
| | | CGAGGACACCGCCATCTACTACTGTTCCGCGCATGGAGGAGAGTCC |
| | | GATGTCTGGGGACAGGGCACTACCGTGACCGTGTCGAGCGCCTCGG |
| | | GGGGAGGAGGCTCCGGCGGTCGCGCCTCCGGGGGGGGTGGCAGCG |
| | | ACATTGTGATGACGCAGACTCCACTCTCGCTGTCCGTGACCCCGGG |
| | | ACAGCCCGCGTCCATCTCGTGCAAGAGCTCCCAGAGCCTGCTGAGG |
| | | AACGACGGAAAGACTCCTCTGTATTGGTACCTCCAGAAGGCTGGAC |
| | | AGCCCCCGCAACTGCTCATCTACGAAGTGTCAAATCGCTTCTCCGG |
| | | GGTGCCGGATCGGTTTTCCGGCTCGGGATCGGGCACCGACTTCACC |
| | | CTGAAAATCTCCAGGGTCGAGGCCGAGGACGTGGGAGCCTACTACT |
| | | GCATGCAAAACATCCAGTTCCCTTCCTTCGGCGGCGGCACAAAGCT |
| | | GGAGATTAAGACCACTACCCCAGCACCGAGGCCACCCACCCCGGCT |
| | | CCTACCATCGCCTCCCAGCCTCTGTCCCTGCGTCCGGAGGCATGTAG |
| | | ACCCGCAGCTGGTGGGGCCGTGCATACCCGGGGTCTTGACTTCGCC |
| | | TGCGATATCTACATTTGGGCCCCTCTGGCTGGTACTTGCGGGGTCCT |
| | | GCTGCTTTCACTCGTGATCACTCTTTACTGTAAGCGCGGTCGGAAGA |
| | | AGCTGCTGTACATCTTTAAGCAACCCTTCATGAGGCCTGTGCAGACT |
| | | ACTCAAGAGGAGGACGGCTGTTCATGCCGGTTCCCAGAGGAGGAG |
| | | GAAGGCGGCTGCGAACTGCGCGTGAAATTCAGCCGCAGCGCAGAT |
| | | GCTCCAGCCTACAAGCAGGGGCAGAACCAGCTCTACAACGAACTCA |
| | | ATCTTGGTCGGAGAGAGGAGTACGACGTGCTGGACAAGCGGAGAG |
| | | GACGGGACCCAGAAATGGGCGGGAAGCCGCGCAGAAAGAATCCCC |
| | | AAGAGGGCCTGTACAACGAGCTCCAAAAGGATAAGATGGCAGAAG |
| | | CCTATAGCGAGATTGGTATGAAAGGGGAACGCAGAAGAGGCAAAG |
| | | GCCACGACGGACTGTACCAGGGACTCAGCACCGCCACCAAGGACA |
| | | CCTATGACGCTCTTCACATGCAGGCCCTGCCGCCTCGG |

139100

| | | |
|---|---|---|
| 139100-aa | 42 | QVQLVQSGAEVRKTGASVKVSCKASGYIFDNFGINWVRQAPGQGLEW |
| ScFv domain | | MGWINPKNNNTNYAQKFQGRVTITADESTNTAYMEVSSLRSEDTAVY |
| | | YCARGPYYYQSYMDVWGQGTMVTVSSASGGGGSGGRASGGGGSDIV |
| | | MTQTPLSLPVTPGEPASISCRSSQSLLHSNGYNYLNWYLQKPGQSPQLL |
| | | IYLGSKRASGVPDRFSGSGSGTDFTLHITRVGAEDVGVYYCMQALQTP |
| | | YTFGQGTKLEIK |
| 139100-nt | 57 | CAAGTCCAACTCGTCCAGTCCGGCGCAGAAGTCAGAAAAACCGGTG |
| ScFv domain | | CTAGCGTGAAAGTGTCCTGCAAGGCCTCCGGCTACATTTTCGATAA |
| | | CTTCGGAATCAACTGGGTCAGACAGGCCCCGGGCCAGGGGCTGGA |
| | | ATGGATGGGATGGATCAACCCCAAGAACAACAACACCAACTACGC |
| | | ACAGAAGTTCCAGGGCCGCGTGACTATCACCGCCGATGAATCGACC |
| | | AATACCGCCTACATGGAGGTGTCCTCCCTGCGGTCGGAGGACACTG |
| | | CCGTGTATTACTGCGCGAGGGGCCCATACTACTACCAAAGCTACAT |
| | | GGACGTCTGGGGACAGGGAACCATGGTGACCGTGTCATCCGCCTCC |
| | | GGTGGTGGAGGCTCCGGGGGGCGGGCTTCAGGAGGCGGAGGAAGC |
| | | GATATTGTGATGACCCAGACTCCGCTTAGCCTGCCCGTGACTCCTGG |
| | | AGAACCGGCCTCCATTTCCTGCCGGTCCTCGCAATCACTCCTGCATT |
| | | CCAACGGTTACAACTACCTGAATTGGTACCTCCAGAAGCCTGGCCA |
| | | GTCGCCCCAGTTGCTGATCTATCTGGGCTCGAAGCGCGCCTCCGGG |
| | | GTGCCTGACCGGTTTAGCGGATCTGGGAGCGGCACGGACTTCACTC |
| | | TCCACATCACCCGCGTGGGAGCGGAGGACGTGGGAGTGTACTACTG |
| | | TATGCAGGCGCTGCAGACTCCGTACACATTCGGACAGGGCACCAAG |
| | | CTGGAGATCAAG |
| 139100-aa | 72 | QVQLVQSGAEVRKTGASVKVSCKASGYIFDNFGINWVRQAPGQGLEW |
| VH | | MGWINPKNNNTNYAQKFQGRVTITADESTNTAYMEVSSLRSEDTAVY |
| | | YCARGPYYYQSYMDVWGQGTMVTVSS |
| 139100-aa | 87 | DIVMTQTPLSLPVTPGEPASISCRSSQSLLHSNGYNYLNWYLQKPGQSP |
| VL | | QLLIYLGSKRASGVPDRFSGSGSGTDFTLHITRVGAEDVGVYYCMQAL |
| | | QTPYTFGQGTKLEIK |
| 139100-aa | 102 | MALPVTALLLPLALLLHAARPQVQLVQSGAEVRKTGASVKVSCKASG |
| Full CAR | | YIFDNFGINWVRQAPGQGLEWMGWINPKNNNTNYAQKFQGRVTITAD |
| | | ESTNTAYMEVSSLRSEDTAVYYCARGPYYYQSYMDVWGQGTMVTVS |
| | | SASGGGGSGGRASGGGGSDIVMTQTPLSLPVTPGEPASISCRSSQSLLHS |
| | | NGYNYLNWYLQKPGQSPQLLIYLGSKRASGVPDRFSGSGSGTDFTLHI |
| | | TRVGAEDVGVYYCMQALQTPYTFGQGTKLEIKTTTPAPRPPTPAPTIAS |
| | | QPLSLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELRVK |
| | | FSRSADAPAYKQGQNQLYNELNLGRREEYDVLDKRRGRDPEMGGKP |
| | | RRKNPQEGLYNELQKDKMAEAYSEIGMKGERRRGKGHDGLYQGLST |
| | | ATKDTYDALHMQALPPR |
| 139100-nt | 117 | ATGGCCCTCCCTGTCACCGCCCTGCTGCTTCCGCTGGCTCTTCTGCT |
| Full CAR | | CCACGCCGCTCGGCCCCAAGTCCAACTCGTCCAGTCCGGCGCAGAA |
| | | GTCAGAAAAACCGGTGCTAGCGTGAAAGTGTCCTGCAAGGCCTCCG |
| | | GCTACATTTTCGATAACTTCGGAATCAACTGGGTCAGACAGGCCCC |
| | | GGGCCAGGGGCTGGAATGGATGGGATGGATCAACCCCAAGAACAA |
| | | CAACACCAACTACGCACAGAAGTTCCAGGGCCGCGTGACTATCACC |
| | | GCCGATGAATCGACCAATACCGCCTACATGGAGGTGTCCTCCCTGC |
| | | GGTCGGAGGACACTGCCGTGTATTACTGCGCGAGGGGCCCATACTA |
| | | CTACCAAAGCTACATGGACGTCTGGGGACAGGGAACCATGGTGACC |
| | | GTGTCATCCGCCTCCGGTGGTGGAGGCTCCGGGGGGCGGGCTTCAG |
| | | GAGGCGGAGGAAGCGATATTGTGATGACCCAGACTCCGCTTAGCCT |
| | | GCCCGTGACTCCTGGAGAACCGGCCTCCATTTCCTGCCGGTCCTCGC |
| | | AATCACTCCTGCATTCCAACGGTTACAACTACCTGAATTGGTACCTC |
| | | CAGAAGCCTGGCCAGTCGCCCCAGTTGCTGATCTATCTGGGCTCGA |
| | | AGCGCGCCTCCGGGGTGCCTGACCGGTTTAGCGGATCTGGGAGCGG |
| | | CACGGACTTCACTCTCCACATCACCCGCGTGGGAGCGGAGGACGTG |
| | | GGAGTGTACTACTGTATGCAGGCGCTGCAGACTCCGTACACATTCG |
| | | GACAGGGCACCAAGCTGGAGATCAAGACCACTACCCCAGCACCGA |
| | | GGCCACCCACCCCGGCTCCTACCATCGCCTCCCAGCCTCTGTCCCTG |
| | | CGTCCGGAGGCATGTAGACCCGCAGCTGGTGGGGCCGTGCATACCC |
| | | GGGGTCTTGACTTCGCCTGCGATATCTACATTTGGGCCCCTCTGGCT |
| | | GGTACTTGCGGGGTCCTGCTGCTTTCACTCGTGATCACTCTTTACTG |
| | | TAAGCGCGGTCGGAAGAAGCTGCTGTACATCTTTAAGCAACCCTTC |
| | | ATGAGGCCTGTGCAGACTACTCAAGAGGAGGACGGCTGTTCATGCC |
| | | GGTTCCCAGAGGAGGAGGAAGGCGGCTGCGAACTGCGCGTGAAAT |
| | | TCAGCCGCAGCGCAGATGCTCCAGCCTACAAGCAGGGGCAGAACC |
| | | AGCTCTACAACGAACTCAATCTTGGTCGGAGAGAGGAGTACGACGT |
| | | GCTGGACAAGCGGAGAGGACGGGACCCAGAAATGGGCGGGAAGCC |
| | | GCGCAGAAAGAATCCCCAAGAGGGCCTGTACAACGAGCTCCAAAA |
| | | GGATAAGATGGCAGAAGCCTATAGCGAGATTGGTATGAAAGGGGA |
| | | ACGCAGAAGAGGCAAAGGCCACGACGGACTGTACCAGGGACTCAG |
| | | CACCGCCACCAAGGACACCTATGACGCTCTTCACATGCAGGCCCTG |
| | | CCGCCTCGG |

139101

| | | |
|---|---|---|
| 139101-aa | 43 | QVQLQESGGGLVQPGGSLRLSCAASGFTFSSDAMTWVRQAPGKGLEW |
| ScFv domain | | VSVISGSGGTTYYADSVKGRFTISRDNSKNTLYLQMNSLRAEDTAVYY |
| | | CAKLDSSGYYYARGPRYWGQGTLVTVSSASGGGGSGGRASGGGGSDI |
| | | QLTQSPSSLSASVGDRVTITCRASQSISSYLNWYQQKPGKAPKLLIYGA |
| | | STLASGVPARFSGSGSGTHFTLTINSLQSEDSATYYCQQSYKRASFGQG |
| | | TKVEIK |
| 139101-nt | 58 | CAAGTGCAACTTCAAGAATCAGGCGGAGGACTCGTGCAGCCCGGA |
| ScFv domain | | GGATCATTGCGGCTCTCGTGCGCCGCCTCGGGCTTCACCTTCTCGAG |
| | | CGACGCCATGACCTGGGTCCGCCAGGCCCCGGGGAAGGGGCTGGA |
| | | ATGGGTGTCTGTGATTTCCGGCTCCGGGGGAACTACGTACTACGCC |
| | | GATTCCGTGAAAGGTCGCTTCACTATCTCCCGGGACAACAGCAAGA |
| | | ACACCCTTTATCTGCAAATGAATTCCCTCCGCGCCGAGGACACCGC |
| | | CGTGTACTACTGCGCCAAGCTGGACTCCTCGGGCTACTACTATGCCC |
| | | GGGGTCCGAGATACTGGGGACAGGGAACCCTCGTGACCGTGTCCTC |
| | | CGCGTCCGGCGGAGGAGGGTCGGGAGGGCGGGCCTCCGGCGGCGG |
| | | CGGTTCGGACATCCAGCTGACCCAGTCCCCATCCTCACTGAGCGCA |
| | | AGCGTGGGCGACAGAGTCACCATTACATGCAGGGCGTCCCAGAGC |
| | | ATCAGCTCCTACCTGAACTGGTACCAACAGAAGCCTGGAAAGGCTC |
| | | CTAAGCTGTTGATCTACGGGGCTTCGACCCTGGCATCCGGGGTGCC |
| | | CGCGAGGTTTAGCGGAAGCGGTAGCGGCACTCACTTCACTCTGACC |
| | | ATTAACAGCCTCCAGTCCGAGGATTCAGCCACTTACTACTGTCAGC |
| | | AGTCCTACAAGCGGGCCAGCTTCGGACAGGGCACTAAGGTCGAGAT |
| | | CAAG |
| 139101-aa | 73 | QVQLQESGGGLVQPGGSLRLSCAASGFTFSSDAMTWVRQAPGKGLEW |
| VH | | VSVISGSGGTTYYADSVKGRFTISRDNSKNTLYLQMNSLRAEDTAVYY |
| | | CAKLDSSGYYYARGPRYWGQGTLVTVSS |
| 139101-aa | 88 | DIQLTQSPSSLSASVGDRVTITCRASQSISSYLNWYQQKPGKAPKLLIYG |
| VL | | ASTLASGVPARFSGSGSGTHFTLTINSLQSEDSATYYCQQSYKRASFGQ |
| | | GTKVEIK |
| 139101-aa | 103 | MALPVTALLLPLALLLHAARPQVQLQESGGGLVQPGGSLRLSCAASGF |
| Full CAR | | TFSSDAMTWVRQAPGKGLEWVSVISGSGGTTYYADSVKGRFTISRDNS |
| | | KNTLYLQMNSLRAEDTAVYYCAKLDSSGYYYARGPRYWGQGTLVTV |
| | | SSASGGGGSGGRASGGGGSDIQLTQSPSSLSASVGDRVTITCRASQSISS |
| | | YLNWYQQKPGKAPKLLIYGASTLASGVPARFSGSGSGTHFTLTINSLQS |
| | | EDSATYYCQQSYKRASFGQGTKVEIKTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYD |
| | | ALHMQALPPR |
| 139101-nt | 118 | ATGGCCCTCCCTGTCACCGCCCTGCTGCTTCCGCTGGCTCTTCTGCT |
| Full CAR | | CCACGCCGCTCGGCCCCAAGTGCAACTTCAAGAATCAGGCGGAGGA |
| | | CTCGTGCAGCCCGGAGGATCATTGCGGCTCTCGTGCGCCGCCTCGG |
| | | GCTTCACCTTCTCGAGCGACGCCATGACCTGGGTCCGCCAGGCCCC |
| | | GGGGAAGGGGCTGGAATGGGTGTCTGTGATTTCCGGCTCCGGGGGA |
| | | ACTACGTACTACGCCGATTCCGTGAAAGGTCGCTTCACTATCTCCCG |
| | | GGACAACAGCAAGAACACCCTTTATCTGCAAATGAATTCCCTCCGC |
| | | GCCGAGGACACCGCCGTGTACTACTGCGCCAAGCTGGACTCCTCGG |
| | | GCTACTACTATGCCCGGGGTCCGAGATACTGGGGACAGGGAACCCT |
| | | CGTGACCGTGTCCTCCGCGTCCGGCGGAGGAGGGTCGGGAGGGCG |
| | | GGCCTCCGGCGGCGGCGGTTCGGACATCCAGCTGACCCAGTCCCCA |
| | | TCCTCACTGAGCGCAAGCGTGGGCGACAGAGTCACCATTACATGCA |
| | | GGGCGTCCCAGAGCATCAGCTCCTACCTGAACTGGTACCAACAGAA |
| | | GCCTGGAAAGGCTCCTAAGCTGTTGATCTACGGGGCTTCGACCCTG |
| | | GCATCCGGGGTGCCCGCGAGGTTTAGCGGAAGCGGTAGCGGCACTC |
| | | ACTTCACTCTGACCATTAACAGCCTCCAGTCCGAGGATTCAGCCACT |
| | | TACTACTGTCAGCAGTCCTACAAGCGGGCCAGCTTCGGACAGGGCA |
| | | CTAAGGTCGAGATCAAGACCACTACCCCAGCACCGAGGCCACCCAC |
| | | CCCGGCTCCTACCATCGCCTCCCAGCCTCTGTCCCTGCGTCCGGAGG |
| | | CATGTAGACCCGCAGCTGGTGGGGCCGTGCATACCCGGGGTCTTGA |
| | | CTTCGCCTGCGATATCTACATTTGGGCCCCTCTGGCTGGTACTTGCG |
| | | GGGTCCTGCTGCTTTCACTCGTGATCACTCTTTACTGTAAGCGCGGT |
| | | CGGAAGAAGCTGCTGTACATCTTTAAGCAACCCTTCATGAGGCCTG |
| | | TGCAGACTACTCAAGAGGAGGACGGCTGTTCATGCCGGTTCCCAGA |
| | | GGAGGAGGAAGGCGGCTGCGAACTGCGCGTGAAATTCAGCCGCAG |
| | | CGCAGATGCTCCAGCCTACAAGCAGGGGCAGAACCAGCTCTACAAC |
| | | GAACTCAATCTTGGTCGGAGAGAGGAGTACGACGTGCTGGACAAG |
| | | CGGAGAGGACGGGACCCAGAAATGGGCGGGAAGCCGCGCAGAAA |
| | | GAATCCCCAAGAGGGCCTGTACAACGAGCTCCAAAAGGATAAGAT |
| | | GGCAGAAGCCTATAGCGAGATTGGTATGAAAGGGGAACGCAGAAG |
| | | AGGCAAAGGCCACGACGGACTGTACCAGGGACTCAGCACCGCCAC |
| | | CAAGGACACCTATGACGCTCTTCACATGCAGGCCCTGCCGCCTCGG |

139102

| | | |
|---|---|---|
| 139102-aa | 44 | QVQLVQSGAEVKKPGASVKVSCKASGYTFSNYGITWVRQAPGQGLE |
| ScFv domain | | WMGWISAYNGNTNYAQKFQGRVTMTRNTSISTAYMELSSLRSEDTAV |
| | | YYCARGPYYYYMDVWGKGTMVTVSSASGGGGSGGRASGGGGSEIV |
| | | MTQSPLSLPVTPGEPASISCRSSQSLLYSNGYNYVDWYLQKPGQSPQLL |
| | | IYLGSNRASGVPDRFSGSGSGTDFKLQISRVEAEDVGIYYCMQGRQFPY |
| | | SFGQGTKVEIK |
| 139102-nt | 59 | CAAGTCCAACTGGTCCAGAGCGGTGCAGAAGTGAAGAAGCCCGGA |
| ScFv domain | | GCGAGCGTGAAAGTGTCCTGCAAGGCTTCCGGGTACACCTTCTCCA |
| | | ACTACGGCATCACTTGGGTGCGCCAGGCCCCGGGACAGGGCCTGGA |
| | | ATGGATGGGGTGGATTTCCGCGTACAACGGCAATACGAACTACGCT |
| | | CAGAAGTTCCAGGGTAGAGTGACCATGACTAGGAACACCTCCATTT |
| | | CCACCGCCTACATGGAACTGTCCTCCCTGCGGAGCGAGGACACCGC |
| | | CGTGTACTATTGCGCCCGGGGACCATACTACTACTACATGGATGTCT |
| | | GGGGGAAGGGGACTATGGTCACCGTGTCATCCGCCTCGGGAGGCG |
| | | GCGGATCAGGAGGACGCGCCTCTGGTGGTGGAGGATCGGAGATCG |
| | | TGATGACCCAGAGCCCTCTCTCCTTGCCCGTGACTCCTGGGGAGCCC |
| | | GCATCCATTTCATGCCGGAGCTCCCAGTCACTTCTCTACTCCAACGG |
| | | CTATAACTACGTGGATTGGTACCTCCAAAAGCCGGGCCAGAGCCCG |
| | | CAGCTGCTGATCTACCTGGGCTCGAACAGGGCCAGCGGAGTGCCTG |
| | | ACCGGTTCTCCGGGTCGGGAAGCGGGACCGACTTCAAGCTGCAAAT |
| | | CTCGAGAGTGGAGGCCGAGGACGTGGGAATCTACTACTGTATGCAG |
| | | GGCCGCCAGTTTCCGTACTCGTTCGGACAGGGCACCAAAGTGGAAA |
| | | TCAAG |
| 139102-aa | 74 | QVQLVQSGAEVKKPGASVKVSCKASGYTFSNYGITWVRQAPGQGLE |
| VH | | WMGWISAYNGNTNYAQKFQGRVTMTRNTSISTAYMELSSLRSEDTAV |
| | | YYCARGPYYYYMDVWGKGTMVTVSS |
| 139102-aa | 89 | EIVMTQSPLSLPVTPGEPASISCRSSQSLLYSNGYNYVDWYLQKPGQSP |
| VL | | QLLIYLGSNRASGVPDRFSGSGSGTDFKLQISRVEAEDVGIYYCMQGR |
| | | QFPYSFGQGTKVEIK |
| 139102-aa | 104 | MALPVTALLLPLALLLHAARPQVQLVQSGAEVKKPGASVKVSCKASG |
| Full CAR | | YTFSNYGITWVRQAPGQGLEWMGWISAYNGNTNYAQKFQGRVTMTR |
| | | NTSISTAYMELSSLRSEDTAVYYCARGPYYYYMDVWGKGTMVTVSSA |
| | | SGGGGSGGRASGGGGSEIVMTQSPLSLPVTPGEPASISCRSSQSLLYSNG |
| | | YNYVDWYLQKPGQSPQLLIYLGSNRASGVPDRFSGSGSGTDFKLQISR |
| | | VEAEDVGIYYCMQGRQFPYSFGQGTKVEIKTTTPAPRPPTPAPTIASQP |
| | | LSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRK |
| | | NPQEGLYNELQKDKMAEAYSEIGMKGERRRGKGHDGLYQGLSTATK |
| | | DTYDALHMQALPPR |
| 139102-nt | 119 | ATGGCCCTCCCTGTCACCGCCCTGCTGCTTCCGCTGGCTCTTCTGCT |
| Full CAR | | CCACGCCGCTCGGCCCCAAGTCCAACTGGTCCAGAGCGGTGCAGAA |
| | | GTGAAGAAGCCCGGAGCGAGCGTGAAAGTGTCCTGCAAGGCTTCC |
| | | GGGTACACCTTCTCCAACTACGGCATCACTTGGGTGCGCCAGGCCC |
| | | CGGGACAGGGCCTGGAATGGATGGGGTGGATTTCCGCGTACAACG |
| | | GCAATACGAACTACGCTCAGAAGTTCCAGGGTAGAGTGACCATGAC |
| | | TAGGAACACCTCCATTTCCACCGCCTACATGGAACTGTCCTCCCTGC |
| | | GGAGCGAGGACACCGCCGTGTACTATTGCGCCCGGGGACCATACTA |
| | | CTACTACATGGATGTCTGGGGGAAGGGGACTATGGTCACCGTGTCA |
| | | TCCGCCTCGGGAGGCGGCGGATCAGGAGGACGCGCCTCTGGTGGTG |
| | | GAGGATCGGAGATCGTGATGACCCAGAGCCCTCTCTCCTTGCCCGT |
| | | GACTCCTGGGGAGCCCGCATCCATTTCATGCCGGAGCTCCCAGTCA |
| | | CTTCTCTACTCCAACGGCTATAACTACGTGGATTGGTACCTCCAAAA |
| | | GCCGGGCCAGAGCCCGCAGCTGCTGATCTACCTGGGCTCGAACAGG |
| | | GCCAGCGGAGTGCCTGACCGGTTCTCCGGGTCGGGAAGCGGGACCG |
| | | ACTTCAAGCTGCAAATCTCGAGAGTGGAGGCCGAGGACGTGGGAA |
| | | TCTACTACTGTATGCAGGGCCGCCAGTTTCCGTACTCGTTCGGACAG |
| | | GGCACCAAAGTGGAAATCAAGACCACTACCCCAGCACCGAGGCCA |
| | | CCCACCCCGGCTCCTACCATCGCCTCCCAGCCTCTGTCCCTGCGTCC |
| | | GGAGGCATGTAGACCCGCAGCTGGTGGGGCCGTGCATACCCGGGGT |
| | | CTTGACTTCGCCTGCGATATCTACATTTGGGCCCCTCTGGCTGGTAC |
| | | TTGCGGGGTCCTGCTGCTTTCACTCGTGATCACTCTTTACTGTAAGC |
| | | GCGGTCGGAAGAAGCTGCTGTACATCTTTAAGCAACCCTTCATGAG |
| | | GCCTGTGCAGACTACTCAAGAGGAGGACGGCTGTTCATGCCGGTTC |
| | | CCAGAGGAGGAGGAAGGCGGCTGCGAACTGCGCGTGAAATTCAGC |
| | | CGCAGCGCAGATGCTCCAGCCTACAAGCAGGGGCAGAACCAGCTCT |
| | | ACAACGAACTCAATCTTGGTCGGAGAGAGGAGTACGACGTGCTGG |
| | | ACAAGCGGAGAGGACGGGACCCAGAAATGGGCGGGAAGCCGCGCA |
| | | GAAAGAATCCCCAAGAGGGCCTGTACAACGAGCTCCAAAAGGATA |
| | | AGATGGCAGAAGCCTATAGCGAGATTGGTATGAAAGGGGAACGCA |
| | | GAAGAGGCAAAGGCCACGACGGACTGTACCAGGGACTCAGCACCG |
| | | CCACCAAGGACACCTATGACGCTCTTCACATGCAGGCCCTGCCGCC |
| | | TCGG |

139104

| | | |
|---|---|---|
| 139104-aa | 45 | EVQLLETGGGLVQPGGSLRLSCAVSGFALSNHGMSWVRRAPGKGLEW |
| ScFv domain | | VSGIVYSGSTYYAASVKGRFTISRDNSRNTLYLQMNSLRPEDTAIYYCS |
| | | AHGGESDVWGQGTTVTVSSASGGGGSGGRASGGGGSEIVLTQSPATLS |
| | | VSPGESATLSCRASQSVSSNLAWYQQKPGQAPRLLIYGASTRASGIPDR |
| | | FSGSGSGTDFTLTISSLQAEDVAVYYCQQYGSSLTFGGGTKVEIK |
| 139104-nt | 60 | GAAGTGCAATTGCTCGAAACTGGAGGAGGTCTGGTGCAACCTGGAG |
| ScFv domain | | GATCACTTCGCCTGTCCTGCGCCGTGTCGGGCTTTGCCCTGTCCAAC |
| | | CATGGAATGAGCTGGGTCCGCCGCGCGCCGGGGAAGGGCCTCGAA |
| | | TGGGTGTCCGGCATCGTCTACTCCGGCTCCACCTACTACGCCGCGTC |
| | | CGTGAAGGGCCGGTTCACGATTTCACGGGACAACTCGCGGAACACC |
| | | CTGTACCTCCAAATGAATTCCCTTCGGCCGGAGGATACTGCCATCTA |
| | | CTACTGCTCCGCCCACGGTGGCGAATCCGACGTCTGGGGCCAGGGA |
| | | ACCACCGTGACCGTGTCCAGCGCGTCCGGGGGAGGAGGAAGCGGG |
| | | GGTAGAGCATCGGGTGGAGGCGGATCAGAGATCGTGCTGACCCAG |
| | | TCCCCCGCCACCTTGAGCGTGTCACCAGGAGAGTCCGCCACCCTGT |
| | | CATGCCGCGCCAGCCAGTCCGTGTCCTCCAACCTGGCTTGGTACCA |
| | | GCAGAAGCCGGGGCAGGCCCCTAGACTCCTGATCTATGGGGCGTCG |
| | | ACCCGGGCATCTGGAATTCCCGATAGGTTCAGCGGATCGGGCTCGG |
| | | GCACTGACTTCACTCTGACCATCTCCTCGCTGCAAGCCGAGGACGT |
| | | GGCTGTGTACTACTGTCAGCAGTACGGAAGCTCCCTGACTTTCGGT |
| | | GGCGGGACCAAAGTCGAGATTAAG |
| 139104-aa | 75 | EVQLLETGGGLVQPGGSLRLSCAVSGFALSNHGMSWVRRAPGKGLEW |
| VH | | VSGIVYSGSTYYAASVKGRFTISRDNSRNTLYLQMNSLRPEDTAIYYCS |
| | | AHGGESDVWGQGTTVTVSS |
| 139104-aa | 90 | EIVLTQSPATLSVSPGESATLSCRASQSVSSNLAWYQQKPGQAPRLLIY |
| VL | | GASTRASGIPDRFSGSGSGTDFTLTISSLQAEDVAVYYCQQYGSSLTFG |
| | | GGTKVEIK |
| 139104-aa | 105 | MALPVTALLLPLALLLHAARPEVQLLETGGGLVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSEIVLTQSPATLSVSPGESATLSCRASQSVSSNLAWYQQ |
| | | KPGQAPRLLIYGASTRASGIPDRFSGSGSGTDFTLTISSLQAEDVAVYYC |
| | | QQYGSSLTFGGGTKVEIKTTTPAPRPPTPAPTIASQPLSLRPEACRPAAG |
| | | GAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQK |
| | | DKMAEAYSEIGMKGERRRGKGHDGLYQGLSTATKDTYDALHMQALP |
| | | PR |
| 139104-nt | 120 | ATGGCCCTCCCTGTCACCGCCCTGCTGCTTCCGCTGGCTCTTCTGCT |
| Full CAR | | CCACGCCGCTCGGCCCGAAGTGCAATTGCTCGAAACTGGAGGAGGT |
| | | CTGGTGCAACCTGGAGGATCACTTCGCCTGTCCTGCGCCGTGTCGG |
| | | GCTTTGCCCTGTCCAACCATGGAATGAGCTGGGTCCGCCGCGCGCC |
| | | GGGGAAGGGCCTCGAATGGGTGTCCGGCATCGTCTACTCCGGCTCC |
| | | ACCTACTACGCCGCGTCCGTGAAGGGCCGGTTCACGATTTCACGGG |
| | | ACAACTCGCGGAACACCCTGTACCTCCAAATGAATTCCCTTCGGCC |
| | | GGAGGATACTGCCATCTACTACTGCTCCGCCCACGGTGGCGAATCC |
| | | GACGTCTGGGGCCAGGGAACCACCGTGACCGTGTCCAGCGCGTCCG |
| | | GGGGAGGAGGAAGCGGGGGTAGAGCATCGGGTGGAGGCGGATCAG |
| | | AGATCGTGCTGACCCAGTCCCCCGCCACCTTGAGCGTGTCACCAGG |
| | | AGAGTCCGCCACCCTGTCATGCCGCGCCAGCCAGTCCGTGTCCTCC |
| | | AACCTGGCTTGGTACCAGCAGAAGCCGGGGCAGGCCCCTAGACTCC |
| | | TGATCTATGGGGCGTCGACCCGGGCATCTGGAATTCCCGATAGGTT |
| | | CAGCGGATCGGGCTCGGGCACTGACTTCACTCTGACCATCTCCTCG |
| | | CTGCAAGCCGAGGACGTGGCTGTGTACTACTGTCAGCAGTACGGAA |
| | | GCTCCCTGACTTTCGGTGGCGGGACCAAAGTCGAGATTAAGACCAC |
| | | TACCCCAGCACCGAGGCCACCCACCCCGGCTCCTACCATCGCCTCC |
| | | CAGCCTCTGTCCCTGCGTCCGGAGGCATGTAGACCCGCAGCTGGTG |
| | | GGGCCGTGCATACCCGGGGTCTTGACTTCGCCTGCGATATCTACATT |
| | | TGGGCCCCTCTGGCTGGTACTTGCGGGGTCCTGCTGCTTTCACTCGT |
| | | GATCACTCTTTACTGTAAGCGCGGTCGGAAGAAGCTGCTGTACATC |
| | | TTTAAGCAACCCTTCATGAGGCCTGTGCAGACTACTCAAGAGGAGG |
| | | ACGGCTGTTCATGCCGGTTCCCAGAGGAGGAGGAAGGCGGCTGCG |
| | | AACTGCGCGTGAAATTCAGCCGCAGCGCAGATGCTCCAGCCTACAA |
| | | GCAGGGGCAGAACCAGCTCTACAACGAACTCAATCTTGGTCGGAGA |
| | | GAGGAGTACGACGTGCTGGACAAGCGGAGAGGACGGGACCCAGAA |
| | | ATGGGCGGGAAGCCGCGCAGAAAGAATCCCCAAGAGGGCCTGTAC |
| | | AACGAGCTCCAAAAGGATAAGATGGCAGAAGCCTATAGCGAGATT |
| | | GGTATGAAAGGGGAACGCAGAAGAGGCAAAGGCCACGACGGACTG |
| | | TACCAGGGACTCAGCACCGCCACCAAGGACACCTATGACGCTCTTC |
| | | ACATGCAGGCCCTGCCGCCTCGG |

139106

| | | |
|---|---|---|
| 139106-aa | 46 | EVQLVETGGGLVQPGGSLRLSCAVSGFALSNHGMSWVRRAPGKGLE |
| ScFv domain | | WVSGIVYSGSTYYAASVKGRFTISRDNSRNTLYLQMNSLRPEDTAIYY |
| | | CSAHGGESDVWGQGTTVTVSSASGGGGSGGRASGGGGSEIVMTQSPA |
| | | TLSVSPGERATLSCRASQSVSSKLAWYQQKPGQAPRLLMYGASIRATG |
| | | IPDRFSGSGSGTEFTLTISSLEPEDFAVYYCQQYGSSSWTFGQGTKVEIK |
| 139106-nt | 61 | GAAGTGCAATTGGTGGAAACTGGAGGAGGACTTGTGCAACCTGGA |
| ScFv domain | | GGATCATTGAGACTGAGCTGCGCAGTGTCGGGATTCGCCCTGAGCA |
| | | ACCATGGAATGTCCTGGGTCAGAAGGGCCCCTGGAAAAGGCCTCGA |
| | | ATGGGTGTCAGGGATCGTGTACTCCGGTTCCACTTACTACGCCGCCT |
| | | CCGTGAAGGGGCGCTTCACTATCTCACGGGATAACTCCCGCAATAC |
| | | CCTGTACCTCCAAATGAACAGCCTGCGGCCGGAGGATACCGCCATC |
| | | TACTACTGTTCCGCCCACGGTGGAGAGTCTGACGTCTGGGGCCAGG |
| | | GAACTACCGTGACCGTGTCCTCCGCGTCCGGCGGTGGAGGGAGCGG |
| | | CGGCCGCGCCAGCGGCGGCGGAGGCTCCGAGATCGTGATGACCCA |
| | | GAGCCCCGCTACTCTGTCGGTGTCGCCCGGAGAAAGGGCGACCCTG |
| | | TCCTGCCGGGCGTCGCAGTCCGTGAGCAGCAAGCTGGCTTGGTACC |
| | | AGCAGAAGCCGGGCCAGGCACCACGCCTGCTTATGTACGGTGCCTC |
| | | CATTCGGGCCACCGGAATCCCGGACCGGTTCTCGGGGTCGGGGTCC |
| | | GGTACCGAGTTCACACTGACCATTTCCTCGCTCGAGCCCGAGGACT |
| | | TTGCCGTCTATTACTGCCAGCAGTACGGCTCCTCCTCATGGACGTTC |
| | | GGCCAGGGGACCAAGGTCGAAATCAAG |
| 139106-aa | 76 | EVQLVETGGGLVQPGGSLRLSCAVSGFALSNHGMSWVRRAPGKGLE |
| VH | | WVSGIVYSGSTYYAASVKGRFTISRDNSRNTLYLQMNSLRPEDTAIYY |
| | | CSAHGGESDVWGQGTTVTVSS |
| 139106-aa | 91 | EIVMTQSPATLSVSPGERATLSCRASQSVSSKLAWYQQKPGQAPRLLM |
| VL | | YGASIRATGIPDRFSGSGSGTEFTLTISSLEPEDFAVYYCQQYGSSSWTF |
| | | GQGTKVEIK |
| 139106-aa | 106 | MALPVTALLLPLALLLHAARPEVQLVETGGGLVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSEIVMTQSPATLSVSPGERATLSCRASQSVSSKLAWYQ |
| | | QKPGQAPRLLMYGASIRATGIPDRFSGSGSGTEFTLTISSLEPEDFAVYY |
| | | CQQYGSSSWTFGQGTKVEIKTTTPAPRPPTPAPTIASQPLSLRPEACRPA |
| | | AGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQ |
| | | ALPPR |
| 139106-nt | 121 | ATGGCCCTCCCTGTCACCGCCCTGCTGCTTCCGCTGGCTCTTCTGCT |
| Full CAR | | CCACGCCGCTCGGCCCGAAGTGCAATTGGTGGAAACTGGAGGAGG |
| | | ACTTGTGCAACCTGGAGGATCATTGAGACTGAGCTGCGCAGTGTCG |
| | | GGATTCGCCCTGAGCAACCATGGAATGTCCTGGGTCAGAAGGGCCC |
| | | CTGGAAAAGGCCTCGAATGGGTGTCAGGGATCGTGTACTCCGGTTC |
| | | CACTTACTACGCCGCCTCCGTGAAGGGGCGCTTCACTATCTCACGG |
| | | GATAACTCCCGCAATACCCTGTACCTCCAAATGAACAGCCTGCGGC |
| | | CGGAGGATACCGCCATCTACTACTGTTCCGCCCACGGTGGAGAGTC |
| | | TGACGTCTGGGGCCAGGGAACTACCGTGACCGTGTCCTCCGCGTCC |
| | | GGCGGTGGAGGGAGCGGCGGCCGCGCCAGCGGCGGCGGAGGCTCC |
| | | GAGATCGTGATGACCCAGAGCCCCGCTACTCTGTCGGTGTCGCCCG |
| | | GAGAAAGGGCGACCCTGTCCTGCCGGGCGTCGCAGTCCGTGAGCAG |
| | | CAAGCTGGCTTGGTACCAGCAGAAGCCGGGCCAGGCACCACGCCTG |
| | | CTTATGTACGGTGCCTCCATTCGGGCCACCGGAATCCCGGACCGGT |
| | | TCTCGGGGTCGGGGTCCGGTACCGAGTTCACACTGACCATTTCCTCG |
| | | CTCGAGCCCGAGGACTTTGCCGTCTATTACTGCCAGCAGTACGGCT |
| | | CCTCCTCATGGACGTTCGGCCAGGGGACCAAGGTCGAAATCAAGAC |
| | | CACTACCCCAGCACCGAGGCCACCCACCCCGGCTCCTACCATCGCC |
| | | TCCCAGCCTCTGTCCCTGCGTCCGGAGGCATGTAGACCCGCAGCTG |
| | | GTGGGGCCGTGCATACCCGGGGTCTTGACTTCGCCTGCGATATCTA |
| | | CATTTGGGCCCCTCTGGCTGGTACTTGCGGGGTCCTGCTGCTTTCAC |
| | | TCGTGATCACTCTTTACTGTAAGCGCGGTCGGAAGAAGCTGCTGTA |
| | | CATCTTTAAGCAACCCTTCATGAGGCCTGTGCAGACTACTCAAGAG |
| | | GAGGACGGCTGTTCATGCCGGTTCCCAGAGGAGGAGGAAGGCGGC |
| | | TGCGAACTGCGCGTGAAATTCAGCCGCAGCGCAGATGCTCCAGCCT |
| | | ACAAGCAGGGGCAGAACCAGCTCTACAACGAACTCAATCTTGGTCG |
| | | GAGAGAGGAGTACGACGTGCTGGACAAGCGGAGAGGACGGGACCC |
| | | AGAAATGGGCGGGAAGCCGCGCAGAAAGAATCCCCAAGAGGGCCT |
| | | GTACAACGAGCTCCAAAAGGATAAGATGGCAGAAGCCTATAGCGA |
| | | GATTGGTATGAAAGGGGAACGCAGAAGAGGCAAAGGCCACGACGG |
| | | ACTGTACCAGGGACTCAGCACCGCCACCAAGGACACCTATGACGCT |
| | | CTTCACATGCAGGCCCTGCCGCCTCGG |

139107

| | | |
|---|---|---|
| 139107-aa | 47 | EVQLVETGGGVVQPGGSLRLSCAVSGFALSNHGMSWVRRAPGKGLE |
| ScFv domain | | WVSGIVYSGSTYYAASVKGRFTISRDNSRNTLYLQMNSLRPEDTAIYY |
| | | CSAHGGESDVWGQGTTVTVSSASGGGGSGGRASGGGGSEIVLTQSPG |
| | | TLSLSPGERATLSCRASQSVGSTNLAWYQQKPGQAPRLLIYDASNRAT |
| | | GIPDRFSGGGSGTDFTLTISRLEPEDFAVYYCQQYGSSPPWTFGQGTKV |
| | | EIK |
| 139107-nt | 62 | GAAGTGCAATTGGTGGAGACTGGAGGAGGAGTGGTGCAACCTGGA |
| ScFv domain | | GGAAGCCTGAGACTGTCATGCGCGGTGTCGGGCTTCGCCCTCTCCA |
| | | ACCACGGAATGTCCTGGGTCCGCCGGGCCCCTGGGAAAGGACTTGA |
| | | ATGGGTGTCCGGCATCGTGTACTCGGGTTCCACCTACTACGCGGCCT |
| | | CAGTGAAGGGCCGGTTTACTATTAGCCGCGACAACTCCAGAAACAC |
| | | ACTGTACCTCCAAATGAACTCGCTGCGGCCGGAAGATACCGCTATC |
| | | TACTACTGCTCCGCCCATGGGGGAGAGTCGGACGTCTGGGGACAGG |
| | | GCACCACTGTCACTGTGTCCAGCGCTTCCGGCGGTGGTGGAAGCGG |
| | | GGGACGGGCCTCAGGAGGCGGTGGCAGCGAGATTGTGCTGACCCA |
| | | GTCCCCCGGGACCCTGAGCCTGTCCCCGGGAGAAAGGGCCACCCTC |
| | | TCCTGTCGGGCATCCCAGTCCGTGGGGTCTACTAACCTTGCATGGTA |
| | | CCAGCAGAAGCCCGGCCAGGCCCCTCGCCTGCTGATCTACGACGCG |
| | | TCCAATAGAGCCACCGGCATCCCGGATCGCTTCAGCGGAGGCGGAT |
| | | CGGGCACCGACTTCACCCTCACCATTTCAAGGCTGGAACCGGAGGA |
| | | CTTCGCCGTGTACTACTGCCAGCAGTATGGTTCGTCCCCACCCTGGA |
| | | CGTTCGGCCAGGGGACTAAGGTCGAGATCAAG |
| 139107-aa | 77 | EVQLVETGGGVVQPGGSLRLSCAVSGFALSNHGMSWVRRAPGKGLE |
| VH | | WVSGIVYSGSTYYAASVKGRFTISRDNSRNTLYLQMNSLRPEDTAIYY |
| | | CSAHGGESDVWGQGTTVTVSS |
| 139107-aa | 92 | EIVLTQSPGTLSLSPGERATLSCRASQSVGSTNLAWYQQKPGQAPRLLI |
| VL | | YDASNRATGIPDRFSGGGSGTDFTLTISRLEPEDFAVYYCQQYGSSPPW |
| | | TFGQGTKVEIK |
| 139107-aa | 107 | MALPVTALLLPLALLLHAARPEVQLVETGGGVVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSEIVLTQSPGTLSLSPGERATLSCRASQSVGSTNLAWYQ |
| | | QKPGQAPRLLIYDASNRATGIPDRFSGGGSGTDFTLTISRLEPEDFAVYY |
| | | CQQYGSSPPWTFGQGTKVEIKTTTPAPRPPTPAPTIASQPLSLRPEACRP |
| | | AAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 139107-nt | 122 | ATGGCCCTCCCTGTCACCGCCCTGCTGCTTCCGCTGGCTCTTCTGCT |
| Full CAR | | CCACGCCGCTCGGCCCGAAGTGCAATTGGTGGAGACTGGAGGAGG |
| | | AGTGGTGCAACCTGGAGGAAGCCTGAGACTGTCATGCGCGGTGTCG |
| | | GGCTTCGCCCTCTCCAACCACGGAATGTCCTGGGTCCGCCGGGCCC |
| | | CTGGGAAAGGACTTGAATGGGTGTCCGGCATCGTGTACTCGGGTTC |
| | | CACCTACTACGCGGCCTCAGTGAAGGGCCGGTTTACTATTAGCCGC |
| | | GACAACTCCAGAAACACACTGTACCTCCAAATGAACTCGCTGCGGC |
| | | CGGAAGATACCGCTATCTACTACTGCTCCGCCCATGGGGGAGAGTC |
| | | GGACGTCTGGGGACAGGGCACCACTGTCACTGTGTCCAGCGCTTCC |
| | | GGCGGTGGTGGAAGCGGGGGACGGGCCTCAGGAGGCGGTGGCAGC |
| | | GAGATTGTGCTGACCCAGTCCCCCGGGACCCTGAGCCTGTCCCCGG |
| | | GAGAAAGGGCCACCCTCTCCTGTCGGGCATCCCAGTCCGTGGGGTC |
| | | TACTAACCTTGCATGGTACCAGCAGAAGCCCGGCCAGGCCCCTCGC |
| | | CTGCTGATCTACGACGCGTCCAATAGAGCCACCGGCATCCCGGATC |
| | | GCTTCAGCGGAGGCGGATCGGGCACCGACTTCACCCTCACCATTTC |
| | | AAGGCTGGAACCGGAGGACTTCGCCGTGTACTACTGCCAGCAGTAT |
| | | GGTTCGTCCCCACCCTGGACGTTCGGCCAGGGGACTAAGGTCGAGA |
| | | TCAAGACCACTACCCCAGCACCGAGGCCACCCACCCCGGCTCCTAC |
| | | CATCGCCTCCCAGCCTCTGTCCCTGCGTCCGGAGGCATGTAGACCC |
| | | GCAGCTGGTGGGGCCGTGCATACCCGGGGTCTTGACTTCGCCTGCG |
| | | ATATCTACATTTGGGCCCCTCTGGCTGGTACTTGCGGGGTCCTGCTG |
| | | CTTTCACTCGTGATCACTCTTTACTGTAAGCGCGGTCGGAAGAAGCT |
| | | GCTGTACATCTTTAAGCAACCCTTCATGAGGCCTGTGCAGACTACTC |
| | | AAGAGGAGGACGGCTGTTCATGCCGGTTCCCAGAGGAGGAGGAAG |
| | | GCGGCTGCGAACTGCGCGTGAAATTCAGCCGCAGCGCAGATGCTCC |
| | | AGCCTACAAGCAGGGGCAGAACCAGCTCTACAACGAACTCAATCTT |
| | | GGTCGGAGAGAGGAGTACGACGTGCTGGACAAGCGGAGAGGACGG |
| | | GACCCAGAAATGGGCGGGAAGCCGCGCAGAAAGAATCCCCAAGAG |
| | | GGCCTGTACAACGAGCTCCAAAAGGATAAGATGGCAGAAGCCTAT |
| | | AGCGAGATTGGTATGAAAGGGGAACGCAGAAGAGGCAAAGGCCAC |
| | | GACGGACTGTACCAGGGACTCAGCACCGCCACCAAGGACACCTATG |
| | | ACGCTCTTCACATGCAGGCCCTGCCGCCTCGG |

139108

| | | |
|---|---|---|
| 139108-aa | 48 | QVQLVESGGGLVKPGGSLRLSCAASGFTFSDYYMSWIRQAPGKGLEW |
| ScFv domain | | VSYISSSGSTIYYADSVKGRFTISRDNAKNSLYLQMNSLRAEDTAVYYC |
| | | ARESGDGMDVWGQGTTVTVSSASGGGGSGGRASGGGGSDIQMTQSPS |
| | | SLSASVGDRVTITCRASQSISSYLNWYQQKPGKAPKLLIYAASSLQSGV |
| | | PSRFSGSGSGTDFTLTISSLQPEDFATYYCQQSYTLAFGQGTKVDIK |
| 139108-nt | 63 | CAAGTGCAACTCGTGGAATCTGGTGGAGGACTCGTGAAACCTGGAG |
| ScFv domain | | GATCATTGAGACTGTCATGCGCGGCCTCGGGATTCACGTTCTCCGAT |
| | | TACTACATGAGCTGGATTCGCCAGGCTCCGGGGAAGGGACTGGAAT |
| | | GGGTGTCCTACATTTCCTCATCCGGCTCCACCATCTACTACGCGGAC |
| | | TCCGTGAAGGGGAGATTCACCATTAGCCGCGATAACGCCAAGAACA |
| | | GCCTGTACCTTCAGATGAACTCCCTGCGGGCTGAAGATACTGCCGT |
| | | CTACTACTGCGCAAGGGAGAGCGGAGATGGGATGGACGTCTGGGG |
| | | ACAGGGTACCACTGTGACCGTGTCGTCGGCCTCCGGCGGAGGGGGT |
| | | TCGGGTGGAAGGGCCAGCGGCGGCGGAGGCAGCGACATCCAGATG |
| | | ACCCAGTCCCCCTCATCGCTGTCCGCCTCCGTGGGCGACCGCGTCAC |
| | | CATCACATGCCGGGCCTCACAGTCGATCTCCTCCTACCTCAATTGGT |
| | | ATCAGCAGAAGCCCGGAAAGGCCCCTAAGCTTCTGATCTACGCAGC |
| | | GTCCTCCCTGCAATCCGGGGTCCCATCTCGGTTCTCCGGCTCGGGCA |
| | | GCGGTACCGACTTCACTCTGACCATCTCGAGCCTGCAGCCGGAGGA |
| | | CTTCGCCACTTACTACTGTCAGCAAAGCTACACCCTCGCGTTTGGCC |
| | | AGGGCACCAAAGTGGACATCAAG |
| 139108-aa | 78 | QVQLVESGGGLVKPGGSLRLSCAASGFTFSDYYMSWIRQAPGKGLEW |
| VH | | VSYISSSGSTIYYADSVKGRFTISRDNAKNSLYLQMNSLRAEDTAVYYC |
| | | ARESGDGMDVWGQGTTVTVSS |
| 139108-aa | 93 | DIQMTQSPSSLSASVGDRVTITCRASQSISSYLNWYQQKPGKAPKLLIY |
| VL | | AASSLQSGVPSRFSGSGSGTDFTLTISSLQPEDFATYYCQQSYTLAFGQ |
| | | GTKVDIK |
| 139108-aa | 108 | MALPVTALLLPLALLLHAARPQVQLVESGGGLVKPGGSLRLSCAASGF |
| Full CAR | | TFSDYYMSWIRQAPGKGLEWVSYISSSGSTIYYADSVKGRFTISRDNAK |
| | | NSLYLQMNSLRAEDTAVYYCARESGDGMDVWGQGTTVTVSSASGGG |
| | | GSGGRASGGGGSDIQMTQSPSSLSASVGDRVTITCRASQSISSYLNWYQ |
| | | QKPGKAPKLLIYAASSLQSGVPSRFSGSGSGTDFTLTISSLQPEDFATYY |
| | | CQQSYTLAFGQGTKVDIKTTTPAPRPPTPAPTIASQPLSLRPEACRPAAG |
| | | GAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQK |
| | | DKMAEAYSEIGMKGERRRGKGHDGLYQGLSTATKDTYDALHMQALP |
| | | PR |
| 139108-nt | 123 | ATGGCCCTCCCTGTCACCGCCCTGCTGCTTCCGCTGGCTCTTCTGCT |
| Full CAR | | CCACGCCGCTCGGCCCCAAGTGCAACTCGTGGAATCTGGTGGAGGA |
| | | CTCGTGAAACCTGGAGGATCATTGAGACTGTCATGCGCGGCCTCGG |
| | | GATTCACGTTCTCCGATTACTACATGAGCTGGATTCGCCAGGCTCCG |
| | | GGGAAGGGACTGGAATGGGTGTCCTACATTTCCTCATCCGGCTCCA |
| | | CCATCTACTACGCGGACTCCGTGAAGGGGAGATTCACCATTAGCCG |
| | | CGATAACGCCAAGAACAGCCTGTACCTTCAGATGAACTCCCTGCGG |
| | | GCTGAAGATACTGCCGTCTACTACTGCGCAAGGGAGAGCGGAGATG |
| | | GGATGGACGTCTGGGGACAGGGTACCACTGTGACCGTGTCGTCGGC |
| | | CTCCGGCGGAGGGGGTTCGGGTGGAAGGGCCAGCGGCGGCGGAGG |
| | | CAGCGACATCCAGATGACCCAGTCCCCCTCATCGCTGTCCGCCTCC |
| | | GTGGGCGACCGCGTCACCATCACATGCCGGGCCTCACAGTCGATCT |
| | | CCTCCTACCTCAATTGGTATCAGCAGAAGCCCGGAAAGGCCCCTAA |
| | | GCTTCTGATCTACGCAGCGTCCTCCCTGCAATCCGGGGTCCCATCTC |
| | | GGTTCTCCGGCTCGGGCAGCGGTACCGACTTCACTCTGACCATCTCG |
| | | AGCCTGCAGCCGGAGGACTTCGCCACTTACTACTGTCAGCAAAGCT |
| | | ACACCCTCGCGTTTGGCCAGGGCACCAAAGTGGACATCAAGACCAC |
| | | TACCCCAGCACCGAGGCCACCCACCCCGGCTCCTACCATCGCCTCC |
| | | CAGCCTCTGTCCCTGCGTCCGGAGGCATGTAGACCCGCAGCTGGTG |
| | | GGGCCGTGCATACCCGGGGTCTTGACTTCGCCTGCGATATCTACATT |
| | | TGGGCCCCTCTGGCTGGTACTTGCGGGGTCCTGCTGCTTTCACTCGT |
| | | GATCACTCTTTACTGTAAGCGCGGTCGGAAGAAGCTGCTGTACATC |
| | | TTTAAGCAACCCTTCATGAGGCCTGTGCAGACTACTCAAGAGGAGG |
| | | ACGGCTGTTCATGCCGGTTCCCAGAGGAGGAGGAAGGCGGCTGCG |
| | | AACTGCGCGTGAAATTCAGCCGCAGCGCAGATGCTCCAGCCTACAA |
| | | GCAGGGGCAGAACCAGCTCTACAACGAACTCAATCTTGGTCGGAGA |
| | | GAGGAGTACGACGTGCTGGACAAGCGGAGAGGACGGGACCCAGAA |
| | | ATGGGCGGGAAGCCGCGCAGAAAGAATCCCCAAGAGGGCCTGTAC |
| | | AACGAGCTCCAAAAGGATAAGATGGCAGAAGCCTATAGCGAGATT |
| | | GGTATGAAAGGGGAACGCAGAAGAGGCAAAGGCCACGACGGACTG |
| | | TACCAGGGACTCAGCACCGCCACCAAGGACACCTATGACGCTCTTC |
| | | ACATGCAGGCCCTGCCGCCTCGG |

139110

| | | |
|---|---|---|
| 139110-aa | 50 | QVQLVQSGGGLVKPGGSLRLSCAASGFTFSDYYMSWIRQAPGKGLEW |
| ScFv domain | | VSYISSSGNTIYYADSVKGRFTISRDNAKNSLYLQMNSLRAEDTAVYY |
| | | CARSTMVREDYWGQGTLVTVSSASGGGGSGGRASGGGGSDIVLTQSP |
| | | LSLPVTLGQPASISCKSSESLVHNSGKTYLNWFHQRPGQSPRRLIYEVS |
| | | NRDSGVPDRFTGSGSGTDFTLKISRVEAEDVGVYYCMQGTHWPGTFG |
| | | QGTKLEIK |
| 139110-nt | 65 | CAAGTGCAACTGGTGCAAAGCGGAGGAGGATTGGTCAAACCCGGA |
| ScFv domain | | GGAAGCCTGAGACTGTCATGCGCGGCCTCTGGATTCACCTTCTCCG |
| | | ATTACTACATGTCATGGATCAGACAGGCCCCGGGGAAGGGCCTCGA |
| | | ATGGGTGTCCTACATCTCGTCCTCCGGGAACACCATCTACTACGCCG |
| | | ACAGCGTGAAGGGCCGCTTTACCATTTCCCGCGACAACGCAAAGAA |
| | | CTCGCTGTACCTTCAGATGAATTCCCTGCGGGCTGAAGATACCGCG |
| | | GTGTACTATTGCGCCCGGTCCACTATGGTCCGGGAGGACTACTGGG |
| | | GACAGGGCACACTCGTGACCGTGTCCAGCGCGAGCGGGGGTGGAG |
| | | GCAGCGGTGGACGCGCCTCCGGCGGCGGCGGTTCAGACATCGTGCT |
| | | GACTCAGTCGCCCCTGTCGCTGCCGGTCACCCTGGGCCAACCGGCC |
| | | TCAATTAGCTGCAAGTCCTCGGAGAGCCTGGTGCACAACTCAGGAA |
| | | AGACTTACCTGAACTGGTTCCATCAGCGGCCTGGACAGTCCCCACG |
| | | GAGGCTCATCTATGAAGTGTCCAACAGGGATTCGGGGGTGCCCGAC |
| | | CGCTTCACTGGCTCCGGGTCCGGCACCGACTTCACCTTGAAAATCTC |
| | | CAGAGTGGAAGCCGAGGACGTGGGCGTGTACTACTGTATGCAGGGT |
| | | ACCCACTGGCCTGGAACCTTTGGACAAGGAACTAAGCTCGAGATTA |
| | | AG |
| 139110-aa | 80 | QVQLVQSGGGLVKPGGSLRLSCAASGFTFSDYYMSWIRQAPGKGLEW |
| VH | | VSYISSSGNTIYYADSVKGRFTISRDNAKNSLYLQMNSLRAEDTAVYY |
| | | CARSTMVREDYWGQGTLVTVSS |
| 139110-aa | 95 | DIVLTQSPLSLPVTLGQPASISCKSSESLVHNSGKTYLNWFHQRPGQSPR |
| VL | | RLIYEVSNRDSGVPDRFTGSGSGTDFTLKISRVEAEDVGVYYCMQGTH |
| | | WPGTFGQGTKLEIK |
| 139110-aa | 110 | MALPVTALLLPLALLLHAARPQVQLVQSGGGLVKPGGSLRLSCAASGF |
| Full CAR | | TFSDYYMSWIRQAPGKGLEWVSYISSSGNTIYYADSVKGRFTISRDNA |
| | | KNSLYLQMNSLRAEDTAVYYCARSTMVREDYWGQGTLVTVSSASGG |
| | | GGSGGRASGGGGSDIVLTQSPLSLPVTLGQPASISCKSSESLVHNSGKT |
| | | YLNWFHQRPGQSPRRLIYEVSNRDSGVPDRFTGSGSGTDFTLKISRVEA |
| | | EDVGVYYCMQGTHWPGTFGQGTKLEIKTTTPAPRPPTPAPTIASQPLSL |
| | | RPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDT |
| | | YDALHMQALPPR |
| 139110-nt | 125 | ATGGCCCTCCCTGTCACCGCCCTGCTGCTTCCGCTGGCTCTTCTGCT |
| Full CAR | | CCACGCCGCTCGGCCCCAAGTGCAACTGGTGCAAAGCGGAGGAGG |
| | | ATTGGTCAAACCCGGAGGAAGCCTGAGACTGTCATGCGCGGCCTCT |
| | | GGATTCACCTTCTCCGATTACTACATGTCATGGATCAGACAGGCCCC |
| | | GGGGAAGGGCCTCGAATGGGTGTCCTACATCTCGTCCTCCGGGAAC |
| | | ACCATCTACTACGCCGACAGCGTGAAGGGCCGCTTTACCATTTCCC |
| | | GCGACAACGCAAAGAACTCGCTGTACCTTCAGATGAATTCCCTGCG |
| | | GGCTGAAGATACCGCGGTGTACTATTGCGCCCGGTCCACTATGGTC |
| | | CGGGAGGACTACTGGGGACAGGGCACACTCGTGACCGTGTCCAGC |
| | | GCGAGCGGGGGTGGAGGCAGCGGTGGACGCGCCTCCGGCGGCGGC |
| | | GGTTCAGACATCGTGCTGACTCAGTCGCCCCTGTCGCTGCCGGTCAC |
| | | CCTGGGCCAACCGGCCTCAATTAGCTGCAAGTCCTCGGAGAGCCTG |
| | | GTGCACAACTCAGGAAAGACTTACCTGAACTGGTTCCATCAGCGGC |
| | | CTGGACAGTCCCCACGGAGGCTCATCTATGAAGTGTCCAACAGGGA |
| | | TTCGGGGGTGCCCGACCGCTTCACTGGCTCCGGGTCCGGCACCGAC |
| | | TTCACCTTGAAAATCTCCAGAGTGGAAGCCGAGGACGTGGGCGTGT |
| | | ACTACTGTATGCAGGGTACCCACTGGCCTGGAACCTTTGGACAAGG |
| | | AACTAAGCTCGAGATTAAGACCACTACCCCAGCACCGAGGCCACCC |
| | | ACCCCGGCTCCTACCATCGCCTCCCAGCCTCTGTCCCTGCGTCCGGA |
| | | GGCATGTAGACCCGCAGCTGGTGGGGCCGTGCATACCCGGGGTCTT |
| | | GACTTCGCCTGCGATATCTACATTTGGGCCCCTCTGGCTGGTACTTG |
| | | CGGGGTCCTGCTGCTTTCACTCGTGATCACTCTTTACTGTAAGCGCG |
| | | GTCGGAAGAAGCTGCTGTACATCTTTAAGCAACCCTTCATGAGGCC |
| | | TGTGCAGACTACTCAAGAGGAGGACGGCTGTTCATGCCGGTTCCCA |
| | | GAGGAGGAGGAAGGCGGCTGCGAACTGCGCGTGAAATTCAGCCGC |
| | | AGCGCAGATGCTCCAGCCTACAAGCAGGGGCAGAACCAGCTCTAC |
| | | AACGAACTCAATCTTGGTCGGAGAGAGGAGTACGACGTGCTGGAC |
| | | AAGCGGAGAGGACGGGACCCAGAAATGGGCGGGAAGCCGCGCAG |
| | | AAAGAATCCCCAAGAGGGCCTGTACAACGAGCTCCAAAAGGATAA |
| | | GATGGCAGAAGCCTATAGCGAGATTGGTATGAAAGGGGAACGCAG |
| | | AAGAGGCAAAGGCCACGACGGACTGTACCAGGGACTCAGCACCGC |
| | | CACCAAGGACACCTATGACGCTCTTCACATGCAGGCCCTGCCGCCT |
| | | CGG |

139112

| | | |
|---|---|---|
| 139112-aa | 51 | QVQLVESGGGLVQPGGSLRLSCAVSGFALSNHGMSWVRRAPGKGLE |
| ScFv domain | | WVSGIVYSGSTYYAASVKGRFTISRDNSRNTLYLQMNSLRPEDTAIYY |
| | | CSAHGGESDVWGQGTTVTVSSASGGGGSGGRASGGGGSDIRLTQSPSP |
| | | LSASVGDRVTITCQASEDINKFLNWYHQTPGKAPKLLIYDASTLQTGVP |
| | | SRFSGSGSGTDFTLTINSLQPEDIGTYYCQQYESLPLTFGGGTKVEIK |
| 139112-nt | 66 | CAAGTGCAACTCGTGGAATCTGGTGGAGGACTCGTGCAACCCGGTG |
| ScFv domain | | GAAGCCTTAGGCTGTCGTGCGCCGTCAGCGGGTTTGCTCTGAGCAA |
| | | CCATGGAATGTCCTGGGTCCGCCGGGCACCGGGAAAAGGGCTGGA |
| | | ATGGGTGTCCGGCATCGTGTACAGCGGGTCAACCTATTACGCCGCG |
| | | TCCGTGAAGGGCAGATTCACTATCTCAAGAGACAACAGCCGGAACA |
| | | CCCTGTACTTGCAAATGAATTCCCTGCGCCCCGAGGACACCGCCAT |
| | | CTACTACTGCTCCGCCCACGGAGGAGAGTCGGACGTGTGGGGCCAG |
| | | GGAACGACTGTGACTGTGTCCAGCGCATCAGGAGGGGGTGGTTCGG |
| | | GCGGCCGGGCCTCGGGGGGAGGAGGTTCCGACATTCGGCTGACCCA |
| | | GTCCCCGTCCCCACTGTCGGCCTCCGTCGGCGACCGCGTGACCATC |
| | | ACTTGTCAGGCGTCCGAGGACATTAACAAGTTCCTGAACTGGTACC |
| | | ACCAGACCCCTGGAAAGGCCCCCAAGCTGCTGATCTACGATGCCTC |
| | | GACCCTTCAAACTGGAGTGCCTAGCCGGTTCTCCGGGTCCGGCTCC |
| | | GGCACTGATTTCACTCTGACCATCAACTCATTGCAGCCGGAAGATA |
| | | TCGGGACCTACTATTGCCAGCAGTACGAATCCCTCCCGCTCACATTC |
| | | GGCGGGGGAACCAAGGTCGAGATTAAG |
| 139112-aa | 81 | QVQLVESGGGLVQPGGSLRLSCAVSGFALSNHGMSWVRRAPGKGLE |
| VH | | WVSGIVYSGSTYYAASVKGRFTISRDNSRNTLYLQMNSLRPEDTAIYY |
| | | CSAHGGESDVWGQGTTVTVSS |
| 139112-aa | 96 | DIRLTQSPSPLSASVGDRVTITCQASEDINKFLNWYHQTPGKAPKLLIY |
| VL | | DASTLQTGVPSRFSGSGSGTDFTLTINSLQPEDIGTYYCQQYESLPLTFG |
| | | GGTKVEIK |
| 139112-aa | 111 | MALPVTALLLPLALLLHAARPQVQLVESGGGLVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSDIRLTQSPSPLSASVGDRVTITCQASEDINKFLNWYHQ |
| | | TPGKAPKLLIYDASTLQTGVPSRFSGSGSGTDFTLTINSLQPEDIGTYYC |
| | | QQYESLPLTFGGGTKVEIKTTTPAPRPPTPAPTIASQPLSLRPEACRPAA |
| | | GGAVHTRGLDFACDIYIWAPLAGTCGVLLLSLVITLYCKRGRKKLLYIF |
| | | KQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQ |
| | | KDKMAEAYSEIGMKGERRRGKGHDGLYQGLSTATKDTYDALHMQAL |
| | | PPR |
| 139112-nt | 126 | ATGGCCCTCCCTGTCACCGCCCTGCTGCTTCCGCTGGCTCTTCTGCT |
| Full CAR | | CCACGCCGCTCGGCCCCAAGTGCAACTCGTGGAATCTGGTGGAGGA |
| | | CTCGTGCAACCCGGTGGAAGCCTTAGGCTGTCGTGCGCCGTCAGCG |
| | | GGTTTGCTCTGAGCAACCATGGAATGTCCTGGGTCCGCCGGGCACC |
| | | GGGAAAAGGGCTGGAATGGGTGTCCGGCATCGTGTACAGCGGGTC |
| | | AACCTATTACGCCGCGTCCGTGAAGGGCAGATTCACTATCTCAAGA |
| | | GACAACAGCCGGAACACCCTGTACTTGCAAATGAATTCCCTGCGCC |
| | | CCGAGGACACCGCCATCTACTACTGCTCCGCCCACGGAGGAGAGTC |
| | | GGACGTGTGGGGCCAGGGAACGACTGTGACTGTGTCCAGCGCATCA |
| | | GGAGGGGGTGGTTCGGGCGGCCGGGCCTCGGGGGGAGGAGGTTCC |
| | | GACATTCGGCTGACCCAGTCCCCGTCCCCACTGTCGGCCTCCGTCGG |
| | | CGACCGCGTGACCATCACTTGTCAGGCGTCCGAGGACATTAACAAG |
| | | TTCCTGAACTGGTACCACCAGACCCCTGGAAAGGCCCCCAAGCTGC |
| | | TGATCTACGATGCCTCGACCCTTCAAACTGGAGTGCCTAGCCGGTTC |
| | | TCCGGGTCCGGCTCCGGCACTGATTTCACTCTGACCATCAACTCATT |
| | | GCAGCCGGAAGATATCGGGACCTACTATTGCCAGCAGTACGAATCC |
| | | CTCCCGCTCACATTCGGCGGGGGAACCAAGGTCGAGATTAAGACCA |
| | | CTACCCCAGCACCGAGGCCACCCACCCCGGCTCCTACCATCGCCTC |
| | | CCAGCCTCTGTCCCTGCGTCCGGAGGCATGTAGACCCGCAGCTGGT |
| | | GGGGCCGTGCATACCCGGGGTCTTGACTTCGCCTGCGATATCTACA |
| | | TTTGGGCCCCTCTGGCTGGTACTTGCGGGGTCCTGCTGCTTTCACTC |
| | | GTGATCACTCTTTACTGTAAGCGCGGTCGGAAGAAGCTGCTGTACA |
| | | TCTTTAAGCAACCCTTCATGAGGCCTGTGCAGACTACTCAAGAGGA |
| | | GGACGGCTGTTCATGCCGGTTCCCAGAGGAGGAGGAAGGCGGCTG |
| | | CGAACTGCGCGTGAAATTCAGCCGCAGCGCAGATGCTCCAGCCTAC |
| | | AAGCAGGGGCAGAACCAGCTCTACAACGAACTCAATCTTGGTCGGA |
| | | GAGAGGAGTACGACGTGCTGGACAAGCGGAGAGGACGGGACCCAG |
| | | AAATGGGCGGGAAGCCGCGCAGAAAGAATCCCCAAGAGGGCCTGT |
| | | ACAACGAGCTCCAAAAGGATAAGATGGCAGAAGCCTATAGCGAGA |
| | | TTGGTATGAAAGGGGAACGCAGAAGAGGCAAAGGCCACGACGGAC |
| | | TGTACCAGGGACTCAGCACCGCCACCAAGGACACCTATGACGCTCT |
| | | TCACATGCAGGCCCTGCCGCCTCGG |

139113

| | | |
|---|---|---|
| 139113-aa | 52 | EVQLVETGGGLVQPGGSLRLSCAVSGFALSNHGMSWVRRAPGKGLE |
| ScFv domain | | WVSGIVYSGSTYYAASVKGRFTISRDNSRNTLYLQMNSLRPEDTAIYY |
| | | CSAHGGESDVWGQGTTVTVSSASGGGGSGGRASGGGGSETTLTQSPA |
| | | TLSVSPGERATLSCRASQSVGSNLAWYQQKPGQGPRLLIYGASTRATGI |
| | | PARFSGSGSGTEFTLTISSLQPEDFAVYYCQQYNDWLPVTFGQGTKVEI |
| | | K |
| 139113-nt | 67 | GAAGTGCAATTGGTGGAAACTGGAGGAGGACTTGTGCAACCTGGA |
| ScFv domain | | GGATCATTGCGGCTCTCATGCGCTGTCTCCGGCTTCGCCCTGTCAAA |
| | | TCACGGGATGTCGTGGGTCAGACGGGCCCCGGGAAAGGGTCTGGA |
| | | ATGGGTGTCGGGGATTGTGTACAGCGGCTCCACCTACTACGCCGCT |
| | | TCGGTCAAGGGCCGCTTCACTATTTCACGGGACAACAGCCGCAACA |
| | | CCCTCTATCTGCAAATGAACTCTCTCCGCCCGGAGGATACCGCCATC |
| | | TACTACTGCTCCGCACACGGCGGCGAATCCGACGTGTGGGGACAGG |
| | | GAACCACTGTCACCGTGTCGTCCGCATCCGGTGGCGGAGGATCGGG |
| | | TGGCCGGGCCTCCGGGGGCGGCGGCAGCGAGACTACCCTGACCCA |
| | | GTCCCCTGCCACTCTGTCCGTGAGCCCGGGAGAGAGAGCCACCCTT |
| | | AGCTGCCGGGCCAGCCAGAGCGTGGGCTCCAACCTGGCCTGGTACC |
| | | AGCAGAAGCCAGGACAGGGTCCCAGGCTGCTGATCTACGGAGCCTC |
| | | CACTCGCGCGACCGGCATCCCCGCGAGGTTCTCCGGGTCGGGTTCC |
| | | GGGACCGAGTTCACCCTGACCATCTCCTCCCTCCAACCGGAGGACT |
| | | TCGCGGTGTACTACTGTCAGCAGTACAACGATTGGCTGCCCGTGAC |
| | | ATTTGGACAGGGGACGAAGGTGGAAATCAAA |
| 139113-aa | 82 | EVQLVETGGGLVQPGGSLRLSCAVSGFALSNHGMSWVRRAPGKGLE |
| VH | | WVSGIVYSGSTYYAASVKGRFTISRDNSRNTLYLQMNSLRPEDTAIYY |
| | | CSAHGGESDVWGQGTTVTVSS |
| 139113-aa | 97 | ETTLTQSPATLSVSPGERATLSCRASQSVGSNLAWYQQKPGQGPRLLIY |
| VL | | GASTRATGIPARFSGSGSGTEFTLTISSLQPEDFAVYYCQQYNDWLPVT |
| | | FGQGTKVEIK |
| 139113-aa | 112 | MALPVTALLLPLALLLHAARPEVQLVETGGGLVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSETTLTQSPATLSVSPGERATLSCRASQSVGSNLAWYQ |
| | | QKPGQGPRLLIYGASTRATGIPARFSGSGSGTEFTLTISSLQPEDFAVYY |
| | | CQQYNDWLPVTFGQGTKVEIKTTTPAPRPPTPAPTIASQPLSLRPEACRP |
| | | AAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 139113-nt | 127 | ATGGCCCTCCCTGTCACCGCCCTGCTGCTTCCGCTGGCTCTTCTGCT |
| Full CAR | | CCACGCCGCTCGGCCCGAAGTGCAATTGGTGGAAACTGGAGGAGG |
| | | ACTTGTGCAACCTGGAGGATCATTGCGGCTCTCATGCGCTGTCTCCG |
| | | GCTTCGCCCTGTCAAATCACGGGATGTCGTGGGTCAGACGGGCCCC |
| | | GGGAAAGGGTCTGGAATGGGTGTCGGGGATTGTGTACAGCGGCTCC |
| | | ACCTACTACGCCGCTTCGGTCAAGGGCCGCTTCACTATTTCACGGG |
| | | ACAACAGCCGCAACACCCTCTATCTGCAAATGAACTCTCTCCGCCC |
| | | GGAGGATACCGCCATCTACTACTGCTCCGCACACGGCGGCGAATCC |
| | | GACGTGTGGGGACAGGGAACCACTGTCACCGTGTCGTCCGCATCCG |
| | | GTGGCGGAGGATCGGGTGGCCGGGCCTCCGGGGGCGGCGGCAGCG |
| | | AGACTACCCTGACCCAGTCCCCTGCCACTCTGTCCGTGAGCCCGGG |
| | | AGAGAGAGCCACCCTTAGCTGCCGGGCCAGCCAGAGCGTGGGCTCC |
| | | AACCTGGCCTGGTACCAGCAGAAGCCAGGACAGGGTCCCAGGCTG |
| | | CTGATCTACGGAGCCTCCACTCGCGCGACCGGCATCCCCGCGAGGT |
| | | TCTCCGGGTCGGGTTCCGGGACCGAGTTCACCCTGACCATCTCCTCC |
| | | CTCCAACCGGAGGACTTCGCGGTGTACTACTGTCAGCAGTACAACG |
| | | ATTGGCTGCCCGTGACATTTGGACAGGGGACGAAGGTGGAAATCAA |
| | | AACCACTACCCCAGCACCGAGGCCACCCACCCCGGCTCCTACCATC |
| | | GCCTCCCAGCCTCTGTCCCTGCGTCCGGAGGCATGTAGACCCGCAG |
| | | CTGGTGGGGCCGTGCATACCCGGGGTCTTGACTTCGCCTGCGATAT |
| | | CTACATTTGGGCCCCTCTGGCTGGTACTTGCGGGGTCCTGCTGCTTT |
| | | CACTCGTGATCACTCTTTACTGTAAGCGCGGTCGGAAGAAGCTGCT |
| | | GTACATCTTTAAGCAACCCTTCATGAGGCCTGTGCAGACTACTCAA |
| | | GAGGAGGACGGCTGTTCATGCCGGTTCCCAGAGGAGGAGGAAGGC |
| | | GGCTGCGAACTGCGCGTGAAATTCAGCCGCAGCGCAGATGCTCCAG |
| | | CCTACAAGCAGGGGCAGAACCAGCTCTACAACGAACTCAATCTTGG |
| | | TCGGAGAGAGGAGTACGACGTGCTGGACAAGCGGAGAGGACGGGA |
| | | CCCAGAAATGGGCGGGAAGCCGCGCAGAAAGAATCCCCAAGAGGG |
| | | CCTGTACAACGAGCTCCAAAAGGATAAGATGGCAGAAGCCTATAG |
| | | CGAGATTGGTATGAAAGGGGAACGCAGAAGAGGCAAAGGCCACGA |
| | | CGGACTGTACCAGGGACTCAGCACCGCCACCAAGGACACCTATGAC |
| | | GCTCTTCACATGCAGGCCCTGCCGCCTCGG |

139114

| | | |
|---|---|---|
| 139114-aa | 53 | EVQLVESGGGLVQPGGSLRLSCAVSGFALSNHGMSWVRRAPGKGLE |
| ScFv domain | | WVSGIVYSGSTYYAASVKGRFTISRDNSRNTLYLQMNSLRPEDTAIYY |
| | | CSAHGGESDVWGQGTTVTVSSASGGGGSGGRASGGGGSEIVLTQSPG |
| | | TLSLSPGERATLSCRASQSIGSSSLAWYQQKPGQAPRLLMYGASSRASG |
| | | IPDRFSGSGSGTDFTLTISRLEPEDFAVYYCQQYAGSPPFTFGQGTKVEI |
| | | K |
| 139114-nt | 68 | GAAGTGCAATTGGTGGAATCTGGTGGAGGACTTGTGCAACCTGGAG |
| ScFv domain | | GATCACTGAGACTGTCATGCGCGGTGTCCGGTTTTGCCCTGAGCAA |
| | | TCATGGGATGTCGTGGGTCCGGCGCGCCCCCGGAAAGGGTCTGGAA |
| | | TGGGTGTCGGGTATCGTCTACTCCGGGAGCACTTACTACGCCGCGA |
| | | GCGTGAAGGGCCGCTTCACCATTTCCCGCGATAACTCCCGCAACAC |
| | | CCTGTACTTGCAAATGAACTCGCTCCGGCCTGAGGACACTGCCATC |
| | | TACTACTGCTCCGCACACGGAGGAGAATCCGACGTGTGGGGCCAGG |
| | | GAACTACCGTGACCGTCAGCAGCGCCTCCGGCGGCGGGGGCTCAGG |
| | | CGGACGGGCTAGCGGCGGCGGTGGCTCCGAGATCGTGCTGACCCAG |
| | | TCGCCTGGCACTCTCTCGCTGAGCCCCGGGGAAAGGGCAACCCTGT |
| | | CCTGTCGGGCCAGCCAGTCCATTGGATCATCCTCCCTCGCCTGGTAT |
| | | CAGCAGAAACCGGGACAGGCTCCGCGGCTGCTTATGTATGGGGCCA |
| | | GCTCAAGAGCCTCCGGCATTCCCGACCGGTTCTCCGGGTCCGGTTCC |
| | | GGCACCGATTTCACCCTGACTATCTCGAGGCTGGAGCCAGAGGACT |
| | | TCGCCGTGTACTACTGCCAGCAGTACGCGGGGTCCCCGCCGTTCAC |
| | | GTTCGGACAGGGAACCAAGGTCGAGATCAAG |
| 139114-aa | 83 | EVQLVESGGGLVQPGGSLRLSCAVSGFALSNHGMSWVRRAPGKGLE |
| VH | | WVSGIVYSGSTYYAASVKGRFTISRDNSRNTLYLQMNSLRPEDTAIYY |
| | | CSAHGGESDVWGQGTTVTVSS |
| 139114-aa | 98 | EIVLTQSPGTLSLSPGERATLSCRASQSIGSSSLAWYQQKPGQAPRLLM |
| VL | | YGASSRASGIPDRFSGSGSGTDFTLTISRLEPEDFAVYYCQQYAGSPPFT |
| | | FGQGTKVEIK |
| 139114-aa | 113 | MALPVTALLLPLALLLHAARPEVQLVESGGGLVQPGGSLRLSCAVSGF |
| Full CAR | | ALSNHGMSWVRRAPGKGLEWVSGIVYSGSTYYAASVKGRFTISRDNS |
| | | RNTLYLQMNSLRPEDTAIYYCSAHGGESDVWGQGTTVTVSSASGGGG |
| | | SGGRASGGGGSEIVLTQSPGTLSLSPGERATLSCRASQSIGSSSLAWYQ |
| | | QKPGQAPRLLMYGASSRASGIPDRFSGSGSGTDFTLTISRLEPEDFAVY |
| | | YCQQYAGSPPFTFGQGTKVEIKTTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 139114-nt | 128 | ATGGCCCTCCCTGTCACCGCCCTGCTGCTTCCGCTGGCTCTTCTGCT |
| Full CAR | | CCACGCCGCTCGGCCCGAAGTGCAATTGGTGGAATCTGGTGGAGGA |
| | | CTTGTGCAACCTGGAGGATCACTGAGACTGTCATGCGCGGTGTCCG |
| | | GTTTTGCCCTGAGCAATCATGGGATGTCGTGGGTCCGGCGCGCCCC |
| | | CGGAAAGGGTCTGGAATGGGTGTCGGGTATCGTCTACTCCGGGAGC |
| | | ACTTACTACGCCGCGAGCGTGAAGGGCCGCTTCACCATTTCCCGCG |
| | | ATAACTCCCGCAACACCCTGTACTTGCAAATGAACTCGCTCCGGCC |
| | | TGAGGACACTGCCATCTACTACTGCTCCGCACACGGAGGAGAATCC |
| | | GACGTGTGGGGCCAGGGAACTACCGTGACCGTCAGCAGCGCCTCCG |
| | | GCGGCGGGGGCTCAGGCGGACGGGCTAGCGGCGGCGGTGGCTCCG |
| | | AGATCGTGCTGACCCAGTCGCCTGGCACTCTCTCGCTGAGCCCCGG |
| | | GGAAAGGGCAACCCTGTCCTGTCGGGCCAGCCAGTCCATTGGATCA |
| | | TCCTCCCTCGCCTGGTATCAGCAGAAACCGGGACAGGCTCCGCGGC |
| | | TGCTTATGTATGGGGCCAGCTCAAGAGCCTCCGGCATTCCCGACCG |
| | | GTTCTCCGGGTCCGGTTCCGGCACCGATTTCACCCTGACTATCTCGA |
| | | GGCTGGAGCCAGAGGACTTCGCCGTGTACTACTGCCAGCAGTACGC |
| | | GGGGTCCCCGCCGTTCACGTTCGGACAGGGAACCAAGGTCGAGATC |
| | | AAGACCACTACCCCAGCACCGAGGCCACCCACCCCGGCTCCTACCA |
| | | TCGCCTCCCAGCCTCTGTCCCTGCGTCCGGAGGCATGTAGACCCGC |
| | | AGCTGGTGGGGCCGTGCATACCCGGGGTCTTGACTTCGCCTGCGAT |
| | | ATCTACATTTGGGCCCCTCTGGCTGGTACTTGCGGGGTCCTGCTGCT |
| | | TTCACTCGTGATCACTCTTTACTGTAAGCGCGGTCGGAAGAAGCTG |
| | | CTGTACATCTTTAAGCAACCCTTCATGAGGCCTGTGCAGACTACTCA |
| | | AGAGGAGGACGGCTGTTCATGCCGGTTCCCAGAGGAGGAGGAAGG |
| | | CGGCTGCGAACTGCGCGTGAAATTCAGCCGCAGCGCAGATGCTCCA |
| | | GCCTACAAGCAGGGGCAGAACCAGCTCTACAACGAACTCAATCTTG |
| | | GTCGGAGAGAGGAGTACGACGTGCTGGACAAGCGGAGAGGACGGG |
| | | ACCCAGAAATGGGCGGGAAGCCGCGCAGAAAGAATCCCCAAGAGG |
| | | GCCTGTACAACGAGCTCCAAAAGGATAAGATGGCAGAAGCCTATA |
| | | GCGAGATTGGTATGAAAGGGGAACGCAGAAGAGGCAAAGGCCACG |
| | | ACGGACTGTACCAGGGACTCAGCACCGCCACCAAGGACACCTATGA |
| | | CGCTCTTCACATGCAGGCCCTGCCGCCTCGG |

149362

| | | |
|---|---|---|
| 149362-aa | 129 | QVQLQESGPGLVKPSETLSLTCTVSGGSISSSYYYWGWIRQPPGKGLE |
| ScFv domain | | WIGSIYYSGSAYYNPSLKSRVTISVDTSKNQFSLRLSSVTAADTAVYYC |
| | | ARHWQEWPDAFDIWGQGTMVTVSSGGGGSGGGGSGGGGSETTLTQS |
| | | PAFMSATPGDKVIISCKASQDIDDAMNWYQQKPGEAPLFIIQSATSPVP |
| | | GIPPRFSGSGFGTDFSLTINNIESEDAAYYFCLQHDNFPLTFGQGTKLEIK |
| 149362-nt | 150 | CAAGTGCAGCTTCAGGAAAGCGGACCGGGCCTGGTCAAGCCATCCG |
| ScFv domain | | AAACTCTCTCCCTGACTTGCACTGTGTCTGGCGGTTCCATCTCATCG |
| | | TCGTACTACTACTGGGGCTGGATTAGGCAGCCGCCCGGAAAGGGAC |
| | | TGGAGTGGATCGGAAGCATCTACTATTCCGGCTCGGCGTACTACAA |
| | | CCCTAGCCTCAAGTCGAGAGTGACCATCTCCGTGGATACCTCCAAG |
| | | AACCAGTTTTCCCTGCGCCTGAGCTCCGTGACCGCCGCTGACACCG |
| | | CCGTGTACTACTGTGCTCGGCATTGGCAGGAATGGCCCGATGCCTT |
| | | CGACATTTGGGGCCAGGGCACTATGGTCACTGTGTCATCCGGGGGT |
| | | GGAGGCAGCGGGGGAGGAGGGTCCGGGGGGGGAGGTTCAGAGAC |
| | | AACCTTGACCCAGTCACCCGCATTCATGTCCGCCACTCCGGGAGAC |
| | | AAGGTCATCATCTCGTGCAAAGCGTCCCAGGATATCGACGATGCCA |
| | | TGAATTGGTACCAGCAGAAGCCTGGCGAAGCGCCGCTGTTCATTAT |
| | | CCAATCCGCAACCTCGCCCGTGCCTGGAATCCCACCGCGGTTCAGC |
| | | GGCAGCGGTTTCGGAACCGACTTTTCCCTGACCATTAACAACATTG |
| | | AGTCCGAGGACGCCGCCTACTACTTCTGCCTGCAACACGACAACTT |
| | | CCCTCTCACGTTCGGCCAGGGAACCAAGCTGGAAATCAAG |
| 149362-aa | 171 | QVQLQESGPGLVKPSETLSLTCTVSGGSISSSYYYWGWIRQPPGKGLE |
| VH | | WIGSIYYSGSAYYNPSLKSRVTISVDTSKNQFSLRLSSVTAADTAVYYC |
| | | ARHWQEWPDAFDIWGQGTMVTVSS |
| 149362-aa | 192 | ETTLTQSPAFMSATPGDKVIISCKASQDIDDAMNWYQQKPGEAPLFIIQ |
| VL | | SATSPVPGIPPRFSGSGFGTDFSLTINNIESEDAAYYFCLQHDNFPLTFGQ |
| | | GTKLEIK |
| 149362-aa | 213 | MALPVTALLLPLALLLHAARPQVQLQESGPGLVKPSETLSLTCTVSGGS |
| Full CAR | | ISSSYYYWGWIRQPPGKGLEWIGSIYYSGSAYYNPSLKSRVTISVDTSK |
| | | NQFSLRLSSVTAADTAVYYCARHWQEWPDAFDIWGQGTMVTVSSGG |
| | | GGSGGGGSGGGGSETTLTQSPAFMSATPGDKVIISCKASQDIDDAMNW |
| | | YQQKPGEAPLFIIQSATSPVPGIPPRFSGSGFGTDFSLTINNIESEDAAYY |
| | | FCLQHDNFPLTFGQGTKLEIKTTTPAPRPPTPAPTIASQPLSLRPEACRPA |
| | | AGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQ |
| | | ALPPR |
| 149362-nt | 234 | ATGGCCCTCCCTGTCACCGCCCTGCTGCTTCCGCTGGCTCTTCTGCT |
| Full CAR | | CCACGCCGCTCGGCCCCAAGTGCAGCTTCAGGAAAGCGGACCGGGC |
| | | CTGGTCAAGCCATCCGAAACTCTCTCCCTGACTTGCACTGTGTCTGG |
| | | CGGTTCCATCTCATCGTCGTACTACTACTGGGGCTGGATTAGGCAGC |
| | | CGCCCGGAAAGGGACTGGAGTGGATCGGAAGCATCTACTATTCCGG |
| | | CTCGGCGTACTACAACCCTAGCCTCAAGTCGAGAGTGACCATCTCC |
| | | GTGGATACCTCCAAGAACCAGTTTTCCCTGCGCCTGAGCTCCGTGA |
| | | CCGCCGCTGACACCGCCGTGTACTACTGTGCTCGGCATTGGCAGGA |
| | | ATGGCCCGATGCCTTCGACATTTGGGGCCAGGGCACTATGGTCACT |
| | | GTGTCATCCGGGGGTGGAGGCAGCGGGGGAGGAGGGTCCGGGGGG |
| | | GGAGGTTCAGAGACAACCTTGACCCAGTCACCCGCATTCATGTCCG |
| | | CCACTCCGGGAGACAAGGTCATCATCTCGTGCAAAGCGTCCCAGGA |
| | | TATCGACGATGCCATGAATTGGTACCAGCAGAAGCCTGGCGAAGCG |
| | | CCGCTGTTCATTATCCAATCCGCAACCTCGCCCGTGCCTGGAATCCC |
| | | ACCGCGGTTCAGCGGCAGCGGTTTCGGAACCGACTTTTCCCTGACC |
| | | ATTAACAACATTGAGTCCGAGGACGCCGCCTACTACTTCTGCCTGC |
| | | AACACGACAACTTCCCTCTCACGTTCGGCCAGGGAACCAAGCTGGA |
| | | AATCAAGACCACTACCCCAGCACCGAGGCCACCCACCCCGGCTCCT |
| | | ACCATCGCCTCCCAGCCTCTGTCCCTGCGTCCGGAGGCATGTAGAC |
| | | CCGCAGCTGGTGGGGCCGTGCATACCCGGGGTCTTGACTTCGCCTG |
| | | CGATATCTACATTTGGGCCCCTCTGGCTGGTACTTGCGGGGTCCTGC |
| | | TGCTTTCACTCGTGATCACTCTTTACTGTAAGCGCGGTCGGAAGAAG |
| | | CTGCTGTACATCTTTAAGCAACCCTTCATGAGGCCTGTGCAGACTAC |
| | | TCAAGAGGAGGACGGCTGTTCATGCCGGTTCCCAGAGGAGGAGGA |
| | | AGGCGGCTGCGAACTGCGCGTGAAATTCAGCCGCAGCGCAGATGCT |
| | | CCAGCCTACAAGCAGGGGCAGAACCAGCTCTACAACGAACTCAATC |
| | | TTGGTCGGAGAGAGGAGTACGACGTGCTGGACAAGCGGAGAGGAC |
| | | GGGACCCAGAAATGGGCGGGAAGCCGCGCAGAAAGAATCCCCAAG |
| | | AGGGCCTGTACAACGAGCTCCAAAAGGATAAGATGGCAGAAGCCT |
| | | ATAGCGAGATTGGTATGAAAGGGGAACGCAGAAGAGGCAAAGGCC |
| | | ACGACGGACTGTACCAGGGACTCAGCACCGCCACCAAGGACACCT |
| | | ATGACGCTCTTCACATGCAGGCCCTGCCGCCTCGG |

149363

| | | |
|---|---|---|
| 149363-aa | 130 | VNLRESGPALVKPTQTLTLTCTFSGFSLRTSGMCVSWIRQPPGKALEW |
| ScFv domain | | LARIDWDEDKFYSTSLKTRLTISKDTSDNQVVLRMTNMDPADTATYY |
| | | CARSGAGGTSATAFDIWGPGTMVTVSSGGGGSGGGGSGGGGSDIQMT |
| | | QSPSSLSASVGDRVTITCRASQDIYNNLAWFQLKPGSAPRSLMYAANK |
| | | SQSGVPSRFSGSASGTDFTLTISSLQPEDFATYYCQHYYRFPYSFGQGT |
| | | KLEIK |
| 149363-nt | 151 | CAAGTCAATCTGCGCGAATCCGGCCCCGCCTTGGTCAAGCCTACCC |
| ScFv domain | | AGACCCTCACTCTGACCTGTACTTTCTCCGGCTTCTCCCTGCGGACT |
| | | TCCGGGATGTGCGTGTCCTGGATCAGACAGCCTCCGGGAAAGGCCC |
| | | TGGAGTGGCTCGCTCGCATTGACTGGGATGAGGACAAGTTCTACTC |
| | | CACCTCACTCAAGACCAGGCTGACCATCAGCAAAGATACCTCTGAC |
| | | AACCAAGTGGTGCTCCGCATGACCAACATGGACCCAGCCGACACTG |
| | | CCACTTACTACTGCGCGAGGAGCGGAGCGGGCGGAACCTCCGCCAC |
| | | CGCCTTCGATATTTGGGGCCCGGGTACCATGGTCACCGTGTCAAGC |
| | | GGAGGAGGGGGGTCCGGGGGCGGCGGTTCCGGGGGAGGCGGATCG |
| | | GACATTCAGATGACTCAGTCACCATCGTCCCTGAGCGCTAGCGTGG |
| | | GCGACAGAGTGACAATCACTTGCCGGGCATCCCAGGACATCTATAA |
| | | CAACCTTGCGTGGTTCCAGCTGAAGCCTGGTTCCGCACCGCGGTCA |
| | | CTTATGTACGCCGCCAACAAGAGCCAGTCGGGAGTGCCGTCCCGGT |
| | | TTTCCGGTTCGGCCTCGGGAACTGACTTCACCCTGACGATCTCCAGC |
| | | CTGCAACCCGAGGATTTCGCCACCTACTACTGCCAGCACTACTACC |
| | | GCTTTCCCTACTCGTTCGGACAGGGAACCAAGCTGGAAATCAAG |
| 149363-aa | 172 | QVNLRESGPALVKPTQTLTLTCTFSGFSLRTSGMCVSWIRQPPGKALE |
| VH | | WLARIDWDEDKFYSTSLKTRLTISKDTSDNQVVLRMTNMDPADTATY |
| | | YCARSGAGGTSATAFDIWGPGTMVTVSS |
| 149363-aa | 193 | DIQMTQSPSSLSASVGDRVTITCRASQDIYNNLAWFQLKPGSAPRSLMY |
| VL | | AANKSQSGVPSRFSGSASGTDFTLTISSLQPEDFATYYCQHYYRFPYSF |
| | | GQGTKLEIK |
| 149363-aa | 214 | MALPVTALLLPLALLLHAARPQVNLRESGPALVKPTQTLTLTCTFSGFS |
| Full CAR | | LRTSGMCVSWIRQPPGKALEWLARIDWDEDKFYSTSLKTRLTISKDTS |
| | | DNQVVLRMTNMDPADTATYYCARSGAGGTSATAFDIWGPGTMVTVS |
| | | SGGGGSGGGGSGGGGSDIQMTQSPSSLSASVGDRVTITCRASQDIYNN |
| | | LAWFQLKPGSAPRSLMYAANKSQSGVPSRFSGSASGTDFTLTISSLQPE |
| | | DFATYYCQHYYRFPYSFGQGTKLEIKTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYD |
| | | ALHMQALPPR |
| 149363-nt | 235 | ATGGCCCTCCCTGTCACCGCCCTGCTGCTTCCGCTGGCTCTTCTGCT |
| Full CAR | | CCACGCCGCTCGGCCCCAAGTCAATCTGCGCGAATCCGGCCCCGCC |
| | | TTGGTCAAGCCTACCCAGACCCTCACTCTGACCTGTACTTTCTCCGG |
| | | CTTCTCCCTGCGGACTTCCGGGATGTGCGTGTCCTGGATCAGACAGC |
| | | CTCCGGGAAAGGCCCTGGAGTGGCTCGCTCGCATTGACTGGGATGA |
| | | GGACAAGTTCTACTCCACCTCACTCAAGACCAGGCTGACCATCAGC |
| | | AAAGATACCTCTGACAACCAAGTGGTGCTCCGCATGACCAACATGG |
| | | ACCCAGCCGACACTGCCACTTACTACTGCGCGAGGAGCGGAGCGGG |
| | | CGGAACCTCCGCCACCGCCTTCGATATTTGGGGCCCGGGTACCATG |
| | | GTCACCGTGTCAAGCGGAGGAGGGGGGTCCGGGGGCGGCGGTTCC |
| | | GGGGGAGGCGGATCGGACATTCAGATGACTCAGTCACCATCGTCCC |
| | | TGAGCGCTAGCGTGGGCGACAGAGTGACAATCACTTGCCGGGCATC |
| | | CCAGGACATCTATAACAACCTTGCGTGGTTCCAGCTGAAGCCTGGT |
| | | TCCGCACCGCGGTCACTTATGTACGCCGCCAACAAGAGCCAGTCGG |
| | | GAGTGCCGTCCCGGTTTTCCGGTTCGGCCTCGGGAACTGACTTCACC |
| | | CTGACGATCTCCAGCCTGCAACCCGAGGATTTCGCCACCTACTACT |
| | | GCCAGCACTACTACCGCTTTCCCTACTCGTTCGGACAGGGAACCAA |
| | | GCTGGAAATCAAGACCACTACCCCAGCACCGAGGCCACCCACCCCG |
| | | GCTCCTACCATCGCCTCCCAGCCTCTGTCCCTGCGTCCGGAGGCATG |
| | | TAGACCCGCAGCTGGTGGGGCCGTGCATACCCGGGGTCTTGACTTC |
| | | GCCTGCGATATCTACATTTGGGCCCCTCTGGCTGGTACTTGCGGGGT |
| | | CCTGCTGCTTTCACTCGTGATCACTCTTTACTGTAAGCGCGGTCGGA |
| | | AGAAGCTGCTGTACATCTTTAAGCAACCCTTCATGAGGCCTGTGCA |
| | | GACTACTCAAGAGGAGGACGGCTGTTCATGCCGGTTCCCAGAGGAG |
| | | GAGGAAGGCGGCTGCGAACTGCGCGTGAAATTCAGCCGCAGCGCA |
| | | GATGCTCCAGCCTACAAGCAGGGGCAGAACCAGCTCTACAACGAA |
| | | CTCAATCTTGGTCGGAGAGAGGAGTACGACGTGCTGGACAAGCGG |
| | | AGAGGACGGGACCCAGAAATGGGCGGGAAGCCGCGCAGAAAGAAT |
| | | CCCCAAGAGGGCCTGTACAACGAGCTCCAAAAGGATAAGATGGCA |
| | | GAAGCCTATAGCGAGATTGGTATGAAAGGGGAACGCAGAAGAGGC |
| | | AAAGGCCACGACGGACTGTACCAGGGACTCAGCACCGCCACCAAG |
| | | GACACCTATGACGCTCTTCACATGCAGGCCCTGCCGCCTCGG |

149364

| | | |
|---|---|---|
| 149364-aa | 131 | EVQLVESGGGLVKPGGSLRLSCAASGFTFSSYSMNWVRQAPGKGLEW |
| ScFv domain | | VSSISSSSSYIYYADSVKGRFTISRDNAKNSLYLQMNSLRAEDTAVYYC |
| | | AKTIAAVYAFDIWGQGTTVTVSSGGGGSGGGGSGGGGSEIVLTQSPLS |
| | | LPVTPEEPASISCRSSQSLLHSNGYNYLDWYLQKPGQSPQLLIYLGSNR |
| | | ASGVPDRFSGSGSGTDFTLKISRVEAEDVGVYYCMQALQTPYTFGQGT |
| | | KLEIK |
| 149364-nt | 152 | GAAGTGCAGCTTGTCGAATCCGGGGGGGGACTGGTCAAGCCGGGC |
| ScFv domain | | GGATCACTGAGACTGTCCTGCGCCGCGAGCGGCTTCACGTTCTCCTC |
| | | CTACTCCATGAACTGGGTCCGCCAAGCCCCCGGGAAGGGACTGGAA |
| | | TGGGTGTCCTCTATCTCCTCGTCGTCGTCCTACATCTACTACGCCGA |
| | | CTCCGTGAAGGGAAGATTCACCATTTCCCGCGACAACGCAAAGAAC |
| | | TCACTGTACTTGCAAATGAACTCACTCCGGGCCGAAGATACTGCTG |
| | | TGTACTATTGCGCCAAGACTATTGCCGCCGTCTACGCTTTCGACATC |
| | | TGGGGCCAGGGAACCACCGTGACTGTGTCGTCCGGTGGTGGTGGCT |
| | | CGGGCGGAGGAGGAAGCGGCGGCGGGGGGTCCGAGATTGTGCTGA |
| | | CCCAGTCGCCACTGAGCCTCCCTGTGACCCCCGAGGAACCCGCCAG |
| | | CATCAGCTGCCGGTCCAGCCAGTCCCTGCTCCACTCCAACGGATAC |
| | | AATTACCTCGATTGGTACCTTCAGAAGCCTGGACAAAGCCCGCAGC |
| | | TGCTCATCTACTTGGGATCAAACCGCGCGTCAGGAGTGCCTGACCG |
| | | GTTCTCCGGCTCGGGCAGCGGTACCGATTTCACCCTGAAAATCTCC |
| | | AGGGTGGAGGCAGAGGACGTGGGAGTGTATTACTGTATGCAGGCG |
| | | CTGCAGACTCCGTACACATTTGGGCAGGGCACCAAGCTGGAGATCA |
| | | AG |
| 149364-aa | 173 | EVQLVESGGGLVKPGGSLRLSCAASGFTFSSYSMNWVRQAPGKGLEW |
| VH | | VSSISSSSSYIYYADSVKGRFTISRDNAKNSLYLQMNSLRAEDTAVYYC |
| | | AKTIAAVYAFDIWGQGTTVTVSS |
| 149364-aa | 194 | EIVLTQSPLSLPVTPEEPASISCRSSQSLLHSNGYNYLDWYLQKPGQSPQ |
| VL | | LLIYLGSNRASGVPDRFSGSGSGTDFTLKISRVEAEDVGVYYCMQALQ |
| | | TPYTFGQGTKLEIK |
| 149364-aa | 215 | MALPVTALLLPLALLLHAARPEVQLVESGGGLVKPGGSLRLSCAASGF |
| Full CAR | | TFSSYSMNWVRQAPGKGLEWVSSISSSSSYIYYADSVKGRFTISRDNAK |
| | | NSLYLQMNSLRAEDTAVYYCAKTIAAVYAFDIWGQGTTVTVSSGGGG |
| | | SGGGGSGGGGSEIVLTQSPLSLPVTPEEPASISCRSSQSLLHSNGYNYLD |
| | | WYLQKPGQSPQLLIYLGSNRASGVPDRFSGSGSGTDFTLKISRVEAEDV |
| | | GVYYCMQALQTPYTFGQGTKLEIKTTTPAPRPPTPAPTIASQPLSLRPEA |
| | | CRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGL |
| | | YNELQKDKMAEAYSEIGMKGERRRGKGHDGLYQGLSTATKDTYDAL |
| | | HMQALPPR |
| 149364-nt | 236 | ATGGCCCTCCCTGTCACCGCCCTGCTGCTTCCGCTGGCTCTTCTGCT |
| Full CAR | | CCACGCCGCTCGGCCCGAAGTGCAGCTTGTCGAATCCGGGGGGGGA |
| | | CTGGTCAAGCCGGGCGGATCACTGAGACTGTCCTGCGCCGCGAGCG |
| | | GCTTCACGTTCTCCTCCTACTCCATGAACTGGGTCCGCCAAGCCCCC |
| | | GGGAAGGGACTGGAATGGGTGTCCTCTATCTCCTCGTCGTCGTCCT |
| | | ACATCTACTACGCCGACTCCGTGAAGGGAAGATTCACCATTTCCCG |
| | | CGACAACGCAAAGAACTCACTGTACTTGCAAATGAACTCACTCCGG |
| | | GCCGAAGATACTGCTGTGTACTATTGCGCCAAGACTATTGCCGCCG |
| | | TCTACGCTTTCGACATCTGGGGCCAGGGAACCACCGTGACTGTGTC |
| | | GTCCGGTGGTGGTGGCTCGGGCGGAGGAGGAAGCGGCGGCGGGGG |
| | | GTCCGAGATTGTGCTGACCCAGTCGCCACTGAGCCTCCCTGTGACC |
| | | CCCGAGGAACCCGCCAGCATCAGCTGCCGGTCCAGCCAGTCCCTGC |
| | | TCCACTCCAACGGATACAATTACCTCGATTGGTACCTTCAGAAGCCT |
| | | GGACAAAGCCCGCAGCTGCTCATCTACTTGGGATCAAACCGCGCGT |
| | | CAGGAGTGCCTGACCGGTTCTCCGGCTCGGGCAGCGGTACCGATTT |
| | | CACCCTGAAAATCTCCAGGGTGGAGGCAGAGGACGTGGGAGTGTA |
| | | TTACTGTATGCAGGCGCTGCAGACTCCGTACACATTTGGGCAGGGC |
| | | ACCAAGCTGGAGATCAAGACCACTACCCCAGCACCGAGGCCACCC |
| | | ACCCCGGCTCCTACCATCGCCTCCCAGCCTCTGTCCCTGCGTCCGGA |
| | | GGCATGTAGACCCGCAGCTGGTGGGGCCGTGCATACCCGGGGTCTT |
| | | GACTTCGCCTGCGATATCTACATTTGGGCCCCTCTGGCTGGTACTTG |
| | | CGGGGTCCTGCTGCTTTCACTCGTGATCACTCTTTACTGTAAGCGCG |
| | | GTCGGAAGAAGCTGCTGTACATCTTTAAGCAACCCTTCATGAGGCC |
| | | TGTGCAGACTACTCAAGAGGAGGACGGCTGTTCATGCCGGTTCCCA |
| | | GAGGAGGAGGAAGGCGGCTGCGAACTGCGCGTGAAATTCAGCCGC |
| | | AGCGCAGATGCTCCAGCCTACAAGCAGGGGCAGAACCAGCTCTAC |
| | | AACGAACTCAATCTTGGTCGGAGAGAGGAGTACGACGTGCTGGAC |
| | | AAGCGGAGAGGACGGGACCCAGAAATGGGCGGGAAGCCGCGCAG |
| | | AAAGAATCCCCAAGAGGGCCTGTACAACGAGCTCCAAAAGGATAA |
| | | GATGGCAGAAGCCTATAGCGAGATTGGTATGAAAGGGGAACGCAG |
| | | AAGAGGCAAAGGCCACGACGGACTGTACCAGGGACTCAGCACCGC |
| | | CACCAAGGACACCTATGACGCTCTTCACATGCAGGCCCTGCCGCCT |
| | | CGG |

149365

| | | |
|---|---|---|
| 149365-aa | 132 | EVQLVESGGGLVKPGGSLRLSCAASGFTFSDYYMSWIRQAPGKGLEW |
| ScFv domain | | VSYISSSGSTIYYADSVKGRFTISRDNAKNSLYLQMNSLRAEDTAVYYC |
| | | ARDLRGAFDIWGQGTMVTVSSGGGGSGGGGSGGGGSSYVLTQSPSVS |
| | | AAPGYTATISCGGNNIGTKSVHWYQQKPGQAPLLVIRDDSVRPSKIPGR |
| | | FSGSNSGNMATLTISGVQAGDEADFYCQVWDSDSEHVVFGGGTKLTV |
| | | L |
| 149365-nt | 153 | GAAGTCCAGCTCGTGGAGTCCGGCGGAGGCCTTGTGAAGCCTGGAG |
| ScFv domain | | GTTCGCTGAGACTGTCCTGCGCCGCCTCCGGCTTCACCTTCTCCGAC |
| | | TACTACATGTCCTGGATCAGACAGGCCCCGGGAAAGGGCCTGGAAT |
| | | GGGTGTCCTACATCTCGTCATCGGGCAGCACTATCTACTACGCGGA |
| | | CTCAGTGAAGGGGCGGTTCACCATTTCCCGGGATAACGCGAAGAAC |
| | | TCGCTGTATCTGCAAATGAACTCACTGAGGGCCGAGGACACCGCCG |
| | | TGTACTACTGCGCCCGCGATCTCCGCGGGGCATTTGACATCTGGGG |
| | | ACAGGGAACCATGGTCACAGTGTCCAGCGGAGGGGGAGGATCGGG |
| | | TGGCGGAGGTTCCGGGGGTGGAGGCTCCTCCTACGTGCTGACTCAG |
| | | AGCCCAAGCGTCAGCGCTGCGCCCGGTTACACGGCAACCATCTCCT |
| | | GTGGCGGAAACAACATTGGGACCAAGTCTGTGCACTGGTATCAGCA |
| | | GAAGCCGGGCCAAGCTCCCCTGTTGGTGATCCGCGATGACTCCGTG |
| | | CGGCCTAGCAAAATTCCGGGACGGTTCTCCGGCTCCAACAGCGGCA |
| | | ATATGGCCACTCTCACCATCTCGGGAGTGCAGGCCGGAGATGAAGC |
| | | CGACTTCTACTGCCAAGTCTGGGACTCAGACTCCGAGCATGTGGTG |
| | | TTCGGGGGCGGAACCAAGCTGACTGTGCTC |
| 149365-aa | 174 | EVQLVESGGGLVKPGGSLRLSCAASGFTFSDYYMSWIRQAPGKGLEW |
| VH | | VSYISSSGSTIYYADSVKGRFTISRDNAKNSLYLQMNSLRAEDTAVYYC |
| | | ARDLRGAFDIWGQGTMVTVSS |
| 149365-aa | 195 | SYVLTQSPSVSAAPGYTATISCGGNNIGTKSVHWYQQKPGQAPLLVIR |
| VL | | DDSVRPSKIPGRFSGSNSGNMATLTISGVQAGDEADFYCQVWDSDSEH |
| | | VVFGGGTKLTVL |
| 149365-aa | 216 | MALPVTALLLPLALLLHAARPEVQLVESGGGLVKPGGSLRLSCAASGF |
| Full CAR | | TFSDYYMSWIRQAPGKGLEWVSYISSSGSTIYYADSVKGRFTISRDNAK |
| | | NSLYLQMNSLRAEDTAVYYCARDLRGAFDIWGQGTMVTVSSGGGGS |
| | | GGGGSGGGGSSYVLTQSPSVSAAPGYTATISCGGNNIGTKSVHWYQQ |
| | | KPGQAPLLVIRDDSVRPSKIPGRFSGSNSGNMATLTISGVQAGDEADFY |
| | | CQVWDSDSEHVVFGGGTKLTVLTTTPAPRPPTPAPTIASQPLSLRPEAC |
| | | RPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLY |
| | | NELQKDKMAEAYSEIGMKGERRRGKGHDGLYQGLSTATKDTYDALH |
| | | MQALPPR |
| 149365-nt | 237 | ATGGCCCTCCCTGTCACCGCCCTGCTGCTTCCGCTGGCTCTTCTGCT |
| Full CAR | | CCACGCCGCTCGGCCCGAAGTCCAGCTCGTGGAGTCCGGCGGAGGC |
| | | CTTGTGAAGCCTGGAGGTTCGCTGAGACTGTCCTGCGCCGCCTCCG |
| | | GCTTCACCTTCTCCGACTACTACATGTCCTGGATCAGACAGGCCCCG |
| | | GGAAAGGGCCTGGAATGGGTGTCCTACATCTCGTCATCGGGCAGCA |
| | | CTATCTACTACGCGGACTCAGTGAAGGGGCGGTTCACCATTTCCCG |
| | | GGATAACGCGAAGAACTCGCTGTATCTGCAAATGAACTCACTGAGG |
| | | GCCGAGGACACCGCCGTGTACTACTGCGCCCGCGATCTCCGCGGGG |
| | | CATTTGACATCTGGGGACAGGGAACCATGGTCACAGTGTCCAGCGG |
| | | AGGGGGAGGATCGGGTGGCGGAGGTTCCGGGGGTGGAGGCTCCTC |
| | | CTACGTGCTGACTCAGAGCCCAAGCGTCAGCGCTGCGCCCGGTTAC |
| | | ACGGCAACCATCTCCTGTGGCGGAAACAACATTGGGACCAAGTCTG |
| | | TGCACTGGTATCAGCAGAAGCCGGGCCAAGCTCCCCTGTTGGTGAT |
| | | CCGCGATGACTCCGTGCGGCCTAGCAAAATTCCGGGACGGTTCTCC |
| | | GGCTCCAACAGCGGCAATATGGCCACTCTCACCATCTCGGGAGTGC |
| | | AGGCCGGAGATGAAGCCGACTTCTACTGCCAAGTCTGGGACTCAGA |
| | | CTCCGAGCATGTGGTGTTCGGGGGCGGAACCAAGCTGACTGTGCTC |
| | | ACCACTACCCCAGCACCGAGGCCACCCACCCCGGCTCCTACCATCG |
| | | CCTCCCAGCCTCTGTCCCTGCGTCCGGAGGCATGTAGACCCGCAGC |
| | | TGGTGGGGCCGTGCATACCCGGGGTCTTGACTTCGCCTGCGATATCT |
| | | ACATTTGGGCCCCTCTGGCTGGTACTTGCGGGGTCCTGCTGCTTTCA |
| | | CTCGTGATCACTCTTTACTGTAAGCGCGGTCGGAAGAAGCTGCTGT |
| | | ACATCTTTAAGCAACCCTTCATGAGGCCTGTGCAGACTACTCAAGA |
| | | GGAGGACGGCTGTTCATGCCGGTTCCCAGAGGAGGAGGAAGGCGG |
| | | CTGCGAACTGCGCGTGAAATTCAGCCGCAGCGCAGATGCTCCAGCC |
| | | TACAAGCAGGGGCAGAACCAGCTCTACAACGAACTCAATCTTGGTC |
| | | GGAGAGAGGAGTACGACGTGCTGGACAAGCGGAGAGGACGGGACC |
| | | CAGAAATGGGCGGGAAGCCGCGCAGAAAGAATCCCCAAGAGGGCC |
| | | TGTACAACGAGCTCCAAAAGGATAAGATGGCAGAAGCCTATAGCG |
| | | AGATTGGTATGAAAGGGGAACGCAGAAGAGGCAAAGGCCACGACG |
| | | GACTGTACCAGGGACTCAGCACCGCCACCAAGGACACCTATGACGC |
| | | TCTTCACATGCAGGCCCTGCCGCCTCGG |

149366

| | | |
|---|---|---|
| 149366-aa | 133 | QVQLVQSGAEVKKPGASVKVSCKPSGYTVTSHYIHWVRRAPGQGLE |
| ScFv domain | | WMGMINPSGGVTAYSQTLQGRVTMTSDTSSSTVYMELSSLRSEDTAM |
| | | YYCAREGSGSGWYFDFWGRGTLVTVSSGGGGSGGGGSGGGGSSYVL |
| | | TQPPSVSVSPGQTASITCSGDGLSKKYVSWYQQKAGQSPVVLISRDKE |
| | | RPSGIPDRFSGSNSADTATLTISGTQAMDEADYYCQAWDDTTVVFGGG |
| | | TKLTVL |
| 149366-nt | 154 | CAAGTGCAGCTGGTGCAGAGCGGGGCCGAAGTCAAGAAGCCGGGA |
| ScFv domain | | GCCTCCGTGAAAGTGTCCTGCAAGCCTTCGGGATACACCGTGACCT |
| | | CCCACTACATTCATTGGGTCCGCCGCGCCCCCGGCCAAGGACTCGA |
| | | GTGGATGGGCATGATCAACCCTAGCGGCGGAGTGACCGCGTACAGC |
| | | CAGACGCTGCAGGGACGCGTGACTATGACCTCGGATACCTCCTCCT |
| | | CCACCGTCTATATGGAACTGTCCAGCCTGCGGTCCGAGGATACCGC |
| | | CATGTACTACTGCGCCCGGGAAGGATCAGGCTCCGGGTGGTATTTC |
| | | GACTTCTGGGGAAGAGGCACCCTCGTGACTGTGTCATCTGGGGGAG |
| | | GGGGTTCCGGTGGTGGCGGATCGGGAGGAGGCGGTTCATCCTACGT |
| | | GCTGACCCAGCCACCCTCCGTGTCCGTGAGCCCCGGCCAGACTGCA |
| | | TCGATTACATGTAGCGGCGACGGCCTCTCCAAGAAATACGTGTCGT |
| | | GGTACCAGCAGAAGGCCGGACAGAGCCCGGTGGTGCTGATCTCAA |
| | | GAGATAAGGAGCGGCCTAGCGGAATCCCGGACAGGTTCTCGGGTTC |
| | | CAACTCCGCGGACACTGCTACTCTGACCATCTCGGGGACCCAGGCT |
| | | ATGGACGAAGCCGATTACTACTGCCAAGCCTGGGACGACACTACTG |
| | | TCGTGTTTGGAGGGGGCACCAAGTTGACCGTCCTT |
| 149366-aa | 175 | QVQLVQSGAEVKKPGASVKVSCKPSGYTVTSHYIHWVRRAPGQGLE |
| VH | | WMGMINPSGGVTAYSQTLQGRVTMTSDTSSSTVYMELSSLRSEDTAM |
| | | YYCAREGSGSGWYFDFWGRGTLVTVSS |
| 149366-aa | 196 | SYVLTQPPSVSVSPGQTASITCSGDGLSKKYVSWYQQKAGQSPVVLISR |
| VL | | DKERPSGIPDRFSGSNSADTATLTISGTQAMDEADYYCQAWDDTTVVF |
| | | GGGTKLTVL |
| 149366-aa | 217 | MALPVTALLLPLALLLHAARPQVQLVQSGAEVKKPGASVKVSCKPSG |
| Full CAR | | YTVTSHYIHWVRRAPGQGLEWMGMINPSGGVTAYSQTLQGRVTMTS |
| | | DTSSSTVYMELSSLRSEDTAMYYCAREGSGSGWYFDFWGRGTLVTVS |
| | | SGGGGSGGGGSGGGGSSYVLTQPPSVSVSPGQTASITCSGDGLSKKYV |
| | | SWYQQKAGQSPVVLISRDKERPSGIPDRFSGSNSADTATLTISGTQAMD |
| | | EADYYCQAWDDTTVVFGGGTKLTVLTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYD |
| | | ALHMQALPPR |
| 149366-nt | 238 | ATGGCCCTCCCTGTCACCGCCCTGCTGCTTCCGCTGGCTCTTCTGCT |
| Full CAR | | CCACGCCGCTCGGCCCCAAGTGCAGCTGGTGCAGAGCGGGGCCGA |
| | | AGTCAAGAAGCCGGGAGCCTCCGTGAAAGTGTCCTGCAAGCCTTCG |
| | | GGATACACCGTGACCTCCCACTACATTCATTGGGTCCGCCGCGCCC |
| | | CCGGCCAAGGACTCGAGTGGATGGGCATGATCAACCCTAGCGGCG |
| | | GAGTGACCGCGTACAGCCAGACGCTGCAGGGACGCGTGACTATGA |
| | | CCTCGGATACCTCCTCCTCCACCGTCTATATGGAACTGTCCAGCCTG |
| | | CGGTCCGAGGATACCGCCATGTACTACTGCGCCCGGGAAGGATCAG |
| | | GCTCCGGGTGGTATTTCGACTTCTGGGGAAGAGGCACCCTCGTGAC |
| | | TGTGTCATCTGGGGGAGGGGGTTCCGGTGGTGGCGGATCGGGAGGA |
| | | GGCGGTTCATCCTACGTGCTGACCCAGCCACCCTCCGTGTCCGTGA |
| | | GCCCCGGCCAGACTGCATCGATTACATGTAGCGGCGACGGCCTCTC |
| | | CAAGAAATACGTGTCGTGGTACCAGCAGAAGGCCGGACAGAGCCC |
| | | GGTGGTGCTGATCTCAAGAGATAAGGAGCGGCCTAGCGGAATCCCG |
| | | GACAGGTTCTCGGGTTCCAACTCCGCGGACACTGCTACTCTGACCA |
| | | TCTCGGGGACCCAGGCTATGGACGAAGCCGATTACTACTGCCAAGC |
| | | CTGGGACGACACTACTGTCGTGTTTGGAGGGGGCACCAAGTTGACC |
| | | GTCCTTACCACTACCCCAGCACCGAGGCCACCCACCCCGGCTCCTA |
| | | CCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCG |
| | | ATATCTACATTTGGGCCCCTCTGGCTGGTACTTGCGGGGTCCTGCTG |
| | | CTTTCACTCGTGATCACTCTTTACTGTAAGCGCGGTCGGAAGAAGCT |
| | | GCTGTACATCTTTAAGCAACCCTTCATGAGGCCTGTGCAGACTACTC |
| | | AAGAGGAGGACGGCTGTTCATGCCGGTTCCCAGAGGAGGAGGAAG |
| | | GCGGCTGCGAACTGCGCGTGAAATTCAGCCGCAGCGCAGATGCTCC |
| | | AGCCTACAAGCAGGGGCAGAACCAGCTCTACAACGAACTCAATCTT |
| | | GGTCGGAGAGAGGAGTACGACGTGCTGGACAAGCGGAGAGGACGG |
| | | GACCCAGAAATGGGCGGGAAGCCGCGCAGAAAGAATCCCCAAGAG |
| | | GGCCTGTACAACGAGCTCCAAAAGGATAAGATGGCAGAAGCCTAT |
| | | AGCGAGATTGGTATGAAAGGGGAACGCAGAAGAGGCAAAGGCCAC |
| | | GACGGACTGTACCAGGGACTCAGCACCGCCACCAAGGACACCTATG |
| | | ACGCTCTTCACATGCAGGCCCTGCCGCCTCGG |

149367

| | | |
|---|---|---|
| 149367-aa | 134 | QVQLQESGPGLVKPSQTLSLTCTVSGGSISSGGYYWSWIRQHPGKGLE |
| ScFv domain | | WIGYIYYSGSTYYNPSLKSRVTISVDTSKNQFSLKLSSVTAADTAVYYC |
| | | ARAGIAARLRGAFDIWGQGTMVTVSSGGGGSGGGGSGGGGSDIVMTQ |
| | | SPSSVSASVGDRVIITCRASQGIRNWLAWYQQKPGKAPNLLIYAASNL |
| | | QSGVPSRFSGSGSGADFTLTISSLQPEDVATYYCQKYNSAPFTFGPGTK |
| | | VDIK |
| 149367-nt | 155 | CAAGTGCAGCTTCAGGAGAGCGGCCCGGGACTCGTGAAGCCGTCCC |
| ScFv domain | | AGACCCTGTCCCTGACTTGCACCGTGTCGGGAGGAAGCATCTCGAG |
| | | CGGAGGCTACTATTGGTCGTGGATTCGGCAGCACCCTGGAAAGGGC |
| | | CTGGAATGGATCGGCTACATCTACTACTCCGGCTCGACCTACTACA |
| | | ACCCATCGCTGAAGTCCAGAGTGACAATCTCAGTGGACACGTCCAA |
| | | GAATCAGTTCAGCCTGAAGCTCTCTTCCGTGACTGCGGCCGACACC |
| | | GCCGTGTACTACTGCGCACGCGCTGGAATTGCCGCCCGGCTGAGGG |
| | | GTGCCTTCGACATTTGGGGACAGGGCACCATGGTCACCGTGTCCTC |
| | | CGGCGGCGGAGGTTCCGGGGGTGGAGGCTCAGGAGGAGGGGGGTC |
| | | CGACATCGTCATGACTCAGTCGCCCTCAAGCGTCAGCGCGTCCGTC |
| | | GGGGACAGAGTGATCATCACCTGTCGGGCGTCCCAGGGAATTCGCA |
| | | ACTGGCTGGCCTGGTATCAGCAGAAGCCCGGAAAGGCCCCCAACCT |
| | | GTTGATCTACGCCGCCTCAAACCTCCAATCCGGGGTGCCGAGCCGC |
| | | TTCAGCGGCTCCGGTTCGGGTGCCGATTTCACTCTGACCATCTCCTC |
| | | CCTGCAACCTGAAGATGTGGCTACCTACTACTGCCAAAAGTACAAC |
| | | TCCGCACCTTTTACTTTCGGACCGGGGACCAAAGTGGACATTAAG |
| 149367-aa | 176 | QVQLQESGPGLVKPSQTLSLTCTVSGGSISSGGYYWSWIRQHPGKGLE |
| VH | | WIGYIYYSGSTYYNPSLKSRVTISVDTSKNQFSLKLSSVTAADTAVYYC |
| | | ARAGIAARLRGAFDIWGQGTMVTVSS |
| 149367-aa | 197 | DIVMTQSPSSVSASVGDRVIITCRASQGIRNWLAWYQQKPGKAPNLLIY |
| VL | | AASNLQSGVPSRFSGSGSGADFTLTISSLQPEDVATYYCQKYNSAPFTF |
| | | GPGTKVDIK |
| 149367-aa | 218 | MALPVTALLLPLALLLHAARPQVQLQESGPGLVKPSQTLSLTCTVSGG |
| Full CAR | | SISSGGYYWSWIRQHPGKGLEWIGYIYYSGSTYYNPSLKSRVTISVDTS |
| | | KNQFSLKLSSVTAADTAVYYCARAGIAARLRGAFDIWGQGTMVTVSS |
| | | GGGGSGGGGSGGGGSDIVMTQSPSSVSASVGDRVIITCRASQGIRNWL |
| | | AWYQQKPGKAPNLLIYAASNLQSGVPSRFSGSGSGADFTLTISSLQPED |
| | | VATYYCQKYNSAPFTFGPGTKVDIKTTTPAPRPPTPAPTIASQPLSLRPE |
| | | ACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAP |
| | | AYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDA |
| | | LHMQALPPR |
| 149367-nt | 239 | ATGGCCCTCCCTGTCACCGCCCTGCTGCTTCCGCTGGCTCTTCTGCT |
| Full CAR | | CCACGCCGCTCGGCCCCAAGTGCAGCTTCAGGAGAGCGGCCCGGGA |
| | | CTCGTGAAGCCGTCCCAGACCCTGTCCCTGACTTGCACCGTGTCGG |
| | | GAGGAAGCATCTCGAGCGGAGGCTACTATTGGTCGTGGATTCGGCA |
| | | GCACCCTGGAAAGGGCCTGGAATGGATCGGCTACATCTACTACTCC |
| | | GGCTCGACCTACTACAACCCATCGCTGAAGTCCAGAGTGACAATCT |
| | | CAGTGGACACGTCCAAGAATCAGTTCAGCCTGAAGCTCTCTTCCGT |
| | | GACTGCGGCCGACACCGCCGTGTACTACTGCGCACGCGCTGGAATT |
| | | GCCGCCCGGCTGAGGGGTGCCTTCGACATTTGGGGACAGGGCACCA |
| | | TGGTCACCGTGTCCTCCGGCGGCGGAGGTTCCGGGGGTGGAGGCTC |
| | | AGGAGGAGGGGGGTCCGACATCGTCATGACTCAGTCGCCCTCAAGC |
| | | GTCAGCGCGTCCGTCGGGGACAGAGTGATCATCACCTGTCGGGCGT |
| | | CCCAGGGAATTCGCAACTGGCTGGCCTGGTATCAGCAGAAGCCCGG |
| | | AAAGGCCCCCAACCTGTTGATCTACGCCGCCTCAAACCTCCAATCC |
| | | GGGGTGCCGAGCCGCTTCAGCGGCTCCGGTTCGGGTGCCGATTTCA |
| | | CTCTGACCATCTCCTCCCTGCAACCTGAAGATGTGGCTACCTACTAC |
| | | TGCCAAAAGTACAACTCCGCACCTTTTACTTTCGGACCGGGGACCA |
| | | AAGTGGACATTAAGACCACTACCCCAGCACCGAGGCCACCCACCCC |
| | | GGCTCCTACCATCGCCTCCCAGCCTCTGTCCCTGCGTCCGGAGGCAT |
| | | GTAGACCCGCAGCTGGTGGGGCCGTGCATACCCGGGGTCTTGACTT |
| | | CGCCTGCGATATCTACATTTGGGCCCCTCTGGCTGGTACTTGCGGGG |
| | | TCCTGCTGCTTTCACTCGTGATCACTCTTTACTGTAAGCGCGGTCGG |
| | | AAGAAGCTGCTGTACATCTTTAAGCAACCCTTCATGAGGCCTGTGC |
| | | AGACTACTCAAGAGGAGGACGGCTGTTCATGCCGGTTCCCAGAGGA |
| | | GGAGGAAGGCGGCTGCGAACTGCGCGTGAAATTCAGCCGCAGCGC |
| | | AGATGCTCCAGCCTACAAGCAGGGGCAGAACCAGCTCTACAACGA |
| | | ACTCAATCTTGGTCGGAGAGAGGAGTACGACGTGCTGGACAAGCG |
| | | GAGAGGACGGGACCCAGAAATGGGCGGGAAGCCGCGCAGAAAGA |
| | | ATCCCCAAGAGGGCCTGTACAACGAGCTCCAAAAGGATAAGATGG |
| | | CAGAAGCCTATAGCGAGATTGGTATGAAAGGGGAACGCAGAAGAG |
| | | GCAAAGGCCACGACGGACTGTACCAGGGACTCAGCACCGCCACCA |
| | | AGGACACCTATGACGCTCTTCACATGCAGGCCCTGCCGCCTCGG |

149368

| | | |
|---|---|---|
| 149368-aa | 135 | QVQLVQSGAEVKKPGSSVKVSCKASGGTFSSYAISWVRQAPGQGLEW |
| ScFv domain | | MGGIIPIFGTANYAQKFQGRVTITADESTSTAYMELSSLRSEDTAVYYC |
| | | ARRGGYQLLRWDVGLLRSAFDIWGQGTMVTVSSGGGGSGGGGSGGG |
| | | GSSYVLTQPPSVSVAPGQTARITCGGNNIGSKSVHWYQQKPGQAPVLV |
| | | LYGKNNRPSGVPDRFSGSRSGTTASLTITGAQAEDEADYYCSSRDSSG |
| | | DHLRVFGTGTKVTVL |
| 149368-nt | 156 | CAAGTGCAGCTGGTCCAGTCGGGCGCCGAGGTCAAGAAGCCCGGG |
| ScFv domain | | AGCTCTGTGAAAGTGTCCTGCAAGGCCTCCGGGGGCACCTTTAGCT |
| | | CCTACGCCATCTCCTGGGTCCGCCAAGCACCGGGTCAAGGCCTGGA |
| | | GTGGATGGGGGGAATTATCCCTATCTTCGGCACTGCCAACTACGCC |
| | | CAGAAGTTCCAGGGACGCGTGACCATTACCGCGGACGAATCCACCT |
| | | CCACCGCTTATATGGAGCTGTCCAGCTTGCGCTCGGAAGATACCGC |
| | | CGTGTACTACTGCGCCCGGAGGGGTGGATACCAGCTGCTGAGATGG |
| | | GACGTGGGCCTCCTGCGGTCGGCGTTCGACATCTGGGGCCAGGGCA |
| | | CTATGGTCACTGTGTCCAGCGGAGGAGGCGGATCGGGAGGCGGCG |
| | | GATCAGGGGGAGGCGGTTCCAGCTACGTGCTTACTCAACCCCCTTC |
| | | GGTGTCCGTGGCCCCGGGACAGACCGCCAGAATCACTTGCGGAGGA |
| | | AACAACATTGGGTCCAAGAGCGTGCATTGGTACCAGCAGAAGCCA |
| | | GGACAGGCCCCTGTGCTGGTGCTCTACGGGAAGAACAATCGGCCCA |
| | | GCGGAGTGCCGGACAGGTTCTCGGGTTCACGCTCCGGTACAACCGC |
| | | TTCACTGACTATCACCGGGGCCCAGGCAGAGGATGAAGCGGACTAC |
| | | TACTGTTCCTCCCGGGATTCATCCGGCGACCACCTCCGGGTGTTCGG |
| | | AACCGGAACGAAGGTCACCGTGCTG |
| 149368-aa | 177 | QVQLVQSGAEVKKPGSSVKVSCKASGGTFSSYAISWVRQAPGQGLEW |
| VH | | MGGIIPIFGTANYAQKFQGRVTITADESTSTAYMELSSLRSEDTAVYYC |
| | | ARRGGYQLLRWDVGLLRSAFDIWGQGTMVTVSS |
| 149368-aa | 198 | SYVLTQPPSVSVAPGQTARITCGGNNIGSKSVHWYQQKPGQAPVLVLY |
| VL | | GKNNRPSGVPDRFSGSRSGTTASLTITGAQAEDEADYYCSSRDSSGDH |
| | | LRVFGTGTKVTVL |
| 149368-aa | 219 | MALPVTALLLPLALLLHAARPQVQLVQSGAEVKKPGSSVKVSCKASG |
| Full CAR | | GTFSSYAISWVRQAPGQGLEWMGGIIPIFGTANYAQKFQGRVTITADES |
| | | TSTAYMELSSLRSEDTAVYYCARRGGYQLLRWDVGLLRSAFDIWGQG |
| | | TMVTVSSGGGGSGGGGSGGGGSSYVLTQPPSVSVAPGQTARITCGGN |
| | | NIGSKSVHWYQQKPGQAPVLVLYGKNNRPSGVPDRFSGSRSGTTASLT |
| | | ITGAQAEDEADYYCSSRDSSGDHLRVFGTGTKVTVLTTTPAPRPPTPAP |
| | | TIASQPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGGCEL |
| | | RVKFSRSADAPAYKQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR |
| 149368-nt | 240 | ATGGCCCTCCCTGTCACCGCCCTGCTGCTTCCGCTGGCTCTTCTGCT |
| Full CAR | | CCACGCCGCTCGGCCCCAAGTGCAGCTGGTCCAGTCGGGCGCCGAG |
| | | GTCAAGAAGCCCGGGAGCTCTGTGAAAGTGTCCTGCAAGGCCTCCG |
| | | GGGGCACCTTTAGCTCCTACGCCATCTCCTGGGTCCGCCAAGCACC |
| | | GGGTCAAGGCCTGGAGTGGATGGGGGGAATTATCCCTATCTTCGGC |
| | | ACTGCCAACTACGCCCAGAAGTTCCAGGGACGCGTGACCATTACCG |
| | | CGGACGAATCCACCTCCACCGCTTATATGGAGCTGTCCAGCTTGCG |
| | | CTCGGAAGATACCGCCGTGTACTACTGCGCCCGGAGGGGTGGATAC |
| | | CAGCTGCTGAGATGGGACGTGGGCCTCCTGCGGTCGGCGTTCGACA |
| | | TCTGGGGCCAGGGCACTATGGTCACTGTGTCCAGCGGAGGAGGCGG |
| | | ATCGGGAGGCGGCGGATCAGGGGGAGGCGGTTCCAGCTACGTGCTT |
| | | ACTCAACCCCCTTCGGTGTCCGTGGCCCCGGGACAGACCGCCAGAA |
| | | TCACTTGCGGAGGAAACAACATTGGGTCCAAGAGCGTGCATTGGTA |
| | | CCAGCAGAAGCCAGGACAGGCCCCTGTGCTGGTGCTCTACGGGAAG |
| | | AACAATCGGCCCAGCGGAGTGCCGGACAGGTTCTCGGGTTCACGCT |
| | | CCGGTACAACCGCTTCACTGACTATCACCGGGGCCCAGGCAGAGGA |
| | | TGAAGCGGACTACTACTGTTCCTCCCGGGATTCATCCGGCGACCAC |
| | | CTCCGGGTGTTCGGAACCGGAACGAAGGTCACCGTGCTGACCACTA |
| | | CCCCAGCACCGAGGCCACCCACCCCGGCTCCTACCATCGCCTCCCA |
| | | GCCTCTGTCCCTGCGTCCGGAGGCATGTAGACCCGCAGCTGGTGGG |
| | | GCCGTGCATACCCGGGGTCTTGACTTCGCCTGCGATATCTACATTTG |
| | | GGCCCCTCTGGCTGGTACTTGCGGGGTCCTGCTGCTTTCACTCGTGA |
| | | TCACTCTTTACTGTAAGCGCGGTCGGAAGAAGCTGCTGTACATCTTT |
| | | AAGCAACCCTTCATGAGGCCTGTGCAGACTACTCAAGAGGAGGACG |
| | | GCTGTTCATGCCGGTTCCCAGAGGAGGAGGAAGGCGGCTGCGAACT |
| | | GCGCGTGAAATTCAGCCGCAGCGCAGATGCTCCAGCCTACAAGCAG |
| | | GGGCAGAACCAGCTCTACAACGAACTCAATCTTGGTCGGAGAGAG |
| | | GAGTACGACGTGCTGGACAAGCGGAGAGGACGGGACCCAGAAATG |
| | | GGCGGGAAGCCGCGCAGAAAGAATCCCCAAGAGGGCCTGTACAAC |
| | | GAGCTCCAAAAGGATAAGATGGCAGAAGCCTATAGCGAGATTGGT |
| | | ATGAAAGGGGAACGCAGAAGAGGCAAAGGCCACGACGGACTGTAC |
| | | CAGGGACTCAGCACCGCCACCAAGGACACCTATGACGCTCTTCACA |
| | | TGCAGGCCCTGCCGCCTCGG |

149369

| | | |
|---|---|---|
| 149369-aa | 136 | EVQLQQSGPGLVKPSQTLSLTCAISGDSVSSNSAAWNWIRQSPSRGLE |
| ScFv domain | | WLGRTYYRSKWYSFYAISLKSRIIINPDTSKNQFSLQLKSVTPEDTAVY |
| | | YCARSSPEGLFLYWFDPWGQGTLVTVSSGGDGSGGGGSGGGGSSSEL |
| | | TQDPAVSVALGQTIRITCQGDSLGNYYATWYQQKPGQAPVLVIYGTN |
| | | NRPSGIPDRFSASSSGNTASLTITGAQAEDEADYYCNSRDSSGHHLLFG |
| | | TGTKVTVL |
| 149369-nt | 157 | GAAGTGCAGCTCCAACAGTCAGGACCGGGGCTCGTGAAGCCATCCC |
| ScFv domain | | AGACCCTGTCCCTGACTTGTGCCATCTCGGGAGATAGCGTGTCATC |
| | | GAACTCCGCCGCCTGGAACTGGATTCGGCAGAGCCCGTCCCGCGGA |
| | | CTGGAGTGGCTTGGAAGGACCTACTACCGGTCCAAGTGGTACTCTT |
| | | TCTACGCGATCTCGCTGAAGTCCCGCATTATCATTAACCCTGATACC |
| | | TCCAAGAATCAGTTCTCCCTCCAACTGAAATCCGTCACCCCCGAGG |
| | | ACACAGCAGTGTATTACTGCGCACGGAGCAGCCCCGAAGGACTGTT |
| | | CCTGTATTGGTTTGACCCCTGGGGCCAGGGGACTCTTGTGACCGTGT |
| | | CGAGCGGCGGAGATGGGTCCGGTGGCGGTGGTTCGGGGGGCGGCG |
| | | GATCATCATCCGAACTGACCCAGGACCCGGCTGTGTCCGTGGCGCT |
| | | GGGACAAACCATCCGCATTACGTGCCAGGGAGACTCCCTGGGCAAC |
| | | TACTACGCCACTTGGTACCAGCAGAAGCCGGGCCAAGCCCCTGTGT |
| | | TGGTCATCTACGGGACCAACAACAGACCTTCCGGCATCCCCGACCG |
| | | GTTCAGCGCTTCGTCCTCCGGCAACACTGCCAGCCTGACCATCACTG |
| | | GAGCGCAGGCCGAAGATGAGGCCGACTACTACTGCAACAGCAGAG |
| | | ACTCCTCGGGTCATCACCTCTTGTTCGGAACTGGAACCAAGGTCAC |
| | | CGTGCTG |
| 149369-aa | 178 | EVQLQQSGPGLVKPSQTLSLTCAISGDSVSSNSAAWNWIRQSPSRGLE |
| VH | | WLGRTYYRSKWYSFYAISLKSRIIINPDTSKNQFSLQLKSVTPEDTAVY |
| | | YCARSSPEGLFLYWFDPWGQGTLVTVSS |
| 149369-aa | 199 | SSELTQDPAVSVALGQTIRITCQGDSLGNYYATWYQQKPGQAPVLVIY |
| VL | | GTNNRPSGIPDRFSASSSGNTASLTITGAQAEDEADYYCNSRDSSGHHL |
| | | LFGTGTKVTVL |
| 149369-aa | 220 | MALPVTALLLPLALLLHAARPEVQLQQSGPGLVKPSQTLSLTCAISGDS |
| Full CAR | | VSSNSAAWNWIRQSPSRGLEWLGRTYYRSKWYSFYAISLKSRIIINPDT |
| | | SKNQFSLQLKSVTPEDTAVYYCARSSPEGLFLYWFDPWGQGTLVTVSS |
| | | GGDGSGGGGSGGGGSSSELTQDPAVSVALGQTIRITCQGDSLGNYYAT |
| | | WYQQKPGQAPVLVIYGTNNRPSGIPDRFSASSSGNTASLTITGAQAEDE |
| | | ADYYCNSRDSSGHHLLFGTGTKVTVLTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYD |
| | | ALHMQALPPR |
| 149369-nt | 241 | ATGGCCCTCCCTGTCACCGCCCTGCTGCTTCCGCTGGCTCTTCTGCT |
| Full CAR | | CCACGCCGCTCGGCCCGAAGTGCAGCTCCAACAGTCAGGACCGGGG |
| | | CTCGTGAAGCCATCCCAGACCCTGTCCCTGACTTGTGCCATCTCGGG |
| | | AGATAGCGTGTCATCGAACTCCGCCGCCTGGAACTGGATTCGGCAG |
| | | AGCCCGTCCCGCGGACTGGAGTGGCTTGGAAGGACCTACTACCGGT |
| | | CCAAGTGGTACTCTTTCTACGCGATCTCGCTGAAGTCCCGCATTATC |
| | | ATTAACCCTGATACCTCCAAGAATCAGTTCTCCCTCCAACTGAAATC |
| | | CGTCACCCCCGAGGACACAGCAGTGTATTACTGCGCACGGAGCAGC |
| | | CCCGAAGGACTGTTCCTGTATTGGTTTGACCCCTGGGGCCAGGGGA |
| | | CTCTTGTGACCGTGTCGAGCGGCGGAGATGGGTCCGGTGGCGGTGG |
| | | TTCGGGGGGCGGCGGATCATCATCCGAACTGACCCAGGACCCGGCT |
| | | GTGTCCGTGGCGCTGGGACAAACCATCCGCATTACGTGCCAGGGAG |
| | | ACTCCCTGGGCAACTACTACGCCACTTGGTACCAGCAGAAGCCGGG |
| | | CCAAGCCCCTGTGTTGGTCATCTACGGGACCAACAACAGACCTTCC |
| | | GGCATCCCCGACCGGTTCAGCGCTTCGTCCTCCGGCAACACTGCCA |
| | | GCCTGACCATCACTGGAGCGCAGGCCGAAGATGAGGCCGACTACTA |
| | | CTGCAACAGCAGAGACTCCTCGGGTCATCACCTCTTGTTCGGAACT |
| | | GGAACCAAGGTCACCGTGCTGACCACTACCCCAGCACCGAGGCCAC |
| | | CCACCCCGGCTCCTACCATCGCCTCCCAGCCTCTGTCCCTGCGTCCG |
| | | GAGGCATGTAGACCCGCAGCTGGTGGGGCCGTGCATACCCGGGGTC |
| | | TTGACTTCGCCTGCGATATCTACATTTGGGCCCCTCTGGCTGGTACT |
| | | TGCGGGGTCCTGCTGCTTTCACTCGTGATCACTCTTTACTGTAAGCG |
| | | CGGTCGGAAGAAGCTGCTGTACATCTTTAAGCAACCCTTCATGAGG |
| | | CCTGTGCAGACTACTCAAGAGGAGGACGGCTGTTCATGCCGGTTCC |
| | | CAGAGGAGGAGGAAGGCGGCTGCGAACTGCGCGTGAAATTCAGCC |
| | | GCAGCGCAGATGCTCCAGCCTACAAGCAGGGGCAGAACCAGCTCT |
| | | ACAACGAACTCAATCTTGGTCGGAGAGAGGAGTACGACGTGCTGG |
| | | ACAAGCGGAGAGGACGGGACCCAGAAATGGGCGGGAAGCCGCGCA |
| | | GAAAGAATCCCCAAGAGGGCCTGTACAACGAGCTCCAAAAGGATA |
| | | AGATGGCAGAAGCCTATAGCGAGATTGGTATGAAAGGGGAACGCA |
| | | GAAGAGGCAAAGGCCACGACGGACTGTACCAGGGACTCAGCACCG |
| | | CCACCAAGGACACCTATGACGCTCTTCACATGCAGGCCCTGCCGCC |
| | | TCGG |

BCMA_EBB-C1978-A4

| | | |
|---|---|---|
| BCMA_EBB- | 137 | EVQLVESGGGLVQPGGSLRLSCAASGFTFSSYAMSWVRQAPGKGLEW |
| C1978-A4- | | VSAISGSGGSTYYADSVKGRFTISRDNSKNTLYLQMNSLRAEDTAVYY |
| aa | | CAKVEGSGSLDYWGQGTLVTVSSGGGGSGGGGSGGGGSEIVMTQSPG |
| ScFv domain | | TLSLSPGERATLSCRASQSVSSAYLAWYQQKPGQPPRLLISGASTRATG |
| | | IPDRFGGSGSGTDFTLTISRLEPEDFAVYYCQHYGSSFNGSSLFTFGQGT |
| | | RLEIK |
| BCMA_EBB- | 158 | GAAGTGCAGCTCGTGGAGTCAGGAGGCGGCCTGGTCCAGCCGGGA |
| C1978-A4-nt | | GGGTCCCTTAGACTGTCATGCGCCGCAAGCGGATTCACTTTCTCCTC |
| ScFv domain | | CTATGCCATGAGCTGGGTCCGCCAAGCCCCCGGAAAGGGACTGGAA |
| | | TGGGTGTCCGCCATCTCGGGGTCTGGAGGCTCAACTTACTACGCTG |
| | | ACTCCGTGAAGGGACGGTTCACCATTAGCCGCGACAACTCCAAGAA |
| | | CACCCTCTACCTCCAAATGAACTCCCTGCGGGCCGAGGATACCGCC |
| | | GTCTACTACTGCGCCAAAGTGGAAGGTTCAGGATCGCTGGACTACT |
| | | GGGGACAGGGTACTCTCGTGACCGTGTCATCGGGCGGAGGAGGTTC |
| | | CGGCGGTGGCGGCTCCGGCGGCGGAGGGTCGGAGATCGTGATGAC |
| | | CCAGAGCCCTGGTACTCTGAGCCTTTCGCCGGGAGAAAGGGCCACC |
| | | CTGTCCTGCCGCGCTTCCCAATCCGTGTCCTCCGCGTACTTGGCGTG |
| | | GTACCAGCAGAAGCCGGGACAGCCCCCTCGGCTGCTGATCAGCGGG |
| | | GCCAGCACCCGGGCAACCGGAATCCCAGACAGATTCGGGGGTTCCG |
| | | GCAGCGGCACAGATTTCACCCTGACTATTTCGAGGTTGGAGCCCGA |
| | | GGACTTTGCGGTGTATTACTGTCAGCACTACGGGTCGTCCTTTAATG |
| | | GCTCCAGCCTGTTCACGTTCGGACAGGGGACCCGCCTGGAAATCAA |
| | | G |
| BCMA_EBB- | 179 | EVQLVESGGGLVQPGGSLRLSCAASGFTFSSYAMSWVRQAPGKGLEW |
| C1978-A4- | | VSAISGSGGSTYYADSVKGRFTISRDNSKNTLYLQMNSLRAEDTAVYY |
| aa | | CAKVEGSGSLDYWGQGTLVTVSS |
| VH | | |
| BCMA_EBB- | 200 | EIVMTQSPGTLSLSPGERATLSCRASQSVSSAYLAWYQQKPGQPPRLLI |
| C1978-A4- | | SGASTRATGIPDRFGGSGSGTDFTLTISRLEPEDFAVYYCQHYGSSFNGS |
| aa | | SLFTFGQGTRLEIK |
| VL | | |
| BCMA_EBB- | 221 | MALPVTALLLPLALLLHAARPEVQLVESGGGLVQPGGSLRLSCAASGF |
| C1978-A4- | | TFSSYAMSWVRQAPGKGLEWVSAISGSGGSTYYADSVKGRFTISRDNS |
| aa | | KNTLYLQMNSLRAEDTAVYYCAKVEGSGSLDYWGQGTLVTVSSGGG |
| Full CART | | GSGGGGSGGGGSEIVMTQSPGTLSLSPGERATLSCRASQSVSSAYLAW |
| | | YQQKPGQPPRLLISGASTRATGIPDRFGGSGSGTDFTLTISRLEPEDFAV |
| | | YYCQHYGSSFNGSSLFTFGQGTRLEIKTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYD |
| | | ALHMQALPPR |
| BCMA_EBB- | 242 | ATGGCCCTCCCTGTCACCGCCCTGCTGCTTCCGCTGGCTCTTCTGCT |
| C1978-A4-nt | | CCACGCCGCTCGGCCCGAAGTGCAGCTCGTGGAGTCAGGAGGCGGC |
| Full CART | | CTGGTCCAGCCGGGAGGGTCCCTTAGACTGTCATGCGCCGCAAGCG |
| | | GATTCACTTTCTCCTCCTATGCCATGAGCTGGGTCCGCCAAGCCCCC |
| | | GGAAAGGGACTGGAATGGGTGTCCGCCATCTCGGGGTCTGGAGGCT |
| | | CAACTTACTACGCTGACTCCGTGAAGGGACGGTTCACCATTAGCCG |
| | | CGACAACTCCAAGAACACCCTCTACCTCCAAATGAACTCCCTGCGG |
| | | GCCGAGGATACCGCCGTCTACTACTGCGCCAAAGTGGAAGGTTCAG |
| | | GATCGCTGGACTACTGGGGACAGGGTACTCTCGTGACCGTGTCATC |
| | | GGGCGGAGGAGGTTCCGGCGGTGGCGGCTCCGGCGGCGGAGGGTC |
| | | GGAGATCGTGATGACCCAGAGCCCTGGTACTCTGAGCCTTTCGCCG |
| | | GGAGAAAGGGCCACCCTGTCCTGCCGCGCTTCCCAATCCGTGTCCT |
| | | CCGCGTACTTGGCGTGGTACCAGCAGAAGCCGGGACAGCCCCCTCG |
| | | GCTGCTGATCAGCGGGGCCAGCACCCGGGCAACCGGAATCCCAGA |
| | | CAGATTCGGGGGTTCCGGCAGCGGCACAGATTTCACCCTGACTATT |
| | | TCGAGGTTGGAGCCCGAGGACTTTGCGGTGTATTACTGTCAGCACT |
| | | ACGGGTCGTCCTTTAATGGCTCCAGCCTGTTCACGTTCGGACAGGG |
| | | GACCCGCCTGGAAATCAAGACCACTACCCCAGCACCGAGGCCACCC |
| | | ACCCCGGCTCCTACCATCGCCTCCCAGCCTCTGTCCCTGCGTCCGGA |
| | | GGCATGTAGACCCGCAGCTGGTGGGGCCGTGCATACCCGGGGTCTT |
| | | GACTTCGCCTGCGATATCTACATTTGGGCCCCTCTGGCTGGTACTTG |
| | | CGGGGTCCTGCTGCTTTCACTCGTGATCACTCTTTACTGTAAGCGCG |
| | | GTCGGAAGAAGCTGCTGTACATCTTTAAGCAACCCTTCATGAGGCC |
| | | TGTGCAGACTACTCAAGAGGAGGACGGCTGTTCATGCCGGTTCCCA |
| | | GAGGAGGAGGAAGGCGGCTGCGAACTGCGCGTGAAATTCAGCCGC |
| | | AGCGCAGATGCTCCAGCCTACAAGCAGGGGCAGAACCAGCTCTAC |
| | | AACGAACTCAATCTTGGTCGGAGAGAGGAGTACGACGTGCTGGAC |
| | | AAGCGGAGAGGACGGGACCCAGAAATGGGCGGGAAGCCGCGCAG |
| | | AAAGAATCCCCAAGAGGGCCTGTACAACGAGCTCCAAAAGGATAA |
| | | GATGGCAGAAGCCTATAGCGAGATTGGTATGAAAGGGGAACGCAG |
| | | AAGAGGCAAAGGCCACGACGGACTGTACCAGGGACTCAGCACCGC |
| | | CACCAAGGACACCTATGACGCTCTTCACATGCAGGCCCTGCCGCCT |
| | | CGG |

BCMA_EBB-C1978-G1

| | | |
|---|---|---|
| BCMA_EBB- | 138 | EVQLVETGGGLVQPGGSLRLSCAASGITFSRYPMSWVRQAPGKGLEW |
| C1978-G1- | | VSGISDSGVSTYYADSAKGRFTISRDNSKNTLFLQMSSLRDEDTAVYY |
| aa | | CVTRAGSEASDIWGQGTMVTVSSGGGGSGGGGSGGGGSEIVLTQSPAT |
| ScFv domain | | LSLSPGERATLSCRASQSVSNSLAWYQQKPGQAPRLLIYDASSRATGIP |
| | | DRFSGSGSGTDFTLTISRLEPEDFAIYYCQQFGTSSGLTFGGGTKLEIK |
| BCMA_EBB | 159 | GAAGTGCAACTGGTGGAAACCGGTGGCGGCCTGGTGCAGCCTGGA |
| C1978-G1 - | | GGATCATTGAGGCTGTCATGCGCGGCCAGCGGTATTACCTTCTCCC |
| nt | | GGTACCCCATGTCCTGGGTCAGACAGGCCCCGGGGAAAGGGCTTGA |
| ScFv domain | | ATGGGTGTCCGGGATCTCGGACTCCGGTGTCAGCACTTACTACGCC |
| | | GACTCCGCCAAGGGACGCTTCACCATTTCCCGGGACAACTCGAAGA |
| | | ACACCCTGTTCCTCCAAATGAGCTCCCTCCGGGACGAGGATACTGC |
| | | AGTGTACTACTGCGTGACCCGCGCCGGGTCCGAGGCGTCTGACATT |
| | | TGGGGACAGGGCACTATGGTCACCGTGTCGTCCGGCGGAGGGGGCT |
| | | CGGGAGGCGGTGGCAGCGGAGGAGGAGGGTCCGAGATCGTGCTGA |
| | | CCCAATCCCCGGCCACCCTCTCGCTGAGCCCTGGAGAAAGGGCAAC |
| | | CTTGTCCTGTCGCGCGAGCCAGTCCGTGAGCAACTCCCTGGCCTGGT |
| | | ACCAGCAGAAGCCCGGACAGGCTCCGAGACTTCTGATCTACGACGC |
| | | TTCGAGCCGGGCCACTGGAATCCCCGACCGCTTTTCGGGGTCCGGC |
| | | TCAGGAACCGATTTCACCCTGACAATCTCACGGCTGGAGCCAGAGG |
| | | ATTTCGCCATCTATTACTGCCAGCAGTTCGGTACTTCCTCCGGCCTG |
| | | ACTTTCGGAGGCGGCACGAAGCTCGAAATCAAG |
| BCMA_EBB- | 180 | EVQLVETGGGLVQPGGSLRLSCAASGITFSRYPMSWVRQAPGKGLEW |
| C1978-G1- | | VSGISDSGVSTYYADSAKGRFTISRDNSKNTLFLQMSSLRDEDTAVYY |
| aa | | CVTRAGSEASDIWGQGTMVTVSS |
| VH | | |
| BCMA_EBB- | 201 | EIVLTQSPATLSLSPGERATLSCRASQSVSNSLAWYQQKPGQAPRLLIY |
| C1978-G1- | | DASSRATGIPDRFSGSGSGTDFTLTISRLEPEDFAIYYCQQFGTSSGLTFG |
| aa | | GGTKLEIK |
| VL | | |
| BCMA_EBB- | 222 | MALPVTALLLPLALLLHAARPEVQLVETGGGLVQPGGSLRLSCAASGI |
| C1978-G1- | | TFSRYPMSWVRQAPGKGLEWVSGISDSGVSTYYADSAKGRFTISRDNS |
| aa | | KNTLFLQMSSLRDEDTAVYYCVTRAGSEASDIWGQGTMVTVSSGGGG |
| Full CART | | SGGGGSGGGGSEIVLTQSPATLSLSPGERATLSCRASQSVSNSLAWYQQ |
| | | KPGQAPRLLIYDASSRATGIPDRFSGSGSGTDFTLTISRLEPEDFAIYYCQ |
| | | QFGTSSGLTFGGGTKLEIKTTTPAPRPPTPAPTIASQPLSLRPEACRPAAG |
| | | GAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQK |
| | | DKMAEAYSEIGMKGERRRGKGHDGLYQGLSTATKDTYDALHMQALP |
| | | PR |
| BCMA_EBB- | 243 | ATGGCCCTCCCTGTCACCGCCCTGCTGCTTCCGCTGGCTCTTCTGCT |
| C1978-G1- | | CCACGCCGCTCGGCCCGAAGTGCAACTGGTGGAAACCGGTGGCGGC |
| nt | | CTGGTGCAGCCTGGAGGATCATTGAGGCTGTCATGCGCGGCCAGCG |
| Full CART | | GTATTACCTTCTCCCGGTACCCCATGTCCTGGGTCAGACAGGCCCCG |
| | | GGGAAAGGGCTTGAATGGGTGTCCGGGATCTCGGACTCCGGTGTCA |
| | | GCACTTACTACGCCGACTCCGCCAAGGGACGCTTCACCATTTCCCG |
| | | GGACAACTCGAAGAACACCCTGTTCCTCCAAATGAGCTCCCTCCGG |
| | | GACGAGGATACTGCAGTGTACTACTGCGTGACCCGCGCCGGGTCCG |
| | | AGGCGTCTGACATTTGGGGACAGGGCACTATGGTCACCGTGTCGTC |
| | | CGGCGGAGGGGGCTCGGGAGGCGGTGGCAGCGGAGGAGGAGGGTC |
| | | CGAGATCGTGCTGACCCAATCCCCGGCCACCCTCTCGCTGAGCCCT |
| | | GGAGAAAGGGCAACCTTGTCCTGTCGCGCGAGCCAGTCCGTGAGCA |
| | | ACTCCCTGGCCTGGTACCAGCAGAAGCCCGGACAGGCTCCGAGACT |
| | | TCTGATCTACGACGCTTCGAGCCGGGCCACTGGAATCCCCGACCGC |
| | | TTTTCGGGGTCCGGCTCAGGAACCGATTTCACCCTGACAATCTCACG |
| | | GCTGGAGCCAGAGGATTTCGCCATCTATTACTGCCAGCAGTTCGGT |
| | | ACTTCCTCCGGCCTGACTTTCGGAGGCGGCACGAAGCTCGAAATCA |
| | | AGACCACTACCCCAGCACCGAGGCCACCCACCCCGGCTCCTACCAT |
| | | CGCCTCCCAGCCTCTGTCCCTGCGTCCGGAGGCATGTAGACCCGCA |
| | | GCTGGTGGGGCCGTGCATACCCGGGGTCTTGACTTCGCCTGCGATA |
| | | TCTACATTTGGGCCCCTCTGGCTGGTACTTGCGGGGTCCTGCTGCTT |
| | | TCACTCGTGATCACTCTTTACTGTAAGCGCGGTCGGAAGAAGCTGC |
| | | TGTACATCTTTAAGCAACCCTTCATGAGGCCTGTGCAGACTACTCAA |
| | | GAGGAGGACGGCTGTTCATGCCGGTTCCCAGAGGAGGAGGAAGGC |
| | | GGCTGCGAACTGCGCGTGAAATTCAGCCGCAGCGCAGATGCTCCAG |
| | | CCTACAAGCAGGGGCAGAACCAGCTCTACAACGAACTCAATCTTGG |
| | | TCGGAGAGAGGAGTACGACGTGCTGGACAAGCGGAGAGGACGGGA |
| | | CCCAGAAATGGGCGGGAAGCCGCGCAGAAAGAATCCCCAAGAGGG |
| | | CCTGTACAACGAGCTCCAAAAGGATAAGATGGCAGAAGCCTATAG |
| | | CGAGATTGGTATGAAAGGGGAACGCAGAAGAGGCAAAGGCCACGA |
| | | CGGACTGTACCAGGGACTCAGCACCGCCACCAAGGACACCTATGAC |
| | | GCTCTTCACATGCAGGCCCTGCCGCCTCGG |

BCMA_EBB-C1979-C1

| | | |
|---|---|---|
| BCMA_EBB- | 139 | QVQLVESGGGLVQPGGSLRLSCAASGFTFSSYAMSWVRQAPGKGLEW |
| C1979-C1- | | VSAISGSGGSTYYADSVKGRFTISRDNAKNSLYLQMNSLRAEDTAIYY |
| aa | | CARATYKRELRYYYGMDVWGQGTMVTVSSGGGGSGGGGSGGGGSEI |
| ScFv domain | | VMTQSPGTVSLSPGERATLSCRASQSVSSSFLAWYQQKPGQAPRLLIY |
| | | GASSRATGIPDRFSGSGSGTDFTLTISRLEPEDSAVYYCQQYHSSPSWTF |
| | | GQGTRLEIK |
| BCMA_EBB- | 160 | CAAGTGCAGCTCGTGGAATCGGGTGGCGGACTGGTGCAGCCGGGG |
| C1979-C1-nt | | GGCTCACTTAGACTGTCCTGCGCGGCCAGCGGATTCACTTTCTCCTC |
| ScFv domain | | CTACGCCATGTCCTGGGTCAGACAGGCCCCTGGAAAGGGCCTGGAA |
| | | TGGGTGTCCGCAATCAGCGGCAGCGGCGGCTCGACCTATTACGCGG |
| | | ATTCAGTGAAGGGCAGATTCACCATTTCCCGGGACAACGCCAAGAA |
| | | CTCCTTGTACCTTCAAATGAACTCCCTCCGCGCGGAAGATACCGCA |
| | | ATCTACTACTGCGCTCGGGCCACTTACAAGAGGGAACTGCGCTACT |
| | | ACTACGGGATGGACGTCTGGGGCCAGGGAACCATGGTCACCGTGTC |
| | | CAGCGGAGGAGGAGGATCGGGAGGAGGCGGTAGCGGGGGTGGAG |
| | | GGTCGGAGATCGTGATGACCCAGTCCCCCGGCACTGTGTCGCTGTC |
| | | CCCCGGCGAACGGGCCACCCTGTCATGTCGGGCCAGCCAGTCAGTG |
| | | TCGTCAAGCTTCCTCGCCTGGTACCAGCAGAAACCGGGACAAGCTC |
| | | CCCGCCTGCTGATCTACGGAGCCAGCAGCCGGGCCACCGGTATTCC |
| | | TGACCGGTTCTCCGGTTCGGGGTCCGGGACCGACTTTACTCTGACTA |
| | | TCTCTCGCCTCGAGCCAGAGGACTCCGCCGTGTATTACTGCCAGCA |
| | | GTACCACTCCTCCCCGTCCTGGACGTTCGGACAGGGCACAAGGCTG |
| | | GAGATTAAG |
| BCMA_EBB- | 181 | QVQLVESGGGLVQPGGSLRLSCAASGFTFSSYAMSWVRQAPGKGLEW |
| C1979-C1- | | VSAISGSGGSTYYADSVKGRFTISRDNAKNSLYLQMNSLRAEDTAIYY |
| aa | | CARATYKRELRYYYGMDVWGQGTMVTVSS |
| VH | | |
| BCMA_EBB- | 202 | EIVMTQSPGTVSLSPGERATLSCRASQSVSSSFLAWYQQKPGQAPRLLI |
| C1979-C1- | | YGASSRATGIPDRFSGSGSGTDFTLTISRLEPEDSAVYYCQQYHSSPSW |
| a | | TFGQGTRLEIK |
| VL | | |
| BCMA_EBB- | 223 | MALPVTALLLPLALLLHAARPQVQLVESGGGLVQPGGSLRLSCAASGF |
| C1979-C1- | | TFSSYAMSWVRQAPGKGLEWVSAISGSGGSTYYADSVKGRFTISRDNA |
| aa | | KNSLYLQMNSLRAEDTAIYYCARATYKRELRYYYGMDVWGQGTMV |
| Full CART | | TVSSGGGGSGGGGSGGGGSEIVMTQSPGTVSLSPGERATLSCRASQSVS |
| | | SSFLAWYQQKPGQAPRLLIYGASSRATGIPDRFSGSGSGTDFTLTISRLE |
| | | PEDSAVYYCQQYHSSPSWTFGQGTRLEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKN |
| | | PQEGLYNELQKDKMAEAYSEIGMKGERRRGKGHDGLYQGLSTATKD |
| | | TYDALHMQALPPR |
| BCMA_EBB- | 244 | ATGGCCCTCCCTGTCACCGCCCTGCTGCTTCCGCTGGCTCTTCTGCT |
| C1979-C1-nt | | CCACGCCGCTCGGCCCCAAGTGCAGCTCGTGGAATCGGGTGGCGGA |
| Full CART | | CTGGTGCAGCCGGGGGGCTCACTTAGACTGTCCTGCGCGGCCAGCG |
| | | GATTCACTTTCTCCTCCTACGCCATGTCCTGGGTCAGACAGGCCCCT |
| | | GGAAAGGGCCTGGAATGGGTGTCCGCAATCAGCGGCAGCGGCGGC |
| | | TCGACCTATTACGCGGATTCAGTGAAGGGCAGATTCACCATTTCCC |
| | | GGGACAACGCCAAGAACTCCTTGTACCTTCAAATGAACTCCCTCCG |
| | | CGCGGAAGATACCGCAATCTACTACTGCGCTCGGGCCACTTACAAG |
| | | AGGGAACTGCGCTACTACTACGGGATGGACGTCTGGGGCCAGGGA |
| | | ACCATGGTCACCGTGTCCAGCGGAGGAGGAGGATCGGGAGGAGGC |
| | | GGTAGCGGGGGTGGAGGGTCGGAGATCGTGATGACCCAGTCCCCC |
| | | GGCACTGTGTCGCTGTCCCCCGGCGAACGGGCCACCCTGTCATGTC |
| | | GGGCCAGCCAGTCAGTGTCGTCAAGCTTCCTCGCCTGGTACCAGCA |
| | | GAAACCGGGACAAGCTCCCCGCCTGCTGATCTACGGAGCCAGCAGC |
| | | CGGGCCACCGGTATTCCTGACCGGTTCTCCGGTTCGGGGTCCGGGA |
| | | CCGACTTTACTCTGACTATCTCTCGCCTCGAGCCAGAGGACTCCGCC |
| | | GTGTATTACTGCCAGCAGTACCACTCCTCCCCGTCCTGGACGTTCGG |
| | | ACAGGGCACAAGGCTGGAGATTAAGACCACTACCCCAGCACCGAG |
| | | GCCACCCACCCCGGCTCCTACCATCGCCTCCCAGCCTCTGTCCCTGC |
| | | GTCCGGAGGCATGTAGACCCGCAGCTGGTGGGGCCGTGCATACCCG |
| | | GGGTCTTGACTTCGCCTGCGATATCTACATTTGGGCCCCTCTGGCTG |
| | | GTACTTGCGGGGTCCTGCTGCTTTCACTCGTGATCACTCTTTACTGT |
| | | AAGCGCGGTCGGAAGAAGCTGCTGTACATCTTTAAGCAACCCTTCA |
| | | TGAGGCCTGTGCAGACTACTCAAGAGGAGGACGGCTGTTCATGCCG |
| | | GTTCCCAGAGGAGGAGGAAGGCGGCTGCGAACTGCGCGTGAAATT |
| | | CAGCCGCAGCGCAGATGCTCCAGCCTACAAGCAGGGGCAGAACCA |
| | | GCTCTACAACGAACTCAATCTTGGTCGGAGAGAGGAGTACGACGTG |
| | | CTGGACAAGCGGAGAGGACGGGACCCAGAAATGGGCGGGAAGCCG |
| | | CGCAGAAAGAATCCCCAAGAGGGCCTGTACAACGAGCTCCAAAAG |
| | | GATAAGATGGCAGAAGCCTATAGCGAGATTGGTATGAAAGGGGAA |
| | | CGCAGAAGAGGCAAAGGCCACGACGGACTGTACCAGGGACTCAGC |
| | | ACCGCCACCAAGGACACCTATGACGCTCTTCACATGCAGGCCCTGC |
| | | CGCCTCGG |

BCMA_EBB-C1978-C7

| | | |
|---|---|---|
| BCMA_EBB- | 140 | EVQLVETGGGLVQPGGSLRLSCAASGFTFSSYAMSWVRQAPGKGLEW |
| C1978-C7- | | VSAISGSGGSTYYADSVKGRFTISRDNSKNTLYLQMNTLKAEDTAVYY |
| aa | | CARATYKRELRYYYGMDVWGQGTTVTVSSGGGGSGGGGSGGGGSEI |
| ScFv domain | | VLTQSPSTLSLSPGESATLSCRASQSVSTTFLAWYQQKPGQAPRLLIYG |
| | | SSNRATGIPDRFSGSGSGTDFTLTIRRLEPEDFAVYYCQQYHSSPSWTFG |
| | | QGTKVEIK |
| BCMA_EBB- | 161 | GAGGTGCAGCTTGTGGAAACCGGTGGCGGACTGGTGCAGCCCGGA |
| C1978-C7-nt | | GGAAGCCTCAGGCTGTCCTGCGCCGCGTCCGGCTTCACCTTCTCCTC |
| ScFv domain | | GTACGCCATGTCCTGGGTCCGCCAGGCCCCCGGAAAGGGCCTGGAA |
| | | TGGGTGTCCGCCATCTCTGGAAGCGGAGGTTCCACGTACTACGCGG |
| | | ACAGCGTCAAGGGAAGGTTCACAATCTCCCGCGATAATTCGAAGAA |
| | | CACTCTGTACCTTCAAATGAACACCCTGAAGGCCGAGGACACTGCT |
| | | GTGTACTACTGCGCACGGGCCACCTACAAGAGAGAGCTCCGGTACT |
| | | ACTACGGAATGGACGTCTGGGGCCAGGGAACTACTGTGACCGTGTC |
| | | CTCGGGAGGGGGTGGCTCCGGGGGGGGCGGCTCCGGCGGAGGCGG |
| | | TTCCGAGATTGTGCTGACCCAGTCACCTTCAACTCTGTCGCTGTCCC |
| | | CGGGAGAGAGCGCTACTCTGAGCTGCCGGGCCAGCCAGTCCGTGTC |
| | | CACCACCTTCCTCGCCTGGTATCAGCAGAAGCCGGGGCAGGCACCA |
| | | CGGCTCTTGATCTACGGGTCAAGCAACAGAGCGACCGGAATTCCTG |
| | | ACCGCTTCTCGGGGAGCGGTTCAGGCACCGACTTCACCCTGACTAT |
| | | CCGGCGCCTGGAACCCGAAGATTTCGCCGTGTATTACTGTCAACAG |
| | | TACCACTCCTCGCCGTCCTGGACCTTTGGCCAAGGAACCAAAGTGG |
| | | AAATCAAG |
| BCMA_EBB- | 182 | EVQLVETGGGLVQPGGSLRLSCAASGFTFSSYAMSWVRQAPGKGLEW |
| C1978-C7- | | VSAISGSGGSTYYADSVKGRFTISRDNSKNTLYLQMNTLKAEDTAVYY |
| aa | | CARATYKRELRYYYGMDVWGQGTTVTVSS |
| VH | | |
| BCMA_EBB- | 203 | EIVLTQSPSTLSLSPGESATLSCRASQSVSTTFLAWYQQKPGQAPRLLIY |
| C1978-C7- | | GSSNRATGIPDRFSGSGSGTDFTLTIRRLEPEDFAVYYCQQYHSSPSWTF |
| aa | | GQGTKVEIK |
| VL | | |
| BCMA_EBB- | 224 | MALPVTALLLPLALLLHAARPEVQLVETGGGLVQPGGSLRLSCAASGF |
| C1978-C7- | | TFSSYAMSWVRQAPGKGLEWVSAISGSGGSTYYADSVKGRFTISRDNS |
| aa | | KNTLYLQMNTLKAEDTAVYYCARATYKRELRYYYGMDVWGQGTTV |
| Full CART | | TVSSGGGGSGGGGSGGGGSEIVLTQSPSTLSLSPGESATLSCRASQSVST |
| | | TFLAWYQQKPGQAPRLLIYGSSNRATGIPDRFSGSGSGTDFTLTIRRLEP |
| | | EDFAVYYCQQYHSSPSWTFGQGTKVEIKTTTPAPRPPTPAPTIASQPLSL |
| | | RPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDT |
| | | YDALHMQALPPR |
| BCMA_EBB- | 245 | ATGGCCCTCCCTGTCACCGCCCTGCTGCTTCCGCTGGCTCTTCTGCT |
| C1978-C7-nt | | CCACGCCGCTCGGCCCGAGGTGCAGCTTGTGGAAACCGGTGGCGGA |
| Full CART | | CTGGTGCAGCCCGGAGGAAGCCTCAGGCTGTCCTGCGCCGCGTCCG |
| | | GCTTCACCTTCTCCTCGTACGCCATGTCCTGGGTCCGCCAGGCCCCC |
| | | GGAAAGGGCCTGGAATGGGTGTCCGCCATCTCTGGAAGCGGAGGTT |
| | | CCACGTACTACGCGGACAGCGTCAAGGGAAGGTTCACAATCTCCCG |
| | | CGATAATTCGAAGAACACTCTGTACCTTCAAATGAACACCCTGAAG |
| | | GCCGAGGACACTGCTGTGTACTACTGCGCACGGGCCACCTACAAGA |
| | | GAGAGCTCCGGTACTACTACGGAATGGACGTCTGGGGCCAGGGAA |
| | | CTACTGTGACCGTGTCCTCGGGAGGGGGTGGCTCCGGGGGGGGCGG |
| | | CTCCGGCGGAGGCGGTTCCGAGATTGTGCTGACCCAGTCACCTTCA |
| | | ACTCTGTCGCTGTCCCCGGGAGAGAGCGCTACTCTGAGCTGCCGGG |
| | | CCAGCCAGTCCGTGTCCACCACCTTCCTCGCCTGGTATCAGCAGAA |
| | | GCCGGGGCAGGCACCACGGCTCTTGATCTACGGGTCAAGCAACAGA |
| | | GCGACCGGAATTCCTGACCGCTTCTCGGGGAGCGGTTCAGGCACCG |
| | | ACTTCACCCTGACTATCCGGCGCCTGGAACCCGAAGATTTCGCCGT |
| | | GTATTACTGTCAACAGTACCACTCCTCGCCGTCCTGGACCTTTGGCC |
| | | AAGGAACCAAAGTGGAAATCAAGACCACTACCCCAGCACCGAGGC |
| | | CACCCACCCCGGCTCCTACCATCGCCTCCCAGCCTCTGTCCCTGCGT |
| | | CCGGAGGCATGTAGACCCGCAGCTGGTGGGGCCGTGCATACCCGGG |
| | | GTCTTGACTTCGCCTGCGATATCTACATTTGGGCCCCTCTGGCTGGT |
| | | ACTTGCGGGGTCCTGCTGCTTTCACTCGTGATCACTCTTTACTGTAA |
| | | GCGCGGTCGGAAGAAGCTGCTGTACATCTTTAAGCAACCCTTCATG |
| | | AGGCCTGTGCAGACTACTCAAGAGGAGGACGGCTGTTCATGCCGGT |
| | | TCCCAGAGGAGGAGGAAGGCGGCTGCGAACTGCGCGTGAAATTCA |
| | | GCCGCAGCGCAGATGCTCCAGCCTACAAGCAGGGGCAGAACCAGC |
| | | TCTACAACGAACTCAATCTTGGTCGGAGAGAGGAGTACGACGTGCT |
| | | GGACAAGCGGAGAGGACGGGACCCAGAAATGGGCGGGAAGCCGC |
| | | GCAGAAAGAATCCCCAAGAGGGCCTGTACAACGAGCTCCAAAAGG |
| | | ATAAGATGGCAGAAGCCTATAGCGAGATTGGTATGAAAGGGGAAC |
| | | GCAGAAGAGGCAAAGGCCACGACGGACTGTACCAGGGACTCAGCA |
| | | CCGCCACCAAGGACACCTATGACGCTCTTCACATGCAGGCCCTGCC |
| | | GCCTCGG |

BCMA_EBB-C1978-D10

| | | |
|---|---|---|
| BCMA_EBB- | 141 | EVQLVETGGGLVQPGRSLRLSCAASGFTFDDYAMHWVRQAPGKGLE |
| C1978-D10- | | WVSGISWNSGSIGYADSVKGRFTISRDNAKNSLYLQMNSLRDEDTAVY |
| aa | | YCARVGKAVPDVWGQGTTVTVSSGGGGSGGGGSGGGGSDIVMTQTP |
| ScFv domain | | SSLSASVGDRVTITCRASQSISSYLNWYQQKPGKAPKLLIYAASSLQSG |
| | | VPSRFSGSGSGTDFTLTISSLQPEDFATYYCQQSYSTPYSFGQGTRLEIK |
| BCMA_EBB- | 162 | GAAGTGCAGCTCGTGGAAACTGGAGGTGGACTCGTGCAGCCTGGAC |
| C1978-D10- | | GGTCGCTGCGGCTGAGCTGCGCTGCATCCGGCTTCACCTTCGACGA |
| nt | | TTATGCCATGCACTGGGTCAGACAGGCGCCAGGGAAGGGACTTGAG |
| ScFv domain | | TGGGTGTCCGGTATCAGCTGGAATAGCGGCTCAATCGGATACGCGG |
| | | ACTCCGTGAAGGGAAGGTTCACCATTTCCCGCGACAACGCCAAGAA |
| | | CTCCCTGTACTTGCAAATGAACAGCCTCCGGGATGAGGACACTGCC |
| | | GTGTACTACTGCGCCCGCGTCGGAAAAGCTGTGCCCGACGTCTGGG |
| | | GCCAGGGAACCACTGTGACCGTGTCCAGCGGCGGGGGTGGATCGG |
| | | GCGGTGGAGGGTCCGGTGGAGGGGGCTCAGATATTGTGATGACCCA |
| | | GACCCCCTCGTCCCTGTCCGCCTCGGTCGGCGACCGCGTGACTATCA |
| | | CATGTAGAGCCTCGCAGAGCATCTCCAGCTACCTGAACTGGTATCA |
| | | GCAGAAGCCGGGGAAGGCCCCGAAGCTCCTGATCTACGCGGCATC |
| | | ATCACTGCAATCGGGAGTGCCGAGCCGGTTTTCCGGGTCCGGCTCC |
| | | GGCACCGACTTCACGCTGACCATTTCTTCCCTGCAACCCGAGGACTT |
| | | CGCCACTTACTACTGCCAGCAGTCCTACTCCACCCCTTACTCCTTCG |
| | | GCCAAGGAACCAGGCTGGAAATCAAG |
| BCMA_EBB- | 183 | EVQLVETGGGLVQPGRSLRLSCAASGFTFDDYAMHWVRQAPGKGLE |
| C1978-D10- | | WVSGISWNSGSIGYADSVKGRFTISRDNAKNSLYLQMNSLRDEDTAVY |
| aa | | YCARVGKAVPDVWGQGTTVTVSS |
| VH | | |
| BCMA_EBB- | 204 | DIVMTQTPSSLSASVGDRVTITCRASQSISSYLNWYQQKPGKAPKLLIY |
| C1978-D10- | | AASSLQSGVPSRFSGSGSGTDFTLTISSLQPEDFATYYCQQSYSTPYSFG |
| aa | | QGTRLEIK |
| VL | | |
| BCMA_EBB- | 225 | MALPVTALLLPLALLLHAARPEVQLVETGGGLVQPGRSLRLSCAASGF |
| C1978-D10- | | TFDDYAMHWVRQAPGKGLEWVSGISWNSGSIGYADSVKGRFTISRDN |
| aa | | AKNSLYLQMNSLRDEDTAVYYCARVGKAVPDVWGQGTTVTVSSGGG |
| Full CART | | GSGGGGSGGGGSDIVMTQTPSSLSASVGDRVTITCRASQSISSYLNWYQ |
| | | QKPGKAPKLLIYAASSLQSGVPSRFSGSGSGTDFTLTISSLQPEDFATYY |
| | | CQQSYSTPYSFGQGTRLEIKTTTPAPRPPTPAPTIASQPLSLRPEACRPAA |
| | | GGAVHTRGLDFACDIYIWAPLAGTCGVLLLSLVITLYCKRGRKKLLYIF |
| | | KQPFMRPVQTTQEEDGCSCRFPEEEEGGCELRVKFSRSADAPAYKQGQ |
| | | NQLYNELNLGRREEYDVLDKRRGRDPEMGGKPRRKNPQEGLYNELQ |
| | | KDKMAEAYSEIGMKGERRRGKGHDGLYQGLSTATKDTYDALHMQAL |
| | | PPR |
| BCMA_EBB- | 246 | ATGGCCCTCCCTGTCACCGCCCTGCTGCTTCCGCTGGCTCTTCTGCT |
| C1978-D10- | | CCACGCCGCTCGGCCCGAAGTGCAGCTCGTGGAAACTGGAGGTGGA |
| nt | | CTCGTGCAGCCTGGACGGTCGCTGCGGCTGAGCTGCGCTGCATCCG |
| Full CART | | GCTTCACCTTCGACGATTATGCCATGCACTGGGTCAGACAGGCGCC |
| | | AGGGAAGGGACTTGAGTGGGTGTCCGGTATCAGCTGGAATAGCGG |
| | | CTCAATCGGATACGCGGACTCCGTGAAGGGAAGGTTCACCATTTCC |
| | | CGCGACAACGCCAAGAACTCCCTGTACTTGCAAATGAACAGCCTCC |
| | | GGGATGAGGACACTGCCGTGTACTACTGCGCCCGCGTCGGAAAAGC |
| | | TGTGCCCGACGTCTGGGGCCAGGGAACCACTGTGACCGTGTCCAGC |
| | | GGCGGGGGTGGATCGGGCGGTGGAGGGTCCGGTGGAGGGGGCTCA |
| | | GATATTGTGATGACCCAGACCCCCTCGTCCCTGTCCGCCTCGGTCGG |
| | | CGACCGCGTGACTATCACATGTAGAGCCTCGCAGAGCATCTCCAGC |
| | | TACCTGAACTGGTATCAGCAGAAGCCGGGGAAGGCCCCGAAGCTCC |
| | | TGATCTACGCGGCATCATCACTGCAATCGGGAGTGCCGAGCCGGTT |
| | | TTCCGGGTCCGGCTCCGGCACCGACTTCACGCTGACCATTTCTTCCC |
| | | TGCAACCCGAGGACTTCGCCACTTACTACTGCCAGCAGTCCTACTCC |
| | | ACCCCTTACTCCTTCGGCCAAGGAACCAGGCTGGAAATCAAGACCA |
| | | CTACCCCAGCACCGAGGCCACCCACCCCGGCTCCTACCATCGCCTC |
| | | CCAGCCTCTGTCCCTGCGTCCGGAGGCATGTAGACCCGCAGCTGGT |
| | | GGGGCCGTGCATACCCGGGGTCTTGACTTCGCCTGCGATATCTACA |
| | | TTTGGGCCCCTCTGGCTGGTACTTGCGGGGTCCTGCTGCTTTCACTC |
| | | GTGATCACTCTTTACTGTAAGCGCGGTCGGAAGAAGCTGCTGTACA |
| | | TCTTTAAGCAACCCTTCATGAGGCCTGTGCAGACTACTCAAGAGGA |
| | | GGACGGCTGTTCATGCCGGTTCCCAGAGGAGGAGGAAGGCGGCTG |
| | | CGAACTGCGCGTGAAATTCAGCCGCAGCGCAGATGCTCCAGCCTAC |
| | | AAGCAGGGGCAGAACCAGCTCTACAACGAACTCAATCTTGGTCGGA |
| | | GAGAGGAGTACGACGTGCTGGACAAGCGGAGAGGACGGGACCCAG |
| | | AAATGGGCGGGAAGCCGCGCAGAAAGAATCCCCAAGAGGGCCTGT |
| | | ACAACGAGCTCCAAAAGGATAAGATGGCAGAAGCCTATAGCGAGA |
| | | TTGGTATGAAAGGGGAACGCAGAAGAGGCAAAGGCCACGACGGAC |
| | | TGTACCAGGGACTCAGCACCGCCACCAAGGACACCTATGACGCTCT |
| | | TCACATGCAGGCCCTGCCGCCTCGG |

BCMA_EBB-C1979-C12

| | | |
|---|---|---|
| BCMA_EBB- | 142 | EVQLVESGGGLVQPGRSLRLSCTASGFTFDDYAMHWVRQRPGKGLE |
| C1979-C12- | | WVASINWKGNSLAYGDSVKGRFAISRDNAKNTVFLQMNSLRTEDTAV |
| aa | | YYCASHQGVAYYNYAMDVWGRGTLVTVSSGGGGSGGGGSGGGGSEI |
| ScFv domain | | VLTQSPGTLSLSPGERATLSCRATQSIGSSFLAWYQQRPGQAPRLLIYG |
| | | ASQRATGIPDRFSGRGSGTDFTLTISRVEPEDSAVYYCQHYESSPSWTF |
| | | GQGTKVEIK |
| BCMA_EBB- | 163 | GAAGTGCAGCTCGTGGAGAGCGGGGGAGGATTGGTGCAGCCCGGA |
| C1979-C12- | | AGGTCCCTGCGGCTCTCCTGCACTGCGTCTGGCTTCACCTTCGACGA |
| nt | | CTACGCGATGCACTGGGTCAGACAGCGCCCGGGAAAGGGCCTGGA |
| ScFv domain | | ATGGGTCGCCTCAATCAACTGGAAGGGAAACTCCCTGGCCTATGGC |
| | | GACAGCGTGAAGGGCCGCTTCGCCATTTCGCGCGACAACGCCAAGA |
| | | ACACCGTGTTTCTGCAAATGAATTCCCTGCGGACCGAGGATACCGC |
| | | TGTGTACTACTGCGCCAGCCACCAGGGCGTGGCATACTATAACTAC |
| | | GCCATGGACGTGTGGGGAAGAGGGACGCTCGTCACCGTGTCCTCCG |
| | | GGGGCGGTGGATCGGGTGGAGGAGGAAGCGGTGGCGGGGGCAGCG |
| | | AAATCGTGCTGACTCAGAGCCCGGGAACTCTTTCACTGTCCCCGGG |
| | | AGAACGGGCCACTCTCTCGTGCCGGGCCACCCAGTCCATCGGCTCC |
| | | TCCTTCCTTGCCTGGTACCAGCAGAGGCCAGGACAGGCGCCCCGCC |
| | | TGCTGATCTACGGTGCTTCCCAACGCGCCACTGGCATTCCTGACCGG |
| | | TTCAGCGGCAGAGGGTCGGGAACCGATTTCACACTGACCATTTCCC |
| | | GGGTGGAGCCCGAAGATTCGGCAGTCTACTACTGTCAGCATTACGA |
| | | GTCCTCCCCTTCATGGACCTTCGGTCAAGGGACCAAAGTGGAGATC |
| | | AAG |
| BCMA_EBB- | 184 | EVQLVESGGGLVQPGRSLRLSCTASGFTFDDYAMHWVRQRPGKGLE |
| C1979-C12- | | WVASINWKGNSLAYGDSVKGRFAISRDNAKNTVFLQMNSLRTEDTAV |
| aa | | YYCASHQGVAYYNYAMDVWGRGTLVTVSS |
| VH | | |
| BCMA_EBB- | 205 | EIVLTQSPGTLSLSPGERATLSCRATQSIGSSFLAWYQQRPGQAPRLLIY |
| C1979-C12- | | GASQRATGIPDRFSGRGSGTDFTLTISRVEPEDSAVYYCQHYESSPSWT |
| aa | | FGQGTKVEIK |
| VL | | |
| BCMA_EBB- | 226 | MALPVTALLLPLALLLHAARPEVQLVESGGGLVQPGRSLRLSCTASGF |
| C1979-C12- | | TFDDYAMHWVRQRPGKGLEWVASINWKGNSLAYGDSVKGRFAISRD |
| aa | | NAKNTVFLQMNSLRTEDTAVYYCASHQGVAYYNYAMDVWGRGTLV |
| Full CART | | TVSSGGGGSGGGGSGGGGSEIVLTQSPGTLSLSPGERATLSCRATQSIGS |
| | | SFLAWYQQRPGQAPRLLIYGASQRATGIPDRFSGRGSGTDFTLTISRVE |
| | | PEDSAVYYCQHYESSPSWTFGQGTKVEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKN |
| | | PQEGLYNELQKDKMAEAYSEIGMKGERRRGKGHDGLYQGLSTATKD |
| | | TYDALHMQALPPR |
| BCMA_EBB- | 247 | ATGGCCCTCCCTGTCACCGCCCTGCTGCTTCCGCTGGCTCTTCTGCT |
| C1979-C12- | | CCACGCCGCTCGGCCCGAAGTGCAGCTCGTGGAGAGCGGGGGAGG |
| nt | | ATTGGTGCAGCCCGGAAGGTCCCTGCGGCTCTCCTGCACTGCGTCT |
| Full CART | | GGCTTCACCTTCGACGACTACGCGATGCACTGGGTCAGACAGCGCC |
| | | CGGGAAAGGGCCTGGAATGGGTCGCCTCAATCAACTGGAAGGGAA |
| | | ACTCCCTGGCCTATGGCGACAGCGTGAAGGGCCGCTTCGCCATTTC |
| | | GCGCGACAACGCCAAGAACACCGTGTTTCTGCAAATGAATTCCCTG |
| | | CGGACCGAGGATACCGCTGTGTACTACTGCGCCAGCCACCAGGGCG |
| | | TGGCATACTATAACTACGCCATGGACGTGTGGGGAAGAGGGACGCT |
| | | CGTCACCGTGTCCTCCGGGGGCGGTGGATCGGGTGGAGGAGGAAG |
| | | CGGTGGCGGGGGCAGCGAAATCGTGCTGACTCAGAGCCCGGGAAC |
| | | TCTTTCACTGTCCCCGGGAGAACGGGCCACTCTCTCGTGCCGGGCC |
| | | ACCCAGTCCATCGGCTCCTCCTTCCTTGCCTGGTACCAGCAGAGGCC |
| | | AGGACAGGCGCCCCGCCTGCTGATCTACGGTGCTTCCCAACGCGCC |
| | | ACTGGCATTCCTGACCGGTTCAGCGGCAGAGGGTCGGGAACCGATT |
| | | TCACACTGACCATTICCCGGGTGGAGCCCGAAGATTCGGCAGTCTA |
| | | CTACTGTCAGCATTACGAGTCCTCCCCTTCATGGACCTTCGGTCAAG |
| | | GGACCAAAGTGGAGATCAAGACCACTACCCCAGCACCGAGGCCAC |
| | | CCACCCCGGCTCCTACCATCGCCTCCCAGCCTCTGTCCCTGCGTCCG |
| | | GAGGCATGTAGACCCGCAGCTGGTGGGGCCGTGCATACCCGGGGTC |
| | | TTGACTTCGCCTGCGATATCTACATTTGGGCCCCTCTGGCTGGTACT |
| | | TGCGGGGTCCTGCTGCTTTCACTCGTGATCACTCTTTACTGTAAGCG |
| | | CGGTCGGAAGAAGCTGCTGTACATCTTTAAGCAACCCTTCATGAGG |
| | | CCTGTGCAGACTACTCAAGAGGAGGACGGCTGTTCATGCCGGTTCC |
| | | CAGAGGAGGAGGAAGGCGGCTGCGAACTGCGCGTGAAATTCAGCC |
| | | GCAGCGCAGATGCTCCAGCCTACAAGCAGGGGCAGAACCAGCTCT |
| | | ACAACGAACTCAATCTTGGTCGGAGAGAGGAGTACGACGTGCTGG |
| | | ACAAGCGGAGAGGACGGGACCCAGAAATGGGCGGGAAGCCGCGCA |
| | | GAAAGAATCCCCAAGAGGGCCTGTACAACGAGCTCCAAAAGGATA |
| | | AGATGGCAGAAGCCTATAGCGAGATTGGTATGAAAGGGGAACGCA |
| | | GAAGAGGCAAAGGCCACGACGGACTGTACCAGGGACTCAGCACCG |
| | | CCACCAAGGACACCTATGACGCTCTTCACATGCAGGCCCTGCCGCC |
| | | TCGG |

BCMA_EBB-C1980-G4

| | | |
|---|---|---|
| BCMA_EBB- | 143 | EVQLVESGGGLVQPGGSLRLSCAASGFTFSSYAMSWVRQAPGKGLEW |
| C1980-G4-aa | | VSAISGSGGSTYYADSVKGRFTISRDNSKNTLYLQMNSLRAEDTAVYY |
| ScFv domain | | CAKVVRDGMDVWGQGTTVTVSSGGGGSGGGGSGGGGSEIVLTQSPA |
| | | TLSLSPGERATLSCRASQSVSSSYLAWYQQKPGQAPRLLIYGASSRATG |
| | | IPDRFSGNGSGTDFTLTISRLEPEDFAVYYCQQYGSPPRFTFGPGTKVDI |
| | | K |
| BCMA_EBB- | 2018 | GAGGTGCAGTTGGTCGAAAGCGGGGGCGGGCTTGTGCAGCCTGGC |
| C1980-G4-nt | | GGATCACTGCGGCTGTCCTGCGCGGCATCAGGCTTCACGTTTTCTTC |
| ScFv domain | | CTACGCCATGTCCTGGGTGCGCCAGGCCCCTGGAAAGGGACTGGAA |
| | | TGGGTGTCCGCGATTTCGGGGTCCGGCGGGAGCACCTACTACGCCG |
| | | ATTCCGTGAAGGGCCGCTTCACTATCTCGCGGGACAACTCCAAGAA |
| | | CACCCTCTACCTCCAAATGAATAGCCTGCGGGCCGAGGATACCGCC |
| | | GTCTACTATTGCGCTAAGGTCGTGCGCGACGGAATGGACGTGTGGG |
| | | GACAGGGTACCACCGTGACAGTGTCCTCGGGGGGAGGCGGTAGCG |
| | | GCGGAGGAGGAAGCGGTGGTGGAGGTTCCGAGATTGTGCTGACTC |
| | | AATCACCCGCGACCCTGAGCCTGTCCCCCGGCGAAAGGGCCACTCT |
| | | GTCCTGTCGGGCCAGCCAATCAGTCTCCTCCTCGTACCTGGCCTGGT |
| | | ACCAGCAGAAGCCAGGACAGGCTCCGAGACTCCTTATCTATGGCGC |
| | | ATCCTCCCGCGCCACCGGAATCCCGGATAGGTTCTCGGGAAACGGA |
| | | TCGGGGACCGACTTCACTCTCACCATCTCCCGGCTGGAACCGGAGG |
| | | ACTTCGCCGTGTACTACTGCCAGCAGTACGGCAGCCCGCCTAGATT |
| | | CACTTTCGGCCCCGGCACCAAAGTGGACATCAAG |
| BCMA_EBB- | 185 | EVQLVESGGGLVQPGGSLRLSCAASGFTFSSYAMSWVRQAPGKGLEW |
| C1980-G4-aa | | VSAISGSGGSTYYADSVKGRFTISRDNSKNTLYLQMNSLRAEDTAVYY |
| VH | | CAKVVRDGMDVWGQGTTVTVSS |
| BCMA_EBB- | 206 | EIVLTQSPATLSLSPGERATLSCRASQSVSSSYLAWYQQKPGQAPRLLI |
| C1980-G4-aa | | YGASSRATGIPDRFSGNGSGTDFTLTISRLEPEDFAVYYCQQYGSPPRFT |
| VL | | FGPGTKVDIK |
| BCMA_EBB- | 227 | MALPVTALLLPLALLLHAARPEVQLVESGGGLVQPGGSLRLSCAASGF |
| C1980-G4-aa | | TFSSYAMSWVRQAPGKGLEWVSAISGSGGSTYYADSVKGRFTISRDNS |
| Full CART | | KNTLYLQMNSLRAEDTAVYYCAKVVRDGMDVWGQGTTVTVSSGGG |
| | | GSGGGGSGGGGSEIVLTQSPATLSLSPGERATLSCRASQSVSSSYLAWY |
| | | QQKPGQAPRLLIYGASSRATGIPDRFSGNGSGTDFTLTISRLEPEDFAVY |
| | | YCQQYGSPPRFTFGPGTKVDIKTTTPAPRPPTPAPTIASQPLSLRPEACRP |
| | | AAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| BCMA_EBB- | 248 | ATGGCCCTCCCTGTCACCGCCCTGCTGCTTCCGCTGGCTCTTCTGCT |
| C1980-G4-nt | | CCACGCCGCTCGGCCCGAGGTGCAGTTGGTCGAAAGCGGGGGCGG |
| Full CART | | GCTTGTGCAGCCTGGCGGATCACTGCGGCTGTCCTGCGCGGCATCA |
| | | GGCTTCACGTTTTCTTCCTACGCCATGTCCTGGGTGCGCCAGGCCCC |
| | | TGGAAAGGGACTGGAATGGGTGTCCGCGATTTCGGGGTCCGGCGGG |
| | | AGCACCTACTACGCCGATTCCGTGAAGGGCCGCTTCACTATCTCGC |
| | | GGGACAACTCCAAGAACACCCTCTACCTCCAAATGAATAGCCTGCG |
| | | GGCCGAGGATACCGCCGTCTACTATTGCGCTAAGGTCGTGCGCGAC |
| | | GGAATGGACGTGTGGGGACAGGGTACCACCGTGACAGTGTCCTCGG |
| | | GGGGAGGCGGTAGCGGCGGAGGAGGAAGCGGTGGTGGAGGTTCCG |
| | | AGATTGTGCTGACTCAATCACCCGCGACCCTGAGCCTGTCCCCCGG |
| | | CGAAAGGGCCACTCTGTCCTGTCGGGCCAGCCAATCAGTCTCCTCC |
| | | TCGTACCTGGCCTGGTACCAGCAGAAGCCAGGACAGGCTCCGAGAC |
| | | TCCTTATCTATGGCGCATCCTCCCGCGCCACCGGAATCCCGGATAG |
| | | GTTCTCGGGAAACGGATCGGGGACCGACTTCACTCTCACCATCTCC |
| | | CGGCTGGAACCGGAGGACTTCGCCGTGTACTACTGCCAGCAGTACG |
| | | GCAGCCCGCCTAGATTCACTTTCGGCCCCGGCACCAAAGTGGACAT |
| | | CAAGACCACTACCCCAGCACCGAGGCCACCCACCCCGGCTCCTACC |
| | | ATCGCCTCCCAGCCTCTGTCCCTGCGTCCGGAGGCATGTAGACCCG |
| | | CAGCTGGTGGGGCCGTGCATACCCGGGGTCTTGACTTCGCCTGCGA |
| | | TATCTACATTTGGGCCCCTCTGGCTGGTACTTGCGGGGTCCTGCTGC |
| | | TTTCACTCGTGATCACTCTTTACTGTAAGCGCGGTCGGAAGAAGCTG |
| | | CTGTACATCTTTAAGCAACCCTTCATGAGGCCTGTGCAGACTACTCA |
| | | AGAGGAGGACGGCTGTTCATGCCGGTTCCCAGAGGAGGAGGAAGG |
| | | CGGCTGCGAACTGCGCGTGAAATTCAGCCGCAGCGCAGATGCTCCA |
| | | GCCTACAAGCAGGGGCAGAACCAGCTCTACAACGAACTCAATCTTG |
| | | GTCGGAGAGAGGAGTACGACGTGCTGGACAAGCGGAGAGGACGGG |
| | | ACCCAGAAATGGGCGGGAAGCCGCGCAGAAAGAATCCCCAAGAGG |
| | | GCCTGTACAACGAGCTCCAAAAGGATAAGATGGCAGAAGCCTATA |
| | | GCGAGATTGGTATGAAAGGGGAACGCAGAAGAGGCAAAGGCCACG |
| | | ACGGACTGTACCAGGGACTCAGCACCGCCACCAAGGACACCTATGA |
| | | CGCTCTTCACATGCAGGCCCTGCCGCCTCGG |

BCMA_EBB-C1980-D2

| | | |
|---|---|---|
| BCMA_EBB- | 144 | EVQLLESGGGLVQPGGSLRLSCAASGFTFSSYAMSWVRQAPGKGLEW |
| C1980-D2-aa | | VSAISGSGGSTYYADSVKGRFTISRDNSKNTLYLQMNSLRAEDTAVYY |
| ScFv domain | | CAKIPQTGTFDYWGQGTLVTVSSGGGGSGGGGSGGGGSEIVLTQSPGT |
| | | LSLSPGERATLSCRASQSVSSSYLAWYQQRPGQAPRLLIYGASSRATGI |
| | | PDRFSGSGSGTDFTLTISRLEPEDFAVYYCQHYGSSPSWTFGQGTRLEIK |
| BCMA_EBB- | 165 | GAAGTGCAGCTGCTGGAGTCCGGCGGTGGATTGGTGCAACCGGGG |
| C1980-D2-nt | | GGATCGCTCAGACTGTCCTGTGCGGCGTCAGGCTTCACCTTCTCGAG |
| ScFv domain | | CTACGCCATGTCATGGGTCAGACAGGCCCCTGGAAAGGGTCTGGAA |
| | | TGGGTGTCCGCCATTTCCGGGAGCGGGGGATCTACATACTACGCCG |
| | | ATAGCGTGAAGGGCCGCTTCACCATTTCCCGGGACAACTCCAAGAA |
| | | CACTCTCTATCTGCAAATGAACTCCCTCCGCGCTGAGGACACTGCC |
| | | GTGTACTACTGCGCCAAAATCCCTCAGACCGGCACCTTCGACTACT |
| | | GGGGACAGGGGACTCTGGTCACCGTCAGCAGCGGTGGCGGAGGTT |
| | | CGGGGGGAGGAGGAAGCGGCGGCGGAGGGTCCGAGATTGTGCTGA |
| | | CCCAGTCACCCGGCACTTTGTCCCTGTCGCCTGGAGAAAGGGCCAC |
| | | CCTTTCCTGCCGGGCATCCCAATCCGTGTCCTCCTCGTACCTGGCCT |
| | | GGTACCAGCAGAGGCCCGGACAGGCCCCACGGCTTCTGATCTACGG |
| | | AGCAAGCAGCCGCGCGACCGGTATCCCGGACCGGTTTTCGGGCTCG |
| | | GGCTCAGGAACTGACTTCACCCTCACCATCTCCCGCCTGGAACCCG |
| | | AAGATTTCGCTGTGTATTACTGCCAGCACTACGGCAGCTCCCCGTCC |
| | | TGGACGTTCGGCCAGGGAACTCGGCTGGAGATCAAG |
| BCMA_EBB- | 186 | EVQLLESGGGLVQPGGSLRLSCAASGFTFSSYAMSWVRQAPGKGLEW |
| C1980-D2-aa | | VSAISGSGGSTYYADSVKGRFTISRDNSKNTLYLQMNSLRAEDTAVYY |
| VH | | CAKIPQTGTFDYWGQGTLVTVSS |
| BCMA_EBB- | 207 | EIVLTQSPGTLSLSPGERATLSCRASQSVSSSYLAWYQQRPGQAPRLLIY |
| C1980-D2-aa | | GASSRATGIPDRFSGSGSGTDFTLTISRLEPEDFAVYYCQHYGSSPSWTF |
| VL | | GQGTRLEIK |
| BCMA_EBB- | 228 | MALPVTALLLPLALLLHAARPEVQLLESGGGLVQPGGSLRLSCAASGF |
| C1980-D2-aa | | TFSSYAMSWVRQAPGKGLEWVSAISGSGGSTYYADSVKGRFTISRDNS |
| Full CART | | KNTLYLQMNSLRAEDTAVYYCAKIPQTGTFDYWGQGTLVTVSSGGGG |
| | | SGGGGSGGGGSEIVLTQSPGTLSLSPGERATLSCRASQSVSSSYLAWYQ |
| | | QRPGQAPRLLIYGASSRATGIPDRFSGSGSGTDFTLTISRLEPEDFAVYY |
| | | CQHYGSSPSWTFGQGTRLEIKTTTPAPRPPTPAPTIASQPLSLRPEACRP |
| | | AAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| BCMA_EBB- | 249 | ATGGCCCTCCCTGTCACCGCCCTGCTGCTTCCGCTGGCTCTTCTGCT |
| C1980-D2-nt | | CCACGCCGCTCGGCCCGAAGTGCAGCTGCTGGAGTCCGGCGGTGGA |
| Full CART | | TTGGTGCAACCGGGGGGATCGCTCAGACTGTCCTGTGCGGCGTCAG |
| | | GCTTCACCTTCTCGAGCTACGCCATGTCATGGGTCAGACAGGCCCCT |
| | | GGAAAGGGTCTGGAATGGGTGTCCGCCATTTCCGGGAGCGGGGGAT |
| | | CTACATACTACGCCGATAGCGTGAAGGGCCGCTTCACCATTTCCCG |
| | | GGACAACTCCAAGAACACTCTCTATCTGCAAATGAACTCCCTCCGC |
| | | GCTGAGGACACTGCCGTGTACTACTGCGCCAAAATCCCTCAGACCG |
| | | GCACCTTCGACTACTGGGGACAGGGGACTCTGGTCACCGTCAGCAG |
| | | CGGTGGCGGAGGTTCGGGGGGAGGAGGAAGCGGCGGCGGAGGGTC |
| | | CGAGATTGTGCTGACCCAGTCACCCGGCACTTTGTCCCTGTCGCCTG |
| | | GAGAAAGGGCCACCCTTTCCTGCCGGGCATCCCAATCCGTGTCCTC |
| | | CTCGTACCTGGCCTGGTACCAGCAGAGGCCCGGACAGGCCCCACGG |
| | | CTTCTGATCTACGGAGCAAGCAGCCGCGCGACCGGTATCCCGGACC |
| | | GGTTTTCGGGCTCGGGCTCAGGAACTGACTTCACCCTCACCATCTCC |
| | | CGCCTGGAACCCGAAGATTTCGCTGTGTATTACTGCCAGCACTACG |
| | | GCAGCTCCCCGTCCTGGACGTTCGGCCAGGGAACTCGGCTGGAGAT |
| | | CAAGACCACTACCCCAGCACCGAGGCCACCCACCCCGGCTCCTACC |
| | | ATCGCCTCCCAGCCTCTGTCCCTGCGTCCGGAGGCATGTAGACCCG |
| | | CAGCTGGTGGGGCCGTGCATACCCGGGGTCTTGACTTCGCCTGCGA |
| | | TATCTACATTTGGGCCCCTCTGGCTGGTACTTGCGGGGTCCTGCTGC |
| | | TTTCACTCGTGATCACTCTTTACTGTAAGCGCGGTCGGAAGAAGCTG |
| | | CTGTACATCTTTAAGCAACCCTTCATGAGGCCTGTGCAGACTACTCA |
| | | AGAGGAGGACGGCTGTTCATGCCGGTTCCCAGAGGAGGAGGAAGG |
| | | CGGCTGCGAACTGCGCGTGAAATTCAGCCGCAGCGCAGATGCTCCA |
| | | GCCTACAAGCAGGGGCAGAACCAGCTCTACAACGAACTCAATCTTG |
| | | GTCGGAGAGAGGAGTACGACGTGCTGGACAAGCGGAGAGGACGGG |
| | | ACCCAGAAATGGGCGGGAAGCCGCGCAGAAAGAATCCCCAAGAGG |
| | | GCCTGTACAACGAGCTCCAAAAGGATAAGATGGCAGAAGCCTATA |
| | | GCGAGATTGGTATGAAAGGGGAACGCAGAAGAGGCAAAGGCCACG |
| | | ACGGACTGTACCAGGGACTCAGCACCGCCACCAAGGACACCTATGA |
| | | CGCTCTTCACATGCAGGCCCTGCCGCCTCGG |

BCMA_EBB-C1978-A10

| | | |
|---|---|---|
| BCMA_EBB- | 145 | EVQLVETGGGLVQPGGSLRLSCAASGFTFSSYAMSWVRQAPGKGLEW |
| C1978-A10- | | VSAISGSGGSTYYADSVKGRFTMSRENDKNSVFLQMNSLRVEDTGVY |
| aa | | YCARANYKRELRYYYGMDVWGQGTMVTVSSGGGGSGGGGSGGGGS |
| ScFv domain | | EIVMTQSPGTLSLSPGESATLSCRASQRVASNYLAWYQHKPGQAPSLLI |
| | | SGASSRATGVPDRFSGSGSGTDFTLAISRLEPEDSAVYYCQHYDSSPSW |
| | | TFGQGTKVEIK |
| BCMA_EBB- | 166 | GAAGTGCAACTGGTGGAAACCGGTGGAGGACTCGTGCAGCCTGGC |
| C1978-A10- | | GGCAGCCTCCGGCTGAGCTGCGCCGCTTCGGGATTCACCTTTTCCTC |
| nt | | CTACGCGATGTCTTGGGTCAGACAGGCCCCCGGAAAGGGGCTGGAA |
| ScFv domain | | TGGGTGTCAGCCATCTCCGGCTCCGGCGGATCAACGTACTACGCCG |
| | | ACTCCGTGAAAGGCCGGTTCACCATGTCGCGCGAGAATGACAAGAA |
| | | CTCCGTGTTCCTGCAAATGAACTCCCTGAGGGTGGAGGACACCGGA |
| | | GTGTACTATTGTGCGCGCGCCAACTACAAGAGAGAGCTGCGGTACT |
| | | ACTACGGAATGGACGTCTGGGGACAGGGAACTATGGTGACCGTGTC |
| | | ATCCGGTGGAGGGGGAAGCGGCGGTGGAGGCAGCGGGGGCGGGGG |
| | | TTCAGAAATTGTCATGACCCAGTCCCCGGGAACTCTTTCCCTCTCCC |
| | | CCGGGGAATCCGCGACTTTGTCCTGCCGGGCCAGCCAGCGCGTGGC |
| | | CTCGAACTACCTCGCATGGTACCAGCATAAGCCAGGCCAAGCCCCT |
| | | TCCCTGCTGATTTCCGGGGCTAGCAGCCGCGCCACTGGCGTGCCGG |
| | | ATAGGTTCTCGGGAAGCGGCTCGGGTACCGATTTCACCCTGGCAAT |
| | | CTCGCGGCTGGAACCGGAGGATTCGGCCGTGTACTACTGCCAGCAC |
| | | TATGACTCATCCCCCTCCTGGACATTCGGACAGGGCACCAAGGTCG |
| | | AGATCAAG |
| BCMA_EBB- | 187 | EVQLVETGGGLVQPGGSLRLSCAASGFTFSSYAMSWVRQAPGKGLEW |
| C1978-A10- | | VSAISGSGGSTYYADSVKGRFTMSRENDKNSVFLQMNSLRVEDTGVY |
| aa | | YCARANYKRELRYYYGMDVWGQGTMVTVSS |
| VH | | |
| BCMA_EBB- | 208 | EIVMTQSPGTLSLSPGESATLSCRASQRVASNYLAWYQHKPGQAPSLLI |
| C1978-A10- | | SGASSRATGVPDRFSGSGSGTDFTLAISRLEPEDSAVYYCQHYDSSPSW |
| aa | | TFGQGTKVEIK |
| VL | | |
| BCMA_EBB- | 229 | MALPVTALLLPLALLLHAARPEVQLVETGGGLVQPGGSLRLSCAASGF |
| C1978-A10- | | TFSSYAMSWVRQAPGKGLEWVSAISGSGGSTYYADSVKGRFTMSREN |
| aa | | DKNSVFLQMNSLRVEDTGVYYCARANYKRELRYYYGMDVWGQGTM |
| Full CART | | VTVSSGGGGSGGGGSGGGGSEIVMTQSPGTLSLSPGESATLSCRASQR |
| | | VASNYLAWYQHKPGQAPSLLISGASSRATGVPDRFSGSGSGTDFTLAIS |
| | | RLEPEDSAVYYCQHYDSSPSWTFGQGTKVEIKTTTPAPRPPTPAPTIAS |
| | | QPLSLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELRVK |
| | | FSRSADAPAYKQGQNQLYNELNLGRREEYDVLDKRRGRDPEMGGKP |
| | | RRKNPQEGLYNELQKDKMAEAYSEIGMKGERRRGKGHDGLYQGLST |
| | | ATKDTYDALHMQALPPR |
| BCMA_EBB- | 250 | ATGGCCCTCCCTGTCACCGCCCTGCTGCTTCCGCTGGCTCTTCTGCT |
| C1978-A10- | | CCACGCCGCTCGGCCCGAAGTGCAACTGGTGGAAACCGGTGGAGG |
| nt | | ACTCGTGCAGCCTGGCGGCAGCCTCCGGCTGAGCTGCGCCGCTTCG |
| Full CART | | GGATTCACCTTTTCCTCCTACGCGATGTCTTGGGTCAGACAGGCCCC |
| | | CGGAAAGGGGCTGGAATGGGTGTCAGCCATCTCCGGCTCCGGCGGA |
| | | TCAACGTACTACGCCGACTCCGTGAAAGGCCGGTTCACCATGTCGC |
| | | GCGAGAATGACAAGAACTCCGTGTTCCTGCAAATGAACTCCCTGAG |
| | | GGTGGAGGACACCGGAGTGTACTATTGTGCGCGCGCCAACTACAAG |
| | | AGAGAGCTGCGGTACTACTACGGAATGGACGTCTGGGGACAGGGA |
| | | ACTATGGTGACCGTGTCATCCGGTGGAGGGGGAAGCGGCGGTGGA |
| | | GGCAGCGGGGGCGGGGGTTCAGAAATTGTCATGACCCAGTCCCCGG |
| | | GAACTCTTTCCCTCTCCCCCGGGGAATCCGCGACTTTGTCCTGCCGG |
| | | GCCAGCCAGCGCGTGGCCTCGAACTACCTCGCATGGTACCAGCATA |
| | | AGCCAGGCCAAGCCCCTTCCCTGCTGATTTCCGGGGCTAGCAGCCG |
| | | CGCCACTGGCGTGCCGGATAGGTTCTCGGGAAGCGGCTCGGGTACC |
| | | GATTTCACCCTGGCAATCTCGCGGCTGGAACCGGAGGATTCGGCCG |
| | | TGTACTACTGCCAGCACTATGACTCATCCCCCTCCTGGACATTCGGA |
| | | CAGGGCACCAAGGTCGAGATCAAGACCACTACCCCAGCACCGAGG |
| | | CCACCCACCCCGGCTCCTACCATCGCCTCCCAGCCTCTGTCCCTGCG |
| | | TCCGGAGGCATGTAGACCCGCAGCTGGTGGGGCCGTGCATACCCGG |
| | | GGTCTTGACTTCGCCTGCGATATCTACATTTGGGCCCCTCTGGCTGG |
| | | TACTTGCGGGGTCCTGCTGCTTTCACTCGTGATCACTCTTTACTGTA |
| | | AGCGCGGTCGGAAGAAGCTGCTGTACATCTTTAAGCAACCCTTCAT |
| | | GAGGCCTGTGCAGACTACTCAAGAGGAGGACGGCTGTTCATGCCGG |
| | | TTCCCAGAGGAGGAGGAAGGCGGCTGCGAACTGCGCGTGAAATTC |
| | | AGCCGCAGCGCAGATGCTCCAGCCTACAAGCAGGGGCAGAACCAG |
| | | CTCTACAACGAACTCAATCTTGGTCGGAGAGAGGAGTACGACGTGC |
| | | TGGACAAGCGGAGAGGACGGGACCCAGAAATGGGCGGGAAGCCGC |
| | | GCAGAAAGAATCCCCAAGAGGGCCTGTACAACGAGCTCCAAAAGG |
| | | ATAAGATGGCAGAAGCCTATAGCGAGATTGGTATGAAAGGGGAAC |
| | | GCAGAAGAGGCAAAGGCCACGACGGACTGTACCAGGGACTCAGCA |
| | | CCGCCACCAAGGACACCTATGACGCTCTTCACATGCAGGCCCTGCC |
| | | GCCTCGG |

BCMA_EBB-C1978-D4

| | | |
|---|---|---|
| BCMA_EBB- | 146 | EVQLLETGGGLVQPGGSLRLSCAASGFSFSSYAMSWVRQAPGKGLEW |
| C1978-D4-aa | | VSAISGSGGSTYYADSVKGRFTISRDNSKNTLYLQMNSLRAEDTAVYY |
| ScFv domain | | CAKALVGATGAFDIWGQGTLVTVSSGGGGSGGGGSGGGGSEIVLTQS |
| | | PGTLSLSPGERATLSCRASQSLSSNFLAWYQQKPGQAPGLLIYGASNW |
| | | ATGTPDRFSGSGSGTDFTLTITRLEPEDFAVYYCQYYGTSPMYTFGQGT |
| | | KVEIK |
| BCMA_EBB- | 167 | GAAGTGCAGCTGCTCGAAACCGGTGGAGGGCTGGTGCAGCCAGGG |
| C1978-D4-nt | | GGCTCCCTGAGGCTTTCATGCGCCGCTAGCGGATTCTCCTTCTCCTC |
| ScFv domain | | TTACGCCATGTCGTGGGTCCGCCAAGCCCCTGGAAAAGGCCTGGAA |
| | | TGGGTGTCCGCGATTTCCGGGAGCGGAGGTTCGACCTATTACGCCG |
| | | ACTCCGTGAAGGGCCGCTTTACCATCTCCCGGGATAACTCCAAGAA |
| | | CACTCTGTACCTCCAAATGAACTCGCTGAGAGCCGAGGACACCGCC |
| | | GTGTATTACTGCGCGAAGGCGCTGGTCGGCGCGACTGGGGCATTCG |
| | | ACATCTGGGGACAGGGAACTCTTGTGACCGTGTCGAGCGGAGGCGG |
| | | CGGCTCCGGCGGAGGAGGGAGCGGGGGCGGTGGTTCCGAAATCGT |
| | | GTTGACTCAGTCCCCGGGAACCCTGAGCTTGTCACCCGGGGAGCGG |
| | | GCCACTCTCTCCTGTCGCGCCTCCCAATCGCTCTCATCCAATTTCCT |
| | | GGCCTGGTACCAGCAGAAGCCCGGACAGGCCCCGGGCCTGCTCATC |
| | | TACGGCGCTTCAAACTGGGCAACGGGAACCCCTGATCGGTTCAGCG |
| | | GAAGCGGATCGGGTACTGACTTTACCCTGACCATCACCAGACTGGA |
| | | ACCGGAGGACTTCGCCGTGTACTACTGCCAGTACTACGGCACCTCC |
| | | CCCATGTACACATTCGGACAGGGTACCAAGGTCGAGATTAAG |
| BCMA_EBB- | 188 | EVQLLETGGGLVQPGGSLRLSCAASGFSFSSYAMSWVRQAPGKGLEW |
| C1978-D4-aa | | VSAISGSGGSTYYADSVKGRFTISRDNSKNTLYLQMNSLRAEDTAVYY |
| VH | | CAKALVGATGAFDIWGQGTLVTVSS |
| BCMA_EBB- | 209 | EIVLTQSPGTLSLSPGERATLSCRASQSLSSNFLAWYQQKPGQAPGLLIY |
| C1978-D4-aa | | GASNWATGTPDRFSGSGSGTDFTLTITRLEPEDFAVYYCQYYGTSPMY |
| VL | | TFGQGTKVEIK |
| BCMA_EBB- | 230 | MALPVTALLLPLALLLHAARPEVQLLETGGGLVQPGGSLRLSCAASGF |
| C1978-D4-aa | | SFSSYAMSWVRQAPGKGLEWVSAISGSGGSTYYADSVKGRFTISRDNS |
| Full CART | | KNTLYLQMNSLRAEDTAVYYCAKALVGATGAFDIWGQGTLVTVSSG |
| | | GGGSGGGGSGGGGSEIVLTQSPGTLSLSPGERATLSCRASQSLSSNFLA |
| | | WYQQKPGQAPGLLIYGASNWATGTPDRFSGSGSGTDFTLTITRLEPEDF |
| | | AVYYCQYYGTSPMYTFGQGTKVEIKTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYD |
| | | ALHMQALPPR |
| BCMA_EBB- | 251 | ATGGCCCTCCCTGTCACCGCCCTGCTGCTTCCGCTGGCTCTTCTGCT |
| C1978-D4-nt | | CCACGCCGCTCGGCCCGAAGTGCAGCTGCTCGAAACCGGTGGAGGG |
| Full CART | | CTGGTGCAGCCAGGGGGCTCCCTGAGGCTTTCATGCGCCGCTAGCG |
| | | GATTCTCCTTCTCCTCTTACGCCATGTCGTGGGTCCGCCAAGCCCCT |
| | | GGAAAAGGCCTGGAATGGGTGTCCGCGATTTCCGGGAGCGGAGGTT |
| | | CGACCTATTACGCCGACTCCGTGAAGGGCCGCTTTACCATCTCCCG |
| | | GGATAACTCCAAGAACACTCTGTACCTCCAAATGAACTCGCTGAGA |
| | | GCCGAGGACACCGCCGTGTATTACTGCGCGAAGGCGCTGGTCGGCG |
| | | CGACTGGGGCATTCGACATCTGGGGACAGGGAACTCTTGTGACCGT |
| | | GTCGAGCGGAGGCGGCGGCTCCGGCGGAGGAGGGAGCGGGGGCGG |
| | | TGGTTCCGAAATCGTGTTGACTCAGTCCCCGGGAACCCTGAGCTTGT |
| | | CACCCGGGGAGCGGGCCACTCTCTCCTGTCGCGCCTCCCAATCGCT |
| | | CTCATCCAATTTCCTGGCCTGGTACCAGCAGAAGCCCGGACAGGCC |
| | | CCGGGCCTGCTCATCTACGGCGCTTCAAACTGGGCAACGGGAACCC |
| | | CTGATCGGTTCAGCGGAAGCGGATCGGGTACTGACTTTACCCTGAC |
| | | CATCACCAGACTGGAACCGGAGGACTTCGCCGTGTACTACTGCCAG |
| | | TACTACGGCACCTCCCCCATGTACACATTCGGACAGGGTACCAAGG |
| | | TCGAGATTAAGACCACTACCCCAGCACCGAGGCCACCCACCCCGGC |
| | | TCCTACCATCGCCTCCCAGCCTCTGTCCCTGCGTCCGGAGGCATGTA |
| | | GACCCGCAGCTGGTGGGGCCGTGCATACCCGGGGTCTTGACTTCGC |
| | | CTGCGATATCTACATTTGGGCCCCTCTGGCTGGTACTTGCGGGGTCC |
| | | TGCTGCTTTCACTCGTGATCACTCTTTACTGTAAGCGCGGTCGGAAG |
| | | AAGCTGCTGTACATCTTTAAGCAACCCTTCATGAGGCCTGTGCAGA |
| | | CTACTCAAGAGGAGGACGGCTGTTCATGCCGGTTCCCAGAGGAGGA |
| | | GGAAGGCGGCTGCGAACTGCGCGTGAAATTCAGCCGCAGCGCAGA |
| | | TGCTCCAGCCTACAAGCAGGGGCAGAACCAGCTCTACAACGAACTC |
| | | AATCTTGGTCGGAGAGAGGAGTACGACGTGCTGGACAAGCGGAGA |
| | | GGACGGGACCCAGAAATGGGCGGGAAGCCGCGCAGAAAGAATCCC |
| | | CAAGAGGGCCTGTACAACGAGCTCCAAAAGGATAAGATGGCAGAA |
| | | GCCTATAGCGAGATTGGTATGAAAGGGGAACGCAGAAGAGGCAAA |
| | | GGCCACGACGGACTGTACCAGGGACTCAGCACCGCCACCAAGGAC |
| | | ACCTATGACGCTCTTCACATGCAGGCCCTGCCGCCTCGG |

BCMA_EBB-C1980-A2

| | | |
|---|---|---|
| BCMA_EBB- | 147 | EVQLLESGGGLVQPGGSLRLSCAASGFTFSSYAMSWVRQAPGKGLEW |
| C1980-A2-aa | | VSAISGSGGSTYYADSVKGRFTISRDNSKNTLYLQMNSLRAEDTAVYY |
| ScFv domain | | CVLWFGEGFDPWGQGTLVTVSSGGGGSGGGGSGGGGSDIVLTQSPLS |
| | | LPVTPGEPASISCRSSQSLLHSNGYNYLDWYLQKPGQSPQLLIYLGSNR |
| | | ASGVPDRFSGSGSGTDFTLKISRVEAEDVGVYYCMQALQTPLTFGGGT |
| | | KVDIK |
| BCMA_EBB- | 168 | GAAGTGCAGCTGCTTGAGAGCGGTGGAGGTCTGGTGCAGCCCGGG |
| C1980-A2-nt | | GGATCACTGCGCCTGTCCTGTGCCGCGTCCGGTTTCACTTTCTCCTC |
| ScFv domain | | GTACGCCATGTCGTGGGTCAGACAGGCACCGGGAAAGGGACTGGA |
| | | ATGGGTGTCAGCCATTTCGGGTTCGGGGGGCAGCACCTACTACGCT |
| | | GACTCCGTGAAGGGCCGGTTCACCATTTCCCGCGACAACTCCAAGA |
| | | ACACCTTGTACCTCCAAATGAACTCCCTGCGGGCCGAAGATACCGC |
| | | CGTGTATTACTGCGTGCTGTGGTTCGGAGAGGGATTCGACCCGTGG |
| | | GGACAAGGAACACTCGTGACTGTGTCATCCGGCGGAGGCGGCAGC |
| | | GGTGGCGGCGGTTCCGGCGGCGGCGGATCTGACATCGTGTTGACCC |
| | | AGTCCCCTCTGAGCCTGCCGGTCACTCCTGGCGAACCAGCCAGCAT |
| | | CTCCTGCCGGTCGAGCCAGTCCCTCCTGCACTCCAATGGGTACAACT |
| | | ACCTCGATTGGTATCTGCAAAAGCCGGGCCAGAGCCCCCAGCTGCT |
| | | GATCTACCTTGGGTCAAACCGCGCTTCCGGGGTGCCTGATAGATTCT |
| | | CCGGGTCCGGGAGCGGAACCGACTTTACCCTGAAAATCTCGAGGGT |
| | | GGAGGCCGAGGACGTCGGAGTGTACTACTGCATGCAGGCGCTCCAG |
| | | ACTCCCCTGACCTTCGGAGGAGGAACGAAGGTCGACATCAAGA |
| BCMA_EBB- | 189 | EVQLLESGGGLVQPGGSLRLSCAASGFTFSSYAMSWVRQAPGKGLEW |
| C1980-A2-aa | | VSAISGSGGSTYYADSVKGRFTISRDNSKNTLYLQMNSLRAEDTAVYY |
| VH | | CVLWFGEGFDPWGQGTLVTVSS |
| BCMA_EBB- | 210 | DIVLTQSPLSLPVTPGEPASISCRSSQSLLHSNGYNYLDWYLQKPGQSP |
| C1980-A2-aa | | QLLIYLGSNRASGVPDRFSGSGSGTDFTLKISRVEAEDVGVYYCMQAL |
| VL | | QTPLTFGGGTKVDIK |
| BCMA_EBB- | 231 | MALPVTALLLPLALLLHAARPEVQLLESGGGLVQPGGSLRLSCAASGF |
| C1980-A2-aa | | TFSSYAMSWVRQAPGKGLEWVSAISGSGGSTYYADSVKGRFTISRDNS |
| Full CART | | KNTLYLQMNSLRAEDTAVYYCVLWFGEGFDPWGQGTLVTVSSGGGG |
| | | SGGGGSGGGGSDIVLTQSPLSLPVTPGEPASISCRSSQSLLHSNGYNYLD |
| | | WYLQKPGQSPQLLIYLGSNRASGVPDRFSGSGSGTDFTLKISRVEAEDV |
| | | GVYYCMQALQTPLTFGGGTKVDIKTTTPAPRPPTPAPTIASQPLSLRPE |
| | | ACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAP |
| | | AYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDA |
| | | LHMQALPPR |
| BCMA_EBB- | 252 | ATGGCCCTCCCTGTCACCGCCCTGCTGCTTCCGCTGGCTCTTCTGCT |
| C1980-A2-nt | | CCACGCCGCTCGGCCCGAAGTGCAGCTGCTTGAGAGCGGTGGAGGT |
| Full CART | | CTGGTGCAGCCCGGGGGATCACTGCGCCTGTCCTGTGCCGCGTCCG |
| | | GTTTCACTTTCTCCTCGTACGCCATGTCGTGGGTCAGACAGGCACCG |
| | | GGAAAGGGACTGGAATGGGTGTCAGCCATTTCGGGTTCGGGGGGC |
| | | AGCACCTACTACGCTGACTCCGTGAAGGGCCGGTTCACCATTTCCC |
| | | GCGACAACTCCAAGAACACCTTGTACCTCCAAATGAACTCCCTGCG |
| | | GGCCGAAGATACCGCCGTGTATTACTGCGTGCTGTGGTTCGGAGAG |
| | | GGATTCGACCCGTGGGGACAAGGAACACTCGTGACTGTGTCATCCG |
| | | GCGGAGGCGGCAGCGGTGGCGGCGGTTCCGGCGGCGGCGGATCTG |
| | | ACATCGTGTTGACCCAGTCCCCTCTGAGCCTGCCGGTCACTCCTGGC |
| | | GAACCAGCCAGCATCTCCTGCCGGTCGAGCCAGTCCCTCCTGCACT |
| | | CCAATGGGTACAACTACCTCGATTGGTATCTGCAAAAGCCGGGCCA |
| | | GAGCCCCCAGCTGCTGATCTACCTTGGGTCAAACCGCGCTTCCGGG |
| | | GTGCCTGATAGATTCTCCGGGTCCGGGAGCGGAACCGACTTTACCC |
| | | TGAAAATCTCGAGGGTGGAGGCCGAGGACGTCGGAGTGTACTACTG |
| | | CATGCAGGCGCTCCAGACTCCCCTGACCTTCGGAGGAGGAACGAAG |
| | | GTCGACATCAAGACCACTACCCCAGCACCGAGGCCACCCACCCCGG |
| | | CTCCTACCATCGCCTCCCAGCCTCTGTCCCTGCGTCCGGAGGCATGT |
| | | AGACCCGCAGCTGGTGGGGCCGTGCATACCCGGGGTCTTGACTTCG |
| | | CCTGCGATATCTACATTTGGGCCCCTCTGGCTGGTACTTGCGGGGTC |
| | | CTGCTGCTTTCACTCGTGATCACTCTTTACTGTAAGCGCGGTCGGAA |
| | | GAAGCTGCTGTACATCTTTAAGCAACCCTTCATGAGGCCTGTGCAG |
| | | ACTACTCAAGAGGAGGACGGCTGTTCATGCCGGTTCCCAGAGGAGG |
| | | AGGAAGGCGGCTGCGAACTGCGCGTGAAATTCAGCCGCAGCGCAG |
| | | ATGCTCCAGCCTACAAGCAGGGGCAGAACCAGCTCTACAACGAACT |
| | | CAATCTTGGTCGGAGAGAGGAGTACGACGTGCTGGACAAGCGGAG |
| | | AGGACGGGACCCAGAAATGGGCGGGAAGCCGCGCAGAAAGAATCC |
| | | CCAAGAGGGCCTGTACAACGAGCTCCAAAAGGATAAGATGGCAGA |
| | | AGCCTATAGCGAGATTGGTATGAAAGGGGAACGCAGAAGAGGCAA |
| | | AGGCCACGACGGACTGTACCAGGGACTCAGCACCGCCACCAAGGA |
| | | CACCTATGACGCTCTTCACATGCAGGCCCTGCCGCCTCGG |

BCMA_EBB-C1981-C3

| | | |
|---|---|---|
| BCMA_EBB- | 148 | QVQLVESGGGLVQPGGSLRLSCAASGFTFSSYAMSWVRQAPGKGLEW |
| C1981-C3-aa | | VSAISGSGGSTYYADSVKGRFTISRDNSKNTLYLQMNSLRAEDTAVYY |
| ScFv domain | | CAKVGYDSSGYYRDYYGMDVWGQGTTVTVSSGGGGSGGGGSGGGG |
| | | SEIVLTQSPGTLSLSPGERATLSCRASQSVSSSYLAWYQQKPGQAPRLLI |
| | | YGTSSRATGISDRFSGSGSGTDFTLTISRLEPEDFAVYYCQHYGNSPPKF |
| | | TFGPGTKLEIK |
| BCMA_EBB- | 169 | CAAGTGCAGCTCGTGGAGTCAGGCGGAGGACTGGTGCAGCCCGGG |
| C1981-C3-nt | | GGCTCCCTGAGACTTTCCTGCGCGGCATCGGGTTTTACCTTCTCCTC |
| ScFv domain | | CTATGCTATGTCCTGGGTGCGCCAGGCCCCGGGAAAGGGACTGGAA |
| | | TGGGTGTCCGCAATCAGCGGTAGCGGGGGCTCAACATACTACGCCG |
| | | ACTCCGTCAAGGGTCGCTTCACTATTTCCCGGGACAACTCCAAGAA |
| | | TACCCTGTACCTCCAAATGAACAGCCTCAGGGCCGAGGATACTGCC |
| | | GTGTACTACTGCGCCAAAGTCGGATACGATAGCTCCGGTTACTACC |
| | | GGGACTACTACGGAATGGACGTGTGGGGACAGGGCACCACCGTGA |
| | | CCGTGTCAAGCGGCGGAGGCGGTTCAGGAGGGGGAGGCTCCGGCG |
| | | GTGGAGGGTCCGAAATCGTCCTGACTCAGTCGCCTGGCACTCTGTC |
| | | GTTGTCCCCGGGGGAGCGCGCTACCCTGTCGTGTCGGGCGTCGCAG |
| | | TCCGTGTCGAGCTCCTACCTCGCGTGGTACCAGCAGAAGCCCGGAC |
| | | AGGCCCCTAGACTTCTGATCTACGGCACTTCTTCACGCGCCACCGG |
| | | GATCAGCGACAGGTTCAGCGGCTCCGGCTCCGGGACCGACTTCACC |
| | | CTGACCATTAGCCGGCTGGAGCCTGAAGATTTCGCCGTGTATTACT |
| | | GCCAACACTACGGAAACTCGCCGCCAAAGTTCACGTTCGGACCCGG |
| | | AACCAAGCTGGAAATCAAG |
| BCMA_EBB- | 190 | QVQLVESGGGLVQPGGSLRLSCAASGFTFSSYAMSWVRQAPGKGLEW |
| C1981-C3-aa | | VSAISGSGGSTYYADSVKGRFTISRDNSKNTLYLQMNSLRAEDTAVYY |
| VH | | CAKVGYDSSGYYRDYYGMDVWGQGTTVTVSS |
| BCMA_EBB- | 211 | EIVLTQSPGTLSLSPGERATLSCRASQSVSSSYLAWYQQKPGQAPRLLI |
| C1981-C3-aa | | YGTSSRATGISDRFSGSGSGTDFTLTISRLEPEDFAVYYCQHYGNSPPKF |
| VL | | TFGPGTKLEIK |
| BCMA_EBB- | 232 | MALPVTALLLPLALLLHAARPQVQLVESGGGLVQPGGSLRLSCAASGF |
| C1981-C3-aa | | TFSSYAMSWVRQAPGKGLEWVSAISGSGGSTYYADSVKGRFTISRDNS |
| Full CART | | KNTLYLQMNSLRAEDTAVYYCAKVGYDSSGYYRDYYGMDVWGQGT |
| | | TVTVSSGGGGSGGGGSGGGGSEIVLTQSPGTLSLSPGERATLSCRASQS |
| | | VSSSYLAWYQQKPGQAPRLLIYGTSSRATGISDRFSGSGSGTDFTLTISR |
| | | LEPEDFAVYYCQHYGNSPPKFTFGPGTKLEIKTTTPAPRPPTPAPTIASQ |
| | | PLSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKF |
| | | SRSADAPAYKQGQNQLYNELNLGRREEYDVLDKRRGRDPEMGGKPR |
| | | RKNPQEGLYNELQKDKMAEAYSEIGMKGERRRGKGHDGLYQGLSTA |
| | | TKDTYDALHMQALPPR |
| BCMA_EBB- | 253 | ATGGCCCTCCCTGTCACCGCCCTGCTGCTTCCGCTGGCTCTTCTGCT |
| C1981-C3-nt | | CCACGCCGCTCGGCCCCAAGTGCAGCTCGTGGAGTCAGGCGGAGGA |
| Full CART | | CTGGTGCAGCCCGGGGGCTCCCTGAGACTTTCCTGCGCGGCATCGG |
| | | GTTTTACCTTCTCCTCCTATGCTATGTCCTGGGTGCGCCAGGCCCCG |
| | | GGAAAGGGACTGGAATGGGTGTCCGCAATCAGCGGTAGCGGGGGC |
| | | TCAACATACTACGCCGACTCCGTCAAGGGTCGCTTCACTATTTCCCG |
| | | GGACAACTCCAAGAATACCCTGTACCTCCAAATGAACAGCCTCAGG |
| | | GCCGAGGATACTGCCGTGTACTACTGCGCCAAAGTCGGATACGATA |
| | | GCTCCGGTTACTACCGGGACTACTACGGAATGGACGTGTGGGGACA |
| | | GGGCACCACCGTGACCGTGTCAAGCGGCGGAGGCGGTTCAGGAGG |
| | | GGGAGGCTCCGGCGGTGGAGGGTCCGAAATCGTCCTGACTCAGTCG |
| | | CCTGGCACTCTGTCGTTGTCCCCGGGGGAGCGCGCTACCCTGTCGTG |
| | | TCGGGCGTCGCAGTCCGTGTCGAGCTCCTACCTCGCGTGGTACCAG |
| | | CAGAAGCCCGGACAGGCCCCTAGACTTCTGATCTACGGCACTTCTT |
| | | CACGCGCCACCGGGATCAGCGACAGGTTCAGCGGCTCCGGCTCCGG |
| | | GACCGACTTCACCCTGACCATTAGCCGGCTGGAGCCTGAAGATTTC |
| | | GCCGTGTATTACTGCCAACACTACGGAAACTCGCCGCCAAAGTTCA |
| | | CGTTCGGACCCGGAACCAAGCTGGAAATCAAGACCACTACCCCAGC |
| | | ACCGAGGCCACCCACCCCGGCTCCTACCATCGCCTCCCAGCCTCTGT |
| | | CCCTGCGTCCGGAGGCATGTAGACCCGCAGCTGGTGGGGCCGTGCA |
| | | TACCCGGGGTCTTGACTTCGCCTGCGATATCTACATTTGGGCCCCTC |
| | | TGGCTGGTACTTGCGGGGTCCTGCTGCTTTCACTCGTGATCACTCTT |
| | | TACTGTAAGCGCGGTCGGAAGAAGCTGCTGTACATCTTTAAGCAAC |
| | | CCTTCATGAGGCCTGTGCAGACTACTCAAGAGGAGGACGGCTGTTC |
| | | ATGCCGGTTCCCAGAGGAGGAGGAAGGCGGCTGCGAACTGCGCGT |
| | | GAAATTCAGCCGCAGCGCAGATGCTCCAGCCTACAAGCAGGGGCA |
| | | GAACCAGCTCTACAACGAACTCAATCTTGGTCGGAGAGAGGAGTAC |
| | | GACGTGCTGGACAAGCGGAGAGGACGGGACCCAGAAATGGGCGGG |
| | | AAGCCGCGCAGAAAGAATCCCCAAGAGGGCCTGTACAACGAGCTC |
| | | CAAAAGGATAAGATGGCAGAAGCCTATAGCGAGATTGGTATGAAA |
| | | GGGGAACGCAGAAGAGGCAAAGGCCACGACGGACTGTACCAGGGA |
| | | CTCAGCACCGCCACCAAGGACACCTATGACGCTCTTCACATGCAGG |
| | | CCCTGCCGCCTCGG |

BCMA_EBB-C1978-G4

| | | |
|---|---|---|
| BCMA_EBB- | 149 | EVQLVESGGGLVQPGGSLRLSCAASGFTFSSYAMSWVRQAPGKGLEW |
| C1978-G4-aa | | VSAISGSGGSTYYADSVKGRFTISRDNSKNTLYLQMNSLRAEDTAVYY |
| ScFv domain | | CAKMGWSSGYLGAFDIWGQGTTVTVSSGGGGSGGGGSGGGGSEIVLT |
| | | QSPGTLSLSPGERATLSCRASQSVASSFLAWYQQKPGQAPRLLIYGASG |
| | | RATGIPDRFSGSGSGTDFTLTISRLEPEDFAVYYCQHYGGSPRLTFGGG |
| | | TKVDIK |
| BCMA_EBB- | 170 | GAAGTCCAACTGGTGGAGTCCGGGGGAGGGCTCGTGCAGCCCGGA |
| C1978-G4-nt | | GGCAGCCTTCGGCTGTCGTGCGCCGCCTCCGGGTTCACGTTCTCATC |
| ScFv domain | | CTACGCGATGTCGTGGGTCAGACAGGCACCAGGAAAGGGACTGGA |
| | | ATGGGTGTCCGCCATTAGCGGCTCCGGCGGTAGCACCTACTATGCC |
| | | GACTCAGTGAAGGGAAGGTTCACTATCTCCCGCGACAACAGCAAGA |
| | | ACACCCTGTACCTCCAAATGAACTCTCTGCGGGCCGAGGATACCGC |
| | | GGTGTACTATTGCGCCAAGATGGGTTGGTCCAGCGGATACTTGGGA |
| | | GCCTTCGACATTTGGGGACAGGGCACTACTGTGACCGTGTCCTCCG |
| | | GGGGTGGCGGATCGGGAGGCGGCGGCTCGGGTGGAGGGGGTTCCG |
| | | AAATCGTGTTGACCCAGTCACCGGGAACCCTCTCGCTGTCCCCGGG |
| | | AGAACGGGCTACACTGTCATGTAGAGCGTCCCAGTCCGTGGCTTCC |
| | | TCGTTCCTGGCCTGGTACCAGCAGAAGCCGGGACAGGCACCCCGCC |
| | | TGCTCATCTACGGAGCCAGCGGCCGGGCGACCGGCATCCCTGACCG |
| | | CTTCTCCGGTTCCGGCTCGGGCACCGACTTTACTCTGACCATTAGCA |
| | | GGCTTGAGCCCGAGGATTTTGCCGTGTACTACTGCCAACACTACGG |
| | | GGGGAGCCCTCGCCTGACCTTCGGAGGCGGAACTAAGGTCGATATC |
| | | AAAA |
| BCMA_EBB- | 191 | EVQLVESGGGLVQPGGSLRLSCAASGFTFSSYAMSWVRQAPGKGLEW |
| C1978-G4-aa | | VSAISGSGGSTYYADSVKGRFTISRDNSKNTLYLQMNSLRAEDTAVYY |
| VH | | CAKMGWSSGYLGAFDIWGQGTTVTVSS |
| BCMA_EBB- | 212 | EIVLTQSPGTLSLSPGERATLSCRASQSVASSFLAWYQQKPGQAPRLLI |
| C1978-G4-aa | | YGASGRATGIPDRFSGSGSGTDFTLTISRLEPEDFAVYYCQHYGGSPRL |
| VL | | TFGGGTKVDIK |
| BCMA_EBB- | 233 | MALPVTALLLPLALLLHAARPEVQLVESGGGLVQPGGSLRLSCAASGF |
| C1978-G4-aa | | TFSSYAMSWVRQAPGKGLEWVSAISGSGGSTYYADSVKGRFTISRDNS |
| Full CART | | KNTLYLQMNSLRAEDTAVYYCAKMGWSSGYLGAFDIWGQGTTVTVS |
| | | SGGGGSGGGGSGGGGSEIVLTQSPGTLSLSPGERATLSCRASQSVASSF |
| | | LAWYQQKPGQAPRLLIYGASGRATGIPDRFSGSGSGTDFTLTISRLEPE |
| | | DFAVYYCQHYGGSPRLTFGGGTKVDIKTTTPAPRPPTPAPTIASQPLSL |
| | | RPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDT |
| | | YDALHMQALPPR |
| BCMA_EBB- | 254 | ATGGCCCTCCCTGTCACCGCCCTGCTGCTTCCGCTGGCTCTTCTGCT |
| C1978-G4-nt | | CCACGCCGCTCGGCCCGAAGTCCAACTGGTGGAGTCCGGGGGAGG |
| Full CART | | GCTCGTGCAGCCCGGAGGCAGCCTTCGGCTGTCGTGCGCCGCCTCC |
| | | GGGTTCACGTTCTCATCCTACGCGATGTCGTGGGTCAGACAGGCAC |
| | | CAGGAAAGGGACTGGAATGGGTGTCCGCCATTAGCGGCTCCGGCG |
| | | GTAGCACCTACTATGCCGACTCAGTGAAGGGAAGGTTCACTATCTC |
| | | CCGCGACAACAGCAAGAACACCCTGTACCTCCAAATGAACTCTCTG |
| | | CGGGCCGAGGATACCGCGGTGTACTATTGCGCCAAGATGGGTTGGT |
| | | CCAGCGGATACTTGGGAGCCTTCGACATTTGGGGACAGGGCACTAC |
| | | TGTGACCGTGTCCTCCGGGGGTGGCGGATCGGGAGGCGGCGGCTCG |
| | | GGTGGAGGGGGTTCCGAAATCGTGTTGACCCAGTCACCGGGAACCC |
| | | TCTCGCTGTCCCCGGGAGAACGGGCTACACTGTCATGTAGAGCGTC |
| | | CCAGTCCGTGGCTTCCTCGTTCCTGGCCTGGTACCAGCAGAAGCCG |
| | | GGACAGGCACCCCGCCTGCTCATCTACGGAGCCAGCGGCCGGGCGA |
| | | CCGGCATCCCTGACCGCTTCTCCGGTTCCGGCTCGGGCACCGACTTT |
| | | ACTCTGACCATTAGCAGGCTTGAGCCCGAGGATTTTGCCGTGTACT |
| | | ACTGCCAACACTACGGGGGGAGCCCTCGCCTGACCTTCGGAGGCGG |
| | | AACTAAGGTCGATATCAAAACCACTACCCCAGCACCGAGGCCACCC |
| | | ACCCCGGCTCCTACCATCGCCTCCCAGCCTCTGTCCCTGCGTCCGGA |
| | | GGCATGTAGACCCGCAGCTGGTGGGGCCGTGCATACCCGGGGTCTT |
| | | GACTTCGCCTGCGATATCTACATTTGGGCCCCTCTGGCTGGTACTTG |
| | | CGGGGTCCTGCTGCTTTCACTCGTGATCACTCTTTACTGTAAGCGCG |
| | | GTCGGAAGAAGCTGCTGTACATCTTTAAGCAACCCTTCATGAGGCC |
| | | TGTGCAGACTACTCAAGAGGAGGACGGCTGTTCATGCCGGTTCCCA |
| | | GAGGAGGAGGAAGGCGGCTGCGAACTGCGCGTGAAATTCAGCCGC |
| | | AGCGCAGATGCTCCAGCCTACAAGCAGGGGCAGAACCAGCTCTAC |
| | | AACGAACTCAATCTTGGTCGGAGAGAGGAGTACGACGTGCTGGAC |
| | | AAGCGGAGAGGACGGGACCCAGAAATGGGCGGGAAGCCGCGCAG |
| | | AAAGAATCCCCAAGAGGGCCTGTACAACGAGCTCCAAAAGGATAA |
| | | GATGGCAGAAGCCTATAGCGAGATTGGTATGAAAGGGGAACGCAG |
| | | AAGAGGCAAAGGCCACGACGGACTGTACCAGGGACTCAGCACCGC |
| | | CACCAAGGACACCTATGACGCTCTTCACATGCAGGCCCTGCCGCCT |
| | | CGG |

TABLE 7
Exemplary Heavy Chain Variable Domain CDRs according to the Kabat Numbering
Scheme (Kabat et al. (1991), “Sequences of Proteins of Immunological
Interest,” 5th Ed. Public Health Service, National Institutes of Health,
Bethesda, MD)

| | | SEQ | | SEQ | | SEQ |
|---|---|---|---|---|---|---|
| | | ID | | ID | | ID |
| Candidate | HCDR1 | NO | HCDR2 | NO | HCDR3 | NO |
| 139109 | NHGMS | 1118 | GIVYSGSTYYAAS | 1158 | HGGESDV | 1198 |
| | | | VKG | | | |
| 139103 | NYAMS | 1119 | GISRSGENTYYAD | 1159 | SPAHYYGGMD | 1199 |
| | | | SVKG | | V | |
| 139105 | DYAMH | 1120 | GISWNSGSIGYAD | 1160 | HSFLAY | 1200 |
| | | | SVKG | | | |
| 139111 | NHGMS | 1121 | GIVYSGSTYYAAS | 1161 | HGGESDV | 1201 |
| | | | VKG | | | |
| 139100 | NFGIN | 1122 | WINPKNNNTNYA | 1162 | GPYYYQSYMD | 1202 |
| | | | QKFQG | | V | |
| 139101 | SDAMT | 1123 | VISGSGGTTYYAD | 1163 | LDSSGYYYARG | 1203 |
| | | | SVKG | | PRY | |
| 139102 | NYGIT | 1124 | WISAYNGNTNYA | 1164 | GPYYYYMDV | 1204 |
| | | | QKFQG | | | |
| 139104 | NHGMS | 1125 | GIVYSGSTYYAAS | 1165 | HGGESDV | 1205 |
| | | | VKG | | | |
| 139106 | NHGMS | 1126 | GIVYSGSTYYAAS | 1166 | HGGESDV | 1206 |
| | | | VKG | | | |
| 139107 | NHGMS | 1127 | GIVYSGSTYYAAS | 1167 | HGGESDV | 1207 |
| | | | VKG | | | |
| 139108 | DYYMS | 1128 | YISSSGSTIYYADS | 1168 | ESGDGMDV | 1208 |
| | | | VKG | | | |
| 139110 | DYYMS | 1129 | YISSSGNTIYYADS | 1169 | STMVREDY | 1209 |
| | | | VKG | | | |
| 139112 | NHGMS | 1130 | GIVYSGSTYYAAS | 1170 | HGGESDV | 1210 |
| | | | VKG | | | |
| 139113 | NHGMS | 1131 | GIVYSGSTYYAAS | 1171 | HGGESDV | 1211 |
| | | | VKG | | | |
| 139114 | NHGMS | 1132 | GIVYSGSTYYAAS | 1172 | HGGESDV | 1212 |
| | | | VKG | | | |
| 149362 | SSYYYW | 1133 | SIYYSGSAYYNPS | 1173 | HWQEWPDAFDI | 1213 |
| | G | | LKS | | | |
| 149363 | TSGMCV | 1134 | RIDWDEDKFYSTS | 1174 | SGAGGTSATAF | 1214 |
| | S | | LKT | | DI | |
| 149364 | SYSMN | 1135 | SISSSSSYIYYADS | 1175 | TIAAVYAFDI | 1215 |
| | | | VKG | | | |
| 149365 | DYYMS | 1136 | YISSSGSTIYYADS | 1176 | DLRGAFDI | 1216 |
| | | | VKG | | | |
| 149366 | SHYIH | 1137 | MINPSGGVTAYSQ | 1177 | EGSGSGWYFDF | 1217 |
| | | | TLQG | | | |
| 149367 | SGGYY | 1138 | YIYYSGSTYYNPS | 1178 | AGIAARLRGAF | 1218 |
| | WS | | LKS | | DI | |
| 149368 | SYAIS | 1139 | GIIPIFGTANYAQK | 1179 | RGGYQLLRWD | 1219 |
| | | | FQG | | VGLLRSAFDI | |
| 149369 | SNSAAW | 1140 | RTYYRSKWYSFY | 1180 | SSPEGLFLYWF | 1220 |
| | N | | AISLKS | | DP | |
| BCMA EB | SYAMS | 1141 | AISGSGGSTYYAD | 1181 | VEGSGSLDY | 1221 |
| B-C1978- | | | SVKG | | | |
| A4 | | | | | | |
| BCMA EB | RYPMS | 1142 | GISDSGVSTYYAD | 1182 | RAGSEASDI | 1222 |
| B-C1978- | | | SAKG | | | |
| G1 | | | | | | |
| BCMA EB | SYAMS | 1143 | AISGSGGSTYYAD | 1183 | ATYKRELRYYY | 1223 |
| B-C1979- | | | SVKG | | GMDV | |
| C1 | | | | | | |
| BCMA EB | SYAMS | 1144 | AISGSGGSTYYAD | 1184 | ATYKRELRYYY | 1224 |
| B-C1978- | | | SVKG | | GMDV | |
| C7 | | | | | | |
| BCMA EB | DYAMH | 1145 | GISWNSGSIGYAD | 1185 | VGKAVPDV | 1225 |
| B-C1978- | | | SVKG | | | |
| D10 | | | | | | |
| BCMA EB | DYAMH | 1146 | SINWKGNSLAYG | 1186 | HQGVAYYNYA | 1226 |
| B-C1979- | | | DSVKG | | MDV | |
| C12 | | | | | | |
| BCMA EB | SYAMS | 1147 | AISGSGGSTYYAD | 1187 | VVRDGMDV | 1227 |
| B-C1980- | | | SVKG | | | |
| G4 | | | | | | |
| BCMA EB | SYAMS | 1148 | AISGSGGSTYYAD | 1188 | IPQTGTFDY | 1228 |
| B-C1980- | | | SVKG | | | |
| D2 | | | | | | |
| BCMA EB | SYAMS | 1149 | AISGSGGSTYYAD | 1189 | ANYKRELRYYY | 1229 |
| B-C1978- | | | SVKG | | GMDV | |
| A10 | | | | | | |
| BCMA EB | SYAMS | 1150 | AISGSGGSTYYAD | 1190 | ALVGATGAFDI | 1230 |
| B-C1978- | | | SVKG | | | |
| D4 | | | | | | |
| BCMA EB | SYAMS | 1151 | AISGSGGSTYYAD | 1191 | WFGEGFDP | 1231 |
| B-C1980- | | | SVKG | | | |
| A2 | | | | | | |
| BCMA EB | SYAMS | 1152 | AISGSGGSTYYAD | 1192 | VGYDSSGYYRD | 1232 |
| B-C1981- | | | SVKG | | YYGMDV | |
| C3 | | | | | | |
| BCMA EB | SYAMS | 1153 | AISGSGGSTYYAD | 1193 | MGWSSGYLGA | 1233 |
| B-C1978- | | | SVKG | | FDI | |
| G4 | | | | | | |
| A7D12.2 | NFGMN | 1154 | WINTYTGESYFAD | 1194 | GEIYYGYDGGF | 1234 |
| | | | DFKG | | AY | |
| C11D5.3 | DYSIN | 1155 | WINTETREPAYAY | 1195 | DYSYAMDY | 1235 |
| | | | DFRG | | | |
| C12A3.2 | HYSMN | 1156 | RINTESGVPIYADD | 1196 | DYLYSLDF | 1236 |
| | | | FKG | | | |
| C13F12.1 | HYSMN | 1157 | RINTETGEPLYAD | 1197 | DYLYSCDY | 1237 |
| | | | DFKG | | | |

TABLE 8
Exemplary Light Chain Variable Domain CDRs according to the Kabat numbering
scheme (Kabat et al. (1991), “Sequences of Proteins of Immunological
Interest,” 5th Ed. Public Health Service, National Institutes
of Health, Bethesda, MD)

| | | SEQ | | SEQ | | SEQ |
|---|---|---|---|---|---|---|
| | | ID | | ID | | ID |
| Candidate | LCDR1 | NO | LCDR2 | NO | LCDR3 | NO |
| 139109 | RASQSISSYLN | 1238 | AASSLQS | 1278 | QQSYSTPYT | 1318 |
| 139103 | RASQSISSSFLA | 1239 | GASRRAT | 1279 | QQYHSSPSWT | 1319 |
| 139105 | RSSQSLLHSNGYNY | 1240 | LGSNRAS | 1280 | MQALQTPYT | 1320 |
| | LD | | | | | |
| 139111 | KSSQSLLRNDGKTP | 1241 | EVSNRFS | 1281 | MQNIQFPS | 1321 |
| | LY | | | | | |
| 139100 | RSSQSLLHSNGYNY | 1242 | LGSKRAS | 1282 | MQALQTPYT | 1322 |
| | LN | | | | | |
| 139101 | RASQSISSYLN | 1243 | GASTLAS | 1283 | QQSYKRAS | 1323 |
| 139102 | RSSQSLLYSNGYNY | 1244 | LGSNRAS | 1284 | MQGRQFPYS | 1324 |
| | VD | | | | | |
| 139104 | RASQSVSSNLA | 1245 | GASTRAS | 1285 | QQYGSSLT | 1325 |
| 139106 | RASQSVSSKLA | 1246 | GASIRAT | 1286 | QQYGSSSWT | 1326 |
| 139107 | RASQSVGSTNLA | 1247 | DASNRAT | 1287 | QQYGSSPPWT | 1327 |
| 139108 | RASQSISSYLN | 1248 | AASSLQS | 1288 | QQSYTLA | 1328 |
| 139110 | KSSESLVHNSGKTY | 1249 | EVSNRDS | 1289 | MQGTHWPGT | 1329 |
| | LN | | | | | |
| 139112 | QASEDINKFLN | 1250 | DASTLQT | 1290 | QQYESLPLT | 1330 |
| 139113 | RASQSVGSNLA | 1251 | GASTRAT | 1291 | QQYNDWLPV | 1331 |
| | | | | | T | |
| 139114 | RASQSIGSSSLA | 1252 | GASSRAS | 1292 | QQYAGSPPFT | 1332 |
| 149362 | KASQDIDDAMN | 1253 | SATSPVP | 1293 | LQHDNFPLT | 1333 |
| 149363 | RASQDIYNNLA | 1254 | AANKSQS | 1294 | QHYYRFPYS | 1334 |
| 149364 | RSSQSLLHSNGY | 1255 | LGSNRAS | 1295 | MQALQTPYT | 1335 |
| | NYLD | | | | | |
| 149365 | GGNNIGTKSVH | 1256 | DDSVRPS | 1296 | QVWDSDSEH | 1336 |
| | | | | | VV | |
| 149366 | SGDGLSKKYVS | 1257 | RDKERPS | 1297 | QAWDDTTVV | 1337 |
| 149367 | RASQGIRNWLA | 1258 | AASNLQS | 1298 | QKYNSAPFT | 1338 |
| 149368 | GGNNIGSKSVH | 1259 | GKNNRPS | 1299 | SSRDSSGDHL | 1339 |
| | | | | | RV | |
| 149369 | QGDSLGNYYAT | 1260 | GTNNRPS | 1300 | NSRDSSGHHL | 1340 |
| | | | | | L | |
| BCMA EBB | RASQSVSSAYLA | 1261 | GASTRAT | 1301 | QHYGSSFNGS | 1341 |
| -C1978-A4 | | | | | SLFT | |
| BCMA EBB | RASQSVSNSLA | 1262 | DASSRAT | 1302 | QQFGTSSGLT | 1342 |
| -C1978-G1 | | | | | | |
| BCMA EBB | RASQSVSSSFLA | 1263 | GASSRAT | 1303 | QQYHSSPSWT | 1343 |
| -C1979-C1 | | | | | | |
| BCMA EBB | RASQSVSTTFLA | 1264 | GSSNRAT | 1304 | QQYHSSPSWT | 1344 |
| -C1978-C7 | | | | | | |
| BCMA EBB | RASQSISSYLN | 1265 | AASSLQS | 1305 | QQSYSTPYS | 1345 |
| -C1978-D10 | | | | | | |
| BCMA EBB | RATQSIGSSFLA | 1266 | GASQRAT | 1306 | QHYESSPSWT | 1346 |
| -C1979-C12 | | | | | | |
| BCMA EBB | RASQSVSSSYLA | 1267 | GASSRAT | 1307 | QQYGSPPRFT | 1347 |
| -C1980-G4 | | | | | | |
| BCMA EBB | RASQSVSSSYLA | 1268 | GASSRAT | 1308 | QHYGSSPSWT | 1348 |
| -C1980-D2 | | | | | | |
| BCMA EBB | RASQRVASNYLA | 1269 | GASSRAT | 1309 | QHYDSSPSWT | 1349 |
| -C1978-A10 | | | | | | |
| BCMA EBB | RASQSLSSNFLA | 1270 | GASNWAT | 1310 | QYYGTSPMY | 1350 |
| C1978-D4 | | | | | T | |
| BCMA EBB | RSSQSLLHSNGYNY | 1271 | LGSNRAS | 1311 | MQALQTPLT | 1351 |
| -C1980-A2 | LD | | | | | |
| BCMA EBB | RASQSVSSSYLA | 1272 | GTSSRAT | 1312 | QHYGNSPPKF | 1352 |
| -C1981-C3 | | | | | T | |
| BCMA EBB | RASQSVASSFLA | 1273 | GASGRAT | 1313 | QHYGGSPRLT | 1353 |
| -C1978-G4 | | | | | | |
| A7D12.2 | RASQDVNTAVS | 1274 | SASYRYT | 1314 | QQHYSTPWT | 1354 |
| C11D5.3 | RASESVSVIGAHLI | 1275 | LASNLET | 1315 | LQSRIFPRT | 1355 |
| | H | | | | | |
| C12A3.2 | RASESVTILGSHLIY | 1276 | LASNVQT | 1316 | LQSRTIPRT | 1356 |
| C13F12.1 | RASESVTILGSHLIY | 1277 | LASNVQT | 1317 | LQSRTIPRT | 1357 |

### CD20 CAR and CD20-Binding Sequences

In some embodiments, the CAR or CAR-expressing cell described herein is a CD20 CAR-expressing cell (e.g., a cell expressing a CAR molecule that binds to human CD20). In some embodiments, the CD20 CAR-expressing cell includes an antigen-binding domain according to WO2016/164731 and PCT/US2017/055627, incorporated herein by reference. Exemplary CD20-binding sequences or CD20 CAR sequences are disclosed in, e.g., Tables 1-5 of PCT/US2017/055627. In some embodiments, the CD20-binding sequences or CD20 CAR comprises a CDR, variable region, scFv, or full-length sequence of a CD20 CAR disclosed in PCT/US2017/055627 or WO2016/164731.

In some embodiments, the CAR molecule comprises an antigen-binding domain that binds specifically to CD20 (CD20 CAR). In some embodiments, the antigen-binding domain targets human CD20. In some embodiments, the antigen-binding domain includes a single chain Fv sequence as described herein. The sequences of human CD20 CAR are provided below.

TABLE 9
Exemplary CD20 CAR Constructs

| SEQ ID | | |
|---|---|---|
| NUMBER | Ab region | Sequence |
| CD20-C3H2 | | |
| SEQ ID NO: | HCDR1 | NYNLH |
| 2019 (Kabat) | | |
| SEQ ID NO: | HCDR2 | AIYPGNYDTSYNQKFKG |
| 2020 (Kabat) | | |
| SEQ ID NO: | HCDR3 | VDFGHSRYWYFDV |
| 2021 (Kabat) | | |
| SEQ ID NO: | HCDR1 | GYTFTNY |
| 2022 (Chothia) | | |
| SEQ ID NO: | HCDR2 | YPGNYD |
| 2023 (Chothia) | | |
| SEQ ID NO: | HCDR3 | VDFGHSRYWYFDV |
| 2021 (Chothia) | | |
| SEQ ID NO: | HCDR1 | GYTFTNYN |
| 2024 (IMGT) | | |
| SEQ ID NO: | HCDR2 | IYPGNYDT |
| 2025 (IMGT) | | |
| SEQ ID NO: | HCDR3 | ARVDFGHSRYWYFDV |
| 2026 (IMGT) | | |
| SEQ ID NO: | HCDR1 | GYTFTNYNLH |
| 2027 (Combined | | |
| Chothia and | | |
| Kabat) | | |
| SEQ ID NO: | HCDR2 | AIYPGNYDTSYNQKFKG |
| 2020 (Combined | | |
| Chothia and | | |
| Kabat) | | |
| SEQ ID NO: | HCDR3 | VDFGHSRYWYFDV |
| 2021 (Combined | | |
| Chothia and | | |
| Kabat) | | |
| SEQ ID NO: | VH | QVQLVQSGAEVKKPGASVKVSCKASGYTFTNYNLHWV |
| 2028 | | RQAPGQGLEWMGAIYPGNYDTSYNQKFKGRVTMTAD |
| | | KSTSTAYMELSSLRSEDTAVYYCARVDFGHSRYWYFD |
| | | VWGQGTTVTVSS |
| SEQ ID NO: | DNA VH | CAAGTCCAACTCGTCCAGTCCGGTGCAGAAGTCAAG |
| 2029 | | AAACCTGGAGCATCCGTGAAAGTGTCTTGCAAAGCCT |
| | | CCGGCTACACCTTCACCAACTACAACCTCCATTGGGT |
| | | CAGACAGGCCCCCGGACAAGGACTCGAATGGATGGG |
| | | AGCGATCTACCCGGGAAACTACGACACCAGCTACAA |
| | | CCAGAAGTTCAAGGGCCGCGTGACTATGACCGCCGA |
| | | TAAGAGCACCTCCACCGCCTACATGGAACTGTCCTCG |
| | | CTGAGGTCCGAGGACACTGCGGTGTACTACTGCGCCC |
| | | GCGTGGACTTCGGACACTCACGGTATTGGTACTTCGA |
| | | CGTCTGGGGACAGGGCACTACCGTGACCGTGTCGAG |
| | | C |
| SEQ ID NO: | LCDR1 | RATSSVSSMN |
| 2030 (Kabat) | | |
| SEQ ID NO: | LCDR2 | ATSNLAS |
| 2031 (Kabat) | | |
| SEQ ID NO: | LCDR3 | QQWTFNPPT |
| 2032 (Kabat) | | |
| SEQ ID NO: | LCDR1 | TSSVSS |
| 2033 (Chothia) | | |
| SEQ ID NO: | LCDR2 | ATS |
| 2034 (Chothia) | | |
| SEQ ID NO: | LCDR3 | WTFNPP |
| 2035 (Chothia) | | |
| SEQ ID NO: | LCDR1 | SSVSS |
| 2036 (IMGT) | | |
| SEQ ID NO: | LCDR2 | ATS |
| 2034 (IMGT) | | |
| SEQ ID NO: | LCDR3 | QQWTFNPPT |
| 2032 (IMGT) | | |
| SEQ ID NO | LCDR1 | RATSSVSSMN |
| 2030 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWTFNPPT |
| 2032 (Combined | | |
| Chothia and | | |
| Kabat) | | |
| SEQ ID NO: | VL | DIQLTQSPSFLSASVGDRVTITCRATSSVSSMNWYQQKP |
| 2037 | | GKAPKPLIHATSNLASGVPSRFSGSGSGTEYTLTISSLQP |
| | | EDFATYYCQQWTFNPPTFGQGTKLEIK |
| SEQ ID NO: | DNA VL | GATATCCAGCTGACTCAGTCCCCGTCATTCCTGTCCG |
| 2038 | | CCTCCGTGGGAGACAGAGTGACCATCACCTGTCGGG |
| | | CCACTTCCTCCGTGTCAAGCATGAACTGGTATCAGCA |
| | | GAAGCCCGGGAAGGCCCCAAAGCCGCTGATTCACGC |
| | | GACGTCCAACCTGGCTTCCGGCGTGCCGAGCCGGTTC |
| | | TCCGGCTCGGGGAGCGGGACTGAGTACACCCTGACT |
| | | ATTTCCTCGCTTCAACCCGAGGACTTTGCTACCTACT |
| | | ACTGCCAACAGTGGACCTTCAATCCTCCGACATTCGG |
| | | ACAGGGTACCAAGTIGGAAATCAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTNYNLHWV |
| 2039 | linker-VL) | RQAPGQGLEWMGAIYPGNYDTSYNQKFKGRVTMTAD |
| | | KSTSTAYMELSSLRSEDTAVYYCARVDFGHSRYWYFD |
| | | VWGQGTTVTVSSGGGGSGGGGSGGGGSGGGGSDIQLT |
| | | QSPSFLSASVGDRVTITCRATSSVSSMNWYQQKPGKAP |
| | | KPLIHATSNLASGVPSRFSGSGSGTEYTLTISSLQPED |
| | | FATYYCQQWTFNPPTFGQGTKLEIK |
| SEQ ID NO: | DNA scFv | CAAGTCCAACTCGTCCAGTCCGGTGCAGAAGTCAAG |
| 2040 | (VH-linker- | AAACCTGGAGCATCCGTGAAAGTGTCTTGCAAAGCCT |
| | VL) | CCGGCTACACCTTCACCAACTACAACCTCCATTGGGT |
| | | CAGACAGGCCCCCGGACAAGGACTCGAATGGATGGG |
| | | AGCGATCTACCCGGGAAACTACGACACCAGCTACAA |
| | | CCAGAAGTTCAAGGGCCGCGTGACTATGACCGCCGA |
| | | TAAGAGCACCTCCACCGCCTACATGGAACTGTCCTCG |
| | | CTGAGGTCCGAGGACACTGCGGTGTACTACTGCGCCC |
| | | GCGTGGACTTCGGACACTCACGGTATTGGTACTTCGA |
| | | CGTCTGGGGACAGGGCACTACCGTGACCGTGTCGAG |
| | | CGGCGGAGGAGGTTCGGGAGGGGGCGGATCAGGGG |
| | | GCGGCGGCAGCGGTGGAGGGGGCTCGGATATCCAGC |
| | | TGACTCAGTCCCCGTCATTCCTGTCCGCCTCCGTGGG |
| | | AGACAGAGTGACCATCACCTGTCGGGCCACTTCCTCC |
| | | GTGTCAAGCATGAACTGGTATCAGCAGAAGCCCGGG |
| | | AAGGCCCCAAAGCCGCTGATTCACGCGACGTCCAAC |
| | | CTGGCTTCCGGCGTGCCGAGCCGGTTCTCCGGCTCGG |
| | | GGAGCGGGACTGAGTACACCCTGACTATTTCCTCGCT |
| | | TCAACCCGAGGACTTTGCTACCTACTACTGCCAACAG |
| | | TGGACCTTCAATCCTCCGACATTCGGACAGGGTACCA |
| | | AGTTGGAAATCAAG |
| SEQ ID NO: | Full CAR | MALPVTALLLPLALLLHAARPQVQLVQSGAEVKKPGAS |
| 2041 | amino acid | VKVSCKASGYTFTNYNLHWVRQAPGQGLEWMGAIYP |
| | sequence | GNYDTSYNQKFKGRVTMTADKSTSTAYMELSSLRSED |
| | | TAVYYCARVDFGHSRYWYFDVWGQGTTVTVSSGGGG |
| | | SGGGGSGGGGSGGGGSDIQLTQSPSFLSASVGDRVTITC |
| | | RATSSVSSMNWYQQKPGKAPKPLIHATSNLASGVPSRF |
| | | SGSGSGTEYTLTISSLQPEDFATYYCQQWTFNPPTFGQG |
| | | TKLEIKTTTPAPRPPTPAPTIASQPLSLRPEACRPAAGGA |
| | | VHTRGLDFACDIYIWAPLAGTCGVLLLSLVITLYCKRGR |
| | | KKLLYIFKQPFMRPVQTTQEEDGCSCRFPEEEEGGCELR |
| | | VKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPP |
| | | R |
| SEQ ID NO: | Full CAR | ATGGCCCTCCCTGTCACCGCCCTGCTGCTTCCGCTGG |
| 2042 | nucleic acid | CTCTTCTGCTCCACGCCGCTCGGCCCCAAGTCCAACT |
| | sequence | CGTCCAGTCCGGTGCAGAAGTCAAGAAACCTGGAGC |
| | | ATCCGTGAAAGTGTCTTGCAAAGCCTCCGGCTACACC |
| | | TTCACCAACTACAACCTCCATTGGGTCAGACAGGCCC |
| | | CCGGACAAGGACTCGAATGGATGGGAGCGATCTACC |
| | | CGGGAAACTACGACACCAGCTACAACCAGAAGTTCA |
| | | AGGGCCGCGTGACTATGACCGCCGATAAGAGCACCT |
| | | CCACCGCCTACATGGAACTGTCCTCGCTGAGGTCCGA |
| | | GGACACTGCGGTGTACTACTGCGCCCGCGTGGACTTC |
| | | GGACACTCACGGTATTGGTACTTCGACGTCTGGGGAC |
| | | AGGGCACTACCGTGACCGTGTCGAGCGGCGGAGGAG |
| | | GTTCGGGAGGGGGCGGATCAGGGGGCGGCGGCAGCG |
| | | GTGGAGGGGGCTCGGATATCCAGCTGACTCAGTCCCC |
| | | GTCATTCCTGTCCGCCTCCGTGGGAGACAGAGTGACC |
| | | ATCACCTGTCGGGCCACTTCCTCCGTGTCAAGCATGA |
| | | ACTGGTATCAGCAGAAGCCCGGGAAGGCCCCAAAGC |
| | | CGCTGATTCACGCGACGTCCAACCTGGCTTCCGGCGT |
| | | GCCGAGCCGGTTCTCCGGCTCGGGGAGCGGGACTGA |
| | | GTACACCCTGACTATTTCCTCGCTTCAACCCGAGGAC |
| | | TTTGCTACCTACTACTGCCAACAGTGGACCTTCAATC |
| | | CTCCGACATTCGGACAGGGTACCAAGTTGGAAATCA |
| | | AGACCACTACCCCAGCACCGAGGCCACCCACCCCGG |
| | | CTCCTACCATCGCCTCCCAGCCTCTGTCCCTGCGTCCG |
| | | GAGGCATGTAGACCCGCAGCTGGTGGGGCCGTGCAT |
| | | ACCCGGGGTCTTGACTTCGCCTGCGATATCTACATTT |
| | | GGGCCCCTCTGGCTGGTACTTGCGGGGTCCTGCTGCT |
| | | TTCACTCGTGATCACTCTTTACTGTAAGCGCGGTCGG |
| | | AAGAAGCTGCTGTACATCTTTAAGCAACCCTTCATGA |
| | | GGCCTGTGCAGACTACTCAAGAGGAGGACGGCTGTT |
| | | CATGCCGGTTCCCAGAGGAGGAGGAAGGCGGCTGCG |
| | | AACTGCGCGTGAAATTCAGCCGCAGCGCAGATGCTC |
| | | CAGCCTACCAGCAGGGGCAGAACCAGCTCTACAACG |
| | | AACTCAATCTTGGTCGGAGAGAGGAGTACGACGTGC |
| | | TGGACAAGCGGAGAGGACGGGACCCAGAAATGGGC |
| | | GGGAAGCCGCGCAGAAAGAATCCCCAAGAGGGCCTG |
| | | TACAACGAGCTCCAAAAGGATAAGATGGCAGAAGCC |
| | | TATAGCGAGATTGGTATGAAAGGGGAACGCAGAAGA |
| | | GGCAAAGGCCACGACGGACTGTACCAGGGACTCAGC |
| | | ACCGCCACCAAGGACACCTATGACGCTCTTCACATGC |
| | | AGGCCCTGCCGCCTCGG |
| CD20-C5H1 | | |
| SEQ ID NO: | HCDR1 | SYNMH |
| 2043 (Kabat) | | |
| SEQ ID NO: | HCDR2 | AIYPGNGDTSYNPKFKG |
| 2044 (Kabat) | | |
| SEQ ID NO: | HCDR3 | SYFYGSSSWYFDV |
| 2045 (Kabat) | | |
| SEQ ID NO: | HCDR1 | GYTFTSY |
| 2046 (Chothia) | | |
| SEQ ID NO: | HCDR2 | YPGNGD |
| 2047 (Chothia) | | |
| SEQ ID NO: | HCDR3 | SYFYGSSSWYFDV |
| 2045 (Chothia) | | |
| SEQ ID NO: | HCDR1 | GYTFTSYN |
| 2048 (IMGT) | | |
| SEQ ID NO: | HCDR2 | IYPGNGDT |
| 2049 (IMGT) | | |
| SEQ ID NO: | HCDR3 | ARSYFYGSSSWYFDV |
| 2050 (IMGT) | | |
| SEQ ID NO: | HCDR1 | GYTFTSYNMH |
| 2051 (Combined | | |
| Chothia and | | |
| Kabat) | | |
| SEQ ID NO: | HCDR2 | AIYPGNGDTSYNPKFKG |
| 2044 (Combined | | |
| Chothia and | | |
| Kabat | | |
| SEQ ID NO: | HCDR3 | SYFYGSSSWYFDV |
| 2045 (Combined | | |
| Chothia and | | |
| Kabat) | | |
| SEQ ID NO: | VH | QVQLVQSGAEVKKPGASVKVSCKASGYTFTSYNMHW |
| 2052 | | VRQAPGQGLEWMGAIYPGNGDTSYNPKFKGRVTMTA |
| | | DKSTRTAYMELSSLRSEDTAVYYCARSYFYGSSSWYFD |
| | | VWGQGTTVTVSS |
| SEQ ID NO: | DNA VH | CAAGTGCAGCTCGTCCAGTCCGGTGCAGAAGTCAAG |
| 2053 | | AAACCCGGTGCTTCAGTGAAAGTGTCCTGCAAGGCCT |
| | | CCGGTTACACCTTCACCTCCTACAACATGCACTGGGT |
| | | CCGCCAAGCCCCGGGCCAGGGACTCGAATGGATGGG |
| | | AGCCATCTACCCTGGCAACGGGGACACCTCATACAA |
| | | CCCTAAGTTCAAGGGCAGAGTGACCATGACTGCGGA |
| | | CAAGTCCACTAGAACAGCGTACATGGAGCTGAGCAG |
| | | CCTGCGGTCCGAGGATACTGCCGTGTACTACTGCGCC |
| | | CGCTCCTACTTCTACGGAAGCTCGTCGTGGTACTTCG |
| | | ATGTCTGGGGACAGGGCACCACTGTGACTGTGTCCTC |
| | | C |
| SEQ ID NO: | LCDR1 | RASSSVSSMH |
| 2054 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDR1 | SSSVSS |
| 2056 (Chothia) | | |
| SEQ ID NO: | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVSS |
| 2036 (IMGT) | | |
| SEQ ID NO. | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVSSMH |
| 2054 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 | | |
| (Combined | | |
| Chothia and | | |
| Kabat) | | |
| SEQ ID NO: | VL | EIVLTQSPATLSLSPGERATLSCRASSSVSSMHWYQQKP |
| 2058 | | GQAPRPLIFATSNLASGIPARFSGSGSGTDYTLTISSLEPE |
| | | DAAVYYCQQWIFNPPTFGGGTKVEIK |
| SEQ ID NO: | DNA VL | GAAATTGTGCTGACTCAGAGCCCCGCCACCCTGAGCT |
| 2059 | | TGTCCCCCGGGGAAAGGGCAACGCTGTCATGCCGCG |
| | | CCTCGTCATCCGTGTCCTCCATGCATTGGTACCAGCA |
| | | GAAGCCGGGACAGGCCCCTCGGCCGCTGATCTTCGCC |
| | | ACCTCCAATCTCGCTTCCGGCATTCCGGCCCGGTTCT |
| | | CGGGAAGCGGGTCGGGGACCGACTATACCCTGACCA |
| | | TCTCTAGCCTTGAACCTGAGGACGCCGCGGTGTACTA |
| | | TTGTCAACAGTGGATCTTTAACCCCCCAACCTTCGGT |
| | | GGAGGCACCAAAGTGGAGATTAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTSYNMHW |
| 2060 | linker-VL) | VRQAPGQGLEWMGAIYPGNGDTSYNPKFKGRVTMTA |
| | | DKSTRTAYMELSSLRSEDTAVYYCARSYFYGSSSWYFD |
| | | VWGQGTTVTVSSGGGGSGGGGSGGGGSGGGGSEIVLT |
| | | QSPATLSLSPGERATLSCRASSSVSSMHWYQQKPGQAP |
| | | RPLIFATSNLASGIPARFSGSGSGTDYTLTISSLEPED |
| | | AAVYYCQQWIFNPPTFGGGTKVEIK |
| SEQ ID NO: | DNA scFv | CAAGTGCAGCTCGTCCAGTCCGGTGCAGAAGTCAAG |
| 2061 | (VH-linker- | AAACCCGGTGCTTCAGTGAAAGTGTCCTGCAAGGCCT |
| | VL) | CCGGTTACACCTTCACCTCCTACAACATGCACTGGGT |
| | | CCGCCAAGCCCCGGGCCAGGGACTCGAATGGATGGG |
| | | AGCCATCTACCCTGGCAACGGGGACACCTCATACAA |
| | | CCCTAAGTTCAAGGGCAGAGTGACCATGACTGCGGA |
| | | CAAGTCCACTAGAACAGCGTACATGGAGCTGAGCAG |
| | | CCTGCGGTCCGAGGATACTGCCGTGTACTACTGCGCC |
| | | CGCTCCTACTTCTACGGAAGCTCGTCGTGGTACTTCG |
| | | ATGTCTGGGGACAGGGCACCACTGTGACTGTGTCCTC |
| | | CGGTGGCGGAGGCTCGGGCGGAGGCGGAAGCGGCGG |
| | | CGGGGGATCGGGAGGAGGAGGGTCCGAAATTGTGCT |
| | | GACTCAGAGCCCCGCCACCCTGAGCTTGTCCCCCGGG |
| | | GAAAGGGCAACGCTGTCATGCCGCGCCTCGTCATCCG |
| | | TGTCCTCCATGCATTGGTACCAGCAGAAGCCGGGACA |
| | | GGCCCCTCGGCCGCTGATCTTCGCCACCTCCAATCTC |
| | | GCTTCCGGCATTCCGGCCCGGTTCTCGGGAAGCGGGT |
| | | CGGGGACCGACTATACCCTGACCATCTCTAGCCTTGA |
| | | ACCTGAGGACGCCGCGGTGTACTATTGTCAACAGTGG |
| | | ATCTTTAACCCCCCAACCTTCGGTGGAGGCACCAAAG |
| | | TGGAGATTAAG |
| SEQ ID NO: | Full CAR | MALPVTALLLPLALLLHAARPQVQLVQSGAEVKKPGAS |
| 2062 | amino acid | VKVSCKASGYTFTSYNMHWVRQAPGQGLEWMGAIYP |
| | | GNGDTSYNPKFKGRVTMTADKSTRTAYMELSSLRSEDT |
| | | AVYYCARSYFYGSSSWYFDVWGQGTTVTVSSGGGGSG |
| | | GGGSGGGGSGGGGSEIVLTQSPATLSLSPGERATLSCRA |
| | | SSSVSSMHWYQQKPGQAPRPLIFATSNLASGIPARFSGS |
| | | GSGTDYTLTISSLEPEDAAVYYCQQWIFNPPTFGGGTKV |
| | | EIKTTTPAPRPPTPAPTIASQPLSLRPEACRPAAGGAVHT |
| | | RGLDFACDIYIWAPLAGTCGVLLLSLVITLYCKRGRKKL |
| | | LYIFKQPFMRPVQTTQEEDGCSCRFPEEEEGGCELRVKF |
| | | SRSADAPAYQQGQNQLYNELNLGRREEYDVLDKRRGR |
| | | DPEMGGKPRRKNPQEGLYNELQKDKMAEAYSEIGMKG |
| | | ERRRGKGHDGLYQGLSTATKDTYDALHMQALPPR |
| SEQ ID NO: | sequence | ATGGCCCTCCCTGTCACCGCCCTGCTGCTTCCGCTGG |
| 2063 | Full CAR | CTCTTCTGCTCCACGCCGCTCGGCCCCAAGTGCAGCT |
| | nucleic acid | CGTCCAGTCCGGTGCAGAAGTCAAGAAACCCGGTGC |
| | sequence | TTCAGTGAAAGTGTCCTGCAAGGCCTCCGGTTACACC |
| | | TTCACCTCCTACAACATGCACTGGGTCCGCCAAGCCC |
| | | CGGGCCAGGGACTCGAATGGATGGGAGCCATCTACC |
| | | CTGGCAACGGGGACACCTCATACAACCCTAAGTTCA |
| | | AGGGCAGAGTGACCATGACTGCGGACAAGTCCACTA |
| | | GAACAGCGTACATGGAGCTGAGCAGCCTGCGGTCCG |
| | | AGGATACTGCCGTGTACTACTGCGCCCGCTCCTACTT |
| | | CTACGGAAGCTCGTCGTGGTACTTCGATGTCTGGGGA |
| | | CAGGGCACCACTGTGACTGTGTCCTCCGGTGGCGGAG |
| | | GCTCGGGCGGAGGCGGAAGCGGCGGCGGGGGATCGG |
| | | GAGGAGGAGGGTCCGAAATTGTGCTGACTCAGAGCC |
| | | CCGCCACCCTGAGCTTGTCCCCCGGGGAAAGGGCAA |
| | | CGCTGTCATGCCGCGCCTCGTCATCCGTGTCCTCCAT |
| | | GCATTGGTACCAGCAGAAGCCGGGACAGGCCCCTCG |
| | | GCCGCTGATCTTCGCCACCTCCAATCTCGCTTCCGGC |
| | | ATTCCGGCCCGGTTCTCGGGAAGCGGGTCGGGGACC |
| | | GACTATACCCTGACCATCTCTAGCCTTGAACCTGAGG |
| | | ACGCCGCGGTGTACTATTGTCAACAGTGGATCTTTAA |
| | | CCCCCCAACCTTCGGTGGAGGCACCAAAGTGGAGAT |
| | | TAAGACCACTACCCCAGCACCGAGGCCACCCACCCC |
| | | GGCTCCTACCATCGCCTCCCAGCCTCTGTCCCTGCGT |
| | | CCGGAGGCATGTAGACCCGCAGCTGGTGGGGCCGTG |
| | | CATACCCGGGGTCTTGACTTCGCCTGCGATATCTACA |
| | | TTTGGGCCCCTCTGGCTGGTACTTGCGGGGTCCTGCT |
| | | GCTTTCACTCGTGATCACTCTTTACTGTAAGCGCGGT |
| | | CGGAAGAAGCTGCTGTACATCTTTAAGCAACCCTTCA |
| | | TGAGGCCTGTGCAGACTACTCAAGAGGAGGACGGCT |
| | | GTTCATGCCGGTTCCCAGAGGAGGAGGAAGGCGGCT |
| | | GCGAACTGCGCGTGAAATTCAGCCGCAGCGCAGATG |
| | | CTCCAGCCTACCAGCAGGGGCAGAACCAGCTCTACA |
| | | ACGAACTCAATCTTGGTCGGAGAGAGGAGTACGACG |
| | | TGCTGGACAAGCGGAGAGGACGGGACCCAGAAATGG |
| | | GCGGGAAGCCGCGCAGAAAGAATCCCCAAGAGGGCC |
| | | TGTACAACGAGCTCCAAAAGGATAAGATGGCAGAAG |
| | | CCTATAGCGAGATTGGTATGAAAGGGGAACGCAGAA |
| | | GAGGCAAAGGCCACGACGGACTGTACCAGGGACTCA |
| | | GCACCGCCACCAAGGACACCTATGACGCTCTTCACAT |
| | | GCAGGCCCTGCCGCCTCGG |
| CD20-C2H1 | | |
| SEQ ID NO: | HCDR1 | NYWMH |
| 2064 | | |
| (Kabat) | | |
| SEQ ID NO: | HCDR2 | FITPTTGYPEYNQKFKD |
| 2065 (Kabat) | | |
| SEQ ID NO: | HCDR3 | RKVGKGVYYALDY |
| 2066 (Kabat) | | |
| SEQ ID NO: | HCDR1 | GYTFTNY |
| 2022 (Chothia) | | |
| SEQ ID NO: | HCDR2 | TPTTGY |
| 2067 (Chothia) | | |
| SEQ ID NO: | HCDR3 | RKVGKGVYYALDY |
| 2066 (Chothia) | | |
| SEQ ID NO: | HCDR1 | GYTFTNYW |
| 2068 (IMGT) | | |
| SEQ ID NO: | HCDR2 | ITPTTGYP |
| 2069 (IMGT) | | |
| SEQ ID NO: | HCDR3 | ARRKVGKGVYYALDY |
| 2070 (IMGT) | | |
| SEQ ID NO: | HCDR1 | GYTFTNYWMH |
| 2071 (Combined | | |
| Chothia and | | |
| Kabat) | | |
| SEQ ID NO: | HCDR2 | FITPTTGYPEYNQKFKD |
| 2065 (Combined | | |
| Chothia and | | |
| Kabat) | | |
| SEQ ID NO: | HCDR3 | RKVGKGVYYALDY |
| 2066 (Combined | | |
| Chothia and | | |
| Kabat) | | |
| SEQ ID NO: | VH | QVQLVQSGAEVKKPGASVKVSCKASGYTFTNYWMHW |
| 2072 | | VRQAPGQGLEWMGFITPTTGYPEYNQKFKDRVTMTAD |
| | | KSTSTAYMELSSLRSEDTAVYYCARRKVGKGVYYALD |
| | | YWGQGTTVTVSS |
| SEQ ID NO: | DNA VH | CAAGTGCAACTCGTCCAGTCCGGTGCAGAAGTCAAG |
| 2073 | | AAACCAGGCGCATCCGTGAAAGTCTCCTGCAAAGCC |
| | | TCCGGCTACACATTCACTAACTATTGGATGCATTGGG |
| | | TGCGCCAGGCCCCGGGACAGGGGCTGGAGTGGATGG |
| | | GGTTCATTACCCCTACCACCGGCTACCCTGAGTACAA |
| | | CCAGAAGTTCAAGGATAGGGTCACCATGACCGCTGA |
| | | CAAGTCCACCTCCACCGCGTACATGGAACTGTCATCG |
| | | CTCCGGTCCGAGGATACCGCGGTGTACTACTGCGCCC |
| | | GGAGAAAAGTCGGAAAGGGAGTGTATTACGCCTTGG |
| | | ACTACTGGGGACAGGGGACTACCGTGACCGTGTCGA |
| | | GC |
| SEQ ID NO: | LCDR1 | RASGNIHNYLA |
| 2074 (Kabat) | | |
| SEQ ID NO: | LCDR2 | NTKTLAD |
| 2075 (Kabat) | | |
| SEQ ID NO: | LCDR3 | QHFWSSPWT |
| 2076 (Kabat) | | |
| SEQ ID NO: | LCDR1 | SGNIHNY |
| 2077 (Chothia) | | |
| SEQ ID NO: | LCDR2 | NTK |
| 2078 (Chothia) | | |
| SEQ ID NO: | LCDR3 | FWSSPW |
| 2079 (Chothia) | | |
| SEQ ID NO: | LCDR1 | GNIHNY |
| 2080 (IMGT) | | |
| SEQ ID NO: | LCDR2 | NTK |
| 2078 (IMGT) | | |
| SEQ ID NO: | LCDR3 | QHFWSSPWT |
| 2076 (IMGT) | | |
| SEQ ID NO: | LCDR1 | RASGNIHNYLA |
| 2074 (Combined | | |
| Chothia and | | |
| Kabat | | |
| SEQ ID NO: | LCDR2 | NTKTLAD |
| 2075 (Combined | | |
| Chothia and | | |
| Kabat) | | |
| SEQ ID NO: | LCDR3 | QHFWSSPWT |
| 2076 (Combined | | |
| Chothia and | | |
| Kabat | | |
| SEQ ID NO: | VL | DIQMTQSPSSLSASVGDRVTITCRASGNIHNYLAWYQQ |
| 2081 | | KPGKVPKLLIYNTKTLADGVPSRFSGSGSGTDYTLTIS |
| | | SLQPEDVATYYCQHFWSSPWTFGGGTKVEIK |
| SEQ ID NO: | DNA VL | GACATCCAGATGACCCAGTCCCCGTCAAGCCTTAGCG |
| 2082 | | CCTCCGTGGGCGACCGCGTGACCATTACTTGTCGGGC |
| | | GTCGGGAAACATCCACAACTACCTCGCCTGGTACCAG |
| | | CAGAAGCCGGGAAAGGTCCCCAAGCTGCTGATCTAC |
| | | AATACCAAGACTCTGGCCGACGGAGTGCCTTCCCGCT |
| | | TTTCCGGTTCGGGAAGCGGGACTGACTACACCCTGAC |
| | | TATCTCCTCGCTGCAACCCGAAGATGTGGCTACGTAC |
| | | TACTGCCAGCACTTCTGGTCCTCTCCCTGGACCTTCG |
| | | GCGGTGGCACTAAGGTCGAGATTAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTNYWMHW |
| 2083 | linker-VL) | VRQAPGQGLEWMGFITPTTGYPEYNQKFKDRVTMTAD |
| | | KSTSTAYMELSSLRSEDTAVYYCARRKVGKGVYYALD |
| | | YWGQGTTVTVSSGGGGSGGGGSGGGGSGGGGSDIQMT |
| | | QSPSSLSASVGDRVTITCRASGNIHNYLAWYQQKPGKV |
| | | PKLLIYNTKTLADGVPSRFSGSGSGTDYTLTISSLQPE |
| | | DVATYYCQHFWSSPWTFGGGTKVEIK |
| SEQ ID NO: | DNA scFv | CAAGTGCAACTCGTCCAGTCCGGTGCAGAAGTCAAG |
| 2084 | (VH-linker- | AAACCAGGCGCATCCGTGAAAGTCTCCTGCAAAGCC |
| | VL) | TCCGGCTACACATTCACTAACTATTGGATGCATTGGG |
| | | TGCGCCAGGCCCCGGGACAGGGGCTGGAGTGGATGG |
| | | GGTTCATTACCCCTACCACCGGCTACCCTGAGTACAA |
| | | CCAGAAGTTCAAGGATAGGGTCACCATGACCGCTGA |
| | | CAAGTCCACCTCCACCGCGTACATGGAACTGTCATCG |
| | | CTCCGGTCCGAGGATACCGCGGTGTACTACTGCGCCC |
| | | GGAGAAAAGTCGGAAAGGGAGTGTATTACGCCTTGG |
| | | ACTACTGGGGACAGGGGACTACCGTGACCGTGTCGA |
| | | GCGGTGGAGGCGGCTCCGGCGGAGGAGGAAGCGGG |
| | | GGAGGCGGTTCAGGGGGCGGAGGAAGCGACATCCAG |
| | | ATGACCCAGTCCCCGTCAAGCCTTAGCGCCTCCGTGG |
| | | GCGACCGCGTGACCATTACTTGTCGGGCGTCGGGAA |
| | | ACATCCACAACTACCTCGCCTGGTACCAGCAGAAGCC |
| | | GGGAAAGGTCCCCAAGCTGCTGATCTACAATACCAA |
| | | GACTCTGGCCGACGGAGTGCCTTCCCGCTTTTCCGGT |
| | | TCGGGAAGCGGGACTGACTACACCCTGACTATCTCCT |
| | | CGCTGCAACCCGAAGATGTGGCTACGTACTACTGCCA |
| | | GCACTTCTGGTCCTCTCCCTGGACCTTCGGCGGTGGC |
| | | ACTAAGGTCGAGATTAAG |
| SEQ ID NO: | Full CAR | MALPVTALLLPLALLLHAARPQVQLVQSGAEVKKPGAS |
| 2085 | amino acid | VKVSCKASGYTFTNYWMHWVRQAPGQGLEWMGFITP |
| | sequence | TTGYPEYNQKFKDRVTMTADKSTSTAYMELSSLRSEDT |
| | | AVYYCARRKVGKGVYYALDYWGQGTTVTVSSGGGGS |
| | | GGGGSGGGGSGGGGSDIQMTQSPSSLSASVGDRVTITC |
| | | RASGNIHNYLAWYQQKPGKVPKLLIYNTKTLADGVPSR |
| | | FSGSGSGTDYTLTISSLQPEDVATYYCQHFWSSPWTFGG |
| | | GTKVEIKTTTPAPRPPTPAPTIASQPLSLRPEACRPAAGG |
| | | AVHTRGLDFACDIYIWAPLAGTCGVLLLSLVITLYCKRG |
| | | RKKLLYIFKQPFMRPVQTTQEEDGCSCRFPEEEEGGCEL |
| | | RVKFSRSADAPAYQQGQNQLYNELNLGRREEYDVLDK |
| | | RRGRDPEMGGKPRRKNPQEGLYNELQKDKMAEAYSEI |
| | | GMKGERRRGKGHDGLYQGLSTATKDTYDALHMQALP |
| | | PR |
| SEQ ID NO: | Full CAR | ATGGCCCTCCCTGTCACCGCCCTGCTGCTTCCGCTGG |
| 2086 | nucleic acid | CTCTTCTGCTCCACGCCGCTCGGCCCCAAGTGCAACT |
| | sequence | CGTCCAGTCCGGTGCAGAAGTCAAGAAACCAGGCGC |
| | | ATCCGTGAAAGTCTCCTGCAAAGCCTCCGGCTACACA |
| | | TTCACTAACTATTGGATGCATTGGGTGCGCCAGGCCC |
| | | CGGGACAGGGGCTGGAGTGGATGGGGTTCATTACCC |
| | | CTACCACCGGCTACCCTGAGTACAACCAGAAGTTCAA |
| | | GGATAGGGTCACCATGACCGCTGACAAGTCCACCTCC |
| | | ACCGCGTACATGGAACTGTCATCGCTCCGGTCCGAGG |
| | | ATACCGCGGTGTACTACTGCGCCCGGAGAAAAGTCG |
| | | GAAAGGGAGTGTATTACGCCTTGGACTACTGGGGAC |
| | | AGGGGACTACCGTGACCGTGTCGAGCGGTGGAGGCG |
| | | GCTCCGGCGGAGGAGGAAGCGGGGGAGGCGGTTCAG |
| | | GGGGCGGAGGAAGCGACATCCAGATGACCCAGTCCC |
| | | CGTCAAGCCTTAGCGCCTCCGTGGGCGACCGCGTGAC |
| | | CATTACTTGTCGGGCGTCGGGAAACATCCACAACTAC |
| | | CTCGCCTGGTACCAGCAGAAGCCGGGAAAGGTCCCC |
| | | AAGCTGCTGATCTACAATACCAAGACTCTGGCCGACG |
| | | GAGTGCCTTCCCGCTTTTCCGGTTCGGGAAGCGGGAC |
| | | TGACTACACCCTGACTATCTCCTCGCTGCAACCCGAA |
| | | GATGTGGCTACGTACTACTGCCAGCACTTCTGGTCCT |
| | | CTCCCTGGACCTTCGGCGGTGGCACTAAGGTCGAGAT |
| | | TAAGACCACTACCCCAGCACCGAGGCCACCCACCCC |
| | | GGCTCCTACCATCGCCTCCCAGCCTCTGTCCCTGCGT |
| | | CCGGAGGCATGTAGACCCGCAGCTGGTGGGGCCGTG |
| | | CATACCCGGGGTCTTGACTTCGCCTGCGATATCTACA |
| | | TTTGGGCCCCTCTGGCTGGTACTTGCGGGGTCCTGCT |
| | | GCTTTCACTCGTGATCACTCTTTACTGTAAGCGCGGT |
| | | CGGAAGAAGCTGCTGTACATCTTTAAGCAACCCTTCA |
| | | TGAGGCCTGTGCAGACTACTCAAGAGGAGGACGGCT |
| | | GTTCATGCCGGTTCCCAGAGGAGGAGGAAGGCGGCT |
| | | GCGAACTGCGCGTGAAATTCAGCCGCAGCGCAGATG |
| | | CTCCAGCCTACCAGCAGGGGCAGAACCAGCTCTACA |
| | | ACGAACTCAATCTTGGTCGGAGAGAGGAGTACGACG |
| | | TGCTGGACAAGCGGAGAGGACGGGACCCAGAAATGG |
| | | GCGGGAAGCCGCGCAGAAAGAATCCCCAAGAGGGCC |
| | | TGTACAACGAGCTCCAAAAGGATAAGATGGCAGAAG |
| | | CCTATAGCGAGATTGGTATGAAAGGGGAACGCAGAA |
| | | GAGGCAAAGGCCACGACGGACTGTACCAGGGACTCA |
| | | GCACCGCCACCAAGGACACCTATGACGCTCTTCACAT |
| | | GCAGGCCCTGCCGCCTCGG |
| CD20-C2H2 | | |
| SEQ ID NO: | HCDR1 | NYWMH |
| 2064 (Kabat) | | |
| SEQ ID NO: | HCDR2 | FITPTTGYPEYNQKFKD |
| 2065 (Kabat) | | |
| SEQ ID NO: | HCDR3 | RKVGKGVYYALDY |
| 2066 (Kabat) | | |
| SEQ ID NO: | HCDR1 | GYTFTNY |
| 2022 (Chothia) | | |
| SEQ ID NO: | HCDR2 | TPTTGY |
| 2067 (Chothia) | | |
| SEQ ID NO: | HCDR3 | RKVGKGVYYALDY |
| 2066 (Chothia) | | |
| SEQ ID NO: | HCDR1 | GYTFTNYW |
| 2068 (IMGT) | | |
| SEQ ID NO: | HCDR2 | ITPTTGYP |
| 2069 (IMGT) | | |
| SEQ ID NO: | HCDR3 | ARRKVGKGVYYALDY |
| 2070 (IMGT) | | |
| SEQ ID NO: | HCDR1 | GYTFTNYWMH |
| 2071 (Combined | | |
| Chothia and | | |
| Kabat) | | |
| SEQ ID NO: | HCDR2 | FITPTTGYPEYNQKFKD |
| 2065 (Combined | | |
| Chothia and | | |
| Kabat) | | |
| SEQ ID NO: | HCDR3 | RKVGKGVYYALDY |
| 2066 (Combined | | |
| Chothia and | | |
| Kabat) | | |
| SEQ ID NO: | VH | QVQLVQSGAEVKKPGSSVKVSCKASGYTFTNYWMHW |
| 2087 | | VRQAPGQGLEWMGFITPTTGYPEYNQKFKDRVTITADK |
| | | STSTAYMELSSLRSEDTAVYYCARRKVGKGVYYALDY |
| | | WGQGTTVTVSS |
| SEQ ID NO: | DNA VH | CAAGTCCAACTCGTCCAATCAGGAGCAGAAGTCAAG |
| 2088 | | AAGCCCGGAAGCTCTGTCAAAGTGTCCTGCAAGGCCT |
| | | CCGGTTACACCTTCACCAACTATTGGATGCACTGGGT |
| | | CAGACAGGCCCCGGGACAGGGCTTGGAATGGATGGG |
| | | TTTCATCACTCCAACCACCGGTTACCCGGAGTACAAC |
| | | CAGAAGTTTAAGGACCGCGTGACCATTACTGCCGAC |
| | | AAGTCCACGAGCACCGCTTACATGGAACTTAGCAGC |
| | | CTGCGGTCCGAGGACACTGCCGTGTATTACTGCGCGC |
| | | GGAGGAAGGTCGGAAAGGGAGTGTACTACGCACTGG |
| | | ACTACTGGGGCCAGGGAACCACCGTGACTGTGTCCTC |
| | | C |
| SEQ ID NO: | LCDR1 | RASGNIHNYLA |
| 2074 (Kabat) | | |
| SEQ ID NO: | LCDR2 | NTKTLAD |
| 2075 (Kabat) | | |
| SEQ ID NO: | LCDR3 | QHFWSSPWT |
| 2076 (Kabat) | | |
| SEQ ID NO: | LCDR1 | SGNIHNY |
| 2077 (Chothia) | | |
| SEQ ID NO: | LCDR2 | NTK |
| 2078 (Chothia) | | |
| SEQ ID NO: | LCDR3 | FWSSPW |
| 2079 (Chothia) | | |
| SEQ ID NO: | LCDR1 | GNIHNY |
| 2080 (IMGT) | | |
| SEQ ID NO: | LCDR 2 | NTK |
| 2078 (IMGT) | | |
| SEQ ID NO: | LCDR3 | QHFWSSPWT |
| 2076 (IMGT) | | |
| SEQ ID NO: | LCDR1 | RASGNIHNYLA |
| 2074 (Combined | | |
| Chothia and | | |
| Kabat) | | |
| SEQ ID NO: | LCDR2 | NTKTLAD |
| 2075(Combined | | |
| Chothia and | | |
| Kabat) | | |
| SEQ ID NO: | LCDR3 | QHFWSSPWT |
| 2076 (Combined | | |
| Chothia and | | |
| Kabat) | | |
| SEQ ID NO: | VL | DIQMTQSPSSLSASVGDRVTITCRASGNIHNYLAWYQQ |
| 2081 | | KPGKVPKLLIYNTKTLADGVPSRFSGSGSGTDYTLTIS |
| | | SLQPEDVATYYCQHFWSSPWTFGGGTKVEIK |
| SEQ ID NO: | DNA VL | GATATTCAGATGACCCAGTCCCCTTCATCCCTGAGCG |
| 2089 | | CCTCAGTGGGCGATAGAGTGACCATCACTTGTCGCGC |
| | | CTCGGGCAATATCCACAACTACCTCGCCTGGTACCAG |
| | | CAGAAGCCGGGAAAAGTGCCTAAGCTGCTGATCTAC |
| | | AACACTAAGACCCTGGCGGATGGAGTGCCCAGCCGG |
| | | TTCTCCGGCTCCGGCAGCGGCACAGACTACACCCTCA |
| | | CCATCTCCTCGCTGCAACCAGAGGACGTGGCTACCTA |
| | | CTACTGCCAGCATTTCTGGTCGTCCCCCTGGACTTTCG |
| | | GAGGGGGGACCAAAGTGGAGATTAAG |
| SEQ ID NO: | Linker | GGGGSGGGGSGGGGSGGGGS |
| 1010 | | |
| SEQ ID NO: | scFv (VH- | QVQLVQSGAEVKKPGSSVKVSCKASGYTFTNYWMHW |
| 2090 | linker-VL) | VRQAPGQGLEWMGFITPTTGYPEYNQKFKDRVTITADK |
| | | STSTAYMELSSLRSEDTAVYYCARRKVGKGVYYALDY |
| | | WGQGTTVTVSSGGGGSGGGGSGGGGSGGGGSDIQMTQ |
| | | SPSSLSASVGDRVTITCRASGNIHNYLAWYQQKPGKVP |
| | | KLLIYNTKTLADGVPSRFSGSGSGTDYTLTISSLQPED |
| | | VATYYCQHFWSSPWTFGGGTKVEIK |
| SEQ ID NO: | DNA scFv | CAAGTCCAACTCGTCCAATCAGGAGCAGAAGTCAAG |
| 2091 | (VH-linker- | AAGCCCGGAAGCTCTGTCAAAGTGTCCTGCAAGGCCT |
| | VL) | CCGGTTACACCTTCACCAACTATTGGATGCACTGGGT |
| | | CAGACAGGCCCCGGGACAGGGCTTGGAATGGATGGG |
| | | TTTCATCACTCCAACCACCGGTTACCCGGAGTACAAC |
| | | CAGAAGTTTAAGGACCGCGTGACCATTACTGCCGAC |
| | | AAGTCCACGAGCACCGCTTACATGGAACTTAGCAGC |
| | | CTGCGGTCCGAGGACACTGCCGTGTATTACTGCGCGC |
| | | GGAGGAAGGTCGGAAAGGGAGTGTACTACGCACTGG |
| | | ACTACTGGGGCCAGGGAACCACCGTGACTGTGTCCTC |
| | | CGGTGGCGGAGGGTCGGGAGGGGGGGGCTCGGGAG |
| | | GAGGAGGGTCCGGGGGCGGTGGCTCAGATATTCAGA |
| | | TGACCCAGTCCCCTTCATCCCTGAGCGCCTCAGTGGG |
| | | CGATAGAGTGACCATCACTTGTCGCGCCTCGGGCAAT |
| | | ATCCACAACTACCTCGCCTGGTACCAGCAGAAGCCG |
| | | GGAAAAGTGCCTAAGCTGCTGATCTACAACACTAAG |
| | | ACCCTGGCGGATGGAGTGCCCAGCCGGTTCTCCGGCT |
| | | CCGGCAGCGGCACAGACTACACCCTCACCATCTCCTC |
| | | GCTGCAACCAGAGGACGTGGCTACCTACTACTGCCA |
| | | GCATTTCTGGTCGTCCCCCTGGACTTTCGGAGGGGGG |
| | | ACCAAAGTGGAGATTAAG |
| SEQ ID NO: | Full CAR | MALPVTALLLPLALLLHAARPQVQLVQSGAEVKKPGSS |
| 2092 | amino acid | VKVSCKASGYTFTNYWMHWVRQAPGQGLEWMGFITP |
| | sequence | TTGYPEYNQKFKDRVTITADKSTSTAYMELSSLRSEDTA |
| | | VYYCARRKVGKGVYYALDYWGQGTTVTVSSGGGGSG |
| | | GGGSGGGGSGGGGSDIQMTQSPSSLSASVGDRVTITCR |
| | | ASGNIHNYLAWYQQKPGKVPKLLIYNTKTLADGVPSRF |
| | | SGSGSGTDYTLTISSLQPEDVATYYCQHFWSSPWTFGG |
| | | GTKVEIKTTTPAPRPPTPAPTIASQPLSLRPEACRPAAGG |
| | | AVHTRGLDFACDIYIWAPLAGTCGVLLLSLVITLYCKRG |
| | | RKKLLYIFKQPFMRPVQTTQEEDGCSCRFPEEEEGGCEL |
| | | RVKFSRSADAPAYQQGQNQLYNELNLGRREEYDVLDK |
| | | RRGRDPEMGGKPRRKNPQEGLYNELQKDKMAEAYSEI |
| | | GMKGERRRGKGHDGLYQGLSTATKDTYDALHMQALP |
| | | PR |
| SEQ ID NO: | Full CAR | ATGGCCCTCCCTGTCACCGCCCTGCTGCTTCCGCTGG |
| 2093 | nucleic acid | CTCTTCTGCTCCACGCCGCTCGGCCCCAAGTCCAACT |
| | sequence | CGTCCAATCAGGAGCAGAAGTCAAGAAGCCCGGAAG |
| | | CTCTGTCAAAGTGTCCTGCAAGGCCTCCGGTTACACC |
| | | TTCACCAACTATTGGATGCACTGGGTCAGACAGGCCC |
| | | CGGGACAGGGCTTGGAATGGATGGGTTTCATCACTCC |
| | | AACCACCGGTTACCCGGAGTACAACCAGAAGTTTAA |
| | | GGACCGCGTGACCATTACTGCCGACAAGTCCACGAG |
| | | CACCGCTTACATGGAACTTAGCAGCCTGCGGTCCGAG |
| | | GACACTGCCGTGTATTACTGCGCGCGGAGGAAGGTC |
| | | GGAAAGGGAGTGTACTACGCACTGGACTACTGGGGC |
| | | CAGGGAACCACCGTGACTGTGTCCTCCGGTGGCGGA |
| | | GGGTCGGGAGGGGGGGGCTCGGGAGGAGGAGGGTC |
| | | CGGGGGCGGTGGCTCAGATATTCAGATGACCCAGTC |
| | | CCCTTCATCCCTGAGCGCCTCAGTGGGCGATAGAGTG |
| | | ACCATCACTTGTCGCGCCTCGGGCAATATCCACAACT |
| | | ACCTCGCCTGGTACCAGCAGAAGCCGGGAAAAGTGC |
| | | CTAAGCTGCTGATCTACAACACTAAGACCCTGGCGGA |
| | | TGGAGTGCCCAGCCGGTTCTCCGGCTCCGGCAGCGGC |
| | | ACAGACTACACCCTCACCATCTCCTCGCTGCAACCAG |
| | | AGGACGTGGCTACCTACTACTGCCAGCATTTCTGGTC |
| | | GTCCCCCTGGACTTTCGGAGGGGGGACCAAAGTGGA |
| | | GATTAAGACCACTACCCCAGCACCGAGGCCACCCAC |
| | | CCCGGCTCCTACCATCGCCTCCCAGCCTCTGTCCCTG |
| | | CGTCCGGAGGCATGTAGACCCGCAGCTGGTGGGGCC |
| | | GTGCATACCCGGGGTCTTGACTTCGCCTGCGATATCT |
| | | ACATTTGGGCCCCTCTGGCTGGTACTTGCGGGGTCCT |
| | | GCTGCTTTCACTCGTGATCACTCTTTACTGTAAGCGC |
| | | GGTCGGAAGAAGCTGCTGTACATCTTTAAGCAACCCT |
| | | TCATGAGGCCTGTGCAGACTACTCAAGAGGAGGACG |
| | | GCTGTTCATGCCGGTTCCCAGAGGAGGAGGAAGGCG |
| | | GCTGCGAACTGCGCGTGAAATTCAGCCGCAGCGCAG |
| | | ATGCTCCAGCCTACCAGCAGGGGCAGAACCAGCTCT |
| | | ACAACGAACTCAATCTTGGTCGGAGAGAGGAGTACG |
| | | ACGTGCTGGACAAGCGGAGAGGACGGGACCCAGAAA |
| | | TGGGCGGGAAGCCGCGCAGAAAGAATCCCCAAGAGG |
| | | GCCTGTACAACGAGCTCCAAAAGGATAAGATGGCAG |
| | | AAGCCTATAGCGAGATTGGTATGAAAGGGGAACGCA |
| | | GAAGAGGCAAAGGCCACGACGGACTGTACCAGGGAC |
| | | TCAGCACCGCCACCAAGGACACCTATGACGCTCTTCA |
| | | CATGCAGGCCCTGCCGCCTCGG |
| CD20-C2H3 | | |
| SEQ ID NO: | HCDR1 | NYWMH |
| 2064 (Kabat) | | |
| SEQ ID NO: | HCDR2 | FITPTTGYPEYNQKFKD |
| 2065 (Kabat) | | |
| SEQ ID NO: | HCDR3 | RKVGKGVYYALDY |
| 2066 (Kabat) | | |
| SEQ ID NO: | HCDR1 | GYTFTNY |
| 2022 (Chothia) | | |
| SEQ ID NO: | HCDR2 | TPTTGY |
| 2067 (Chothia) | | |
| SEQ ID NO: | HCDR3 | RKVGKGVYYALDY |
| 2066 (Chothia) | | |
| SEQ ID NO: | HCDR1 | GYTFTNYW |
| 2068 (IMGT) | | |
| SEQ ID NO: | HCDR2 | ITPTTGYP |
| 2069 (IMGT) | | |
| SEQ ID NO: | HCDR3 | ARRKVGKGVYYALDY |
| 2070 (IMGT) | | |
| SEQ ID NO: | HCDR1 | GYTFTNYWMH |
| 2071 (Combined | | |
| Chothia and | | |
| Kabat | | |
| SEQ ID NO: | HCDR2 | FITPTTGYPEYNQKFKD |
| 2065 (Combined | | |
| Chothia and | | |
| Kabat) | | |
| SEQ ID NO: | HCDR3 | RKVGKGVYYALDY |
| 2066 (Combined | | |
| Chothia and | | |
| Kabat) | | |
| SEQ ID NO: | VH | QVQLVQSGAEVKKPGASVKVSCKASGYTFTNYWMHW |
| 2072 | | VRQAPGQGLEWMGFITPTTGYPEYNQKFKDRVTMTAD |
| | | KSTSTAYMELSSLRSEDTAVYYCARRKVGKGVYYALD |
| | | YWGQGTTVTVSS |
| SEQ ID NO: | DNA VH | CAAGTCCAACTCGTCCAGTCCGGTGCAGAAGTCAAG |
| 2094 | | AAACCCGGAGCTTCCGTGAAAGTGTCCTGCAAAGCCT |
| | | CCGGTTACACCTTTACGAACTACTGGATGCATTGGGT |
| | | GCGCCAGGCCCCGGGACAGGGGCTGGAATGGATGGG |
| | | CTTCATTACCCCCACCACCGGATACCCCGAGTACAAT |
| | | CAGAAGTTCAAGGACCGGGTCACCATGACCGCCGAC |
| | | AAGTCAACCTCTACTGCTTACATGGAGCTGTCCAGCC |
| | | TGCGGTCGGAAGATACCGCCGTGTATTACTGCGCGAG |
| | | AAGGAAAGTCGGAAAGGGAGTGTACTATGCCCTGGA |
| | | CTACTGGGGACAGGGGACCACTGTGACTGTGTCAAG |
| | | C |
| SEQ ID NO: | LCDR1 | RASGNIHNYLA |
| 2074 (Kabat) | | |
| SEQ ID NO: | LCDR2 | NTKTLAD |
| 2075 (Kabat) | | |
| SEQ ID NO: | LCDR3 | QHFWSSPWT |
| 2076 (Kabat) | | |
| SEQ ID NO: | LCDR1 | SGNIHNY |
| 2077 (Chothia) | | |
| SEQ ID NO: | LCDR2 | NTK |
| 2078 (Chothia) | | |
| SEQ ID NO: | LCDR3 | FWSSPW |
| 2079 (Chothia) | | |
| SEQ ID NO: | LCDR1 | GNIHNY |
| 2080 (IMGT) | | |
| SEQ ID NO: | LCDR2 | NTK |
| 2078 (IMGT) | | |
| SEQ ID NO: | LCDR3 | QHFWSSPWT |
| 2076 (IMGT) | | |
| SEQ ID NO: | LCDR1 | RASGNIHNYLA |
| 2074 (Combined | | |
| Chothia and | | |
| Kabat) | | |
| SEQ ID NO: | LCDR2 | NTKTLAD |
| 2075 (Combined | | |
| Chothia and | | |
| Kabat) | | |
| SEQ ID NO: | LCDR3 | QHFWSSPWT |
| 2076 (Combined | | |
| Chothia and | | |
| Kabat) | | |
| SEQ ID NO: | VL | AIRMTQSPFSLSASVGDRVTITCRASGNIHNYLAWYQQ |
| 2095 | | KPAKAPKLFIYNTKTLADGVPSRFSGSGSGTDYTLTIS |
| | | SLQPEDFATYYCQHFWSSPWTFGGGTKVEIK |
| SEQ ID NO: | DNA VL | GCGATCCGCATGACCCAGAGCCCGTTCTCCCTGTCCG |
| 2096 | | CGTCCGTGGGGGACCGCGTGACTATCACGTGTCGGGC |
| | | CTCCGGGAACATCCACAACTACCTCGCATGGTACCAG |
| | | CAGAAGCCGGCCAAGGCCCCTAAGTTGTTCATCTACA |
| | | ACACCAAGACTCTTGCCGACGGAGTGCCGTCCCGGTT |
| | | TAGCGGAAGCGGTTCCGGCACCGACTACACCCTGACT |
| | | ATCTCGAGCCTGCAACCAGAAGATTTCGCCACTTACT |
| | | ACTGCCAGCACTTCTGGTCGTCCCCTTGGACATTCGG |
| | | CGGCGGCACCAAGGTCGAGATTAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTNYWMHW |
| 2097 | linker-VL) | VRQAPGQGLEWMGFITPTTGYPEYNQKFKDRVTMTAD |
| | | KSTSTAYMELSSLRSEDTAVYYCARRKVGKGVYYALD |
| | | YWGQGTTVTVSSGGGGSGGGGSGGGGSGGGGSAIRMT |
| | | QSPFSLSASVGDRVTITCRASGNIHNYLAWYQQKPAKA |
| | | PKLFIYNTKTLADGVPSRFSGSGSGTDYTLTISSLQPE |
| | | DFATYYCQHFWSSPWTFGGGTKVEIK |
| SEQ ID NO: | DNA scFv | CAAGTCCAACTCGTCCAGTCCGGTGCAGAAGTCAAG |
| 2098 | (VH-linker- | AAACCCGGAGCTTCCGTGAAAGTGTCCTGCAAAGCCT |
| | VL) | CCGGTTACACCTTTACGAACTACTGGATGCATTGGGT |
| | | GCGCCAGGCCCCGGGACAGGGGCTGGAATGGATGGG |
| | | CTTCATTACCCCCACCACCGGATACCCCGAGTACAAT |
| | | CAGAAGTTCAAGGACCGGGTCACCATGACCGCCGAC |
| | | AAGTCAACCTCTACTGCTTACATGGAGCTGTCCAGCC |
| | | TGCGGTCGGAAGATACCGCCGTGTATTACTGCGCGAG |
| | | AAGGAAAGTCGGAAAGGGAGTGTACTATGCCCTGGA |
| | | CTACTGGGGACAGGGGACCACTGTGACTGTGTCAAG |
| | | CGGAGGCGGAGGCTCGGGGGGCGGAGGTTCGGGCGG |
| | | AGGAGGATCAGGGGGCGGCGGTTCCGCGATCCGCAT |
| | | GACCCAGAGCCCGTTCTCCCTGTCCGCGTCCGTGGGG |
| | | GACCGCGTGACTATCACGTGTCGGGCCTCCGGGAAC |
| | | ATCCACAACTACCTCGCATGGTACCAGCAGAAGCCG |
| | | GCCAAGGCCCCTAAGTTGTTCATCTACAACACCAAGA |
| | | CTCTTGCCGACGGAGTGCCGTCCCGGTTTAGCGGAAG |
| | | CGGTTCCGGCACCGACTACACCCTGACTATCTCGAGC |
| | | CTGCAACCAGAAGATTTCGCCACTTACTACTGCCAGC |
| | | ACTTCTGGTCGTCCCCTTGGACATTCGGCGGCGGCAC |
| | | CAAGGTCGAGATTAAG |
| SEQ ID NO: | Full CAR | MALPVTALLLPLALLLHAARPQVQLVQSGAEVKKPGAS |
| 2099 | amino acid | VKVSCKASGYTFTNYWMHWVRQAPGQGLEWMGFITP |
| | sequence | TTGYPEYNQKFKDRVTMTADKSTSTAYMELSSLRSEDT |
| | | AVYYCARRKVGKGVYYALDYWGQGTTVTVSSGGGGS |
| | | GGGGSGGGGSGGGGSAIRMTQSPFSLSASVGDRVTITC |
| | | RASGNIHNYLAWYQQKPAKAPKLFIYNTKTLADGVPSR |
| | | FSGSGSGTDYTLTISSLQPEDFATYYCQHFWSSPWTFGG |
| | | GTKVEIKTTTPAPRPPTPAPTIASQPLSLRPEACRPAAGG |
| | | AVHTRGLDFACDIYIWAPLAGTCGVLLLSLVITLYCKRG |
| | | RKKLLYIFKQPFMRPVQTTQEEDGCSCRFPEEEEGGCEL |
| | | RVKFSRSADAPAYQQGQNQLYNELNLGRREEYDVLDK |
| | | RRGRDPEMGGKPRRKNPQEGLYNELQKDKMAEAYSEI |
| | | GMKGERRRGKGHDGLYQGLSTATKDTYDALHMQALP |
| | | PR |
| SEQ ID NO: | Full CAR | ATGGCCCTCCCTGTCACCGCCCTGCTGCTTCCGCTGG |
| 2100 | nucleic acid | CTCTTCTGCTCCACGCCGCTCGGCCCCAAGTCCAACT |
| | sequence | CGTCCAGTCCGGTGCAGAAGTCAAGAAACCCGGAGC |
| | | TTCCGTGAAAGTGTCCTGCAAAGCCTCCGGTTACACC |
| | | TTTACGAACTACTGGATGCATTGGGTGCGCCAGGCCC |
| | | CGGGACAGGGGCTGGAATGGATGGGCTTCATTACCC |
| | | CCACCACCGGATACCCCGAGTACAATCAGAAGTTCA |
| | | AGGACCGGGTCACCATGACCGCCGACAAGTCAACCT |
| | | CTACTGCTTACATGGAGCTGTCCAGCCTGCGGTCGGA |
| | | AGATACCGCCGTGTATTACTGCGCGAGAAGGAAAGT |
| | | CGGAAAGGGAGTGTACTATGCCCTGGACTACTGGGG |
| | | ACAGGGGACCACTGTGACTGTGTCAAGCGGAGGCGG |
| | | AGGCTCGGGGGGCGGAGGTTCGGGCGGAGGAGGATC |
| | | AGGGGGCGGCGGTTCCGCGATCCGCATGACCCAGAG |
| | | CCCGTTCTCCCTGTCCGCGTCCGTGGGGGACCGCGTG |
| | | ACTATCACGTGTCGGGCCTCCGGGAACATCCACAACT |
| | | ACCTCGCATGGTACCAGCAGAAGCCGGCCAAGGCCC |
| | | CTAAGTIGTTCATCTACAACACCAAGACTCTTGCCGA |
| | | CGGAGTGCCGTCCCGGTTTAGCGGAAGCGGTTCCGGC |
| | | ACCGACTACACCCTGACTATCTCGAGCCTGCAACCAG |
| | | AAGATTTCGCCACTTACTACTGCCAGCACTTCTGGTC |
| | | GTCCCCTTGGACATTCGGCGGCGGCACCAAGGTCGA |
| | | GATTAAGACCACTACCCCAGCACCGAGGCCACCCAC |
| | | CCCGGCTCCTACCATCGCCTCCCAGCCTCTGTCCCTG |
| | | CGTCCGGAGGCATGTAGACCCGCAGCTGGTGGGGCC |
| | | GTGCATACCCGGGGTCTTGACTTCGCCTGCGATATCT |
| | | ACATTTGGGCCCCTCTGGCTGGTACTTGCGGGGTCCT |
| | | GCTGCTTTCACTCGTGATCACTCTTTACTGTAAGCGC |
| | | GGTCGGAAGAAGCTGCTGTACATCTTTAAGCAACCCT |
| | | TCATGAGGCCTGTGCAGACTACTCAAGAGGAGGACG |
| | | GCTGTTCATGCCGGTTCCCAGAGGAGGAGGAAGGCG |
| | | GCTGCGAACTGCGCGTGAAATTCAGCCGCAGCGCAG |
| | | ATGCTCCAGCCTACCAGCAGGGGCAGAACCAGCTCT |
| | | ACAACGAACTCAATCTTGGTCGGAGAGAGGAGTACG |
| | | ACGTGCTGGACAAGCGGAGAGGACGGGACCCAGAAA |
| | | TGGGCGGGAAGCCGCGCAGAAAGAATCCCCAAGAGG |
| | | GCCTGTACAACGAGCTCCAAAAGGATAAGATGGCAG |
| | | AAGCCTATAGCGAGATTGGTATGAAAGGGGAACGCA |
| | | GAAGAGGCAAAGGCCACGACGGACTGTACCAGGGAC |
| | | TCAGCACCGCCACCAAGGACACCTATGACGCTCTTCA |
| | | CATGCAGGCCCTGCCGCCTCGG |
| CD20-C2H4 | | |
| SEQ ID NO: | HCDR1 | NYWMH |
| 2064 (Kabat) | | |
| SEQ ID NO: | HCDR2 | FITPTTGYPEYNQKFKD |
| 2065 (Kabat) | | |
| SEQ ID NO: | HCDR3 | RKVGKGVYYALDY |
| 2066 (Kabat) | | |
| SEQ ID NO: | HCDR1 | GYTFTNY |
| 2022 (Chothia) | | |
| SEQ ID NO: | HCDR2 | TPTTGY |
| 2067 (Chothia) | | |
| SEQ ID NO: | HCDR3 | RKVGKGVYYALDY |
| 2066 (Chothia) | | |
| SEQ ID NO: | HCDR1 | GYTFTNYW |
| 2068 (IMGT) | | |
| SEQ ID NO: | HCDR2 | ITPTTGYP |
| 2069 (IMGT) | | |
| SEQ ID NO: | HCDR3 | ARRKVGKGVYYALDY |
| 2070 (IMGT) | | |
| SEQ ID NO: | HCDR1 | GYTFTNYWMH |
| 2071 (Combined | | |
| Chothia and | | |
| Kabat) | | |
| SEQ ID NO: | HCDR2 | FITPTTGYPEYNQKFKD |
| 2065 (Combined | | |
| Chothia and | | |
| Kabat) | | |
| SEQ ID NO: | HCDR3 | RKVGKGVYYALDY |
| 2066 (Combined | | |
| Chothia and | | |
| Kabat) | | |
| SEQ ID NO: | VH | QVQLVQSGAEVKKPGSSVKVSCKASGYTFTNYWMHW |
| 2087 | | VRQAPGQGLEWMGFITPTTGYPEYNQKFKDRVTITADK |
| | | STSTAYMELSSLRSEDTAVYYCARRKVGKGVYYALDY |
| | | WGQGTTVTVSS |
| SEQ ID NO: | DNA VH | CAAGTCCAACTCGTCCAAAGCGGTGCAGAAGTCAAG |
| 2101 | | AAGCCCGGTTCCTCCGTGAAAGTGTCCTGCAAAGCCT |
| | | CGGGCTACACCTTCACTAATTACTGGATGCATTGGGT |
| | | CCGCCAGGCGCCCGGACAGGGATTGGAATGGATGGG |
| | | GTTCATCACGCCGACCACCGGATACCCGGAGTACAA |
| | | CCAGAAGTTCAAGGACAGAGTGACCATTACCGCCGA |
| | | TAAGTCCACCTCCACCGCTTACATGGAGCTCTCCTCA |
| | | CTGCGGTCCGAAGATACAGCCGTGTACTATTGTGCTC |
| | | GCCGGAAAGTCGGAAAGGGAGTGTACTACGCCCTGG |
| | | ACTATTGGGGCCAGGGCACCACCGTGACCGTGTCCTC |
| | | G |
| SEQ ID NO: | LCDR1 | RASGNIHNYLA |
| 2074 (Kabat) | | |
| SEQ ID NO: | LCDR2 | NTKTLAD |
| 2075 (Kabat) | | |
| SEQ ID NO: | LCDR3 | QHFWSSPWT |
| 2076 (Kabat) | | |
| SEQ ID NO: | LCDR1 | SGNIHNY |
| 2077 (Chothia) | | |
| SEQ ID NO: | LCDR2 | NTK |
| 2078 (Chothia) | | |
| SEQ ID NO: | LCDR3 | FWSSPW |
| 2079 (Chothia) | | |
| SEQ ID NO: | LCDR1 | GNIHNY |
| 2080 (IMGT) | | |
| SEQ ID NO: | LCDR2 | NTK |
| 2078 (IMGT) | | |
| SEQ ID NO: | LCDR3 | QHFWSSPWT |
| 2076 (IMGT) | | |
| SEQ ID NO: | LCDR1 | RASGNIHNYLA |
| 2074 (Combined | | |
| Chothia and | | |
| Kabat) | | |
| SEQ ID NO: | LCDR2 | NTKTLAD |
| 2075 (Combined | | |
| Chothia and | | |
| Kabat) | | |
| SEQ ID NO: | LCDR3 | QHFWSSPWT |
| 2076 (Combined | | |
| Chothia and | | |
| Kabat) | | |
| SEQ ID NO: | VL | AIRMTQSPFSLSASVGDRVTITCRASGNIHNYLAWYQQ |
| 2095 | | KPAKAPKLFIYNTKTLADGVPSRFSGSGSGTDYTLTISSL |
| | | QPEDFATYYCQHFWSSPWTFGGGTKVEIK |
| SEQ ID NO: | DNA VL | GCCATTAGGATGACTCAGTCCCCTTTCTCCCTCTCCGC |
| 2102 | | GAGCGTGGGCGACCGCGTGACGATCACTTGCCGGGC |
| | | CTCGGGGAACATTCACAACTACCTGGCCTGGTACCAG |
| | | CAGAAGCCGGCCAAGGCCCCTAAGCTGTTCATCTACA |
| | | ACACCAAGACCCTTGCGGACGGAGTGCCATCGAGAT |
| | | TTTCCGGCTCGGGCTCTGGGACCGATTACACTCTGAC |
| | | TATCTCAAGCCTGCAACCTGAGGACTTCGCCACTTAC |
| | | TACTGCCAGCACTTCTGGAGCAGCCCCTGGACTTTCG |
| | | GTGGCGGGACCAAGGTCGAAATCAAG |
| SEQ ID NO: | Linker | GGGGSGGGGSGGGGSGGGGS |
| 1010 | | |
| SEQ ID NO: | scFv (VH- | QVQLVQSGAEVKKPGSSVKVSCKASGYTFTNYWMHW |
| 2103 | linker-VL) | VRQAPGQGLEWMGFITPTTGYPEYNQKFKDRVTITADK |
| | | STSTAYMELSSLRSEDTAVYYCARRKVGKGVYYALDY |
| | | WGQGTTVTVSSGGGGSGGGGSGGGGSGGGGSAIRMTQ |
| | | SPFSLSASVGDRVTITCRASGNIHNYLAWYQQKPAKAP |
| | | KLFIYNTKTLADGVPSRFSGSGSGTDYTLTISSLQPEDFA |
| | | TYYCQHFWSSPWTFGGGTKVEIK |
| SEQ ID NO: | DNA scFv | CAAGTCCAACTCGTCCAAAGCGGTGCAGAAGTCAAG |
| 2104 | (VH-linker- | AAGCCCGGTTCCTCCGTGAAAGTGTCCTGCAAAGCCT |
| | VL) | CGGGCTACACCTTCACTAATTACTGGATGCATTGGGT |
| | | CCGCCAGGCGCCCGGACAGGGATTGGAATGGATGGG |
| | | GTTCATCACGCCGACCACCGGATACCCGGAGTACAA |
| | | CCAGAAGTTCAAGGACAGAGTGACCATTACCGCCGA |
| | | TAAGTCCACCTCCACCGCTTACATGGAGCTCTCCTCA |
| | | CTGCGGTCCGAAGATACAGCCGTGTACTATTGTGCTC |
| | | GCCGGAAAGTCGGAAAGGGAGTGTACTACGCCCTGG |
| | | ACTATTGGGGCCAGGGCACCACCGTGACCGTGTCCTC |
| | | GGGAGGAGGGGGTTCGGGCGGAGGCGGCTCCGGTGG |
| | | AGGCGGAAGCGGAGGGGGCGGATCAGCCATTAGGAT |
| | | GACTCAGTCCCCTTTCTCCCTCTCCGCGAGCGTGGGC |
| | | GACCGCGTGACGATCACTTGCCGGGCCTCGGGGAAC |
| | | ATTCACAACTACCTGGCCTGGTACCAGCAGAAGCCG |
| | | GCCAAGGCCCCTAAGCTGTTCATCTACAACACCAAGA |
| | | CCCTTGCGGACGGAGTGCCATCGAGATTTTCCGGCTC |
| | | GGGCTCTGGGACCGATTACACTCTGACTATCTCAAGC |
| | | CTGCAACCTGAGGACTTCGCCACTTACTACTGCCAGC |
| | | ACTTCTGGAGCAGCCCCTGGACTTTCGGTGGCGGGAC |
| | | CAAGGTCGAAATCAAG |
| SEQ ID NO: | Full CAR | MALPVTALLLPLALLLHAARPQVQLVQSGAEVKKPGSS |
| 2105 | amino acid | VKVSCKASGYTFTNYWMHWVRQAPGQGLEWMGFITP |
| | sequence | TTGYPEYNQKFKDRVTITADKSTSTAYMELSSLRSEDTA |
| | | VYYCARRKVGKGVYYALDYWGQGTTVTVSSGGGGSG |
| | | GGGSGGGGSGGGGSAIRMTQSPFSLSASVGDRVTITCR |
| | | ASGNIHNYLAWYQQKPAKAPKLFIYNTKTLADGVPSRF |
| | | SGSGSGTDYTLTISSLQPEDFATYYCQHFWSSPWTFGGG |
| | | TKVEIKTTTPAPRPPTPAPTIASQPLSLRPEACRPAAGGA |
| | | VHTRGLDFACDIYIWAPLAGTCGVLLLSLVITLYCKRGR |
| | | KKLLYIFKQPFMRPVQTTQEEDGCSCRFPEEEEGGCELR |
| | | VKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPP |
| | | R |
| SEQ ID NO: | Full CAR | ATGGCCCTCCCTGTCACCGCCCTGCTGCTTCCGCTGG |
| 2106 | nucleic acid | CTCTTCTGCTCCACGCCGCTCGGCCCCAAGTCCAACT |
| | sequence | CGTCCAAAGCGGTGCAGAAGTCAAGAAGCCCGGTTC |
| | | CTCCGTGAAAGTGTCCTGCAAAGCCTCGGGCTACACC |
| | | TTCACTAATTACTGGATGCATTGGGTCCGCCAGGCGC |
| | | CCGGACAGGGATTGGAATGGATGGGGTTCATCACGC |
| | | CGACCACCGGATACCCGGAGTACAACCAGAAGTTCA |
| | | AGGACAGAGTGACCATTACCGCCGATAAGTCCACCT |
| | | CCACCGCTTACATGGAGCTCTCCTCACTGCGGTCCGA |
| | | AGATACAGCCGTGTACTATTGTGCTCGCCGGAAAGTC |
| | | GGAAAGGGAGTGTACTACGCCCTGGACTATTGGGGC |
| | | CAGGGCACCACCGTGACCGTGTCCTCGGGAGGAGGG |
| | | GGTTCGGGCGGAGGCGGCTCCGGTGGAGGCGGAAGC |
| | | GGAGGGGGCGGATCAGCCATTAGGATGACTCAGTCC |
| | | CCTTTCTCCCTCTCCGCGAGCGTGGGCGACCGCGTGA |
| | | CGATCACTTGCCGGGCCTCGGGGAACATTCACAACTA |
| | | CCTGGCCTGGTACCAGCAGAAGCCGGCCAAGGCCCC |
| | | TAAGCTGTTCATCTACAACACCAAGACCCTTGCGGAC |
| | | GGAGTGCCATCGAGATTTTCCGGCTCGGGCTCTGGGA |
| | | CCGATTACACTCTGACTATCTCAAGCCTGCAACCTGA |
| | | GGACTTCGCCACTTACTACTGCCAGCACTTCTGGAGC |
| | | AGCCCCTGGACTTTCGGTGGCGGGACCAAGGTCGAA |
| | | ATCAAGACCACTACCCCAGCACCGAGGCCACCCACC |
| | | CCGGCTCCTACCATCGCCTCCCAGCCTCTGTCCCTGC |
| | | GTCCGGAGGCATGTAGACCCGCAGCTGGTGGGGCCG |
| | | TGCATACCCGGGGTCTTGACTTCGCCTGCGATATCTA |
| | | CATTTGGGCCCCTCTGGCTGGTACTTGCGGGGTCCTG |
| | | CTGCTTTCACTCGTGATCACTCTTTACTGTAAGCGCG |
| | | GTCGGAAGAAGCTGCTGTACATCTTTAAGCAACCCTT |
| | | CATGAGGCCTGTGCAGACTACTCAAGAGGAGGACGG |
| | | CTGTTCATGCCGGTTCCCAGAGGAGGAGGAAGGCGG |
| | | CTGCGAACTGCGCGTGAAATTCAGCCGCAGCGCAGA |
| | | TGCTCCAGCCTACCAGCAGGGGCAGAACCAGCTCTA |
| | | CAACGAACTCAATCTTGGTCGGAGAGAGGAGTACGA |
| | | CGTGCTGGACAAGCGGAGAGGACGGGACCCAGAAAT |
| | | GGGCGGGAAGCCGCGCAGAAAGAATCCCCAAGAGG |
| | | GCCTGTACAACGAGCTCCAAAAGGATAAGATGGCAG |
| | | AAGCCTATAGCGAGATTGGTATGAAAGGGGAACGCA |
| | | GAAGAGGCAAAGGCCACGACGGACTGTACCAGGGAC |
| | | TCAGCACCGCCACCAAGGACACCTATGACGCTCTTCA |
| | | CATGCAGGCCCTGCCGCCTCGG |
| CD20-C3H1 | | |
| SEQ ID NO: | HCDR1 | NYNLH |
| 2019 (Kabat) | | |
| SEQ ID NO: | HCDR2 | AIYPGNYDTSYNQKFKG |
| 2020 (Kabat) | | |
| SEQ ID NO: | HCDR3 | VDFGHSRYWYFDV |
| 2021 (Kabat) | | |
| SEQ ID NO: | HCDR1 | GYTFTNY |
| 2022 (Chothia) | | |
| SEQ ID NO: | HCDR2 | YPGNYD |
| 2023 (Chothia) | | |
| SEQ ID NO: | HCDR3 | VDFGHSRYWYFDV |
| 2021 (Chothia) | | |
| SEQ ID NO: | HCDR1 | GYTFTNYN |
| 2024 (IMGT) | | |
| SEQ ID NO: | HCDR2 | IYPGNYDT |
| 2025 (IMGT) | | |
| SEQ ID NO: | HCDR3 | ARVDFGHSRYWYFDV |
| 2026 (IMGT) | | |
| SEQ ID NO: | HCDR1 | GYTFTNYNLH |
| 2027 (Combined | | |
| Chothia and | | |
| Kabat) | | |
| SEQ ID NO: | HCDR2 | AIYPGNYDTSYNQKFKG |
| 2020 (Combined | | |
| Chothia and | | |
| Kabat) | | |
| SEQ ID NO: | HCDR3 | VDFGHSRYWYFDV |
| 2021 (Combined | | |
| Chothia and | | |
| Kabat) | | |
| SEQ ID NO: | VH | QVQLVQSGAEVKKPGASVKVSCKASGYTFTNYNLHWV |
| 2028 | | RQAPGQGLEWMGAIYPGNYDTSYNQKFKGRVTMTAD |
| | | KSTSTAYMELSSLRSEDTAVYYCARVDFGHSRYWYFD |
| | | VWGQGTTVTVSS |
| SEQ ID NO: | DNA VH | CAAGTCCAACTCGTCCAATCCGGTGCAGAAGTCAAG |
| 2107 | | AAACCCGGTGCATCCGTGAAAGTGTCATGCAAAGCC |
| | | TCCGGGTACACCTTCACTAACTACAACCTCCACTGGG |
| | | TCCGCCAGGCCCCGGGACAGGGACTGGAGTGGATGG |
| | | GGGCCATCTACCCGGGAAACTACGACACTTCATACA |
| | | ACCAGAAGTTCAAGGGCAGAGTGACCATGACTGCCG |
| | | ACAAGAGCACATCGACCGCCTACATGGAACTCAGCT |
| | | CCCTGCGCTCCGAGGATACTGCCGTCTACTACTGTGC |
| | | CCGGGTGGACTTCGGCCACTCCCGGTATTGGTATTTC |
| | | GATGTCTGGGGACAGGGAACCACCGTGACTGTGTCC |
| | | AGC |
| SEQ ID NO: | LCDR1 | RATSSVSSMN |
| 2030 (Kabat) | | |
| SEQ ID NO: | LCDR2 | ATSNLAS |
| 2031 (Kabat) | | |
| SEQ ID NO: | LCDR3 | QQWTFNPPT |
| 2032 (Kabat) | | |
| SEQ ID NO: | LCDR1 | TSSVSS |
| 2033 (Chothia) | | |
| SEQ ID NO: | LCDR2 | ATS |
| 2034 (Chothia) | | |
| SEQ ID NO: | LCDR3 | WTFNPP |
| 2035 (Chothia) | | |
| SEQ ID NO: | LCDR1 | SSVSS |
| 2036 (IMGT) | | |
| SEQ ID NO: | LCDR2 | ATS |
| 2034 (IMGT) | | |
| SEQ ID NO: | LCDR3 | QQWTFNPPT |
| 2032 (IMGT) | | |
| SEQ ID NO: | LCDR1 | RATSSVSSMN |
| 2030 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWTFNPPT |
| 2032 (Combined | | |
| Chothia and | | |
| Kabat) | | |
| SEQ ID NO: | VL | EIVLTQSPATLSLSPGERATLSCRATSSVSSMNWYQQKP |
| 2108 | | GQAPRPLIHATSNLASGIPARFSGSGSGTDYTLTISSLEPE |
| | | DAAVYYCQQWTFNPPTFGQGTKLEIK |
| SEQ ID NO: | DNA VL | GAAATCGTGCTGACCCAGTCCCCTGCGACTCTGAGCC |
| 2109 | | TGAGCCCTGGGGAACGCGCCACTTTGTCATGCCGGGC |
| | | CACCTCCTCCGTGTCCTCCATGAACTGGTACCAGCAG |
| | | AAGCCCGGACAGGCTCCGCGGCCGCTGATCCATGCC |
| | | ACCTCCAACCTGGCCAGCGGCATTCCCGCGAGGTTTT |
| | | CCGGCTCGGGCTCTGGTACCGACTACACCCTGACCAT |
| | | CTCGAGCCTTGAGCCAGAAGATGCTGCGGTGTACTAC |
| | | TGCCAACAGTGGACCTTCAATCCGCCTACGTTCGGAC |
| | | AGGGGACCAAGCTGGAGATTAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTNYNLHWV |
| 2110 | linker-VL) | RQAPGQGLEWMGAIYPGNYDTSYNQKFKGRVTMTAD |
| | | KSTSTAYMELSSLRSEDTAVYYCARVDFGHSRYWYFD |
| | | VWGQGTTVTVSSGGGGSGGGGSGGGGSGGGGSEIVLT |
| | | QSPATLSLSPGERATLSCRATSSVSSMNWYQQKPGQAP |
| | | RPLIHATSNLASGIPARFSGSGSGTDYTLTISSLEPE |
| | | DAAVYYCQQWTFNPPTFGQGTKLEIK |
| SEQ ID NO: | DNA scFv | CAAGTCCAACTCGTCCAATCCGGTGCAGAAGTCAAG |
| 2111 | (VH-linker- | AAACCCGGTGCATCCGTGAAAGTGTCATGCAAAGCC |
| | VL) | TCCGGGTACACCTTCACTAACTACAACCTCCACTGGG |
| | | TCCGCCAGGCCCCGGGACAGGGACTGGAGTGGATGG |
| | | GGGCCATCTACCCGGGAAACTACGACACTTCATACA |
| | | ACCAGAAGTTCAAGGGCAGAGTGACCATGACTGCCG |
| | | ACAAGAGCACATCGACCGCCTACATGGAACTCAGCT |
| | | CCCTGCGCTCCGAGGATACTGCCGTCTACTACTGTGC |
| | | CCGGGTGGACTTCGGCCACTCCCGGTATTGGTATTTC |
| | | GATGTCTGGGGACAGGGAACCACCGTGACTGTGTCC |
| | | AGCGGGGGCGGAGGATCGGGTGGCGGAGGTTCGGGG |
| | | GGAGGAGGATCAGGCGGCGGCGGATCGGAAATCGTG |
| | | CTGACCCAGTCCCCTGCGACTCTGAGCCTGAGCCCTG |
| | | GGGAACGCGCCACTTTGTCATGCCGGGCCACCTCCTC |
| | | CGTGTCCTCCATGAACTGGTACCAGCAGAAGCCCGG |
| | | ACAGGCTCCGCGGCCGCTGATCCATGCCACCTCCAAC |
| | | CTGGCCAGCGGCATTCCCGCGAGGTTTTCCGGCTCGG |
| | | GCTCTGGTACCGACTACACCCTGACCATCTCGAGCCT |
| | | TGAGCCAGAAGATGCTGCGGTGTACTACTGCCAACA |
| | | GTGGACCTTCAATCCGCCTACGTTCGGACAGGGGACC |
| | | AAGCTGGAGATTAAG |
| SEQ ID NO: | Full CAR | MALPVTALLLPLALLLHAARPQVQLVQSGAEVKKPGAS |
| 2112 | amino acid | VKVSCKASGYTFTNYNLHWVRQAPGQGLEWMGAIYP |
| | sequence | GNYDTSYNQKFKGRVTMTADKSTSTAYMELSSLRSED |
| | | TAVYYCARVDFGHSRYWYFDVWGQGTTVTVSSGGGG |
| | | SGGGGSGGGGSGGGGSEIVLTQSPATLSLSPGERATLSC |
| | | RATSSVSSMNWYQQKPGQAPRPLIHATSNLASGIPARFS |
| | | GSGSGTDYTLTISSLEPEDAAVYYCQQWTFNPPTFGQGT |
| | | KLEIKTTTPAPRPPTPAPTIASQPLSLRPEACRPAAGGAV |
| | | HTRGLDFACDIYIWAPLAGTCGVLLLSLVITLYCKRGRK |
| | | KLLYIFKQPFMRPVQTTQEEDGCSCRFPEEEEGGCELRV |
| | | KFSRSADAPAYQQGQNQLYNELNLGRREEYDVLDKRR |
| | | GRDPEMGGKPRRKNPQEGLYNELQKDKMAEAYSEIGM |
| | | KGERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2113 | nucleic acid | CTCTTCTGCTCCACGCCGCTCGGCCCCAAGTCCAACT |
| | sequence | CGTCCAATCCGGTGCAGAAGTCAAGAAACCCGGTGC |
| | | ATCCGTGAAAGTGTCATGCAAAGCCTCCGGGTACACC |
| | | TTCACTAACTACAACCTCCACTGGGTCCGCCAGGCCC |
| | | CGGGACAGGGACTGGAGTGGATGGGGGCCATCTACC |
| | | CGGGAAACTACGACACTTCATACAACCAGAAGTTCA |
| | | AGGGCAGAGTGACCATGACTGCCGACAAGAGCACAT |
| | | CGACCGCCTACATGGAACTCAGCTCCCTGCGCTCCGA |
| | | GGATACTGCCGTCTACTACTGTGCCCGGGTGGACTTC |
| | | GGCCACTCCCGGTATTGGTATTTCGATGTCTGGGGAC |
| | | AGGGAACCACCGTGACTGTGTCCAGCGGGGGCGGAG |
| | | GATCGGGTGGCGGAGGTTCGGGGGGAGGAGGATCAG |
| | | GCGGCGGCGGATCGGAAATCGTGCTGACCCAGTCCC |
| | | CTGCGACTCTGAGCCTGAGCCCTGGGGAACGCGCCA |
| | | CTTTGTCATGCCGGGCCACCTCCTCCGTGTCCTCCATG |
| | | AACTGGTACCAGCAGAAGCCCGGACAGGCTCCGCGG |
| | | CCGCTGATCCATGCCACCTCCAACCTGGCCAGCGGCA |
| | | TTCCCGCGAGGTTTTCCGGCTCGGGCTCTGGTACCGA |
| | | CTACACCCTGACCATCTCGAGCCTTGAGCCAGAAGAT |
| | | GCTGCGGTGTACTACTGCCAACAGTGGACCTTCAATC |
| | | CGCCTACGTTCGGACAGGGGACCAAGCTGGAGATTA |
| | | AGACCACTACCCCAGCACCGAGGCCACCCACCCCGG |
| | | CTCCTACCATCGCCTCCCAGCCTCTGTCCCTGCGTCCG |
| | | GAGGCATGTAGACCCGCAGCTGGTGGGGCCGTGCAT |
| | | ACCCGGGGTCTTGACTTCGCCTGCGATATCTACATTT |
| | | GGGCCCCTCTGGCTGGTACTTGCGGGGTCCTGCTGCT |
| | | TTCACTCGTGATCACTCTTTACTGTAAGCGCGGTCGG |
| | | AAGAAGCTGCTGTACATCTTTAAGCAACCCTTCATGA |
| | | GGCCTGTGCAGACTACTCAAGAGGAGGACGGCTGTT |
| | | CATGCCGGTTCCCAGAGGAGGAGGAAGGCGGCTGCG |
| | | AACTGCGCGTGAAATTCAGCCGCAGCGCAGATGCTC |
| | | CAGCCTACCAGCAGGGGCAGAACCAGCTCTACAACG |
| | | AACTCAATCTTGGTCGGAGAGAGGAGTACGACGTGC |
| | | TGGACAAGCGGAGAGGACGGGACCCAGAAATGGGC |
| | | GGGAAGCCGCGCAGAAAGAATCCCCAAGAGGGCCTG |
| | | TACAACGAGCTCCAAAAGGATAAGATGGCAGAAGCC |
| | | TATAGCGAGATTGGTATGAAAGGGGAACGCAGAAGA |
| | | GGCAAAGGCCACGACGGACTGTACCAGGGACTCAGC |
| | | ACCGCCACCAAGGACACCTATGACGCTCTTCACATGC |
| | | AGGCCCTGCCGCCTCGG |
| CD20-C3H3 | | |
| SEQ ID NO: | HCDR1 | NYNLH |
| 2019 (Kabat) | | |
| SEQ ID NO: | HCDR2 | AIYPGNYDTSYNQKFKG |
| 2020 (Kabat) | | |
| SEQ ID NO: | HCDR3 | VDFGHSRYWYFDV |
| 2021 (Kabat) | | |
| SEQ ID NO: | HCDR1 | GYTFTNY |
| 2022 (Chothia) | | |
| SEQ ID NO: | HCDR2 | YPGNYD |
| 2023 (Chothia) | | |
| SEQ ID NO: | HCDR3 | VDFGHSRYWYFDV |
| 2021 (Chothia) | | |
| SEQ ID NO: | HCDR1 | GYTFTNYN |
| 2024 (IMGT) | | |
| SEQ ID NO: | HCDR2 | IYPGNYDT |
| 2025 (IMGT) | | |
| SEQ ID NO: | HCDR3 | ARVDFGHSRYWYFDV |
| 2026 (IMGT) | | |
| SEQ ID NO: | VH | QVQLVQSGAEVKKPGASVKVSCKASGYTFTNYWMHW |
| 2072 | | VRQAPGQGLEWMGFITPTTGYPEYNQKFKDRVTMTAD |
| | | KSTSTAYMELSSLRSEDTAVYYCARRKVGKGVYYALD |
| | | YWGQGTTVTVSS |
| SEQ ID NO: | DNA VH | CAAGTCCAACTCGTCCAGTCGGGAGCAGAAGTCAAG |
| 2114 | | AAGCCCGGATCATCCGTGAAAGTGTCCTGCAAAGCCT |
| | | CAGGCTACACCTTTACCAACTACAACTTGCACTGGGT |
| | | CAGACAGGCCCCGGGACAGGGCCTGGAGTGGATGGG |
| | | CGCCATCTACCCCGGAAACTATGACACCTCGTACAAC |
| | | CAGAAGTTCAAGGGTCGCGTGACTATCACGGCTGAC |
| | | AAGTCCACTAGCACCGCGTACATGGAACTTTCCTCAC |
| | | TGCGGTCCGAGGATACTGCGGTGTACTACTGCGCCCG |
| | | GGTGGACTTCGGACACTCGAGATATTGGTACTTCGAT |
| | | GTCTGGGGACAGGGGACCACCGTGACTGTGTCCTCC |
| SEQ ID NO: | LCDR1 | RATSSVSSMN |
| 2030 (Kabat) | | |
| SEQ ID NO: | LCDR2 | ATSNLAS |
| 2031 (Kabat) | | |
| SEQ ID NO: | LCDR3 | QQWTFNPPT |
| 2032 (Kabat) | | |
| SEQ ID NO: | LCDR1 | TSSVSS |
| 2033 (Chothia) | | |
| SEQ ID NO: | LCDR2 | ATS |
| 2034 (Chothia) | | |
| SEQ ID NO: | LCDR3 | WTFNPP |
| 2035 (Chothia) | | |
| SEQ ID NO: | LCDR1 | SSVSS |
| 2036 (IMGT) | | |
| SEQ ID NO: | LCDR2 | ATS |
| 2034 (IMGT) | | |
| SEQ ID NO: | LCDR3 | QQWTFNPPT |
| 2032 (IMGT) | | |
| SEQ ID NO: | LCDR1 | RATSSVSSMN |
| 2030 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWTFNPPT |
| 2032 (Combined | | |
| Chothia and | | |
| Kabat | | |
| SEQ ID NO: | VL | AIRMTQSPFSLSASVGDRVTITCRASGNIHNYLAWYQQ |
| 2095 | | KPAKAPKLFIYNTKTLADGVPSRFSGSGSGTDYTLTIS |
| | | SLQPEDFATYYCQHFWSSPWTFGGGTKVEIK |
| SEQ ID NO: | DNA VL | GAAATTGTGCTGACCCAGTCTCCCGCAACCCTGTCCC |
| 2115 | | TGAGCCCTGGAGAGCGCGCCACCCTGTCCTGCCGGGC |
| | | CACATCCTCCGTGTCGTCCATGAACTGGTACCAGCAG |
| | | AAGCCCGGCCAAGCCCCGAGGCCTCTGATTCATGCTA |
| | | CCTCAAATCTGGCCAGCGGAATCCCGGCGCGCTTCTC |
| | | CGGCTCGGGCAGCGGTACTGACTACACTCTCACCATC |
| | | TCGTCCCTCGAACCGGAGGACGCCGCCGTCTACTACT |
| | | GTCAGCAGTGGACCTTCAACCCACCTACTTTCGGACA |
| | | AGGGACCAAGCTGGAGATCAAG |
| SEQ ID NO: | Linker | GGGSGGGGSGGGGSGGGGS |
| 2116 | | |
| SEQ ID NO: | scFv (VH- | QVQLVQSGAEVKKPGASVKVSCKASGYTFTNYWMHW |
| 2097 | linker-VL) | VRQAPGQGLEWMGFITPTTGYPEYNQKFKDRVTMTAD |
| | | KSTSTAYMELSSLRSEDTAVYYCARRKVGKGVYYALD |
| | | YWGQGTTVTVSSGGGGSGGGGSGGGGSGGGGSAIRMT |
| | | QSPFSLSASVGDRVTITCRASGNIHNYLAWYQQKPAKA |
| | | PKLFIYNTKTLADGVPSRFSGSGSGTDYTLTISSLQP |
| | | EDFATYYCQHFWSSPWTFGGGTKVEIK |
| SEQ ID NO: | DNA scFv | CAAGTCCAACTCGTCCAGTCGGGAGCAGAAGTCAAG |
| 2117 | (VH-linker- | AAGCCCGGATCATCCGTGAAAGTGTCCTGCAAAGCCT |
| | VL) | CAGGCTACACCTTTACCAACTACAACTTGCACTGGGT |
| | | CAGACAGGCCCCGGGACAGGGCCTGGAGTGGATGGG |
| | | CGCCATCTACCCCGGAAACTATGACACCTCGTACAAC |
| | | CAGAAGTTCAAGGGTCGCGTGACTATCACGGCTGAC |
| | | AAGTCCACTAGCACCGCGTACATGGAACTTTCCTCAC |
| | | TGCGGTCCGAGGATACTGCGGTGTACTACTGCGCCCG |
| | | GGTGGACTTCGGACACTCGAGATATTGGTACTTCGAT |
| | | GTCTGGGGACAGGGGACCACCGTGACTGTGTCCTCCG |
| | | GGGGCGGTGGCAGCGGGGGAGGCGGAAGCGGCGGA |
| | | GGGGGTTCCGGGGGTGGAGGAAGCGAAATTGTGCTG |
| | | ACCCAGTCTCCCGCAACCCTGTCCCTGAGCCCTGGAG |
| | | AGCGCGCCACCCTGTCCTGCCGGGCCACATCCTCCGT |
| | | GTCGTCCATGAACTGGTACCAGCAGAAGCCCGGCCA |
| | | AGCCCCGAGGCCTCTGATTCATGCTACCTCAAATCTG |
| | | GCCAGCGGAATCCCGGCGCGCTTCTCCGGCTCGGGCA |
| | | GCGGTACTGACTACACTCTCACCATCTCGTCCCTCGA |
| | | ACCGGAGGACGCCGCCGTCTACTACTGTCAGCAGTG |
| | | GACCTTCAACCCACCTACTTTCGGACAAGGGACCAAG |
| | | CTGGAGATCAAG |
| SEQ ID NO: | Full CAR | MALPVTALLLPLALLLHAARPQVQLVQSGAEVKKPGSS |
| 2118 | amino acid | VKVSCKASGYTFTNYNLHWVRQAPGQGLEWMGAIYP |
| | sequence | GNYDTSYNQKFKGRVTITADKSTSTAYMELSSLRSEDT |
| | | AVYYCARVDFGHSRYWYFDVWGQGTTVTVSSGGGGS |
| | | GGGGSGGGGSGGGGSEIVLTQSPATLSLSPGERATLSCR |
| | | ATSSVSSMNWYQQKPGQAPRPLIHATSNLASGIPARFSG |
| | | SGSGTDYTLTISSLEPEDAAVYYCQQWTFNPPTFGQGTK |
| | | LEIKTTTPAPRPPTPAPTIASQPLSLRPEACRPAAGGAVH |
| | | TRGLDFACDIYIWAPLAGTCGVLLLSLVITLYCKRGRKK |
| | | LLYIFKQPFMRPVQTTQEEDGCSCRFPEEEEGGCELRVK |
| | | FSRSADAPAYQQGQNQLYNELNLGRREEYDVLDKRRG |
| | | RDPEMGGKPRRKNPQEGLYNELQKDKMAEAYSEIGMK |
| | | GERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2119 | nucleic acid | CTCTTCTGCTCCACGCCGCTCGGCCCCAAGTCCAACT |
| | sequence | CGTCCAGTCGGGAGCAGAAGTCAAGAAGCCCGGATC |
| | | ATCCGTGAAAGTGTCCTGCAAAGCCTCAGGCTACACC |
| | | TTTACCAACTACAACTTGCACTGGGTCAGACAGGCCC |
| | | CGGGACAGGGCCTGGAGTGGATGGGCGCCATCTACC |
| | | CCGGAAACTATGACACCTCGTACAACCAGAAGTTCA |
| | | AGGGTCGCGTGACTATCACGGCTGACAAGTCCACTA |
| | | GCACCGCGTACATGGAACTTTCCTCACTGCGGTCCGA |
| | | GGATACTGCGGTGTACTACTGCGCCCGGGTGGACTTC |
| | | GGACACTCGAGATATTGGTACTTCGATGTCTGGGGAC |
| | | AGGGGACCACCGTGACTGTGTCCTCCGGGGGCGGTG |
| | | GCAGCGGGGGAGGCGGAAGCGGCGGAGGGGGTTCC |
| | | GGGGGTGGAGGAAGCGAAATTGTGCTGACCCAGTCT |
| | | CCCGCAACCCTGTCCCTGAGCCCTGGAGAGCGCGCCA |
| | | CCCTGTCCTGCCGGGCCACATCCTCCGTGTCGTCCAT |
| | | GAACTGGTACCAGCAGAAGCCCGGCCAAGCCCCGAG |
| | | GCCTCTGATTCATGCTACCTCAAATCTGGCCAGCGGA |
| | | ATCCCGGCGCGCTTCTCCGGCTCGGGCAGCGGTACTG |
| | | ACTACACTCTCACCATCTCGTCCCTCGAACCGGAGGA |
| | | CGCCGCCGTCTACTACTGTCAGCAGTGGACCTTCAAC |
| | | CCACCTACTTTCGGACAAGGGACCAAGCTGGAGATC |
| | | AAGACCACTACCCCAGCACCGAGGCCACCCACCCCG |
| | | GCTCCTACCATCGCCTCCCAGCCTCTGTCCCTGCGTCC |
| | | GGAGGCATGTAGACCCGCAGCTGGTGGGGCCGTGCA |
| | | TACCCGGGGTCTTGACTTCGCCTGCGATATCTACATT |
| | | TGGGCCCCTCTGGCTGGTACTTGCGGGGTCCTGCTGC |
| | | TTTCACTCGTGATCACTCTTTACTGTAAGCGCGGTCG |
| | | GAAGAAGCTGCTGTACATCTTTAAGCAACCCTTCATG |
| | | AGGCCTGTGCAGACTACTCAAGAGGAGGACGGCTGT |
| | | TCATGCCGGTTCCCAGAGGAGGAGGAAGGCGGCTGC |
| | | GAACTGCGCGTGAAATTCAGCCGCAGCGCAGATGCT |
| | | CCAGCCTACCAGCAGGGGCAGAACCAGCTCTACAAC |
| | | GAACTCAATCTTGGTCGGAGAGAGGAGTACGACGTG |
| | | CTGGACAAGCGGAGAGGACGGGACCCAGAAATGGGC |
| | | GGGAAGCCGCGCAGAAAGAATCCCCAAGAGGGCCTG |
| | | TACAACGAGCTCCAAAAGGATAAGATGGCAGAAGCC |
| | | TATAGCGAGATTGGTATGAAAGGGGAACGCAGAAGA |
| | | GGCAAAGGCCACGACGGACTGTACCAGGGACTCAGC |
| | | ACCGCCACCAAGGACACCTATGACGCTCTTCACATGC |
| | | AGGCCCTGCCGCCTCGG |
| CD20-C3H4 | | |
| SEQ ID NO: | HCDR1 | NYNLH |
| 2019 (Kabat) | | |
| SEQ ID NO: | HCDR2 | AIYPGNYDTSYNQKFKG |
| 2020 (Kabat) | | |
| SEQ ID NO: | HCDR3 | VDFGHSRYWYFDV |
| 2021 (Kabat) | | |
| SEQ ID NO: | HCDR1 | GYTFTNY |
| 2022 (Chothia) | | |
| SEQ ID NO: | HCDR2 | YPGNYD |
| 2023 (Chothia) | | |
| SEQ ID NO: | HCDR3 | VDFGHSRYWYFDV |
| 2021 (Chothia) | | |
| SEQ ID NO: | HCDR1 | GYTFTNYN |
| 2024 (IMGT) | | |
| SEQ ID NO: | HCDR2 | IYPGNYDT |
| 2025 (IMGT) | | |
| SEQ ID NO: | HCDR3 | ARVDFGHSRYWYFDV |
| 2026 (IMGT) | | |
| SEQ ID NO: | HCDR1 | GYTFTNYNLH |
| 2027 (Combined | | |
| Chothia and | | |
| Kabat) | | |
| SEQ ID NO: | HCDR2 | AIYPGNYDTSYNQKFKG |
| 2020 (Combined | | |
| Chothia and | | |
| Kabat) | | |
| SEQ ID NO: | HCDR3 | VDFGHSRYWYFDV |
| 2021 (Combined | | |
| Chothia and | | |
| Kabat) | | |
| SEQ ID NO: | VH | QVQLVQSGAEVKKPGSSVKVSCKASGYTFTNYNLHWV |
| 2120 | | RQAPGQGLEWMGAIYPGNYDTSYNQKFKGRVTITADK |
| | | STSTAYMELSSLRSEDTAVYYCARVDFGHSRYWYFDV |
| | | WGQGTTVTVSS |
| SEQ ID NO: | DNA VH | CAAGTCCAACTCGTCCAGTCGGGAGCAGAAGTCAAG |
| 2114 | | AAGCCCGGATCATCCGTGAAAGTGTCCTGCAAAGCCT |
| | | CAGGCTACACCTTTACCAACTACAACTTGCACTGGGT |
| | | CAGACAGGCCCCGGGACAGGGCCTGGAGTGGATGGG |
| | | CGCCATCTACCCCGGAAACTATGACACCTCGTACAAC |
| | | CAGAAGTTCAAGGGTCGCGTGACTATCACGGCTGAC |
| | | AAGTCCACTAGCACCGCGTACATGGAACTTTCCTCAC |
| | | TGCGGTCCGAGGATACTGCGGTGTACTACTGCGCCCG |
| | | GGTGGACTTCGGACACTCGAGATATTGGTACTTCGAT |
| | | GTCTGGGGACAGGGGACCACCGTGACTGTGTCCTCC |
| SEQ ID NO: | LCDR1 | RATSSVSSMN |
| 2030 (Kabat) | | |
| SEQ ID NO: | LCDR2 | ATSNLAS |
| 2031 (Kabat) | | |
| SEQ ID NO: | LCDR3 | QQWTFNPPT |
| 2032 (Kabat) | | |
| SEQ ID NO: | LCDR1 | TSSVSS |
| 2033 (Chothia) | | |
| SEQ ID NO: | LCDR2 | ATS |
| 2034 (Chothia) | | |
| SEQ ID NO: | LCDR3 | WTFNPP |
| 2035 (Chothia) | | |
| SEQ ID NO: | LCDR | SSVSS |
| 2036 (IMGT) | | |
| SEQ ID NO: | LCDR2 | ATS |
| 2034 (IMGT) | | |
| SEQ ID NO: | LCDR3 | QQWTFNPPT |
| 2032 (IMGT) | | |
| SEQ ID NO: | LCDR1 | RATSSVSSMN |
| 2030 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWTFNPPT |
| 2032 (Combined | | |
| Chothia and | | |
| Kabat) | | |
| SEQ ID NO: | VL | EIVLTQSPATLSLSPGERATLSCRATSSVSSMNWYQQKP |
| 2108 | | GQAPRPLIHATSNLASGIPARFSGSGSGTDYTLTISSLE |
| | | PEDAAVYYCQQWTFNPPTFGQGTKLEIK |
| SEQ ID NO: | DNA VL | GAAATTGTGCTGACCCAGTCTCCCGCAACCCTGTCCC |
| 2115 | | TGAGCCCTGGAGAGCGCGCCACCCTGTCCTGCCGGGC |
| | | CACATCCTCCGTGTCGTCCATGAACTGGTACCAGCAG |
| | | AAGCCCGGCCAAGCCCCGAGGCCTCTGATTCATGCTA |
| | | CCTCAAATCTGGCCAGCGGAATCCCGGCGCGCTTCTC |
| | | CGGCTCGGGCAGCGGTACTGACTACACTCTCACCATC |
| | | TCGTCCCTCGAACCGGAGGACGCCGCCGTCTACTACT |
| | | GTCAGCAGTGGACCTTCAACCCACCTACTTTCGGACA |
| | | AGGGACCAAGCTGGAGATCAAG |
| SEQ ID NO: | Linker | GGGGSGGGGSGGGGSGGGGS |
| 1010 | | |
| SEQ ID NO: | scFv (VH- | QVQLVQSGAEVKKPGSSVKVSCKASGYTFTNYNLHWV |
| 2121 | linker-VL) | RQAPGQGLEWMGAIYPGNYDTSYNQKFKGRVTITADK |
| | | STSTAYMELSSLRSEDTAVYYCARVDFGHSRYWYFDV |
| | | WGQGTTVTVSSGGGGSGGGGSGGGGSGGGGSEIVLTQ |
| | | SPATLSLSPGERATLSCRATSSVSSMNWYQQKPGQAPR |
| | | PLIHATSNLASGIPARFSGSGSGTDYTLTISSLEPEDA |
| | | AVYYCQQWTFNPPTFGQGTKLEIK |
| SEQ ID NO: | DNA scFv | CAAGTCCAACTCGTCCAATCCGGCGCAGAAGTCAAG |
| 2122 | (VH-linker- | AAACCAGGATCGTCCGTGAAAGTGTCCTGCAAGGCG |
| | VL | TCCGGGTACACCTTCACTAATTACAACCTCCACTGGG |
| | | TCAGACAGGCCCCAGGACAGGGCCTGGAATGGATGG |
| | | GCGCCATCTACCCTGGAAACTACGATACCTCGTACAA |
| | | CCAGAAGTTCAAGGGCCGCGTGACTATTACCGCCGA |
| | | CAAGAGCACCTCCACCGCCTATATGGAACTGTCGTCC |
| | | CTGCGGTCCGAGGACACTGCCGTGTACTACTGTGCAA |
| | | GGGTGGACTTCGGTCACTCCCGGTATTGGTACTTCGA |
| | | CGTCTGGGGACAGGGGACCACTGTGACCGTGTCGTC |
| | | GGGAGGCGGTGGAAGCGGCGGTGGCGGAAGCGGAG |
| | | GCGGCGGATCAGGGGGCGGAGGAAGCGACATTCAGC |
| | | TTACCCAGTCACCGTCCTTCCTGAGCGCCTCCGTGGG |
| | | AGATCGCGTGACCATCACATGCCGCGCCACTTCCTCG |
| | | GTGTCCTCCATGAACTGGTACCAGCAGAAGCCCGGA |
| | | AAGGCTCCTAAGCCTCTGATCCATGCGACCTCCAACT |
| | | TGGCTTCCGGGGTGCCGTCACGGTTCAGCGGCAGCGG |
| | | TTCAGGAACTGAGTACACCCTGACTATTAGCTCTCTC |
| | | CAACCCGAGGACTTCGCCACCTACTACTGCCAGCAGT |
| | | GGACCTTCAACCCGCCCACGTTTGGGCAGGGTACCAA |
| | | GCTGGAGATCAAG |
| SEQ ID NO: | Full CAR | MALPVTALLLPLALLLHAARPQVQLVQSGAEVKKPGSS |
| 2123 | amino acid | VKVSCKASGYTFTNYNLHWVRQAPGQGLEWMGAIYP |
| | sequence | GNYDTSYNQKFKGRVTITADKSTSTAYMELSSLRSEDT |
| | | AVYYCARVDFGHSRYWYFDVWGQGTTVTVSSGGGGS |
| | | GGGGSGGGGSGGGGSDIQLTQSPSFLSASVGDRVTITCR |
| | | ATSSVSSMNWYQQKPGKAPKPLIHATSNLASGVPSRFS |
| | | GSGSGTEYTLTISSLQPEDFATYYCQQWTFNPPTFGQGT |
| | | KLEIKTTTPAPRPPTPAPTIASQPLSLRPEACRPAAGGAV |
| | | HTRGLDFACDIYIWAPLAGTCGVLLLSLVITLYCKRGRK |
| | | KLLYIFKQPFMRPVQTTQEEDGCSCRFPEEEEGGCELRV |
| | | KFSRSADAPAYQQGQNQLYNELNLGRREEYDVLDKRR |
| | | GRDPEMGGKPRRKNPQEGLYNELQKDKMAEAYSEIGM |
| | | KGERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2124 | nucleic acid | CTCTTCTGCTCCACGCCGCTCGGCCCCAAGTCCAACT |
| | sequence | CGTCCAATCCGGCGCAGAAGTCAAGAAACCAGGATC |
| | | GTCCGTGAAAGTGTCCTGCAAGGCGTCCGGGTACACC |
| | | TTCACTAATTACAACCTCCACTGGGTCAGACAGGCCC |
| | | CAGGACAGGGCCTGGAATGGATGGGCGCCATCTACC |
| | | CTGGAAACTACGATACCTCGTACAACCAGAAGTTCA |
| | | AGGGCCGCGTGACTATTACCGCCGACAAGAGCACCT |
| | | CCACCGCCTATATGGAACTGTCGTCCCTGCGGTCCGA |
| | | GGACACTGCCGTGTACTACTGTGCAAGGGTGGACTTC |
| | | GGTCACTCCCGGTATTGGTACTTCGACGTCTGGGGAC |
| | | AGGGGACCACTGTGACCGTGTCGTCGGGAGGCGGTG |
| | | GAAGCGGCGGTGGCGGAAGCGGAGGCGGCGGATCA |
| | | GGGGGCGGAGGAAGCGACATTCAGCTTACCCAGTCA |
| | | CCGTCCTTCCTGAGCGCCTCCGTGGGAGATCGCGTGA |
| | | CCATCACATGCCGCGCCACTTCCTCGGTGTCCTCCAT |
| | | GAACTGGTACCAGCAGAAGCCCGGAAAGGCTCCTAA |
| | | GCCTCTGATCCATGCGACCTCCAACTTGGCTTCCGGG |
| | | GTGCCGTCACGGTTCAGCGGCAGCGGTTCAGGAACT |
| | | GAGTACACCCTGACTATTAGCTCTCTCCAACCCGAGG |
| | | ACTTCGCCACCTACTACTGCCAGCAGTGGACCTTCAA |
| | | CCCGCCCACGTTTGGGCAGGGTACCAAGCTGGAGAT |
| | | CAAGACCACTACCCCAGCACCGAGGCCACCCACCCC |
| | | GGCTCCTACCATCGCCTCCCAGCCTCTGTCCCTGCGT |
| | | CCGGAGGCATGTAGACCCGCAGCTGGTGGGGCCGTG |
| | | CATACCCGGGGTCTTGACTTCGCCTGCGATATCTACA |
| | | TTTGGGCCCCTCTGGCTGGTACTTGCGGGGTCCTGCT |
| | | GCTTTCACTCGTGATCACTCTTTACTGTAAGCGCGGT |
| | | CGGAAGAAGCTGCTGTACATCTTTAAGCAACCCTTCA |
| | | TGAGGCCTGTGCAGACTACTCAAGAGGAGGACGGCT |
| | | GTTCATGCCGGTTCCCAGAGGAGGAGGAAGGCGGCT |
| | | GCGAACTGCGCGTGAAATTCAGCCGCAGCGCAGATG |
| | | CTCCAGCCTACCAGCAGGGGCAGAACCAGCTCTACA |
| | | ACGAACTCAATCTTGGTCGGAGAGAGGAGTACGACG |
| | | TGCTGGACAAGCGGAGAGGACGGGACCCAGAAATGG |
| | | GCGGGAAGCCGCGCAGAAAGAATCCCCAAGAGGGCC |
| | | TGTACAACGAGCTCCAAAAGGATAAGATGGCAGAAG |
| | | CCTATAGCGAGATTGGTATGAAAGGGGAACGCAGAA |
| | | GAGGCAAAGGCCACGACGGACTGTACCAGGGACTCA |
| | | GCACCGCCACCAAGGACACCTATGACGCTCTTCACAT |
| | | GCAGGCCCTGCCGCCTCGG |
| CD20-C5H2 | | |
| SEQ ID NO: | HCDR1 | SYNMH |
| 2043 (Kabat) | | |
| SEQ ID NO: | HCDR2 | AIYPGNGDTSYNPKFKG |
| 2044 (Kabat) | | |
| SEQ ID NO: | HCDR3 | SYFYGSSSWYFDV |
| 2045 (Kabat) | | |
| SEQ ID NO: | HCDR1 | GYTFTSY |
| 2046 (Chothia) | | |
| SEQ ID NO: | HCDR2 | YPGNGD |
| 2047 (Chothia) | | |
| SEQ ID NO: | HCDR3 | SYFYGSSSWYFDV |
| 2045 (Chothia) | | |
| SEQ ID NO: | HCDR1 | GYTFTSYN |
| 2048 (IMGT) | | |
| SEQ ID NO: | HCDR2 | IYPGNGDT |
| 2049 (IMGT) | | |
| SEQ ID NO: | HCDR3 | ARSYFYGSSSWYFDV |
| 2050 (IMGT) | | |
| SEQ ID NO: | HCDR1 | GYTFTSYNMH |
| 2051 (Combined | | |
| Chothia and | | |
| Kabat) | | |
| SEQ ID NO: | HCDR2 | AIYPGNGDTSYNPKFKG |
| 2044 (Combined | | |
| Chothia and | | |
| Kabat) | | |
| SEQ ID NO: | HCDR3 | SYFYGSSSWYFDV |
| 2045 (Combined | | |
| Chothia and | | |
| Kabat) | | |
| SEQ ID NO: | VH | QVQLVQSGAEVKKPGASVKVSCKASGYTFTSYNMHW |
| 2052 | | VRQAPGQGLEWMGAIYPGNGDTSYNPKFKGRVTMTA |
| | | DKSTRTAYMELSSLRSEDTAVYYCARSYFYGSSSWYFD |
| | | VWGQGTTVTVSS |
| SEQ ID NO: | DNA VH | CAAGTCCAACTCGTCCAGTCAGGAGCAGAAGTCAAG |
| 2125 | | AAACCTGGAGCTTCCGTGAAAGTGTCGTGCAAGGCCT |
| | | CCGGCTACACCTTCACCTCTTACAACATGCACTGGGT |
| | | CAGACAGGCCCCTGGTCAAGGACTGGAATGGATGGG |
| | | AGCGATCTACCCGGGCAACGGAGACACTTCGTACAA |
| | | CCCCAAGTTCAAGGGACGGGTCACTATGACCGCCGA |
| | | TAAGAGCACGCGCACCGCGTACATGGAACTGAGCAG |
| | | CCTGCGCTCCGAGGACACTGCCGTGTATTACTGCGCG |
| | | AGGAGCTACTTCTACGGATCATCGTCGTGGTACTTCG |
| | | ACGTCTGGGGCCAGGGCACCACCGTGACCGTGTCATC |
| | | C |
| SEQ ID NO: | LCDR1 | RASSSVSSMH |
| 2054 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDR1 | SSSVSS |
| 2056 (Chothia) | | |
| SEQ ID NO | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVSS |
| 2036 (IMGT) | | |
| SEQ ID NO: | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVSSMH |
| 2054 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 (Combined | | |
| Chothia and | | |
| Kabat) | | |
| SEQ ID NO: | VL | DIQLTQSPSFLSASVGDRVTITCRASSSVSSMHWYQQKP |
| 2126 | | GKAPKPLIFATSNLASGVPSRFSGSGSGTEYTLTISSLQ |
| | | PEDFATYYCQQWIFNPPTFGGGTKVEIK |
| SEQ ID NO: | DNA VL | GATATTCAGCTGACCCAGAGCCCGTCATTCCTGTCCG |
| 2127 | | CCTCCGTGGGAGACAGAGTGACCATCACTTGTCGGGC |
| | | CAGCTCCTCGGTGTCCTCCATGCATTGGTATCAGCAG |
| | | AAGCCTGGGAAGGCTCCCAAGCCCCTCATCTTCGCCA |
| | | CATCAAATCTTGCCTCCGGGGTGCCAAGCCGGTTCTC |
| | | CGGGAGCGGCTCCGGTACTGAGTACACTCTGACCATT |
| | | TCCTCCTTGCAACCCGAGGACTTTGCCACCTACTACT |
| | | GCCAGCAGTGGATCTTTAACCCGCCGACCTTCGGAGG |
| | | AGGAACCAAAGTGGAGATCAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTSYNMHW |
| 2128 | linker-VL) | VRQAPGQGLEWMGAIYPGNGDTSYNPKFKGRVTMTA |
| | | DKSTRTAYMELSSLRSEDTAVYYCARSYFYGSSSWYFD |
| | | VWGQGTTVTVSSGGGGSGGGGSGGGGSGGGGSDIQLT |
| | | QSPSFLSASVGDRVTITCRASSSVSSMHWYQQKPGKAP |
| | | KPLIFATSNLASGVPSRFSGSGSGTEYTLTISSLQPED |
| | | FATYYCQQWIFNPPTFGGGTKVEIK |
| SEQ ID NO: | DNA scFv | CAAGTCCAACTCGTCCAGTCAGGAGCAGAAGTCAAG |
| 2129 | (VH-linker- | AAACCTGGAGCTTCCGTGAAAGTGTCGTGCAAGGCCT |
| | VL) | CCGGCTACACCTTCACCTCTTACAACATGCACTGGGT |
| | | CAGACAGGCCCCTGGTCAAGGACTGGAATGGATGGG |
| | | AGCGATCTACCCGGGCAACGGAGACACTTCGTACAA |
| | | CCCCAAGTTCAAGGGACGGGTCACTATGACCGCCGA |
| | | TAAGAGCACGCGCACCGCGTACATGGAACTGAGCAG |
| | | CCTGCGCTCCGAGGACACTGCCGTGTATTACTGCGCG |
| | | AGGAGCTACTTCTACGGATCATCGTCGTGGTACTTCG |
| | | ACGTCTGGGGCCAGGGCACCACCGTGACCGTGTCATC |
| | | CGGTGGCGGAGGATCGGGGGGCGGAGGAAGCGGCG |
| | | GGGGGGGCTCCGGCGGTGGAGGCTCGGATATTCAGC |
| | | TGACCCAGAGCCCGTCATTCCTGTCCGCCTCCGTGGG |
| | | AGACAGAGTGACCATCACTTGTCGGGCCAGCTCCTCG |
| | | GTGTCCTCCATGCATTGGTATCAGCAGAAGCCTGGGA |
| | | AGGCTCCCAAGCCCCTCATCTTCGCCACATCAAATCT |
| | | TGCCTCCGGGGTGCCAAGCCGGTTCTCCGGGAGCGGC |
| | | TCCGGTACTGAGTACACTCTGACCATTTCCTCCTTGC |
| | | AACCCGAGGACTTTGCCACCTACTACTGCCAGCAGTG |
| | | GATCTTTAACCCGCCGACCTTCGGAGGAGGAACCAA |
| | | AGTGGAGATCAAG |
| SEQ ID NO: | Full CAR | MALPVTALLLPLALLLHAARPQVQLVQSGAEVKKPGAS |
| 2130 | amino acid | VKVSCKASGYTFTSYNMHWVRQAPGQGLEWMGAIYP |
| | sequence | GNGDTSYNPKFKGRVTMTADKSTRTAYMELSSLRSEDT |
| | | AVYYCARSYFYGSSSWYFDVWGQGTTVTVSSGGGGSG |
| | | GGGSGGGGSGGGGSDIQLTQSPSFLSASVGDRVTITCRA |
| | | SSSVSSMHWYQQKPGKAPKPLIFATSNLASGVPSRFSGS |
| | | GSGTEYTLTISSLQPEDFATYYCQQWIFNPPTFGGGTKV |
| | | EIKTTTPAPRPPTPAPTIASQPLSLRPEACRPAAGGAVHT |
| | | RGLDFACDIYIWAPLAGTCGVLLLSLVITLYCKRGRKKL |
| | | LYIFKQPFMRPVQTTQEEDGCSCRFPEEEEGGCELRVKF |
| | | SRSADAPAYQQGQNQLYNELNLGRREEYDVLDKRRGR |
| | | DPEMGGKPRRKNPQEGLYNELQKDKMAEAYSEIGMKG |
| | | ERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2131 | nucleic acid | CTCTTCTGCTCCACGCCGCTCGGCCCCAAGTCCAACT |
| | sequence | CGTCCAGTCAGGAGCAGAAGTCAAGAAACCTGGAGC |
| | | TTCCGTGAAAGTGTCGTGCAAGGCCTCCGGCTACACC |
| | | TTCACCTCTTACAACATGCACTGGGTCAGACAGGCCC |
| | | CTGGTCAAGGACTGGAATGGATGGGAGCGATCTACC |
| | | CGGGCAACGGAGACACTTCGTACAACCCCAAGTTCA |
| | | AGGGACGGGTCACTATGACCGCCGATAAGAGCACGC |
| | | GCACCGCGTACATGGAACTGAGCAGCCTGCGCTCCG |
| | | AGGACACTGCCGTGTATTACTGCGCGAGGAGCTACTT |
| | | CTACGGATCATCGTCGTGGTACTTCGACGTCTGGGGC |
| | | CAGGGCACCACCGTGACCGTGTCATCCGGTGGCGGA |
| | | GGATCGGGGGGCGGAGGAAGCGGCGGGGGGGGCTC |
| | | CGGCGGTGGAGGCTCGGATATTCAGCTGACCCAGAG |
| | | CCCGTCATTCCTGTCCGCCTCCGTGGGAGACAGAGTG |
| | | ACCATCACTTGTCGGGCCAGCTCCTCGGTGTCCTCCA |
| | | TGCATTGGTATCAGCAGAAGCCTGGGAAGGCTCCCA |
| | | AGCCCCTCATCTTCGCCACATCAAATCTTGCCTCCGG |
| | | GGTGCCAAGCCGGTTCTCCGGGAGCGGCTCCGGTACT |
| | | GAGTACACTCTGACCATTTCCTCCTTGCAACCCGAGG |
| | | ACTTTGCCACCTACTACTGCCAGCAGTGGATCTTTAA |
| | | CCCGCCGACCTTCGGAGGAGGAACCAAAGTGGAGAT |
| | | CAAGACCACTACCCCAGCACCGAGGCCACCCACCCC |
| | | GGCTCCTACCATCGCCTCCCAGCCTCTGTCCCTGCGT |
| | | CCGGAGGCATGTAGACCCGCAGCTGGTGGGGCCGTG |
| | | CATACCCGGGGTCTTGACTTCGCCTGCGATATCTACA |
| | | TTTGGGCCCCTCTGGCTGGTACTTGCGGGGTCCTGCT |
| | | GCTTTCACTCGTGATCACTCTTTACTGTAAGCGCGGT |
| | | CGGAAGAAGCTGCTGTACATCTTTAAGCAACCCTTCA |
| | | TGAGGCCTGTGCAGACTACTCAAGAGGAGGACGGCT |
| | | GTTCATGCCGGTTCCCAGAGGAGGAGGAAGGCGGCT |
| | | GCGAACTGCGCGTGAAATTCAGCCGCAGCGCAGATG |
| | | CTCCAGCCTACCAGCAGGGGCAGAACCAGCTCTACA |
| | | ACGAACTCAATCTTGGTCGGAGAGAGGAGTACGACG |
| | | TGCTGGACAAGCGGAGAGGACGGGACCCAGAAATGG |
| | | GCGGGAAGCCGCGCAGAAAGAATCCCCAAGAGGGCC |
| | | TGTACAACGAGCTCCAAAAGGATAAGATGGCAGAAG |
| | | CCTATAGCGAGATTGGTATGAAAGGGGAACGCAGAA |
| | | GAGGCAAAGGCCACGACGGACTGTACCAGGGACTCA |
| | | GCACCGCCACCAAGGACACCTATGACGCTCTTCACAT |
| | | GCAGGCCCTGCCGCCTCGG |
| CD20-C5H3 | | |
| SEQ ID NO: | HCDR1 | SYNMH |
| 2043 (Kabat) | | |
| SEQ ID NO: | HCDR2 | AIYPGNGDTSYNPKFKG |
| 2044 (Kabat) | | |
| SEQ ID NO: | HCDR3 | SYFYGSSSWYFDV |
| 2045 (Kabat) | | |
| SEQ ID NO: | HCDR1 | GYTFTSY |
| 2046 (Chothia) | | |
| SEQ ID NO: | HCDR2 | YPGNGD |
| 2047 (Chothia) | | |
| SEQ ID NO: | HCDR3 | SYFYGSSSWYFDV |
| 2045 (Chothia) | | |
| SEQ ID NO: | HCDR1 | GYTFTSYN |
| 2048 (IMGT) | | |
| SEQ ID NO: | HCDR2 | IYPGNGDT |
| 2049 (IMGT) | | |
| SEQ ID NO: | HCDR3 | ARSYFYGSSSWYFDV |
| 2050 (IMGT) | | |
| SEQ ID NO: | HCDR1 | GYTFTSYNMH |
| 2051 (Combined | | |
| Chothia and | | |
| Kabat) | | |
| SEQ ID NO: | HCDR2 | AIYPGNGDTSYNPKFKG |
| 2044 (Combined | | |
| Chothia and | | |
| Kabat) | | |
| SEQ ID NO: | HCDR3 | SYFYGSSSWYFDV |
| 2045 (Combined | | |
| Chothia and | | |
| Kabat) | | |
| SEQ ID NO: | VH | QVQLVQSGAEVKKPGSSVKVSCKASGYTFTSYNMHWV |
| 2132 | | RQAPGQGLEWMGAIYPGNGDTSYNPKFKGRVTITADK |
| | | STRTAYMELSSLRSEDTAVYYCARSYFYGSSSWYFDV |
| | | WGQGTTVTVSS |
| SEQ ID NO: | DNA VH | CAAGTGCAACTCGTCCAGTCCGGTGCAGAAGTCAAG |
| 2133 | | AAGCCTGGTTCATCGGTGAAAGTGTCCTGCAAAGCGT |
| | | CGGGCTACACCTTCACCTCGTACAACATGCACTGGGT |
| | | CCGCCAGGCCCCCGGACAAGGACTGGAATGGATGGG |
| | | TGCTATCTACCCCGGAAACGGAGATACCAGCTACAA |
| | | CCCCAAGTTCAAGGGACGCGTGACCATTACTGCCGAC |
| | | AAGTCCACAAGAACCGCCTACATGGAACTGTCCAGC |
| | | CTGAGATCCGAGGACACTGCGGTGTACTACTGTGCGA |
| | | GGTCCTACTTCTACGGGTCCTCCTCTTGGTACTTCGAC |
| | | GTCTGGGGACAGGGCACTACTGTGACCGTGTCCAGC |
| SEQ ID NO: | LCDR1 | RASSSVSSMH |
| 2054 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDR1 | SSSVSS |
| 2056 (Chothia) | | |
| SEQ ID NO: | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVSS |
| 2036 (IMGT) | | |
| SEQ ID NO: | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVSSMH |
| 2054 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 (Combined | | |
| Chothia and | | |
| Kabat) | | |
| SEQ ID NO: | VL | EIVLTQSPATLSLSPGERATLSCRASSSVSSMHWYQQKP |
| 2058 | | GQAPRPLIFATSNLASGIPARFSGSGSGTDYTLTISSLE |
| | | PEDAAVYYCQQWIFNPPTFGGGTKVEIK |
| SEQ ID NO: | DNA VL | GAGATCGTGCTGACGCAGTCGCCGGCCACCCTGAGC |
| 2134 | | CTTTCACCGGGAGAACGCGCCACTCTGTCATGCCGGG |
| | | CCAGCAGCTCCGTGTCCTCCATGCATTGGTACCAGCA |
| | | GAAGCCGGGGCAGGCCCCGCGGCCTCTCATCTTCGCC |
| | | ACCTCCAATCTGGCCTCCGGCATCCCTGCTCGGTTTA |
| | | GCGGAAGCGGCAGCGGAACTGACTATACCTTGACCA |
| | | TCTCCTCGCTGGAACCAGAGGATGCAGCCGTGTACTA |
| | | TTGCCAGCAGTGGATCTTCAACCCGCCAACCTTCGGC |
| | | GGCGGCACCAAGGTCGAGATTAAG |
| SEQ ID NO: | Linker | GGGGSGGGGSGGGGSGGGGS |
| 1010 | | |
| SEQ ID NO: | scFv (VH- | QVQLVQSGAEVKKPGSSVKVSCKASGYTFTSYNMHWV |
| 2135 | linker-VL) | RQAPGQGLEWMGAIYPGNGDTSYNPKFKGRVTITADK |
| | | STRTAYMELSSLRSEDTAVYYCARSYFYGSSSWYFDV |
| | | WGQGTTVTVSSGGGGSGGGGSGGGGSGGGGSEIVLTQ |
| | | SPATLSLSPGERATLSCRASSSVSSMHWYQQKPGQAPRP |
| | | LIFATSNLASGIPARFSGSGSGTDYTLTISSLEPEDAAVY |
| | | YCQQWIFNPPTFGGGTKVEIK |
| SEQ ID NO: | DNA scFv | CAAGTGCAACTCGTCCAGTCCGGTGCAGAAGTCAAG |
| 2136 | (VH-linker- | AAGCCTGGTTCATCGGTGAAAGTGTCCTGCAAAGCGT |
| | VL) | CGGGCTACACCTTCACCTCGTACAACATGCACTGGGT |
| | | CCGCCAGGCCCCCGGACAAGGACTGGAATGGATGGG |
| | | TGCTATCTACCCCGGAAACGGAGATACCAGCTACAA |
| | | CCCCAAGTTCAAGGGACGCGTGACCATTACTGCCGAC |
| | | AAGTCCACAAGAACCGCCTACATGGAACTGTCCAGC |
| | | CTGAGATCCGAGGACACTGCGGTGTACTACTGTGCGA |
| | | GGTCCTACTTCTACGGGTCCTCCTCTTGGTACTTCGAC |
| | | GTCTGGGGACAGGGCACTACTGTGACCGTGTCCAGC |
| | | GGGGGAGGCGGTAGCGGGGGGGGTGGATCGGGCGG |
| | | CGGCGGATCAGGAGGAGGAGGGTCCGAGATCGTGCT |
| | | GACGCAGTCGCCGGCCACCCTGAGCCTTTCACCGGGA |
| | | GAACGCGCCACTCTGTCATGCCGGGCCAGCAGCTCCG |
| | | TGTCCTCCATGCATTGGTACCAGCAGAAGCCGGGGCA |
| | | GGCCCCGCGGCCTCTCATCTTCGCCACCTCCAATCTG |
| | | GCCTCCGGCATCCCTGCTCGGTTTAGCGGAAGCGGCA |
| | | GCGGAACTGACTATACCTTGACCATCTCCTCGCTGGA |
| | | ACCAGAGGATGCAGCCGTGTACTATTGCCAGCAGTG |
| | | GATCTTCAACCCGCCAACCTTCGGCGGCGGCACCAAG |
| | | GTCGAGATTAAG |
| SEQ ID NO: | Full CAR | MALPVTALLLPLALLLHAARPQVQLVQSGAEVKKPGSS |
| 2137 | amino acid | VKVSCKASGYTFTSYNMHWVRQAPGQGLEWMGAIYP |
| | sequence | GNGDTSYNPKFKGRVTITADKSTRTAYMELSSLRSEDT |
| | | AVYYCARSYFYGSSSWYFDVWGQGTTVTVSSGGGGSG |
| | | GGGSGGGGSGGGGSEIVLTQSPATLSLSPGERATLSCRA |
| | | SSSVSSMHWYQQKPGQAPRPLIFATSNLASGIPARFSGS |
| | | GSGTDYTLTISSLEPEDAAVYYCQQWIFNPPTFGGGTKV |
| | | EIKTTTPAPRPPTPAPTIASQPLSLRPEACRPAAGGAVHT |
| | | RGLDFACDIYIWAPLAGTCGVLLLSLVITLYCKRGRKKL |
| | | LYIFKQPFMRPVQTTQEEDGCSCRFPEEEEGGCELRVKF |
| | | SRSADAPAYQQGQNQLYNELNLGRREEYDVLDKRRGR |
| | | DPEMGGKPRRKNPQEGLYNELQKDKMAEAYSEIGMKG |
| | | ERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2138 | nucleic acid | CTCTTCTGCTCCACGCCGCTCGGCCCCAAGTGCAACT |
| | sequence | CGTCCAGTCCGGTGCAGAAGTCAAGAAGCCTGGTTC |
| | | ATCGGTGAAAGTGTCCTGCAAAGCGTCGGGCTACAC |
| | | CTTCACCTCGTACAACATGCACTGGGTCCGCCAGGCC |
| | | CCCGGACAAGGACTGGAATGGATGGGTGCTATCTAC |
| | | CCCGGAAACGGAGATACCAGCTACAACCCCAAGTTC |
| | | AAGGGACGCGTGACCATTACTGCCGACAAGTCCACA |
| | | AGAACCGCCTACATGGAACTGTCCAGCCTGAGATCC |
| | | GAGGACACTGCGGTGTACTACTGTGCGAGGTCCTACT |
| | | TCTACGGGTCCTCCTCTTGGTACTTCGACGTCTGGGG |
| | | ACAGGGCACTACTGTGACCGTGTCCAGCGGGGGAGG |
| | | CGGTAGCGGGGGGGGTGGATCGGGCGGCGGCGGATC |
| | | AGGAGGAGGAGGGTCCGAGATCGTGCTGACGCAGTC |
| | | GCCGGCCACCCTGAGCCTTTCACCGGGAGAACGCGC |
| | | CACTCTGTCATGCCGGGCCAGCAGCTCCGTGTCCTCC |
| | | ATGCATTGGTACCAGCAGAAGCCGGGGCAGGCCCCG |
| | | CGGCCTCTCATCTTCGCCACCTCCAATCTGGCCTCCG |
| | | GCATCCCTGCTCGGTTTAGCGGAAGCGGCAGCGGAA |
| | | CTGACTATACCTTGACCATCTCCTCGCTGGAACCAGA |
| | | GGATGCAGCCGTGTACTATTGCCAGCAGTGGATCTTC |
| | | AACCCGCCAACCTTCGGCGGCGGCACCAAGGTCGAG |
| | | ATTAAGACCACTACCCCAGCACCGAGGCCACCCACC |
| | | CCGGCTCCTACCATCGCCTCCCAGCCTCTGTCCCTGC |
| | | GTCCGGAGGCATGTAGACCCGCAGCTGGTGGGGCCG |
| | | TGCATACCCGGGGTCTTGACTTCGCCTGCGATATCTA |
| | | CATTTGGGCCCCTCTGGCTGGTACTTGCGGGGTCCTG |
| | | CTGCTTTCACTCGTGATCACTCTTTACTGTAAGCGCG |
| | | GTCGGAAGAAGCTGCTGTACATCTTTAAGCAACCCTT |
| | | CATGAGGCCTGTGCAGACTACTCAAGAGGAGGACGG |
| | | CTGTTCATGCCGGTTCCCAGAGGAGGAGGAAGGCGG |
| | | CTGCGAACTGCGCGTGAAATTCAGCCGCAGCGCAGA |
| | | TGCTCCAGCCTACCAGCAGGGGCAGAACCAGCTCTA |
| | | CAACGAACTCAATCTTGGTCGGAGAGAGGAGTACGA |
| | | CGTGCTGGACAAGCGGAGAGGACGGGACCCAGAAAT |
| | | GGGCGGGAAGCCGCGCAGAAAGAATCCCCAAGAGG |
| | | GCCTGTACAACGAGCTCCAAAAGGATAAGATGGCAG |
| | | AAGCCTATAGCGAGATTGGTATGAAAGGGGAACGCA |
| | | GAAGAGGCAAAGGCCACGACGGACTGTACCAGGGAC |
| | | TCAGCACCGCCACCAAGGACACCTATGACGCTCTTCA |
| | | CATGCAGGCCCTGCCGCCTCGG |
| CD20-C5H4 | | |
| SEQ ID NO: | HCDR1 | SYNMH |
| 2043 (Kabat) | | |
| SEQ ID NO: | HCDR2 | AIYPGNGDTSYNPKFKG |
| 2044 (Kabat) | | |
| SEQ ID NO: | HCDR3 | SYFYGSSSWYFDV |
| 2045 (Kabat) | | |
| SEQ ID NO: | HCDR1 | GYTFTSY |
| 2046 (Chothia) | | |
| SEQ ID NO: | HCDR2 | YPGNGD |
| 2047 (Chothia) | | |
| SEQ ID NO: | HCDR3 | SYFYGSSSWYFDV |
| 2045 (Chothia) | | |
| SEQ ID NO: | HCDR1 | GYTFTSYN |
| 2048 (IMGT) | | |
| SEQ ID NO: | HCDR2 | TYPGNGDT |
| 2049 (IMGT) | | |
| SEQ ID NO: | HCDR3 | ARSYFYGSSSWYFDV |
| 2050 (IMGT) | | |
| SEQ ID NO: | HCDR1 | GYTFTSYNMH |
| 2051 (Combined | | |
| Chothia and | | |
| Kabat) | | |
| SEQ ID NO: | HCDR2 | AIYPGNGDTSYNPKFKG |
| 2044 (Combined | | |
| Chothia and | | |
| Kabat) | | |
| SEQ ID NO: | HCDR3 | SYFYGSSSWYFDV |
| 2045 (Combined | | |
| Chothia and | | |
| Kabat) | | |
| SEQ ID NO: | VH | QVQLVQSGAEVKKPGSSVKVSCKASGYTFTSYNMHWV |
| 2132 | | RQAPGQGLEWMGAIYPGNGDTSYNPKFKGRVTITADK |
| | | STRTAYMELSSLRSEDTAVYYCARSYFYGSSSWYFDV |
| | | WGQGTTVTVSS |
| SEQ ID NO: | DNA VH | CAAGTGCAACTCGTCCAGTCCGGTGCAGAAGTCAAG |
| 2139 | | AAGCCAGGTTCCTCGGTGAAAGTGTCCTGCAAAGCCT |
| | | CGGGTTACACCTTCACCTCGTACAATATGCACTGGGT |
| | | CCGCCAAGCTCCGGGACAAGGCCTGGAATGGATGGG |
| | | AGCGATCTACCCCGGAAACGGCGACACGTCCTACAA |
| | | CCCGAAGTTCAAGGGAAGAGTGACCATCACCGCCGA |
| | | CAAGTCCACCCGCACCGCGTACATGGAGCTTAGCAG |
| | | CCTGCGGAGCGAGGACACTGCCGTGTATTACTGCGCC |
| | | CGGTCCTACTTCTATGGATCATCCTCGTGGTACTTCG |
| | | ATGTCTGGGGCCAGGGGACCACCGTGACCGTGTCCA |
| | | GC |
| SEQ ID NO: | LCDR1 | RASSSVSSMH |
| 2054 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDRI | SSSVSS |
| 2056 (Chothia) | | |
| SEQ ID NO: | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVSS |
| 2036 (IMGT) | | |
| SEQ ID NO: | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVSSMH |
| 2054 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 (Combined | | |
| Chothia and | | |
| Kabat) | | |
| SEQ ID NO: | VL | DIQLTQSPSFLSASVGDRVTITCRASSSVSSMHWYQQKP |
| 2126 | | GKAPKPLIFATSNLASGVPSRFSGSGSGTEYTLTISSLQPE |
| | | DFATYYCQQWIFNPPTFGGGTKVEIK |
| SEQ ID NO: | DNA VL | GATATCCAGCTGACCCAGAGCCCTTCCTTCCTGTCCG |
| 2140 | | CTTCCGTGGGAGACAGAGTCACTATTACTTGTCGGGC |
| | | CTCCTCATCCGTGTCATCCATGCACTGGTACCAGCAG |
| | | AAGCCGGGAAAGGCCCCAAAGCCCTTGATCTTTGCC |
| | | ACTTCCAACCTGGCATCCGGCGTGCCCTCGAGGTTCT |
| | | CCGGGAGCGGTTCAGGGACCGAGTACACTCTGACCA |
| | | TTAGCAGCCTCCAGCCTGAGGACTTTGCCACCTACTA |
| | | CTGCCAGCAGTGGATTTTCAACCCGCCTACATTCGGA |
| | | GGGGGCACTAAGGTCGAAATCAAG |
| SEQ ID NO: | Linker | GGGGSGGGGSGGGGSGGGGS |
| 1010 | | |
| SEQ ID NO: | scFv (VH- | QVQLVQSGAEVKKPGSSVKVSCKASGYTFTSYNMHWV |
| 2141 | linker-VL) | RQAPGQGLEWMGAIYPGNGDTSYNPKFKGRVTITADK |
| | | STRTAYMELSSLRSEDTAVYYCARSYFYGSSSWYFDV |
| | | WGQGTTVTVSSGGGGSGGGGSGGGGSGGGGSDIQLTQ |
| | | SPSFLSASVGDRVTITCRASSSVSSMHWYQQKPGKAPKP |
| | | LIFATSNLASGVPSRFSGSGSGTEYTLTISSLQPEDFATY |
| | | YCQQWIFNPPTFGGGTKVEIK |
| SEQ ID NO: | DNA scFv | CAAGTGCAACTCGTCCAGTCCGGTGCAGAAGTCAAG |
| 2142 | (VH-linker- | AAGCCAGGTTCCTCGGTGAAAGTGTCCTGCAAAGCCT |
| | VL) | CGGGTTACACCTTCACCTCGTACAATATGCACTGGGT |
| | | CCGCCAAGCTCCGGGACAAGGCCTGGAATGGATGGG |
| | | AGCGATCTACCCCGGAAACGGCGACACGTCCTACAA |
| | | CCCGAAGTTCAAGGGAAGAGTGACCATCACCGCCGA |
| | | CAAGTCCACCCGCACCGCGTACATGGAGCTTAGCAG |
| | | CCTGCGGAGCGAGGACACTGCCGTGTATTACTGCGCC |
| | | CGGTCCTACTTCTATGGATCATCCTCGTGGTACTTCG |
| | | ATGTCTGGGGCCAGGGGACCACCGTGACCGTGTCCA |
| | | GCGGTGGCGGAGGCAGCGGCGGAGGAGGGTCTGGAG |
| | | GAGGCGGCTCGGGGGGAGGGGGCTCGGATATCCAGC |
| | | TGACCCAGAGCCCTTCCTTCCTGTCCGCTTCCGTGGG |
| | | AGACAGAGTCACTATTACTTGTCGGGCCTCCTCATCC |
| | | GTGTCATCCATGCACTGGTACCAGCAGAAGCCGGGA |
| | | AAGGCCCCAAAGCCCTTGATCTTTGCCACTTCCAACC |
| | | TGGCATCCGGCGTGCCCTCGAGGTTCTCCGGGAGCGG |
| | | TTCAGGGACCGAGTACACTCTGACCATTAGCAGCCTC |
| | | CAGCCTGAGGACTTTGCCACCTACTACTGCCAGCAGT |
| | | GGATTTTCAACCCGCCTACATTCGGAGGGGGCACTAA |
| | | GGTCGAAATCAAG |
| SEQ ID NO: | Full CAR | MALPVTALLLPLALLLHAARPQVQLVQSGAEVKKPGSS |
| 2143 | amino acid | VKVSCKASGYTFTSYNMHWVRQAPGQGLEWMGAIYP |
| | sequence | GNGDTSYNPKFKGRVTITADKSTRTAYMELSSLRSEDT |
| | | AVYYCARSYFYGSSSWYFDVWGQGTTVTVSSGGGGSG |
| | | GGGSGGGGSGGGGSDIQLTQSPSFLSASVGDRVTITCRA |
| | | SSSVSSMHWYQQKPGKAPKPLIFATSNLASGVPSRFSGS |
| | | GSGTEYTLTISSLQPEDFATYYCQQWIFNPPTFGGGTKV |
| | | EIKTTTPAPRPPTPAPTIASQPLSLRPEACRPAAGGAVHT |
| | | RGLDFACDIYIWAPLAGTCGVLLLSLVITLYCKRGRKKL |
| | | LYIFKQPFMRPVQTTQEEDGCSCRFPEEEEGGCELRVKF |
| | | SRSADAPAYQQGQNQLYNELNLGRREEYDVLDKRRGR |
| | | DPEMGGKPRRKNPQEGLYNELQKDKMAEAYSEIGMKG |
| | | ERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2144 | nucleic acid | CTCTTCTGCTCCACGCCGCTCGGCCCCAAGTGCAACT |
| | sequence | CGTCCAGTCCGGTGCAGAAGTCAAGAAGCCAGGTTC |
| | | CTCGGTGAAAGTGTCCTGCAAAGCCTCGGGTTACACC |
| | | TTCACCTCGTACAATATGCACTGGGTCCGCCAAGCTC |
| | | CGGGACAAGGCCTGGAATGGATGGGAGCGATCTACC |
| | | CCGGAAACGGCGACACGTCCTACAACCCGAAGTTCA |
| | | AGGGAAGAGTGACCATCACCGCCGACAAGTCCACCC |
| | | GCACCGCGTACATGGAGCTTAGCAGCCTGCGGAGCG |
| | | AGGACACTGCCGTGTATTACTGCGCCCGGTCCTACTT |
| | | CTATGGATCATCCTCGTGGTACTTCGATGTCTGGGGC |
| | | CAGGGGACCACCGTGACCGTGTCCAGCGGTGGCGGA |
| | | GGCAGCGGCGGAGGAGGGTCTGGAGGAGGCGGCTCG |
| | | GGGGGAGGGGGCTCGGATATCCAGCTGACCCAGAGC |
| | | CCTTCCTTCCTGTCCGCTTCCGTGGGAGACAGAGTCA |
| | | CTATTACTTGTCGGGCCTCCTCATCCGTGTCATCCATG |
| | | CACTGGTACCAGCAGAAGCCGGGAAAGGCCCCAAAG |
| | | CCCTTGATCTTTGCCACTTCCAACCTGGCATCCGGCG |
| | | TGCCCTCGAGGTTCTCCGGGAGCGGTTCAGGGACCGA |
| | | GTACACTCTGACCATTAGCAGCCTCCAGCCTGAGGAC |
| | | TTTGCCACCTACTACTGCCAGCAGTGGATTTTCAACC |
| | | CGCCTACATTCGGAGGGGGCACTAAGGTCGAAATCA |
| | | AGACCACTACCCCAGCACCGAGGCCACCCACCCCGG |
| | | CTCCTACCATCGCCTCCCAGCCTCTGTCCCTGCGTCCG |
| | | GAGGCATGTAGACCCGCAGCTGGTGGGGCCGTGCAT |
| | | ACCCGGGGTCTTGACTTCGCCTGCGATATCTACATTT |
| | | GGGCCCCTCTGGCTGGTACTTGCGGGGTCCTGCTGCT |
| | | TTCACTCGTGATCACTCTTTACTGTAAGCGCGGTCGG |
| | | AAGAAGCTGCTGTACATCTTTAAGCAACCCTTCATGA |
| | | GGCCTGTGCAGACTACTCAAGAGGAGGACGGCTGTT |
| | | CATGCCGGTTCCCAGAGGAGGAGGAAGGCGGCTGCG |
| | | AACTGCGCGTGAAATTCAGCCGCAGCGCAGATGCTC |
| | | CAGCCTACCAGCAGGGGCAGAACCAGCTCTACAACG |
| | | AACTCAATCTTGGTCGGAGAGAGGAGTACGACGTGC |
| | | TGGACAAGCGGAGAGGACGGGACCCAGAAATGGGC |
| | | GGGAAGCCGCGCAGAAAGAATCCCCAAGAGGGCCTG |
| | | TACAACGAGCTCCAAAAGGATAAGATGGCAGAAGCC |
| | | TATAGCGAGATTGGTATGAAAGGGGAACGCAGAAGA |
| | | GGCAAAGGCCACGACGGACTGTACCAGGGACTCAGC |
| | | ACCGCCACCAAGGACACCTATGACGCTCTTCACATGC |
| | | AGGCCCTGCCGCCTCGG |
| CD20-C8H1 | | |
| SEQ ID NO: | HCDR1 | RYNMH |
| 2145 (Kabat) | | |
| SEQ ID NO: | HCDR2 | AIYPGNGDTSYSQKFKG |
| 2146 (Kabat) | | |
| SEQ ID NO: | HCDR3 | SFFYGSSDWYFDV |
| 2147 (Kabat) | | |
| SEQ ID NO: | HCDR1 | GYTFTRY |
| 2148 (Chothia) | | |
| SEQ ID NO: | HCDR2 | YPGNGD |
| 2047 (Chothia) | | |
| SEQ ID NO: | HCDR3 | SFFYGSSDWYFDV |
| 2147 (Chothia) | | |
| SEQ ID NO: | HCDR1 | GYTFTRYN |
| 2149 (IMGT) | | |
| SEQ ID NO: | HCDR2 | IYPGNGDT |
| 2049 (IMGT) | | |
| SEQ ID NO: | HCDR3 | ARSFFYGSSDWYFDV |
| 2150 (IMGT) | | |
| SEQ ID NO: | HCDR1 | GYTFTRYNMH |
| 2151 (Combined | | |
| Chothia and | | |
| Kabat | | |
| SEQ ID NO: | HCDR2 | AIYPGNGDTSYSQKFKG |
| 2146 (Combined | | |
| Chothia and | | |
| Kabat | | |
| SEQ ID NO: | HCDR3 | SFFYGSSDWYFDV |
| 2147 (Combined | | |
| Chothia and | | |
| Kabat) | | |
| SEQ ID NO: | VH | QVQLVQSGAEVKKPGASVKVSCKASGYTFTRYNMHW |
| 2152 | | VRQAPGQRLEWMGAIYPGNGDTSYSQKFKGRVTITAD |
| | | KSASTAYMELSSLRSEDTAVYYCARSFFYGSSDWYFDV |
| | | WGQGTTVTVSS |
| SEQ ID NO: | DNA VH | CAAGTCCAACTCGTCCAGTCAGGAGCAGAAGTCAAG |
| 2153 | | AAACCAGGAGCATCCGTGAAAGTGTCGTGCAAAGCC |
| | | TCTGGCTACACCTTCACCCGGTACAACATGCACTGGG |
| | | TCAGACAGGCCCCGGGACAGCGGCTCGAGTGGATGG |
| | | GTGCCATCTACCCCGGCAACGGGGACACCTCCTACTC |
| | | CCAAAAGTTCAAGGGTCGCGTGACCATCACGGCGGA |
| | | TAAGTCGGCCAGCACTGCGTACATGGAATTGTCATCC |
| | | CTGCGCTCCGAGGATACCGCCGTGTATTACTGCGCGC |
| | | GGTCCTTCTTCTACGGCTCCTCCGATTGGTACTTCGAC |
| | | GTCTGGGGACAGGGAACTACCGTGACCGTGTCCTCC |
| SEQ ID NO: | LCDR1 | RASSSVNNMH |
| 2154 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDR1 | SSSVNN |
| 2155 (Chothia) | | |
| SEQ ID NO: | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVNN |
| 2156 (IMGT) | | |
| SEQ ID NO: | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVNNMH |
| 2154 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 (Combined | | |
| Chothia and | | |
| Kabat | | |
| SEQ ID NO: | VL | EIVLTQSPDFQSVTPKEKVTITCRASSSVNNMHWYQQK |
| 2157 | | PDQSPKPLIYATSNLASGVPSRFSGSGSGTDYTLTINSLE |
| | | AEDAATYYCQQWIFNPPTFGQGTKLEIK |
| SEQ ID NO: | DNA VL | GAAATCGTGCTGACTCAGTCGCCGGACTTCCAAAGCG |
| 2158 | | TGACCCCAAAGGAGAAGGTCACCATCACCTGTAGAG |
| | | CCTCATCGTCCGTGAACAATATGCACTGGTACCAGCA |
| | | GAAGCCGGACCAGTCCCCTAAGCCCCTGATCTACGCC |
| | | ACTTCCAACCTGGCCTCCGGCGTGCCGTCGAGGTTCA |
| | | GCGGCTCGGGCAGCGGGACCGACTACACCCTGACCA |
| | | TCAACAGCCTTGAAGCTGAGGACGCCGCTACCTACTA |
| | | CTGCCAGCAGTGGATTTTCAACCCTCCCACATTTGGA |
| | | CAGGGCACTAAGCTGGAGATTAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTRYNMHW |
| 2159 | linker-VL) | VRQAPGQRLEWMGAIYPGNGDTSYSQKFKGRVTITAD |
| | | KSASTAYMELSSLRSEDTAVYYCARSFFYGSSDWYFDV |
| | | WGQGTTVTVSSGGGGSGGGGSGGGGSGGGGSEIVLTQ |
| | | SPDFQSVTPKEKVTITCRASSSVNNMHWYQQKPDQSPK |
| | | PLIYATSNLASGVPSRFSGSGSGTDYTLTINSLEAEDAAT |
| | | YYCQQWIFNPPTFGQGTKLEIK |
| SEQ ID NO: | DNA scFv | CAAGTCCAACTCGTCCAGTCAGGAGCAGAAGTCAAG |
| 2160 | (VH-linker- | AAACCAGGAGCATCCGTGAAAGTGTCGTGCAAAGCC |
| | VL | TCTGGCTACACCTTCACCCGGTACAACATGCACTGGG |
| | | TCAGACAGGCCCCGGGACAGCGGCTCGAGTGGATGG |
| | | GTGCCATCTACCCCGGCAACGGGGACACCTCCTACTC |
| | | CCAAAAGTTCAAGGGTCGCGTGACCATCACGGCGGA |
| | | TAAGTCGGCCAGCACTGCGTACATGGAATTGTCATCC |
| | | CTGCGCTCCGAGGATACCGCCGTGTATTACTGCGCGC |
| | | GGTCCTTCTTCTACGGCTCCTCCGATTGGTACTTCGAC |
| | | GTCTGGGGACAGGGAACTACCGTGACCGTGTCCTCCG |
| | | GGGGTGGCGGGAGCGGAGGGGGCGGAAGCGGGGGT |
| | | GGAGGATCAGGAGGCGGAGGCTCCGAAATCGTGCTG |
| | | ACTCAGTCGCCGGACTTCCAAAGCGTGACCCCAAAG |
| | | GAGAAGGTCACCATCACCTGTAGAGCCTCATCGTCCG |
| | | TGAACAATATGCACTGGTACCAGCAGAAGCCGGACC |
| | | AGTCCCCTAAGCCCCTGATCTACGCCACTTCCAACCT |
| | | GGCCTCCGGCGTGCCGTCGAGGTTCAGCGGCTCGGGC |
| | | AGCGGGACCGACTACACCCTGACCATCAACAGCCTT |
| | | GAAGCTGAGGACGCCGCTACCTACTACTGCCAGCAG |
| | | TGGATTTTCAACCCTCCCACATTTGGACAGGGCACTA |
| | | AGCTGGAGATTAAG |
| SEQ ID NO: | Full CAR | MALPVTALLLPLALLLHAARPQVQLVQSGAEVKKPGAS |
| 2161 | amino acid | VKVSCKASGYTFTRYNMHWVRQAPGQRLEWMGAIYP |
| | sequence | GNGDTSYSQKFKGRVTITADKSASTAYMELSSLRSEDT |
| | | AVYYCARSFFYGSSDWYFDVWGQGTTVTVSSGGGGSG |
| | | GGGSGGGGSGGGGSEIVLTQSPDFQSVTPKEKVTITCRA |
| | | SSSVNNMHWYQQKPDQSPKPLIYATSNLASGVPSRFSG |
| | | SGSGTDYTLTINSLEAEDAATYYCQQWIFNPPTFGQGTK |
| | | LEIKTTTPAPRPPTPAPTIASQPLSLRPEACRPAAGGAVH |
| | | TRGLDFACDIYIWAPLAGTCGVLLLSLVITLYCKRGRKK |
| | | LLYIFKQPFMRPVQTTQEEDGCSCRFPEEEEGGCELRVK |
| | | FSRSADAPAYQQGQNQLYNELNLGRREEYDVLDKRRG |
| | | RDPEMGGKPRRKNPQEGLYNELQKDKMAEAYSEIGMK |
| | | GERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2162 | nucleic acid | CTCTTCTGCTCCACGCCGCTCGGCCCCAAGTCCAACT |
| | sequence | CGTCCAGTCAGGAGCAGAAGTCAAGAAACCAGGAGC |
| | | ATCCGTGAAAGTGTCGTGCAAAGCCTCTGGCTACACC |
| | | TTCACCCGGTACAACATGCACTGGGTCAGACAGGCCC |
| | | CGGGACAGCGGCTCGAGTGGATGGGTGCCATCTACC |
| | | CCGGCAACGGGGACACCTCCTACTCCCAAAAGTTCA |
| | | AGGGTCGCGTGACCATCACGGCGGATAAGTCGGCCA |
| | | GCACTGCGTACATGGAATTGTCATCCCTGCGCTCCGA |
| | | GGATACCGCCGTGTATTACTGCGCGCGGTCCTTCTTC |
| | | TACGGCTCCTCCGATTGGTACTTCGACGTCTGGGGAC |
| | | AGGGAACTACCGTGACCGTGTCCTCCGGGGGTGGCG |
| | | GGAGCGGAGGGGGCGGAAGCGGGGGTGGAGGATCA |
| | | GGAGGCGGAGGCTCCGAAATCGTGCTGACTCAGTCG |
| | | CCGGACTTCCAAAGCGTGACCCCAAAGGAGAAGGTC |
| | | ACCATCACCTGTAGAGCCTCATCGTCCGTGAACAATA |
| | | TGCACTGGTACCAGCAGAAGCCGGACCAGTCCCCTA |
| | | AGCCCCTGATCTACGCCACTTCCAACCTGGCCTCCGG |
| | | CGTGCCGTCGAGGTTCAGCGGCTCGGGCAGCGGGAC |
| | | CGACTACACCCTGACCATCAACAGCCTTGAAGCTGAG |
| | | GACGCCGCTACCTACTACTGCCAGCAGTGGATTTTCA |
| | | ACCCTCCCACATTTGGACAGGGCACTAAGCTGGAGAT |
| | | TAAGACCACTACCCCAGCACCGAGGCCACCCACCCC |
| | | GGCTCCTACCATCGCCTCCCAGCCTCTGTCCCTGCGT |
| | | CCGGAGGCATGTAGACCCGCAGCTGGTGGGGCCGTG |
| | | CATACCCGGGGTCTTGACTTCGCCTGCGATATCTACA |
| | | TTTGGGCCCCTCTGGCTGGTACTTGCGGGGTCCTGCT |
| | | GCTTTCACTCGTGATCACTCTTTACTGTAAGCGCGGT |
| | | CGGAAGAAGCTGCTGTACATCTTTAAGCAACCCTTCA |
| | | TGAGGCCTGTGCAGACTACTCAAGAGGAGGACGGCT |
| | | GTTCATGCCGGTTCCCAGAGGAGGAGGAAGGCGGCT |
| | | GCGAACTGCGCGTGAAATTCAGCCGCAGCGCAGATG |
| | | CTCCAGCCTACCAGCAGGGGCAGAACCAGCTCTACA |
| | | ACGAACTCAATCTTGGTCGGAGAGAGGAGTACGACG |
| | | TGCTGGACAAGCGGAGAGGACGGGACCCAGAAATGG |
| | | GCGGGAAGCCGCGCAGAAAGAATCCCCAAGAGGGCC |
| | | TGTACAACGAGCTCCAAAAGGATAAGATGGCAGAAG |
| | | CCTATAGCGAGATTGGTATGAAAGGGGAACGCAGAA |
| | | GAGGCAAAGGCCACGACGGACTGTACCAGGGACTCA |
| | | GCACCGCCACCAAGGACACCTATGACGCTCTTCACAT |
| | | GCAGGCCCTGCCGCCTCGG |
| CD20-C8H2 | | |
| SEQ ID NO: | HCDR1 | RYNMH |
| 2145 (Kabat) | | |
| SEQ ID NO: | HCDR2 | AIYPGNGDTSYSQKFKG |
| 2146 (Kabat) | | |
| SEQ ID NO: | HCDR3 | SFFYGSSDWYFDV |
| 2147 (Kabat) | | |
| SEQ ID NO: | HCDR1 | GYTFTRY |
| 2148 (Chothia) | | |
| SEQ ID NO: | HCDR2 | YPGNGD |
| 2047 (Chothia) | | |
| SEQ ID NO: | HCDR3 | SFFYGSSDWYFDV |
| 2147 (Chothia) | | |
| SEQ ID NO: | HCDR1 | GYTFTRYN |
| 2149 (IMGT) | | |
| SEQ ID NO: | HCDR2 | IYPGNGDT |
| 2049 (IMGT) | | |
| SEQ ID NO: | HCDR3 | ARSFFYGSSDWYFDV |
| 2150 (IMGT) | | |
| SEQ ID NO: | HCDR1 | GYTFTRYNMH |
| 2151 (Combined | | |
| Chothia and | | |
| Kabat) | | |
| SEQ ID NO: | HCDR2 | AIYPGNGDTSYSQKFKG |
| 2146 (Combined | | |
| Chothia and | | |
| Kabat) | | |
| SEQ ID NO: | HCDR3 | SFFYGSSDWYFDV |
| 2147 (Combined | | |
| Chothia and | | |
| Kabat) | | |
| SEQ ID NO: | VH | QVQLVQSGAEVKKPGASVKVSCKASGYTFTRYNMHW |
| 2152 | | VRQAPGQRLEWMGAIYPGNGDTSYSQKFKGRVTITAD |
| | | KSASTAYMELSSLRSEDTAVYYCARSFFYGSSDWYFDV |
| | | WGQGTTVTVSS |
| SEQ ID NO: | DNA VH | CAAGTGCAACTCGTCCAATCCGGCGCGGAAGTCAAA |
| 2163 | | AAGCCTGGAGCCTCCGTCAAAGTGTCCTGCAAGGCCT |
| | | CCGGTTACACTTTCACTCGCTACAACATGCATTGGGT |
| | | GCGGCAGGCCCCGGGACAGCGCCTGGAATGGATGGG |
| | | CGCAATCTACCCCGGCAACGGAGACACCTCCTATTCC |
| | | CAAAAGTTCAAGGGAAGGGTCACAATCACGGCCGAC |
| | | AAGAGCGCCTCAACTGCCTACATGGAGCTGAGCAGC |
| | | CTCAGATCCGAAGATACCGCGGTGTACTACTGCGCCC |
| | | GGAGCTTCTTCTACGGTTCGTCTGATTGGTACTTTGAC |
| | | GTCTGGGGCCAGGGAACCACCGTGACCGTGTCGTCC |
| SEQ ID NO: | LCDR1 | RASSSVNNMH |
| 2154 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDR1 | SSSVNN |
| 2155 (Chothia) | | |
| SEQ ID NO: | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVNN |
| 2156 (IMGT) | | |
| SEQ ID NO: | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVNNMH |
| 2154 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 (Combined | | |
| Chothia and | | |
| Kabat) | | |
| SEQ ID NO: | VL | DIQLTQSPSFLSASVGDRVTITCRASSSVNNMHWYQQK |
| 2164 | | PGKAPKPLIYATSNLASGVPSRFSGSGSGTEYTLTISSLQ |
| | | PEDFATYYCQQWIFNPPTFGQGTKLEIK |
| SEQ ID NO: | DNA VL | GACATCCAGCTTACCCAGTCGCCATCATTCCTGTCCG |
| 2165 | | CATCAGTGGGTGATCGCGTGACCATTACCTGTCGGGC |
| | | GTCCTCCTCCGTGAACAACATGCACTGGTACCAGCAG |
| | | AAGCCGGGGAAGGCTCCCAAGCCTCTGATCTACGCC |
| | | ACTAGCAATTTGGCCAGCGGCGTGCCTTCGAGATTCT |
| | | CGGGGTCGGGCTCAGGAACCGAGTATACCCTGACCA |
| | | TTTCCTCCCTCCAACCGGAGGACTTTGCTACTTACTAC |
| | | TGCCAGCAGTGGATTTTCAACCCCCCGACTTTCGGAC |
| | | AGGGCACCAAGCTGGAAATCAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGYTFTRYNMHW |
| 2166 | linker-VL) | VRQAPGQRLEWMGAIYPGNGDTSYSQKFKGRVTITAD |
| | | KSASTAYMELSSLRSEDTAVYYCARSFFYGSSDWYFDV |
| | | WGQGTTVTVSSGGGGSGGGGSGGGGSGGGGSDIQLTQ |
| | | SPSFLSASVGDRVTITCRASSSVNNMHWYQQKPGKAPK |
| | | PLIYATSNLASGVPSRFSGSGSGTEYTLTISSLQPEDFAT |
| | | YYCQQWIFNPPTFGQGTKLEIK |
| SEQ ID NO: | DNA scFv | CAAGTGCAACTCGTCCAATCCGGCGCGGAAGTCAAA |
| 2167 | (VH-linker- | AAGCCTGGAGCCTCCGTCAAAGTGTCCTGCAAGGCCT |
| | VL) | CCGGTTACACTTTCACTCGCTACAACATGCATTGGGT |
| | | GCGGCAGGCCCCGGGACAGCGCCTGGAATGGATGGG |
| | | CGCAATCTACCCCGGCAACGGAGACACCTCCTATTCC |
| | | CAAAAGTTCAAGGGAAGGGTCACAATCACGGCCGAC |
| | | AAGAGCGCCTCAACTGCCTACATGGAGCTGAGCAGC |
| | | CTCAGATCCGAAGATACCGCGGTGTACTACTGCGCCC |
| | | GGAGCTTCTTCTACGGTTCGTCTGATTGGTACTTTGAC |
| | | GTCTGGGGCCAGGGAACCACCGTGACCGTGTCGTCC |
| | | GGTGGCGGAGGGAGCGGTGGAGGAGGCTCCGGGGG |
| | | AGGAGGCAGCGGCGGGGGAGGCAGCGACATCCAGCT |
| | | TACCCAGTCGCCATCATTCCTGTCCGCATCAGTGGGT |
| | | GATCGCGTGACCATTACCTGTCGGGCGTCCTCCTCCG |
| | | TGAACAACATGCACTGGTACCAGCAGAAGCCGGGGA |
| | | AGGCTCCCAAGCCTCTGATCTACGCCACTAGCAATTT |
| | | GGCCAGCGGCGTGCCTTCGAGATTCTCGGGGTCGGGC |
| | | TCAGGAACCGAGTATACCCTGACCATTICCTCCCTCC |
| | | AACCGGAGGACTTTGCTACTTACTACTGCCAGCAGTG |
| | | GATTTTCAACCCCCCGACTTTCGGACAGGGCACCAAG |
| | | CTGGAAATCAAG |
| SEQ ID NO: | Full CAR | MALPVTALLLPLALLLHAARPQVQLVQSGAEVKKPGAS |
| 2168 | amino acid | VKVSCKASGYTFTRYNMHWVRQAPGQRLEWMGAIYP |
| | sequence | GNGDTSYSQKFKGRVTITADKSASTAYMELSSLRSEDT |
| | | AVYYCARSFFYGSSDWYFDVWGQGTTVTVSSGGGGSG |
| | | GGGSGGGGSGGGGSDIQLTQSPSFLSASVGDRVTITCRA |
| | | SSSVNNMHWYQQKPGKAPKPLIYATSNLASGVPSRFSG |
| | | SGSGTEYTLTISSLQPEDFATYYCQQWIFNPPTFGQGTK |
| | | LEIKTTTPAPRPPTPAPTIASQPLSLRPEACRPAAGGAVH |
| | | TRGLDFACDIYIWAPLAGTCGVLLLSLVITLYCKRGRKK |
| | | LLYIFKQPFMRPVQTTQEEDGCSCRFPEEEEGGCELRVK |
| | | FSRSADAPAYQQGQNQLYNELNLGRREEYDVLDKRRG |
| | | RDPEMGGKPRRKNPQEGLYNELQKDKMAEAYSEIGMK |
| | | GERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2169 | nucleic acid | CTCTTCTGCTCCACGCCGCTCGGCCCCAAGTGCAACT |
| | sequence | CGTCCAATCCGGCGCGGAAGTCAAAAAGCCTGGAGC |
| | | CTCCGTCAAAGTGTCCTGCAAGGCCTCCGGTTACACT |
| | | TTCACTCGCTACAACATGCATTGGGTGCGGCAGGCCC |
| | | CGGGACAGCGCCTGGAATGGATGGGCGCAATCTACC |
| | | CCGGCAACGGAGACACCTCCTATTCCCAAAAGTTCAA |
| | | GGGAAGGGTCACAATCACGGCCGACAAGAGCGCCTC |
| | | AACTGCCTACATGGAGCTGAGCAGCCTCAGATCCGA |
| | | AGATACCGCGGTGTACTACTGCGCCCGGAGCTTCTTC |
| | | TACGGTTCGTCTGATTGGTACTTTGACGTCTGGGGCC |
| | | AGGGAACCACCGTGACCGTGTCGTCCGGTGGCGGAG |
| | | GGAGCGGTGGAGGAGGCTCCGGGGGAGGAGGCAGC |
| | | GGCGGGGGAGGCAGCGACATCCAGCTTACCCAGTCG |
| | | CCATCATTCCTGTCCGCATCAGTGGGTGATCGCGTGA |
| | | CCATTACCTGTCGGGCGTCCTCCTCCGTGAACAACAT |
| | | GCACTGGTACCAGCAGAAGCCGGGGAAGGCTCCCAA |
| | | GCCTCTGATCTACGCCACTAGCAATTTGGCCAGCGGC |
| | | GTGCCTTCGAGATTCTCGGGGTCGGGCTCAGGAACCG |
| | | AGTATACCCTGACCATTTCCTCCCTCCAACCGGAGGA |
| | | CTTTGCTACTTACTACTGCCAGCAGTGGATTTTCAAC |
| | | CCCCCGACTTTCGGACAGGGCACCAAGCTGGAAATC |
| | | AAGACCACTACCCCAGCACCGAGGCCACCCACCCCG |
| | | GCTCCTACCATCGCCTCCCAGCCTCTGTCCCTGCGTCC |
| | | GGAGGCATGTAGACCCGCAGCTGGTGGGGCCGTGCA |
| | | TACCCGGGGTCTTGACTTCGCCTGCGATATCTACATT |
| | | TGGGCCCCTCTGGCTGGTACTTGCGGGGTCCTGCTGC |
| | | TTTCACTCGTGATCACTCTTTACTGTAAGCGCGGTCG |
| | | GAAGAAGCTGCTGTACATCTTTAAGCAACCCTTCATG |
| | | AGGCCTGTGCAGACTACTCAAGAGGAGGACGGCTGT |
| | | TCATGCCGGTTCCCAGAGGAGGAGGAAGGCGGCTGC |
| | | GAACTGCGCGTGAAATTCAGCCGCAGCGCAGATGCT |
| | | CCAGCCTACCAGCAGGGGCAGAACCAGCTCTACAAC |
| | | GAACTCAATCTTGGTCGGAGAGAGGAGTACGACGTG |
| | | CTGGACAAGCGGAGAGGACGGGACCCAGAAATGGGC |
| | | GGGAAGCCGCGCAGAAAGAATCCCCAAGAGGGCCTG |
| | | TACAACGAGCTCCAAAAGGATAAGATGGCAGAAGCC |
| | | TATAGCGAGATTGGTATGAAAGGGGAACGCAGAAGA |
| | | GGCAAAGGCCACGACGGACTGTACCAGGGACTCAGC |
| | | ACCGCCACCAAGGACACCTATGACGCTCTTCACATGC |
| | | AGGCCCTGCCGCCTCGG |
| CD20-C8H3 | | |
| SEQ ID NO: | HCDR1 | RYNMH |
| 2145 (Kabat) | | |
| SEQ ID NO: | HCDR2 | AIYPGNGDTSYSQKFKG |
| 2146 (Kabat) | | |
| SEQ ID NO: | HCDR3 | SFFYGSSDWYFDV |
| 2147 (Kabat) | | |
| SEQ ID NO: | HCDR1 | GYTFTRY |
| 2148 (Chothia) | | |
| SEQ ID NO: | HCDR2 | YPGNGD |
| 2047 (Chothia) | | |
| SEQ ID NO: | HCDR3 | SFFYGSSDWYFDV |
| 2147 (Chothia) | | |
| SEQ ID NO: | HCDR1 | GYTFTRYN |
| 2149 (IMGT) | | |
| SEQ ID NO: | HCDR2 | IYPGNGDT |
| 2049 (IMGT) | | |
| SEQ ID NO: | HCDR3 | ARSFFYGSSDWYFDV |
| 2150 (IMGT) | | |
| SEQ ID NO: | HCDR | GYTFTRYNMH |
| 2151 (Combined | | |
| Chothia and | | |
| Kabat | | |
| SEQ ID NO: | HCDR2 | AIYPGNGDTSYSQKFKG |
| 2146 (Combined | | |
| Chothia and | | |
| Kabat | | |
| SEQ ID NO: | HCDR3 | SFFYGSSDWYFDV |
| 2147 (Combined | | |
| Chothia and | | |
| Kabat | | |
| SEQ ID NO: | VH | QVQLVQSGAEVKKPGSSVKVSCKASGYTFTRYNMHW |
| 2170 | | VRQAPGQGLEWMGAIYPGNGDTSYSQKFKGRVTITAD |
| | | KSTSTAYMELSSLRSEDTAVYYCARSFFYGSSDWYFDV |
| | | WGQGTTVTVSS |
| SEQ ID NO: | DNA VH | CAAGTGCAACTCGTCCAGTCCGGTGCAGAAGTCAAG |
| 2171 | | AAGCCTGGTTCCTCCGTGAAAGTGTCCTGCAAAGCGT |
| | | CTGGCTACACCTTCACCCGGTACAATATGCACTGGGT |
| | | CAGACAGGCGCCCGGACAGGGCCTGGAGTGGATGGG |
| | | GGCCATCTACCCTGGGAACGGCGACACTAGCTACTCC |
| | | CAAAAGTTCAAGGGCCGCGTGACGATTACCGCCGAC |
| | | AAGTCAACCAGCACTGCCTATATGGAGCTGAGCTCGC |
| | | TTCGGAGCGAAGATACCGCCGTGTACTACTGCGCTCG |
| | | GAGCTTCTTCTACGGGTCCTCGGATTGGTACTTCGAC |
| | | GTCTGGGGCCAGGGGACTACTGTGACCGTGTCCTCC |
| SEQ ID NO: | LCDR1 | RASSSVNNMH |
| 2154 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDR1 | SSSVNN |
| 2155 (Chothia) | | |
| SEQ ID NO: | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVNN |
| 2156 (IMGT) | | |
| SEQ ID NO: | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVNNMH |
| 2154 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 (Combined | | |
| Chothia and | | |
| Kabat) | | |
| SEQ ID NO: | VL | EIVLTQSPDFQSVTPKEKVTITCRASSSVNNMHWYQQK |
| 2157 | | PDQSPKPLIYATSNLASGVPSRFSGSGSGTDYTLTINSLE |
| | | AEDAATYYCQQWIFNPPTFGQGTKLEIK |
| SEQ ID NO: | DNA VL | GAAATCGTGCTGACCCAGTCCCCGGACTTTCAGTCAG |
| 2172 | | TGACTCCCAAGGAGAAGGTCACCATTACTTGTCGCGC |
| | | CTCCTCCTCGGTGAACAACATGCACTGGTACCAGCAG |
| | | AAGCCGGACCAGTCCCCGAAGCCCCTGATCTATGCTA |
| | | CCTCCAACTTGGCGTCCGGCGTGCCGTCAAGGTTCAG |
| | | CGGATCGGGTTCCGGGACAGACTACACCCTGACTATT |
| | | AACTCACTCGAGGCCGAGGATGCCGCCACCTACTACT |
| | | GCCAGCAGTGGATCTTCAACCCTCCAACCTTCGGACA |
| | | AGGAACCAAGCTGGAAATCAAG |
| SEQ ID NO: | Linker | GGGGSGGGGSGGGGSGGGGS |
| 1010 | | |
| SEQ ID NO: | scFv (VH- | QVQLVQSGAEVKKPGSSVKVSCKASGYTFTRYNMHW |
| 2173 | linker-VL) | VRQAPGQGLEWMGAIYPGNGDTSYSQKFKGRVTITAD |
| | | KSTSTAYMELSSLRSEDTAVYYCARSFFYGSSDWYFDV |
| | | WGQGTTVTVSSGGGGSGGGGSGGGGSGGGGSEIVLTQ |
| | | SPDFQSVTPKEKVTITCRASSSVNNMHWYQQKPDQSPK |
| | | PLIYATSNLASGVPSRFSGSGSGTDYTLTINSLEAEDAAT |
| | | YYCQQWIFNPPTFGQGTKLEIK |
| SEQ ID NO: | DNA scFv | CAAGTGCAACTCGTCCAGTCCGGTGCAGAAGTCAAG |
| 2174 | (VH-linker- | AAGCCTGGTTCCTCCGTGAAAGTGTCCTGCAAAGCGT |
| | VL) | CTGGCTACACCTTCACCCGGTACAATATGCACTGGGT |
| | | CAGACAGGCGCCCGGACAGGGCCTGGAGTGGATGGG |
| | | GGCCATCTACCCTGGGAACGGCGACACTAGCTACTCC |
| | | CAAAAGTTCAAGGGCCGCGTGACGATTACCGCCGAC |
| | | AAGTCAACCAGCACTGCCTATATGGAGCTGAGCTCGC |
| | | TTCGGAGCGAAGATACCGCCGTGTACTACTGCGCTCG |
| | | GAGCTTCTTCTACGGGTCCTCGGATTGGTACTTCGAC |
| | | GTCTGGGGCCAGGGGACTACTGTGACCGTGTCCTCCG |
| | | GGGGAGGAGGATCGGGCGGAGGCGGTTCGGGAGGC |
| | | GGCGGAAGCGGAGGCGGAGGTTCAGAAATCGTGCTG |
| | | ACCCAGTCCCCGGACTTTCAGTCAGTGACTCCCAAGG |
| | | AGAAGGTCACCATTACTTGTCGCGCCTCCTCCTCGGT |
| | | GAACAACATGCACTGGTACCAGCAGAAGCCGGACCA |
| | | GTCCCCGAAGCCCCTGATCTATGCTACCTCCAACTTG |
| | | GCGTCCGGCGTGCCGTCAAGGTTCAGCGGATCGGGTT |
| | | CCGGGACAGACTACACCCTGACTATTAACTCACTCGA |
| | | GGCCGAGGATGCCGCCACCTACTACTGCCAGCAGTG |
| | | GATCTTCAACCCTCCAACCTTCGGACAAGGAACCAAG |
| | | CTGGAAATCAAG |
| SEQ ID NO: | Full CAR | MALPVTALLLPLALLLHAARPQVQLVQSGAEVKKPGSS |
| 2175 | amino acid | VKVSCKASGYTFTRYNMHWVRQAPGQGLEWMGAIYP |
| | sequence | GNGDTSYSQKFKGRVTITADKSTSTAYMELSSLRSEDT |
| | | AVYYCARSFFYGSSDWYFDVWGQGTTVTVSSGGGGSG |
| | | GGGSGGGGSGGGGSEIVLTQSPDFQSVTPKEKVTITCRA |
| | | SSSVNNMHWYQQKPDQSPKPLIYATSNLASGVPSRFSG |
| | | SGSGTDYTLTINSLEAEDAATYYCQQWIFNPPTFGQGTK |
| | | LEIKTTTPAPRPPTPAPTIASQPLSLRPEACRPAAGGAVH |
| | | TRGLDFACDIYIWAPLAGTCGVLLLSLVITLYCKRGRKK |
| | | LLYIFKQPFMRPVQTTQEEDGCSCRFPEEEEGGCELRVK |
| | | FSRSADAPAYQQGQNQLYNELNLGRREEYDVLDKRRG |
| | | RDPEMGGKPRRKNPQEGLYNELQKDKMAEAYSEIGMK |
| | | GERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2176 | nucleic acid | CTCTTCTGCTCCACGCCGCTCGGCCCCAAGTGCAACT |
| | sequence | CGTCCAGTCCGGTGCAGAAGTCAAGAAGCCTGGTTCC |
| | | TCCGTGAAAGTGTCCTGCAAAGCGTCTGGCTACACCT |
| | | TCACCCGGTACAATATGCACTGGGTCAGACAGGCGC |
| | | CCGGACAGGGCCTGGAGTGGATGGGGGCCATCTACC |
| | | CTGGGAACGGCGACACTAGCTACTCCCAAAAGTTCA |
| | | AGGGCCGCGTGACGATTACCGCCGACAAGTCAACCA |
| | | GCACTGCCTATATGGAGCTGAGCTCGCTTCGGAGCGA |
| | | AGATACCGCCGTGTACTACTGCGCTCGGAGCTTCTTC |
| | | TACGGGTCCTCGGATTGGTACTTCGACGTCTGGGGCC |
| | | AGGGGACTACTGTGACCGTGTCCTCCGGGGGAGGAG |
| | | GATCGGGCGGAGGCGGTTCGGGAGGCGGCGGAAGCG |
| | | GAGGCGGAGGTTCAGAAATCGTGCTGACCCAGTCCC |
| | | CGGACTTTCAGTCAGTGACTCCCAAGGAGAAGGTCA |
| | | CCATTACTTGTCGCGCCTCCTCCTCGGTGAACAACAT |
| | | GCACTGGTACCAGCAGAAGCCGGACCAGTCCCCGAA |
| | | GCCCCTGATCTATGCTACCTCCAACTTGGCGTCCGGC |
| | | GTGCCGTCAAGGTTCAGCGGATCGGGTTCCGGGACA |
| | | GACTACACCCTGACTATTAACTCACTCGAGGCCGAGG |
| | | ATGCCGCCACCTACTACTGCCAGCAGTGGATCTTCAA |
| | | CCCTCCAACCTTCGGACAAGGAACCAAGCTGGAAAT |
| | | CAAGACCACTACCCCAGCACCGAGGCCACCCACCCC |
| | | GGCTCCTACCATCGCCTCCCAGCCTCTGTCCCTGCGT |
| | | CCGGAGGCATGTAGACCCGCAGCTGGTGGGGCCGTG |
| | | CATACCCGGGGTCTTGACTTCGCCTGCGATATCTACA |
| | | TTTGGGCCCCTCTGGCTGGTACTTGCGGGGTCCTGCT |
| | | GCTTTCACTCGTGATCACTCTTTACTGTAAGCGCGGT |
| | | CGGAAGAAGCTGCTGTACATCTTTAAGCAACCCTTCA |
| | | TGAGGCCTGTGCAGACTACTCAAGAGGAGGACGGCT |
| | | GTTCATGCCGGTTCCCAGAGGAGGAGGAAGGCGGCT |
| | | GCGAACTGCGCGTGAAATTCAGCCGCAGCGCAGATG |
| | | CTCCAGCCTACCAGCAGGGGCAGAACCAGCTCTACA |
| | | ACGAACTCAATCTTGGTCGGAGAGAGGAGTACGACG |
| | | TGCTGGACAAGCGGAGAGGACGGGACCCAGAAATGG |
| | | GCGGGAAGCCGCGCAGAAAGAATCCCCAAGAGGGCC |
| | | TGTACAACGAGCTCCAAAAGGATAAGATGGCAGAAG |
| | | CCTATAGCGAGATTGGTATGAAAGGGGAACGCAGAA |
| | | GAGGCAAAGGCCACGACGGACTGTACCAGGGACTCA |
| | | GCACCGCCACCAAGGACACCTATGACGCTCTTCACAT |
| | | GCAGGCCCTGCCGCCTCGG |
| CD20-C8H4 | | |
| SEQ ID NO: | HCDR1 | RYNMH |
| 2145 (Kabat) | | |
| SEQ ID NO: | HCDR2 | AIYPGNGDTSYSQKFKG |
| 2146 (Kabat) | | |
| SEQ ID NO: | HCDR3 | SFFYGSSDWYFDV |
| 2147 (Kabat) | | |
| SEQ ID NO: | HCDR1 | GYTFTRY |
| 2148 (Chothia) | | |
| SEQ ID NO: | HCDR2 | YPGNGD |
| 2047 (Chothia) | | |
| SEQ ID NO: | HCDR3 | SFFYGSSDWYFDV |
| 2147 (Chothia) | | |
| SEQ ID NO: | HCDR1 | GYTFTRYN |
| 2149 (IMGT) | | |
| SEQ ID NO: | HCDR2 | IYPGNGDT |
| 2049 (IMGT) | | |
| SEQ ID NO: | HCDR3 | ARSFFYGSSDWYFDV |
| 2150 (IMGT) | | |
| SEQ ID NO: | HCDR1 | GYTFTRYNMH |
| 2151 (Combined | | |
| Chothia and | | |
| Kabat) | | |
| SEQ ID NO: | HCDR2 | AIYPGNGDTSYSQKFKG |
| 2146 (Combined | | |
| Chothia and | | |
| Kabat) | | |
| SEQ ID NO: | HCDR3 | SFFYGSSDWYFDV |
| 2147 (Combined | | |
| Chothia and | | |
| Kabat) | | |
| SEQ ID NO: | VH | QVQLVQSGAEVKKPGSSVKVSCKASGYTFTRYNMHW |
| 2170 | | VRQAPGQGLEWMGAIYPGNGDTSYSQKFKGRVTITAD |
| | | KSTSTAYMELSSLRSEDTAVYYCARSFFYGSSDWYFDV |
| | | WGQGTTVTVSS |
| SEQ ID NO: | DNA VH | CAAGTCCAACTCGTCCAGTCTGGCGCAGAAGTCAAG |
| 2177 | | AAGCCCGGAAGCTCCGTGAAAGTGTCCTGCAAAGCG |
| | | TCGGGTTACACTTTCACCCGGTACAACATGCACTGGG |
| | | TCAGACAGGCCCCTGGACAAGGACTGGAGTGGATGG |
| | | GTGCCATCTACCCTGGAAACGGAGATACCTCCTACTC |
| | | CCAAAAGTTCAAGGGGAGAGTGACCATTACCGCCGA |
| | | CAAGTCAACTTCCACCGCTTACATGGAGCTCAGCTCC |
| | | CTGCGGTCCGAAGATACTGCGGTGTACTATTGCGCTC |
| | | GCTCATTTTTCTACGGCTCATCGGATTGGTACTTCGAC |
| | | GTCTGGGGACAGGGAACTACCGTGACCGTGTCCTCG |
| SEQ ID NO: | LCDR1 | RASSSVNNMH |
| 2154 (Kabat) | | |
| SEQ ID NO: | LCDR2 | ATSNLAS |
| 2031 (Kabat) | | |
| SEQ ID NO: | LCDR3 | QQWIFNPPT |
| 2055 (Kabat) | | |
| SEQ ID NO: | LCDR1 | SSSVNN |
| 2155 (Chothia) | | |
| SEQ ID NO: | LCDR2 | ATS |
| 2034 (Chothia) | | |
| SEQ ID NO: | LCDR3 | WIFNPP |
| 2057 (Chothia) | | |
| SEQ ID NO: | LCDR1 | SSVNN |
| 2156 (IMGT) | | |
| SEQ ID NO: | LCDR2 | ATS |
| 2034 (IMGT) | | |
| SEQ ID NO: | LCDR3 | QQWIFNPPT |
| 2055 (IMGT) | | |
| SEQ ID NO: | LCDR1 | RASSSVNNMH |
| 2154 (Combined | | |
| Chothia and | | |
| Kabat) | | |
| SEQ ID NO: | LCDR2 | ATSNLAS |
| 2031 (Combined | | |
| Chothia and | | |
| Kabat) | | |
| SEQ ID NO: | LCDR3 | QQWIFNPPT |
| 2055 (Combined | | |
| Chothia and | | |
| Kabat) | | |
| SEQ ID NO: | VL | DIQLTQSPSFLSASVGDRVTITCRASSSVNNMHWYQQK |
| 2164 | | PGKAPKPLIYATSNLASGVPSRFSGSGSGTEYTLTISSLQ |
| | | PEDFATYYCQQWIFNPPTFGQGTKLEIK |
| SEQ ID NO: | DNA VL | GACATCCAGCTGACTCAGTCCCCGTCCTTCCTGTCCG |
| 2178 | | CCTCCGTGGGGGACCGCGTGACGATTACTTGTCGGGC |
| | | CTCCTCATCCGTGAACAACATGCATTGGTACCAGCAG |
| | | AAGCCAGGAAAGGCACCGAAGCCGCTTATCTATGCC |
| | | ACCTCGAATCTGGCCAGCGGAGTGCCTTCGAGGTTTA |
| | | GCGGCTCCGGCTCCGGCACCGAGTACACTTTGACCAT |
| | | TAGCAGCCTCCAGCCGGAGGACTTCGCCACATACTAC |
| | | TGCCAGCAGTGGATCTTCAACCCCCCCACCTTCGGCC |
| | | AAGGAACCAAGCTGGAAATCAAG |
| SEQ ID NO: | Linker | GGGGSGGGGSGGGGSGGGGS |
| 1010 | | |
| SEQ ID NO: | scFv (VH- | QVQLVQSGAEVKKPGSSVKVSCKASGYTFTRYNMHW |
| 2179 | linker-VL) | VRQAPGQGLEWMGAIYPGNGDTSYSQKFKGRVTITAD |
| | | KSTSTAYMELSSLRSEDTAVYYCARSFFYGSSDWYFDV |
| | | WGQGTTVTVSSGGGGSGGGGSGGGGSGGGGSDIQLTQ |
| | | SPSFLSASVGDRVTITCRASSSVNNMHWYQQKPGKAPK |
| | | PLIYATSNLASGVPSRFSGSGSGTEYTLTISSLQPEDFAT |
| | | YYCQQWIFNPPTFGQGTKLEIK |
| SEQ ID NO: | DNA scFv | CAAGTCCAACTCGTCCAGTCTGGCGCAGAAGTCAAG |
| 2180 | (VH-linker- | AAGCCCGGAAGCTCCGTGAAAGTGTCCTGCAAAGCG |
| | VL) | TCGGGTTACACTTTCACCCGGTACAACATGCACTGGG |
| | | TCAGACAGGCCCCTGGACAAGGACTGGAGTGGATGG |
| | | GTGCCATCTACCCTGGAAACGGAGATACCTCCTACTC |
| | | CCAAAAGTTCAAGGGGAGAGTGACCATTACCGCCGA |
| | | CAAGTCAACTTCCACCGCTTACATGGAGCTCAGCTCC |
| | | CTGCGGTCCGAAGATACTGCGGTGTACTATTGCGCTC |
| | | GCTCATTTTTCTACGGCTCATCGGATTGGTACTTCGAC |
| | | GTCTGGGGACAGGGAACTACCGTGACCGTGTCCTCG |
| | | GGGGGAGGAGGATCGGGCGGAGGCGGTTCGGGAGG |
| | | CGGCGGAAGCGGAGGCGGAGGTTCAGACATCCAGCT |
| | | GACTCAGTCCCCGTCCTTCCTGTCCGCCTCCGTGGGG |
| | | GACCGCGTGACGATTACTTGTCGGGCCTCCTCATCCG |
| | | TGAACAACATGCATTGGTACCAGCAGAAGCCAGGAA |
| | | AGGCACCGAAGCCGCTTATCTATGCCACCTCGAATCT |
| | | GGCCAGCGGAGTGCCTTCGAGGTTTAGCGGCTCCGGC |
| | | TCCGGCACCGAGTACACTTTGACCATTAGCAGCCTCC |
| | | AGCCGGAGGACTTCGCCACATACTACTGCCAGCAGT |
| | | GGATCTTCAACCCCCCCACCTTCGGCCAAGGAACCAA |
| | | GCTGGAAATCAAG |
| SEQ ID NO: | Full CAR | MALPVTALLLPLALLLHAARPQVQLVQSGAEVKKPGSS |
| 2181 | amino acid | VKVSCKASGYTFTRYNMHWVRQAPGQGLEWMGAIYP |
| | sequence | GNGDTSYSQKFKGRVTITADKSTSTAYMELSSLRSEDT |
| | | AVYYCARSFFYGSSDWYFDVWGQGTTVTVSSGGGGSG |
| | | GGGSGGGGSGGGGSDIQLTQSPSFLSASVGDRVTITCRA |
| | | SSSVNNMHWYQQKPGKAPKPLIYATSNLASGVPSRFSG |
| | | SGSGTEYTLTISSLQPEDFATYYCQQWIFNPPTFGQGTK |
| | | LEIKTTTPAPRPPTPAPTIASQPLSLRPEACRPAAGGAVH |
| | | TRGLDFACDIYIWAPLAGTCGVLLLSLVITLYCKRGRKK |
| | | LLYIFKQPFMRPVQTTQEEDGCSCRFPEEEEGGCELRVK |
| | | FSRSADAPAYQQGQNQLYNELNLGRREEYDVLDKRRG |
| | | RDPEMGGKPRRKNPQEGLYNELQKDKMAEAYSEIGMK |
| | | GERRRGKGHDGLYQGLSTATKDTYDALHMQALPPR |
| SEQ ID NO: | Full CAR | ATGGCCCTCCCTGTCACCGCCCTGCTGCTTCCGCTGG |
| 2182 | nucleic acid | CTCTTCTGCTCCACGCCGCTCGGCCCCAAGTCCAACT |
| | sequence | CGTCCAGTCTGGCGCAGAAGTCAAGAAGCCCGGAAG |
| | | CTCCGTGAAAGTGTCCTGCAAAGCGTCGGGTTACACT |
| | | TTCACCCGGTACAACATGCACTGGGTCAGACAGGCCC |
| | | CTGGACAAGGACTGGAGTGGATGGGTGCCATCTACC |
| | | CTGGAAACGGAGATACCTCCTACTCCCAAAAGTTCAA |
| | | GGGGAGAGTGACCATTACCGCCGACAAGTCAACTTC |
| | | CACCGCTTACATGGAGCTCAGCTCCCTGCGGTCCGAA |
| | | GATACTGCGGTGTACTATTGCGCTCGCTCATTTTTCTA |
| | | CGGCTCATCGGATTGGTACTTCGACGTCTGGGGACAG |
| | | GGAACTACCGTGACCGTGTCCTCGGGGGGAGGGGGG |
| | | AGCGGCGGAGGGGGCTCGGGCGGTGGAGGAAGCGG |
| | | AGGCGGCGGTTCGGACATCCAGCTGACTCAGTCCCCG |
| | | TCCTTCCTGTCCGCCTCCGTGGGGGACCGCGTGACGA |
| | | TTACTTGTCGGGCCTCCTCATCCGTGAACAACATGCA |
| | | TTGGTACCAGCAGAAGCCAGGAAAGGCACCGAAGCC |
| | | GCTTATCTATGCCACCTCGAATCTGGCCAGCGGAGTG |
| | | CCTTCGAGGTTTAGCGGCTCCGGCTCCGGCACCGAGT |
| | | ACACTTTGACCATTAGCAGCCTCCAGCCGGAGGACTT |
| | | CGCCACATACTACTGCCAGCAGTGGATCTTCAACCCC |
| | | CCCACCTTCGGCCAAGGAACCAAGCTGGAAATCAAG |
| | | ACCACTACCCCAGCACCGAGGCCACCCACCCCGGCTC |
| | | CTACCATCGCCTCCCAGCCTCTGTCCCTGCGTCCGGA |
| | | GGCATGTAGACCCGCAGCTGGTGGGGCCGTGCATAC |
| | | CCGGGGTCTTGACTTCGCCTGCGATATCTACATTTGG |
| | | GCCCCTCTGGCTGGTACTTGCGGGGTCCTGCTGCTTT |
| | | CACTCGTGATCACTCTTTACTGTAAGCGCGGTCGGAA |
| | | GAAGCTGCTGTACATCTTTAAGCAACCCTTCATGAGG |
| | | CCTGTGCAGACTACTCAAGAGGAGGACGGCTGTTCAT |
| | | GCCGGTTCCCAGAGGAGGAGGAAGGCGGCTGCGAAC |
| | | TGCGCGTGAAATTCAGCCGCAGCGCAGATGCTCCAG |
| | | CCTACCAGCAGGGGCAGAACCAGCTCTACAACGAAC |
| | | TCAATCTTGGTCGGAGAGAGGAGTACGACGTGCTGG |
| | | ACAAGCGGAGAGGACGGGACCCAGAAATGGGCGGG |
| | | AAGCCGCGCAGAAAGAATCCCCAAGAGGGCCTGTAC |
| | | AACGAGCTCCAAAAGGATAAGATGGCAGAAGCCTAT |
| | | AGCGAGATTGGTATGAAAGGGGAACGCAGAAGAGGC |
| | | AAAGGCCACGACGGACTGTACCAGGGACTCAGCACC |
| | | GCCACCAAGGACACCTATGACGCTCTTCACATGCAGG |
| | | CCCTGCCGCCTCGG |
| CD20-C2 | | |
| SEQ ID NO: | VH | QVHLQQSGAELAKPGASVKMSCKASGYTFTNYWMHW |
| 2183 | | VKQRPGQGLEWIGFITPTTGYPEYNQKFKDKATLTADK |
| | | SSSTAYMQLSSLTSEDSAVYYCARRKVGKGVYYALDY |
| | | WGQGTSVTVSS |
| SEQ ID NO: | DNA VH | CAAGTGCATCTGCAGCAGTCGGGGGCCGAACTGGCA |
| 2184 | | AAGCCAGGCGCCAGCGTGAAGATGAGCTGCAAGGCC |
| | | TCCGGGTACACCTTCACCAACTACTGGATGCACTGGG |
| | | TCAAGCAGCGCCCGGGCCAGGGACTCGAGTGGATCG |
| | | GGTTCATCACGCCGACTACCGGCTACCCGGAGTATAA |
| | | CCAGAAGTTCAAGGACAAGGCCACTCTGACTGCCGA |
| | | CAAGTCCTCGTCTACCGCGTACATGCAACTGTCCTCA |
| | | CTGACTTCGGAGGATTCCGCTGTGTACTACTGCGCGC |
| | | GGAGGAAAGTCGGAAAGGGAGTGTACTATGCCCTGG |
| | | ACTACTGGGGCCAGGGTACCAGCGTCACTGTGTCCTC |
| | | C |
| SEQ ID NO: | VL | DILMTQSPASLSASVGETVTITCRASGNIHNYLAWYQQ |
| 2185 | | KQGNSPQLLVYNTKTLADGVPSRFSGSGSGTQYSLKINS |
| | | LQTEDFGTYYCQHFWSSPWTFGGGTKLEIK |
| SEQ ID NO: | DNA VL | GACATTCTGATGACCCAGTCCCCTGCATCACTCTCCG |
| 2186 | | CGTCCGTGGGAGAAACCGTGACCATCACGTGTAGAG |
| | | CCTCCGGCAACATCCACAACTACCTGGCCTGGTACCA |
| | | GCAGAAGCAGGGAAACTCGCCCCAACTGCTTGTGTA |
| | | CAACACCAAGACCTTGGCTGACGGAGTGCCTTCCCGG |
| | | TTCTCGGGTTCGGGATCAGGCACACAGTACTCCCTGA |
| | | AAATCAATAGCCTCCAGACCGAAGATTTTGGAACCTA |
| | | CTACTGCCAACACTTCTGGAGCTCCCCCTGGACTTTC |
| | | GGAGGCGGTACCAAGCTCGAGATTAAG |
| CD20-C3 | | |
| SEQ ID NO: | VH | QVQLQQPGAELVKPGASVKMSCKASGYTFTNYNLHW |
| 2187 | | VKQTPGQGLEWIGAIYPGNYDTSYNQKFKGKATLTAD |
| | | KSSSTAYMLLSSLTSEDSAVYFCARVDFGHSRYWYFDV |
| | | WGAGTTVTVSS |
| SEQ ID NO: | DNA VH | CAAGTGCAGCTGCAGCAGCCTGGTGCCGAGCTCGTG |
| 2188 | | AAGCCGGGAGCGTCCGTGAAGATGAGCTGCAAAGCC |
| | | TCGGGCTACACCTTCACCAATTACAACTTGCATTGGG |
| | | TCAAGCAGACCCCGGGCCAGGGCCTCGAATGGATCG |
| | | GAGCGATCTACCCCGGGAACTACGATACTAGCTACA |
| | | ACCAGAAGTTCAAGGGAAAGGCCACCCTGACCGCCG |
| | | ATAAGTCCTCATCCACCGCCTACATGCTGCTGTCCTC |
| | | GCTGACTTCCGAGGACTCCGCTGTGTACTTCTGCGCC |
| | | CGCGTGGACTTCGGACACAGCAGATATTGGTATTTTG |
| | | ACGTCTGGGGCGCCGGGACTACCGTGACTGTGTCGTC |
| | | C |
| SEQ ID NO: | VL | QIVLSQSPAILSASPGEKVTMTCRATSSVSSMNWYQQKP |
| 2189 | | GSFPRPWIHATSNLASGVPARFSGSGSGTSYSLTISRVEA |
| | | EDAATYYCQQWTFNPPTFGAGAKLELK |
| SEQ ID NO: | DNA VL | CAAATTGTCCTGAGCCAGAGCCCGGCTATCCTGTCCG |
| 2190 | | CCTCACCGGGCGAAAAGGTCACCATGACTTGTCGGG |
| | | CCACTTCCTCCGTGTCATCCATGAACTGGTACCAGCA |
| | | GAAGCCTGGCAGCTTCCCTCGGCCATGGATTCACGCC |
| | | ACGTCAAACCTGGCATCGGGAGTGCCCGCAAGGTTCT |
| | | CCGGGTCCGGCAGCGGAACATCCTACTCCCTCACCAT |
| | | CTCGCGCGTGGAAGCGGAGGACGCTGCCACCTACTA |
| | | CTGCCAACAGTGGACCTTCAACCCCCCCACCTTTGGA |
| | | GCGGGAGCCAAGCTGGAACTTAAG |
| CD20-C5 | | |
| SEQ ID NO: | VH | QVQLQQPGAELVKPGASVKMSCKASGYTFTSYNMHW |
| 2191 | | VKQTPGQGLEWIGAIYPGNGDTSYNPKFKGKATLTADK |
| | | SSRTAYIHLSSLTSEDSVVYYCARSYFYGSSSWYFDVW |
| | | GAGTTVTVSS |
| SEQ ID NO: | DNA VH | CAAGTGCAGCTGCAGCAGCCGGGAGCAGAGCTCGTG |
| 2192 | | AAGCCTGGAGCCTCAGTGAAGATGAGCTGCAAGGCC |
| | | TCCGGTTACACCTTCACCTCCTACAACATGCACTGGG |
| | | TCAAGCAGACCCCCGGACAAGGCCTGGAATGGATCG |
| | | GCGCCATCTACCCGGGAAACGGGGACACCTCCTATA |
| | | ACCCCAAGTTCAAGGGAAAAGCAACCCTGACCGCGG |
| | | ACAAGTCCAGCAGAACTGCCTACATCCATCTTTCCTC |
| | | GCTGACGTCCGAGGATTCCGTGGTGTACTACTGTGCC |
| | | CGCTCCTACTTCTACGGGTCATCCTCGTGGTACTTCG |
| | | ATGTCTGGGGCGCTGGAACCACCGTGACTGTGTCCTC |
| | | C |
| SEQ ID NO: | VL | QIILSQSPAILSASPGEKVTLTCRASSSVSSMHWYQQKPG |
| 2193 | | SSPKPWIFATSNLASGVPARFTGSGSGTSYSLTISRVEAE |
| | | DAATYYCQQWIFNPPTFGGGTSLEIK |
| SEQ ID NO: | DNA VL | CAGATCATTCTGAGCCAGAGCCCGGCCATTCTGTCTG |
| 2194 | | CCTCGCCTGGAGAAAAAGTCACCCTCACTTGCCGGGC |
| | | CAGCTCCTCCGTGTCCTCAATGCACTGGTACCAGCAG |
| | | AAGCCTGGCTCAAGCCCGAAGCCCTGGATCTTCGCCA |
| | | CCTCCAATCTGGCGTCAGGAGTGCCCGCGAGGTTCAC |
| | | TGGATCGGGGTCCGGCACATCGTATTCGCTCACCATT |
| | | TCCCGGGTGGAGGCCGAGGACGCCGCTACTTACTACT |
| | | GCCAACAGTGGATCTTCAACCCACCGACCTTTGGCGG |
| | | AGGGACTTCCTTGGAAATCAAG |
| CD20-C6 | | |
| SEQ ID NO: | VH | QIQLVQSGPELKKPGETVKISCKTSGYTFTSHGINWVKQ |
| 2195 | | APRKGLKWMGWINTYTGEPTYGDDFKGRFAFSLETSA |
| | | RTAYLQINNLKNEDTATYFCARYGNYEEPYAMDYWGQ |
| | | GTSVTVSS |
| SEQ ID NO: | DNA VH | CAAATTCAGCTGGTGCAGTCGGGACCTGAGCTCAAG |
| 2196 | | AAGCCCGGAGAAACCGTGAAGATCTCCTGCAAGACT |
| | | TCCGGGTACACTTTTACTTCCCACGGCATCAACTGGG |
| | | TCAAGCAGGCACCAAGGAAGGGGCTTAAGTGGATGG |
| | | GCTGGATTAACACCTACACCGGCGAACCCACCTATGG |
| | | CGATGACTTCAAAGGACGGTTCGCGTTCTCCCTCGAA |
| | | ACCTCAGCAAGAACCGCGTATTTGCAAATCAACAAC |
| | | CTGAAGAACGAGGACACCGCCACCTACTTCTGCGCCC |
| | | GCTACGGAAATTACGAGGAACCTTACGCTATGGACT |
| | | ACTGGGGCCAGGGCACTTCCGTGACTGTGTCGTCC |
| SEQ ID NO: | VL | QIVLSQSPAILSASPGEKVTMTCRATSSVSSMNWYQQKP |
| 2189 | | GSFPRPWIHATSNLASGVPARFSGSGSGTSYSLTISRVEA |
| | | EDAATYYCQQWTFNPPTFGAGAKLELK |
| SEQ ID NO: | DNA VL | CAGATCGTGCTGAGCCAGAGCCCCGCCATCCTGAGC |
| 2197 | | GCTTCCCCGGGAGAAAAGGTCACCATGACTTGCCGG |
| | | GCCACTAGCAGCGTGTCCTCCATGAACTGGTACCAGC |
| | | AGAAGCCGGGCTCCTTCCCTCGCCCCTGGATTCATGC |
| | | CACCTCAAACCTGGCCAGCGGAGTGCCAGCCAGATT |
| | | CTCGGGATCTGGATCGGGGACGTCCTACTCCCTCACC |
| | | ATCTCGCGGGTGGAGGCCGAAGATGCCGCCACATAC |
| | | TACTGTCAACAGTGGACCTTCAACCCGCCGACCTTTG |
| | | GAGCGGGGGCCAAGCTGGAGCTGAAA |
| CD20-C7 | | |
| SEQ ID NO: | VH | QVQLQQPGAELVKPGASVKMSCKASGYTFTSYNIHWV |
| 2198 | | KQTPGQGLEWIGAIYPGNGDTSYNQKFKGKATLTADKS |
| | | STTAFIHFSSLTSEDSVVYYCARSYFYGSDSWYFDVWG |
| | | AGTTVTVSS |
| SEQ ID NO: | DNA VH | CAAGTGCAGCTTCAGCAGCCTGGGGCCGAACTCGTG |
| 2199 | | AAGCCAGGAGCCTCCGTGAAGATGTCATGCAAAGCC |
| | | TCCGGCTACACTTTTACCTCCTACAACATTCATTGGGT |
| | | CAAGCAGACACCTGGCCAGGGCCTGGAATGGATTGG |
| | | TGCAATCTACCCGGGCAACGGAGACACCTCGTACAA |
| | | CCAGAAGTTTAAGGGGAAGGCCACCCTGACCGCGGA |
| | | CAAGTCAAGCACTACCGCGTTCATTCACTTCTCGTCC |
| | | TTGACCTCCGAGGATAGCGTGGTGTACTACTGCGCCC |
| | | GCTCCTATTTCTACGGCTCCGATTCGTGGTACTTCGAC |
| | | GTCTGGGGAGCCGGAACTACCGTGACCGTGTCCTCC |
| SEQ ID NO: | VL | QIILSQSPAILSASPGEKVTLTCRASSGVPSLHWYQQKPG |
| 2200 | | SSPKPWIFATSNLASGVPARFSGSGSGTSYSLTISRVEAE |
| | | DAATYYCQQWIFNPPTFGGGTSLEIK |
| SEQ ID NO: | DNA VL | CAAATCATCCTGAGCCAGAGCCCGGCCATCCTGTCGG |
| 2201 | | CTTCACCCGGGGAAAAGGTCACGCTGACTTGCCGGG |
| | | CCTCCTCCGGCGTGCCAAGCCTCCACTGGTACCAGCA |
| | | AAAGCCTGGCTCGTCCCCCAAACCCTGGATTTTCGCC |
| | | ACCTCCAACCTGGCTAGCGGAGTGCCGGCCAGATTCT |
| | | CGGGTTCCGGGTCCGGCACCAGCTATTCTCTCACCAT |
| | | CTCCCGGGTCGAGGCGGAGGACGCAGCGACTTACTA |
| | | CTGTCAACAGTGGATCTTCAATCCGCCCACCTTCGGC |
| | | GGAGGAACTTCCCTGGAAATCAAG |
| CD20-C8 | | |
| SEQ ID NO: | VH | QVQLLQPGAELVKPGASVKMSCKASGYTFTRYNMHW |
| 2202 | | VKQTPGQGLEWIGAIYPGNGDTSYSQKFKGKATLTADK |
| | | SSSTAYMQLSSLTSEDSAVYYCARSFFYGSSDWYFDVW |
| | | GAGTTVSVSS |
| SEQ ID NO: | DNA VH | CAAGTGCAGCTGCTGCAGCCCGGAGCCGAACTCGTG |
| 2203 | | AAGCCGGGCGCATCCGTGAAAATGAGCTGCAAGGCG |
| | | TCCGGTTACACCTTCACTCGCTACAACATGCACTGGG |
| | | TCAAGCAGACCCCTGGACAAGGCCTGGAGTGGATTG |
| | | GTGCTATCTACCCGGGAAACGGAGACACTAGCTACTC |
| | | GCAGAAATTCAAGGGAAAGGCCACGCTGACCGCCGA |
| | | TAAGTCCTCCTCCACTGCCTACATGCAACTCAGCTCA |
| | | CTGACCTCAGAGGACTCGGCCGTGTACTACTGCGCGA |
| | | GGTCCTTCTTCTACGGGTCCTCGGATTGGTACTTCGA |
| | | CGTCTGGGGCGCCGGTACCACCGTGTCCGTGTCATCC |
| SEQ ID NO: | VL | QIVLSQSPAILSTSPGEKVTLTCRASSSVNNMHWYQQKP |
| 2204 | | GSSPKPWIYATSNLASGVPSRFSGSGSGTSYSLTISRVEA |
| | | EDAATYYCQQWIFNPPTFGAGTKLELK |
| SEQ ID NO: | DNA VL | CAGATCGTGCTGAGCCAGTCCCCGGCGATTCTGTCCA |
| 2205 | | CCTCGCCTGGGGAAAAGGTCACCCTGACATGTAGAG |
| | | CCTCCTCCTCCGTGAACAATATGCATTGGTATCAGCA |
| | | GAAGCCAGGATCAAGCCCCAAGCCCTGGATCTATGC |
| | | CACTTCGAACCTTGCCTCTGGAGTGCCCTCACGGTTC |
| | | TCCGGCTCGGGATCGGGGACCAGCTACAGCTTGACTA |
| | | TCTCCCGGGTGGAGGCTGAGGACGCCGCAACCTACT |
| | | ACTGCCAGCAATGGATCTTCAACCCTCCGACTTTTGG |
| | | GGCCGGAACCAAGCTGGAACTCAAG |
| CD20-3m | | |
| SEQ ID NO: | VH | QVQLVESGGGVVQPGRSLRLSCAASGFTFRDYYMAWV |
| 2206 | | RQAPGKGLEWVASISYEGNPYYGDSVKGRFTISRDNAK |
| | | STLYLQMSSLRAEDTAVYYCARHDHNNVDWFAYWGQ |
| | | GTLVTV |
| SEQ ID NO: | DNA VH | CAAGTGCAGTTGGTGGAATCAGGAGGAGGTGTCGTG |
| 2207 | | CAACCAGGAAGATCATTGAGGCTCTCATGCGCCGCC |
| | | AGCGGATTCACCTTTCGGGATTACTACATGGCCTGGG |
| | | TCCGCCAGGCCCCGGGGAAGGGACTGGAATGGGTGG |
| | | CATCCATCTCGTACGAAGGGAACCCCTACTATGGGGA |
| | | CTCCGTGAAGGGACGGTTCACCATCTCCCGGGACAAC |
| | | GCCAAGTCCACCCTGTACCTTCAAATGTCCTCGCTGA |
| | | GGGCGGAGGATACTGCTGTCTACTACTGTGCCCGCCA |
| | | CGACCATAACAACGTGGACTGGTTCGCCTACTGGGGC |
| | | CAGGGAACCCTCGTCACCGTGTCCTCG |
| SEQ ID NO: | VL | DIVMTQTPLSLSVTPGQPVSMSCKSSQSLLYSENKKNYL |
| 2208 | | AWYLQKPGQSPQLLIFWASTRESGVPDRFSGSGSGTDFT |
| | | LKISRVEAEDVGVYYCQQYYNFPTFGQGTKLEIK |
| SEQ ID NO: | DNA VL | GACATTGTGATGACGCAGACTCCCCTGTCGCTCTCCG |
| 2209 | | TGACCCCTGGCCAGCCCGTGTCGATGTCGTGCAAGAG |
| | | CTCCCAGTCCCTGCTGTATTCCGAGAACAAGAAGAAT |
| | | TACCTTGCGTGGTACCTCCAGAAGCCGGGGCAGAGC |
| | | CCGCAGCTGCTGATTTTCTGGGCGTCCACTAGAGAGT |
| | | CTGGAGTGCCTGACCGGTTTAGCGGAAGCGGCTCCG |
| | | GTACTGATTTCACCCTGAAAATCTCGCGCGTGGAAGC |
| | | TGAGGACGTGGGCGTGTACTACTGCCAGCAGTACTAC |
| | | AACTTCCCTACTTTCGGACAAGGAACCAAGCTGGAA |
| | | ATCAAG |
| SEQ ID NO: | Linker | GGGGSGGGGSGGGGSGGGGS |
| 1010 | | |
| SEQ ID NO: | scFv (VH- | QVQLVESGGGVVQPGRSLRLSCAASGFTFRDYYMAWV |
| 2210 | linker-VL) | RQAPGKGLEWVASISYEGNPYYGDSVKGRFTISRDNAK |
| | | STLYLQMSSLRAEDTAVYYCARHDHNNVDWFAYWGQ |
| | | GTLVTVSSGGGGSGGGGSGGGGSGGGGSDIVMTQTPLS |
| | | LSVTPGQPVSMSCKSSQSLLYSENKKNYLAWYLQKPGQ |
| | | SPQLLIFWASTRESGVPDRFSGSGSGTDFTLKISRVEAED |
| | | VGVYYCQQYYNFPTFGQGTKLEIK |
| CD20-3J | | |
| SEQ ID NO: | VH | QVQLVQSGAEVKKPGASVKVSCKASGFTFRDYYMAW |
| 2211 | | VRQAPGQRLEWMGSISYEGNPYYGDSVKGRVTITRDNS |
| | | ASTLYMELSSLRSEDTAVYYCARHDHNNVDWFAYWG |
| | | QGTLVTVSS |
| SEQ ID NO: | DNA VH | CAAGTCCAACTCGTCCAGTCCGGTGCAGAAGTCAAG |
| 2212 | | AAACCAGGAGCTTCCGTGAAAGTGTCGTGCAAAGCT |
| | | TCAGGCTTCACCTTCCGCGACTATTACATGGCCTGGG |
| | | TCCGCCAAGCGCCCGGACAGCGGCTGGAGTGGATGG |
| | | GGTCCATTTCCTACGAGGGGAACCCCTACTATGGAGA |
| | | TTCCGTGAAGGGCAGAGTGACGATCACTCGGGATAA |
| | | CTCCGCCTCCACTCTCTACATGGAACTGTCCTCGCTTC |
| | | GGAGCGAAGATACCGCGGTGTACTACTGCGCCCGCC |
| | | ACGACCATAACAACGTGGACTGGTTCGCCTACTGGG |
| | | GACAGGGGACCCTCGTGACCGTGTCCTCT |
| SEQ ID NO: | VL | DIQMTQSPSSLSASVGDRVTITCKSSQSLLYSENKKNYL |
| 2213 | | AWYQQKPGKVPKLLIFWASTRESGVPSRFSGSGSGTDF |
| | | TLTISSLQPEDVATYYCQQYYNFPTFGQGTKLEIK |
| SEQ ID NO: | DNA VL | GACATTCAGATGACCCAGTCCCCGAGCTCGCTGTCCG |
| 2214 | | CCTCCGTGGGAGACAGAGTGACAATCACTTGCAAGA |
| | | GCAGCCAGTCACTGTTGTACTCCGAGAACAAGAAGA |
| | | ACTACCTCGCCTGGTACCAGCAGAAGCCGGGAAAGG |
| | | TCCCTAAGCTGCTGATCTTCTGGGCCAGCACTAGGGA |
| | | GTCGGGAGTGCCGTCACGGTTCAGCGGATCGGGATC |
| | | GGGTACCGACTTCACCCTGACTATCTCCTCCCTGCAA |
| | | CCTGAGGACGTGGCCACCTACTACTGTCAGCAGTACT |
| | | ACAATTTTCCCACCTTCGGCCAGGGTACCAAGCTGGA |
| | | AATCAAG |
| SEQ ID NO: | Linker | GGGGSGGGGSGGGGSGGGGS |
| 1010 | | |
| SEQ ID NO: | scFv (VH- | QVQLVQSGAEVKKPGASVKVSCKASGFTFRDYYMAW |
| 2215 | linker-VL) | VRQAPGQRLEWMGSISYEGNPYYGDSVKGRVTITRDNS |
| | | ASTLYMELSSLRSEDTAVYYCARHDHNNVDWFAYWG |
| | | QGTLVTVSSGGGGSGGGGSGGGGSGGGGSDIQMTQSPS |
| | | SLSASVGDRVTITCKSSQSLLYSENKKNYLAWYQQKPG |
| | | KVPKLLIFWASTRESGVPSRFSGSGSGTDFTLTISSLQPE |
| | | DVATYYCQQYYNFPTFGQGTKLEIK |
| CD20-3H5k1 | | |
| SEQ ID NO: | VH | EVQLVQSGAEVKKPGESLKISCKGSGFTFRDYYMAWV |
| 2216 | | RQMPGKGLEWMGSISYEGNPYYGDSVKGQVTISRDNSI |
| | | STLYLQWSSLKASDTAMYYCARHDHNNVDWFAYWGQ |
| | | GTLVTVSS |
| SEQ ID NO: | DNA VH | GAAGTCCAACTGGTGCAGTCAGGAGCAGAAGTCAAA |
| 2217 | | AAACCAGGAGAAAGCCTCAAGATCAGCTGCAAGGGC |
| | | TCGGGTTTCACCTTCCGGGACTACTATATGGCCTGGG |
| | | TCAGACAGATGCCGGGAAAGGGACTGGAATGGATGG |
| | | GGTCAATCAGCTACGAGGGCAACCCCTACTACGGAG |
| | | ACTCCGTGAAGGGACAGGTCACAATCTCCCGGGACA |
| | | ACTCGATTTCCACTCTGTATCTGCAATGGAGCTCCCT |
| | | CAAGGCCTCCGACACTGCGATGTACTACTGTGCGCGG |
| | | CATGACCACAACAATGTGGATTGGTTCGCCTACTGGG |
| | | GACAGGGAACCCTCGTGACCGTGTCCAGC |
| SEQ ID NO: | VL | DIQMTQSPSSLSASVGDRVTITCKSSQSLLYSENKKNYL |
| 2213 | | AWYQQKPGKVPKLLIFWASTRESGVPSRFSGSGSGTDF |
| | | TLTISSLQPEDVATYYCQQYYNFPTFGQGTKLEIK |
| SEQ ID NO: | DNA VL | GATATCCAAATGACCCAGTCGCCCTCCTCACTCTCCG |
| 2218 | | CCTCCGTGGGAGATCGCGTGACCATTACTTGCAAGAG |
| | | CTCGCAGTCCCTGCTGTACTCCGAGAACAAGAAGAA |
| | | CTACTTGGCCTGGTACCAGCAGAAGCCCGGCAAAGT |
| | | GCCGAAGCTGCTTATCTTTTGGGCCTCGACCAGGGAA |
| | | AGCGGAGTGCCGTCACGCTTCTCCGGCTCCGGGTCTG |
| | | GCACCGACTTCACTCTGACTATTTCCTCCCTGCAACCT |
| | | GAGGACGTGGCTACCTACTACTGCCAGCAGTACTACA |
| | | ACTTCCCTACCTTCGGCCAAGGGACGAAGCTGGAGAT |
| | | CAAG |
| SEQ ID NO: | Linker | GGGGSGGGGSGGGGSGGGGS |
| 1010 | | |
| SEQ ID NO: | scFv (VH- | EVQLVQSGAEVKKPGESLKISCKGSGFTFRDYYMAWV |
| 2219 | linker-VL) | RQMPGKGLEWMGSISYEGNPYYGDSVKGQVTISRDNSI |
| | | STLYLQWSSLKASDTAMYYCARHDHNNVDWFAYWGQ |
| | | GTLVTVSSGGGGSGGGGSGGGGSGGGGSDIQMTQSPSS |
| | | LSASVGDRVTITCKSSQSLLYSENKKNYLAWYQQKPGK |
| | | VPKLLIFWASTRESGVPSRFSGSGSGTDFTLTISSLQPED |
| | | VATYYCQQYYNFPTFGQGTKLEIK |
| CD20-3H5k3 | | |
| SEQ ID NO: | VH | EVQLVQSGAEVKKPGESLKISCKGSGFTFRDYYMAWV |
| 2216 | | RQMPGKGLEWMGSISYEGNPYYGDSVKGQVTISRDNSI |
| | | STLYLQWSSLKASDTAMYYCARHDHNNVDWFAYWGQ |
| | | GTLVTVSS |
| SEQ ID NO: | DNA VH | GAAGTGCAGTTGGTCCAATCAGGCGCAGAAGTGAAG |
| 2220 | | AAACCCGGAGAATCATTGAAGATTTCGTGCAAAGGA |
| | | AGCGGGTTCACATTCCGCGATTACTACATGGCGTGGG |
| | | TCAGACAGATGCCGGGAAAGGGACTCGAGTGGATGG |
| | | GGTCCATCAGCTACGAAGGAAACCCTTACTACGGGG |
| | | ACTCCGTGAAGGGCCAGGTCACCATCTCCCGCGACA |
| | | ACTCAATCTCCACTCTGTATCTGCAATGGTCGAGCCT |
| | | CAAGGCCTCTGATACTGCGATGTACTACTGCGCTCGG |
| | | CATGACCACAACAACGTGGACTGGTTCGCTTACTGGG |
| | | GACAGGGTACCCTTGTGACCGTGTCCTCC |
| SEQ ID NO: | VL | EIVMTQSPATLSLSPGERATLSCKSSQSLLYSENKKNYL |
| 2221 | | AWYQQKPGQAPRLLIFWASTRESGIPARFSGSGSGTDFT |
| | | LTISSLOPEDLAVYYCQQYYNFPTFGQGTKLEIK |
| SEQ ID NO: | DNA VL | GAGATCGTGATGACTCAGTCCCCTGCCACCCTCTCGC |
| 2222 | | TGTCCCCCGGGGAGAGGGCCACGCTGTCCTGCAAGA |
| | | GCTCCCAGTCACTGCTGTATTCCGAAAACAAGAAGA |
| | | ACTACCTCGCCTGGTACCAACAGAAGCCGGGACAGG |
| | | CCCCGCGGCTTCTGATCTTCTGGGCCTCCACTCGGGA |
| | | GTCCGGCATTCCGGCCCGCTTCTCCGGCTCGGGGAGC |
| | | GGAACTGACTTCACCCTGACCATCAGCAGCCTGCAGC |
| | | CAGAGGACCTCGCAGTGTACTACTGTCAACAGTACTA |
| | | CAATTTCCCCACCTTTGGCCAGGGTACCAAGCTGGAG |
| | | ATTAAG |
| SEQ ID NO: | Linker | GGGGSGGGGSGGGGSGGGGS |
| 1010 | | |
| SEQ ID NO: | scFv (VH- | EVQLVQSGAEVKKPGESLKISCKGSGFTFRDYYMAWV |
| 2223 | linker-VL) | RQMPGKGLEWMGSISYEGNPYYGDSVKGQVTISRDNSI |
| | | STLYLQWSSLKASDTAMYYCARHDHNNVDWFAYWGQ |
| | | GTLVTVSSGGGGSGGGGSGGGGSGGGGSEIVMTQSPAT |
| | | LSLSPGERATLSCKSSQSLLYSENKKNYLAWYQQKPGQ |
| | | APRLLIFWASTRESGIPARFSGSGSGTDFTLTISSLQP |
| | | EDLAVYYCQQYYNFPTFGQGTKLEIK |
| CD20-Ofa | | |
| SEQ ID NO: | HCDR1 | DYAMH |
| 1120 (Kabat) | | |
| SEQ ID NO: | HCDR2 | TISWNSGSIGYADSVKG |
| 2224 (Kabat) | | |
| SEQ ID NO: | HCDR3 | DIQYGNYYYGMDV |
| 2225 (Kabat) | | |
| SEQ ID NO: | HCDR1 | GFTFNDY |
| 2226 (Chothia) | | |
| SEQ ID NO: | HCDR2 | SWNSGS |
| 2227 (Chothia) | | |
| SEQ ID NO: | HCDR3 | DIQYGNYYYGMDV |
| 2225 (Chothia) | | |
| SEQ ID NO: | HCDR1 | GFTFNDYA |
| 2228 (IMGT) | | |
| SEQ ID NO: | HCDR2 | ISWNSGSI |
| 2229 (IMGT) | | |
| SEQ ID NO: | HCDR3 | AKDIQYGNYYYGMDV |
| 2230 (IMGT) | | |
| SEQ ID NO: | VH | EVOLVESGGGLVQPGRSLRLSCAASGFTFNDYAMHWV |
| 2231 | | RQAPGKGLEWVSTISWNSGSIGYADSVKGRFTISRDNA |
| | | KKSLYLQMNSLRAEDTALYYCAKDIQYGNYYYGMDV |
| | | WGQGTTVTVSS |
| SEQ ID NO: | DNA VH | GAGGTGCAGCTGGTCGAGTCGGGGGGAGGATTGGTG |
| 2232 | | CAGCCGGGCAGAAGCCTGCGGCTCTCATGTGCCGCCT |
| | | CCGGCTTCACCTTTAACGACTACGCAATGCACTGGGT |
| | | CAGACAGGCTCCTGGGAAGGGCCTGGAATGGGTGTC |
| | | CACCATTTCCTGGAACTCCGGGAGCATCGGCTACGCT |
| | | GACTCCGTGAAGGGCCGCTTCACGATTAGCCGCGATA |
| | | ACGCGAAAAAGAGCCTGTACCTCCAAATGAACTCCC |
| | | TGCGGGCCGAAGATACCGCCCTTTACTACTGCGCGAA |
| | | GGACATTCAGTATGGAAACTACTACTACGGAATGGA |
| | | CGTCTGGGGACAGGGGACCACAGTGACCGTGTCAAG |
| | | C |
| SEQ ID NO: | LCDRI | RASQSVSSYLA |
| 2233 (Kabat) | | |
| SEQ ID NO: | LCDR2 | DASNRAT |
| 1287 (Kabat) | | |
| SEQ ID NO: | LCDR3 | QQRSNWPIT |
| 2234 (Kabat) | | |
| SEQ ID NO: | LCDR1 | SQSVSSY |
| 2235 (Chothia) | | |
| SEQ ID NO: | LCDR2 | DAS |
| 2236 (Chothia) | | |
| SEQ ID NO: | LCDR3 | RSNWPI |
| 2237 (Chothia) | | |
| SEQ ID NO: | LCDR1 | QSVSSY |
| 2238 (IMGT) | | |
| SEQ ID NO: | LCDR2 | DAS |
| 2236 (IMGT) | | |
| SEQ ID NO: | LCDR3 | QQRSNWPIT |
| 2234 (IMGT) | | |
| SEQ ID NO: | VL | EIVLTQSPATLSLSPGERATLSCRASQSVSSYLAWYQQK |
| 2239 | | PGQAPRLLIYDASNRATGIPARFSGSGSGTDFTLTISSLEP |
| | | EDFAVYYCQQRSNWPITFGQGTRLEIK |
| SEQ ID NO: | DNA VL | GAAATCGTGCTGACCCAGAGCCCAGCCACTTTGTCAC |
| 2240 | | TGTCCCCCGGCGAAAGAGCCACTCTGTCCTGCCGGGC |
| | | ATCGCAGTCCGTGTCGTCCTACCTGGCCTGGTACCAG |
| | | CAAAAGCCCGGACAAGCCCCTCGCCTTCTCATCTACG |
| | | ACGCCTCCAATCGCGCGACCGGAATCCCGGCCAGGTT |
| | | CTCCGGGAGCGGTTCAGGCACTGACTTCACCCTGACC |
| | | ATCTCGTCCCTGGAGCCGGAGGATTTCGCCGTGTATT |
| | | ACTGCCAGCAGCGGTCCAACTGGCCCATCACCTTCGG |
| | | CCAAGGGACTCGGCTCGAAATCAAG |
| SEQ ID NO: | Linker | GGGGSGGGGSGGGGSGGGGS |
| 1010 | | |
| SEQ ID NO: | scFv (VH- | EVQLVESGGGLVQPGRSLRLSCAASGFTFNDYAMHWV |
| 2241 | linker-VL) | RQAPGKGLEWVSTISWNSGSIGYADSVKGRFTISRDNA |
| | | KKSLYLQMNSLRAEDTALYYCAKDIQYGNYYYGMDV |
| | | WGQGTTVTVSSGGGGSGGGGSGGGGSGGGGSEIVLTQ |
| | | SPATLSLSPGERATLSCRASQSVSSYLAWYQQKPGQAP |
| | | RLLIYDASNRATGIPARFSGSGSGTDFTLTISSLEPEDFAV |
| | | YYCQQRSNWPITFGQGTRLEIK |
| SEQ ID NO: | DNA scFv | GAGGTGCAGCTGGTCGAGTCGGGGGGAGGATTGGTG |
| 2242 | (VH-linker- | CAGCCGGGCAGAAGCCTGCGGCTCTCATGTGCCGCCT |
| | VL) | CCGGCTTCACCTTTAACGACTACGCAATGCACTGGGT |
| | | CAGACAGGCTCCTGGGAAGGGCCTGGAATGGGTGTC |
| | | CACCATTTCCTGGAACTCCGGGAGCATCGGCTACGCT |
| | | GACTCCGTGAAGGGCCGCTTCACGATTAGCCGCGATA |
| | | ACGCGAAAAAGAGCCTGTACCTCCAAATGAACTCCC |
| | | TGCGGGCCGAAGATACCGCCCTTTACTACTGCGCGAA |
| | | GGACATTCAGTATGGAAACTACTACTACGGAATGGA |
| | | CGTCTGGGGACAGGGGACCACAGTGACCGTGTCAAG |
| | | CGGCGGTGGAGGATCTGGCGGAGGAGGTTCCGGTGG |
| | | CGGTGGATCGGGAGGGGGAGGATCGGAAATCGTGCT |
| | | GACCCAGAGCCCAGCCACTTTGTCACTGTCCCCCGGC |
| | | GAAAGAGCCACTCTGTCCTGCCGGGCATCGCAGTCCG |
| | | TGTCGTCCTACCTGGCCTGGTACCAGCAAAAGCCCGG |
| | | ACAAGCCCCTCGCCTTCTCATCTACGACGCCTCCAAT |
| | | CGCGCGACCGGAATCCCGGCCAGGTTCTCCGGGAGC |
| | | GGTTCAGGCACTGACTTCACCCTGACCATCTCGTCCC |
| | | TGGAGCCGGAGGATTTCGCCGTGTATTACTGCCAGCA |
| | | GCGGTCCAACTGGCCCATCACCTTCGGCCAAGGGACT |
| | | CGGCTCGAAATCAAG |
| CD20-3 | | |
| SEQ ID NO: | VH | EVQLVESGGGLVQPGRSLKLSCAASGFTFRDYYMAWV |
| 2243 | | RQAPKKGLEWVASISYEGNPYYGDSVKGRFTISRNNAK |
| | | STLYLQMNSLRSEDTATYYCARHDHNNVDWFAYWGQ |
| | | GTLVTVSS |
| SEQ ID NO: | VL | DIVMTQTPSSQAVSAGEKVTMSCKSSQSLLYSENKKNY |
| 2244 | | LAWYQQKPGQSPKLLIFWASTRESGVPDRFIGSGSGTDF |
| | | TLTISSVQAEDLAVYYCQQYYNFPTFGSGTKLEIK |
| SEQ ID NO: | Linker | GGGGSGGGGSGGGGSGGGGS |
| 1010 | | |
| SEQ ID NO: | scFv (VH- | EVQLVESGGGLVQPGRSLKLSCAASGFTFRDYYMAWV |
| 2245 | linker-VL) | RQAPKKGLEWVASISYEGNPYYGDSVKGRFTISRNNAK |
| | | STLYLQMNSLRSEDTATYYCARHDHNNVDWFAYWGQ |
| | | GTLVTVSSGGGGSGGGGSGGGGSGGGGSDIVMTQTPSS |
| | | QAVSAGEKVTMSCKSSQSLLYSENKKNYLAWYQQKPG |
| | | QSPKLLIFWASTRESGVPDRFIGSGSGTDFTLTISSVQAE |
| | | DLAVYYCQQYYNFPTFGSGTKLEIK |
| CD20-8aBBz | | |
| SEQ ID NO: | VH | EVQLQQSGAELVKPGASVKMSCKASGYTFTSYNMHW |
| 2246 | | VKQTPGQGLEWIGAIYPGNGDTSYNQKFKGKATLTAD |
| | | KSSSTAYMQLSSLTSEDSADYYCARSNYYGSSYWFFDV |
| | | WGAGTTVTVSS |
| SEQ ID NO: | DNA VH | GAGGTGCAACTGCAGCAGTCAGGAGCAGAACTGGTC |
| 2247 | | AAGCCGGGCGCATCCGTCAAGATGAGCTGCAAGGCC |
| | | TCAGGATACACCTTCACTTCATACAACATGCACTGGG |
| | | TCAAGCAGACGCCTGGGCAGGGGCTGGAGTGGATCG |
| | | GTGCCATCTACCCCGGAAACGGCGACACCTCCTACAA |
| | | CCAGAAGTTCAAGGGAAAGGCCACCCTCACCGCTGA |
| | | TAAGTCCAGCAGCACCGCCTACATGCAACTGTCGTCC |
| | | CTGACTTCGGAGGACAGCGCTGACTACTATTGCGCCC |
| | | GCTCTAATTACTACGGTTCCTCCTACTGGTTCTTCGAC |
| | | GTGTGGGGCGCGGGTACCACTGTGACTGTCTCCAGC |
| SEQ ID NO: | VL | DIVLTQSPAILSASPGEKVTMTCRASSSVNYMDWYQKK |
| 2248 | | PGSSPKPWIYATSNLASGVPARFSGSGSGTSYSLTISRVE |
| | | AEDAATYYCQQWSFNPPTFGGGTKLEIK |
| SEQ ID NO: | DNA VL | GACATCGTGCTCACTCAGTCGCCCGCCATTCTGAGCG |
| 2249 | | CTAGCCCCGGCGAAAAGGTCACCATGACCTGTAGAG |
| | | CGTCATCCTCGGTGAACTACATGGACTGGTACCAGAA |
| | | GAAGCCGGGATCGAGCCCTAAGCCATGGATCTACGC |
| | | CACATCCAATCTGGCGTCCGGCGTGCCGGCCCGGTTC |
| | | AGCGGGAGCGGCTCAGGCACCTCCTATTCCCTCACCA |
| | | TCTCGAGAGTGGAGGCTGAGGATGCAGCCACGTACT |
| | | ACTGTCAGCAGTGGTCGTTCAACCCCCCAACCTTTGG |
| | | TGGTGGAACCAAGCTGGAAATCAAG |
| SEQ ID NO: | Linker | GSTSGGGSGGGSGGGGSS |
| 2250 | | |
| SEQ ID NO: | scFv (VH- | DIVLTQSPAILSASPGEKVTMTCRASSSVNYMDWYQKK |
| 2251 | linker-VL) | PGSSPKPWIYATSNLASGVPARFSGSGSGTSYSLTISRVE |
| | | AEDAATYYCQQWSFNPPTFGGGTKLEIKGSTSGGGSGG |
| | | GSGGGGSSEVQLQQSGAELVKPGASVKMSCKASGYTF |
| | | TSYNMHWVKQTPGQGLEWIGAIYPGNGDTSYNQKFKG |
| | | KATLTADKSSSTAYMQLSSLTSEDSADYYCARSNYYGS |
| | | SYWFFDVWGAGTTVTVSS |
| SEQ ID NO: | DNA scFv | GACATCGTGCTCACTCAGTCGCCCGCCATTCTGAGCG |
| 2252 | (VH-linker- | CTAGCCCCGGCGAAAAGGTCACCATGACCTGTAGAG |
| | VL) | CGTCATCCTCGGTGAACTACATGGACTGGTACCAGAA |
| | | GAAGCCGGGATCGAGCCCTAAGCCATGGATCTACGC |
| | | CACATCCAATCTGGCGTCCGGCGTGCCGGCCCGGTTC |
| | | AGCGGGAGCGGCTCAGGCACCTCCTATTCCCTCACCA |
| | | TCTCGAGAGTGGAGGCTGAGGATGCAGCCACGTACT |
| | | ACTGTCAGCAGTGGTCGTTCAACCCCCCAACCTTTGG |
| | | TGGTGGAACCAAGCTGGAAATCAAGGGAAGCACCTC |
| | | CGGCGGAGGTTCCGGAGGAGGGTCCGGAGGCGGAGG |
| | | CAGCTCCGAGGTGCAACTGCAGCAGTCAGGAGCAGA |
| | | ACTGGTCAAGCCGGGCGCATCCGTCAAGATGAGCTG |
| | | CAAGGCCTCAGGATACACCTTCACTTCATACAACATG |
| | | CACTGGGTCAAGCAGACGCCTGGGCAGGGGCTGGAG |
| | | TGGATCGGTGCCATCTACCCCGGAAACGGCGACACCT |
| | | CCTACAACCAGAAGTTCAAGGGAAAGGCCACCCTCA |
| | | CCGCTGATAAGTCCAGCAGCACCGCCTACATGCAACT |
| | | GTCGTCCCTGACTTCGGAGGACAGCGCTGACTACTAT |
| | | TGCGCCCGCTCTAATTACTACGGTTCCTCCTACTGGTT |
| | | CTTCGACGTGTGGGGCGCGGGTACCACTGTGACTGTC |
| | | TCCAGC |

In some embodiments, the antigen-binding domain comprises a HC CDR1, a HC CDR2, and a HC CDR3 of any heavy chain binding domain amino acid sequences listed in Table 9. In embodiments, the antigen-binding domain further comprises a LC CDR1, a LC CDR2, and a LC CDR3. In embodiments, the antigen-binding domain comprises a LC CDR1, a LC CDR2, and a LC CDR3 amino acid sequences listed in Table 9.

In some embodiments, the antigen-binding domain comprises one, two or all of LC CDR1, LC CDR2, and LC CDR3 of any light chain binding domain amino acid sequences listed in Table 9, and one, two or all of HC CDR1, HC CDR2, and HC CDR3 of any heavy chain binding domain amino acid sequences listed in Table 9.

In some embodiments, the CDRs are defined according to the Kabat numbering scheme, the Chothia numbering scheme, or a combination thereof.

### CD22 CAR and CD22-Binding Sequences

In some embodiments, the CAR or CAR-expressing cell described herein is a CD22 CAR-expressing cell (e.g., a cell expressing a CAR molecule that binds to human CD22). In some embodiments, the CD22 CAR-expressing cell includes an antigen-binding domain according to WO2016/164731 and PCT/US2017/055627, incorporated herein by reference. Exemplary CD22-binding sequences or CD22 CAR sequences are disclosed in, e.g., Tables 6A, 6B, 7A, 7B, 7C, 8A, 8B, 9A, 9B, 10A, and 10B of WO2016/164731 and Tables 6-10 of PCT/US2017/055627. In some embodiments, the CD22-binding sequences or CD22 CAR sequences comprise a CDR, variable region, scFv or full-length sequence of a CD22 CAR disclosed in PCT/US2017/055627 or WO2016/164731.

In embodiments, the CAR molecule comprises an antigen-binding domain that binds specifically to CD22 (CD22 CAR). In some embodiments, the antigen-binding domain targets human CD22. In some embodiments, the antigen-binding domain includes a single chain Fv sequence as described herein.

The sequences of exemplary human CD22 CAR are provided below. In some embodiments, a human CD22 CAR is CAR22-65.

Exemplary human CD22 CAR scFv sequence
(SEQ ID NO: 2253)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSSGGGGSGGGGSGGGGSQSALT
QPASASGSPGQSVTISCTGTSSDVGGYNYVSWYQQHPGKAPKLMIYDVS
NRPSGVSNRFSGSKSGNTASLTISGLQAEDEADYYCSSYTSSSTLYVFG
TGTQLTVL
Exemplary human CD22 CAR heavy chain variable
region
(SEQ ID NO: 2254)
EVQLQQSGPGLVKPSQTLSLTCAISGDSMLSNSDTWNWIRQSPSRGLEW
LGRTYHRSTWYDDYASSVRGRVSINVDTSKNQYSLQLNAVTPEDTGVYY
CARVRLQDGNSWSDAFDVWGQGTMVTVSS
Exemplary human CD22 CAR light chain variable
region
(SEQ ID NO: 2255)
QSALTQPASASGSPGQSVTISCTGTSSDVGGYNYVSWYQQHPGKAPKLM
IYDVSNRPSGVSNRFSGSKSGNTASLTISGLQAEDEADYYCSSYTSSST
LYVFGTGTQLTVL

TABLE 10
Exemplary Heavy Chain Variable Domain CDRs of CD22 CAR (CAR22-65)

| | | SEQ ID | | SEQ ID | | SEQ ID |
|---|---|---|---|---|---|---|
| Candidate | HCDR1 | NO: | HCDR2 | NO: | HCDR3 | NO: |
| CAR22-65 | GDSML | 2256 | RTYHRSTWYDDYA | 2258 | VRLQDGNSWS | 2259 |
| Combined | SNSDTWN | | SSVRG | | DAFDV | |
| CAR22-65 | SNSDT | 2257 | RTYHRSTWYDDYA | 2258 | VRLQDGNSWS | 2259 |
| Kabat | WN | | SSVRG | | DAFDV | |

TABLE 11
Exemplary Light Chain Variable Domain CDRs of
CD22 CAR (CAR22-65). The LC CDR sequences in
this table have the same sequence under the
Kabat or combined definitions.

| | | SEQ | | SEQ | | SEQ |
|---|---|---|---|---|---|---|
| | | ID | | ID | | ID |
| Candidate | LCDR1 | NO: | LCDR2 | NO: | LCDR3 | NO: |
| CAR22-65 | TGTSSDVGGY | 2260 | DVSNRPS | 2261 | SSYTSSS | 2262 |
| Combined | NYVS | | | | TLYV | |

In some embodiments, the antigen-binding domain comprises a HC CDR1, a HC CDR2, and a HC CDR3 of any heavy chain binding domain amino acid sequences listed in Table 10. In embodiments, the antigen-binding domain further comprises a LC CDR1, a LC CDR2, and a LC CDR3. In embodiments, the antigen-binding domain comprises a LC CDR1, a LC CDR2, and a LC CDR3 amino acid sequences listed in Table 11.

In some embodiments, the antigen-binding domain comprises one, two or all of LC CDR1, LC CDR2, and LC CDR3 of any light chain binding domain amino acid sequences listed in Table 11, and one, two or all of HC CDR1, HC CDR2, and HC CDR3 of any heavy chain binding domain amino acid sequences listed in Table 10.

In some embodiments, the CDRs are defined according to the Kabat numbering scheme, the Chothia numbering scheme, or a combination thereof.

The order in which the VL and VH domains appear in the scFv can be varied (i.e., VL-VH, or VH-VL orientation), and where any of one, two, three or four copies of the “G4S” (SEQ ID NO: 1039) subunit, in which each subunit comprises the sequence GGGGS (SEQ ID NO: 1039) (e.g., (G4S)₃ (SEQ ID NO: 1011) or (G4S)₄(SEQ ID NO: 1010)), can connect the variable domains to create the entirety of the scFv domain. Alternatively, the CAR construct can include, for example, a linker including the sequence GSTSGSGKPGSGEGSTKG (SEQ ID NO: 2263). Alternatively, the CAR construct can include, for example, a linker including the sequence LAEAAAK (SEQ ID NO: 2264). In some embodiments, the CAR construct does not include a linker between the VL and VH domains.

These clones all contained a Q/K residue change in the signal domain of the co-stimulatory domain derived from CD3zeta chain.

### RNA Transfection

Disclosed herein are methods for producing an in vitro transcribed RNA CAR molecule. The present disclosure also includes a CAR construct encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases (SEQ ID NO: 1468) in length. RNA so produced can efficiently transfect different kinds of cells. In some embodiments, the template includes sequences for the CAR construct.

In some embodiments, the CAR molecule is encoded by a messenger RNA (mRNA). In some embodiments, the mRNA encoding the CAR molecule is introduced into an immune effector cell, e.g., a T cell or a NK cell, for production of a CAR-expressing cell (e.g., CAR T cell or CAR-expressing NK cell).

In some embodiments, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a CAR of the present disclosure. For example, the template for the RNA CAR comprises an extracellular region comprising a single chain variable domain of an anti-tumor antibody; a hinge region, a transmembrane domain (e.g., a transmembrane domain of CD8a); and a cytoplasmic region that includes an intracellular signaling domain, e.g., comprising the signaling domain of CD3-zeta and the signaling domain of 4-1BB.

In some embodiments, the DNA to be used for PCR contains an open reading frame. The DNA can be from a naturally occurring DNA sequence from the genome of an organism. In some embodiments, the nucleic acid can include some or all of the 5′ and/or 3′ untranslated regions (UTRs). The nucleic acid can include exons and introns. In some embodiments, the DNA to be used for PCR is a human nucleic acid sequence. In some embodiments, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In some embodiments, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In some embodiments, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments, the 5′ UTR can be 5′UTR of an RNA virus whose RNA genome is stable in cells. In other embodiments, various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 1469) (size can be 50-5000 T (SEQ ID NO: 1470)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In some embodiments, the poly(A) tail is between 100 and 5000 adenosines (SEQ ID NO: 1471).

Poly(A) tails of RNAs can be further extended following in vitro transcription with the use of a poly(A) polymerase, such as E. coli polyA polymerase (E-PAP). In some embodiments, increasing the length of a poly(A) tail from 100 nucleotides to between 300 and 400 nucleotides (SEQ ID NO: 1472) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

Non-viral delivery methods

In some embodiments, non-viral methods can be used to deliver a nucleic acid encoding a CAR molecule described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams. Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3(2013):1829-47; and Singh et al. Cancer Res. 68.8(2008): 2961-2971, all of which are incorporated herein by reference. Exemplary transposases include a Tc 1/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR molecule described herein. Provided herein are methods of generating a cell, e.g., T cell or NK cell, that stably expresses a CAR molecule described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR molecule described herein. In some embodiments, the nucleic acid contains a transposon comprising a transgene (e.g., a nucleic acid encoding a CAR molecule described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR molecule described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Nucleic Acid Constructs Encoding CAR Molecules

The present disclosure also provides nucleic acid molecules encoding one or more CAR constructs described herein. In some embodiments, the nucleic acid molecule is provided as a messenger RNA transcript. In some embodiments, the nucleic acid molecule is provided as a DNA construct.

Accordingly, in some embodiments, the disclosure pertains to an isolated nucleic acid molecule encoding a CAR molecule, wherein the CAR molecule comprises an antigen-binding domain, a transmembrane domain, and an intracellular signaling domain comprising a stimulatory domain, e.g., a costimulatory signaling domain and/or a primary signaling domain, e.g., zeta chain.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR molecule. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In some embodiments, the vector comprising the nucleic acid encoding the desired CAR molecule of the disclosure is an adenoviral vector (A5/35). In some embodiments, the expression of nucleic acids encoding CAR IL-15R/IL-15 can be accomplished using of transposons such as sleeping beauty, CRISPR, CAS9, and zinc finger nucleases. See below June et al. 2009Nature Reviews Immunology 9.10: 704-716, is incorporated herein by reference.

In brief summary, the expression of natural or synthetic nucleic acids CAR is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009).

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1 promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided below.

### Exemplary WT PGK Promoter

(SEQ ID NO: 1473)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCGCCTCGTCCTTCGCAGCGGC
CCCCCGGGTGTTCCCATCGCCGCTTCTAGGCCCACTGCGACGCTTGCCT
GCACTTCTTACACGCTCTGGGTCCCAGCCGCGGCGACGCAAAGGGCCTT
GGTGCGGGTCTCGTCGGCGCAGGGACGCGTTTGGGTCCCGACGGAACCT
TTTCCGCGTTGGGGTTGGGGCACCATAAGCT

### Exemplary Truncated PGK Promoters:

PGK100:
(SEQ ID NO: 1474)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTG
PGK200:
(SEQ ID NO: 1475)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACG
PGK300:
(SEQ ID NO: 1476)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCG
PGK400:
(SEQ ID NO: 1477)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCGCCTCGTCCTTCGCAGCGGC
CCCCCGGGTGTTCCCATCGCCGCTTCTAGGCCCACTGCGACGCTTGCCT
GCACTTCTTACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vector can further comprise a nucleic acid encoding a second CAR molecule. In some embodiments, the second CAR molecule includes an antigen binding domain to a target expressed on acute myeloid leukemia cells, such as, e.g., CD123, CD34, CLL-1, folate receptor beta, or FLT3; or a target expressed on a B cell, e.g., CD10, CD19, CD20, CD22, CD34, CD123, FLT-3, ROR1, CD79b, CD179b, or CD79a. In some embodiments, the vector comprises a nucleic acid sequence encoding a first CAR molecule that specifically binds a first antigen and includes an intracellular signaling domain having a costimulatory signaling domain but not a primary signaling domain, and a nucleic acid encoding a second CAR molecule that specifically binds a second, different, antigen and includes an intracellular signaling domain having a primary signaling domain but not a costimulatory signaling domain.

In some embodiments, the vector comprises a nucleic acid encoding a CAR molecule described herein and a nucleic acid encoding an inhibitory CAR molecule. In some embodiments, the inhibitory CAR molecule comprises an antigen-binding domain that binds an antigen found on normal cells but not cancer cells. In some embodiments, the inhibitory CAR molecule comprises the antigen-binding domain, a transmembrane domain and an intracellular domain of an inhibitory molecule. For example, the intracellular domain of the inhibitory CAR molecule can be an intracellular domain of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta.

In embodiments, the vector may comprise two or more nucleic acid sequences encoding a CAR molecule, e.g., a CAR molecule described herein and a second CAR molecule, e.g., an inhibitory CAR molecule or a CAR molecule that specifically binds to a different antigen. In such embodiments, the two or more nucleic acid sequences encoding the CAR molecule are encoded by a single nucleic molecule in the same frame and as a single polypeptide chain. In some embodiments, the two or more CAR molecules, can, e.g., be separated by one or more peptide cleavage sites. (e.g., an auto-cleavage site or a substrate for an intracellular protease). Examples of peptide cleavage sites include the following, wherein the GSG residues are optional:

T2A:
(SEQ ID NO: 1478)
(GSG)EGRGSLLTCGDVEENPGP
P2A:
(SEQ ID NO: 1479)
(GSG)ATNFSLLKQAGDVEENPGP
E2A:
(SEQ ID NO: 1480)
(GSG) QCTNYALLKLAGDVESNPGP
F2A:
(SEQ ID NO: 1481)
(GSG)VKQTLNFDLLKLAGDVESNPGP

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

The present disclosure further provides a vector comprising a CAR molecule-encoding nucleic acid molecule. In some embodiments, a CAR vector can be directly transduced into a cell, e.g., a T cell or NK cell. In some embodiments, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In some embodiments, the vector is a multicistronic vector. In some embodiments, the vector is capable of expressing the CAR construct in mammalian T cells or NK cells. In some embodiments, the mammalian T cell is a human T cell. In some embodiments, the mammalian NK cell is a human NK cell. In some embodiments, the T cell is autologous. In some embodiments, the T cell is allogeneic.

### Sources of Cells

Prior to expansion and genetic modification, a source of cells, e.g., immune effector cells (e.g., T cells or NK cells), is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain embodiments of the present disclosure, any number of immune effector cell (e.g., T cell or NK cell) lines available in the art, may be used. In certain embodiments of the present disclosure, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments of the disclosure, the cells are washed with phosphate buffered saline (PBS). In some embodiments, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational-Immunology (2015) 4, e31; doi:10.1038/cti.2014.31.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD4+, CD8+, CD45RA+, and/or CD45RO+ T cells, can be further isolated by positive or negative selection techniques. For example, in some embodiments, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least 1, 2, 3, 4, 5, or 6 hours. In some embodiments, the time period is 10 to 24 hours. In some embodiments, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection could also be used in the context of this disclosure. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. In certain embodiments, it may be desirable to enrich for cells that are CD127low. Alternatively, in certain embodiments, T regulatory cells are depleted by anti-C25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In some embodiments, T regulatory cells, e.g., CD25+ T cells, are removed from the population using an anti-CD25 antibody, or fragment thereof, or a CD25-binding ligand, IL-2. In some embodiments, the anti-CD25 antibody, or fragment thereof, or CD25-binding ligand is conjugated to a substrate, e.g., a bead, or is otherwise coated on a substrate, e.g., a bead. In some embodiments, the anti-CD25 antibody, or fragment thereof, is conjugated to a substrate as described herein.

In some embodiments, the T regulatory cells, e.g., CD25+ T cells, are removed from the population using CD25 depletion reagent from Miltenyi™. In some embodiments, the ratio of cells to CD25 depletion reagent is le7 cells to 20 uL, or le7 cells tol5 uL, or le7 cells to 10 uL, or le7 cells to 5 uL, or le7 cells to 2.5 uL, or le7 cells to 1.25 uL. In some embodiments, e.g., for T regulatory cells, e.g., CD25+ depletion, greater than 500 million cells/ml is used. In some embodiments, a concentration of cells of 600, 700, 800, or 900 million cells/ml is used.

In some embodiments, the population of immune effector cells to be depleted includes about 6×10⁹ CD25+ T cells. In other embodiments, the population of immune effector cells to be depleted include about 1×10⁹ to 1×10¹⁰ CD25+ T cell, and any integer value in between. In some embodiments, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In some embodiments, the T regulatory cells, e.g., CD25+ cells, are removed from the population using the CliniMAC system with a depletion tubing set, such as, e.g., tubing 162-01. In some embodiments, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In some embodiments, a subject is pre-treated with one or more therapies that reduce T_{REG} cells prior to collection of cells for CAR-expressing cell product manufacturing, thereby reducing the risk of subject relapse to CAR-expressing cell treatment. In some embodiments, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In some embodiments, a subject is pre-treated with cyclophosphamide prior to collection of cells for CAR IL-15R/IL-15-expressing cell product manufacturing, thereby reducing the risk of subject relapse to CAR IL-15R/IL-15-expressing cell treatment. In some embodiments, a subject is pre-treated with an anti-GITR antibody prior to collection of cells for CAR IL-15R/IL-15-expressing cell product manufacturing, thereby reducing the risk of subject relapse to CAR IL-15R/IL-15-expressing cell treatment.

In some embodiments, the population of cells to be removed are neither the regulatory T cells or tumor cells, but cells that otherwise negatively affect the expansion and/or function of CAR IL-15R/IL-15 T cells, e.g. cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR molecule, e.g., a CAR molecule described herein. In some embodiments, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta. In embodiments, the checkpoint inhibitor is PD1 or PD-L1. In some embodiments, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

In some embodiments, a T cell population can be selected that expresses one or more of IFN-γ, TNFα, IL-17A, IL-2, IL-3, IL-4, GM-CSF, IL-1β, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain embodiments, it may be desirable to significantly decrease the volume in which beads and cells are mixed together (e.g., increase the concentration of cells), to ensure maximum contact of cells and beads. For example, in some embodiments, a concentration of 2 billion cells/ml is used. In some embodiments, a concentration of 1 billion cells/ml is used. In some embodiments, greater than 100 million cells/ml is used. In some embodiments, a concentration of cells of 10, 15, 20, 25, 30, 35, 40, 45, or 50 million cells/ml is used. In yet some embodiments, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10e6/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as immune effector cells, e.g., T cells or NK cells, isolated and frozen for later use in cell therapy, e.g., T cell therapy, for any number of diseases or conditions that would benefit from cell therapy, e.g., T cell therapy, such as those described herein. In some embodiments, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the immune effector cells (e.g., T cells or NK cells) may be expanded, frozen, and used at a later time. In certain embodiments, samples are collected from a patient shortly after diagnosis of a particular disease as described herein but prior to any treatments. In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In some embodiments of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, the immune effector cells expressing a CAR molecule, e.g., a CAR molecule described herein, are obtained from a subject that has received a low, immune enhancing dose of an mTOR inhibitor. In some embodiments, the population of immune effector cells, e.g., T cells, to be engineered to express a CAR molecule, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR molecule, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In some embodiments, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In some embodiments, the NK cells are obtained from the subject. In some embodiments, the NK cells are an NK cell line, e.g., NK-92 cell line (Conkwest).

### Modifications of CAR Cells, Including Allogeneic CAR Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II, and/or beta-2 microglobulin (β₂m). Compositions of allogeneic CAR and methods thereof have been described in, e.g., pages 227-237 of WO 2016/014565, incorporated herein by reference in its entirety.

In some embodiments, a cell, e.g., a T cell or a NK cell, is modified to reduce the expression of a TCR, and/or HLA, and/or β₂m, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), using, e.g., a method described herein, e.g., siRNA, shRNA, clustered regularly interspaced short palindromic repeats (CRISPR) transcription-activator like effector nuclease (TALEN), or zinc finger endonuclease (ZFN).

In some embodiments, a cell, e.g., a T cell or a NK cell is engineered to express a telomerase subunit, e.g., the catalytic subunit of telomerase, e.g., TERT, e.g., hTERT. In some embodiments, such modification improves persistence of the cell in a patient.

### Methods of CAR Manufacture/Production

The present disclosure also provides methods of making a cell disclosed herein, e.g., methods of engineering a T cell or NK cell to express a nucleic acid molecule encoding a CAR construct described herein, e.g., CD19 CAR construct. In some embodiments, provided herein is a population of cells (for example, immune effector cells, for example, T cells or NK cells) made by any of the manufacturing processes described herein.

### Activation Process

In some embodiments, the methods disclosed herein may manufacture immune effector cells engineered to express a CAR in less than 24 hours. Without wishing to be bound by theory, the methods provided herein preserve the undifferentiated phenotype of T cells, such as naïve T cells, during the manufacturing process. These CAR-expressing cells with an undifferentiated phenotype may persist longer and/or expand better in vivo after infusion. In some embodiments, CART cells produced by the manufacturing methods provided herein comprise a higher percentage of stem cell memory T cells, compared to CART cells produced by the traditional manufacturing process, e.g., as measured using scRNA-seq. In some embodiments, CART cells produced by the manufacturing methods provided herein comprise a higher percentage of effector T cells, compared to CART cells produced by the traditional manufacturing process, e.g., as measured using scRNA-seq. In some embodiments, CART cells produced by the manufacturing methods provided herein better preserve the stemness of T cells, compared to CART cells produced by the traditional manufacturing process, e.g., as measured using scRNA-seq. In some embodiments, CART cells produced by the manufacturing methods provided herein show a lower level of hypoxia, compared to CART cells produced by the traditional manufacturing process, e.g., as measured using scRNA-seq. In some embodiments, CART cells produced by the manufacturing methods provided herein show a lower level of autophagy, compared to CART cells produced by the traditional manufacturing process, e.g., as measured using scRNA-seq.

In some embodiments, the methods disclosed herein do not involve using a bead, such as Dynabeads® (for example, CD3/CD28 Dynabeads®), and do not involve a de-beading step. In some embodiments, the CART cells manufactured by the methods disclosed herein may be administered to a subject with minimal ex vivo expansion, for example, less than 1 day, less than 12 hours, less than 8 hours, less than 6 hours, less than 4 hours, less than 3 hours, less than 2 hours, less than 1 hour, or no ex vivo expansion. Accordingly, the methods described herein provide a fast manufacturing process of making improved CAR-expressing cell products for use in treating a disease in a subject.

In some embodiments, the present disclosure provides methods of making a population of cells (for example, T cells) that express a chimeric antigen receptor (CAR) comprising: (i) contacting a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a nucleic acid molecule(s) (for example, a DNA or RNA molecule) encoding the CAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, or 24 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 hours after the beginning of step (ii), for example, no later than 22, 23, 24, 25, 26, 27, 28, 29, or 30 hours after the beginning of step (ii); or (c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) a viral vector(s) comprising a nucleic acid molecule encoding the CAR.

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. Then the frozen apheresis sample is thawed, and T cells (for example, CD4+ T cells and/or CD8+ T cells) are selected from the apheresis sample, for example, using a cell-sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the activation process described herein.

In some embodiments, cells (for example, T cells) are contacted with anti-CD3 and anti-CD28 antibodies for, for example, 12 hours, followed by transduction with a vector (for example, a lentiviral vector) (e.g. one or more vectors) encoding a CAR. 24 hours after culture initiation, the cells are washed and formulated for storage or administration.

Without wishing to be bound by theory, brief CD3 and CD28 stimulation may promote efficient transduction of self-renewing T cells. Compared to traditional CART manufacturing approaches, the activation process provided herein does not involve prolonged ex vivo expansion. Similar to the cytokine process, the activation process provided herein also preserves undifferentiated T cells during CART manufacturing.

In some embodiments, the population of cells is contacted with an agent that stimulates a CD3/TCR complex and/or an agent that stimulates a costimulatory molecule on the surface of the cells.

In some embodiments, the agent that stimulates a CD3/TCR complex is an agent that stimulates CD3. In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally existing, recombinant, or chimeric ligand). In some embodiments, the agent that stimulates a CD3/TCR complex is an antibody. In some embodiments, the agent that stimulates a CD3/TCR complex is an anti-CD3 antibody. In some embodiments, the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally existing, recombinant, or chimeric ligand). In some embodiments, the agent that stimulates a costimulatory molecule is an antibody. In some embodiments, the agent that stimulates a costimulatory molecule is an anti-CD28 antibody. In some embodiments, the agent that stimulates a CD3/TCR complex or the agent that stimulates a costimulatory molecule does not comprise a bead. In some embodiments, the agent that stimulates a CD3/TCR complex comprises an anti-CD3 antibody covalently attached to a colloidal polymeric nanomatrix. In some embodiments, the agent that stimulates a costimulatory molecule comprises an anti-CD28 antibody covalently attached to a colloidal polymeric nanomatrix. In some embodiments, the agent that stimulates a CD3/TCR complex and the agent that stimulates a costimulatory molecule comprise T Cell TransAct™.

In some embodiments, the matrix comprises or consists of a polymeric, for example, biodegradable or biocompatible inert material, for example, which is non-toxic to cells. In some embodiments, the matrix is composed of hydrophilic polymer chains, which obtain maximal mobility in aqueous solution due to hydration of the chains. In some embodiments, the mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. Other polymers may include polyesters, polyethers, polyacrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. In some embodiments, the mobile matrix is a polymer of dextran.

In some embodiments, the population of cells is contacted with a nucleic acid molecule encoding a CAR. In some embodiments, the population of cells is transduced with a DNA molecule encoding a CAR.

In some embodiments, contacting the population of cells with the nucleic acid molecule(s) encoding the CAR occurs simultaneously with contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 20 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 19 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 18 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 17 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 16 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 15 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 13 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 12 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 11 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 10 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 9 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 8 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 7 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 6 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 4 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 3 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR occurs no later than 2 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 1 hour after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR occurs no later than 30 minutes after the beginning of contacting the population of cells with the agent that stimulates a CD3/TCR complex and/or the agent that stimulates a costimulatory molecule on the surface of the cells described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 26 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 25 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 24 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 23 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 22 hours after the beginning of contacting the population of cells with the agent that stimulates a CD3/TCR complex and/or the agent that stimulates a costimulatory molecule on the surface of the cells described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 5%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 10%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 15%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 20%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 25%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 30%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 35%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 40%, for example, as assessed by the number of living cells, compared to the population of cells before it is contacted with the agent that stimulates a CD3/TCR complex and/or the agent that stimulates a costimulatory molecule on the surface of the cells described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the activation process is conducted in serum free cell media. In some embodiments, the activation process is conducted in cell media comprising one or more cytokines chosen from: IL-2, IL-15 (for example, hetIL-15 (IL15/sIL-15Ra)), or IL-6 (for example, IL-6/sIL-6Ra). In some embodiments, hetIL-15 comprises the amino acid sequence of NWVNVISDLKKIEDLIQSMHIDATLYTESDVHPSCKVTAMKCFLLELQVISLESGDASIHDTV ENLIILANNSLSSNGNVTESGCKECEELEEKNIKEFLQSFVHIVQMFINTSITCPPPMSVEHADI WVKSYSLYSRERYICNSGFKRKAGTSSLTECVLNKATNVAHWTTPSLKCIRDPALVHQRPAP PSTVTTAGVTPQPESLSPSGKEPAASSPSSNNTAATTAAIVPGSQLMPSKSPSTGTTEISSHESS HGTPSQTTAKNWELTASASHQPPGVYPQG (SEQ ID NO: 3003). In some embodiments, hetIL-15 comprises an amino acid sequence having at least about 70, 75, 80, 85, 90, 95, or 99% identity to SEQ ID NO: 3003. In some embodiments, the activation process is conducted in cell media comprising a LSD1 inhibitor. In some embodiments, the activation process is conducted in cell media comprising a MALT1 inhibitor. In some embodiments, the serum free cell media comprises a serum replacement. In some embodiments, the serum replacement is CTS™ Immune Cell Serum Replacement (ICSR). In some embodiments, the level of ICSR can be, for example, up to 5%, for example, about 1%, 2%, 3%, 4%, or 5%.

In some embodiments, the present disclosure provides methods of making a population of cells (for example, T cells) that express a chimeric antigen receptor (CAR) comprising: (a) providing an apheresis sample (for example, a fresh or cryopreserved leukapheresis sample) collected from a subject; (b) selecting T cells from the apheresis sample (for example, using negative selection, positive selection, or selection without beads); (c) seeding isolated T cells at, for example, 1×10⁶ to 1×10⁷ cells/mL; (d) contacting T cells with an agent that stimulates T cells, for example, an agent that stimulates a CD3/TCR complex and/or an agent that stimulates a costimulatory molecule on the surface of the cells (for example, contacting T cells with anti-CD3 and/or anti-CD28 antibody, for example, contacting T cells with TransAct); (e) contacting T cells with a nucleic acid molecule(s) (for example, a DNA or RNA molecule) encoding the CAR (for example, contacting T cells with a virus comprising a nucleic acid molecule(s) encoding the CAR) for, for example, 6-48 hours, for example, 20-28 hours; and (f) washing and harvesting T cells for storage (for example, reformulating T cells in cryopreservation media) or administration. In some embodiments, step (f) is performed no later than 30 hours after the beginning of step (d) or (e), for example, no later than 22, 23, 24, 25, 26, 27, 28, 29, or 30 hours after the beginning of step (d) or (e).

In some embodiments, provided herein is a population of cells (for example, immune effector cells, for example, T cells or NK cells) made by any of the manufacturing processes described herein (e.g., the Activation Process described herein).

In some embodiments, the percentage of naïve cells, for example, naïve T cells, for example, CD45RA+CD45RO− CCR7+ T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15%, from, or (3) is increased, for example, by at least 5, 6, 7, 8, 9, 10, 11, 12, 13, 14, 15, 16, 17, 18, 19, 20, 21, 22, 23, 24, or 25%, as compared to, the percentage of naïve cells, for example, naïve T cells, for example, CD45RA+CD45RO− CCR7+ cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a higher percentage of naïve cells, for example, naïve T cells, for example, CD45RA+CD45RO− CCR7+ T cells (for example, at least 5, 6, 7, 8, 9, 10, 11, 12, 13, 14, 15, 16, 17, 18, 19, 20, 25, 30, 35, 40, 45, or 50% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of naïve cells, for example, naïve T cells, for example, CD45RA+CD45RO− CCR7+ T cells, in the population of cells at the end of the manufacturing process (for example, at the end of the cytokine process or the activation process described herein) is not less than 20, 25, 30, 35, 40, 45, 50, 55, or 60%.

In some embodiments, the percentage of central memory cells, for example, central memory T cells, for example, CD95+ central memory T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15% from, or (3) is decreased, for example, by at least 5, 6, 7, 8, 9, 10, 11, 12, 13, 14, 15, 16, 17, 18, 19, 20, 21, 22, 23, 24, or 25%, as compared to, the percentage of central memory cells, for example, central memory T cells, for example, CD95+ central memory T cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a lower percentage of central memory cells, for example, central memory T cells, for example, CD95+ central memory T cells (for example, at least 5, 6, 7, 8, 9, 10, 11, 12, 13, 14, 15, 16, 17, 18, 19, 20, 25, 30, 35, 40, 45, or 50% lower), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of central memory cells, for example, central memory T cells, for example, CD95+ central memory T cells, in the population of cells at the end of the manufacturing process (for example, at the end of the cytokine process or the activation process described herein) is no more than 40, 45, 50, 55, 60, 65, 70, 75, or 80%.

In some embodiments, the population of cells at the end of the manufacturing process (for example, at the end of the cytokine process or the activation process described herein) after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the population of cells has been enriched for IL6R-expressing cells (for example, cells that are positive for IL6Rx and/or IL6Rβ) prior to the beginning of the manufacturing process (for example, prior to the beginning of the cytokine process or the activation process described herein). In some embodiments, the population of cells comprises, for example, no less than 30, 35, 40, 45, 50, 55, 60, 65, 70, 75, or 80% of IL6R-expressing cells (for example, cells that are positive for IL6Rα and/or IL6Rβ) at the beginning of the manufacturing process (for example, at the beginning of the cytokine process or the activation process described herein).

### Cytokine Process

In some embodiments, the present disclosure provides methods of making a population of cells (for example, T cells) that express a chimeric antigen receptor (CAR) comprising: (1) contacting a population of cells with a cytokine chosen from IL-2, IL-7, IL-15, IL-21, IL-6, or a combination thereof, (2) contacting the population of cells (for example, T cells) with a nucleic acid molecule(s) (for example, a DNA or RNA molecule) encoding the CAR, thereby providing a population of cells (for example, T cells) comprising the nucleic acid molecule, and (3) harvesting the population of cells (for example, T cells) for storage (for example, reformulating the population of cells in cryopreservation media) or administration, wherein: (a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, or 24 hours after the beginning of step (1), for example, no later than 24 hours after the beginning of step (1), or (b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the nucleic acid molecule in step (2) is a DNA molecule. In some embodiments, the nucleic acid molecule in step (2) is an RNA molecule. In some embodiments, the nucleic acid molecule in step (2) is on a viral vector, for example, a viral vector chosen from a lentivirus vector, an adenoviral vector, or a retrovirus vector. In some embodiments, the nucleic acid molecule in step (2) is on a non-viral vector. In some embodiments, the nucleic acid molecule in step (2) is on a plasmid. In some embodiments, the nucleic acid molecule in step (2) is not on any vector. In some embodiments, step (2) comprises transducing the population of cells (for example, T cells) with a viral vector comprising a nucleic acid molecule(s) encoding the CAR.

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. The frozen apheresis sample is then thawed, and T cells (for example, CD4+ T cells and/or CD8+ T cells) are selected from the apheresis sample, for example, using a cell-sorting machine (for example, a CliniMACS® Prodigy® device). The selected T cells (for example, CD4+ T cells and/or CD8+ T cells) are then seeded for CART manufacturing using the cytokine process described herein. In some embodiments, at the end of the cytokine process, the CAR T cells are cryopreserved and later thawed and administered to the subject. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell-sorting machine (for example, a CliniMACS® Prodigy® device). The selected T cells (for example, CD4+ T cells and/or CD8+ T cells) are then seeded for CART manufacturing using the cytokine process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the cytokine process described herein.

In some embodiments, after cells (for example, T cells) are seeded, one or more cytokines (for example, one or more cytokines chosen from IL-2, IL-7, IL-15 (for example, hetIL-15 (IL15/sIL-15Ra)), IL-21, or IL-6 (for example, IL-6/sIL-6R)) as well as a vector (for example, a lentiviral vector) (e.g. one or more vectors) encoding a CAR are added to the cells. After incubation for 20-24 hours, the cells are washed and formulated for storage or administration.

Different from traditional CART manufacturing approaches, the cytokine process provided herein does not involve CD3 and/or CD28 stimulation, or ex vivo T cell expansion. T cells that are contacted with anti-CD3 and anti-CD28 antibodies and expanded extensively ex vivo tend to show differentiation towards a central memory phenotype. Without wishing to be bound by theory, the cytokine process provided herein preserves or increases the undifferentiated phenotype of T cells during CART manufacturing, generating a CART product that may persist longer after being infused into a subject.

In some embodiments, the population of cells is contacted with one or more cytokines (for example, one or more cytokines chosen from IL-2, IL-7, IL-15 (for example, hetIL-15 (IL15/sIL-15Ra)), IL-21, or IL-6 (for example, IL-6/sIL-6Ra).

In some embodiments, the population of cells is contacted with IL-2. In some embodiments, the population of cells is contacted with IL-7. In some embodiments, the population of cells is contacted with IL-15 (for example, hetIL-15 (IL15/sIL-15Ra)). In some embodiments, the population of cells is contacted with IL-21. In some embodiments, the population of cells is contacted with IL-6 (for example, IL-6/sIL-6Ra). In some embodiments, the population of cells is contacted with IL-2 and IL-7. In some embodiments, the population of cells is contacted with IL-2 and IL-15 (for example, hetIL-15 (IL15/sIL-15Ra)). In some embodiments, the population of cells is contacted with IL-2 and IL-21. In some embodiments, the population of cells is contacted with IL-2 and IL-6 (for example, IL-6/sIL-6Ra). In some embodiments, the population of cells is contacted with IL-7 and IL-15 (for example, hetIL-15 (IL15/sIL-15Ra)). In some embodiments, the population of cells is contacted with IL-7 and IL-21. In some embodiments, the population of cells is contacted with IL-7 and IL-6 (for example, IL-6/sIL-6Ra). In some embodiments, the population of cells is contacted with IL-15 (for example, hetIL-15 (IL15/sIL-15Ra)) and IL-21. In some embodiments, the population of cells is contacted with IL-15 (for example, hetIL-15 (IL15/sIL-15Ra)) and IL-6 (for example, IL-6/sIL-6Ra). In some embodiments, the population of cells is contacted with IL-21 and IL-6 (for example, IL-6/sIL-6Ra). In some embodiments, the population of cells is contacted with IL-7, IL-15 (for example, hetIL-15 (IL15/sIL-15Ra)), and IL-21. In some embodiments, the population of cells is further contacted with a LSD1 inhibitor. In some embodiments, the population of cells is further contacted with a MALT1 inhibitor.

In some embodiments, the population of cells is contacted with 20, 30, 40, 50, 60, 70, 80, 90, 100, 110, 120, 130, 140, 150, 160, 170, 180, 190, 200, 210, 220, 230, 240, 250, 260, 270, 280, 290, or 300 U/ml of IL-2. In some embodiments, the population of cells is contacted with 1, 2, 3, 4, 5, 6, 7, 8, 9, 10, 11, 12, 13, 14, 15, 16, 17, 18, 19, or 20 ng/ml of IL-7. In some embodiments, the population of cells is contacted with 1, 2, 3, 4, 5, 6, 7, 8, 9, 10, 11, 12, 13, 14, 15, 16, 17, 18, 19, or 20 ng/ml of IL-15.

In some embodiments, the population of cells is contacted with a nucleic acid molecule (e.g. one or more nucleic acid molecules) encoding a CAR. In some embodiments, the population of cells is transduced with a DNA molecule encoding a CAR.

In some embodiments, contacting the population of cells with the nucleic acid molecule encoding the CAR occurs simultaneously with contacting the population of cells with the one or more cytokines described above. In some embodiments, contacting the population of cells with the nucleic acid molecule encoding the CAR occurs no later than 1, 1.5, 2, 2.5, 3, 3.5, 4, 4.5, 5, 5.5, 6, 6.5, 7, 7.5, 8, 8.5, 9, 9.5 or 10 hours after the beginning of contacting the population of cells with the one or more cytokines described above. In some embodiments, contacting the population of cells with the nucleic acid molecule encoding the CAR occurs no later than 5 hours after the beginning of contacting the population of cells with the one or more cytokines described above. In some embodiments, contacting the population of cells with the nucleic acid molecule encoding the CAR occurs no later than 4 hours after the beginning of contacting the population of cells with the one or more cytokines described above.

In some embodiments, contacting the population of cells with the nucleic acid molecule encoding the CAR occurs no later than 3 hours after the beginning of contacting the population of cells with the one or more cytokines described above. In some embodiments, contacting the population of cells with the nucleic acid molecule encoding the CAR occurs no later than 2 hours after the beginning of contacting the population of cells with the one or more cytokines described above. In some embodiments, contacting the population of cells with the nucleic acid molecule encoding the CAR occurs no later than 1 hour after the beginning of contacting the population of cells with the one or more cytokines described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the one or more cytokines described above. In some embodiments, the population of cells is harvested for storage or administration no later than 26 hours after the beginning of contacting the population of cells with the one or more cytokines described above. In some embodiments, the population of cells is harvested for storage or administration no later than 25 hours after the beginning of contacting the population of cells with the one or more cytokines described above. In some embodiments, the population of cells is harvested for storage or administration no later than 24 hours after the beginning of contacting the population of cells with the one or more cytokines described above. In some embodiments, the population of cells is harvested for storage or administration no later than 23 hours after the beginning of contacting the population of cells with the one or more cytokines described above. In some embodiments, the population of cells is harvested for storage or administration no later than 22 hours after the beginning of contacting the population of cells with the one or more cytokines described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the one or more cytokines described above. In some embodiments, the population of cells is expanded by no more than 5%, for example, as assessed by the number of living cells, compared to the population of cells before it is contacted with the one or more cytokines described above. In some embodiments, the population of cells is expanded by no more than 10%, for example, as assessed by the number of living cells, compared to the population of cells before it is contacted with the one or more cytokines described above. In some embodiments, the population of cells is expanded by no more than 15%, for example, as assessed by the number of living cells, compared to the population of cells before it is contacted with the one or more cytokines described above. In some embodiments, the population of cells is expanded by no more than 20%, for example, as assessed by the number of living cells, compared to the population of cells before it is contacted with the one or more cytokines described above. In some embodiments, the population of cells is expanded by no more than 25%, for example, as assessed by the number of living cells, compared to the population of cells before it is contacted with the one or more cytokines described above. In some embodiments, the population of cells is expanded by no more than 30%, for example, as assessed by the number of living cells, compared to the population of cells before it is contacted with the one or more cytokines described above. In some embodiments, the population of cells is expanded by no more than 35%, for example, as assessed by the number of living cells, compared to the population of cells before it is contacted with the one or more cytokines described above. In some embodiments, the population of cells is expanded by no more than 40%, for example, as assessed by the number of living cells, compared to the population of cells before it is contacted with the one or more cytokines described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the population of cells is not contacted in vitro with an agent that stimulates a CD3/TCR complex (for example, an anti-CD3 antibody) and/or an agent that stimulates a costimulatory molecule on the surface of the cells (for example, an anti-CD28 antibody), or if contacted, the contacting step is less than 1, 1.5, 2, 2.5, 3, 3.5, 4, 4.5, or 5 hours.

In some embodiments, the population of cells is contacted in vitro with an agent that stimulates a CD3/TCR complex (for example, an anti-CD3 antibody) and/or an agent that stimulates a costimulatory molecule on the surface of the cells (for example, an anti-CD28 antibody) for 20, 21, 22, 23, 24, 25, 26, 27, or 28 hours.

In some embodiments, the population of cells manufactured using the cytokine process provided herein shows a higher percentage of naïve cells among CAR-expressing cells (for example, at least 5, 6, 7, 8, 9, 10, 11, 12, 13, 14, 15, 16, 17, 18, 19, 20, 25, 30, 35, 40, 45, 50, 55, or 60% higher), compared with cells made by an otherwise similar method which further comprises contacting the population of cells with, for example, an agent that binds a CD3/TCR complex (for example, an anti-CD3 antibody) and/or an agent that binds a costimulatory molecule on the surface of the cells (for example, an anti-CD28 antibody).

In some embodiments, the cytokine process provided herein is conducted in cell media comprising no more than 0, 0.5, 1, 1.5, 2, 2.5, 3, 3.5, 4, 4.5, 5, 5.5, 6, 6.5, 7, 7.5, or 8% serum. In some embodiments, the cytokine process provided herein is conducted in cell media comprising a LSD1 inhibitor, a MALT1 inhibitor, or a combination thereof.

### Additional Exemplary Manufacturing Methods

In some embodiments, cells, e.g., T cells or NK cells are activated, e.g., using anti-CD3/anti-CD28 antibody coated Dynabeads®, contacted with one or more nucleic acid molecules encoding a CAR and then expanded in vitro for, for example, 7, 8, 9, 10, or 11 days. In some embodiments, the cells, e.g., T cells or NK cells are selected from a fresh or cryopreserved leukapheresis sample, e.g., using positive or negative selection. In some embodiments, the cells are contacted with a nucleic acid molecule encoding a CAR, e.g., a CD19 CAR.

### Elutriation

In some embodiments, the methods described herein feature an elutriation method that removes unwanted cells, for example, monocytes and blasts, thereby resulting in an improved enrichment of desired immune effector cells suitable for CAR expression. In some embodiments, the elutriation method described herein is optimized for the enrichment of desired immune effector cells suitable for CAR expression from a previously frozen sample, for example, a thawed sample. In some embodiments, the elutriation method described herein provides a preparation of cells with improved purity as compared to a preparation of cells collected from the elutriation protocols known in the art. In some embodiments, the elutriation method described herein includes using an optimized viscosity of the starting sample, for example, cell sample, for example, thawed cell sample, by dilution with certain isotonic solutions (for example, PBS), and using an optimized combination of flow rates and collection volume for each fraction collected by an elutriation device. Exemplary elutriation methods that could be applied in the present disclosure are described on pages 48-51 of WO 2017/117112, herein incorporated by reference in its entirety.

### Density Gradient Centrifugation

Manufacturing of adoptive cell therapeutic product requires processing the desired cells, for example, immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product. For example, a loss of T cells in the final product, with a relative gain of non-T cells, especially undesirable B cells, blast cells and monocytes was observed in cell preparations isolated by density gradient centrifugation through Ficoll solution.

Without wishing to be bound by theory, it is believed that immune effector cells, for example, T cells, dehydrate during cryopreservation to become denser than fresh cells. Without wishing to be bound by theory, it is also believed that immune effector cells, for example, T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, for example, 1.077 g/mL.

In some embodiments, the density gradient centrifugation method described herein includes the use of a density gradient medium comprising iodixanol. In some embodiments, the density gradient medium comprises about 60% iodixanol in water.

In some embodiments, the density gradient centrifugation method described herein includes the use of a density gradient medium having a density greater than Ficoll. In some embodiments, the density gradient centrifugation method described herein includes the use of a density gradient medium having a density greater than 1.077 g/mL, for example, greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In some embodiments, the density gradient medium has a density of about 1.32 g/mL.

Additional embodiments of density gradient centrifugation are described on pages 51-53 of WO 2017/117112, herein incorporated by reference in its entirety.

### Enrichment by Selection

Provided herein are methods for selection of specific cells to improve the enrichment of the desired immune effector cells suitable for CAR expression. In some embodiments, the selection comprises a positive selection, for example, selection for the desired immune effector cells. In some embodiments, the selection comprises a negative selection, for example, selection for unwanted cells, for example, removal of unwanted cells. In embodiments, the positive or negative selection methods described herein are performed under flow conditions, for example, by using a flow-through device, for example, a flow-through device described herein. Exemplary positive and negative selections are described on pages 53-57 of WO 2017/117112, herein incorporated by reference in its entirety. Selection methods can be performed under flow conditions, for example, by using a flow-through device, also referred to as a cell processing system, to further enrich a preparation of cells for desired immune effector cells, for example, T cells, suitable for CAR expression. Exemplary flow-through devices are described on pages 57-70 of WO 2017/117112, herein incorporated by reference in its entirety. Exemplary cell separation and debeading methods are described on pages 70-78 of WO 2017/117112, herein incorporated by reference in its entirety.

Selection procedures are not limited to ones described on pages 57-70 of WO 2017/117112. Negative T cell selection via removal of unwanted cells with CD19, CD14 and CD26 Miltenyi beads in combination with column technology (CliniMACS® Plus or CliniMACS® Prodigy©) or positive T cell selection with a combination of CD4 and CD8 Miltenyi beads and column technology (CliniMACS® Plus or CliniMACS® Prodigy©) can be used. Alternatively, column-free technology with releasable CD3 beads (GE Healthcare) can be used.

In addition, bead-free technologies such as ThermoGenesis X-series devices can be utilized as well.

### Clinical Applications

All of the processes herein may be conducted according to clinical good manufacturing practice (cGMP) standards.

The processes may be used for cell purification, enrichment, harvesting, washing, concentration or for cell media exchange, particularly during the collection of raw, starting materials (particularly cells) at the start of the manufacturing process, as well as during the manufacturing process for the selection or expansion of cells for cell therapy.

The cells may include any plurality of cells. The cells may be of the same cell type, or mixed cell types. In addition, the cells may be from one donor, such as an autologous donor or a single allogenic donor for cell therapy. The cells may be obtained from patients by, for example, leukapheresis or apheresis. The cells may include T cells, for example may include a population that has greater than 50% T cells, greater than 60% T cells, greater than 70% T cells, greater than 80% T cells, or 90% T cells.

Selection processes may be particularly useful in selecting cells prior to culture and expansion. For instance, paramagnetic particles coated with anti-CD3 and/or anti CD28 may be used to select T cells for expansion or for introduction of a nucleic acid encoding a chimeric antigen receptor (CAR) or other protein. Such a process is used to produce CTL019 T cells for treatment of acute lymphoblastic leukemia (ALL).

The debeading processes and modules disclosed herein may be particularly useful in the manufacture of cells for cell therapy, for example in purifying cells prior to, or after, culture and expansion. For instance, paramagnetic particles coated with anti-CD3 and/or anti CD28 antibodies may be used to selectively expand T cells, for example T cells that are, or will be, modified by introduction of a nucleic acid encoding a chimeric antigen receptor (CAR) or other protein, such that the CAR is expressed by the T cells. During the manufacture of such T cells, the debeading processes or modules may be used to separate T cells from the paramagnetic particles. Such a debeading process or module is used to produce, for example, CTL019 T cells for treatment of acute lymphoblastic leukemia (ALL).

In one such process, illustrated here by way of example, cells, for example, T cells, are collected from a donor (for example, a patient to be treated with an autologous chimeric antigen receptor T cell product) via apheresis (for example, leukapheresis). Collected cells may then be optionally purified, for example, by an elutriation step, or via positive or negative selection of target cells (for example, T cells). Paramagnetic particles, for example, anti-CD3/anti-CD28-coated paramagnetic particles, may then be added to the cell population, to expand the T cells. The process may also include a transduction step, wherein nucleic acid encoding one or more desired proteins, for example, a CAR, for example a CAR targeting CD19, is introduced into the cell. The nucleic acid may be introduced in a lentiviral vector. The cells, for example, the lentivirally transduced cells, may then be expanded for a period of days, for example, 1, 2, 3, 4, 5, 6, 7, 8, 9, 10, or more days, for example in the presence of a suitable medium. After expansion, the debeading processes/modules disclosed herein may be used to separate the desired T cells from the paramagnetic particles. The process may include one or more debeading steps according to the processes of the present disclosure. The debeaded cells may then be formulated for administration to the patient. Examples of CAR T cells and their manufacture are further described, for example, in WO2012/079000, which is incorporated herein by reference in its entirety. The systems and methods of the present disclosure may be used for any cell separation/purification/debeading processes described in or associated with WO2012/079000. Additional CAR T manufacturing processes are described in, for example, WO2016109410 and WO2017117112, herein incorporated by reference in their entireties.

The systems and methods herein may similarly benefit other cell therapy products by wasting fewer desirable cells, causing less cell trauma, and more reliably removing magnetic and any non-paramagnetic particles from cells with less or no exposure to chemical agents, as compared to conventional systems and methods.

Although only exemplary embodiments of the disclosure are specifically described above, it will be appreciated that modifications and variations of these examples are possible without departing from the spirit and intended scope of the disclosure. For example, the magnetic modules and systems containing them may be arranged and used in a variety of configurations in addition to those described. Besides, non-magnetic modules can be utilized as well. In addition, the systems and methods may include additional components and steps not specifically described herein. For instance, methods may include priming, where a fluid is first introduced into a component to remove bubbles and reduce resistance to cell suspension or buffer movement. Furthermore, embodiments may include only a portion of the systems described herein for use with the methods described herein. For example, embodiments may relate to disposable modules, hoses, etc. usable within non-disposable equipment to form a complete system able to separate or debead cells to produce a cell product.

Additional manufacturing methods and processes that can be combined with the present disclosure have been described in the art. For example, pages 86-91 of WO 2017/117112 describe improved wash steps and improved manufacturing process.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the disclosure may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the costimulatory signal is bound to a cell surface and the agent providing the primary activation signal is in solution or coupled to a surface. In certain embodiments, both agents can be in solution. In some embodiments, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present disclosure.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof, and both agents are co-immobilized to the same bead in equivalent molecular amounts. In some embodiments, a 1:1 ratio of each antibody bound to the beads for CD4+ T cell expansion and T cell growth is used. In certain embodiments of the present disclosure, a ratio of anti CD3:CD28 antibodies bound to the beads is used such that an increase in T cell expansion is observed as compared to the expansion observed using a ratio of 1:1. In some embodiments, an increase of from about 1 to about 3 fold is observed as compared to the expansion observed using a ratio of 1:1. In some embodiments, the ratio of CD3:CD28 antibody bound to the beads ranges from 100:1 to 1:100 and all integer values there between. In some embodiments of the present disclosure, more anti-CD28 antibody is bound to the particles than anti-CD3 antibody, i.e., the ratio of CD3:CD28 is less than one. In certain embodiments of the disclosure, the ratio of anti CD28 antibody to anti CD3 antibody bound to the beads is greater than 2:1. In some embodiments, a 1:100 CD3:CD28 ratio of antibody bound to beads is used. In some embodiments, a 1:75 CD3:CD28 ratio of antibody bound to beads is used. In some embodiments, a 1:50 CD3:CD28 ratio of antibody bound to beads is used. In some embodiments, a 1:30 CD3:CD28 ratio of antibody bound to beads is used. In some embodiments, a 1:10 CD3:CD28 ratio of antibody bound to beads is used. In some embodiments, a 1:3 CD3:CD28 ratio of antibody bound to the beads is used. In yet some embodiments, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In some embodiments, a ratio of particles to cells of 1:1 or less is used. In some embodiments, a preferred particle: cell ratio is 1:5. In further embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In some embodiments, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios may be suitable for use in the present disclosure. In particular, ratios will vary depending on particle size and on cell size and type. In some embodiments, the most typical ratios for use are in the neighborhood of 1:1, 2:1 and 3:1 on the first day.

In further embodiments of the present disclosure, the cells, such as T cells, are combined with agent-coated beads, the beads and the cells are subsequently separated, and then the cells are cultured. In some embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In some embodiments, the cells (for example, 104 to 109 T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in some embodiments, a concentration of about 10 billion cells/ml, 9 billion/ml, 8 billion/ml, 7 billion/ml, 6 billion/ml, 5 billion/ml, or 2 billion cells/ml is used. In some embodiments, greater than 100 million cells/ml is used. In some embodiments, a concentration of cells of 10, 15, 20, 25, 30, 35, 40, 45, or 50 million cells/ml is used. In yet some embodiments, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, cells transduced with a nucleic acid encoding a CAR molecule, e.g., a CAR molecule described herein, are expanded, e.g., by a method described herein. In some embodiments, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, e.g., 7, 6 or 5 days. In some embodiments, the cells, e.g., a CAR-expressing cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In some embodiments, the cells, e.g., a CAR-expressing cell described herein, expanded for 5 days show at least a one, two, three or four fold increase in cells doublings upon antigen stimulation as compared to the same cells expanded in culture for 9 days under the same culture conditions. In some embodiments, the cells, e.g., the cells expressing a CAR molecule described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In some embodiments, the cells, e.g., a CAR-expressing cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

In some embodiments of the present disclosure, the mixture may be cultured for several hours (about 3 hours) to about 14 days or any hourly integer value in between. In some embodiments, the mixture may be cultured for 21 days. In some embodiments of the disclosure, the beads and the T cells are cultured together for about eight days. In some embodiments, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-1β, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In some embodiments, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In some embodiments, the cells are expanded in the presence of IL-15 and/or IL-7 (e.g., IL-15 and IL-7).

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In some embodiments, the CAR-expressing cell described herein is contacted with a composition comprising hetIL-15 during ex vivo expansion. In some embodiments, the CAR-expressing cell described herein is contacted with a composition comprising an IL-15 polypeptide during ex vivo expansion. In some embodiments, the CAR-expressing cell described herein is contacted with a composition comprising both an IL-15 polypeptide and an IL-15Ra polypeptide during ex vivo expansion. In some embodiments the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population. Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR molecule is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR molecule are described in further detail below.

Western blot analysis of CAR molecule expression in primary T cells can be used to detect the presence of monomers and dimers. See, e.g., Milone et al., Molecular Therapy 17(8): 1453-1464 (2009). Very briefly, T cells (1:1 mixture of CD4+ and CD8+ T cells) expressing the CAR molecules are expanded in vitro for more than 10 days followed by lysis and SDS-PAGE under reducing conditions. CAR molecules containing the full-length TCR-ξ cytoplasmic domain and the endogenous TCR-ξ chain are detected by western blotting using an antibody to the TCR-ξ chain. The same T cell subsets are used for SDS-PAGE analysis under non-reducing conditions to permit evaluation of covalent dimer formation.

In vitro expansion of CAR T cells following antigen stimulation can be measured by flow cytometry. For example, a mixture of CD4+ and CD8+ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with the CAR on day 1 using a multicistronic lentiviral vector expressing the CAR along with eGFP using a 2A ribosomal skipping sequence. Cultures are re-stimulated with antigen-expressing cells, such as multiple myeloma cell lines or K562 expressing the antigen, following washing. Exogenous IL-2 is added to the cultures every other day at 100 IU/ml. GFP+ T cells are enumerated by flow cytometry using bead-based counting. See, e.g., Milone et al., Molecular Therapy 17(8): 1453-1464 (2009).

Sustained CAR+ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CART activity. For example, xenograft model using human antigen-specific CAR T cells to treat a primary human multiple myeloma in immunodeficient mice can be used. See, e.g., Milone et al., Molecular Therapy 17(8): 1453-1464 (2009). Very briefly, after establishment of MM, mice are randomized as to treatment groups. Different numbers of CAR T cells can be injected into immunodeficient mice bearing MM. Animals are assessed for disease progression and tumor burden at weekly intervals. Survival curves for the groups are compared using the log-rank test. In addition, absolute peripheral blood CD4⁺ and CD8⁺ T cell counts 4 weeks following T cell injection in the immunodeficient mice can also be analyzed. Mice are injected with multiple myeloma cells and 3 weeks later are injected with T cells engineered to express a CAR molecule. T cells are normalized to 45-50% input GFP⁺ T cells by mixing with mock-transduced cells prior to injection, and confirmed by flow cytometry. Animals are assessed for leukemia at 1-week intervals. Survival curves for the CAR T cell groups are compared using the log-rank test.

Assessment of cell proliferation and cytokine production has been previously described, e.g., at Milone et al., Molecular Therapy 17(8): 1453-1464 (2009). Briefly, assessment of CAR IL-15R/IL-15-mediated proliferation is performed in microtiter plates by mixing washed T cells with K562 cells expressing the antigen or other antigen-expressing myeloma cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8⁺ T cell expansion ex vivo. T cells are enumerated in cultures using CountBright™ fluorescent beads (Invitrogen, Carlsbad, CA) and flow cytometry as described by the manufacturer. CAR T cells are identified by GFP expression using T cells that are engineered with eGFP-2A linked CAR-expressing lentiviral vectors. For CAR positive T cells not expressing GFP, the CAR+ T cells are detected with biotinylated recombinant antigen protein and a secondary avidin-PE conjugate. CD4⁺ and CD8⁺ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences, San Diego, CA) according the manufacturer's instructions. Fluorescence is assessed using a FACScalibur flow cytometer, and data is analyzed according to the manufacturer's instructions.

Cytotoxicity can be assessed by a standard 51Cr-release assay. See, e.g., Milone et al., Molecular Therapy 17(8): 1453-1464 (2009). Briefly, target cells (e.g., K562 lines expressing the antigen and primary multiple myeloma cells) are loaded with 51Cr (as NaCrO4, New England Nuclear, Boston, MA) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released 51Cr is then measured using a gamma particle counter (Packard Instrument Co., Waltham, MA). Each condition is performed in at least triplicate, and the percentage of lysis is calculated using the formula: % Lysis=(ER−SR)/(TR−SR), where ER represents the average 51Cr released for each experimental condition. Alternatively, cytotoxicity can also be assessed using a Bright-Glo™ Luciferase Assay.

Imaging technologies can be used to evaluate specific trafficking and proliferation of CAR-expressing cells in tumor-bearing animal models. Such assays have been described, for example, in Barrett et al., Human Gene Therapy 22:1575-1586 (2011). Briefly, NOD/SCID/γc^{-/-} (NSG) mice or other immunodeficient are injected IV with multiple myeloma cells followed 7 days later with CART cells 4 hour after electroporation with the CAR or CAR constructs. The T cells are stably transfected with a lentiviral construct to express firefly luciferase, and mice are imaged for bioluminescence. Alternatively, therapeutic efficacy and specificity of a single injection of CAR+ T cells in a multiple myeloma xenograft model can be measured as the following: NSG mice are injected with multiple myeloma cells transduced to stably express firefly luciferase, followed by a single tail-vein injection of T cells electroporated with CAR construct days later. Animals are imaged at various time points post injection. For example, photon-density heat maps of firefly luciferase positive tumors in representative mice at day 5 (2 days before treatment) and day 8 (24 hr post CAR+ PBLs) can be generated.

Alternatively, or in combination to the methods disclosed herein, methods and compositions for one or more of: detection and/or quantification of CAR cells (e.g., in vitro or in vivo (e.g., clinical monitoring)); immune cell expansion and/or activation; and/or CAR-specific selection, that involve the use of a CAR ligand, are disclosed. In some embodiments, the CAR ligand is an antibody that binds to the CAR molecule, e.g., binds to the extracellular antigen-binding domain of CAR (e.g., an antibody that binds to the antigen-binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In some embodiments, a method for detecting and/or quantifying CAR-expressing cells is disclosed. For example, the CAR ligand can be used to detect and/or quantify CAR cells in vitro or in vivo (e.g., clinical monitoring of CAR-expressing cells in a patient, or dosing a patient). The method includes:
providing the CAR ligand (optionally, a labelled CAR ligand, e.g., a CAR ligand that includes a tag, a bead, a radioactive or fluorescent label);
acquiring the CAR-expressing cell (e.g., acquiring a sample containing CAR cells, such as a manufacturing sample or a clinical sample);
contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In some embodiments, a method of expanding and/or activating cells (e.g., immune effector cells) is disclosed. The method includes:
providing a CAR-expressing cell (e.g., a first CAR-expressing cell or a transiently expressing CAR cell);
contacting said CAR-expressing cell with a CAR ligand, e.g., a CAR ligand as described herein), under conditions where immune cell expansion and/or proliferation occurs, thereby producing the activated and/or expanded cell population.

In some embodiments, the CAR ligand is present on (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In some embodiments, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet some embodiments, a method for selecting or enriching for a CAR-expressing cell is provided. The method includes contacting the CAR-expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR-expressing cell with a CAR ligand as described herein; and targeting the cell on the basis of binding of the CAR ligand, thereby reducing the number, and/or killing, the CAR-expressing cell. In some embodiments, the CAR ligand is coupled to a toxic agent (e.g., a toxin or a cell ablative drug). In some embodiments, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS Mar. 2013 8:3 e57838, the contents of which are incorporated by reference. In some embodiments, the anti-idiotypic antibody molecule recognizes an anti-CD19 antibody molecule, e.g., an anti-CD19 scFv. For instance, the anti-idiotypic antibody molecule can compete for binding with the CD19-specific CAR mAb clone no. 136.20.1 described in Jena et al., PLOS Mar. 2013 8:3 e57838; may have the same CDRs (e.g., one or more of, e.g., all of, VH CDR1, VH CDR2, CH CDR3, VL CDR1, VL CDR2, and VL CDR3, using the Kabat definition, the Chothia definition, or a combination of the Kabat and Chothia definitions) as the CD19-specific CAR mAb clone no. 136.20.1; may have one or more (e.g., 2) variable regions as the CD19-specific CAR mAb clone no. 136.20.1, or may comprise the CD19-specific CAR mAb clone no. 136.20.1. In some embodiments, the anti-idiotypic antibody was made according to a method described in Jena et al. In some embodiments, the anti-idiotypic antibody molecule is an anti-idiotypic antibody molecule described in WO 2014/190273. In some embodiments, the anti-idiotypic antibody molecule has the same CDRs (e.g., one or more of, e.g., all of, VH CDR1, VH CDR2, CH CDR3, VL CDR1, VL CDR2, and VL CDR3) as an antibody molecule of WO 2014/190273 such as 136.20.1; may have one or more (e.g., 2) variable regions of an antibody molecule of WO 2014/190273, or may comprise an antibody molecule of WO 2014/190273 such as 136.20.1. In other embodiments, the anti-CAR antibody binds to a constant region of the extracellular binding domain of the CAR molecule, e.g., as described in WO 2014/190273. In some embodiments, the anti-CAR antibody binds to a constant region of the extracellular binding domain of the CAR molecule, e.g., a heavy chain constant region (e.g., a CH2-CH3 hinge region) or light chain constant region. For instance, in some embodiments the anti-CAR antibody competes for binding with the 2D3 monoclonal antibody described in WO 2014/190273, has the same CDRs (e.g., one or more of, e.g., all of, VH CDR1, VH CDR2, CH CDR3, VL CDR1, VL CDR2, and VL CDR3) as 2D3, or has one or more (e.g., 2) variable regions of 2D3, or comprises 2D3 as described in WO 2014/190273.

In some embodiments, the compositions and methods herein are optimized for a specific subset of T cells, e.g., as described in U.S. Ser. No. 62/031,699 filed Jul. 31, 2014, the contents of which are incorporated herein by reference in their entirety. In some embodiments, the optimized subsets of T cells display an enhanced persistence compared to a control T cell, e.g., a T cell of a different type (e.g., CD8⁺ or CD4⁺) expressing the same construct.

In some embodiments, a CD4⁺ T cell comprises a CAR molecule described herein, which molecule CAR comprises an intracellular signaling domain suitable for (e.g., optimized for, e.g., leading to enhanced persistence in) a CD4⁺ T cell, e.g., an ICOS domain. In some embodiments, a CD8⁺ T cell comprises a CAR molecule described herein, which CAR molecule comprises an intracellular signaling domain suitable for (e.g., optimized for, e.g., leading to enhanced persistence of) a CD8⁺ T cell, e.g., a 4-1BB domain, a CD28 domain, or another costimulatory domain other than an ICOS domain.

In some embodiments, described herein is a method of treating a subject, e.g., a subject having cancer. The method includes administering to said subject, an effective amount of:
1) a CD4⁺ T cell comprising a CAR molecule (the CAR^{CD4+})
comprising:
An antigen-binding domain, e.g., an antigen-binding domain described herein;
a transmembrane domain; and
an intracellular signaling domain, e.g., a first costimulatory domain, e.g., an ICOS domain; and
2) a CD8′ T cell comprising a CAR molecule (the CAR^{CD8+}) comprising:
an antigen-binding domain, e.g., an antigen-binding domain described herein;
a transmembrane domain; and
an intracellular signaling domain, e.g., a second costimulatory domain, e.g., a 4-1BB domain, a CD28 domain, or another costimulatory domain other than an ICOS domain;
wherein the CAR^{CD4+} and the CAR^{CD8+} differ from one another.

Optionally, the method further includes administering:
3) a second CD8⁺ T cell comprising a CAR molecule (the second CAR^{CD8+}) comprising:
an antigen-binding domain, e.g., an antigen-binding domain described herein;
a transmembrane domain; and
an intracellular signaling domain, wherein the second CAR^{CD8+} comprises an intracellular signaling domain, e.g., a costimulatory signaling domain, not present on the CAR^{CD8+} and, optionally, does not comprise an ICOS signaling domain.

Other assays, including those that are known in the art can also be used to evaluate the CAR molecules of the disclosure.

Combination Therapies with CAR Cells

CAR-expressing cells described herein can be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, the CAR therapy and the additional agent (e.g., second or third agent), or all, can be administered in an amount or dose that is higher, lower or the same than the amount or dosage of each agent used individually, e.g., as a monotherapy. In some embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In some embodiments, the disclosure discloses a combination therapy including a CAR-expressing cell therapy described herein, an RNA molecule described herein (or a nucleic acid molecule encoding the RNA molecule), and an additional therapeutic agent.

### Biopolymer CAR Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic.

Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97-101; and WO2014/110591.

### Pharmaceutical Compositions and Treatments Relating to CAR Cells

Pharmaceutical compositions of the present disclosure can comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in some embodiments formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, the pharmaceutical composition is substantially free of, e.g., there are no detectable levels of a contaminant, e.g., selected from the group consisting of endotoxin, mycoplasma, replication competent lentivirus (RCL), β₂₄, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 104 to 10⁹ cells/kg body weight, in some instances 105 to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain embodiments, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In some embodiments, the T cell compositions of the present disclosure are administered to a patient by intradermal or subcutaneous injection. In some embodiments, the CAR-expressing cell (e.g., T cell or NK cell) compositions of the present disclosure are administered by i.v. injection. The compositions of CAR-expressing cells (e.g., T cells or NK cells) may be injected directly into a tumor, lymph node, or site of infection.

In some embodiments, subjects may undergo leukapheresis, wherein leukocytes are collected, enriched, or depleted ex vivo to select and/or isolate the cells of interest, e.g., immune effector cells (e.g., T cells or NK cells). These immune effector cell (e.g., T cell or NK cell) isolates may be expanded by methods known in the art and treated such that one or more CAR constructs of the disclosure may be introduced, thereby creating a CAR-expressing cell (e.g., CAR T cell or CAR-expressing NK cell) of the disclosure. Subjects in need thereof may subsequently undergo standard treatment with high dose chemotherapy followed by peripheral blood stem cell transplantation. In certain embodiments, following or concurrent with the transplant, subjects receive an infusion of the expanded CAR-expressing cells (e.g., CAR T cells or NK cells) of the present disclosure. In some embodiments, expanded cells are administered before or following surgery.

In embodiments, lymphodepletion is performed on a subject, e.g., prior to administering one or more cells that express a CAR molecule described herein. In embodiments, the lymphodepletion comprises administering one or more of melphalan, cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In some embodiments, the CAR molecule is introduced into immune effector cells (e.g., T cells or NK cells), e.g., using in vitro transcription, and the subject (e.g., human) receives an initial administration of CAR immune effector cells (e.g., T cells or NK cells) of the disclosure, and one or more subsequent administrations of the CAR immune effector cells (e.g., T cells or NK cells) of the disclosure, wherein the one or more subsequent administrations are administered less than 15 days, e.g., 14, 13, 12, 11, 10, 9, 8, 7, 6, 5, 4, 3, or 2 days after the previous administration. In some embodiments, more than one administration of the CAR immune effector cells (e.g., T cells or NK cells) of the disclosure are administered to the subject (e.g., human) per week, e.g., 2, 3, or 4 administrations of the CAR immune effector cells (e.g., T cells or NK cells) of the disclosure are administered per week. In some embodiments, the subject (e.g., human subject) receives more than one administration of the CAR immune effector cells (e.g., T cells or NK cells) per week (e.g., 2, 3 or 4 administrations per week) (also referred to herein as a cycle), followed by a week of no CAR immune effector cells (e.g., T cells or NK cells) administrations, and then one or more additional administration of the CAR immune effector cells (e.g., T cells or NK cells) (e.g., more than one administration of the CAR immune effector cells (e.g., T cells or NK cells) per week) is administered to the subject. In some embodiments, the subject (e.g., human subject) receives more than one cycle of CAR immune effector cells (e.g., T cells or NK cells), and the time between each cycle is less than 10, 9, 8, 7, 6, 5, 4, or 3 days. In some embodiments, the CAR immune effector cells (e.g., T cells or NK cells) are administered every other day for 3 administrations per week. In some embodiments, the CAR immune effector cells (e.g., T cells or NK cells) of the disclosure are administered for at least two, three, four, five, six, seven, eight or more weeks.

In some embodiments, CAR-expressing cells (e.g., CARTs or CAR-expressing NK cells) are generated using lentiviral viral vectors, such as lentivirus. CAR-expressing cells (e.g., CARTs or CAR-expressing NK cells) generated that way will have stable CAR expression.

In some embodiments, CAR-expressing cells, e.g., CARTs, are generated using a viral vector such as a gammaretroviral vector, e.g., a gammaretroviral vector described herein. CARTs generated using these vectors can have stable CAR expression.

In some embodiments, CAR-expressing cells (e.g., CARTs or CAR-expressing NK cells) transiently express CAR vectors for 4, 5, 6, 7, 8, 9, 10, 11, 12, 13, 14, 15 days after transduction. Transient expression of CAR molecules can be effected by RNA CAR vector delivery. In some embodiments, the CAR RNA is transduced into the cell, e.g., T cell or NK cell, by electroporation.

A potential issue that can arise in patients being treated using transiently expressing CAR-expressing cells (e.g., CARTs or CAR-expressing NK cells) (particularly with murine scFv bearing CAR-expressing cells (e.g., CARTs or CAR-expressing NK cells)) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell (e.g., CART or CAR-expressing NK cell) infusion breaks should not last more than ten to fourteen days.

### Methods Using Biomarkers for Evaluating CAR-Effectiveness, Subject Suitability, or Sample Suitability

In some embodiments, the disclosure features a method of evaluating or monitoring the effectiveness of a CAR-expressing cell therapy in a subject (e.g., a subject having a cancer). The method includes acquiring a value of effectiveness to the CAR therapy, subject suitability, or sample suitability, wherein said value is indicative of the effectiveness or suitability of the CAR-expressing cell therapy.

In some embodiments of any of the methods disclosed herein, the subject is evaluated prior to receiving, during, or after receiving, the CAR-expressing cell therapy.

In some embodiments of any of the methods disclosed herein, a responder (e.g., a complete responder) has, or is identified as having, a greater level or activity of one, two, or more (all) of GZMK, PPF1BP2, or naïve T cells as compared to a non-responder.

In some embodiments of any of the methods disclosed herein, a non-responder has, or is identified as having, a greater level or activity of one, two, three, four, five, six, seven, or more (e.g., all) of IL22, IL-2RA, IL-21, IRF8, IL8, CCL17, CCL22, effector T cells, or regulatory T cells, as compared to a responder.

In some embodiments, a relapser is a patient having, or who is identified as having, an increased level of expression of one or more of (e.g., 2, 3, 4, or all of) the following genes, compared to non relapsers: MIR199A1, MIR1203, uc02lovp, ITM2C, and HLA-DQB1 and/or a decreased levels of expression of one or more of (e.g., 2, 3, 4, 5, 6, 7, 8, 9, 10, 11, or all of) the following genes, compared to non relapsers: PPIAL4D, TTTY10, TXLNG2P, MIR4650-1, KDM5D, USP9Y, PRKY, RPS4Y2, RPS4Y1, NCRNA00185, SULT1E1, and EIF1AY.

In some embodiments of any of the methods disclosed herein, a non-responder has, or is identified as having, a greater percentage of an immune cell exhaustion marker, e.g., one, two or more immune checkpoint inhibitors (e.g., PD-1, PD-L1, TIM-3 and/or LAG-3). In some embodiments, a non-responder has, or is identified as having, a greater percentage of PD-1, PD-L1, or LAG-3 expressing immune effector cells (e.g., CD4+ T cells and/or CD8+ T cells) (e.g., CAR-expressing CD4+ cells and/or CD8+ T cells) compared to the percentage of PD-1 or LAG-3 expressing immune effector cells from a responder.

In some embodiments, a non-responder has, or is identified as having, a greater percentage of immune cells having an exhausted phenotype, e.g., immune cells that co-express at least two exhaustion markers, e.g., co-expresses PD-1, PD-L1 and/or TIM-3. In other embodiments, a non-responder has, or is identified as having, a greater percentage of immune cells having an exhausted phenotype, e.g., immune cells that co-express at least two exhaustion markers, e.g., co-expresses PD-1 and LAG-3.

In some embodiments of any of the methods disclosed herein, a non-responder has, or is identified as having, a greater percentage of PD-1/PD-L1⁺/LAG-3⁺ cells in the CARexpressing cell population compared to a responder (e.g., a complete responder) to the CAR-expressing cell therapy.

In some embodiments of any of the methods disclosed herein, a partial responder has, or is identified as having, a higher percentages of PD-1/PD-L1⁺/LAG-3⁺ cells, than a responder, in the CAR-expressing cell population.

In some embodiments of any of the methods disclosed herein, a non-responder has, or is identified as having, an exhausted phenotype of PD1/PD-L1⁺ CAR+ and co-expression of LAG3 in the CAR-expressing cell population.

In some embodiments of any of the methods disclosed herein, a non-responder has, or is identified as having, a greater percentage of PD-1/PD-L1⁺/TIM-3⁺ cells in the CAR-expressing cell population compared to the responder (e.g., a complete responder).

In some embodiments of any of the methods disclosed herein, a partial responders has, or is identified as having, a higher percentage of PD-1/PD-L1⁺/TIM-3⁺ cells, than responders, in the CAR-expressing cell population.

In some embodiments of any of the methods disclosed herein, the presence of CD8⁺ CD27⁺ CD45RO− T cells in an apheresis sample is a positive predictor of the subject response to a CAR-expressing cell therapy.

In some embodiments of any of the methods disclosed herein, a high percentage of PD1⁺ CAR+ and LAG3⁺ or TIM3⁺ T cells in an apheresis sample is a poor prognostic predictor of the subject response to a CAR-expressing cell therapy.

In some embodiments of any of the methods disclosed herein, the responder (e.g., the complete or partial responder) has one, two, three or more (or all) of the following profile:
(i) has a greater number of CD27⁺μmmune effector cells compared to a reference value, e.g., a non-responder number of CD27⁺μmmune effector cells;
(ii) has a greater number of CD8⁺ T cells compared to a reference value, e.g., a non-responder number of CD8⁺ T cells;
(iii) has a lower number of immune cells expressing one or more checkpoint inhibitors, e.g., a checkpoint inhibitor chosen from PD-1, PD-L1, LAG-3, TIM-3, or KLRG-1, or a combination, compared to a reference value, e.g., a non-responder number of cells expressing one or more checkpoint inhibitors; or
(iv) has a greater number of one, two, three, four or more (all) of resting T_{EFF} cells, resting T_{REG} cells, naïve CD4 cells, unstimulated memory cells or early memory T cells, or a combination thereof, compared to a reference value, e.g., a non-responder number of resting T_{EFF} cells, resting T_{REG} cells, naïve CD4 cells, unstimulated memory cells or early memory T cells.

In some embodiments of any of the methods disclosed herein, the cytokine level or activity is chosen from one, two, three, four, five, six, seven, eight, or more (or all) of cytokine CCL20/MIP3a, IL17A, IL6, GM-CSF, IFN-γ, IL10, IL13, IL2, IL21, IL4, IL5, IL9 or TNFα, or a combination thereof. The cytokine can be chosen from one, two, three, four or more (all) of IL-17a, CCL20, IL2, IL6, or TNFα. In some embodiments, an increased level or activity of a cytokine is chosen from one or both of IL-17a and CCL20, is indicative of increased responsiveness or decreased relapse. In embodiments, the responder, a non-responder, a relapser or a non-relapser identified by the methods herein can be further evaluated according to clinical criteria. For example, a complete responder has, or is identified as, a subject having a disease, e.g., a cancer, who exhibits a complete response, e.g., a complete remission, to a treatment. A complete response may be identified, e.g., using the NCCN Guidelines®, or Cheson et al, J Clin Oncol 17:1244 (1999) and Cheson et al., “Revised Response Criteria for Malignant Lymphoma”, J Clin Oncol 25:579-586 (2007) (both of which are incorporated by reference herein in their entireties), as described herein. A partial responder has, or is identified as, a subject having a disease, e.g., a cancer, who exhibits a partial response, e.g., a partial remission, to a treatment. A partial response may be identified, e.g., using the NCCN Guidelines®, or Cheson criteria as described herein. A non-responder has, or is identified as, a subject having a disease, e.g., a cancer, who does not exhibit a response to a treatment, e.g., the patient has stable disease or progressive disease. A non-responder may be identified, e.g., using the NCCN Guidelines®, or Cheson criteria as described herein.

Alternatively, or in combination with the methods disclosed herein, responsive to said value, performing one, two, three, four or more of:
administering e.g., to a responder or a non-relapser, a CAR-expressing cell therapy;
administered an altered dosing of a CAR-expressing cell therapy;
altering the schedule or time course of a CAR-expressing cell therapy;
administering, e.g., to a non-responder or a partial responder, an additional agent in combination with a CAR-expressing cell therapy, e.g., a checkpoint inhibitor, e.g., a checkpoint inhibitor described herein;
administering to a non-responder or partial responder a therapy that increases the number of younger T cells in the subject prior to treatment with a CAR-expressing cell therapy;
modifying a manufacturing process of a CAR-expressing cell therapy, e.g., enriching for younger T cells prior to introducing a nucleic acid encoding a CAR molecule, or increasing the transduction efficiency, e.g., for a subject identified as a non-responder or a partial responder;
administering an alternative therapy, e.g., for a non-responder or partial responder or relapser; or
if the subject is, or is identified as, a non-responder or a relapser, decreasing the T_{REG} cell population and/or T_{REG} gene signature, e.g., by one or more of CD25 depletion, administration of cyclophosphamide, anti-GITR antibody, or a combination thereof.

In some embodiments, the subject is pre-treated with an anti-GITR antibody. In some embodiments, the subject is treated with an anti-GITR antibody prior to infusion or re-infusion.

### Method of Treating Cancer Using ZBTB32 Inhibitors

In one aspect, the disclosure relates to treatment of a subject in vivo using a ZBTB32 inhibitor (e.g., a ZBTB32 inhibitor described herein), alone or in combination with a second therapeutic agent or modality (e.g., a therapeutic agent or modality disclosed herein), or a composition or formulation comprising a combination disclosed herein, such that growth of cancerous tumors is inhibited or reduced.

In one embodiment, the ZBTB32 inhibitor or combination disclosed herein is suitable for the treatment of cancer in vivo. For example, the ZBTB32 inhibitor or combination can be used to inhibit the growth of cancerous tumors. The ZBTB32 inhibitor or combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest. A combination disclosed herein can be administered in either order or simultaneously.

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject a ZBTB32 inhibitor (e.g., a ZBTB32 inhibitor described herein), alone or in combination with a second therapeutic agent or modality (e.g., a therapeutic agent or modality disclosed herein), or a composition or formulation comprising a combination disclosed herein, e.g., in accordance with a dosage regimen disclosed herein.

As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial, bladder cells), prostate, CNS (e.g., brain, neural or glial cells), skin, pancreas, and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. Metastatic lesions of the aforementioned cancers can also be treated or prevented using the methods and compositions of the disclosure.

In some embodiments, the cancer is chosen from a breast cancer, a pancreatic cancer, a colorectal cancer, a skin cancer, a gastric cancer, or an ER+ cancer. In some embodiments, the skin cancer is a melanoma (e.g., a refractory melanoma). In some embodiments, the ER+ cancer is an ER+ breast cancer. In some embodiments, the cancer is an Epstein Barr Virus (EBV) positive cancer.

Exemplary cancers whose growth can be inhibited using the combinations disclosed herein, include cancers typically responsive to immunotherapy. Non-limiting examples of typical cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the antibody molecules described herein.

Examples of other cancers that can be treated include, but are not limited to, basal cell carcinoma, biliary tract cancer; bladder cancer; bone cancer; brain and CNS cancer; primary CNS lymphoma; neoplasm of the central nervous system (CNS); breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia (including acute myeloid leukemia, chronic myeloid leukemia, acute lymphoblastic leukemia, chronic lymphocytic leukemia, chronic or acute leukemia); liver cancer; lung cancer (e.g., small cell and non-small cell); lymphoma including Hodgkin's and non-Hodgkin's lymphoma; lymphocytic lymphoma; melanoma, e.g., cutaneous or intraocular malignant melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, hepatocarcinoma, cancer of the anal region, carcinoma of the fallopian tubes, carcinoma of the vagina, carcinoma of the vulva, cancer of the small intestine, cancer of the endocrine system, cancer of the parathyroid gland, cancer of the adrenal gland, sarcoma of soft tissue, cancer of the urethra, cancer of the penis, solid tumors of childhood, spinal axis tumor, brain stem glioma, pituitary adenoma, Kaposi's sarcoma, epidermoid cancer, squamous cell cancer, T-cell lymphoma, environmentally induced cancers including those induced by asbestos, as well as other carcinomas and sarcomas, and combinations of said cancers.

In some embodiments, the disorder is a cancer, e.g., a cancer described herein. In certain embodiments, the cancer is a solid tumor. In some embodiments, the cancer is brain tumor, e.g., a glioblastoma, a gliosarcoma, or a recurrent brain tumor. In some embodiments, the cancer is a pancreatic cancer, e.g., an advanced pancreatic cancer. In some embodiments, the cancer is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, the cancer is a renal cancer, e.g., a renal cell carcinoma (RCC) (e.g., a metastatic renal cell carcinoma) or a treatment-naïve metastatic kidney cancer. In some embodiments, the cancer is a breast cancer, e.g., a metastatic breast carcinoma or a stage IV breast carcinoma, e.g., a triple negative breast cancer (TNBC). In some embodiments, the cancer is a virus-associated cancer. In some embodiments, the cancer is an anal canal cancer (e.g., a squamous cell carcinoma of the anal canal). In some embodiments, the cancer is a cervical cancer (e.g., a squamous cell carcinoma of the cervix). In some embodiments, the cancer is a gastric cancer (e.g., an Epstein Barr Virus (EBV) positive gastric cancer, or a gastric or gastro-esophageal junction carcinoma). In some embodiments, the cancer is a head and neck cancer (e.g., an HPV positive and negative squamous cell cancer of the head and neck (SCCHN)). In some embodiments, the cancer is a nasopharyngeal cancer (NPC). In some embodiments, the cancer is a penile cancer (e.g., a squamous cell carcinoma of the penile). In some embodiments, the cancer is a vaginal or vulvar cancer (e.g., a squamous cell carcinoma of the vagina or vulva). In some embodiments, the cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer. In some embodiments, the cancer is a lung cancer, e.g., a non-small cell lung cancer (NSCLC). In certain embodiments, the cancer is a hematological cancer. In some embodiments, the cancer is a leukemia. In some embodiments, the cancer is a lymphoma, e.g., a Hodgkin lymphoma (HL) or a diffuse large B cell lymphoma (DLBCL) (e.g., a relapsed or refractory HL or DLBCL). In some embodiments, the cancer is a myeloma. In some embodiments, the cancer is an MSI-high (MSI-H) cancer. In some embodiments, the cancer is a metastatic cancer. In other embodiments, the cancer is an advanced cancer. In other embodiments, the cancer is a relapsed or refractory cancer.

In one embodiment, the cancer is a Merkel cell carcinoma. In other embodiments, the cancer is a melanoma. In other embodiments, the cancer is a breast cancer, e.g., a triple negative breast cancer (TNBC) or a HER2-negative breast cancer. In other embodiments, the cancer is a renal cell carcinoma (e.g., a clear cell renal cell carcinoma (CCRCC) or a non-clear cell renal cell carcinoma (nccRCC)). In other embodiments, the cancer is a thyroid cancer, e.g., an anaplastic thyroid carcinoma (ATC). In other embodiments, the cancer is a neuroendocrine tumor (NET), e.g., an atypical pulmonary carcinoid tumor or a NET in pancreas, gastrointestinal (GI) tract, or lung. In certain embodiments, the cancer is a non-small cell lung cancer (NSCLC) (e.g., a squamous NSCLC or a non-squamous NSCLC). In certain embodiments, the cancer is a fallopian tube cancer. In certain embodiments, the cancer is a microsatellite instability-high colorectal cancer (MSI-high CRC) or a microsatellite stable colorectal cancer (MSS CRC).

In other embodiments, the cancer is a hematological malignancy or cancer including but is not limited to a leukemia or a lymphoma. For example, the combination can be used to treat cancers and malignancies including, but not limited to, e.g., an acute leukemia, e.g., B-cell acute lymphoid leukemia (“BALL”), T-cell acute lymphoid leukemia (“TALL”), acute lymphoid leukemia (ALL); a chronic leukemia, e.g., chronic myelogenous leukemia (CML), chronic lymphocytic leukemia (CLL); an additional hematologic cancer or hematologic condition, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like.

As used herein, the term “subject” is intended to include human and non-human animals. In some embodiments, the subject is a human subject, e.g., a human patient having a disorder or condition characterized by abnormal immune checkpoint functioning. For example, the subject has at least some PD-1 protein, including the PD-1 epitope that is bound by an anti-PD-1 antibody molecule, e.g., a high enough level of the protein and epitope to support antibody binding to PD-1. The term “non-human animals” includes mammals and non-mammals, such as non-human primates. In some embodiments, the subject is a human. In some embodiments, the subject is a human patient in need of enhancement of an immune response. The methods and compositions described herein are suitable for treating human patients having a disorder that can be treated by modulating (e.g., augmenting or inhibiting) an immune response. In certain embodiments, the cancer is a cancer for which the immune response is deficient or an immunogenic cancer.

Methods and compositions disclosed herein are useful for treating metastatic lesions associated with the aforementioned cancers.

In some embodiments, the method further comprises determining whether a tumor sample is positive for one or more of PD-L1, CD8, and IFN-γ, and if the tumor sample is positive for one or more, e.g., two, or all three, of the markers, then administering to the patient a therapeutically effective amount of a combination of therapeutic agents, as described herein.

In some embodiments, the ZBTB32 inhibitor or combination is used to treat a cancer that expresses one or more of the biomarkers disclosed herein. In certain embodiments, the subject or cancer is treated responsive to the determination of the presence of one or more biomarkers disclosed herein.

In other embodiments, the ZBTB32 inhibitor or combination is used to treat a cancer that is characterized by microsatellite instability-high (MSI-H) or mismatch repair deficient (dMMR). The identification of MSI-H or dMMR tumor status for patients can be determined using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; Kawakami et al. Curr Treat Options Oncol. 2015; 16(7): 30).

The combination therapies described herein can include a composition of the present disclosure co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

When administered in combination, the therapeutic agent can be administered in an amount or dose that is higher or lower than, or the same as, the amount or dosage of each agent used individually, e.g., as a monotherapy. In certain embodiments, the administered amount or dosage of the therapeutic agent is lower (e.g., at least 20%, at least 30%, at least 40%, or at least 50%) than the amount or dosage of each agent used individually, e.g., as a monotherapy. In other embodiments, the amount or dosage of the therapeutic agent that results in a desired effect (e.g., treatment of cancer) is lower (e.g., at least 20%, at least 30%, at least 40%, or at least 50% lower).

Combination Therapy with ZBTB32 Inhibitors

The ZBTB32 inhibitors of the disclosure can be administered in therapeutically effective amounts in a combinational therapy with one or more therapeutic agents (pharmaceutical combinations) or modalities, e.g., non-drug therapies. For example, synergistic effects can occur with other cancer agents. Where the ZBTB32 inhibitors of the disclosure are administered in conjunction with other therapies, dosages of the co-administered ZBTB32 inhibitors will of course vary depending on the type of co-drug employed, on the specific drug employed, on the condition being treated and so forth.

The ZBTB32 inhibitors can be administered simultaneously (as a single preparation or separate preparation), sequentially, separately, or over a period of time to the other drug therapy or treatment modality. In general, a combination therapy envisions administration of two or more drugs during a single cycle or course of therapy. A therapeutic agent is, for example, a chemical compound, peptide, antibody, antibody fragment or nucleic acid, which is therapeutically active or enhances the therapeutic activity when administered to a patient in combination with a compound of the present disclosure.

In one aspect, a ZBTB32 inhibitor, or a pharmaceutically acceptable salt, hydrate, solvate, prodrug, stereoisomer, or tautomer thereof, of the present disclosure can be combined with other therapeutic agents, such as other anti-cancer agents, anti-allergic agents, anti-nausea agents (or anti-emetics), pain relievers, cytoprotective agents, and combinations thereof.

In some embodiments, the ZBTB32 inhibitor, or a pharmaceutically acceptable salt, hydrate, solvate, prodrug, stereoisomer, or tautomer thereof of the present disclosure are administered in combination with one or more second agent(s) selected from a PD-1 inhibitor, a PD-L1 inhibitor, a LAG-3 inhibitor, a cytokine, an A2aR antagonist, a GITR agonist, a TIM-3 inhibitor, a STING agonist, a CTLA-4 inhibitor, a TIGIT inhibitor, a chimeric antigen receptor, an estrogen receptor antagonist, a CDK4/6 inhibitor, a CXCR2 inhibitor, a CSF-1/1R binding agent, an IDO inhibitor, a Galectin inhibitor, a MEK inhibitor, a c-MET inhibitor, a TGF-b inhibitor, an IL-1b inhibitor, an MDM2 inhibitor, and a TLR7 agonist, to treat a disease, e.g., cancer.

In some embodiments, the ZBTB32 inhibitor is used in combination with an agonist of a costimulatory molecule chosen from one or more of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In some embodiments, the ZBTB32 inhibitor is used in combination with an inhibitor of an immune checkpoint molecule chosen from one or more of PD-L1, PD-L2, CTLA-4, TIM-3, LAG-3, CEACAM-1, CEACAM-5, VISTA, BTLA, TIGIT, LAIR1, CD160, 2B4 or TGFβ.

In another embodiment, one or more chemotherapeutic agents are used in combination with the ZBTB32 inhibitor, or a pharmaceutically acceptable salt, hydrate, solvate, prodrug, stereoisomer, or tautomer thereof, for treating a disease, e.g., cancer, wherein said chemotherapeutic agents include, but are not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® orNeosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), epirubicin (Ellence®), oxaliplatin (Eloxatin®), exemestane (Aromasin®), letrozole (Femara®), and fulvestrant (Faslodex®).

In other embodiments, the ZBTB32 inhibitor, or a pharmaceutically acceptable salt, hydrate, solvate, prodrug, stereoisomer, or tautomer thereof, of the present disclosure are used in combination with one or more other anti-HER2 antibodies, e.g., trastuzumab, pertuzumab, margetuximab, or HT-19 described above, or with other anti-HER2 conjugates, e.g., ado-trastuzumab emtansine (also known as Kadcyla®, or T-DM1).

In other embodiments, the ZBTB32 inhibitors, or a pharmaceutically acceptable salt, hydrate, solvate, prodrug, stereoisomer, or tautomer thereof, of the present disclosure are used in combination with one or more tyrosine kinase inhibitors, including but not limited to, EGFR inhibitors, Her3 inhibitors, IGFR inhibitors, and Met inhibitors, for treating a disease, e.g., cancer.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (Gilotrif®); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aa,5 p,6aa)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R3; MDX0447 (CAS 339151-96-1); and ch806 (mAb-806, CAS 946414-09-1).

Other HER2 inhibitors include but are not limited to, Neratinib (HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aa,5 p,6aa)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1Hpyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin 3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGFR inhibitors include but are not limited to, BMS-754807, XL-228, OSI-906, GSK0904529A, A-928605, AXL1717, KW-2450, MK0646, AMG479, IMCA12, MEDI-573, and B1836845. See e.g., Yee, JNCI, 104; 975 (2012) for review.

In another embodiment, the ZBTB32 inhibitor of the present disclosure are used in combination with one or more proliferation signalling pathway inhibitors, including but not limited to, MEK inhibitors, BRAF inhibitors, PI3K/Akt inhibitors, SHP2 inhibitors, and also mTOR inhibitors, and CDK inhibitors, for treating a disease, e.g., cancer.

For example, mitogen-activated protein kinase (MEK) inhibitors include but are not limited to, XL-518 (also known as GDC-0973, CAS No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

BRAF inhibitors include, but are not limited to, Vemurafenib (or Zelboraf®, PLX-4032, CAS 918504-65-1), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®).

Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC0941, RG7321, GNE0941, Pictrelisib, or Pictilisib; and described in PCT Publication Nos. WO 09/036082 and WO 09/055730); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethylcyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); (S)—N1-(4-methyl-5-(2-(1,1,1-trifluoro-2-methylpropan-2-yl)pyridin-4-yl)thiazol-2-yl)pyrrolidine-1,2-dicarboxamide (also known as BYL719 or Alpelisib); 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide (also known as GDCO0032, RG7604, or Taselisib).

mTOR inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9] hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 3201), inner salt (SF1126, CAS 936487-67-1).

CDK inhibitors include but are not limited to, Palbociclib (also known as PD-0332991, Ibrance®, 6-Acetyl-8-cyclopentyl-5-methyl-2-{[5-(1-piperazinyl)-2-pyridinyl]amino}pyrido[2,3-d]pyrimidin-7(8H)-one).

In yet another embodiment, the ZBTB32 inhibitor, or a pharmaceutically acceptable salt, hydrate, solvate, prodrug, stereoisomer, or tautomer thereof, of the present disclosure are used in combination with one or more pro-apoptotics, including but not limited to, IAP inhibitors, BCL2 inhibitors, MCL1 inhibitors, TRAIL agents, CHK inhibitors, for treating a disease, e.g., cancer.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679, all of which are incorporated herein by reference.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab(CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinylpyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL (SEQ ID NO: 33)), and CBP501 ((d-Bpa)sws(d-Phe-F5)(d-Cha)rrrqrr).

In a further embodiment, the ZBTB32 inhibitor, or a pharmaceutically acceptable salt, hydrate, solvate, prodrug, stereoisomer, or tautomer thereof, of the present disclosure are used in combination with one or more immunomodulators (e.g., one or more of an activator of a costimulatory molecule or an inhibitor of an immune checkpoint molecule), for treating a disease, e.g., cancer.

In certain embodiments, the immunomodulator is an activator of a costimulatory molecule. In one embodiment, the agonist of the costimulatory molecule is selected from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

### GITR Agonists

In some embodiments, a GITR agonist is used in combination with a ZBTB32 inhibitor, or a pharmaceutically acceptable salt, hydrate, solvate, prodrug, stereoisomer, or tautomer thereof, for treating a disease, e.g., cancer. In some embodiments, the GITR agonist is GWN323 (Novartis), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 12 (e.g., from the heavy and light chain variable region sequences of MAB7 disclosed in Table 12), or encoded by a nucleotide sequence shown in Table 12. In some embodiments, the CDRs are according to the Kabat definition (e.g., as set out in Table 12). In some embodiments, the CDRs are according to the Chothia definition (e.g., as set out in Table 12). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 12, or encoded by a nucleotide sequence shown in Table 12.

In one embodiment, the anti-GITR antibody molecule comprises a heavy chain variable region (VH) comprising a VHCDR1 amino acid sequence of SEQ ID NO: 9, a VHCDR2 amino acid sequence of SEQ ID NO: 11, and a VHCDR3 amino acid sequence of SEQ ID NO: 13; and a light chain variable region (VL) comprising a VLCDR1 amino acid sequence of SEQ ID NO: 3016, a VLCDR2 amino acid sequence of SEQ ID NO: 16, and a VLCDR3 amino acid sequence of SEQ ID NO: 18, each disclosed in Table 12.

In one embodiment, the anti-GITR antibody molecule comprises a VH comprising the amino acid sequence of SEQ ID NO: 1, or an amino acid sequence at least 85%, 90%, 95%, or 99% identical or higher to SEQ ID NO: 1. In one embodiment, the anti-GITR antibody molecule comprises a VL comprising the amino acid sequence of SEQ ID NO: 2, or an amino acid sequence at least 85%, 90%, 95%, or 99% identical or higher to SEQ ID NO: 2. In one embodiment, the anti-GITR antibody molecule comprises a VH comprising the amino acid sequence of SEQ ID NO: 1 and a VL comprising the amino acid sequence of SEQ ID NO: 2.

In one embodiment, the antibody molecule comprises a VH encoded by the nucleotide sequence of SEQ ID NO: 5, or a nucleotide sequence at least 85%, 90%, 95%, or 99% identical or higher to SEQ ID NO: 5. In one embodiment, the antibody molecule comprises a VL encoded by the nucleotide sequence of SEQ ID NO: 6, or a nucleotide sequence at least 85%, 90%, 95%, or 99% identical or higher to SEQ ID NO: 6. In one embodiment, the antibody molecule comprises a VH encoded by the nucleotide sequence of SEQ ID NO: 5 and a VL encoded by the nucleotide sequence of SEQ ID NO: 6.

In one embodiment, the anti-GITR antibody molecule comprises a heavy chain comprising the amino acid sequence of SEQ ID NO: 3, or an amino acid sequence at least 85%, 90%, 95%, or 99% identical or higher to SEQ ID NO: 3. In one embodiment, the anti-GITR antibody molecule comprises a light chain comprising the amino acid sequence of SEQ ID NO: 4, or an amino acid sequence at least 85%, 90%, 95%, or 99% identical or higher to SEQ ID NO: 4. In one embodiment, the anti-GITR antibody molecule comprises a heavy chain comprising the amino acid sequence of SEQ ID NO: 3 and a light chain comprising the amino acid sequence of SEQ ID NO: 4.

In one embodiment, the antibody molecule comprises a heavy chain encoded by the nucleotide sequence of SEQ ID NO: 7, or a nucleotide sequence at least 85%, 90%, 95%, or 99% identical or higher to SEQ ID NO: 7. In one embodiment, the antibody molecule comprises a light chain encoded by the nucleotide sequence of SEQ ID NO: 8, or a nucleotide sequence at least 8500,9000,9500, or 99% identical or higher to SEQ ID NO 8 In one embodiment, the antibody molecule comprises a heavy chain encoded bythe nucleotide sequence ofSEQ IDNO: 7 and alight chain encoded bythe nucleotide sequence of SEQ ID NO: 8.

The antibody molecules described herein can be made by vectors, host cells, and methods described in WO 2016/057846, incorporated by reference in its entirety.

TABLE 12
Amino acid and nucleotide sequences of exemplary
anti-GITR antibody molecule

| MAB7 | | |
|---|---|---|
| SEQ ID NO: 1 | VH | EVQLVESGGGLVQSGGSLRLSCAASGFSLSSYGVDWVRQAPGKG |
| | | LEWVGVIWGGGGTYYASSLMGRFTISRDNSKNTLYLQMNSLRAE |
| | | DTAVYYCARHAYGHDGGFAMDYWGQGTLVTVSS |
| SEQ ID NO: 2 | VL | EIVMTQSPATLSVSPGERATLSCRASESVSSNVAWYQQRPGQAPR |
| | | LLIYGASNRATGIPARFSGSGSGTDFTLTISRLEPEDFAVYYCGQSY |
| | | SYPFTFGQGTKLEIK |
| SEQ ID NO: 3 | Heavy | EVQLVESGGGLVQSGGSLRLSCAASGFSLSSYGVDWVRQAPGKG |
| | Chain | LEWVGVIWGGGGTYYASSLMGRFTISRDNSKNTLYLQMNSLRAE |
| | | DTAVYYCARHAYGHDGGFAMDYWGQGTLVTVSSASTKGPSVFP |
| | | LAPSSKSTSGGTAALGCLVKDYFPEPVTVSWNSGALTSGVHTFPA |
| | | VLQSSGLYSLSSVVTVPSSSLGTQTYICNVNHKPSNTKVDKRVEP |
| | | KSCDKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVYT |
| | | LPPSREEMTKNQVSLTCLVKGFYPSDIAVEWESNGQPENNYKTTP |
| | | PVLDSDGSFFLYSKLTVDKSRWQQGNVFSCSVMHEALHNHYTQK |
| | | SLSLSPGK |
| SEQ ID NO: 4 | Light | EIVMTQSPATLSVSPGERATLSCRASESVSSNVAWYQQRPGQAPR |
| | Chain | LLIYGASNRATGIPARFSGSGSGTDFTLTISRLEPEDFAVYYCGQSY |
| | | SYPFTFGQGTKLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |
| SEQ ID NO: 5 | DNA | GAGGTGCAGCTGGTGGAATCTGGCGGCGGACTGGTGCAGTCCG |
| | VH | GCGGCTCTCTGAGACTGTCTTGCGCTGCCTCCGGCTTCTCCCTG |
| | | TCCTCTTACGGCGTGGACTGGGTGCGACAGGCCCCTGGCAAGG |
| | | GCCTGGAATGGGTGGGAGTGATCTGGGGCGGAGGCGGCACCT |
| | | ACTACGCCTCTTCCCTGATGGGCCGGTTCACCATCTCCCGGGAC |
| | | AACTCCAAGAACACCCTGTACCTGCAGATGAACTCCCTGCGGG |
| | | CCGAGGACACCGCCGTGTACTACTGCGCCAGACACGCCTACGG |
| | | CCACGACGGCGGCTTCGCCATGGATTATTGGGGCCAGGGCACC |
| | | CTGGTGACAGTGTCCTCC |
| SEQ ID NO: 6 | DNA | GAGATCGTGATGACCCAGTCCCCCGCCACCCTGTCTGTGTCTCC |
| | VL | CGGCGAGAGAGCCACCCTGAGCTGCAGAGCCTCCGAGTCCGTG |
| | | TCCTCCAACGTGGCCTGGTATCAGCAGAGACCTGGTCAGGCCC |
| | | CTCGGCTGCTGATCTACGGCGCCTCTAACCGGGCCACCGGCAT |
| | | CCCTGCCAGATTCTCCGGCTCCGGCAGCGGCACCGACTTCACC |
| | | CTGACCATCTCCCGGCTGGAACCCGAGGACTTCGCCGTGTACT |
| | | ACTGCGGCCAGTCCTACTCATACCCCTTCACCTTCGGCCAGGG |
| | | CACCAAGCTGGAAATCAAG |
| SEQ ID NO: 7 | DNA | GAGGTGCAGCTGGTGGAATCTGGCGGCGGACTGGTGCAGTCCG |
| | Heavy | GCGGCTCTCTGAGACTGTCTTGCGCTGCCTCCGGCTTCTCCCTG |
| | Chain | TCCTCTTACGGCGTGGACTGGGTGCGACAGGCCCCTGGCAAGG |
| | | GCCTGGAATGGGTGGGAGTGATCTGGGGCGGAGGCGGCACCT |
| | | ACTACGCCTCTTCCCTGATGGGCCGGTTCACCATCTCCCGGGAC |
| | | AACTCCAAGAACACCCTGTACCTGCAGATGAACTCCCTGCGGG |
| | | CCGAGGACACCGCCGTGTACTACTGCGCCAGACACGCCTACGG |
| | | CCACGACGGCGGCTTCGCCATGGATTATTGGGGCCAGGGCACC |
| | | CTGGTGACAGTGTCCTCCGCTAGCACCAAGGGCCCAAGTGTGT |
| | | TTCCCCTGGCCCCCAGCAGCAAGTCTACTTCCGGCGGAACTGC |
| | | TGCCCTGGGTTGCCTGGTGAAGGACTACTTCCCCGAGCCCGTG |
| | | ACAGTGTCCTGGAACTCTGGGGCTCTGACTTCCGGCGTGCACA |
| | | CCTTCCCCGCCGTGCTGCAGAGCAGCGGCCTGTACAGCCTGAG |
| | | CAGCGTGGTGACAGTGCCCTCCAGCTCTCTGGGAACCCAGACC |
| | | TATATCTGCAACGTGAACCACAAGCCCAGCAACACCAAGGTGG |
| | | ACAAGAGAGTGGAGCCCAAGAGCTGCGACAAGACCCACACCT |
| | | GCCCCCCCTGCCCAGCTCCAGAACTGCTGGGAGGGCCTTCCGT |
| | | GTTCCTGTTCCCCCCCAAGCCCAAGGACACCCTGATGATCAGC |
| | | AGGACCCCCGAGGTGACCTGCGTGGTGGTGGACGTGTCCCACG |
| | | AGGACCCAGAGGTGAAGTTCAACTGGTACGTGGACGGCGTGG |
| | | AGGTGCACAACGCCAAGACCAAGCCCAGAGAGGAGCAGTACA |
| | | ACAGCACCTACAGGGTGGTGTCCGTGCTGACCGTGCTGCACCA |
| | | GGACTGGCTGAACGGCAAAGAATACAAGTGCAAAGTCTCCAA |
| | | CAAGGCCCTGCCAGCCCCAATCGAAAAGACAATCAGCAAGGC |
| | | CAAGGGCCAGCCACGGGAGCCCCAGGTGTACACCCTGCCCCCC |
| | | AGCCGGGAGGAGATGACCAAGAACCAGGTGTCCCTGACCTGT |
| | | CTGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGCT |
| | | GACCGTGGACAAGTCCAGGTGGCAGCAGGGCAACGTGTTCAG |
| | | CTGCAGCGTGATGCACGAGGCCCTGCACAACCACTACACCCAG |
| | | AAGTCCCTGAGCCTGAGCCCCGGCAAG |
| SEQ ID NO: 8 | DNA | GAGATCGTGATGACCCAGTCCCCCGCCACCCTGTCTGTGTCTCC |
| | Light | CGGCGAGAGAGCCACCCTGAGCTGCAGAGCCTCCGAGTCCGTG |
| | Chain | TCCTCCAACGTGGCCTGGTATCAGCAGAGACCTGGTCAGGCCC |
| | | CTCGGCTGCTGATCTACGGCGCCTCTAACCGGGCCACCGGCAT |
| | | CCCTGCCAGATTCTCCGGCTCCGGCAGCGGCACCGACTTCACC |
| | | CTGACCATCTCCCGGCTGGAACCCGAGGACTTCGCCGTGTACT |
| | | ACTGCGGCCAGTCCTACTCATACCCCTTCACCTTCGGCCAGGG |
| | | CACCAAGCTGGAAATCAAGCGTACGGTGGCCGCTCCCAGCGTG |
| | | TTCATCTTCCCCCCCAGCGACGAGCAGCTGAAGAGCGGCACCG |
| | | CCAGCGTGGTGTGCCTGCTGAACAACTTCTACCCCCGGGAGGC |
| | | CAAGGTGCAGTGGAAGGTGGACAACGCCCTGCAGAGCGGCAA |
| | | CAGCCAGGAGAGCGTCACCGAGCAGGACAGCAAGGACTCCAC |
| | | CTACAGCCTGAGCAGCACCCTGACCCTGAGCAAGGCCGACTAC |
| | | GAGAAGCATAAGGTGTACGCCTGCGAGGTGACCCACCAGGGC |
| | | CTGTCCAGCCCCGTGACCAAGAGCTTCAACAGGGGCGAGTGC |
| SEQ ID NO: | HCDR1 | SYGVD |
| 9 (KABAT) | | |
| SEQ ID NO: | HCDR1 | GFSLSSY |
| 10 (CHOTHIA) | | |
| SEQ ID NO: 11 | HCDR2 | VIWGGGGTYYASSLMG |
| (KABAT) | | |
| SEQ ID NO: 12 | HCDR2 | WGGGG |
| (CHOTHIA) | | |
| SEQ ID NO: | HCDR3 | HAYGHDGGFAMDY |
| 3015 (KABAT) | | |
| SEQ ID NO: | HCDR3 | HAYGHDGGFAMDY |
| 3015 (CHOTHIA) | | |
| SEQ ID NO: | LCDR1 | RASESVSSNVA |
| 3016 (KABAT) | | |
| SEQ ID NO: | LCDR1 | SESVSSN |
| 3017 (CHOTHIA) | | |
| SEQ ID NO: 16 | LCDR2 | GASNRAT |
| (KABAT) | | |
| SEQ ID NO: 17 | LCDR2 | GAS |
| (CHOTHIA) | | |
| SEQ ID NO: | LCDR3 | GQSYSYPFT |
| 18 (KABAT) | | |
| SEQ ID NO: 19 | LCDR3 | SYSYPF |
| (CHOTHIA) | | |

Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 13.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signalling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 13
Amino acid sequence of other exemplary anti-GITR antibody molecules

| BMS-986156 | | |
|---|---|---|
| SEQ ID NO: 20 | VH | QVQLVESGGGVVQPGRSLRLSCAASGFTFSSYGMHWVRQAPGKG |
| | | LEWVAVIWYEGSNKYYADSVKGRFTISRDNSKNTLYLQMNSLRAE |
| | | DTAVYYCARGGSMVRGDYYYGMDVWGQGTTVTVSS |
| SEQ ID NO: 21 | VL | AIQLTQSPSSLSASVGDRVTITCRASQGISSALAWYQQKPGKAPKLL |
| | | IYDASSLESGVPSRFSGSGSGTDFTLTISSLQPEDFATYYCQQFNSYP |
| | | YTFGQGTKLEIK |

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFRbeta. In one embodiment, the inhibitor of an immune checkpoint molecule inhibits PD-1, PD-L1, LAG-3, TIM-3 or CTLA4, or any combination thereof. The term “inhibition” or “inhibitor” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40%, 50% or more is included by this term. Thus, inhibition need not be 100%.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) selected from, e.g., the heavy chain constant regions of IgG1, IgG2, IgG3, IgG4, IgM, IgA1, IgA2, IgD, and IgE; particularly, selected from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specificity, e.g., a second binding specificity to TIM-3, LAG-3, or PD-L2. In one embodiment, the bispecific antibody molecule binds to PD-1 or PD-L1 and TIM-3. In another embodiment, the bispecific antibody molecule binds to PD-1 or PD-L1 and LAG-3. In another embodiment, the bispecific antibody molecule binds to PD-1 and PD-L1. In yet another embodiment, the bispecific antibody molecule binds to PD-1 and PD-L2. In another embodiment, the bispecific antibody molecule binds to TIM-3 and LAG-3. Any combination of the aforesaid molecules can be made in a multispecific antibody molecule, e.g., a trispecific antibody that includes a first binding specificity to PD-1 or PD-1, and a second and third binding specificities to two or more of TIM-3, LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3 or CTLA4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule.

Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present disclosure. Any of the antibody molecules known in the art or disclosed herein can be used in the aforesaid combinations of inhibitors of checkpoint molecule.

### CTLA-4 Inhibitors

In some embodiments, the ZBTB32 inhibitor, or a pharmaceutically acceptable salt, hydrate, solvate, prodrug, stereoisomer, or tautomer thereof, of the present disclosure are used in combination with a CTLA-4 inhibitor to treat a disease, e.g., cancer. In some embodiments, the PD-1 inhibitor is selected from Ipilimumab (MDX-010, MDX-101, or Yervoy, Bristol-Myers Squibb), tremelilumab (ticilimumab. Pfizer/AstraZeneca), AGEN1181 (Agenus), Zalifrelimab (AGEN1884, Agenus), IBI310 (Innovent Biologics).

### PD-1 Inhibitors

In some embodiments, the ZBTB32 inhibitor, or a pharmaceutically acceptable salt, hydrate, solvate, prodrug, stereoisomer, or tautomer thereof, of the present disclosure are used in combination with a PD-1 inhibitor to treat a disease, e.g., cancer. In some embodiments, the PD-1 inhibitor is selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), or AMP-224 (Amplimmune).

Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 14 (e.g., from the heavy and light chain variable region sequences of BAP049-Clone-E or BAP049-Clone-B disclosed in Table 14), or encoded by a nucleotide sequence shown in Table 14. In some embodiments, the CDRs are according to the Kabat definition (e.g., as set out in Table 14). In some embodiments, the CDRs are according to the Chothia definition (e.g., as set out in Table 14). In some embodiments, the CDRs are according to the combined CDR definitions of both Kabat and Chothia (e.g., as set out in Table 14). In one embodiment, the combination of Kabat and Chothia CDR of VH CDR1 comprises the amino acid sequence GYTFTTYWMH (SEQ ID NO: 3198). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 14, or encoded by a nucleotide sequence shown in Table 14.

In one embodiment, the anti-PD-1 antibody molecule comprises a heavy chain variable region (VH) comprising a VHCDR1 amino acid sequence of SEQ ID NO: 22, a VHCDR2 amino acid sequence of SEQ ID NO: 3018, and a VHCDR3 amino acid sequence of SEQ ID NO: 3019; and a light chain variable region (VL) comprising a VLCDR1 amino acid sequence of SEQ ID NO: 31, a VLCDR2 amino acid sequence of SEQ ID NO: 32, and a VLCDR3 amino acid sequence of SEQ ID NO: 286, each disclosed in Table 14.

In one embodiment, the antibody molecule comprises a VH comprising a VHCDR1 encoded by the nucleotide sequence of SEQ ID NO: 3031, a VHCDR2 encoded by the nucleotide sequence of SEQ ID NO: 3032, and a VHCDR3 encoded by the nucleotide sequence of SEQ ID NO: 3033; and a VL comprising a VLCDR1 encoded by the nucleotide sequence of SEQ ID NO: 3036, a VLCDR2 encoded by the nucleotide sequence of SEQ ID NO: 3037, and a VLCDR3 encoded by the nucleotide sequence of SEQ ID NO: 3038, each disclosed in Table 14.

In one embodiment, the anti-PD-1 antibody molecule comprises a VH comprising the amino acid sequence of SEQ ID NO: 27, or an amino acid sequence at least 85%, 90%, 95%, or 99% identical or higher to SEQ ID NO: 27. In one embodiment, the anti-PD-1 antibody molecule comprises a VL comprising the amino acid sequence of SEQ ID NO: 3024, or an amino acid sequence at least 85%, 90%, 95%, or 99% identical or higherto SEQ ID NO: 3024. In one embodiment, the anti-PD-1 antibody molecule comprises a VL comprising the amino acid sequence of SEQ ID NO: 37, or an amino acid sequence at least 85%, 90%, 95%, or 99% identical or higher to SEQ ID NO: 37. In one embodiment, the anti-PD-1 antibody molecule comprises a VH comprising the amino acid sequence of SEQ ID NO: 27 and a VL comprising the amino acid sequence of SEQ ID NO: 3024. In one embodiment, the anti-PD-1 antibody molecule comprises a VH comprising the amino acid sequence of SEQ ID NO: 27 and a VL comprising the amino acid sequence of SEQ ID NO: 37.

In one embodiment, the antibody molecule comprises a VH encoded by the nucleotide sequence of SEQ ID NO: 28, or a nucleotide sequence at least 85%, 90%, 95%, or 99% identical or higher to SEQ ID NO: 28. In one embodiment, the antibody molecule comprises a VL encoded by the nucleotide sequence of SEQ ID NO: 3027 or 38, or a nucleotide sequence at least 85%, 90%, 95%, or 99% identical or higher to SEQ ID NO: 3027 or 38. In one embodiment, the antibody molecule comprises a VH encoded by the nucleotide sequence of SEQ ID NO: 28 and a VL encoded by the nucleotide sequence of SEQ ID NO: 3027 or 38.

In one embodiment, the anti-PD-1 antibody molecule comprises a heavy chain comprising the amino acid sequence of SEQ ID NO: 29, or an amino acid sequence at least 85%, 90%, 95%, or 99% identical or higher to SEQ ID NO: 29. In one embodiment, the anti-PD-1 antibody molecule comprises a light chain comprising the amino acid sequence of SEQ ID NO: 3029, or an amino acid sequence at least 85%, 90%, 95%, or 99% identical or higher to SEQ ID NO: 3029. In one embodiment, the anti-PD-1 antibody molecule comprises a light chain comprising the amino acid sequence of SEQ ID NO: 39, or an amino acid sequence at least 85%, 90%, 95%, or 99% identical or higher to SEQ ID NO: 39. In one embodiment, the anti-PD-1 antibody molecule comprises a heavy chain comprising the amino acid sequence of SEQ ID NO: 29 and a light chain comprising the amino acid sequence of SEQ ID NO: 3029. In one embodiment, the anti-PD-1 antibody molecule comprises a heavy chain comprising the amino acid sequence of SEQ ID NO: 29 and a light chain comprising the amino acid sequence of SEQ ID NO: 39.

In one embodiment, the antibody molecule comprises a heavy chain encoded by the nucleotide sequence of SEQ ID NO: 30, or a nucleotide sequence at least 85%, 90%, 95%, or 99% identical or higher to SEQ ID NO: 30. In one embodiment, the antibody molecule comprises a light chain encoded by the nucleotide sequence of SEQ ID NO: 3030 or 3023, or a nucleotide sequence at least 85%, 90%, 95%, or 99% identical or higher to SEQ ID NO: 3030 or 3023. In one embodiment, the antibody molecule comprises a heavy chain encoded by the nucleotide sequence of SEQ ID NO: 30 and a light chain encoded by the nucleotide sequence of SEQ ID NO: 3030 or 3023.

The antibody molecules described herein can be made by vectors, host cells, and methods described in US 2015/0210769, incorporated by reference in its entirety.

TABLE 14
Amino acid and nucleotide sequences of exemplary
anti-PD-1 antibody molecules

| | | |
|---|---|---|
| BAP049-Clone-B HC | | |
| SEQ ID NO: 22 | HCDR1 | TYWMH |
| (Kabat) | | |
| SEQ ID NO: | HCDR2 | NIYPGTGGSNFDEKFKN |
| 3018(Kabat) | | |
| SEQ ID NO: 3019 | HCDR3 | WTTGTGAY |
| (Kabat) | | |
| SEQ ID NO: 3020 | HCDR1 | GYTFTTY |
| (Chothia) | | |
| SEQ ID NO: 3021 | HCDR2 | YPGTGG |
| (Chothia) | | |
| SEQ ID NO: 3019 | HCDR3 | WTTGTGAY |
| (Chothia) | | |
| SEQ ID NO: 27 | VH | EVQLVQSGAEVKKPGESLRISCKGSGYTFTTYWMHWVRQAT |
| | | GQGLEWMGNIYPGTGGSNFDEKFKNRVTITADKSTSTAYME |
| | | LSSLRSEDTAVYYCTRWTTGTGAYWGQGTTVTVSS |
| SEQ ID NO: 28 | DNA | GAGGTGCAGCTGGTGCAGTCAGGCGCCGAAGTGAAGAAG |
| | VH | CCCGGCGAGTCACTGAGAATTAGCTGTAAAGGTTCAGGCT |
| | | ACACCTTCACTACCTACTGGATGCACTGGGTCCGCCAGGC |
| | | TACCGGTCAAGGCCTCGAGTGGATGGGTAATATCTACCCC |
| | | GGCACCGGCGGCTCTAACTTCGACGAGAAGTTTAAGAATA |
| | | GAGTGACTATCACCGCCGATAAGTCTACTAGCACCGCCTA |
| | | TATGGAACTGTCTAGCCTGAGATCAGAGGACACCGCCGTC |
| | | TACTACTGCACTAGGTGGACTACCGGCACAGGCGCCTACT |
| | | GGGGTCAAGGCACTACCGTGACCGTGTCTAGC |
| SEQ ID NO: 29 | Heavy | EVQLVQSGAEVKKPGESLRISCKGSGYTFTTYWMHWVRQAT |
| | chain | GQGLEWMGNIYPGTGGSNFDEKFKNRVTITADKSTSTAYME |
| | | LSSLRSEDTAVYYCTRWTTGTGAYWGQGTTVTVSSASTKGP |
| | | SVFPLAPCSRSTSESTAALGCLVKDYFPEPVTVSWNSGALTSG |
| | | VHTFPAVLQSSGLYSLSSVVTVPSSSLGTKTYTCNVDHKPSNT |
| | | KVDKRVESKYGPPCPPCPAPEFLGGPSVFLFPPKPKDTLMISR |
| | | TPEVTCVVVDVSQEDPEVQFNWYVDGVEVHNAKTKPREEQF |
| | | NSTYRVVSVLTVLHQDWLNGKEYKCKVSNKGLPSSIEKTISK |
| | | AKGQPREPQVYTLPPSQEEMTKNQVSLTCLVKGFYPSDIAVE |
| | | WESNGQPENNYKTTPPVLDSDGSFFLYSRLTVDKSRWQEGN |
| | | VFSCSVMHEALHNHYTQKSLSLSLG |
| SEQ ID NO: 30 | DNA | GAGGTGCAGCTGGTGCAGTCAGGCGCCGAAGTGAAGAAG |
| | heavy | CCCGGCGAGTCACTGAGAATTAGCTGTAAAGGTTCAGGCT |
| | chain | ACACCTTCACTACCTACTGGATGCACTGGGTCCGCCAGGC |
| | | TACCGGTCAAGGCCTCGAGTGGATGGGTAATATCTACCCC |
| | | GGCACCGGCGGCTCTAACTTCGACGAGAAGTTTAAGAATA |
| | | GAGTGACTATCACCGCCGATAAGTCTACTAGCACCGCCTA |
| | | TATGGAACTGTCTAGCCTGAGATCAGAGGACACCGCCGTC |
| | | TACTACTGCACTAGGTGGACTACCGGCACAGGCGCCTACT |
| | | GGGGTCAAGGCACTACCGTGACCGTGTCTAGCGCTAGCAC |
| | | TAAGGGCCCGTCCGTGTTCCCCCTGGCACCTTGTAGCCGG |
| | | AGCACTAGCGAATCCACCGCTGCCCTCGGCTGCCTGGTCA |
| | | AGGATTACTTCCCGGAGCCCGTGACCGTGTCCTGGAACAG |
| | | CGGAGCCCTGACCTCCGGAGTGCACACCTTCCCCGCTGTG |
| | | CTGCAGAGCTCCGGGCTGTACTCGCTGTCGTCGGTGGTCA |
| | | CGGTGCCTTCATCTAGCCTGGGTACCAAGACCTACACTTGC |
| | | AACGTGGACCACAAGCCTTCCAACACTAAGGTGGACAAGC |
| | | GCGTCGAATCGAAGTACGGCCCACCGTGCCCGCCTTGTCC |
| | | CGCGCCGGAGTTCCTCGGCGGTCCCTCGGTCTTTCTGTTCC |
| | | CACCGAAGCCCAAGGACACTTTGATGATTTCCCGCACCCC |
| | | TGAAGTGACATGCGTGGTCGTGGACGTGTCACAGGAAGAT |
| | | CCGGAGGTGCAGTTCAATTGGTACGTGGATGGCGTCGAGG |
| | | TGCACAACGCCAAAACCAAGCCGAGGGAGGAGCAGTTCA |
| | | ACTCCACTTACCGCGTCGTGTCCGTGCTGACGGTGCTGCAT |
| | | CAGGACTGGCTGAACGGGAAGGAGTACAAGTGCAAAGTG |
| | | TCCAACAAGGGACTTCCTAGCTCAATCGAAAAGACCATCT |
| | | CGAAAGCCAAGGGACAGCCCCGGGAACCCCAAGTGTATA |
| | | CCCTGCCACCGAGCCAGGAAGAAATGACTAAGAACCAAG |
| | | TCTCATTGACTTGCCTTGTGAAGGGCTTCTACCCATCGGAT |
| | | ATCGCCGTGGAATGGGAGTCCAACGGCCAGCCGGAAAAC |
| | | AACTACAAGACCACCCCTCCGGTGCTGGACTCAGACGGAT |
| | | CCTTCTTCCTCTACTCGCGGCTGACCGTGGATAAGAGCAG |
| | | ATGGCAGGAGGGAAATGTGTTCAGCTGTTCTGTGATGCAT |
| | | GAAGCCCTGCACAACCACTACACTCAGAAGTCCCTGTCCC |
| | | TCTCCCTGGGA |
| BAP049-Clone-B LC | | |
| SEQ ID NO: 31 | LCDR1 | KSSQSLLDSGNQKNFLT |
| (Kabat) | | |
| SEQ ID NO: 32 | LCDR2 | WASTRES |
| (Kabat) | | |
| SEQ ID NO: | LCDR3 | QNDYSYPYT |
| 286(Kabat) | | |
| SEQ ID NO: 34 | LCDR1 | SQSLLDSGNQKNF |
| (Chothia) | | |
| SEQ ID NO: 35 | LCDR2 | WAS |
| (Chothia) | | |
| SEQ ID NO: 36 | LCDR3 | DYSYPY |
| (Chothia) | | |
| SEQ ID NO: 37 | VL | EIVLTQSPATLSLSPGERATLSCKSSQSLLDSGNQKNFLTWYQ |
| | | QKPGKAPKLLIYWASTRESGVPSRFSGSGSGTDFTFTISSLQPE |
| | | DIATYYCQNDYSYPYTFGQGTKVEIK |
| SEQ ID NO: 38 | DNA | GAGATCGTCCTGACTCAGTCACCCGCTACCCTGAGCCTGA |
| | VL | GCCCTGGCGAGCGGGCTACACTGAGCTGTAAATCTAGTCA |
| | | GTCACTGCTGGATAGCGGTAATCAGAAGAACTTCCTGACC |
| | | TGGTATCAGCAGAAGCCCGGTAAAGCCCCTAAGCTGCTGA |
| | | TCTACTGGGCCTCTACTAGAGAATCAGGCGTGCCCTCTAG |
| | | GTTTAGCGGTAGCGGTAGTGGCACCGACTTCACCTTCACT |
| | | ATCTCTAGCCTGCAGCCCGAGGATATCGCTACCTACTACTG |
| | | TCAGAACGACTATAGCTACCCCTACACCTTCGGTCAAGGC |
| | | ACTAAGGTCGAGATTAAG |
| SEQ ID NO: 3228 | Light | EIVLTQSPATLSLSPGERATLSCKSSQSLLDSGNQKNFLTWYQ |
| | chain | QKPGKAPKLLIYWASTRESGVPSRFSGSGSGTDFTFTISSLQPE |
| | | DIATYYCQNDYSYPYTFGQGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: 3023 | DNA | GAGATCGTCCTGACTCAGTCACCCGCTACCCTGAGCCTGA |
| | light | GCCCTGGCGAGCGGGCTACACTGAGCTGTAAATCTAGTCA |
| | chain | GTCACTGCTGGATAGCGGTAATCAGAAGAACTTCCTGACC |
| | | TGGTATCAGCAGAAGCCCGGTAAAGCCCCTAAGCTGCTGA |
| | | TCTACTGGGCCTCTACTAGAGAATCAGGCGTGCCCTCTAG |
| | | GTTTAGCGGTAGCGGTAGTGGCACCGACTTCACCTTCACT |
| | | ATCTCTAGCCTGCAGCCCGAGGATATCGCTACCTACTACTG |
| | | TCAGAACGACTATAGCTACCCCTACACCTTCGGTCAAGGC |
| | | ACTAAGGTCGAGATTAAGCGTACGGTGGCCGCTCCCAGCG |
| | | TGTTCATCTTCCCCCCCAGCGACGAGCAGCTGAAGAGCGG |
| | | CACCGCCAGCGTGGTGTGCCTGCTGAACAACTTCTACCCC |
| | | CGGGAGGCCAAGGTGCAGTGGAAGGTGGACAACGCCCTG |
| | | CAGAGCGGCAACAGCCAGGAGAGCGTCACCGAGCAGGAC |
| | | AGCAAGGACTCCACCTACAGCCTGAGCAGCACCCTGACCC |
| | | TGAGCAAGGCCGACTACGAGAAGCATAAGGTGTACGCCTG |
| | | CGAGGTGACCCACCAGGGCCTGTCCAGCCCCGTGACCAAG |
| | | AGCTTCAACAGGGGCGAGTGC |
| BAP049-Clone-E HC | | |
| SEQ ID NO: 22 | HCDR1 | TYWMH |
| (Kabat) | | |
| SEQ ID NO: 3018 | HCDR2 | NIYPGTGGSNFDEKFKN |
| (Kabat) | | |
| SEQ ID NO: 3019 | HCDR3 | WTTGTGAY |
| (Kabat) | | |
| SEQ ID NO: 3020 | HCDR1 | GYTFTTY |
| (Chothia) | | |
| SEQ ID NO: 3021 | HCDR2 | YPGTGG |
| (Chothia) | | |
| SEQ ID NO: 3019 | HCDR3 | WTTGTGAY |
| (Chothia) | | |
| SEQ ID NO: 27 | VH | EVQLVQSGAEVKKPGESLRISCKGSGYTFTTYWMHWVRQAT |
| | | GQGLEWMGNIYPGTGGSNFDEKFKNRVTITADKSTSTAYME |
| | | LSSLRSEDTAVYYCTRWTTGTGAYWGQGTTVTVSS |
| SEQ ID NO: 28 | DNA | GAGGTGCAGCTGGTGCAGTCAGGCGCCGAAGTGAAGAAG |
| | VH | CCCGGCGAGTCACTGAGAATTAGCTGTAAAGGTTCAGGCT |
| | | ACACCTTCACTACCTACTGGATGCACTGGGTCCGCCAGGC |
| | | TACCGGTCAAGGCCTCGAGTGGATGGGTAATATCTACCCC |
| | | GGCACCGGCGGCTCTAACTTCGACGAGAAGTTTAAGAATA |
| | | GAGTGACTATCACCGCCGATAAGTCTACTAGCACCGCCTA |
| | | TATGGAACTGTCTAGCCTGAGATCAGAGGACACCGCCGTC |
| | | TACTACTGCACTAGGTGGACTACCGGCACAGGCGCCTACT |
| | | GGGGTCAAGGCACTACCGTGACCGTGTCTAGC |
| SEQ ID NO: 29 | Heavy | EVQLVQSGAEVKKPGESLRISCKGSGYTFTTYWMHWVRQAT |
| | chain | GQGLEWMGNIYPGTGGSNFDEKFKNRVTITADKSTSTAYME |
| | | LSSLRSEDTAVYYCTRWTTGTGAYWGQGTTVTVSSASTKGP |
| | | SVFPLAPCSRSTSESTAALGCLVKDYFPEPVTVSWNSGALTSG |
| | | VHTFPAVLQSSGLYSLSSVVTVPSSSLGTKTYTCNVDHKPSNT |
| | | KVDKRVESKYGPPCPPCPAPEFLGGPSVFLFPPKPKDTLMISR |
| | | TPEVTCVVVDVSQEDPEVQFNWYVDGVEVHNAKTKPREEQF |
| | | NSTYRVVSVLTVLHQDWLNGKEYKCKVSNKGLPSSIEKTISK |
| | | AKGQPREPQVYTLPPSQEEMTKNQVSLTCLVKGFYPSDIAVE |
| | | WESNGQPENNYKTTPPVLDSDGSFFLYSRLTVDKSRWQEGN |
| | | VFSCSVMHEALHNHYTQKSLSLSLG |
| SEQ ID NO: 30 | DNA | GAGGTGCAGCTGGTGCAGTCAGGCGCCGAAGTGAAGAAG |
| | heavy | CCCGGCGAGTCACTGAGAATTAGCTGTAAAGGTTCAGGCT |
| | chain | ACACCTTCACTACCTACTGGATGCACTGGGTCCGCCAGGC |
| | | TACCGGTCAAGGCCTCGAGTGGATGGGTAATATCTACCCC |
| | | GGCACCGGCGGCTCTAACTTCGACGAGAAGTTTAAGAATA |
| | | GAGTGACTATCACCGCCGATAAGTCTACTAGCACCGCCTA |
| | | TATGGAACTGTCTAGCCTGAGATCAGAGGACACCGCCGTC |
| | | TACTACTGCACTAGGTGGACTACCGGCACAGGCGCCTACT |
| | | GGGGTCAAGGCACTACCGTGACCGTGTCTAGCGCTAGCAC |
| | | TAAGGGCCCGTCCGTGTTCCCCCTGGCACCTTGTAGCCGG |
| | | AGCACTAGCGAATCCACCGCTGCCCTCGGCTGCCTGGTCA |
| | | AGGATTACTTCCCGGAGCCCGTGACCGTGTCCTGGAACAG |
| | | CGGAGCCCTGACCTCCGGAGTGCACACCTTCCCCGCTGTG |
| | | CTGCAGAGCTCCGGGCTGTACTCGCTGTCGTCGGTGGTCA |
| | | CGGTGCCTTCATCTAGCCTGGGTACCAAGACCTACACTTGC |
| | | AACGTGGACCACAAGCCTTCCAACACTAAGGTGGACAAGC |
| | | GCGTCGAATCGAAGTACGGCCCACCGTGCCCGCCTTGTCC |
| | | CGCGCCGGAGTTCCTCGGCGGTCCCTCGGTCTTTCTGTTCC |
| | | CACCGAAGCCCAAGGACACTTTGATGATTTCCCGCACCCC |
| | | TGAAGTGACATGCGTGGTCGTGGACGTGTCACAGGAAGAT |
| | | CCGGAGGTGCAGTTCAATTGGTACGTGGATGGCGTCGAGG |
| | | TGCACAACGCCAAAACCAAGCCGAGGGAGGAGCAGTTCA |
| | | ACTCCACTTACCGCGTCGTGTCCGTGCTGACGGTGCTGCAT |
| | | CAGGACTGGCTGAACGGGAAGGAGTACAAGTGCAAAGTG |
| | | TCCAACAAGGGACTTCCTAGCTCAATCGAAAAGACCATCT |
| | | CGAAAGCCAAGGGACAGCCCCGGGAACCCCAAGTGTATA |
| | | CCCTGCCACCGAGCCAGGAAGAAATGACTAAGAACCAAG |
| | | TCTCATTGACTTGCCTTGTGAAGGGCTTCTACCCATCGGAT |
| | | ATCGCCGTGGAATGGGAGTCCAACGGCCAGCCGGAAAAC |
| | | AACTACAAGACCACCCCTCCGGTGCTGGACTCAGACGGAT |
| | | CCTTCTTCCTCTACTCGCGGCTGACCGTGGATAAGAGCAG |
| | | ATGGCAGGAGGGAAATGTGTTCAGCTGTTCTGTGATGCAT |
| | | GAAGCCCTGCACAACCACTACACTCAGAAGTCCCTGTCCC |
| | | TCTCCCTGGGA |
| BAP049-Clone-E LC | | |
| SEQ ID NO: 31 | LCDR1 | KSSQSLLDSGNQKNFLT |
| (Kabat) | | |
| SEQ ID NO: 32 | LCDR2 | WASTRES |
| (Kabat) | | |
| SEQ ID NO: | LCDR3 | QNDYSYPYT |
| 286(Kabat) | | |
| SEQ ID NO: 34 | LCDR1 | SQSLLDSGNQKNF |
| (Chothia) | | |
| SEQ ID NO: 35 | LCDR2 | WAS |
| (Chothia) | | |
| SEQ ID NO: 36 | LCDR3 | DYSYPY |
| (Chothia) | | |
| SEQ ID NO: 3025 | VL | EIVLTQSPATLSLSPGERATLSCKSSQSLLDSGNQKNFLTWYQ |
| | | QKPGQAPRLLIYWASTRESGVPSRFSGSGSGTDFTFTISSLEAE |
| | | DAATYYCQNDYSYPYTFGQGTKVEIK |
| SEQ ID NO: 3027 | DNA | GAGATCGTCCTGACTCAGTCACCCGCTACCCTGAGCCTGA |
| | VL | GCCCTGGCGAGCGGGCTACACTGAGCTGTAAATCTAGTCA |
| | | GTCACTGCTGGATAGCGGTAATCAGAAGAACTTCCTGACC |
| | | TGGTATCAGCAGAAGCCCGGTCAAGCCCCTAGACTGCTGA |
| | | TCTACTGGGCCTCTACTAGAGAATCAGGCGTGCCCTCTAG |
| | | GTTTAGCGGTAGCGGTAGTGGCACCGACTTCACCTTCACT |
| | | ATCTCTAGCCTGGAAGCCGAGGACGCCGCTACCTACTACT |
| | | GTCAGAACGACTATAGCTACCCCTACACCTTCGGTCAAGG |
| | | CACTAAGGTCGAGATTAAG |
| SEQ ID NO: 3029 | Light | EIVLTQSPATLSLSPGERATLSCKSSQSLLDSGNQKNFLTWYQ |
| | chain | QKPGQAPRLLIYWASTRESGVPSRFSGSGSGTDFTFTISSLEAE |
| | | DAATYYCQNDYSYPYTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 3030 | DNA | GAGATCGTCCTGACTCAGTCACCCGCTACCCTGAGCCTGA |
| | light | GCCCTGGCGAGCGGGCTACACTGAGCTGTAAATCTAGTCA |
| | chain | GTCACTGCTGGATAGCGGTAATCAGAAGAACTTCCTGACC |
| | | TGGTATCAGCAGAAGCCCGGTCAAGCCCCTAGACTGCTGA |
| | | TCTACTGGGCCTCTACTAGAGAATCAGGCGTGCCCTCTAG |
| | | GTTTAGCGGTAGCGGTAGTGGCACCGACTTCACCTTCACT |
| | | ATCTCTAGCCTGGAAGCCGAGGACGCCGCTACCTACTACT |
| | | GTCAGAACGACTATAGCTACCCCTACACCTTCGGTCAAGG |
| | | CACTAAGGTCGAGATTAAGCGTACGGTGGCCGCTCCCAGC |
| | | GTGTTCATCTTCCCCCCCAGCGACGAGCAGCTGAAGAGCG |
| | | GCACCGCCAGCGTGGTGTGCCTGCTGAACAACTTCTACCC |
| | | CCGGGAGGCCAAGGTGCAGTGGAAGGTGGACAACGCCCT |
| | | GCAGAGCGGCAACAGCCAGGAGAGCGTCACCGAGCAGGA |
| | | CAGCAAGGACTCCACCTACAGCCTGAGCAGCACCCTGACC |
| | | CTGAGCAAGGCCGACTACGAGAAGCATAAGGTGTACGCCT |
| | | GCGAGGTGACCCACCAGGGCCTGTCCAGCCCCGTGACCAA |
| | | GAGCTTCAACAGGGGCGAGTGC |
| BAP049-Clone-B HC | | |
| SEQ ID NO: | HCDR1 | ACCTACTGGATGCAC |
| 3031 (Kabat) | | |
| SEQ ID NO: 3032 | HCDR2 | AATATCTACCCCGGCACCGGCGGCTCTAACTTCGACGAGA |
| (Kabat) | | AGTTTAAGAAT |
| SEQ ID NO: 3033 | HCDR3 | TGGACTACCGGCACAGGCGCCTAC |
| (Kabat) | | |
| SEQ ID NO: | HCDR1 | GGCTACACCTTCACTACCTAC |
| 3034(Chothia) | | |
| SEQ ID NO: 3035 | HCDR2 | TACCCCGGCACCGGCGGC |
| (Chothia) | | |
| SEQ ID NO: 3033 | HCDR3 | TGGACTACCGGCACAGGCGCCTAC |
| (Chothia) | | |
| BAP049-Clone-B LC | | |
| SEQ ID NO: 3036 | LCDR1 | AAATCTAGTCAGTCACTGCTGGATAGCGGTAATCAGAAGA |
| (Kabat) | | ACTTCCTGACC |
| SEQ ID NO: 3037 | LCDR2 | TGGGCCTCTACTAGAGAATCA |
| (Kabat) | | |
| SEQ ID NO: 3038 | LCDR3 | CAGAACGACTATAGCTACCCCTACACC |
| (Kabat) | | |
| SEQ ID NO: 3039 | LCDR1 | AGTCAGTCACTGCTGGATAGCGGTAATCAGAAGAACTTC |
| (Chothia) | | |
| SEQ ID NO: 3040 | LCDR2 | TGGGCCTCT |
| (Chothia) | | |
| SEQ ID NO: | LCDR3 | GACTATAGCTACCCCTAC |
| 3041(Chothia) | | |
| BAP049-Clone-E HC | | |
| SEQ ID NO: 3031 | HCDR1 | ACCTACTGGATGCAC |
| (Kabat) | | |
| SEQ ID NO: 3032 | HCDR2 | AATATCTACCCCGGCACCGGCGGCTCTAACTTCGACGAGA |
| (Kabat) | | AGTTTAAGAAT |
| SEQ ID NO: | HCDR3 | TGGACTACCGGCACAGGCGCCTAC |
| 3033(Kabat) | | |
| SEQ ID NO: 3034 | HCDR1 | GGCTACACCTTCACTACCTAC |
| (Chothia) | | |
| SEQ ID NO: 3035 | HCDR2 | TACCCCGGCACCGGCGGC |
| (Chothia) | | |
| SEQ ID NO: | HCDR3 | TGGACTACCGGCACAGGCGCCTAC |
| 3033(Chothia) | | |
| BAP049-Clone-E LC | | |
| SEQ ID NO: 3036 | LCDR1 | AAATCTAGTCAGTCACTGCTGGATAGCGGTAATCAGAAGA |
| (Kabat) | | ACTTCCTGACC |
| SEQ ID NO: 3037 | LCDR2 | TGGGCCTCTACTAGAGAATCA |
| (Kabat) | | |
| SEQ ID NO: 3038 | LCDR3 | CAGAACGACTATAGCTACCCCTACACC |
| (Kabat) | | |
| SEQ ID NO: 3039 | LCDR1 | AGTCAGTCACTGCTGGATAGCGGTAATCAGAAGAACTTC |
| (Chothia) | | |
| SEQ ID NO: 3040 | LCDR2 | TGGGCCTCT |
| (Chothia) | | |
| SEQ ID NO: 3041 | LCDR3 | GACTATAGCTACCCCTAC |
| (Chothia) | | |

Other Exemplary PD-1 Inhibitors

In some embodiments, the anti-PD-1 antibody is Nivolumab (CAS Registry Number: 946414-94-4). Alternative names for Nivolumab include MDX-1106, MDX-1106-04, ONO-4538, BMS-936558 or OPDIVO®. Nivolumab is a fully human IgG4 monoclonal antibody, which specifically blocks PD1. Nivolumab (clone 5C4) and other human monoclonal antibodies that specifically bind to PD1 are disclosed in U.S. Pat. No. 8,008,449 and PCT Publication No. WO2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 15.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, PCT Publication No. WO2009/114335, and U.S. Pat. No. 8,354,509, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 15.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in PCT Publication No. WO2009/101611, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 15.

Other anti-PD1 antibodies are disclosed in U.S. Pat. No. 8,609,089, US Publication No. 2010028330, and/or US Publication No. 20120114649, incorporated by reference in their entirety. Other anti-PD1 antibodies include AMP 514 (Amplimmune).

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is Cemiplimab (Regeneron), also known as REGN2810. In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is Tislelizumab (Beigene), also known as BGB-A317 or BGB-108. In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHRO1210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is Dostarlimab (Tesaro), also known as TSR-042, also known as ANBO11. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

In one embodiment, the anti-PD-1 antibody molecule is Balstilimab (Agenus), also known as AGEN2035. In one embodiment, the anti-PD-1 antibody molecule comprises one or more of the CDR sequences (or collectively all of the CDR sequences), the heavy chain or light chain variable region sequence, or the heavy chain or light chain sequence of Balstilimab.

In one embodiment, the anti-PD-1 antibody molecule is Sintilimab (InnoVent), In one embodiment, the anti-PD-1 antibody molecule comprises one or more of the CDR sequences (or collectively all of the CDR sequences), the heavy chain or light chain variable region sequence, or the heavy chain or light chain sequence of Sintilimab.

In one embodiment, the anti-PD-1 antibody molecule is Toripalimab (Shanghai Junshi Bioscience). In one embodiment, the anti-PD-1 antibody molecule comprises one or more of the CDR sequences (or collectively all of the CDR sequences), the heavy chain or light chain variable region sequence, or the heavy chain or light chain sequence of Toripalimab.

In one embodiment, the anti-PD-1 antibody molecule is Camrelizumab (Jiangsu Hengrui Medicine Co.). In one embodiment, the anti-PD-1 antibody molecule comprises one or more of the CDR sequences (or collectively all of the CDR sequences), the heavy chain or light chain variable region sequence, or the heavy chain or light chain sequence of Camrelizumab.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signalling pathway, e.g., as described in U.S. Pat. No. 8,907,053, incorporated by reference in its entirety. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224 (B7-DCIg (Amplimmune), e.g., disclosed in WO 2010/027827 and WO 2011/066342, incorporated by reference in their entirety). Table 15. Amino acid sequences of other exemplary anti-PD-1 antibody molecules

TABLE 15
Amino acid sequences of other exemplary anti-PD-1 antibody molecules

| | | |
|---|---|---|
| Nivolumab | | |
| SEQ ID NO: | Heavy | QVQLVESGGGVVQPGRSLRLDCKASGITFSNSGMHWVRQAPGKGL |
| 3042 | chain | EWVAVIWYDGSKRYYADSVKGRFTISRDNSKNTLFLQMNSLRAED |
| | | TAVYYCATNDDYWGQGTLVTVSSASTKGPSVFPLAPCSRSTSESTA |
| | | ALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLG |
| | | GPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPS |
| | | DIAVEWESNGQPENNYKTTPPVLDSDGSFFLYSRLTVDKSRWQEGN |
| | | VFSCSVMHEALHNHYTQKSLSLSLGK |
| SEQ ID NO: | Light | EIVLTQSPATLSLSPGERATLSCRASQSVSSYLAWYQQKPGQAPRLLI |
| 3043 | chain | YDASNRATGIPARFSGSGSGTDFTLTISSLEPEDFAVYYCQQSSNWP |
| | | RTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC |
| Pembrolizumab | | |
| SEQ ID NO: | Heavy | QVQLVQSGVEVKKPGASVKVSCKASGYTFTNYYMYWVRQAPGQG |
| 3044 | chain | LEWMGGINPSNGGTNFNEKFKNRVTLTTDSSTTTAYMELKSLQFDD |
| | | TAVYYCARRDYRFDMGFDYWGQGTTVTVSSASTKGPSVFPLAPCS |
| | | RSTSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPPC |
| | | PAPEFLGGPSVFLFPPKPKDTLMISRTPEVTCVVVDVSQEDPEVQFN |
| | | WYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTC |
| | | LVKGFYPSDIAVEWESNGQPENNYKTTPPVLDSDGSFFLYSRLTVDK |
| | | SRWQEGNVFSCSVMHEALHNHYTQKSLSLSLGK |
| SEQ ID NO: | Light | EIVLTQSPATLSLSPGERATLSCRASKGVSTSGYSYLHWYQQKPGQA |
| 3045 | chain | PRLLIYLASYLESGVPARFSGSGSGTDFTLTISSLEPEDFAVYYCQHS |
| | | RDLPLTFGGGTKVEIKRTVAAPSVFIFPPSDEQLKSGTASVVCLLNNF |
| | | YPREAKVQWKVDNALQSGNSQESVTEQDSKDSTYSLSSTLTLSKAD |
| | | YEKHKVYACEVTHQGLSSPVTKSFNRGEC |
| Pidilizumab | | |
| SEQ ID NO: | Heavy | QVQLVQSGSELKKPGASVKISCKASGYTFTNYGMNWVRQAPGQGL |
| 3046 | chain | QWMGWINTDSGESTYAEEFKGRFVFSLDTSVNTAYLQITSLTAEDT |
| | | GMYFCVRVGYDALDYWGQGTLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCP |
| | | APELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| SEQ ID NO: | Light | EIVLTQSPSSLSASVGDRVTITCSARSSVSYMHWFQQKPGKAPKLWI |
| 3047 | chain | YRTSNLASGVPSRFSGSGSGTSYCLTINSLQPEDFATYYCQQRSSFPL |
| | | TFGGGTKLEIKRTVAAPSVFIFPPSDEQLKSGTASVVCLLNNFYPREA |
| | | KVQWKVDNALQSGNSQESVTEQDSKDSTYSLSSTLTLSKADYEKH |
| | | KVYACEVTHQGLSSPVTKSFNRGEC |

### PD-L1 Inhibitors

In some embodiments, the ZBTB32 inhibitor, or a pharmaceutically acceptable salt, hydrate, solvate, prodrug, stereoisomer, or tautomer thereof, of the present disclosure are used in combination with a PD-L1 inhibitor for treating a disease, e.g., cancer. In some embodiments, the PD-L1 inhibitor is selected from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 16 (e.g., from the heavy and light chain variable region sequences of BAP058-Clone O or BAP058-Clone N disclosed in Table 16), or encoded by a nucleotide sequence shown in Table 16. In some embodiments, the CDRs are according to the Kabat definition (e.g., as set out in Table 16). In some embodiments, the CDRs are according to the Chothia definition (e.g., as set out in Table 16). In some embodiments, the CDRs are according to the combined CDR definitions of both Kabat and Chothia (e.g., as set out in Table 16). In one embodiment, the combination of Kabat and Chothia CDR of VH CDR1 comprises the amino acid sequence GYTFTSYWMY (SEQ ID NO: 3199). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 16, or encoded by a nucleotide sequence shown in Table 16.

In one embodiment, the anti-PD-L1 antibody molecule comprises a heavy chain variable region (VH) comprising a VHCDR1 amino acid sequence of SEQ ID NO: 3048, a VHCDR2 amino acid sequence of SEQ ID NO: 3049, and a VHCDR3 amino acid sequence of SEQ ID NO: 3050; and a light chain variable region (VL) comprising a VLCDR1 amino acid sequence of SEQ ID NO: 3056, a VLCDR2 amino acid sequence of SEQ ID NO: 3057, and a VLCDR3 amino acid sequence of SEQ ID NO: 3058, each disclosed in Table 16.

In one embodiment, the anti-PD-L1 antibody molecule comprises a VH comprising a VHCDR1 encoded by the nucleotide sequence of SEQ ID NO: 3075, a VHCDR2 encoded by the nucleotide sequence of SEQ ID NO: 3076, and a VHCDR3 encoded by the nucleotide sequence of SEQ ID NO: 3077; and a VL comprising a VLCDR1 encoded by the nucleotide sequence of SEQ ID NO: 3080, a VLCDR2 encoded by the nucleotide sequence of SEQ ID NO: 3081, and a VLCDR3 encoded by the nucleotide sequence of SEQ ID NO: 3082, each disclosed in Table 16.

In one embodiment, the anti-PD-L1 antibody molecule comprises a VH comprising the amino acid sequence of SEQ ID NO: 3053, or an amino acid sequence at least 85%, 90%, 95%, or 99% identical or higher to SEQ ID NO: 3053. In one embodiment, the anti-PD-L1 antibody molecule comprises a VL comprising the amino acid sequence of SEQ ID NO: 3063, or an amino acid sequence at least 85%, 90%, 95%, or 99% identical or higher to SEQ ID NO: 3063. In one embodiment, the anti-PD-L1 antibody molecule comprises a VH comprising the amino acid sequence of SEQ ID NO: 3067, or an amino acid sequence at least 85%, 90%, 95%, or 99% identical or higher to SEQ ID NO: 3067. In one embodiment, the anti-PD-L1 antibody molecule comprises a VL comprising the amino acid sequence of SEQ ID NO: 3071, or an amino acid sequence at least 85%, 90%, 95%, or 99% identical or higher to SEQ ID NO: 3071. In one embodiment, the anti-PD-L1 antibody molecule comprises a VH comprising the amino acid sequence of SEQ ID NO: 3053 and a VL comprising the amino acid sequence of SEQ ID NO: 3063. In one embodiment, the anti-PD-L1 antibody molecule comprises a VH comprising the amino acid sequence of SEQ ID NO: 3067 and a VL comprising the amino acid sequence of SEQ ID NO: 3071.

In one embodiment, the antibody molecule comprises a VH encoded by the nucleotide sequence of SEQ ID NO: 3054, or a nucleotide sequence at least 85%, 90%, 95%, or 99% identical or higher to SEQ ID NO: 3054. In one embodiment, the antibody molecule comprises a VL encoded by the nucleotide sequence of SEQ ID NO: 3064, or a nucleotide sequence at least 85%, 90%, 95%, or 99% identical or higher to SEQ ID NO: 3064. In one embodiment, the antibody molecule comprises a VH encoded by the nucleotide sequence of SEQ ID NO: 3068, or a nucleotide sequence at least 85%, 90%, 95%, or 99% identical or higher to SEQ ID NO: 3068. In one embodiment, the antibody molecule comprises a VL encoded by the nucleotide sequence of SEQ ID NO: 3072, or a nucleotide sequence at least 85%, 90%, 95%, or 99% identical or higher to SEQ ID NO: 3072. In one embodiment, the antibody molecule comprises a VH encoded by the nucleotide sequence of SEQ ID NO: 3054 and a VL encoded by the nucleotide sequence of SEQ ID NO: 3064. In one embodiment, the antibody molecule comprises a VH encoded by the nucleotide sequence of SEQ ID NO: 3068 and a VL encoded by the nucleotide sequence of SEQ ID NO: 3072.

In one embodiment, the anti-PD-L1 antibody molecule comprises a heavy chain comprising the amino acid sequence of SEQ ID NO: 3055, or an amino acid sequence at least 85%, 90%, 95%, or 99% identical or higher to SEQ ID NO: 3055. In one embodiment, the anti-PD-L1 antibody molecule comprises a light chain comprising the amino acid sequence of SEQ ID NO: 3065, or an amino acid sequence at least 85%, 90%, 95%, or 99% identical or higher to SEQ ID NO: 3065. In one embodiment, the anti-PD-L1 antibody molecule comprises a heavy chain comprising the amino acid sequence of SEQ ID NO: 3069, or an amino acid sequence at least 85%, 90%, 95%, or 99% identical or higher to SEQ ID NO: 3069. In one embodiment, the anti-PD-L1 antibody molecule comprises a light chain comprising the amino acid sequence of SEQ ID NO: 3073, or an amino acid sequence at least 85%, 90%, 95%, or 99% identical or higher to SEQ ID NO: 3073. In one embodiment, the anti-PD-L1 antibody molecule comprises a heavy chain comprising the amino acid sequence of SEQ ID NO: 3055 and a light chain comprising the amino acid sequence of SEQ ID NO: 3065. In one embodiment, the anti-PD-L1 antibody molecule comprises a heavy chain comprising the amino acid sequence of SEQ ID NO: 3069 and a light chain comprising the amino acid sequence of SEQ ID NO: 3073.

In one embodiment, the antibody molecule comprises a heavy chain encoded by the nucleotide sequence of SEQ ID NO: 3062, or a nucleotide sequence at least 85, 90%, 95% or 99% identical or higher to SEQ ID NO: 3062. In one embodiment, the antibody molecule comprises a light chain encoded by the nucleotide sequence of SEQ ID NO: 3066, or a nucleotide sequence at least 85%, 90%, 95%, or 99% identical or higher to SEQ ID NO: 3066. In one embodiment, the antibody molecule comprises a heavy chain encoded by the nucleotide sequence of SEQ ID NO: 3070, or a nucleotide sequence at least 85%, 90%, 95%, or 99% identical or higher to SEQ ID NO: 3070. In one embodiment, the antibody molecule comprises a light chain encoded by the nucleotide sequence of SEQ ID NO: 3074, or a nucleotide sequence at least 85D, 90N, 95S or 99 identical or higher to SEQ ID NO: 3074. In one embodiment, the antibody molecule comprises a heavy chain encoded by the nucleotide sequence of SEQ ID NO: 3062 and a light chain encoded by the nucleotide sequence of SEQ ID NO: 3066. In one embodiment, the antibody molecule comprises a heavy chain encoded by the nucleotide sequence of SEQ ID NO: 3070 and a light chain encoded by the nucleotide sequence of SEQ ID NO: 3074.

The antibody molecules described herein can be made by vectors, host cells, and methods described in US 2016/0108123, incorporated by reference in its entirety.

TABLE 16
Amino acid and nucleotide sequences of exemplary
anti-PD-L1 antibody molecules

| | | |
|---|---|---|
| BAP058-Clone O HC | | |
| SEQ ID NO: 3048 | HCDR1 | SYWMY |
| (Kabat) | | |
| SEQ ID NO: | HCDR2 | RIDPNSGSTKYNEKFKN |
| 3049(Kabat) | | |
| SEQ ID NO: 3050 | HCDR3 | DYRKGLYAMDY |
| (Kabat) | | |
| SEQ ID NO: 3051 | HCDR1 | GYTFTSY |
| (Chothia) | | |
| SEQ ID NO: 3052 | HCDR2 | DPNSGS |
| (Chothia) | | |
| SEQ ID NO: 3050 | HCDR3 | DYRKGLYAMDY |
| (Chothia) | | |
| SEQ ID NO: 3053 | VH | EVQLVQSGAEVKKPGATVKISCKVSGYTFTSYWMYWVR |
| | | QARGQRLEWIGRIDPNSGSTKYNEKFKNRFTISRDNSKNTL |
| | | YLQMNSLRAEDTAVYYCARDYRKGLYAMDYWGQGTTVTVSS |
| SEQ ID NO: 3054 | DNA | GAAGTGCAGCTGGTGCAGTCAGGCGCCGAAGTGAAGAA |
| | VH | ACCCGGCGCTACCGTGAAGATTAGCTGTAAAGTCTCAG |
| | | GCTACACCTTCACTAGCTACTGGATGTACTGGGTCCGAC |
| | | AGGCTAGAGGGCAAAGACTGGAGTGGATCGGTAGAATC |
| | | GACCCTAATAGCGGCTCTACTAAGTATAACGAGAAGTT |
| | | TAAGAATAGGTTCACTATTAGTAGGGATAACTCTAAGA |
| | | ACACCCTGTACCTGCAGATGAATAGCCTGAGAGCCGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCA |
| SEQ ID NO: 3055 | Heavy | EVQLVQSGAEVKKPGATVKISCKVSGYTFTSYWMYWVR |
| | chain | QARGQRLEWIGRIDPNSGSTKYNEKFKNRFTISRDNSKNTL |
| | | YLQMNSLRAEDTAVYYCARDYRKGLYAMDYWGQGTTV |
| | | TVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFFLYSRLTVDKSRWQEGNVFSCSVMHEALHNHY |
| | | TQKSLSLSLG |
| SEQ ID NO: 3062 | DNA | GAAGTGCAGCTGGTGCAGTCAGGCGCCGAAGTGAAGAA |
| | heavy | ACCCGGCGCTACCGTGAAGATTAGCTGTAAAGTCTCAG |
| | chain | GCTACACCTTCACTAGCTACTGGATGTACTGGGTCCGAC |
| | | AGGCTAGAGGGCAAAGACTGGAGTGGATCGGTAGAATC |
| | | GACCCTAATAGCGGCTCTACTAAGTATAACGAGAAGTT |
| | | TAAGAATAGGTTCACTATTAGTAGGGATAACTCTAAGA |
| | | ACACCCTGTACCTGCAGATGAATAGCCTGAGAGCCGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCAGCTAGCACTAAGGGCCCGTCCG |
| | | TGTTCCCCCTGGCACCTTGTAGCCGGAGCACTAGCGAAT |
| | | CCACCGCTGCCCTCGGCTGCCTGGTCAAGGATTACTTCC |
| | | CGGAGCCCGTGACCGTGTCCTGGAACAGCGGAGCCCTG |
| | | ACCTCCGGAGTGCACACCTTCCCCGCTGTGCTGCAGAGC |
| | | TCCGGGCTGTACTCGCTGTCGTCGGTGGTCACGGTGCCT |
| | | TCATCTAGCCTGGGTACCAAGACCTACACTTGCAACGTG |
| | | GACCACAAGCCTTCCAACACTAAGGTGGACAAGCGCGT |
| | | CGAATCGAAGTACGGCCCACCGTGCCCGCCTTGTCCCG |
| | | CGCCGGAGTTCCTCGGCGGTCCCTCGGTCTTTCTGTTCC |
| | | CACCGAAGCCCAAGGACACTTTGATGATTTCCCGCACC |
| | | CCTGAAGTGACATGCGTGGTCGTGGACGTGTCACAGGA |
| | | AGATCCGGAGGTGCAGTTCAATTGGTACGTGGATGGCG |
| | | TCGAGGTGCACAACGCCAAAACCAAGCCGAGGGAGGA |
| | | GCAGTTCAACTCCACTTACCGCGTCGTGTCCGTGCTGAC |
| | | GGTGCTGCATCAGGACTGGCTGAACGGGAAGGAGTACA |
| | | AGTGCAAAGTGTCCAACAAGGGACTTCCTAGCTCAATC |
| | | GAAAAGACCATCTCGAAAGCCAAGGGACAGCCCCGGG |
| | | AACCCCAAGTGTATACCCTGCCACCGAGCCAGGAAGAA |
| | | ATGACTAAGAACCAAGTCTCATTGACTTGCCTTGTGAAG |
| | | GGCTTCTACCCATCGGATATCGCCGTGGAATGGGAGTC |
| | | CAACGGCCAGCCGGAAAACAACTACAAGACCACCCCTC |
| | | CGGTGCTGGACTCAGACGGATCCTTCTTCCTCTACTCGC |
| | | GGCTGACCGTGGATAAGAGCAGATGGCAGGAGGGAAA |
| | | TGTGTTCAGCTGTTCTGTGATGCATGAAGCCCTGCACAA |
| | | CCACTACACTCAGAAGTCCCTGTCCCTCTCCCTGGGA |
| BAP058-Clone O LC | | |
| SEQ ID NO: 3056 | LCDR1 | KASQDVGTAVA |
| (Kabat) | | |
| SEQ ID NO: 3057 | LCDR2 | WASTRHT |
| (Kabat) | | |
| SEQ ID NO: 3058 | LCDR3 | QQYNSYPLT |
| (Kabat) | | |
| SEQ ID NO: 3059 | LCDR1 | SQDVGTA |
| (Chothia) | | |
| SEQ ID NO: | LCDR2 | WAS |
| 3060(Chothia) | | |
| SEQ ID NO: 3061 | LCDR3 | YNSYPL |
| (Chothia) | | |
| SEQ ID NO: 3063 | VL | AIQLTQSPSSLSASVGDRVTITCKASQDVGTAVAWYLQKP |
| | | GQSPQLLIYWASTRHTGVPSRFSGSGSGTDFTFTISSLEAED |
| | | AATYYCQQYNSYPLTFGQGTKVEIK |
| SEQ ID NO: 3064 | DNA | GCTATTCAGCTGACTCAGTCACCTAGTAGCCTGAGCGCT |
| | VL | AGTGTGGGCGATAGAGTGACTATCACCTGTAAAGCCTC |
| | | TCAGGACGTGGGCACCGCCGTGGCCTGGTATCTGCAGA |
| | | AGCCTGGTCAATCACCTCAGCTGCTGATCTACTGGGCCT |
| | | CTACTAGACACACCGGCGTGCCCTCTAGGTTTAGCGGTA |
| | | GCGGTAGTGGCACCGACTTCACCTTCACTATCTCTTCAC |
| | | TGGAAGCCGAGGACGCCGCTACCTACTACTGTCAGCAG |
| | | TATAATAGCTACCCCCTGACCTTCGGTCAAGGCACTAAG |
| | | GTCGAGATTAAG |
| SEQ ID NO: 3065 | Light | AIQLTQSPSSLSASVGDRVTITCKASQDVGTAVAWYLQKP |
| | chain | GQSPQLLIYWASTRHTGVPSRFSGSGSGTDFTFTISSLEAED |
| | | AATYYCQQYNSYPLTFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| SEQ ID NO: 3066 | DNA | GCTATTCAGCTGACTCAGTCACCTAGTAGCCTGAGCGCT |
| | light | AGTGTGGGCGATAGAGTGACTATCACCTGTAAAGCCTC |
| | chain | TCAGGACGTGGGCACCGCCGTGGCCTGGTATCTGCAGA |
| | | AGCCTGGTCAATCACCTCAGCTGCTGATCTACTGGGCCT |
| | | CTACTAGACACACCGGCGTGCCCTCTAGGTTTAGCGGTA |
| | | GCGGTAGTGGCACCGACTTCACCTTCACTATCTCTTCAC |
| | | TGGAAGCCGAGGACGCCGCTACCTACTACTGTCAGCAG |
| | | TATAATAGCTACCCCCTGACCTTCGGTCAAGGCACTAAG |
| | | GTCGAGATTAAGCGTACGGTGGCCGCTCCCAGCGTGTT |
| | | CATCTTCCCCCCCAGCGACGAGCAGCTGAAGAGCGGCA |
| | | CCGCCAGCGTGGTGTGCCTGCTGAACAACTTCTACCCCC |
| | | GGGAGGCCAAGGTGCAGTGGAAGGTGGACAACGCCCT |
| | | GCAGAGCGGCAACAGCCAGGAGAGCGTCACCGAGCAG |
| | | GACAGCAAGGACTCCACCTACAGCCTGAGCAGCACCCT |
| | | GACCCTGAGCAAGGCCGACTACGAGAAGCATAAGGTGT |
| | | ACGCCTGCGAGGTGACCCACCAGGGCCTGTCCAGCCCC |
| | | GTGACCAAGAGCTTCAACAGGGGCGAGTGC |
| BAP058-Clone N HC | | |
| SEQ ID NO: 3048 | HCDR1 | SYWMY |
| (Kabat) | | |
| SEQ ID NO: 3049 | HCDR2 | RIDPNSGSTKYNEKFKN |
| (Kabat) | | |
| SEQ ID NO: 3050 | HCDR3 | DYRKGLYAMDY |
| (Kabat) | | |
| SEQ ID NO: | HCDR1 | GYTFTSY |
| 3051(Chothia) | | |
| SEQ ID NO: | HCDR2 | DPNSGS |
| 3052(Chothia) | | |
| SEQ ID NO: 3050 | HCDR3 | DYRKGLYAMDY |
| (Chothia) | | |
| SEQ ID NO: 3067 | VH | EVQLVQSGAEVKKPGATVKISCKVSGYTFTSYWMYWVR |
| | | QATGQGLEWMGRIDPNSGSTKYNEKFKNRVTITADKSTST |
| | | AYMELSSLRSEDTAVYYCARDYRKGLYAMDYWGQGTTV |
| | | TVSS |
| SEQ ID NO: 3068 | DNA | GAAGTGCAGCTGGTGCAGTCAGGCGCCGAAGTGAAGAA |
| | VH | ACCCGGCGCTACCGTGAAGATTAGCTGTAAAGTCTCAG |
| | | GCTACACCTTCACTAGCTACTGGATGTACTGGGTCCGAC |
| | | AGGCTACCGGTCAAGGCCTGGAGTGGATGGGTAGAATC |
| | | GACCCTAATAGCGGCTCTACTAAGTATAACGAGAAGTT |
| | | TAAGAATAGAGTGACTATCACCGCCGATAAGTCTACTA |
| | | GCACCGCCTATATGGAACTGTCTAGCCTGAGATCAGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCA |
| SEQ ID NO: 3069 | Heavy | EVQLVQSGAEVKKPGATVKISCKVSGYTFTSYWMYWVR |
| | chain | QATGQGLEWMGRIDPNSGSTKYNEKFKNRVTITADKSTST |
| | | AYMELSSLRSEDTAVYYCARDYRKGLYAMDYWGQGTTV |
| | | TVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFFLYSRLTVDKSRWQEGNVFSCSVMHEALHNHY |
| | | TQKSLSLSLG |
| SEQ ID NO: 3070 | DNA | GAAGTGCAGCTGGTGCAGTCAGGCGCCGAAGTGAAGAA |
| | heavy | ACCCGGCGCTACCGTGAAGATTAGCTGTAAAGTCTCAG |
| | chain | GCTACACCTTCACTAGCTACTGGATGTACTGGGTCCGAC |
| | | AGGCTACCGGTCAAGGCCTGGAGTGGATGGGTAGAATC |
| | | GACCCTAATAGCGGCTCTACTAAGTATAACGAGAAGTT |
| | | TAAGAATAGAGTGACTATCACCGCCGATAAGTCTACTA |
| | | GCACCGCCTATATGGAACTGTCTAGCCTGAGATCAGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCAGCTAGCACTAAGGGCCCGTCCG |
| | | TGTTCCCCCTGGCACCTTGTAGCCGGAGCACTAGCGAAT |
| | | CCACCGCTGCCCTCGGCTGCCTGGTCAAGGATTACTTCC |
| | | CGGAGCCCGTGACCGTGTCCTGGAACAGCGGAGCCCTG |
| | | ACCTCCGGAGTGCACACCTTCCCCGCTGTGCTGCAGAGC |
| | | TCCGGGCTGTACTCGCTGTCGTCGGTGGTCACGGTGCCT |
| | | TCATCTAGCCTGGGTACCAAGACCTACACTTGCAACGTG |
| | | GACCACAAGCCTTCCAACACTAAGGTGGACAAGCGCGT |
| | | CGAATCGAAGTACGGCCCACCGTGCCCGCCTTGTCCCG |
| | | CGCCGGAGTTCCTCGGCGGTCCCTCGGTCTTTCTGTTCC |
| | | CACCGAAGCCCAAGGACACTTTGATGATTTCCCGCACC |
| | | CCTGAAGTGACATGCGTGGTCGTGGACGTGTCACAGGA |
| | | AGATCCGGAGGTGCAGTTCAATTGGTACGTGGATGGCG |
| | | TCGAGGTGCACAACGCCAAAACCAAGCCGAGGGAGGA |
| | | GCAGTTCAACTCCACTTACCGCGTCGTGTCCGTGCTGAC |
| | | GGTGCTGCATCAGGACTGGCTGAACGGGAAGGAGTACA |
| | | AGTGCAAAGTGTCCAACAAGGGACTTCCTAGCTCAATC |
| | | GAAAAGACCATCTCGAAAGCCAAGGGACAGCCCCGGG |
| | | AACCCCAAGTGTATACCCTGCCACCGAGCCAGGAAGAA |
| | | ATGACTAAGAACCAAGTCTCATTGACTTGCCTTGTGAAG |
| | | GGCTTCTACCCATCGGATATCGCCGTGGAATGGGAGTC |
| | | CAACGGCCAGCCGGAAAACAACTACAAGACCACCCCTC |
| | | CGGTGCTGGACTCAGACGGATCCTTCTTCCTCTACTCGC |
| | | GGCTGACCGTGGATAAGAGCAGATGGCAGGAGGGAAA |
| | | TGTGTTCAGCTGTTCTGTGATGCATGAAGCCCTGCACAA |
| | | CCACTACACTCAGAAGTCCCTGTCCCTCTCCCTGGGA |
| BAP058-Clone N LC | | |
| SEQ ID NO: 3056 | LCDR1 | KASQDVGTAVA |
| (Kabat) | | |
| SEQ ID NO: 3057 | LCDR2 | WASTRHT |
| (Kabat) | | |
| SEQ ID NO: 3058 | LCDR3 | QQYNSYPLT |
| (Kabat) | | |
| SEQ ID NO: 3059 | LCDR1 | SQDVGTA |
| (Chothia) | | |
| SEQ ID NO: | LCDR2 | WAS |
| 3060(Chothia) | | |
| SEQ ID NO: 3061 | LCDR3 | YNSYPL |
| (Chothia) | | |
| SEQ ID NO: 3071 | VL | DVVMTQSPLSLPVTLGQPASISCKASQDVGTAVAWYQQK |
| | | PGQAPRLLIYWASTRHTGVPSRFSGSGSGTEFTLTISSLQPD |
| | | DFATYYCQQYNSYPLTFGQGTKVEIK |
| SEQ ID NO: 3072 | DNA | GACGTCGTGATGACTCAGTCACCCCTGAGCCTGCCCGTG |
| | VL | ACCCTGGGGCAGCCCGCCTCTATTAGCTGTAAAGCCTCT |
| | | CAGGACGTGGGCACCGCCGTGGCCTGGTATCAGCAGAA |
| | | GCCAGGGCAAGCCCCTAGACTGCTGATCTACTGGGCCT |
| | | CTACTAGACACACCGGCGTGCCCTCTAGGTTTAGCGGTA |
| | | GCGGTAGTGGCACCGAGTTCACCCTGACTATCTCTTCAC |
| | | TGCAGCCCGACGACTTCGCTACCTACTACTGTCAGCAGT |
| | | ATAATAGCTACCCCCTGACCTTCGGTCAAGGCACTAAG |
| | | GTCGAGATTAAG |
| SEQ ID NO: 3073 | Light | DVVMTQSPLSLPVTLGQPASISCKASQDVGTAVAWYQQK |
| | chain | PGQAPRLLIYWASTRHTGVPSRFSGSGSGTEFTLTISSLQPD |
| | | DFATYYCQQYNSYPLTFGQGTKVEIKRTVAAPSVFIFPPSD |
| | | EQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |
| SEQ ID NO: 3074 | DNA | GACGTCGTGATGACTCAGTCACCCCTGAGCCTGCCCGTG |
| | light | ACCCTGGGGCAGCCCGCCTCTATTAGCTGTAAAGCCTCT |
| | chain | CAGGACGTGGGCACCGCCGTGGCCTGGTATCAGCAGAA |
| | | GCCAGGGCAAGCCCCTAGACTGCTGATCTACTGGGCCT |
| | | CTACTAGACACACCGGCGTGCCCTCTAGGTTTAGCGGTA |
| | | GCGGTAGTGGCACCGAGTTCACCCTGACTATCTCTTCAC |
| | | TGCAGCCCGACGACTTCGCTACCTACTACTGTCAGCAGT |
| | | ATAATAGCTACCCCCTGACCTTCGGTCAAGGCACTAAG |
| | | GTCGAGATTAAGCGTACGGTGGCCGCTCCCAGCGTGTT |
| | | CATCTTCCCCCCCAGCGACGAGCAGCTGAAGAGCGGCA |
| | | CCGCCAGCGTGGTGTGCCTGCTGAACAACTTCTACCCCC |
| | | GGGAGGCCAAGGTGCAGTGGAAGGTGGACAACGCCCT |
| | | GCAGAGCGGCAACAGCCAGGAGAGCGTCACCGAGCAG |
| | | GACAGCAAGGACTCCACCTACAGCCTGAGCAGCACCCT |
| | | GACCCTGAGCAAGGCCGACTACGAGAAGCATAAGGTGT |
| | | ACGCCTGCGAGGTGACCCACCAGGGCCTGTCCAGCCCC |
| | | GTGACCAAGAGCTTCAACAGGGGCGAGTGC |
| BAP058-Clone O HC | | |
| SEQ ID NO: 3075 | HCDR1 | agctactggatgtac |
| (Kabat) | | |
| SEQ ID NO: 3076 | HCDR2 | agaatcgaccctaatagcggctctactaagtataacgagaagtttaagaat |
| (Kabat) | | |
| SEQ ID NO: 3077 | HCDR3 | gactatagaaagggcctgtacgctatggactac |
| (Kabat) | | |
| SEQ ID NO: 3078 | HCDR1 | ggctacaccttcactagctac |
| (Chothia) | | |
| SEQ ID NO: 3079 | HCDR2 | gaccctaatagcggctct |
| (Chothia) | | |
| SEQ ID NO: 3077 | HCDR3 | gactatagaaagggcctgtacgctatggactac |
| (Chothia) | | |
| BAP058-Clone O LC | | |
| SEQ ID NO: 3080 | LCDR1 | aaagcctctcaggacgtgggcaccgccgtggcc |
| (Kabat) | | |
| SEQ ID NO: 3081 | LCDR2 | tgggcctctactagacacacc |
| (Kabat) | | |
| SEQ ID NO: 3082 | LCDR3 | cagcagtataatagctaccccctgacc |
| (Kabat) | | |
| SEQ ID NO: | LCDR1 | tctcaggacgtgggcaccgcc |
| 3083(Chothia) | | |
| SEQ ID NO: 3084 | LCDR2 | tgggcctct |
| (Chothia) | | |
| SEQ ID NO: | LCDR3 | tataatagctaccccctg |
| 3085(Chothia) | | |
| BAP058-Clone N HC | | |
| SEQ ID NO: 3075 | HCDR1 | agctactggatgtac |
| (Kabat) | | |
| SEQ ID NO: 3076 | HCDR2 | agaatcgaccctaatagcggctctactaagtataacgagaagtttaagaat |
| (Kabat) | | |
| SEQ ID NO: 3077 | HCDR3 | gactatagaaagggcctgtacgctatggactac |
| (Kabat) | | |
| SEQ ID NO: 3078 | HCDR1 | ggctacaccttcactagctac |
| (Chothia) | | |
| SEQ ID NO: 3079 | HCDR2 | gaccctaatagcggctct |
| (Chothia) | | |
| SEQ ID NO: 3077 | HCDR3 | gactatagaaagggcctgtacgctatggactac |
| (Chothia) | | |
| BAP058-Clone N LC | | |
| SEQ ID NO: 3080 | LCDR1 | aaagcctctcaggacgtgggcaccgccgtggcc |
| (Kabat) | | |
| SEQ ID NO: 3081 | LCDR2 | tgggcctctactagacacacc |
| (Kabat) | | |
| SEQ ID NO: 3082 | LCDR3 | cagcagtataatagctaccccctgacc |
| (Kabat) | | |
| SEQ ID NO: 3083 | LCDR1 | tctcaggacgtgggcaccgcc |
| (Chothia) | | |
| SEQ ID NO: 3084 | LCDR2 | tgggcctct |
| (Chothia) | | |
| SEQ ID NO: 3085 | LCDR3 | tataatagctaccccctg |
| (Chothia) | | |

### Other Exemplary PD-L1 Inhibitors

In some embodiments, the PD-L1 inhibitor is anti-PD-L1 antibody. In some embodiments, the anti-PD-L1 inhibitor is selected from YW243.55.S70, MPDL3280A, MEDI-4736, or MDX-1105MSB-0010718C (also referred to as A09-246-2) disclosed in, e.g., WO 2013/0179174, and having a sequence disclosed herein (or a sequence substantially identical or similar thereto, e.g., a sequence at least 85%, 90%, 95% identical or higher to the sequence specified).

In one embodiment, the PD-L1 inhibitor is MDX-1105. MDX-1105, also known as BMS-936559, is an anti-PD-L1 antibody described in PCT Publication No. WO 2007/005874.

In one embodiment, the PD-L1 inhibitor is YW243.55.S70. The YW243.55.S70 antibody is an anti-PD-L1 described in PCT Publication No. WO 2010/077634.

In one embodiment, the PD-L1 inhibitor is MDPL3280A (Genentech/Roche) also known as Atezolizumabm, RG7446, R05541267, YW243.55.S70, or TECENTRIQ™. MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906 incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab, e.g., as disclosed in Table 17.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in PCT Publication Nos. WO2010/027827 and WO2011/066342).

In one embodiment, the PD-L1 inhibitor is an anti-PD-L1 antibody molecule. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 17.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 17.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 17.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 17
Amino acid sequences of other exemplary anti-PD-L1 antibody molecules

| | | |
|---|---|---|
| Atezolizumab | | |
| SEQ ID NO: | Heavy | EVQLVESGGGLVQPGGSLRLSCAASGFTFSDSWIHWVRQAPGKGLE |
| 3086 | chain | WVAWISPYGGSTYYADSVKGRFTISADTSKNTAYLQMNSLRAEDTA |
| | | VYYCARRHWPGGFDYWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYASTYRVVSVLTVLHQDWLNGKEYKCK |
| | | VSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| SEQ ID NO: | Light | DIQMTQSPSSLSASVGDRVTITCRASQDVSTAVAWYQQKPGKAPKL |
| 3087 | chain | LIYSASFLYSGVPSRFSGSGSGTDFTLTISSLQPEDFATYYCQQYLYHP |
| | | ATFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKH |
| | | KVYACEVTHQGLSSPVTKSFNRGEC |
| Avelumab | | |
| SEQ ID NO: | Heavy | EVQLLESGGGLVQPGGSLRLSCAASGFTFSSYIMMWVRQAPGKGLE |
| 3088 | chain | WVSSIYPSGGITFYADTVKGRFTISRDNSKNTLYLQMNSLRAEDTAV |
| | | YYCARIKLGTVTTVDYWGQGTLVTVSSASTKGPSVFPLAPSSKSTSG |
| | | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| SEQ ID NO: | Light | QSALTQPASVSGSPGQSITISCTGTSSDVGGYNYVSWYQQHPGKAPK |
| 3089 | chain | LMIYDVSNRPSGVSNRFSGSKSGNTASLTISGLQAEDEADYYCSSYTS |
| | | SSTRVFGTGTKVTVLGQPKANPTVTLFPPSSEELQANKATLVCLISDF |
| | | YPGAVTVAWKADGSPVKAGVETTKPSKQSNNKYAASSYLSLTPEQ |
| | | WKSHRSYSCQVTHEGSTVEKTVAPTECS |
| Durvalumab | | |
| SEQ ID NO: | Heavy | EVQLVESGGGLVQPGGSLRLSCAASGFTFSRYWMSWVRQAPGKGL |
| 3090 | chain | EWVANIKQDGSEKYYVDSVKGRFTISRDNAKNSLYLQMNSLRAEDT |
| | | AVYYCAREGGWFGELAFDYWGQGTLVTVSSASTKGPSVFPLAPSSK |
| | | STSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPP |
| | | CPAPEFEGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPASIEKTISKAKGQPREPQVYTLPPSREEMTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| SEQ ID NO: | Light | EIVLTQSPGTLSLSPGERATLSCRASQRVSSSYLAWYQQKPGQAPRLL |
| 3091 | chain | IYDASSRATGIPDRFSGSGSGTDFTLTISRLEPEDFAVYYCQQYGSLP |
| | | WTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKH |
| | | KVYACEVTHQGLSSPVTKSFNRGEC |
| BMS-936559 | | |
| SEQ ID NO: | VH | QVQLVQSGAEVKKPGSSVKVSCKTSGDTFSTYAISWVRQAPGQGLE |
| 3092 | | WMGGIIPIFGKAHYAQKFQGRVTITADESTSTAYMELSSLRSEDTAV |
| | | YFCARKFHFVSGSPFGMDVWGQGTTVTVSS |
| SEQ ID NO: | VL | EIVLTQSPATLSLSPGERATLSCRASQSVSSYLAWYQQKPGQAPRLLI |
| 3093 | | YDASNRATGIPARFSGSGSGTDFTLTISSLEPEDFAVYYCQQRSNWPT |
| | | FGQGTKVEIK |

### LAG-3 Inhibitors

In some embodiments, the ZBTB32 inhibitor, or a pharmaceutically acceptable salt, hydrate, solvate, prodrug, stereoisomer, or tautomer thereof, of the present disclosure are used in combination with a LAG-3 inhibitor to treat a disease, e.g., cancer. In some embodiments, the LAG-3 inhibitor is selected from LAG525 (Novartis), BMS-986016 (Bristol-Myers Squibb), or TSR-033 (Tesaro).

Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 18 (e.g., from the heavy and light chain variable region sequences of BAP050-Clone I or BAP050-Clone J disclosed in Table 18), or encoded by a nucleotide sequence shown in Table 18. In some embodiments, the CDRs are according to the Kabat definition (e.g., as set out in Table 18). In some embodiments, the CDRs are according to the Chothia definition (e.g., as set out in Table 18). In some embodiments, the CDRs are according to the combined CDR definitions of both Kabat and Chothia (e.g., as set out in Table 18). In one embodiment, the combination of Kabat and Chothia CDR of VH CDR1 comprises the amino acid sequence GFTLTNYGMN (SEQ ID NO: 3158). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 18, or encoded by a nucleotide sequence shown in Table 18.

In one embodiment, the anti-LAG-3 antibody molecule comprises a heavy chain variable region (VH) comprising a VHCDR1 amino acid sequence of SEQ ID NO: 3094, a VHCDR2 amino acid sequence of SEQ ID NO: 3095, and a VHCDR3 amino acid sequence of SEQ ID NO: 3096; and a light chain variable region (VL) comprising a VLCDR1 amino acid sequence of SEQ ID NO: 3103, a VLCDR2 amino acid sequence of SEQ ID NO: 3104, and a VLCDR3 amino acid sequence of SEQ ID NO: 3105, each disclosed in Table 18.

In one embodiment, the anti-LAG-3 antibody molecule comprises a VH comprising a VHCDR1 encoded by the nucleotide sequence of SEQ ID NO: 3129 or 3130, a VHCDR2 encoded by the nucleotide sequence of SEQ ID NO: 3131 or 3132, and a VHCDR3 encoded by the nucleotide sequence of SEQ ID NO: 3133 or 3134; and a VL comprising a VLCDR1 encoded by the nucleotide sequence of SEQ ID NO: 3139 or 3140, a VLCDR2 encoded by the nucleotide sequence of SEQ ID NO: 3141 or 3142, and a VLCDR3 encoded by the nucleotide sequence of SEQ ID NO: 3143 or 3144, each disclosed in Table 18. In one embodiment, the anti-LAG-3 antibody molecule comprises a VH comprising a VHCDR1 encoded by the nucleotide sequence of SEQ ID NO: 3150 or 3130, a VHCDR2 encoded by the nucleotide sequence of SEQ ID NO: 3151 or 3132, and a VHCDR3 encoded by the nucleotide sequence of SEQ ID NO: 3152 or 3134; and a VL comprising a VLCDR1 encoded by the nucleotide sequence of SEQ ID NO: 3139 or 3140, a VLCDR2 encoded by the nucleotide sequence of SEQ ID NO: 3141 or 3142, and a VLCDR3 encoded by the nucleotide sequence of SEQ ID NO: 3143 or 3144, each disclosed in Table 18.

In one embodiment, the anti-LAG-3 antibody molecule comprises a VH comprising the amino acid sequence of SEQ ID NO: 3099, or an amino acid sequence at least 85%, 90%, 95%, or 99% identical or higher to SEQ ID NO: 3099. In one embodiment, the anti-LAG-3 antibody molecule comprises a VL comprising the amino acid sequence of SEQ ID NO: 3111, or an amino acid sequence at least 85%, 90%, 95%, or 99% identical or higher to SEQ ID NO: 3111. In one embodiment, the anti-LAG-3 antibody molecule comprises a VH comprising the amino acid sequence of SEQ ID NO: 3117, or an amino acid sequence at least 85%, 90%, 95%, or 99% identical or higher to SEQ ID NO: 3117. In one embodiment, the anti-LAG-3 antibody molecule comprises a VL comprising the amino acid sequence of SEQ ID NO: 3123, or an amino acid sequence at least 85%, 90%, 95%, or 99% identical or higher to SEQ ID NO: 3123. In one embodiment, the anti-LAG-3 antibody molecule comprises a VH comprising the amino acid sequence of SEQ ID NO: 3099 and a VL comprising the amino acid sequence of SEQ ID NO: 3111. In one embodiment, the anti-LAG-3 antibody molecule comprises a VH comprising the amino acid sequence of SEQ ID NO: 3117 and a VL comprising the amino acid sequence of SEQ ID NO: 3123.

In one embodiment, the antibody molecule comprises a VH encoded by the nucleotide sequence of SEQ ID NO: 3100 or 3101, or a nucleotide sequence at least 85%, 90%, 95%, or 99% identical or higher to SEQ ID NO: 3100 or 3101. In one embodiment, the antibody molecule comprises a VL encoded by the nucleotide sequence of SEQ ID NO: 3112 or 3113, or a nucleotide sequence at least 85%, 90%, 95%, or 99% identical or higher to SEQ ID NO: 3112 or 3113. In one embodiment, the antibody molecule comprises a VH encoded by the nucleotide sequence of SEQ ID NO: 3118 or 3119, or a nucleotide sequence at least 85%, 90%, 95%, or 99% identical or higher to SEQ ID NO: 3118 or 3119. In one embodiment, the antibody molecule comprises a VL encoded by the nucleotide sequence of SEQ ID NO: 3124 or 3125, or a nucleotide sequence at least 85%, 90%, 95%, or 99% identical or higher to SEQ ID NO: 3124 or 3125. In one embodiment, the antibody molecule comprises a VH encoded by the nucleotide sequence of SEQ ID NO: 3100 or 3101 and a VL encoded by the nucleotide sequence of SEQ ID NO: 3112 or 3113. In one embodiment, the antibody molecule comprises a VH encoded by the nucleotide sequence of SEQ ID NO: 3118 or 3119 and a VL encoded by the nucleotide sequence of SEQ ID NO: 3124 or 3125.

In one embodiment, the anti-LAG-3 antibody molecule comprises a heavy chain comprising the amino acid sequence of SEQ ID NO: 3102, or an amino acid sequence at least 85%, 90%, 95%, or 99% identical or higher to SEQ ID NO: 3102. In one embodiment, the anti-LAG-3 antibody molecule comprises a light chain comprising the amino acid sequence of SEQ ID NO: 3114, or an amino acid sequence at least 85%, 90%, 95%, or 99% identical or higher to SEQ ID NO: 3114. In one embodiment, the anti-LAG-3 antibody molecule comprises a heavy chain comprising the amino acid sequence of SEQ ID NO: 3120, or an amino acid sequence at least 85%, 90%, 95%, or 99% identical or higher to SEQ ID NO: 3120. In one embodiment, the anti-LAG-3 antibody molecule comprises a light chain comprising the amino acid sequence of SEQ ID NO: 3126, or an amino acid sequence at least 85%, 90%, 95%, or 99% identical or higher to SEQ ID NO: 3126. In one embodiment, the anti-LAG-3 antibody molecule comprises a heavy chain comprising the amino acid sequence of SEQ ID NO: 3102 and a light chain comprising the amino acid sequence of SEQ ID NO: 3114. In one embodiment, the anti-LAG-3 antibody molecule comprises a heavy chain comprising the amino acid sequence of SEQ ID NO: 3120 and a light chain comprising the amino acid sequence of SEQ ID NO: 3126.

In one embodiment, the antibody molecule comprises a heavy chain encoded by the nucleotide sequence of SEQ ID NO: 3109 or 3110, or a nucleotide sequence at least 85%, 90%, 95%, or 99% identical or higher to SEQ ID NO: 3109 or 3110. In one embodiment, the antibody molecule comprises a light chain encoded by the nucleotide sequence of SEQ ID NO: 3115 or 3116, or a nucleotide sequence at least 85%, 90%, 95%, or 99% identical or higher to SEQ ID NO: 3115 or 3116. In one embodiment, the antibody molecule comprises a heavy chain encoded by the nucleotide sequence of SEQ ID NO: 3121 or 3122, or a nucleotide sequence at least 85%, 90%, 95%, or 99% identical or higher to SEQ ID NO: 3121 or 3122. In one embodiment, the antibody molecule comprises a light chain encoded by the nucleotide sequence of SEQ ID NO: 3127 or 3128, or a nucleotide sequence at least 85%, 90%, 95%, or 99% identical or higher to SEQ ID NO: 3127 or 3128. In one embodiment, the antibody molecule comprises a heavy chain encoded by the nucleotide sequence of SEQ ID NO: 3109 or 3110 and a light chain encoded by the nucleotide sequence of SEQ ID NO: 3115 or 3116. In one embodiment, the antibody molecule comprises a heavy chain encoded by the nucleotide sequence of SEQ ID NO: 3121 or 3122 and a light chain encoded by the nucleotide sequence of SEQ ID NO: 3127 or 3128.

The antibody molecules described herein can be made by vectors, host cells, and methods described in US 2015/0259420, incorporated by reference in its entirety.

TABLE 18
Amino acid and nucleotide sequences of exemplary
anti-LAG-3 antibody molecules

| | | |
|---|---|---|
| BAP050-Clone I HC | | |
| SEQ ID NO: 3094 | HCDR1 | NYGMN |
| (Kabat) | | |
| SEQ ID NO: 3095 | HCDR2 | WINTDTGEPTYADDFKG |
| (Kabat) | | |
| SEQ ID NO: 3096 | HCDR3 | NPPYYYGTNNAEAMDY |
| (Kabat) | | |
| SEQ ID NO: 3097 | HCDR1 | GFTLTNY |
| (Chothia) | | |
| SEQ ID NO: | HCDR2 | NTDTGE |
| 3098(Chothia) | | |
| SEQ ID NO: 3096 | HCDR3 | NPPYYYGTNNAEAMDY |
| (Chothia) | | |
| SEQ ID NO: 3099 | VH | QVQLVQSGAEVKKPGASVKVSCKASGFTLTNYGMNWVRQAR |
| | | GQRLEWIGWINTDTGEPTYADDFKGRFVFSLDTSVSTAYLQISS |
| | | LKAEDTAVYYCARNPPYYYGTNNAEAMDYWGQGTTVTVSS |
| SEQ ID NO: 3100 | DNA | CAAGTGCAGCTGGTGCAGTCGGGAGCCGAAGTGAAGAAGCC |
| | VH | TGGAGCCTCGGTGAAGGTGTCGTGCAAGGCATCCGGATTCA |
| | | CCCTCACCAATTACGGGATGAACTGGGTCAGACAGGCCCGG |
| | | GGTCAACGGCTGGAGTGGATCGGATGGATTAACACCGACAC |
| | | CGGGGAGCCTACCTACGCGGACGATTTCAAGGGACGGTTCG |
| | | TGTTCTCCCTCGACACCTCCGTGTCCACCGCCTACCTCCAAA |
| | | TCTCCTCACTGAAAGCGGAGGACACCGCCGTGTACTATTGC |
| | | GCGAGGAACCCGCCCTACTACTACGGAACCAACAACGCCGA |
| | | AGCCATGGACTACTGGGGCCAGGGCACCACTGTGACTGTGT |
| | | CCAGC |
| SEQ ID NO: 3101 | DNA | CAGGTGCAGCTGGTGCAGTCTGGCGCCGAAGTGAAGAAACC |
| | VH | TGGCGCCTCCGTGAAGGTGTCCTGCAAGGCCTCTGGCTTCAC |
| | | CCTGACCAACTACGGCATGAACTGGGTGCGACAGGCCAGGG |
| | | GCCAGCGGCTGGAATGGATCGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCT |
| SEQ ID NO: 3102 | Heavy | QVQLVQSGAEVKKPGASVKVSCKASGFTLTNYGMNWVRQAR |
| | chain | GQRLEWIGWINTDTGEPTYADDFKGRFVFSLDTSVSTAYLQISS |
| | | LKAEDTAVYYCARNPPYYYGTNNAEAMDYWGQGTTVTVSSA |
| | | STKGPSVFPLAPCSRSTSESTAALGCLVKDYFPEPVTVSWNSGA |
| | | LTSGVHTFPAVLQSSGLYSLSSVVTVPSSSLGTKTYTCNVDHKP |
| | | SNTKVDKRVESKYGPPCPPCPAPEFLGGPSVFLFPPKPKDTLMIS |
| | | RTPEVTCVVVDVSQEDPEVQFNWYVDGVEVHNAKTKPREEQF |
| | | NSTYRVVSVLTVLHQDWLNGKEYKCKVSNKGLPSSIEKTISKA |
| | | KGQPREPQVYTLPPSQEEMTKNQVSLTCLVKGFYPSDIAVEWE |
| | | SNGQPENNYKTTPPVLDSDGSFFLYSRLTVDKSRWQEGNVFSC |
| | | SVMHEALHNHYTQKSLSLSLG |
| SEQ ID NO: 3109 | DNA | CAAGTGCAGCTGGTGCAGTCGGGAGCCGAAGTGAAGAAGCC |
| | heavy | TGGAGCCTCGGTGAAGGTGTCGTGCAAGGCATCCGGATTCA |
| | chain | CCCTCACCAATTACGGGATGAACTGGGTCAGACAGGCCCGG |
| | | GGTCAACGGCTGGAGTGGATCGGATGGATTAACACCGACAC |
| | | CGGGGAGCCTACCTACGCGGACGATTTCAAGGGACGGTTCG |
| | | TGTTCTCCCTCGACACCTCCGTGTCCACCGCCTACCTCCAAA |
| | | TCTCCTCACTGAAAGCGGAGGACACCGCCGTGTACTATTGC |
| | | GCGAGGAACCCGCCCTACTACTACGGAACCAACAACGCCGA |
| | | AGCCATGGACTACTGGGGCCAGGGCACCACTGTGACTGTGT |
| | | CCAGCGCGTCCACTAAGGGCCCGTCCGTGTTCCCCCTGGCAC |
| | | CTTGTAGCCGGAGCACTAGCGAATCCACCGCTGCCCTCGGCT |
| | | GCCTGGTCAAGGATTACTTCCCGGAGCCCGTGACCGTGTCCT |
| | | GGAACAGCGGAGCCCTGACCTCCGGAGTGCACACCTTCCCC |
| | | GCTGTGCTGCAGAGCTCCGGGCTGTACTCGCTGTCGTCGGTG |
| | | GTCACGGTGCCTTCATCTAGCCTGGGTACCAAGACCTACACT |
| | | TGCAACGTGGACCACAAGCCTTCCAACACTAAGGTGGACAA |
| | | GCGCGTCGAATCGAAGTACGGCCCACCGTGCCCGCCTTGTC |
| | | CCGCGCCGGAGTTCCTCGGCGGTCCCTCGGTCTTTCTGTTCC |
| | | CACCGAAGCCCAAGGACACTTTGATGATTTCCCGCACCCCTG |
| | | AAGTGACATGCGTGGTCGTGGACGTGTCACAGGAAGATCCG |
| | | GAGGTGCAGTTCAATTGGTACGTGGATGGCGTCGAGGTGCA |
| | | CAACGCCAAAACCAAGCCGAGGGAGGAGCAGTTCAACTCCA |
| | | CTTACCGCGTCGTGTCCGTGCTGACGGTGCTGCATCAGGACT |
| | | GGCTGAACGGGAAGGAGTACAAGTGCAAAGTGTCCAACAA |
| | | GGGACTTCCTAGCTCAATCGAAAAGACCATCTCGAAAGCCA |
| | | AGGGACAGCCCCGGGAACCCCAAGTGTATACCCTGCCACCG |
| | | AGCCAGGAAGAAATGACTAAGAACCAAGTCTCATTGACTTG |
| | | CCTTGTGAAGGGCTTCTACCCATCGGATATCGCCGTGGAATG |
| | | GGAGTCCAACGGCCAGCCGGAAAACAACTACAAGACCACCC |
| | | CTCCGGTGCTGGACTCAGACGGATCCTTCTTCCTCTACTCGC |
| | | GGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 3110 | DNA heavy | CAGGTGCAGCTGGTGCAGTCTGGCGCCGAAGTGAAGAAACC |
| | chain | TGGCGCCTCCGTGAAGGTGTCCTGCAAGGCCTCTGGCTTCAC |
| | | CCTGACCAACTACGGCATGAACTGGGTGCGACAGGCCAGGG |
| | | GCCAGCGGCTGGAATGGATCGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCTGCTTCTACCAAGGGGCCCAGCGTGTTCCCCCTGGCCCC |
| | | CTGCTCCAGAAGCACCAGCGAGAGCACAGCCGCCCTGGGCT |
| | | GCCTGGTGAAGGACTACTTCCCCGAGCCCGTGACCGTGTCCT |
| | | GGAACAGCGGAGCCCTGACCAGCGGCGTGCACACCTTCCCC |
| | | GCCGTGCTGCAGAGCAGCGGCCTGTACAGCCTGAGCAGCGT |
| | | GGTGACCGTGCCCAGCAGCAGCCTGGGCACCAAGACCTACA |
| | | CCTGTAACGTGGACCACAAGCCCAGCAACACCAAGGTGGAC |
| | | AAGAGGGTGGAGAGCAAGTACGGCCCACCCTGCCCCCCCTG |
| | | CCCAGCCCCCGAGTTCCTGGGCGGACCCAGCGTGTTCCTGTT |
| | | CCCCCCCAAGCCCAAGGACACCCTGATGATCAGCAGAACCC |
| | | CCGAGGTGACCTGTGTGGTGGTGGACGTGTCCCAGGAGGAC |
| | | CCCGAGGTCCAGTTCAACTGGTACGTGGACGGCGTGGAGGT |
| | | GCACAACGCCAAGACCAAGCCCAGAGAGGAGCAGTTTAAC |
| | | AGCACCTACCGGGTGGTGTCCGTGCTGACCGTGCTGCACCA |
| | | GGACTGGCTGAACGGCAAAGAGTACAAGTGTAAGGTCTCCA |
| | | ACAAGGGCCTGCCAAGCAGCATCGAAAAGACCATCAGCAA |
| | | GGCCAAGGGCCAGCCTAGAGAGCCCCAGGTCTACACCCTGC |
| | | CACCCAGCCAAGAGGAGATGACCAAGAACCAGGTGTCCCTG |
| | | ACCTGTCTGGTGAAGGGCTTCTACCCAAGCGACATCGCCGT |
| | | GGAGTGGGAGAGCAACGGCCAGCCCGAGAACAACTACAAG |
| | | ACCACCCCCCCAGTGCTGGACAGCGACGGCAGCTTCTTCCTG |
| | | TACAGCAGGCTGACCGTGGACAAGTCCAGATGGCAGGAGGG |
| | | CAACGTCTTTAGCTGCTCCGTGATGCACGAGGCCCTGCACAA |
| | | CCACTACACCCAGAAGAGCCTGAGCCTGTCCCTGGGC |
| BAP050-Clone I LC | | |
| SEQ ID NO: 3103 | LCDR1 | SSSQDISNYLN |
| (Kabat) | | |
| SEQ ID NO: 3104 | LCDR2 | YTSTLHL |
| (Kabat) | | |
| SEQ ID NO: | LCDR3 | QQYYNLPWT |
| 3105(Kabat) | | |
| SEQ ID NO: 3106 | LCDR1 | SQDISNY |
| (Chothia) | | |
| SEQ ID NO: | LCDR2 | YTS |
| 3107(Chothia) | | |
| SEQ ID NO: 3108 | LCDR3 | YYNLPW |
| (Chothia) | | |
| SEQ ID NO: 3111 | VL | DIQMTQSPSSLSASVGDRVTITCSSSQDISNYLNWYLQKPGQSP |
| | | QLLIYYTSTLHLGVPSRFSGSGSGTEFTLTISSLQPDDFATYYCQ |
| | | QYYNLPWTFGQGTKVEIK |
| SEQ ID NO: 3112 | DNA | GATATTCAGATGACTCAGTCACCTAGTAGCCTGAGCGCTAGT |
| | VL | GTGGGCGATAGAGTGACTATCACCTGTAGCTCTAGTCAGGA |
| | | TATCTCTAACTACCTGAACTGGTATCTGCAGAAGCCCGGTCA |
| | | ATCACCTCAGCTGCTGATCTACTACACTAGCACCCTGCACCT |
| | | GGGCGTGCCCTCTAGGTTTAGCGGTAGCGGTAGTGGCACCG |
| | | AGTTCACCCTGACTATCTCTAGCCTGCAGCCCGACGACTTCG |
| | | CTACCTACTACTGTCAGCAGTACTATAACCTGCCCTGGACCT |
| | | TCGGTCAAGGCACTAAGGTCGAGATTAAG |
| SEQ ID NO: 3113 | DNA | GACATCCAGATGACCCAGTCCCCCTCCAGCCTGTCTGCTTCC |
| | VL | GTGGGCGACAGAGTGACCATCACCTGTTCCTCCAGCCAGGA |
| | | CATCTCCAACTACCTGAACTGGTATCTGCAGAAGCCCGGCC |
| | | AGTCCCCTCAGCTGCTGATCTACTACACCTCCACCCTGCACC |
| | | TGGGCGTGCCCTCCAGATTTTCCGGCTCTGGCTCTGGCACCG |
| | | AGTTTACCCTGACCATCAGCTCCCTGCAGCCCGACGACTTCG |
| | | CCACCTACTACTGCCAGCAGTACTACAACCTGCCCTGGACCT |
| | | TCGGCCAGGGCACCAAGGTGGAAATCAAG |
| SEQ ID NO: 3114 | Light | DIQMTQSPSSLSASVGDRVTITCSSSQDISNYLNWYLQKPGQSP |
| | chain | QLLIYYTSTLHLGVPSRFSGSGSGTEFTLTISSLQPDDFATYYCQ |
| | | QYYNLPWTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 3115 | DNA | GATATTCAGATGACTCAGTCACCTAGTAGCCTGAGCGCTAGT |
| | light | GTGGGCGATAGAGTGACTATCACCTGTAGCTCTAGTCAGGA |
| | chain | TATCTCTAACTACCTGAACTGGTATCTGCAGAAGCCCGGTCA |
| | | ATCACCTCAGCTGCTGATCTACTACACTAGCACCCTGCACCT |
| | | GGGCGTGCCCTCTAGGTTTAGCGGTAGCGGTAGTGGCACCG |
| | | AGTTCACCCTGACTATCTCTAGCCTGCAGCCCGACGACTTCG |
| | | CTACCTACTACTGTCAGCAGTACTATAACCTGCCCTGGACCT |
| | | TCGGTCAAGGCACTAAGGTCGAGATTAAGCGTACGGTGGCC |
| | | GCTCCCAGCGTGTTCATCTTCCCCCCCAGCGACGAGCAGCTG |
| | | AAGAGCGGCACCGCCAGCGTGGTGTGCCTGCTGAACAACTT |
| | | CTACCCCCGGGAGGCCAAGGTGCAGTGGAAGGTGGACAACG |
| | | CCCTGCAGAGCGGCAACAGCCAGGAGAGCGTCACCGAGCA |
| | | GGACAGCAAGGACTCCACCTACAGCCTGAGCAGCACCCTGA |
| | | CCCTGAGCAAGGCCGACTACGAGAAGCATAAGGTGTACGCC |
| | | TGCGAGGTGACCCACCAGGGCCTGTCCAGCCCCGTGACCAA |
| | | GAGCTTCAACAGGGGCGAGTGC |
| SEQ ID NO: 3116 | DNA | GACATCCAGATGACCCAGTCCCCCTCCAGCCTGTCTGCTTCC |
| | light | GTGGGCGACAGAGTGACCATCACCTGTTCCTCCAGCCAGGA |
| | chain | CATCTCCAACTACCTGAACTGGTATCTGCAGAAGCCCGGCC |
| | | AGTCCCCTCAGCTGCTGATCTACTACACCTCCACCCTGCACC |
| | | TGGGCGTGCCCTCCAGATTTTCCGGCTCTGGCTCTGGCACCG |
| | | AGTTTACCCTGACCATCAGCTCCCTGCAGCCCGACGACTTCG |
| | | CCACCTACTACTGCCAGCAGTACTACAACCTGCCCTGGACCT |
| | | TCGGCCAGGGCACCAAGGTGGAAATCAAGCGTACGGTGGCC |
| | | GCTCCCAGCGTGTTCATCTTCCCCCCAAGCGACGAGCAGCTG |
| | | AAGAGCGGCACCGCCAGCGTGGTGTGTCTGCTGAACAACTT |
| | | CTACCCCAGGGAGGCCAAGGTGCAGTGGAAGGTGGACAAC |
| | | GCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCACAAGGTGTACGC |
| | | CTGTGAGGTGACCCACCAGGGCCTGTCCAGCCCCGTGACCA |
| | | AGAGCTTCAACAGGGGCGAGTGC |
| BAP050-Clone J | | |
| HC | | |
| SEQ ID NO: 3094 | HCDR1 | NYGMN |
| (Kabat) | | |
| SEQ ID NO: 3095 | HCDR2 | WINTDTGEPTYADDFKG |
| (Kabat) | | |
| SEQ ID NO: 3096 | HCDR3 | NPPYYYGTNNAEAMDY |
| (Kabat) | | |
| SEQ ID NO: 3097 | HCDR1 | GFTLTNY |
| (Chothia) | | |
| SEQ ID NO: | HCDR2 | NTDTGE |
| 3098(Chothia) | | |
| SEQ ID NO: | HCDR3 | NPPYYYGTNNAEAMDY |
| 3096(Chothia) | | |
| SEQ ID NO: 3117 | VH | QVQLVQSGAEVKKPGASVKVSCKASGFTLTNYGMNWVRQAP |
| | | GQGLEWMGWINTDTGEPTYADDFKGRFVFSLDTSVSTAYLQIS |
| | | SLKAEDTAVYYCARNPPYYYGTNNAEAMDYWGQGTTVTVSS |
| SEQ ID NO: 3118 | DNA | CAGGTGCAGCTGGTGCAGTCAGGCGCCGAAGTGAAGAAACC |
| | VH | CGGCGCTAGTGTGAAAGTCAGCTGTAAAGCTAGTGGCTTCA |
| | | CCCTGACTAACTACGGGATGAACTGGGTCCGCCAGGCCCCA |
| | | GGTCAAGGCCTCGAGTGGATGGGCTGGATTAACACCGACAC |
| | | CGGCGAGCCTACCTACGCCGACGACTTTAAGGGCAGATTCG |
| | | TGTTTAGCCTGGACACTAGTGTGTCTACCGCCTACCTGCAGA |
| | | TCTCTAGCCTGAAGGCCGAGGACACCGCCGTCTACTACTGC |
| | | GCTAGAAACCCCCCCTACTACTACGGCACTAACAACGCCGA |
| | | GGCTATGGACTACTGGGGTCAAGGCACTACCGTGACCGTGT |
| | | CTAGC |
| SEQ ID NO: 3119 | DNA | CAGGTGCAGCTGGTGCAGTCTGGCGCCGAAGTGAAGAAACC |
| | VH | TGGCGCCTCCGTGAAGGTGTCCTGCAAGGCCTCTGGCTTCAC |
| | | CCTGACCAACTACGGCATGAACTGGGTGCGACAGGCCCCTG |
| | | GACAGGGCCTGGAATGGATGGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCT |
| SEQ ID NO: 3120 | Heavy | QVQLVQSGAEVKKPGASVKVSCKASGFTLTNYGMNWVRQAP |
| | chain | GQGLEWMGWINTDTGEPTYADDFKGRFVFSLDTSVSTAYLQIS |
| | | SLKAEDTAVYYCARNPPYYYGTNNAEAMDYWGQGTTVTVSS |
| | | ASTKGPSVFPLAPCSRSTSESTAALGCLVKDYFPEPVTVSWNSG |
| | | ALTSGVHTFPAVLQSSGLYSLSSVVTVPSSSLGTKTYTCNVDHK |
| | | PSNTKVDKRVESKYGPPCPPCPAPEFLGGPSVFLFPPKPKDTLMI |
| | | SRTPEVTCVVVDVSQEDPEVQFNWYVDGVEVHNAKTKPREEQ |
| | | FNSTYRVVSVLTVLHQDWLNGKEYKCKVSNKGLPSSIEKTISK |
| | | AKGQPREPQVYTLPPSQEEMTKNQVSLTCLVKGFYPSDIAVEW |
| | | ESNGQPENNYKTTPPVLDSDGSFFLYSRLTVDKSRWQEGNVFS |
| | | CSVMHEALHNHYTQKSLSLSLG |
| SEQ ID NO: 3121 | DNA | CAGGTGCAGCTGGTGCAGTCAGGCGCCGAAGTGAAGAAACC |
| | heavy | CGGCGCTAGTGTGAAAGTCAGCTGTAAAGCTAGTGGCTTCA |
| | chain | CCCTGACTAACTACGGGATGAACTGGGTCCGCCAGGCCCCA |
| | | GGTCAAGGCCTCGAGTGGATGGGCTGGATTAACACCGACAC |
| | | CGGCGAGCCTACCTACGCCGACGACTTTAAGGGCAGATTCG |
| | | TGTTTAGCCTGGACACTAGTGTGTCTACCGCCTACCTGCAGA |
| | | TCTCTAGCCTGAAGGCCGAGGACACCGCCGTCTACTACTGC |
| | | GCTAGAAACCCCCCCTACTACTACGGCACTAACAACGCCGA |
| | | GGCTATGGACTACTGGGGTCAAGGCACTACCGTGACCGTGT |
| | | CTAGCGCTAGCACTAAGGGCCCGTCCGTGTTCCCCCTGGCAC |
| | | CTTGTAGCCGGAGCACTAGCGAATCCACCGCTGCCCTCGGCT |
| | | GCCTGGTCAAGGATTACTTCCCGGAGCCCGTGACCGTGTCCT |
| | | GGAACAGCGGAGCCCTGACCTCCGGAGTGCACACCTTCCCC |
| | | GCTGTGCTGCAGAGCTCCGGGCTGTACTCGCTGTCGTCGGTG |
| | | GTCACGGTGCCTTCATCTAGCCTGGGTACCAAGACCTACACT |
| | | TGCAACGTGGACCACAAGCCTTCCAACACTAAGGTGGACAA |
| | | GCGCGTCGAATCGAAGTACGGCCCACCGTGCCCGCCTTGTC |
| | | CCGCGCCGGAGTTCCTCGGCGGTCCCTCGGTCTTTCTGTTCC |
| | | CACCGAAGCCCAAGGACACTTTGATGATTTCCCGCACCCCTG |
| | | AAGTGACATGCGTGGTCGTGGACGTGTCACAGGAAGATCCG |
| | | GAGGTGCAGTTCAATTGGTACGTGGATGGCGTCGAGGTGCA |
| | | CAACGCCAAAACCAAGCCGAGGGAGGAGCAGTTCAACTCCA |
| | | CTTACCGCGTCGTGTCCGTGCTGACGGTGCTGCATCAGGACT |
| | | GGCTGAACGGGAAGGAGTACAAGTGCAAAGTGTCCAACAA |
| | | GGGACTTCCTAGCTCAATCGAAAAGACCATCTCGAAAGCCA |
| | | AGGGACAGCCCCGGGAACCCCAAGTGTATACCCTGCCACCG |
| | | AGCCAGGAAGAAATGACTAAGAACCAAGTCTCATTGACTTG |
| | | CCTTGTGAAGGGCTTCTACCCATCGGATATCGCCGTGGAATG |
| | | GGAGTCCAACGGCCAGCCGGAAAACAACTACAAGACCACCC |
| | | CTCCGGTGCTGGACTCAGACGGATCCTTCTTCCTCTACTCGC |
| | | GGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 3122 | DNA | CAGGTGCAGCTGGTGCAGTCTGGCGCCGAAGTGAAGAAACC |
| | heavy | TGGCGCCTCCGTGAAGGTGTCCTGCAAGGCCTCTGGCTTCAC |
| | chain | CCTGACCAACTACGGCATGAACTGGGTGCGACAGGCCCCTG |
| | | GACAGGGCCTGGAATGGATGGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCTGCTTCTACCAAGGGGCCCAGCGTGTTCCCCCTGGCCCC |
| | | CTGCTCCAGAAGCACCAGCGAGAGCACAGCCGCCCTGGGCT |
| | | GCCTGGTGAAGGACTACTTCCCCGAGCCCGTGACCGTGTCCT |
| | | GGAACAGCGGAGCCCTGACCAGCGGCGTGCACACCTTCCCC |
| | | GCCGTGCTGCAGAGCAGCGGCCTGTACAGCCTGAGCAGCGT |
| | | GGTGACCGTGCCCAGCAGCAGCCTGGGCACCAAGACCTACA |
| | | CCTGTAACGTGGACCACAAGCCCAGCAACACCAAGGTGGAC |
| | | AAGAGGGTGGAGAGCAAGTACGGCCCACCCTGCCCCCCCTG |
| | | CCCAGCCCCCGAGTTCCTGGGCGGACCCAGCGTGTTCCTGTT |
| | | CCCCCCCAAGCCCAAGGACACCCTGATGATCAGCAGAACCC |
| | | CCGAGGTGACCTGTGTGGTGGTGGACGTGTCCCAGGAGGAC |
| | | CCCGAGGTCCAGTTCAACTGGTACGTGGACGGCGTGGAGGT |
| | | GCACAACGCCAAGACCAAGCCCAGAGAGGAGCAGTTTAAC |
| | | AGCACCTACCGGGTGGTGTCCGTGCTGACCGTGCTGCACCA |
| | | GGACTGGCTGAACGGCAAAGAGTACAAGTGTAAGGTCTCCA |
| | | ACAAGGGCCTGCCAAGCAGCATCGAAAAGACCATCAGCAA |
| | | GGCCAAGGGCCAGCCTAGAGAGCCCCAGGTCTACACCCTGC |
| | | CACCCAGCCAAGAGGAGATGACCAAGAACCAGGTGTCCCTG |
| | | ACCTGTCTGGTGAAGGGCTTCTACCCAAGCGACATCGCCGT |
| | | GGAGTGGGAGAGCAACGGCCAGCCCGAGAACAACTACAAG |
| | | ACCACCCCCCCAGTGCTGGACAGCGACGGCAGCTTCTTCCTG |
| | | TACAGCAGGCTGACCGTGGACAAGTCCAGATGGCAGGAGGG |
| | | CAACGTCTTTAGCTGCTCCGTGATGCACGAGGCCCTGCACAA |
| | | CCACTACACCCAGAAGAGCCTGAGCCTGTCCCTGGGC |
| BAP050-Clone J LC | | |
| SEQ ID NO: | LCDR1 | SSSQDISNYLN |
| 3103(Kabat) | | |
| SEQ ID NO: 3104 | LCDR2 | YTSTLHL |
| (Kabat) | | |
| SEQ ID NO: 3105 | LCDR3 | QQYYNLPWT |
| (Kabat) | | |
| SEQ ID NO: 3106 | LCDR1 | SQDISNY |
| (Chothia) | | |
| SEQ ID NO: 3107 | LCDR2 | YTS |
| (Chothia) | | |
| SEQ ID NO: 3108 | LCDR3 | YYNLPW |
| (Chothia) | | |
| SEQ ID NO: 3123 | VL | DIQMTQSPSSLSASVGDRVTITCSSSQDISNYLNWYQQKPGKAP |
| | | KLLIYYTSTLHLGIPPRFSGSGYGTDFTLTINNIESEDAAYYFCQ |
| | | QYYNLPWTFGQGTKVEIK |
| SEQ ID NO: 3124 | DNA | GATATTCAGATGACTCAGTCACCTAGTAGCCTGAGCGCTAGT |
| | VL | GTGGGCGATAGAGTGACTATCACCTGTAGCTCTAGTCAGGA |
| | | TATCTCTAACTACCTGAACTGGTATCAGCAGAAGCCCGGTA |
| | | AAGCCCCTAAGCTGCTGATCTACTACACTAGCACCCTGCACC |
| | | TGGGAATCCCCCCTAGGTTTAGCGGTAGCGGCTACGGCACC |
| | | GACTTCACCCTGACTATTAACAATATCGAGTCAGAGGACGC |
| | | CGCCTACTACTTCTGTCAGCAGTACTATAACCTGCCCTGGAC |
| | | CTTCGGTCAAGGCACTAAGGTCGAGATTAAG |
| SEQ ID NO: 3125 | DNA | GACATCCAGATGACCCAGTCCCCCTCCAGCCTGTCTGCTTCC |
| | VL | GTGGGCGACAGAGTGACCATCACCTGTTCCTCCAGCCAGGA |
| | | CATCTCCAACTACCTGAACTGGTATCAGCAGAAGCCCGGCA |
| | | AGGCCCCCAAGCTGCTGATCTACTACACCTCCACCCTGCACC |
| | | TGGGCATCCCCCCTAGATTCTCCGGCTCTGGCTACGGCACCG |
| | | ACTTCACCCTGACCATCAACAACATCGAGTCCGAGGACGCC |
| | | GCCTACTACTTCTGCCAGCAGTACTACAACCTGCCCTGGACC |
| | | TTCGGCCAGGGCACCAAGGTGGAAATCAAG |
| SEQ ID NO: 3126 | Light | DIQMTQSPSSLSASVGDRVTITCSSSQDISNYLNWYQQKPGKAP |
| | chain | KLLIYYTSTLHLGIPPRFSGSGYGTDFTLTINNIESEDAAYYFCQ |
| | | QYYNLPWTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 3127 | DNA | GATATTCAGATGACTCAGTCACCTAGTAGCCTGAGCGCTAGT |
| | light | GTGGGCGATAGAGTGACTATCACCTGTAGCTCTAGTCAGGA |
| | chain | TATCTCTAACTACCTGAACTGGTATCAGCAGAAGCCCGGTA |
| | | AAGCCCCTAAGCTGCTGATCTACTACACTAGCACCCTGCACC |
| | | TGGGAATCCCCCCTAGGTTTAGCGGTAGCGGCTACGGCACC |
| | | GACTTCACCCTGACTATTAACAATATCGAGTCAGAGGACGC |
| | | CGCCTACTACTTCTGTCAGCAGTACTATAACCTGCCCTGGAC |
| | | CTTCGGTCAAGGCACTAAGGTCGAGATTAAGCGTACGGTGG |
| | | CCGCTCCCAGCGTGTTCATCTTCCCCCCCAGCGACGAGCAGC |
| | | TGAAGAGCGGCACCGCCAGCGTGGTGTGCCTGCTGAACAAC |
| | | TTCTACCCCCGGGAGGCCAAGGTGCAGTGGAAGGTGGACAA |
| | | CGCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCATAAGGTGTACGC |
| | | CTGCGAGGTGACCCACCAGGGCCTGTCCAGCCCCGTGACCA |
| | | AGAGCTTCAACAGGGGCGAGTGC |
| SEQ ID NO: 3128 | DNA | GACATCCAGATGACCCAGTCCCCCTCCAGCCTGTCTGCTTCC |
| | light | GTGGGCGACAGAGTGACCATCACCTGTTCCTCCAGCCAGGA |
| | chain | CATCTCCAACTACCTGAACTGGTATCAGCAGAAGCCCGGCA |
| | | AGGCCCCCAAGCTGCTGATCTACTACACCTCCACCCTGCACC |
| | | TGGGCATCCCCCCTAGATTCTCCGGCTCTGGCTACGGCACCG |
| | | ACTTCACCCTGACCATCAACAACATCGAGTCCGAGGACGCC |
| | | GCCTACTACTTCTGCCAGCAGTACTACAACCTGCCCTGGACC |
| | | TTCGGCCAGGGCACCAAGGTGGAAATCAAGCGTACGGTGGC |
| | | CGCTCCCAGCGTGTTCATCTTCCCCCCAAGCGACGAGCAGCT |
| | | GAAGAGCGGCACCGCCAGCGTGGTGTGTCTGCTGAACAACT |
| | | TCTACCCCAGGGAGGCCAAGGTGCAGTGGAAGGTGGACAAC |
| | | GCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCACAAGGTGTACGC |
| | | CTGTGAGGTGACCCACCAGGGCCTGTCCAGCCCCGTGACCA |
| | | AGAGCTTCAACAGGGGCGAGTGC |
| BAP050-Clone I HC | | |
| SEQ ID NO: 3129 | HCDR1 | AATTACGGGATGAAC |
| (Kabat) | | |
| SEQ ID NO: | HCDR1 | AACTACGGCATGAAC |
| 3130(Kabat) | | |
| SEQ ID NO: 3131 | HCDR2 | TGGATTAACACCGACACCGGGGAGCCTACCTACGCGGACGA |
| (Kabat) | | TTTCAAGGGA |
| SEQ ID NO: | HCDR2 | TGGATCAACACCGACACCGGCGAGCCTACCTACGCCGACGA |
| 3132(Kabat) | | CTTCAAGGGC |
| SEQ ID NO: 3133 | HCDR3 | AACCCGCCCTACTACTACGGAACCAACAACGCCGAAGCCAT |
| (Kabat) | | GGACTAC |
| SEQ ID NO: 3134 | HCDR3 | AACCCCCCTTACTACTACGGCACCAACAACGCCGAGGCCAT |
| (Kabat) | | GGACTAT |
| SEQ ID NO: | HCDR1 | GGATTCACCCTCACCAATTAC |
| 3135(Chothia) | | |
| SEQ ID NO: 3136 | HCDR1 | GGCTTCACCCTGACCAACTAC |
| (Chothia) | | |
| SEQ ID NO: 3137 | HCDR2 | AACACCGACACCGGGGAG |
| (Chothia) | | |
| SEQ ID NO: 3138 | HCDR2 | AACACCGACACCGGCGAG |
| (Chothia) | | |
| SEQ ID NO: 3133 | HCDR3 | AACCCGCCCTACTACTACGGAACCAACAACGCCGAAGCCAT |
| (Chothia) | | GGACTAC |
| SEQ ID NO: | HCDR3 | AACCCCCCTTACTACTACGGCACCAACAACGCCGAGGCCAT |
| 3134(Chothia) | | GGACTAT |
| BAP050-Clone I LC | | |
| SEQ ID NO: | LCDR1 | AGCTCTAGTCAGGATATCTCTAACTACCTGAAC |
| 3139(Kabat) | | |
| SEQ ID NO: 3140 | LCDR1 | TCCTCCAGCCAGGACATCTCCAACTACCTGAAC |
| (Kabat) | | |
| SEQ ID NO: | LCDR2 | TACACTAGCACCCTGCACCTG |
| 3141(Kabat) | | |
| SEQ ID NO: 3142 | LCDR2 | TACACCTCCACCCTGCACCTG |
| (Kabat) | | |
| SEQ ID NO: | LCDR3 | CAGCAGTACTATAACCTGCCCTGGACC |
| 3143(Kabat) | | |
| SEQ ID NO: 3144 | LCDR3 | CAGCAGTACTACAACCTGCCCTGGACC |
| (Kabat) | | |
| SEQ ID NO: 3145 | LCDR1 | AGTCAGGATATCTCTAACTAC |
| (Chothia) | | |
| SEQ ID NO: 3146 | LCDR1 | AGCCAGGACATCTCCAACTAC |
| (Chothia) | | |
| SEQ ID NO: 3147 | LCDR2 | TACACTAGC |
| (Chothia) | | |
| SEQ ID NO: | LCDR2 | TACACCTCC |
| 3148(Chothia) | | |
| SEQ ID NO: 3149 | LCDR3 | TACTATAACCTGCCCTGG |
| (Chothia) | | |
| SEQ ID NO: 164 | LCDR3 | TACTACAACCTGCCCTGG |
| (Chothia) | | |
| BAP050-Clone J | | |
| HC | | |
| SEQ ID NO: | HCDR1 | AACTACGGGATGAAC |
| 3150(Kabat) | | |
| SEQ ID NO: 3130 | HCDR1 | AACTACGGCATGAAC |
| (Kabat) | | |
| SEQ ID NO: 3151 | HCDR2 | TGGATTAACACCGACACCGGCGAGCCTACCTACGCCGACGA |
| (Kabat) | | CTTTAAGGGC |
| SEQ ID NO: 3132 | HCDR2 | TGGATCAACACCGACACCGGCGAGCCTACCTACGCCGACGA |
| (Kabat) | | CTTCAAGGGC |
| SEQ ID NO: 3152 | HCDR3 | AACCCCCCCTACTACTACGGCACTAACAACGCCGAGGCTAT |
| (Kabat) | | GGACTAC |
| SEQ ID NO: 3134 | HCDR3 | AACCCCCCTTACTACTACGGCACCAACAACGCCGAGGCCAT |
| (Kabat) | | GGACTAT |
| SEQ ID NO: 3153 | HCDR1 | GGCTTCACCCTGACTAACTAC |
| (Chothia) | | |
| SEQ ID NO: 3136 | HCDR1 | GGCTTCACCCTGACCAACTAC |
| (Chothia) | | |
| SEQ ID NO: 3137 | HCDR2 | AACACCGACACCGGGGAG |
| (Chothia) | | |
| SEQ ID NO: 3138 | HCDR2 | AACACCGACACCGGCGAG |
| (Chothia) | | |
| SEQ ID NO: 3152 | HCDR3 | AACCCCCCCTACTACTACGGCACTAACAACGCCGAGGCTAT |
| (Chothia) | | GGACTAC |
| SEQ ID NO: 3134 | HCDR3 | AACCCCCCTTACTACTACGGCACCAACAACGCCGAGGCCAT |
| (Chothia) | | GGACTAT |
| BAP050-Clone J LC | | |
| SEQ ID NO: 3139 | LCDR1 | AGCTCTAGTCAGGATATCTCTAACTACCTGAAC |
| (Kabat) | | |
| SEQ ID NO: 3140 | LCDR1 | TCCTCCAGCCAGGACATCTCCAACTACCTGAAC |
| (Kabat) | | |
| SEQ ID NO: 3141 | LCDR2 | TACACTAGCACCCTGCACCTG |
| (Kabat) | | |
| SEQ ID NO: | LCDR2 | TACACCTCCACCCTGCACCTG |
| 3142(Kabat) | | |
| SEQ ID NO: 3143 | LCDR3 | CAGCAGTACTATAACCTGCCCTGGACC |
| (Kabat) | | |
| SEQ ID NO: 3144 | LCDR3 | CAGCAGTACTACAACCTGCCCTGGACC |
| (Kabat) | | |
| SEQ ID NO: 3145 | LCDR1 | AGTCAGGATATCTCTAACTAC |
| (Chothia) | | |
| SEQ ID NO: 3146 | LCDR1 | AGCCAGGACATCTCCAACTAC |
| (Chothia) | | |
| SEQ ID NO: 3147 | LCDR2 | TACACTAGC |
| (Chothia) | | |
| SEQ ID NO: 3148 | LCDR2 | TACACCTCC |
| (Chothia) | | |
| SEQ ID NO: 3149 | LCDR3 | TACTATAACCTGCCCTGG |
| (Chothia) | | |
| SEQ ID NO: 164 | LCDR3 | TACTACAACCTGCCCTGG |
| (Chothia) | | |

Other Exemplary LAG-3 Inhibitors

In one embodiment, the LAG-3 inhibitor is an anti-LAG-3 antibody molecule. In one embodiment, the LAG-3 inhibitor is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 19.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 19. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 19
Amino acid sequences of other exemplary anti-LAG-3 antibody molecules
BMS-986016

| | | |
|---|---|---|
| SEQ ID NO: | Heavy | QVQLQQWGAGLLKPSETLSLTCAVYGGSFSDYYWNWIRQPPGKG |
| 3154 | chain | LEWIGEINHRGSTNSNPSLKSRVTLSLDTSKNQFSLKLRSVTAADTA |
| | | VYYCAFGYSDYEYNWFDPWGQGTLVTVSSASTKGPSVFPLAPCSR |
| | | STSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: | Light | EIVLTQSPATLSLSPGERATLSCRASQSISSYLAWYQQKPGQAPRLLI |
| 3155 | chain | YDASNRATGIPARFSGSGSGTDFTLTISSLEPEDFAVYYCQQRSNWP |
| | | LTFGQGTNLEIKRTVAAPSVFIFPPSDEQLKSGTASVVCLLNNFYPR |
| | | EAKVQWKVDNALQSGNSQESVTEQDSKDSTYSLSSTLTLSKADYE |
| | | KHKVYACEVTHQGLSSPVTKSFNRGEC |

IMP731

| | | |
|---|---|---|
| SEQ ID NO: | Heavy | QVQLKESGPGLVAPSQSLSITCTVSGFSLTAYGVNWVRQPPGKGLE |
| 3156 | chain | WLGMIWDDGSTDYNSALKSRLSISKDNSKSQVFLKMNSLQTDDTA |
| | | RYYCAREGDVAFDYWGQGTTLTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEY |
| | | KCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| SEQ ID NO: | Light | DIVMTQSPSSLAVSVGQKVTMSCKSSQSLLNGSNQKNYLAWYQQ |
| 3157 | chain | KPGQSPKLLVYFASTRDSGVPDRFIGSGSGTDFTLTISSVQAEDLAD |
| | | YFCLQHFGTPPTFGGGTKLEIKRTVAAPSVFIFPPSDEQLKSGTASV |
| | | VCLLNNFYPREAKVQWKVDNALQSGNSQESVTEQDSKDSTYSLSS |
| | | TLTLSKADYEKHKVYACEVTHQGLSSPVTKSFNRGEC |

### TIM-3 Inhibitors

In certain embodiments, the inhibitor of an immune checkpoint molecule is an inhibitor of TIM-3. In some embodiments, the ZBTB32 inhibitor, or a pharmaceutically acceptable salt, hydrate, solvate, prodrug, stereoisomer, or tautomer thereof, of the present disclosure are used in combination with a TIM-3 inhibitor to treat a disease, e.g., cancer. In some embodiments, the TIM-3 inhibitor is MGB453 (Novartis), LY3321367 (Eli Lilly), Sym023 (Symphogen), BGB-A425 (Beigene), INCAGN-2390 (Agenus/Incyte), MBS-986258 (BMS/Five Prime), RO-7121661 (Roche), LY-3415244 (Eli Lilly), or TSR-022 (Tesaro).

Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 20 (e.g., from the heavy and light chain variable region sequences of ABTIM3-hum11 or ABTIM3-hum03 disclosed in Table 20), or encoded by a nucleotide sequence shown in Table 20. In some embodiments, the CDRs are according to the Kabat definition (e.g., as set out in Table 20). In some embodiments, the CDRs are according to the Chothia definition (e.g., as set out in Table 20). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 20, or encoded by a nucleotide sequence shown in Table 20.

In one embodiment, the anti-TIM-3 antibody molecule comprises a heavy chain variable region (VH) comprising a VHCDR1 amino acid sequence of SEQ ID NO: 3159, a VHCDR2 amino acid sequence of SEQ ID NO: 3160, and a VHCDR3 amino acid sequence of SEQ ID NO: 3161; and a light chain variable region (VL) comprising a VLCDR1 amino acid sequence of SEQ ID NO: 3168, a VLCDR2 amino acid sequence of SEQ ID NO: 3169, and a VLCDR3 amino acid sequence of SEQ ID NO: 3170, each disclosed in Table 20. In one embodiment, the anti-TIM-3 antibody molecule comprises a heavy chain variable region (VH) comprising a VHCDR1 amino acid sequence of SEQ ID NO: 3159, a VHCDR2 amino acid sequence of SEQ ID NO: 3178, and a VHCDR3 amino acid sequence of SEQ ID NO: 3161; and a light chain variable region (VL) comprising a VLCDR1 amino acid sequence of SEQ ID NO: 3168, a VLCDR2 amino acid sequence of SEQ ID NO: 3169, and a VLCDR3 amino acid sequence of SEQ ID NO: 3170, each disclosed in Table 20.

In one embodiment, the anti-TIM-3 antibody molecule comprises a VH comprising the amino acid sequence of SEQ ID NO: 3164, or an amino acid sequence at least 85%, 90%, 95%, or 99% identical or higher to SEQ ID NO: 3164. In one embodiment, the anti-TIM-3 antibody molecule comprises a VL comprising the amino acid sequence of SEQ ID NO: 3174, or an amino acid sequence at least 85%, 90%, 95%, or 99% identical or higher to SEQ ID NO: 3174. In one embodiment, the anti-TIM-3 antibody molecule comprises a VH comprising the amino acid sequence of SEQ ID NO: 3180, or an amino acid sequence at least 85%, 90%, 95%, or 99% identical or higher to SEQ ID NO: 3180.

In one embodiment, the anti-TIM-3 antibody molecule comprises a VL comprising the amino acid sequence of SEQ ID NO: 3184, or an amino acid sequence at least 85%, 90%, 95%, or 99% identical or higher to SEQ ID NO: 3184. In one embodiment, the anti-TIM-3 antibody molecule comprises a VH comprising the amino acid sequence of SEQ ID NO: 3164 and a VL comprising the amino acid sequence of SEQ ID NO: 3174. In one embodiment, the anti-TIM-3 antibody molecule comprises a VH comprising the amino acid sequence of SEQ ID NO: 3180 and a VL comprising the amino acid sequence of SEQ ID NO: 3184.

In one embodiment, the antibody molecule comprises a VH encoded by the nucleotide sequence of SEQ ID NO: 3165, or a nucleotide sequence at least 85%, 90%, 95%, or 99% identical or higher to SEQ ID NO: 3165. In one embodiment, the antibody molecule comprises a VL encoded by the nucleotide sequence of SEQ ID NO: 3175, or a nucleotide sequence at least 85%, 90%, 95%, or 99% identical or higher to SEQ ID NO: 3175. In one embodiment, the antibody molecule comprises a VH encoded by the nucleotide sequence of SEQ ID NO: 3181, or a nucleotide sequence at least 85%, 90%, 95%, or 99% identical or higher to SEQ ID NO: 3181. In one embodiment, the antibody molecule comprises a VL encoded by the nucleotide sequence of SEQ ID NO: 3185, or a nucleotide sequence at least 85%, 90%, 95%, or 99% identical or higher to SEQ ID NO: 3185. In one embodiment, the antibody molecule comprises a VH encoded by the nucleotide sequence of SEQ ID NO: 3165 and a VL encoded by the nucleotide sequence of SEQ ID NO: 3175. In one embodiment, the antibody molecule comprises a VH encoded by the nucleotide sequence of SEQ ID NO: 3181 and a VL encoded by the nucleotide sequence of SEQ ID NO: 3185.

In one embodiment, the anti-TIM-3 antibody molecule comprises a heavy chain comprising the amino acid sequence of SEQ ID NO: 3166, or an amino acid sequence at least 85%, 90%, 95%, or 99% identical or higher to SEQ ID NO: 3166. In one embodiment, the anti-TIM-3 antibody molecule comprises a light chain comprising the amino acid sequence of SEQ ID NO: 3176, or an amino acid sequence at least 85%, 90%, 95%, or 99% identical or higher to SEQ ID NO: 3176. In one embodiment, the anti-TIM-3 antibody molecule comprises a heavy chain comprising the amino acid sequence of SEQ ID NO: 3182, or an amino acid sequence at least 85%, 90%, 95%, or 99% identical or higher to SEQ ID NO: 3182. In one embodiment, the anti-TIM-3 antibody molecule comprises a light chain comprising the amino acid sequence of SEQ ID NO: 3186, or an amino acid sequence at least 85%, 90%, 95%, or 99% identical or higher to SEQ ID NO: 3186. In one embodiment, the anti-TIM-3 antibody molecule comprises a heavy chain comprising the amino acid sequence of SEQ ID NO: 3166 and a light chain comprising the amino acid sequence of SEQ ID NO: 3176. In one embodiment, the anti-TIM-3 antibody molecule comprises a heavy chain comprising the amino acid sequence of SEQ ID NO: 3182 and a light chain comprising the amino acid sequence of SEQ ID NO: 3186.

In one embodiment, the antibody molecule comprises a heavy chain encoded by the nucleotide sequence of SEQ ID NO: 3167, or a nucleotide sequence at least 85%, 90%, 95%, or 99% identical or higher to SEQ ID NO: 3167. In one embodiment, the antibody molecule comprises a light chain encoded by the nucleotide sequence of SEQ ID NO: 3177, or a nucleotide sequence at least 85%, 90%, 95%, or 99% identical or higher to SEQ ID NO: 3177. In one embodiment, the antibody molecule comprises a heavy chain encoded by the nucleotide sequence of SEQ ID NO: 3183, or a nucleotide sequence at least 85%, 90%, 95%, or 99% identical or higher to SEQ ID NO: 3183. In one embodiment, the antibody molecule comprises a light chain encoded by the nucleotide sequence of SEQ ID NO: 3187, or a nucleotide sequence at least 85%, 90%, 95%, or 99% identical or higher to SEQ ID NO: 3187. In one embodiment, the antibody molecule comprises a heavy chain encoded by the nucleotide sequence of SEQ ID NO: 3167 and a light chain encoded by the nucleotide sequence of SEQ ID NO: 3177. In one embodiment, the antibody molecule comprises a heavy chain encoded by the nucleotide sequence of SEQ ID NO: 3183 and a light chain encoded by the nucleotide sequence of SEQ ID NO: 3187.

The antibody molecules described herein can be made by vectors, host cells, and methods described in US 2015/0218274, incorporated by reference in its entirety.

TABLE 20
Amino acid and nucleotide sequences of exemplary anti-TIM-3 antibody molecules
ABTIM3-hum11

| | | |
|---|---|---|
| SEQ ID NO: 3159 | HCDR1 | SYNMH |
| (Kabat) | | |
| SEQ ID NO: 3160 | HCDR2 | DIYPGNGDTSYNQKFKG |
| (Kabat) | | |
| SEQ ID NO: 3161 | HCDR3 | VGGAFPMDY |
| (Kabat) | | |
| SEQ ID NO: | HCDR1 | GYTFTSY |
| 3162(Chothia) | | |
| SEQ ID NO: | HCDR2 | YPGNGD |
| 3163(Chothia) | | |
| SEQ ID NO: 3161 | HCDR3 | VGGAFPMDY |
| (Chothia) | | |
| SEQ ID NO: 3164 | VH | QVQLVQSGAEVKKPGSSVKVSCKASGYTFTSYNMHWVRQAPGQ |
| | | GLEWMGDIYPGNGDTSYNQKFKGRVTITADKSTSTVYMELSSLR |
| | | SEDTAVYYCARVGGAFPMDYWGQGTTVTVSS |
| SEQ ID NO: 3165 | DNA | CAGGTGCAGCTGGTGCAGTCAGGCGCCGAAGTGAAGAAACCC |
| | VH | GGCTCTAGCGTGAAAGTTTCTTGTAAAGCTAGTGGCTACACCT |
| | | TCACTAGCTATAATATGCACTGGGTTCGCCAGGCCCCAGGGCA |
| | | AGGCCTCGAGTGGATGGGCGATATCTACCCCGGGAACGGCGA |
| | | CACTAGTTATAATCAGAAGTTTAAGGGTAGAGTCACTATCACC |
| | | GCCGATAAGTCTACTAGCACCGTCTATATGGAACTGAGTTCCC |
| | | TGAGGTCTGAGGACACCGCCGTCTACTACTGCGCTAGAGTGG |
| | | GCGGAGCCTTCCCTATGGACTACTGGGGTCAAGGCACTACCGT |
| | | GACCGTGTCTAGC |
| SEQ ID NO: 3166 | Heavy | QVQLVQSGAEVKKPGSSVKVSCKASGYTFTSYNMHWVRQAPGQ |
| | chain | GLEWMGDIYPGNGDTSYNQKFKGRVTITADKSTSTVYMELSSLR |
| | | SEDTAVYYCARVGGAFPMDYWGQGTTVTVSSASTKGPSVFPLA |
| | | PCSRSTSESTAALGCLVKDYFPEPVTVSWNSGALTSGVHTFPAVL |
| | | QSSGLYSLSSVVTVPSSSLGTKTYTCNVDHKPSNTKVDKRVESKY |
| | | GPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQ |
| | | DWLNGKEYKCKVSNKGLPSSIEKTISKAKGQPREPQVYTLPPSQE |
| | | EMTKNQVSLTCLVKGFYPSDIAVEWESNGQPENNYKTTPPVLDS |
| | | DGSFFLYSRLTVDKSRWQEGNVFSCSVMHEALHNHYTQKSLSLS |
| | | LG |
| SEQ ID NO: 3167 | DNA | CAGGTGCAGCTGGTGCAGTCAGGCGCCGAAGTGAAGAAACCC |
| | heavy | GGCTCTAGCGTGAAAGTTTCTTGTAAAGCTAGTGGCTACACCT |
| | chain | TCACTAGCTATAATATGCACTGGGTTCGCCAGGCCCCAGGGCA |
| | | AGGCCTCGAGTGGATGGGCGATATCTACCCCGGGAACGGCGA |
| | | CACTAGTTATAATCAGAAGTTTAAGGGTAGAGTCACTATCACC |
| | | GCCGATAAGTCTACTAGCACCGTCTATATGGAACTGAGTTCCC |
| | | TGAGGTCTGAGGACACCGCCGTCTACTACTGCGCTAGAGTGG |
| | | GCGGAGCCTTCCCTATGGACTACTGGGGTCAAGGCACTACCGT |
| | | GACCGTGTCTAGCGCTAGCACTAAGGGCCCGTCCGTGTTCCCC |
| | | CTGGCACCTTGTAGCCGGAGCACTAGCGAATCCACCGCTGCCC |
| | | TCGGCTGCCTGGTCAAGGATTACTTCCCGGAGCCCGTGACCGT |
| | | GTCCTGGAACAGCGGAGCCCTGACCTCCGGAGTGCACACCTTC |
| | | CCCGCTGTGCTGCAGAGCTCCGGGCTGTACTCGCTGTCGTCGG |
| | | TGGTCACGGTGCCTTCATCTAGCCTGGGTACCAAGACCTACAC |
| | | TTGCAACGTGGACCACAAGCCTTCCAACACTAAGGTGGACAA |
| | | GCGCGTCGAATCGAAGTACGGCCCACCGTGCCCGCCTTGTCCC |
| | | GCGCCGGAGTTCCTCGGCGGTCCCTCGGTCTTTCTGTTCCCAC |
| | | CGAAGCCCAAGGACACTTTGATGATTTCCCGCACCCCTGAAGT |
| | | GACATGCGTGGTCGTGGACGTGTCACAGGAAGATCCGGAGGT |
| | | GCAGTTCAATTGGTACGTGGATGGCGTCGAGGTGCACAACGC |
| | | CAAAACCAAGCCGAGGGAGGAGCAGTTCAACTCCACTTACCG |
| | | CGTCGTGTCCGTGCTGACGGTGCTGCATCAGGACTGGCTGAAC |
| | | GGGAAGGAGTACAAGTGCAAAGTGTCCAACAAGGGACTTCCT |
| | | AGCTCAATCGAAAAGACCATCTCGAAAGCCAAGGGACAGCCC |
| | | CGGGAACCCCAAGTGTATACCCTGCCACCGAGCCAGGAAGAA |
| | | ATGACTAAGAACCAAGTCTCATTGACTTGCCTTGTGAAGGGCT |
| | | TCTACCCATCGGATATCGCCGTGGAATGGGAGTCCAACGGCC |
| | | AGCCGGAAAACAACTACAAGACCACCCCTCCGGTGCTGGACT |
| | | CAGACGGATCCTTCTTCCTCTACTCGCGGCTGACCGTGGATAA |
| | | GAGCAGATGGCAGGAGGGAAATGTGTTCAGCTGTTCTGTGAT |
| | | GCATGAAGCCCTGCACAACCACTACACTCAGAAGTCCCTGTCC |
| | | CTCTCCCTGGGA |
| SEQ ID NO: 3168 | LCDR1 | RASESVEYYGTSLMQ |
| (Kabat) | | |
| SEQ ID NO: | LCDR2 | AASNVES |
| 3169(Kabat) | | |
| SEQ ID NO: 3170 | LCDR3 | QQSRKDPST |
| (Kabat) | | |
| SEQ ID NO: 3171 | LCDR1 | SESVEYYGTSL |
| (Chothia) | | |
| SEQ ID NO: 3172 | LCDR2 | AAS |
| (Chothia) | | |
| SEQ ID NO: 3173 | LCDR3 | SRKDPS |
| (Chothia) | | |
| SEQ ID NO: 3174 | VL | AIQLTQSPSSLSASVGDRVTITCRASESVEYYGTSLMQWYQQKPG |
| | | KAPKLLIYAASNVESGVPSRFSGSGSGTDFTLTISSLQPEDFATYF |
| | | CQQSRKDPSTFGGGTKVEIK |
| SEQ ID NO: 3175 | DNA | GCTATTCAGCTGACTCAGTCACCTAGTAGCCTGAGCGCTAGTG |
| | VL | TGGGCGATAGAGTGACTATCACCTGTAGAGCTAGTGAATCAG |
| | | TCGAGTACTACGGCACTAGCCTGATGCAGTGGTATCAGCAGA |
| | | AGCCCGGGAAAGCCCCTAAGCTGCTGATCTACGCCGCCTCTAA |
| | | CGTGGAATCAGGCGTGCCCTCTAGGTTTAGCGGTAGCGGTAGT |
| | | GGCACCGACTTCACCCTGACTATCTCTAGCCTGCAGCCCGAGG |
| | | ACTTCGCTACCTACTTCTGTCAGCAGTCTAGGAAGGACCCTAG |
| | | CACCTTCGGCGGAGGCACTAAGGTCGAGATTAAG |
| SEQ ID NO: 3176 | Light | AIQLTQSPSSLSASVGDRVTITCRASESVEYYGTSLMQWYQQKPG |
| | chain | KAPKLLIYAASNVESGVPSRFSGSGSGTDFTLTISSLQPEDFATYF |
| | | CQQSRKDPSTFGGGTKVEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |
| SEQ ID NO: 3177 | DNA | GCTATTCAGCTGACTCAGTCACCTAGTAGCCTGAGCGCTAGTG |
| | light | TGGGCGATAGAGTGACTATCACCTGTAGAGCTAGTGAATCAG |
| | chain | TCGAGTACTACGGCACTAGCCTGATGCAGTGGTATCAGCAGA |
| | | AGCCCGGGAAAGCCCCTAAGCTGCTGATCTACGCCGCCTCTAA |
| | | CGTGGAATCAGGCGTGCCCTCTAGGTTTAGCGGTAGCGGTAGT |
| | | GGCACCGACTTCACCCTGACTATCTCTAGCCTGCAGCCCGAGG |
| | | ACTTCGCTACCTACTTCTGTCAGCAGTCTAGGAAGGACCCTAG |
| | | CACCTTCGGCGGAGGCACTAAGGTCGAGATTAAGCGTACGGT |
| | | GGCCGCTCCCAGCGTGTTCATCTTCCCCCCCAGCGACGAGCAG |
| | | CTGAAGAGCGGCACCGCCAGCGTGGTGTGCCTGCTGAACAAC |
| | | TTCTACCCCCGGGAGGCCAAGGTGCAGTGGAAGGTGGACAAC |
| | | GCCCTGCAGAGCGGCAACAGCCAGGAGAGCGTCACCGAGCAG |
| | | GACAGCAAGGACTCCACCTACAGCCTGAGCAGCACCCTGACC |
| | | CTGAGCAAGGCCGACTACGAGAAGCATAAGGTGTACGCCTGC |
| | | GAGGTGACCCACCAGGGCCTGTCCAGCCCCGTGACCAAGAGC |
| | | TTCAACAGGGGCGAGTGC |

ABTIM3-hum03

| | | |
|---|---|---|
| SEQ ID NO: 3159 | HCDR1 | SYNMH |
| (Kabat) | | |
| SEQ ID NO: 3178 | HCDR2 | DIYPGQGDTSYNQKFKG |
| (Kabat) | | |
| SEQ ID NO: 3161 | HCDR3 | VGGAFPMDY |
| (Kabat) | | |
| SEQ ID NO: 3162 | HCDRI | GYTFTSY |
| (Chothia) | | |
| SEQ ID NO: 3179 | HCDR2 | YPGQGD |
| (Chothia) | | |
| SEQ ID NO: 3161 | HCDR3 | VGGAFPMDY |
| (Chothia) | | |
| SEQ ID NO: 3180 | VH | QVQLVQSGAEVKKPGASVKVSCKASGYTFTSYNMHWVRQAPG |
| | | QGLEWIGDIYPGQGDTSYNQKFKGRATMTADKSTSTVYMELSSL |
| | | RSEDTAVYYCARVGGAFPMDYWGQGTLVTVSS |
| SEQ ID NO: 3181 | DNA | CAGGTGCAGCTGGTGCAGTCAGGCGCCGAAGTGAAGAAACCC |
| | VH | GGCGCTAGTGTGAAAGTTAGCTGTAAAGCTAGTGGCTATACTT |
| | | TCACTTCTTATAATATGCACTGGGTCCGCCAGGCCCCAGGTCA |
| | | AGGCCTCGAGTGGATCGGCGATATCTACCCCGGTCAAGGCGA |
| | | CACTTCCTATAATCAGAAGTTTAAGGGTAGAGCTACTATGACC |
| | | GCCGATAAGTCTACTICTACCGTCTATATGGAACTGAGTTCCC |
| | | TGAGGTCTGAGGACACCGCCGTCTACTACTGCGCTAGAGTGG |
| | | GCGGAGCCTTCCCAATGGACTACTGGGGTCAAGGCACCCTGG |
| | | TCACCGTGTCTAGC |
| SEQ ID NO: 3182 | Heavy | QVQLVQSGAEVKKPGASVKVSCKASGYTFTSYNMHWVRQAPG |
| | chain | QGLEWIGDIYPGQGDTSYNQKFKGRATMTADKSTSTVYMELSSL |
| | | RSEDTAVYYCARVGGAFPMDYWGQGTLVTVSSASTKGPSVFPL |
| | | APCSRSTSESTAALGCLVKDYFPEPVTVSWNSGALTSGVHTFPAV |
| | | LQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLH |
| | | QDWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLSL |
| | | SLG |
| SEQ ID NO: 3183 | DNA | CAGGTGCAGCTGGTGCAGTCAGGCGCCGAAGTGAAGAAACCC |
| | heavy | GGCGCTAGTGTGAAAGTTAGCTGTAAAGCTAGTGGCTATACTT |
| | chain | TCACTTCTTATAATATGCACTGGGTCCGCCAGGCCCCAGGTCA |
| | | AGGCCTCGAGTGGATCGGCGATATCTACCCCGGTCAAGGCGA |
| | | CACTTCCTATAATCAGAAGTTTAAGGGTAGAGCTACTATGACC |
| | | GCCGATAAGTCTACTTCTACCGTCTATATGGAACTGAGTTCCC |
| | | TGAGGTCTGAGGACACCGCCGTCTACTACTGCGCTAGAGTGG |
| | | GCGGAGCCTTCCCAATGGACTACTGGGGTCAAGGCACCCTGG |
| | | TCACCGTGTCTAGCGCTAGCACTAAGGGCCCGTCCGTGTTCCC |
| | | CCTGGCACCTTGTAGCCGGAGCACTAGCGAATCCACCGCTGCC |
| | | CTCGGCTGCCTGGTCAAGGATTACTTCCCGGAGCCCGTGACCG |
| | | TGTCCTGGAACAGCGGAGCCCTGACCTCCGGAGTGCACACCTT |
| | | CCCCGCTGTGCTGCAGAGCTCCGGGCTGTACTCGCTGTCGTCG |
| | | GTGGTCACGGTGCCTTCATCTAGCCTGGGTACCAAGACCTACA |
| | | CTTGCAACGTGGACCACAAGCCTTCCAACACTAAGGTGGACA |
| | | AGCGCGTCGAATCGAAGTACGGCCCACCGTGCCCGCCTTGTCC |
| | | CGCGCCGGAGTTCCTCGGCGGTCCCTCGGTCTTTCTGTTCCCA |
| | | CCGAAGCCCAAGGACACTTTGATGATTTCCCGCACCCCTGAAG |
| | | TGACATGCGTGGTCGTGGACGTGTCACAGGAAGATCCGGAGG |
| | | TGCAGTTCAATTGGTACGTGGATGGCGTCGAGGTGCACAACG |
| | | CCAAAACCAAGCCGAGGGAGGAGCAGTTCAACTCCACTTACC |
| | | GCGTCGTGTCCGTGCTGACGGTGCTGCATCAGGACTGGCTGAA |
| | | CGGGAAGGAGTACAAGTGCAAAGTGTCCAACAAGGGACTTCC |
| | | TAGCTCAATCGAAAAGACCATCTCGAAAGCCAAGGGACAGCC |
| | | CCGGGAACCCCAAGTGTATACCCTGCCACCGAGCCAGGAAGA |
| | | AATGACTAAGAACCAAGTCTCATTGACTTGCCTTGTGAAGGGC |
| | | TTCTACCCATCGGATATCGCCGTGGAATGGGAGTCCAACGGCC |
| | | AGCCGGAAAACAACTACAAGACCACCCCTCCGGTGCTGGACT |
| | | CAGACGGATCCTTCTTCCTCTACTCGCGGCTGACCGTGGATAA |
| | | GAGCAGATGGCAGGAGGGAAATGTGTTCAGCTGTTCTGTGAT |
| | | GCATGAAGCCCTGCACAACCACTACACTCAGAAGTCCCTGTCC |
| | | CTCTCCCTGGGA |
| SEQ ID NO: 3168 | LCDR1 | RASESVEYYGTSLMQ |
| (Kabat) | | |
| SEQ ID NO: 3169 | LCDR2 | AASNVES |
| (Kabat) | | |
| SEQ ID NO: 3170 | LCDR3 | QQSRKDPST |
| (Kabat) | | |
| SEQ ID NO: | LCDR1 | SESVEYYGTSL |
| 3171(Chothia) | | |
| SEQ ID NO: 3172 | LCDR2 | AAS |
| (Chothia) | | |
| SEQ ID NO: 3173 | LCDR3 | SRKDPS |
| (Chothia). | | |
| SEQ ID NO: 3184 | VL | DIVLTQSPDSLAVSLGERATINCRASESVEYYGTSLMQWYQQKP |
| | | GQPPKLLIYAASNVESGVPDRFSGSGSGTDFTLTISSLQAEDVAVY |
| | | YCQQSRKDPSTFGGGTKVEIK |
| SEQ ID NO: 3185 | DNA | GATATCGTCCTGACTCAGTCACCCGATAGCCTGGCCGTCAGCC |
| | VL | TGGGCGAGCGGGCTACTATTAACTGTAGAGCTAGTGAATCAG |
| | | TCGAGTACTACGGCACTAGCCTGATGCAGTGGTATCAGCAGA |
| | | AGCCCGGTCAACCCCCTAAGCTGCTGATCTACGCCGCCTCTAA |
| | | CGTGGAATCAGGCGTGCCCGATAGGTTTAGCGGTAGCGGTAG |
| | | TGGCACCGACTTCACCCTGACTATTAGTAGCCTGCAGGCCGAG |
| | | GACGTGGCCGTCTACTACTGTCAGCAGTCTAGGAAGGACCCTA |
| | | GCACCTTCGGCGGAGGCACTAAGGTCGAGATTAAG |
| SEQ ID NO: 3186 | Light | DIVLTQSPDSLAVSLGERATINCRASESVEYYGTSLMQWYQQKP |
| | chain | GQPPKLLIYAASNVESGVPDRFSGSGSGTDFTLTISSLQAEDVAVY |
| | | YCQQSRKDPSTFGGGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 3187 | DNA | GATATCGTCCTGACTCAGTCACCCGATAGCCTGGCCGTCAGCC |
| | light | TGGGCGAGCGGGCTACTATTAACTGTAGAGCTAGTGAATCAG |
| | chain | TCGAGTACTACGGCACTAGCCTGATGCAGTGGTATCAGCAGA |
| | | AGCCCGGTCAACCCCCTAAGCTGCTGATCTACGCCGCCTCTAA |
| | | CGTGGAATCAGGCGTGCCCGATAGGTTTAGCGGTAGCGGTAG |
| | | TGGCACCGACTTCACCCTGACTATTAGTAGCCTGCAGGCCGAG |
| | | GACGTGGCCGTCTACTACTGTCAGCAGTCTAGGAAGGACCCTA |
| | | GCACCTTCGGCGGAGGCACTAAGGTCGAGATTAAGCGTACGG |
| | | TGGCCGCTCCCAGCGTGTTCATCTTCCCCCCCAGCGACGAGCA |
| | | GCTGAAGAGCGGCACCGCCAGCGTGGTGTGCCTGCTGAACAA |
| | | CTTCTACCCCCGGGAGGCCAAGGTGCAGTGGAAGGTGGACAA |
| | | CGCCCTGCAGAGCGGCAACAGCCAGGAGAGCGTCACCGAGCA |
| | | GGACAGCAAGGACTCCACCTACAGCCTGAGCAGCACCCTGAC |
| | | CCTGAGCAAGGCCGACTACGAGAAGCATAAGGTGTACGCCTG |
| | | CGAGGTGACCCACCAGGGCCTGTCCAGCCCCGTGACCAAGAG |
| | | CTTCAACAGGGGCGAGTGC |

Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 21. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

In one embodiment, the anti-TIM-3 antibody molecule is LY3321367 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of LY3321367.

In one embodiment, the anti-TIM-3 antibody molecule is Sym023 (Symphogen). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of Sym023.

In one embodiment, the anti-TIM-3 antibody molecule is BGB-A425 (Beigene). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BGB-A425.

In one embodiment, the anti-TIM-3 antibody molecule is INCAGN-2390 (Agenus/Incyte). In one embodiment, the anti-TIM-3 antibody molecule comprises one or more of the CDR sequences (or collectively all of the CDR sequences), the heavy chain variable region sequence and/or light chain variable region sequence, or the heavy chain or light chain sequence of INCAGN-2390.

In one embodiment, the anti-TIM-3 antibody molecule is BMS-986258 (BMS/Five Prime). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BMS-986258.

In one embodiment, the anti-TIM-3 antibody or inhibitor molecule is RO-7121661 (Roche). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of the TIM-3 binding arm of RO-7121661.

In one embodiment, the anti-TIM-3 antibody or inhibitor molecule is LY-3415244 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of the TIM-3 binding arm of LY-3415244.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 21
Amino acid sequences of other exemplary anti-TIM-3 antibody molecules
APE5137

| | | |
|---|---|---|
| SEQ ID NO: | VH | EVQLLESGGGLVQPGGSLRLSCAAASGFTFSSYDMSWVRQAPGKGLD |
| 3188 | | WVSTISGGGTYTYYQDSVKGRFTISRDNSKNTLYLQMNSLRAEDTAV |
| | | YYCASMDYWGQGTTVTVSSA |
| SEQ ID NO: | VL | DIQMTQSPSSLSASVGDRVTITCRASQSIRRYLNWYHQKPGKAPKLLI |
| 3189 | | YGASTLQSGVPSRFSGSGSGTDFTLTISSLQPEDFAVYYCQQSHSAPLT |
| | | FGGGTKVEIKR |

APE5121

| | | |
|---|---|---|
| SEQ ID NO: | VH | EVQVLESGGGLVQPGGSLRLYCVASGFTFSGSYAMSWVRQAPGKGL |
| 3190 | | EWVSAISGSGGSTYYADSVKGRFTISRDNSKNTLYLQMNSLRAEDTA |
| | | VYYCAKKYYVGPADYWGQGTLVTVSSG |
| SEQ ID NO: | VL | DIVMTQSPDSLAVSLGERATINCKSSQSVLYSSNNKNYLAWYQHKPG |
| 3191 | | QPPKLLIYWASTRESGVPDRFSGSGSGTDFTLTISSLQAEDVAVYYCQ |
| | | QYYSSPLTFGGGTKIEVK |

### Cytokines

In yet another embodiment, the ZBTB32 inhibitor, or a pharmaceutically acceptable salt, hydrate, solvate, prodrug, stereoisomer, or tautomer thereof, of the present disclosure are used in combination with one or more cytokines, including but not limited to, interferon, IL-2, IL-15, IL-7, or IL21. In certain embodiments, ZBTB32 inhibitor, or a pharmaceutically acceptable salt, hydrate, solvate, prodrug, stereoisomer, or tautomer thereof, are administered in combination with an IL-15/IL-15Ra complex. In some embodiments, the IL-15/IL-15Ra complex is selected from NIZ985 (Novartis), ATL-803 (Altor) or CYP0150 (Cytune).

Exemplary IL-15 IL-15Ra Complexes

In one embodiment, the cytokine is IL-15 complexed with a soluble form of IL-15 receptor alpha (IL-15Ra). The IL-15/IL-15Ra complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the formulation comprises an amino acid sequence of SEQ ID NO: 3192 in Table 22 or an amino acid sequence at least 85%, 90%, 95%, or 99% identical or higher to SEQ ID NO: 3192, and the soluble form of human IL-15Ra comprises an amino acid sequence of SEQ ID NO: 3193 in Table 22, or an amino acid sequence at least 85%, 90%, 95%, or 99% identical or higher to SEQ ID NO: 3193, as described in WO 2014/066527, incorporated by reference in its entirety. The molecules described herein can be made by vectors, host cells, and methods described in WO 2007084342, incorporated by reference in its entirety.

TABLE 22
Exemplary Amino acid and nucleotide sequences of exemplary IL-15/IL-15Ra
complexes

| | | |
|---|---|---|
| NIZ985 | | |
| SEQ ID NO: | Human | NWVNVISDLKKIEDLIQSMHIDATLYTESDVHPSCKVTAMKCFL |
| 3192 | IL-15 | LELQVISLESGDASIHDTVENLIILANNSLSSNGNVTESGCKECEE |
| | | LEEKNIKEFLQSFVHIVQMFINTS |
| SEQ ID NO: | Human | ITCPPPMSVEHADIWVKSYSLYSRERYICNSGFKRKAGTSSLTEC |
| 3193 | Soluble | VLNKATNVAHWTTPSLKCIRDPALVHQRPAPPSTVTTAGVTPQP |
| | IL-15Ra | ESLSPSGKEPAASSPSSNNTAATTAAIVPGSQLMPSKSPSTGTTEI |
| | | SSHESSHGTPSQTTAKNWELTASASHQPPGVYPQG |

Other Exemplary IL-15 IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is described in WO 2008/143794, incorporated by reference in its entirety. In one embodiment, the IL-15/IL-15Ra Fc fusion protein comprises the sequences as disclosed in Table 23.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is described in WO 2007/04606 and WO 2012/175222, incorporated by reference in their entirety. In one embodiment, the IL-15/IL-15Ra sushi domain fusion comprises the sequences as disclosed in Table 23.

TABLE 23
Exemplary Amino acid sequences of other exemplary IL-15/IL-15Ra complexes
ALT-803

| | | |
|---|---|---|
| SEQ ID NO: | IL-15N72D | NWVNVISDLKKIEDLIQSMHIDATLYTESDVHPSCKVTAMKCF |
| 3194 | | LLELQVISLESGDASIHDTVENLIILANDSLSSNGNVTESGCKEC |
| | | EELEEKNIKEFLQSFVHIVQMFINTS |
| SEQ ID NO: | IL-15RaSu/ Fc | ITCPPPMSVEHADIWVKSYSLYSRERYICNSGFKRKAGTSSLTE |
| 3195 | | CVLNKATNVAHWTTPSLKCIREPKSCDKTHTCPPCPAPELLGG |
| | | PSVFLFPPKPKDTLMISRTPEVTCVVVDVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVSL |
| | | TCLVKGFYPSDIAVEWESNGQPENNYKTTPPVLDSDGSFFLYS |
| | | KLTVDKSRWQQGNVFSCSVMHEALHNHYTQKSLSLSPGK |

IL-15 / IL-15Ra sushi domain fusion (CYP0150)

| | | |
|---|---|---|
| SEQ ID NO: | Human IL-15 | NWVNVISDLKKIEDLIQSMHIDATLYTESDVHPSCKVTAMKCF |
| 3196 | | LLELQVISLESGDASIHDTVENLIILANNSLSSNGNVTESGCKEC |
| | | EELEXKNIKEFLQSFVHIVQMFINTS |
| | | Where X is E or K |
| SEQ ID NO: | Human IL- | ITCPPPMSVEHADIWVKSYSLYSRERYICNSGFKRKAGTSSLTE |
| 3197 | 15Ra sushi | CVLNKATNVAHWTTPSLKCIRDPALVHQRPAPP |
| | and hinge | |
| | domains | |

In yet another embodiment, the ZBTB32 inhibitor, or a pharmaceutically acceptable salt, hydrate, solvate, prodrug, stereoisomer, or tautomer thereof, of the present disclosure are used in combination with one or more agonists of toll like receptors (TLRs, e.g., TLR7, TLR8, TLR9) to treat a disease, e.g., cancer. In some embodiments, a compound of the present disclosure can be used in combination with a TLR7 agonist or a TLR7 agonist conjugate.

In some embodiments, the TLR7 agonist comprises a compound disclosed in International Application Publication No. WO2011/049677, which is hereby incorporated by reference in its entirety. In some embodiments, the TLR7 agonist comprises 3-(5-amino-2-(4-(2-(3,3-difluoro-3-phosphonopropoxy)ethoxy)-2-methylphenethyl)benzo[f][1,7]naphthyridin-8-yl)propanoic acid. In some embodiments, the TLR7 agonist comprises a compound of formula:

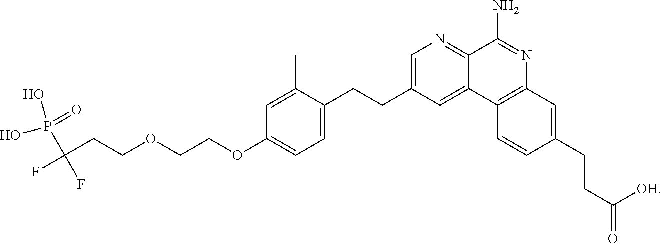 [SMILES: Cc1cc(OCCOCCC(F)(F)P(=O)(O)O)ccc1CCc1cnc2c(N)nc3cc(CCC(=O)O)ccc3c2c1]

In another embodiment, the ZBTB32 inhibitor, or a pharmaceutically acceptable salt, hydrate, solvate, prodrug, stereoisomer, or tautomer thereof, of the present disclosure are used in combination with one or more angiogenesis inhibitors to treat cancer, e.g., Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)₂-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aa,5p,6aa)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); or Aflibercept (Eylea®).

In another embodiment, the ZBTB32 inhibitor, or a pharmaceutically acceptable salt, hydrate, solvate, prodrug, stereoisomer, or tautomer thereof, of the present disclosure are used in combination with one or more heat shock protein inhibitors to treat cancer, e.g., Tanespimycin (17-allylamino-17-demethoxygeldanamycin, also known as KOS-953 and 17-AAG, available from SIGMA, and described in U.S. Pat. No. 4,261,989); Retaspimycin (IPI504), Ganetespib (STA-9090); [6-Chloro-9-(4-methoxy-3,5-dimethylpyridin-2-ylmethyl)-9H-purin-2-yl]amine (BIIB021 or —CNF2024, CAS 848695-25-0); trans-4-[[2-(Aminocarbonyl)-5-[4,5,6,7-tetrahydro-6,6-dimethyl-4-oxo-3-(trifluoromethyl)-1H-indazol-1-yl]phenyl]amino]cyclohexyl glycine ester (SNX5422 or PF04929113, CAS 908115-27-5); 5-[2,4-Dihydroxy-5-(1-methylethyl)phenyl]-N-ethyl-4-[4-(4-morpholinylmethyl)phenyl]-3-Isoxazolecarboxamide (AUY922, CAS 747412-49-3); or 17-Dimethylaminoethylamino-17-demethoxygeldanamycin (17-DMAG).

In yet another embodiment, the ZBTB32 inhibitor, or a pharmaceutically acceptable salt, hydrate, solvate, prodrug, stereoisomer, or tautomer thereof, of the present disclosure are used in combination with one or more HDAC inhibitors or other epigenetic modifiers. Exemplary HDAC inhibitors include, but not limited to, Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-η-oxo-2-oxiraneoctanoyl-O-methyl-D-tyrosyl-L-isoleucyl-L-prolyl] (Cyl-1); Cyclo[(αS,2S)-α-amino-η-oxo-2-oxiraneoctanoyl-O-methyl-D-tyrosyl-L-isoleucyl-(2S)-2-piperidinecarbonyl] (Cyl-2); Cyclic[L-alanyl-D-alanyl-(2S)-η-oxo-L-α-aminooxiraneoctanoyl-D-prolyl] (HC-toxin); Cyclo[(αS,2S)-α-amino-η-oxo-2-oxiraneoctanoyl-D-phenylalanyl-L-leucyl-(2S)-2-piperidinecarbonyl] (WF-3161); Chlamydocin ((S)-Cyclic(2-methylalanyl-L-phenylalanyl-D-prolyl-α-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); Depudecin (4,5:8,9-dianhydro-1,2,6,7,11-pentadeoxy-D-threo-D-ido-Undeca-1,6-dienitol); 4-(Acetylamino)-N-(2-aminophenyl)-benzamide (also known as CI-994); N1-(2-Aminophenyl)-N8-phenyl-octanediamide (also known as BML-210); 4-(Dimethylamino)-N-(7-(hydroxyamino)-7-oxoheptyl)benzamide (also known as M344); (E)-3-(4-(((2-(1H-indol-3-yl)ethyl)(2-hydroxyethyl)amino)-methyl)phenyl)-N-hydroxyacrylamide; Panobinostat(Farydak®); Mocetinostat, and Belinostat (also known as PXD101, Beleodaq®, or (2E)-N-Hydroxy-3-[3-(phenylsulfamoyl)phenyl]prop-2-enamide), or chidamide (also known as CS055 or HBI-8000, (E)-N-(2-amino-5-fluorophenyl)-4-((3-(pyridin-3-yl)acrylamido)methyl)benzamide). Other epigenetic modifiers include but not limited to inhibitors of EZH2 (enhancer of zeste homolog 2), EED (embryonic ectoderm development), or LSD1 (lysine-specific histone demethylase 1A or KDM1A).

In yet another embodiment, the ZBTB32 inhibitor, or a pharmaceutically acceptable salt, hydrate, solvate, prodrug, stereoisomer, or tautomer thereof, of the present disclosure are used in combination with one or more inhibitors of indoleamine-pyrrole 2,3-dioxygenase (IDO), for example, Indoximod (also known as NLG-8189), α-Cyclohexyl-5H-imidazo[5,1-a]isoindole-5-ethanol (also known as NLG919), or (4E)-4-[(3-Chloro-4-fluoroanilino)-nitrosomethylidene]-1,2,5-oxadiazol-3-amine (also known as INCB024360), to treat cancer.

### Chimeric Antigen Receptors

The present disclosure provides for the ZBTB32 inhibitor, or a pharmaceutically acceptable salt, hydrate, solvate, prodrug, stereoisomer, or tautomer thereof for use in combination with adoptive immunotherapy methods and reagents such as chimeric antigen receptor (CAR) immune effector cells, e.g., T cells, or chimeric TCR-transduced immune effector cells, e.g., T cells, as described herein.

### Estrogen Receptor Antagonists

In some embodiments, an estrogen receptor (ER) antagonist is used in combination with the ZBTB32 inhibitor, or a pharmaceutically acceptable salt, hydrate, solvate, prodrug, stereoisomer, or tautomer thereof, for treating a disease, e.g., cancer. In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signalling is implicated in cancers (e.g., breast cancer), e.g., ER positive (ER+) breast cancer. In some embodiments, the SERD is chosen from LSZ102, fulvestrant, brilanestrant, or elacestrant.

Exemplary Estrogen Receptor Antagonists

In some embodiments, the SERD comprises a compound disclosed in International Application Publication No. WO 2014/130310, which is hereby incorporated by reference in its entirety. In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid.

Other Exemplar Estrogen Receptor Antagonists

In some embodiments, the SERD comprises fulvestrant (CAS Registry Number: 129453-61-8), or a compound disclosed in International Application Publication No. WO 2001/051056, which is hereby incorporated by reference in its entirety. Fulvestrant is also known as ICI 182780, ZM 182780, FASLODEX®, or (7α,17β)-7-{9-[(4,4,5,5,5-pentafluoropentyl)sulfinyl]nonyl}estra-1,3,5(10)-triene-3,17-diol. Fulvestrant is a high affinity estrogen receptor antagonist with an IC50 of 0.29 nM.

In some embodiments, the SERD comprises elacestrant (CAS Registry Number: 722533-56-4), or a compound disclosed in U.S. Pat. No. 7,612,114, which is incorporated by reference in its entirety. Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56.

In some embodiments, the SERD is brilanestrant (CAS Registry Number: 1365888-06-7), or a compound disclosed in International Application Publication No. WO 2015/136017, which is incorporated by reference in its entirety. Brilanestrant is also known as GDC-0810, ARN810, RG-6046, RO-7056118 or (2E)-3-{4-[(1E)-2-(2-chloro-4-fluorophenyl)-1-(1H-indazol-5-yl)but-1-en-1-yl]phenyl}prop-2-enoic acid. Brilanestrant is a next-generation, orally bioavailable selective SERD with an IC50 of 0.7 nM. Brilanestrant is also disclosed, e.g., in Lai A. et al. (2015) Journal of Medicinal Chemistry 58 (12): 4888-4904.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887. Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety.

### CDK4/6 Inhibitors

In some embodiments, an inhibitor of Cyclin-Dependent Kinases 4 or 6 (CDK4/6) is used in combination with the ZBTB32 inhibitor, or a pharmaceutically acceptable salt, hydrate, solvate, prodrug, stereoisomer, or tautomer thereof, for treating a disease, e.g., cancer. In some embodiments, the CDK4/6 inhibitor is chosen from ribociclib, abemaciclib (Eli Lilly), or palbociclib.

### Exemplary CDK4/6 Inhibitors

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

Other Exemplary CDK4/6 Inhibitors

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine. Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

### CXCR2 Inhibitors

In some embodiments, an inhibitor of chemokine (C—X—C motif) receptor 2 (CXCR2) is used in combination with the ZBTB32 inhibitor, or a pharmaceutically acceptable salt, hydrate, solvate, prodrug, stereoisomer, or tautomer thereof, for treating a disease, e.g., cancer. In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

### Exemplary CXCR2 Inhibitors

In some embodiments, the CXCR2 inhibitor comprises a compound disclosed in U.S. Pat. Nos. 7,989,497, 8,288,588, 8,329,754, 8,722,925, 9,115,087, U.S. Application Publication Nos. US 2010/0152205, US 2011/0251205 and US 2011/0251206, and International Application Publication Nos. WO 2008/061740, WO 2008/061741, WO 2008/062026, WO 2009/106539, WO2010/063802, WO 2012/062713, WO 2013/168108, WO 2010/015613 and WO 2013/030803. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide choline salt. In some embodiments, the CXCR2 inhibitor is 2-Hydroxy-N,N,N-trimethylethan-1-aminium 3-chloro-6-({3,4-dioxo-2-[(pentan-3-yl)amino]cyclobut-1-en-1-yl}amino)-2-(N-methoxy-N-methylsulfamoyl)phenolate (i.e., 6-chloro-3-((3,4-dioxo-2-(pentan-3-ylamino)cyclobut-1-en-1-yl)amino)-2-hydroxy-N-methoxy-N-methylbenzenesulfonamide choline salt) and has the following chemical structure:

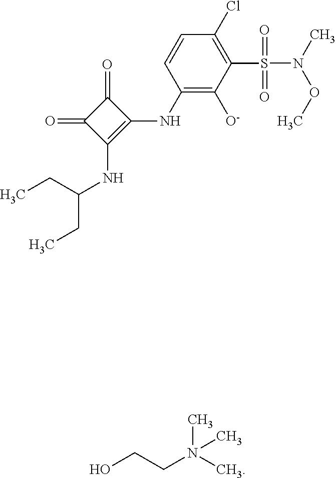

### Other Exemplary CXCR2 Inhibitors

In some embodiments, the CXCR2 inhibitor comprises danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea. Danirixin is disclosed, e.g., in Miller et al. Eur J Drug Metab Pharmacokinet (2014) 39:173-181; and Miller et al. BMC Pharmacology and Toxicology (2015), 16:18.

In some embodiments, the CXCR2 inhibitor comprises reparixin (CAS Registry Number: 266359-83-5). Reparixin is also known as repertaxin or (2R)-2-[4-(2-methylpropyl)phenyl]-N-methylsulfonylpropanamide. Reparixin is anon-competitive allosteric inhibitor of CXCR1/2. Reparixin is disclosed, e.g., in Zarbock et al. Br J Pharmacol. 2008; 155(3):357-64.

In some embodiments, the CXCR2 inhibitor comprises navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide. Navarixin is disclosed, e.g., in Ning et al. Mol Cancer Ther. 2012; 11(6):1353-64.

### CSF-1/1R Binding Agents

In some embodiments, a CSF-1/1R binding agent is used in combination with the ZBTB32 inhibitor, or a pharmaceutically acceptable salt, hydrate, solvate, prodrug, stereoisomer, or tautomer thereof, for treating a disease, e.g., cancer. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

### Exemplary CSF-1 Binding Agents

In some embodiments, the CSF-1/1R binding agent comprises an inhibitor of macrophage colony-stimulating factor (M-CSF). M-CSF is also sometimes known as CSF-1. In certain embodiments, the CSF-1/1R binding agent is an antibody to CSF-1 (e.g., MCS110). In other embodiments, the CSF-1/1R binding agent is an inhibitor of CSF-1R (e.g., BLZ945).

In some embodiments, the CSF-1/1R binding agent comprises a monoclonal antibody or Fab to M-CSF (e.g., MCS110/H—RX1), or a binding agent to CSF-1 disclosed in International Application Publication Nos. WO 2004/045532 and WO 2005/068503, including H—RX1 or 5H4 (e.g., an antibody molecule or Fab fragment against M-CSF) and U.S. Pat. No. 9,079,956, which applications and patent are incorporated by reference in their entirety.

TABLE 24
Amino acid and nucleotide sequences of an exemplary anti-M-CSF antibody molecule
(MCS110)

| | |
|---|---|
| (H-RX1) HC | QVQLQESGPGLVKPSQTLSLTCTVSDYSITSDYAWNWIRQFPGKGLEWMG |
| | YISYSGSTSYNPSLKSRITISRDTSKNQFSLQLNSVTAADTAVYYCASFDYA |
| | HAMDYWGQGTTVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK (SEQ ID NO: |
| | 271) |
| (H-RX1) LC | DIVLTQSPAFLSVTPGEKVTFTCQASQSIGTSIHWYQQKTDQAPKLLIKYAS |
| | ESISGIPSRFSGSGSGTDFTLTISSVEAEDAADYYCQQINSWPTTFGGGTKLEI |
| | KRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC (SEQ ID NO: 272) |
| Heavy Chain | SDYAWN (SEQ ID NO: 273) |
| CDR1 (Kabat) | |
| Heavy Chain | YISYSGSTSYNPSLKS (SEQ ID NO: 274) |
| CDR2 (Kabat) | |
| Heavy Chain | FDYAHAMDY (SEQ ID NO: 275) |
| CDR3 (Kabat) | |
| Light Chain | QASQSIGTSIH (SEQ ID NO: 276) |
| CDR1 (Kabat) | |
| Light Chain | YASESIS (SEQ ID NO: 277) |
| CDR2 (Kabat) | |
| Light Chain | QQINSWPTT (SEQ ID NO: 278) |
| CDR3 (Kabat) | |

In another embodiment, the CSF-1/1R binding agent comprises a CSF-1R tyrosine kinase inhibitor, 4-((2-(((1R,2R)-2-hydroxycyclohexyl)amino)benzo[d]thiazol-6-yl)oxy)-N-methyl picolinamide (BLZ945), or a compound disclosed in International Application Publication No. WO 2007/121484, and U.S. Pat. Nos. 7,553,854, 8,173,689, and 8,710,048, which are incorporated by reference in their entirety.

### Other Exemplary CSF-1/1R Binding Agents

In some embodiments, the CSF-1/1R binding agent comprises pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromethyl)pyridin-3-yl)methyl)pyridin-2-amine. Pexidartinib is a small-molecule receptor tyrosine kinase (RTK) inhibitor of KIT, CSF1R and FLT3. FLT3, CSF1R and FLT3 are overexpressed or mutated in many cancer cell types and play major roles in tumor cell proliferation and metastasis. PLX3397 can bind to and inhibit phosphorylation of stem cell factor receptor (KIT), colony-stimulating factor-1 receptor (CSF1R) and FMS-like tyrosine kinase 3 (FLT3), which may result in the inhibition of tumor cell proliferation and down-modulation of macrophages, osteoclasts and mast cells involved in the osteolytic metastatic disease.

In some embodiments, the CSF-1/1R binding agent is emactuzumab. Emactuzumab is also known as RG7155 or R05509554. Emactuzumab is a humanized IgG1 mAb targeting CSF1R. In some embodiments, the CSF-1/1R binding agent is FPA008. FPA008 is a humanized mAb that inhibits CSF1R.

### A2aR Antagonists

In some embodiments, an adenosine A2a receptor (A2aR) antagonist (e.g., an inhibitor of A2aR pathway, e.g., an adenosine inhibitor, e.g., an inhibitor of A2aR or CD-73) is used in combination with the ZBTB32 inhibitor, or a pharmaceutically acceptable salt, hydrate, solvate, prodrug, stereoisomer, or tautomer thereof, for treating a disease, e.g., cancer. In some embodiments, the A2aR antagonist is selected from PBF509 (NIR178) (Palobiofarma/Novartis), CP1444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), and Preladenant/SCH 420814 (Merck/Schering).

Exemplary A2aR antagonists

In some embodiments, the A2aR antagonist comprises PBF509 (NIR178) or a compound disclosed in U.S. Pat. No. 8,796,284 or in International Application Publication No. WO 2017/025918, herein incorporated by reference in their entirety. PBF509 (NIR178) is also known as NIR178.

### Other Exemplary A2aR Antagonists

In certain embodiments, the A2aR antagonist comprises CPI444/V81444. CPI-444 and other A2aR antagonists are disclosed in International Application Publication No. WO 2009/156737, herein incorporated by reference in its entirety. In certain embodiments, the A2aR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. In certain embodiments, the A2aR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2aR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine.

In certain embodiments, the A2aR antagonist is AZD4635/HTL-1071. A2aR antagonists are disclosed in International Application Publication No. WO 2011/095625, herein incorporated by reference in its entirety. In certain embodiments, the A2aR antagonist is 6-(2-chloro-6-methylpyridin-4-yl)-5-(4-fluorophenyl)-1,2,4-triazin-3-amine.

In certain embodiments, the A2aR antagonist is ST-4206 (Leadiant Biosciences). In certain embodiments, the A2aR antagonist is an A2aR antagonist described in U.S. Pat. No. 9,133,197, herein incorporated by reference in its entirety.

In certain embodiments, the A2aR antagonist is an A2aR antagonist described in U.S. Pat. Nos. 8,114,845 and 9,029,393, U.S. Application Publication Nos. 2017/0015758 and 2016/0129108, herein incorporated by reference in their entirety.

In some embodiments, the A2aR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-1H-purine-2,6-dione. Istradefylline is disclosed, e.g., in LeWitt et al. (2008) Annals of Neurology 63 (3): 295-302).

In some embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[4-[4-(2-methoxyethoxy)phenyl]-1-piperazinyl]ethyl]7H-pyrazolo[4,3-e][1,2,4]triazolo[1,5-c]pyrimidine-5-amine. Preladenant was developed as a drug that acted as a potent and selective antagonist at the adenosine A2A receptor.

In some embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine. Other exemplary A2aR antagonists include, e.g., ATL-444, MSX-3, SCH-58261, SCH-412,348, SCH-442,416, VER-6623, VER-6947, VER-7835, CGS-15943, and ZM-241,385.

In some embodiments, the A2aR antagonist is an A2aR pathway antagonist (e.g., a CD-73 inhibitor, e.g., an anti-CD73 antibody) is MEDI9447. MEDI9447 is a monoclonal antibody specific for CD73. Targeting the extracellular production of adenosine by CD73 may reduce the immunosuppressive effects of adenosine. MEDI9447 was reported to have a range of activities, e.g., inhibition of CD73 ectonucleotidase activity, relief from AMP-mediated lymphocyte suppression, and inhibition of syngeneic tumor growth. MEDI9447 can drive changes in both myeloid and lymphoid infiltrating leukocyte populations within the tumor microenvironment. These changes include, e.g., increases in CD8 effector cells and activated macrophages, as well as a reduction in the proportions of myeloid-derived suppressor cells (MDSC) and regulatory T lymphocytes.

### IDO Inhibitors

In some embodiments, an inhibitor of indoleamine 2,3-dioxygenase (IDO) and/or tryptophan 2,3-dioxygenase (TDO) is used in combination with the ZBTB32 inhibitor, or a pharmaceutically acceptable salt, hydrate, solvate, prodrug, stereoisomer, or tautomer thereof, for treating a disease, e.g., cancer. In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod ( ), (1-methyl-D-tryptophan), α-cyclohexyl-5H-Imidazo[5,1-a]isoindole-5-ethanol (also known as NLG919), indoximod, and BMS-986205 (formerly F001287).

### Exemplary IDO Inhibitors

In some embodiments, the IDO/TDO inhibitor is indoximod (New Link Genetics). Indoximod, the D isomer of 1-methyl-tryptophan, is an orally administered small-molecule indoleamine 2,3-dioxygenase (IDO) pathway inhibitor that disrupts the mechanisms by which tumors evade immune-mediated destruction.

In some embodiments, the IDO/TDO inhibitor is NLG919 (New Link Genetics). NLG919 is a potent IDO (indoleamine-(2,3)-dioxygenase) pathway inhibitor with Ki/EC50 of 7 nM/75 nM in cell-free assays.

In some embodiments, the IDO/TDO inhibitor is epacadostat (CAS Registry Number: 1204669-58-8). Epacadostat is also known as INCB24360 or INCB024360 (Incyte). Epacadostat is a potent and selective indoleamine 2,3-dioxygenase (IDO1) inhibitor with IC50 of 10 nM, highly selective over other related enzymes such as IDO2 or tryptophan 2,3-dioxygenase (TDO).

In some embodiments, the IDO/TDO inhibitor is F001287 (Flexus/BMS). F001287 is a small molecule inhibitor of indoleamine 2,3-dioxygenase 1 (IDO1).

### STING Agonists

In some embodiments, a STING agonist is used in combination with the ZBTB32 inhibitor, or a pharmaceutically acceptable salt, hydrate, solvate, prodrug, stereoisomer, or tautomer thereof, for treating a disease, e.g., cancer. In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage. In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

In some embodiments, the STING agonist is MK-1454 (Merck). MK-1454 is a cyclic dinucleotide Stimulator of Interferon Genes (STING) agonist that activates the STING pathway. Exemplary STING agonist are disclosed, e.g., in PCT Publication No. WO 2017/027645.

### Galectin Inhibitors

In some embodiments, a Galectin, e.g., Galectin-1 or Galectin-3, inhibitor is used in combination with the ZBTB32 inhibitor, or a pharmaceutically acceptable salt, hydrate, solvate, prodrug, stereoisomer, or tautomer thereof, for treating a disease, e.g., cancer. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck). Galectins are a family of proteins that bind to beta galactosidase sugars.

The Galectin family of proteins comprises at least of Galectin-1, Galectin-2, Galectin-3, Galectin-4, Galectin-7, and Galectin-8. Galectins are also referred to as S-type lectins, and are soluble proteins with, e.g., intracellular and extracellular functions.

Galectin-1 and Galectin-3 are highly expressed in various tumor types. Galectin-1 and Galectin-3 can promote angiogenesis and/or reprogram myeloid cells toward a pro-tumor phenotype, e.g., enhance immunosuppression from myeloid cells. Soluble Galectin-3 can also bind to and/or inactivate infiltrating T cells.

### Exemplary Galectin Inhibitors

In some embodiments, a Galectin inhibitor is an antibody molecule. In an embodiment, an antibody molecule is a monospecific antibody molecule and binds a single epitope. E.g., a monospecific antibody molecule having a plurality of immunoglobulin variable domain sequences, each of which binds the same epitope. In an embodiment, the Galectin inhibitor is an anti-Galectin, e.g., anti-Galectin-1 or anti-Galectin-3, antibody molecule. In some embodiments, the Galectin inhibitor is an anti-Galectin-1 antibody molecule. In some embodiments, the Galectin inhibitor is an anti-Galectin-3 antibody molecule.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, the Galectin inhibitor is a multispecific antibody molecule. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the Galectin inhibitor is a bispecific antibody molecule. In an embodiment, the first epitope is located on Galectin-1, and the second epitope is located on Galectin-3.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH—CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In other embodiments, the anti-Galectin, e.g., anti-Galectin-1 or anti-Galectin-3, antibody molecule (e.g., a monospecific, bispecific, or multispecific antibody molecule) is covalently linked, e.g., fused, to another partner e.g., a protein, e.g., as a fusion molecule for example a fusion protein. In one embodiment, a bispecific antibody molecule has a first binding specificity to a first target (e.g., to Galectin-1), a second binding specificity to a second target (e.g., Galectin-3).

This disclosure provides an isolated nucleic acid molecule encoding the above antibody molecule, vectors and host cells thereof. The nucleic acid molecule includes but is not limited to RNA, genomic DNA and cDNA.

In some embodiments, a Galectin inhibitor is a peptide, e.g., protein, which can bind to, and inhibit Galectin, e.g., Galectin-1 or Galectin-3, function. In some embodiments, the Galectin inhibitor is a peptide which can bind to, and inhibit Galectin-3 function. In some embodiments, the Galectin inhibitor is the peptide Galectin-3C. In some embodiments, the Galectin inhibitor is a Galectin-3 inhibitor disclosed in U.S. Pat. No. 6,770,622, which is hereby incorporated by reference in its entirety.

Galectin-3C is an N-terminal truncated protein of Galectin-3, and functions, e.g., as a competitive inhibitor of Galectin-3. Galectin-3C prevents binding of endogenous Galectin-3 to e.g., laminin on the surface of, e.g., cancer cells, and other beta-galactosidase glycoconjugates in the extracellular matrix (ECM). Galectin-3C and other exemplary Galectin inhibiting peptides are disclosed in U.S. Pat. No. 6,770,622.

In some embodiments, Galectin-3C comprises the amino acid sequence of SEQ ID NO: 279, or an amino acid substantially identical (e.g., 90, 95 or 99%) identical thereto.

(SEQ ID NO: 279)
GAPAGPLIVPYNLPLPGGVVPRMLITILGTVKPNANRIALDFQRGNDVAF
HFNPRFNENNRRVIVCNTKLDNNWGREERQSVFPFESGKPFKIQVLVEPD
HFKVAVNDAHLLQYNHRVKKLNEISKLGISGDIDITSASYTMI

In some embodiments, the Galectin inhibitor is a peptide, which can bind to, and inhibit Galectin-1 function. In some embodiments, the Galectin inhibitor is the peptide Anginex: Anginex is an anti-angiongenic peptide that binds Galectin-1 (Salomonsson E, et al., (2011) Journal of Biological Chemistry, 286(16):13801-13804). Binding of Anginex to Galectin-1 can interfere with, e.g., the pro-angiongenic effects of Galectin-1.

In some embodiments, the Galectin, e.g., Galectin-1 or Galectin-3, inhibitor is a non-peptidic topomimetic molecule. In some embodiments, the non-peptidic topomimetic Galectin inhibitor is OTX-008 (OncoEthix). In some embodiments, the non-peptidic topomimetic is a non-peptidic topomimetic disclosed in U.S. Pat. No. 8,207,228, which is herein incorporated by reference in its entirety. OTX-008, also known as PTX-008 or Calixarene 0118, is a selective allosteric inhibitor of Galectin-1. OTX-008 has the chemical name: N-[2-(dimethylamino)ethyl]-2-{[26,27,28-tris({[2-(dimethylamino)ethyl]carbamoyl}methoxy) pentacyclo[19.3.1.1,7.1,0.15,]octacosa-1(25),3(28),4,6,9(27),1012,15,17,19(26),21,23-dodecaen-25-yl]oxy}acetamide.

In some embodiments, the Galectin, e.g., Galectin-1 or Galectin-3, inhibitor is a carbohydrate based compound. In some embodiments, the Galectin inhibitor is GR-MD-02 (Galectin Therapeutics). In some embodiments, GR-MD-02 is a Galectin-3 inhibitor. GR-MD-02 is a galactose-pronged polysaccharide also referred to as, e.g., a galactoarabino-rhamnogalaturonate. GR-MD-02 and other galactose-pronged polymers, e.g., galactoarabino-rhamnogalaturonates, are disclosed in U.S. Pat. No. 8,236,780 and U.S. Publication 2014/0086932, the entire contents of which are herein incorporated by reference in their entirety.

### MEK Inhibitors

In some embodiments, a MEK inhibitor is used in combination with the ZBTB32 inhibitor, or a pharmaceutically acceptable salt, hydrate, solvate, prodrug, stereoisomer, or tautomer thereof, for treating a disease, e.g., cancer. In some embodiments, the MEK inhibitor is chosen from Trametinib, selumetinib, AS703026, BIX 02189, BIX 02188, CI-1040, PD0325901, PD98059, U0126, XL-518, G-38963, or G02443714. In some embodiments, the MEK inhibitor is Trametinib.

Exemplary MEK Inhibitors

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3).

### Other Exemplary MEK Inhibitors

In some embodiments the MEK inhibitor comprises selumetinib which has the chemical name: (5-[(4-bromo-2-chlorophenyl)amino]-4-fluoro-N-(2-hydroxyethoxy)-1-methyl-1H-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a CAS No. 1029872-29-4 and is available from ACC Corp.

In some embodiments, the MEK inhibitor comprises G-38963.

In some embodiments, the MEK inhibitor comprises G02443714 (also known as AS703206)

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

c-MET Inhibitors

In some embodiments, a c-MET inhibitor is used in combination with the ZBTB32 inhibitor, or a pharmaceutically acceptable salt, hydrate, solvate, prodrug, stereoisomer, or tautomer thereof, for treating a disease, e.g., cancer. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein.

In some embodiments, the c-MET inhibitor is chosen from capmatinib (INC280), JNJ-3887605, AMG 337, LY2801653, MSC2156119J, crizotinib, tivantinib, or golvatinib.

Exemplary c-MET Inhibitors

In some embodiments, the c-MET inhibitor comprises capmatinib (INC280), or a compound described in U.S. Pat. Nos. 7,767,675, and 8,461,330, which are incorporated by reference in their entirety.

Other Exemplary c-MET Inhibitors

In some embodiments, the c-MET inhibitor comprises JNJ-38877605. JNJ-38877605 is an orally available, small molecule inhibitor of c-Met. JNJ-38877605 selectively binds to c-MET, thereby inhibiting c-MET phosphorylation and disrupting c-Met signal transduction pathways. In some embodiments, the c-Met inhibitor is AMG 208. AMG 208 is a selective small-molecule inhibitor of c-MET. AMG 208 inhibits the ligand-dependent and ligand-independent activation of c-MET, inhibiting its tyrosine kinase activity, which may result in cell growth inhibition in tumors that overexpress c-Met.

In some embodiments, the c-Met inhibitor comprises AMG 337. AMG 337 is an orally bioavailable inhibitor of c-Met. AMG 337 selectively binds to c-MET, thereby disrupting c-MET signal transduction pathways.

In some embodiments, the c-Met inhibitor comprises LY2801653. LY2801653 is an orally available, small molecule inhibitor of c-Met. LY2801653 selectively binds to c-MET, thereby inhibiting c-MET phosphorylation and disrupting c-Met signal transduction pathways.

In some embodiments, c-Met inhibitor comprises MSC2156119J. MSC2156119J is an orally bioavailable inhibitor of c-Met. MSC2156119J selectively binds to c-MET, which inhibits c-MET phosphorylation and disrupts c-Met-mediated signal transduction pathways.

In some embodiments, the c-MET inhibitor is capmatinib. Capmatinib is also known as INCB028060. Capmatinib is an orally bioavailable inhibitor of c-MET. Capmatinib selectively binds to c-Met, thereby inhibiting c-Met phosphorylation and disrupting c-Met signal transduction pathways.

In some embodiments, the c-MET inhibitor comprises crizotinib. Crizotinib is also known as PF-02341066. Crizotinib is an orally available aminopyridine-based inhibitor of the receptor tyrosine kinase anaplastic lymphoma kinase (ALK) and the c-Met/hepatocyte growth factor receptor (HGFR). Crizotinib, in an ATP-competitive manner, binds to and inhibits ALK kinase and ALK fusion proteins. In addition, crizotinib inhibits c-Met kinase, and disrupts the c-Met signalling pathway. Altogether, this agent inhibits tumor cell growth.

In some embodiments, the c-MET inhibitor comprises golvatinib. Golvatinib is an orally bioavailable dual kinase inhibitor of c-MET and VEGFR-2 with potential antineoplastic activity. Golvatinib binds to and inhibits the activities of both c-MET and VEGFR-2, which may inhibit tumor cell growth and survival of tumor cells that overexpress these receptor tyrosine kinases.

In some embodiments, the c-MET inhibitor is tivantinib. Tivantinib is also known as ARQ 197. Tivantinib is an orally bioavailable small molecule inhibitor of c-MET. Tivantinib binds to the c-MET protein and disrupts c-Met signal transduction pathways, which may induce cell death in tumor cells overexpressing c-MET protein or expressing constitutively activated c-Met protein.

### TGF-β Inhibitors

In some embodiments, a transforming growth factor beta (also known as TGF-β TGFβ, TGFb, or TGF-beta, used interchangeably herein) inhibitor is used in combination with the ZBTB32 inhibitor, or a pharmaceutically acceptable salt, hydrate, solvate, prodrug, stereoisomer, or tautomer thereof, for treating a disease, e.g., cancer. In certain embodiments, a combination described herein comprises a transforming growth factor beta (also known as TGF-β TGFβ, TGFb, or TGF-beta, used interchangeably herein) inhibitor.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β 1, TGF-β2, or TGF-β3).

Under normal conditions, TGF-β maintains homeostasis and limits the growth of epithelial, endothelial, neuronal and hematopoietic cell lineages, e.g., through the induction of anti-proliferative and apoptotic responses. Canonical and non-canonical signalling pathways are involved in cellular responses to TGF-β. Activation of the TGF-f/Smad canonical pathway can mediate the anti-proliferative effects of TGF-β. The non-canonical TGF-β pathway can activate additional intra-cellular pathways, e.g., mitogen-activated protein kinases (MAPK), phosphatidylinositol 3 kinase/Protein Kinase B, Rho-like GTPases (Tian et al. Cell Signal. 2011; 23(6):951-62; Blobe et al. N Engl J Med. 2000; 342(18):1350-8), thus modulating epithelial to mesenchymal transition (EMT) and/or cell motility.

Alterations of TGF-β signalling pathway are associated with human diseases, e.g., cancers, cardio-vascular diseases, fibrosis, reproductive disorders, and wound healing. Without wishing to be bound by theory, it is believed that in some embodiments, the role of TGF-β in cancer is dependent on the disease setting (e.g., tumor stage and genetic alteration) and/or cellular context. For example, in late stages of cancer, TGF-β can modulate a cancer-related process, e.g., by promoting tumor growth (e.g., inducing EMT), blocking anti-tumor immune responses, increasing tumor-associated fibrosis, or enhancing angiogenesis (Wakefield and Hill Nat Rev Cancer. 2013; 13(5):328-41). In certain embodiments, a combination comprising a TGF-β inhibitor described herein is used to treat a cancer in a late stage, a metastatic cancer, or an advanced cancer.

Preclinical evidence indicates that TGF-β plays an important role in immune regulation (Wojtowicz-Praga Invest New Drugs. 2003; 21(1):21-32; Yang et al. Trends Immunol. 2010; 31(6):220-7). TGF-β can down-regulate the host-immune response via several mechanisms, e.g., shift of the T-helper balance toward Th2 μmmune phenotype; inhibition of anti-tumoral Th1 type response and M1-type macrophages; suppression of cytotoxic CD8+ T lymphocytes (CTL), NK lymphocytes and dendritic cell functions, generation of CD4⁺ CD25⁺ T-regulatory cells; or promotion of M2-type macrophages with pro-tumoral activity mediated by secretion of immunosuppressive cytokines (e.g., IL10 or VEGF), pro-inflammatory cytokines (e.g., IL6, TNFα, or IL1) and generation of reactive oxygen species (ROS) with genotoxic activity (Yang et al. Trends Immunol. 2010; 31(6):220-7; Truty and Urrutia Pancreatology. 2007; 7(5-6):423-35; Achyut et al Gastroenterology. 2011; 141(4):1167-78).

### Exemplary TGF-β Inhibitors

In some embodiments, the TGF-β inhibitor comprises XOMA 089, or a compound disclosed in International Application Publication No. WO 2012/167143, which is incorporated by reference in its entirety.

XOMA 089 is also known as XPA.42.089. XOMA 089 is a fully human monoclonal antibody that specifically binds and neutralizes TGF-beta 1 and 2 ligands.

The heavy chain variable region of XOMA 089 has the amino acid sequence of:

(SEQ ID NO: 284)
QVQLVQSGAEVKKPGSSVKVSCKASGGTFSSYAISWVRQAPGQGLEWMGG
IIPIFGTANYAQKFQGRVTITADESTSTAYMELSSLRSEDTAVYYCARGL
WEVRALPSVYWGQGTLVTVSS (disclosed as SEQ ID NO: 6
in WO 2012/167143).

The light chain variable region of XOMA 089 has the amino acid sequence of:

(SEQ ID NO: 285)
SYELTQPPSVSVAPGQTARITCGANDIGSKSVHWYQQKAGQAPVLVVSED
IIRPSGIPERISGSNSGNTATLTISRVEAGDEADYYCQVWDRDSDQYVFG
TGTKVTVLG (disclosed as SEQ ID NO: 8 in WO
2012/167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 pM for TGF-β1, 67.3 pM for TGF-β2, and 948 pM for TGF-β3. Given the high affinity binding to all three TGF-β isoforms, in certain embodiments, XOMA 089 is expected to bind to TGF-β1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

### Other Exemplary TGF-β Inhibitors

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of

(SEQ ID NO: 280)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
HDGSFFLYSRLTVDKSRWQEGNVFSCSVMHEALHNYTQKSLSLSLGK.

The light chain of fresolimumab has the amino acid sequence of:

(SEQ ID NO: 281)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

Fresolimumab is disclosed, e.g., in International Application Publication No. WO 2006/086469, and U.S. Pat. Nos. 8,383,780 and 8,591,901, which are incorporated by reference in their entirety.

### IL-1β Inhibitors

The Interleukin-1 (IL-1) family of cytokines is a group of secreted pleotropic cytokines with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). The IL-1 family comprises, inter alia, IL-1 beta (IL-1b), and IL-1alpha (IL-1a). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc. Natl. Acad. Sci. U.S.A. p. 2645-50).

In some embodiments, an interleukin-1 beta (IL-1β) inhibitor is used in combination with the ZBTB32 inhibitor, or a pharmaceutically acceptable salt, hydrate, solvate, prodrug, stereoisomer, or tautomer thereof, for treating a disease, e.g., cancer. In some embodiments, the IL-1β inhibitor is chosen from canakinumab, gevokizumab, Anakinra, or Rilonacept. In some embodiments, the IL-1β inhibitor is canakinumab.

### Exemplary IL-1β Inhibitors

In some embodiments, the IL-1β inhibitor is canakinumab. Canakinumab is also known as ACZ885 or ILARIS®. Canakinumab is a human monoclonal IgG1/κ antibody that neutralizes the bioactivity of human IL-1β.

Canakinumab is disclosed, e.g., in WO 2002/16436, U.S. Pat. No. 7,446,175, and EP 1313769. The heavy chain variable region of canakinumab has the amino acid sequence of:

(SEQ ID NO: 282)
MEFGLSWVFLVALLRGVQCQVQLVESGGGVVQPGRSLRLSCAASGFTFSV
YGMNWVRQAPGKGLEWVAIIWYDGDNQYYADSVKGRFTISRDNSKNTLYL
QMNGLRAEDTAVYYCARDLRTGPFDYWGQGTLVTVSS (disclosed
as SEQ ID NO: 1 in U.S. Pat. No. 7,446,175).

The light chain variable region of canakinumab has the amino acid sequence of:

(SEQ ID NO: 283)
MLPSQLIGFLLLWVPASRGEIVLTQSPDFQSVTPKEKVTITCRASQSIGS
SLHWYQQKPDQSPKLLIKYASQSFSGVPSRFSGSGSGTDFTLTINSLEAE
DAAAYYCHQSSSLPFTFGPGTKVDIK (disclosed as SEQ ID
NO: 2 in U.S. Pat. No. 7,446,175).

Canakinumab has been used, e.g., for the treatment of Cryopyrin Associated Periodic Syndromes (CAPS), in adults and children, for the treatment of systemic juvenile idiopathic arthritis (SJIA), for the symptomatic treatment of acute gouty arthritis attacks in adults, and for other IL-1 driven inflammatory diseases. Without wishing to be bound by theory, it is believed that in some embodiments, IL-1β inhibitors, e.g., canakinumab, can increase anti-tumor immune response, e.g., by blocking one or more functions of IL-1b including, e.g., recruitment of immunosuppressive neutrophils to the tumor microenvironment, stimulation of tumor angiogenesis, and/or promotion of metastasis (Dinarello (2010) Eur. J Immunol. p. 599-606).

In some embodiments, the combination described herein includes an IL-1β inhibitor, canakinumab, or a compound disclosed in WO 2002/16436, and an inhibitor of an immune checkpoint molecule, e.g., an inhibitor of PD-1 (e.g., an anti-PD-1 antibody molecule). IL-1 is a secreted pleotropic cytokine with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc. Natl. Acad. Sci. U.S.A. p. 2645-50). Canakinumab can bind IL-1b and inhibit IL-1-mediated signalling. Accordingly, in certain embodiments, an IL-1β inhibitor, e.g., canakinumab, enhances, or is used to enhance, an immune-mediated anti-tumor effect of an inhibitor of PD-1 (e.g., an anti-PD-1 antibody molecule).

In some embodiments, the IL-1β inhibitor, canakinumab, or a compound disclosed in WO 2002/16436, and the inhibitor of an immune checkpoint molecule, e.g., an inhibitor of PD-1 (e.g., an anti-PD-1 antibody molecule), each is administered at a dose and/or on a time schedule, that in combination, achieves a desired anti-tumor activity.

MDM2 inhibitors

In some embodiments, a mouse double minute 2 homolog (MDM2) inhibitor is used in combination with the ZBTB32 inhibitor, or a pharmaceutically acceptable salt, hydrate, solvate, prodrug, stereoisomer, or tautomer thereof, for treating a disease, e.g., cancer. The human homolog of MDM2 is also known as HDM2. In some embodiments, an MDM2 inhibitor described herein is also known as a HDM2 inhibitor. In some embodiments, the MDM2 inhibitor is chosen from HDM201 or CGM097.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In an embodiment, an MDM2 inhibitor comprises an inhibitor of p53 and/or a p53/Mdm2 interaction. In an embodiment, the MDM2 inhibitor comprises (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (also known as HDM201), or a compound disclosed in PCT Publication No. WO2013/111105 to treat a disorder, e.g., a disorder described herein. In one embodiment, a therapeutic agent disclosed herein is used in combination with HDM201. In some embodiments, HDM201 is administered orally.

In one embodiment, the combination disclosed herein is suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest.

### Multispecific Binding Molecules

In some embodiments, the ZBTB32 inhibitor, or a pharmaceutically acceptable salt, hydrate, solvate, prodrug, stereoisomer, or tautomer thereof, is used in combination with a multispecific binding molecule (“MBM”). As used herein, the term “MBM” refers to a binding molecule that recognizes two or more different epitopes. Examples of MBMs include bispecific binding molecules (“BBMs”), which recognize two different epitopes, and trispecific binding molecules (“TBMs”), which recognize three different epitopes. The epitopes can be present on the same target or on different targets.

The MBMs suitable for use or administration in combination with the ZBTB32 inhibitors of the disclosure thus comprise at least two antigen binding domains (“ABDs”) that bind to different epitopes. The term “antigen-binding domain” or “ABD” as used herein refers to a portion of an MBM that has the ability to bind to an epitope non-covalently, reversibly and specifically.

Generally, for treatment of cancer, the MBMs useful in combination with the ZBTB32 inhibitors of the disclosure bind to at least one tumor-associated antigen (“TAA”). As used herein, the term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. A TAA is a marker that may be expressed by both normal cells and cancer cells, e.g., a lineage marker, e.g., CD19 on B cells. A TAA may also be a cell surface molecule that is overexpressed in a cancer cell in comparison to a normal cell, for instance, 1-fold overexpression, 2-fold overexpression, 3-fold overexpression or more in comparison to a normal cell. A TAA may be a cell surface molecule that is inappropriately synthesized in the cancer cell, for instance, a molecule that contains deletions, additions or mutations in comparison to the molecule expressed on a normal cell. Certain TAAs may be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”). Further, as used herein, the term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, leukemia, multiple myeloma, asymptomatic myeloma, Hodgkin's lymphoma and non-Hodgkin's lymphoma. In some embodiments, the TAA is expressed on cancerous B cells. The term “cancerous B cell” refers to a B cell that is undergoing or has undergone uncontrolled proliferation.

Examples of TAAs that can be targeted by the MBMs (e.g., BBMs or TBMs) useful in combination with the ZBTB32 inhibitors of the disclosure include TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, EGFRvIII, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, FLT3, TAAG72, CD22, CD33, GD2, BCMA, gp100Tn, FAP, tyrosinase, EPCAM, CEA, Igf-I receptor, Cadherin17, CD32b, GPNMB, GPR64, HER3, LRP6, LYPD8, NKG2D, SLC34A2, SLC39A6, SLITRK6, TACSTD2, and EphB2.

In certain aspects, the TAAs are expressed on cancerous blood cells, e.g., cancerous B cells. Examples of TAAs expressed on cancerous B cells include, but are not limited to, CD19, CD20, CD22, CD123, BCMA, CD33, CLL1, CD138 (also known as Syndecan-1, SDC1), CS1, CD38, CD133, FLT3, CD52, TNFRSF13C (TNF Receptor Superfamily Member 13C, also referred to in the art as BAFFR: B-Cell-Activating Factor Receptor), TNFRSF13B (TNF Receptor Superfamily Member 13B, also referred to in the art as TACI: Transmembrane Activator And CAML Interactor), CXCR4 (C—X—C Motif Chemokine Receptor 4), PD-L1 (programmed death-ligand 1), LY9 (lymphocyte antigen 9, also referred to in the art as CD229), CD200, FCGR2B (Fc fragment of IgG receptor lib, also referred to in the art as CD32b), CD21, CD23, CD24, CD40L, CD72, CD79a, and CD79b.

In addition to binding a TAA, MBMs useful in combination with the ZBTB32 inhibitors of the disclosure can engage the immune system, for example a T cell or an NK cell. Engagement of T cells can be achieved through targeting CD3 or other component(s) of a TCR complex, for example TCR-α, TCR-β, or a TCR-α/β dimer. Exemplary ABDs that recognize CD3 or other components of the TCR complex are described in WO 2020/052692 and WO2019/104075 (for example see Sections 7.8.1, 7.8.2 and 7.8.3 of WO 2020/052692 and Section 6.5 of WO 2019/104075, incorporated by reference herein). The MBMs can further include an ABD that binds to CD2, for example as generally disclosed in WO 2019/104075. In some embodiments, CD2 can be targeted through the use of its ligand CD58 and CD2-binding portions thereof as ABDs, as described in Section 6.6.2 of WO 2019/104075, incorporated by reference herein. Engagement of NK cells can be achieved through targeting CD16, NKp46, NKG2D, NKp30, NKp44, NKp46, or a combination thereof, e.g., a combination of CD16 and NKp46. See, e.g., Hu et al., 2019, Front. Immunol. 10:1205, Gauthier et al., 2019, Cell 177(7):1701-1713.

In some embodiments, the MBM is a BBM that binds to B cell maturation antigen, or BCMA, and a component of the TCR complex, preferably CD3. In other embodiments, the MBM is a TBM that binds to BCMA, a component of the TCR complex, preferably CD3, as well as either a second TAA or CD2 (e.g., through a CD58-based ABD, e.g., an ABD containing amino acids residues 30-123 of CD58). The expression of BCMA has been linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma. WO 2019/229701 describes a number of MBMs that specifically bind to human BCMA as well as sequences of exemplary BCMA binding sequences that can be included in MBMs that bind to BCMA (see for example the BCMA binding sequences set disclosed in paragraph [0149] and Table 1 of WO 2019/229701, incorporated by reference herein). WO 2019/229701 also describes BBMs that are directed against BCMA and CD3 (see for example Section 7.3.3.1 of PCT WO 2019/229701, incorporated by reference herein).

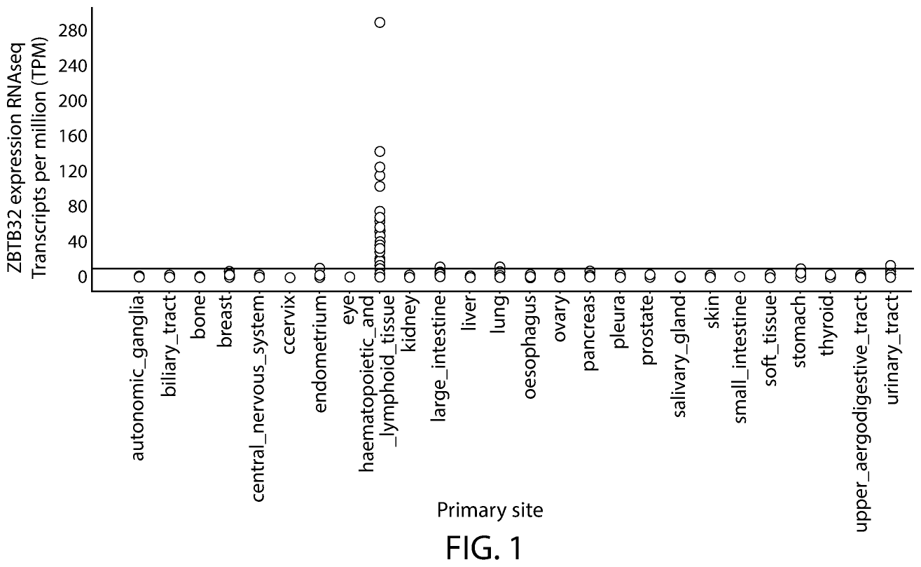

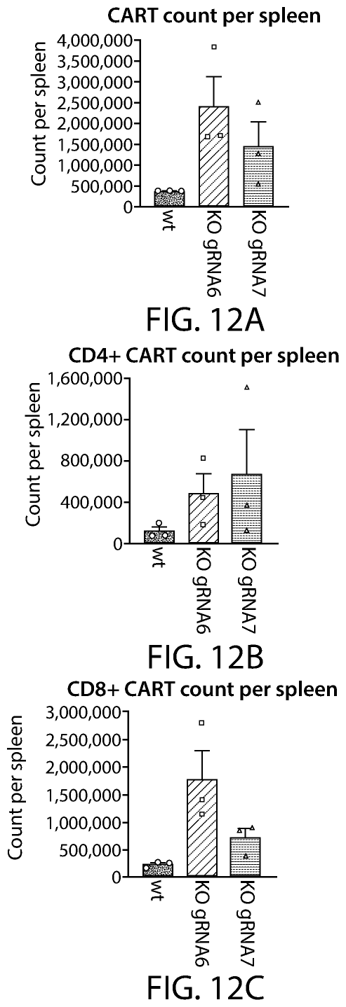

In other embodiments, the MBM is a BBM that binds CD19 and a component of the TCR complex, preferably CD3. In other embodiments, the MBM is a TBM that binds to CD19, a component of the TCR complex, preferably CD3, as well as either a second TAA or CD2 (e.g., through a CD58-based ABD, e.g., an ABD containing amino acids residues 30-123 of CD58). A TBM that binds to CD19, CD3 and CD2 (e.g., through a CD58-based ABD, e.g., an ABD containing amino acids residues 30-123 of CD58) can have the general configuration depicted in FIG. 1D of WO2019/104075, for example where X in FIG. 1D is a CD19 ABD, X is a CD3 ABM and Z is a CD2 ABM, and more specifically the configurations shown in FIGS. 12A, 12B and 12C of WO2019/104075, all of which figures and accompanying text are incorporated by reference herein. CD19 is expressed during early pre-B cell development and remains until plasma cell differentiation. CD19 is expressed on both normal B cells and malignant B cells whose abnormal growth can lead to B-cell lymphomas. For example, CD19 is expressed on B-cell lineage malignancies, including, but not limited to non-Hodgkin's lymphoma (B-NHL), chronic lymphocytic leukemia, and acute lymphoblastic leukemia.

Whilst a large number of MBMs that are being developed for cancer therapy typically include one or more ABDs directed against a TAA and one or more ABDs that are able to facilitate engagement of immune cells, MBMs useful in combination with the ZBTB32 inhibitors of the disclosure need not engage immune cells such as T cells or NK cells. For example, MBMs can be used to inhibit the angiogenesis pathways, for example by targeting VEG-F and another antigen such as Delta-like Ligand 4 (DLL-4), a transmembrane ligand for the Notch receptor or angiopoietin 2 (ANG-2).

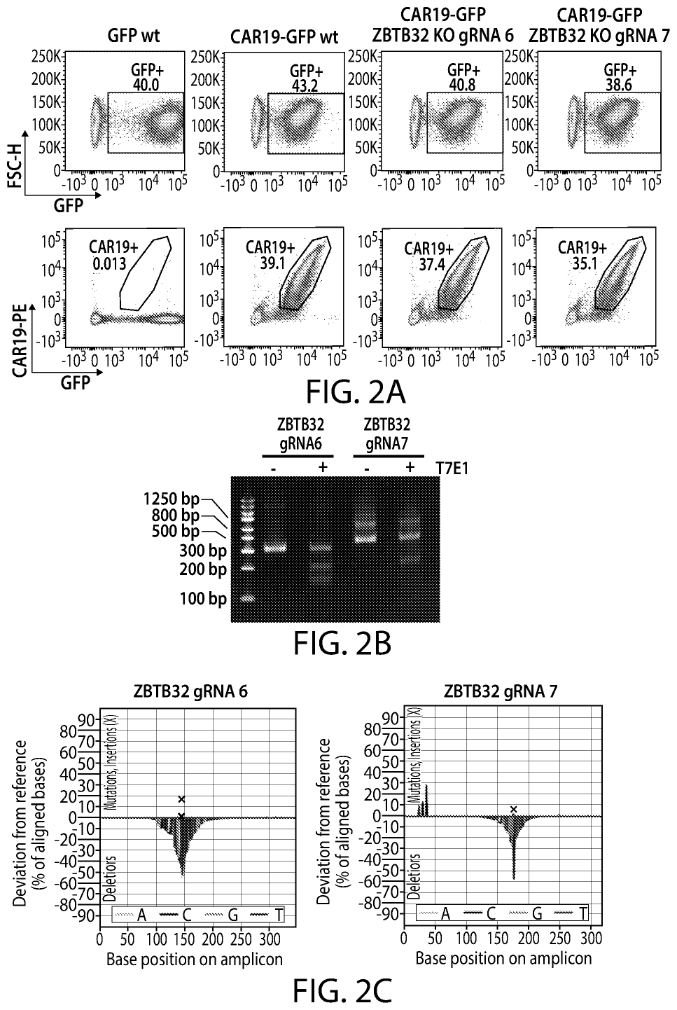

Exemplary types of ABDs include antigen-binding fragments and portions of both immunoglobulin and non-immunoglobulin based scaffolds that retain the ability of binding an antigen non-covalently, reversibly and specifically. Thus, as used herein, the term “antigen-binding domain” encompasses antibody fragments that retain the ability of binding an antigen non-covalently, reversibly and specifically. Examples of binding fragments include, but are not limited to, single-chain Fvs (scFv), a Fab fragment, a monovalent fragment consisting of the VL, VH, CL and CH1 domains; a Fd fragment consisting of the VH and CH1 domains; a Fv fragment consisting of the VL and VH domains of a single arm of an antibody; a dAb fragment which consists of a VH domain; and an isolated complementarity determining region (CDR). Thus, the term “antibody fragment” encompasses both proteolytic fragments of antibodies (e.g., Fab and F(ab)₂ fragments) and engineered proteins comprising one or more portions of an antibody (e.g., an scFv). In addition to immunoglobulin-based ABDs, such Fab-, scFv- and other antibody fragment based ABDs such as those described above), the MBMs of the disclosure can also include non-immunoglobulin-based ABDs, or a combination of immunoglobulin and non-immunoglobulin based ABDs. Immunoglobulin-based ABDs that can be used are described in WO 2019/104075 and WO 2019/229701 (see for example Sections 7.2 and 7.3.1 of WO 2019/229701 and Section 6.2.1 of WO 2019/104075, incorporated by reference herein). Non-immunoglobulin-based ABDs, which include Kunitz domains, Adnexins, Affibodies, DARPins, Avimers, Anticalins, Lipocalins, fibronectins scaffolds, Affimers, and Fynomers, are described in WO 2019/104075 and WO 2019/229701 (see for example Section 7.4 of WO 2019/229701 and Section 6.3 of WO 2019/104075 and FIG. 2 and Table 1 of Hober et al., 2019, Methods 154:143-152, incorporated by reference herein). For MBMs that target CD2, a suitable ABD is CD58 or a fragment thereof, as described in Section 6.6.2 of WO 2019/104075, incorporated by reference herein.

BBMs comprise at least two ABDs but can also contain more than two ABDs. BBMs that contain only two ABDs and are considered bivalent, and a BBM can have three ABDs (i.e., is trivalent), four ABDs (i.e., is tetravalent), or more, provided that the BBM has at least one ABD that can bind one epitope and at least one ABD that can bind a different epitope. Exemplary bivalent, trivalent, and tetravalent BBM configurations are described in WO 2019/229701 (for example FIG. 1 and Section 7.5 of WO 2019/229701 and Section 7.5 of WO 2019/229701, incorporated by reference herein). Although WO 2019/229701 relates to BCMA binding molecules, which can suitably be used in combination with the ZBTB32 inhibitors of the disclosure, the BBM formats described therein are also applicable to any epitope/antigen pairing, for example any TAA and T cell receptor component pair or a TAA and NK cell activating receptor such as CD16, NKp46, NKG2D, NKp30, NKp44 or NKp46. Similarly, TBMs have at least three ABDs (i.e., a TBM is at least trivalent), but can also contain more than three ABDs. For example, a TBM can have four ABDs (i.e., is tetravalent), five ABDs (i.e., is pentavalent), or six ABDs (i.e., is hexavalent), provided that the TBM has at least one ABD that can bind one epitope, at least one ABD that can bind a second epitope, and at least one ABD that can bind a third epitope. Exemplary trivalent, tetravalent, pentavalent, and hexavalent TBM configurations are described in WO 2019/104075 and WO 2019/195535 (for example FIG. 1 and Section 6.4 of WO 2019/104075 and FIG. 1 and Section 7.4 of WO 2019/195535, incorporated by reference herein). Although WO 2019/104075 relates to TBMs that bind to CD2, CD3 (or another T cell receptor component) and a tumor-associated antigen (TAA) and WO 2019/195535 relates to TBMs that bind to CD3 (or another T cell receptor component) and two TAAs, both of which can suitably be used in combination with the ZBTB32 inhibitors of the disclosure, the TBM formats described therein are also applicable to any combination of three epitopes or antigens. For example, a TBM can target a TAA and two NK cell activating receptors, for example any two of CD16, NKp46, NKG2D, NKp30, NKp44 or NKp46. In a particular embodiment, a TBM useful in combination with the ZBTB32 inhibitors of the disclosure can target a TAA, CD16 and NKp46 (see, e.g., Gauthier et al., 2019, Cell 177(7):1701-1713).

The ABDs of an MBM (or portions thereof) can be connected to each other, for example, by short peptide linkers or by an Fc domain. Methods of connecting ABDs to form an MBM are described in WO 2019/104075 and WO 2019/229701 (see for example Section 7.3.2 of WO 2019/229701 and Section 6.2.2 of WO 2019/104075, incorporated by reference herein).

The MBMs can have an Fc region formed by the association of two Fc domains. The Fc domains can be homodimeric or heterodimeric. Exemplary heterodimerization strategies, which include knob-into-hole and polar bridge formats, are described in Table 2 and Section 6.3.1.5 of WO 2019/104075 and subsections thereof, incorporated by reference herein. The Fc region can have altered effector function. The term “effector function” refers to an activity of an antibody molecule that is mediated by binding through a domain of the antibody other than the antigen binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Fc regions with altered effector function are described, for example, in Sections 6.3.1.1 through 6.3.1.5 of WO 2019/104075, incorporated by reference herein, and can include, for example altered binding to one or more Fc receptors such as FcRN, modified disulfide bond architecture, or altered glycosylation patterns as compared to a wild type Fc region.

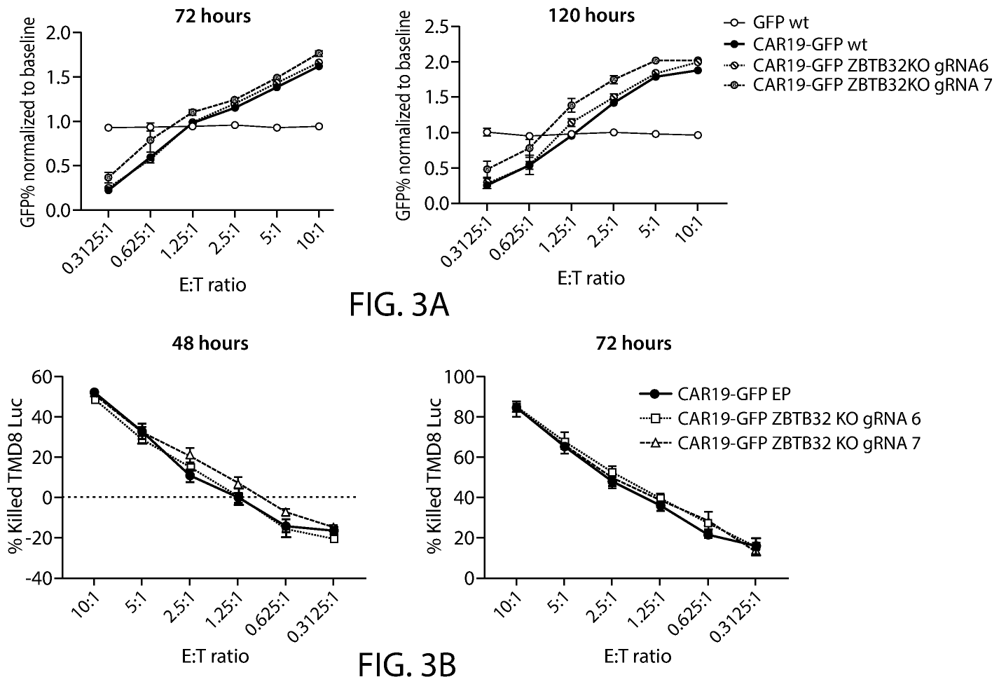

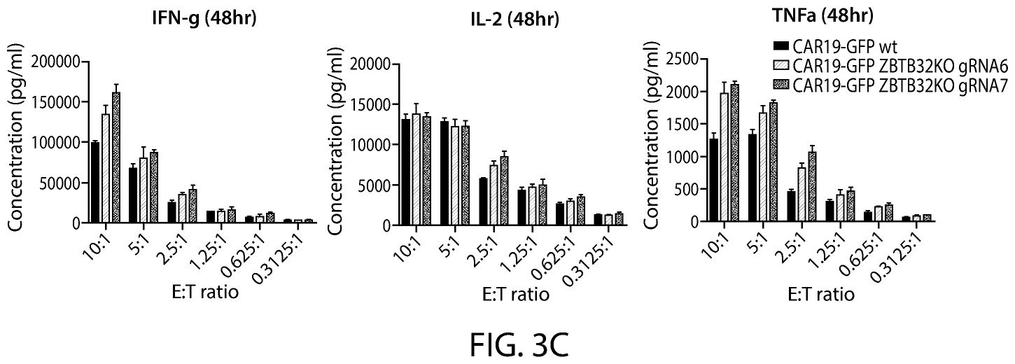

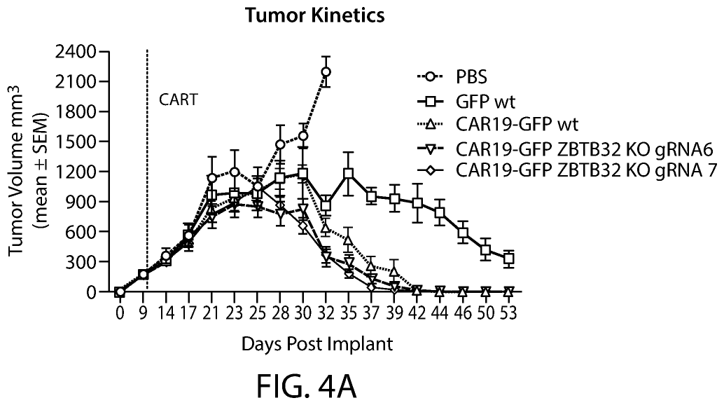

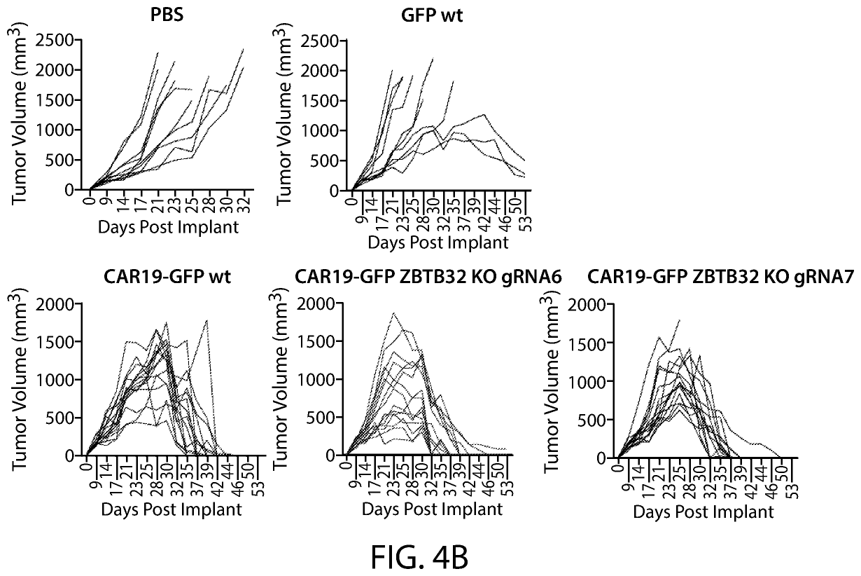

Additional MBM formats that can be used in combination with the ZBTB32 inhibitors of the disclosure are disclosed, inter alia, in FIG. 1 of Suurs et al., 2019, Pharmacology & Therapeutics 201:103-119, FIG. 2 of Labrijn et al., 2019, Nat Rev Drug Discov. 18(8):585-608; FIG. 4 of Krishnamurthy and Jimeno, 2018, Pharmacology and Therapeutics 185:122-134; FIG. 3 of Sedykh et al., 2018, Drug Design, Development and Therapy 12:195-208; FIG. 1 of Spiess et al., 2015, Molecular Immunology 67:95-106; FIG. 2 of Brinkmann & Kontermann, 2017, mAbs, 9:2, 182-212; FIG. 3 of Klein et al., 2016, mAbs, 8:6, 1010-1020;' and FIG. 2 of Klein et al., 2019, Methods 154:21-31, all of which figures and accompanying text are incorporated by reference herein.

A number of MBMs have been developed or are in development for treatment of a variety of cancers and can be used in combination of the ZBTB32 inhibitors of the disclosure. See, for example, Tables 1 and 2 of Labrijn et al., 2019, Nat Rev Drug Discov. 18(8):585-608; Tables 1 and 2 of Krishnamurthy and Jimeno, 2018, Pharmacology and Therapeutics 185:122-134; FIG. 3 of Suurs et al., 2019, Pharmacology & Therapeutics 201:103-119; Table 1 of Weidle et al., 2014, Seminars in Oncology 41:653-660, Table I of Sedykh et al., 2018, Drug Design, Development and Therapy 12:195-208; Table 1 of Spiess et al., 2015, Molecular Immunology 67:95-106; Table 1 of Dahlen et al., 2018, Therapeutic Advances in Vaccines and Immunotherapy 6(1) 3-17; and Table 3 of Brinkmann & Kontermann, 2017, mAbs, 9(2):182-212, all such tables and figures and accompanying text incorporated by reference herein. Exemplary MBMs that can be used in combination with the ZBTB32 inhibitors of the disclosure are set forth in the foregoing tables and figures as well as in [Table 25below.

TABLE 25
Exemplary MBMs

| Name(s) | | | |
|---|---|---|---|
| of MBM | ABDs | Proposed indication(s) | Reference/Clinical Trial No. |
| A-319 | Anti-CD3 × anti-CD19 | Hematologic malignancies (e.g., acute | NCT04056975 |
| | | lymphoblastic leukemia and B-cell acute | |
| | | lymphoblastic leukemia) | |
| A-337 | Anti-CD3 × anti-EpCAM | Solid malignancies (e.g., non-small-cell | ACTRN12617001181392 |
| | | lung cancer) | |
| ABBV-428 | Anti-CD40 × anti-MSLN | Solid malignancies | NCT02955251 |
| AFM11 | Anti-CD3 × anti-CD19 | Relapsed or refractory B-cell non-Hodgkin | Reusch et al., 2015, mAbs |
| | | lymphoma | 7: 584-604 |
| AFM13 | Anti-CD30 × anti-CD16A | Relapsed or refractory Hodgkin lymphoma | Malin et al., 2013, Clin Cancer |
| | | | Research 19: 2797-2803 |
| AK104 | Anti-PD-1 × anti-CTLA4 | Solid malignancies (e.g., gastric or | Ji et al., 2019, Annals of |
| | | gastroesophageal junction adenocarcinoma) | Oncology 30: v323-v324 |
| AMG160 | Anti-CD3 × anti-PSMA | Solid malignancies (e.g., prostate cancer) | NCT03792841 |
| AMG330 | Anti-CD3 × anti-CD33 | Relapsed or refractory acute myeloid leukemia | Aigner et al., 2013, Leukemia |
| | | | 27: 1107-1115 |
| AMG420 | Anti CD3 × anti-BCMA | Hematologic malignancies (e.g., multiple | Hipp et al., 2017, Leukemia |
| BI 836909 | | myeloma) | 31: 1743-1751 |
| AMG424 | Anti-CD3 × anti-CD38 | Hematologic malignancies (e.g., multiple | de Zafra et al., 2017, Blood |
| Xmab13551 | | myeloma) | 130: 500 and de Zafra et al., |
| | | | 2019, Clinical Cancer Research |
| | | | 25: 3921-3933 |
| AMG427 | Anti-CD3 × anti-FLT3 | Hematologic malignancies (e.g., acute | NCT03541369 |
| | | myeloid leukemia) | |
| AMG562 | Anti-CD3 × anti-CD19 | Lymphoma | Popplewell et al., 2019 |
| | | | Hematological Oncology |
| | | | 37: 566-567 |
| AMG596 | Anti-CD3 × anti-EGFRvIII | Solid malignancies (e.g., EGFRvIII+ | Rosenthal et al., 2019, |
| | | glioblastoma) | Neuro-oncology 21: 283 |
| AMG673 | Anti-CD3 × anti-CD33 | Hematologic malignancies (e.g., | Subklewe et al., 2019, |
| | | acute myeloid leukemia) | Blood 134: 833 |
| AMG701 | Anti-CD3 × anti BCMA | Hematologic malignancies (e.g., | Cho et al., 2018, Blood |
| | | multiple myeloma) | 132: 592 |
| AMG757 | Anti-CD3 × anti-DLL3 | Solid malignancies (e.g., small-cell | NCT03319940 |
| | | lung cancer) | |
| AMV-564 | Anti-CD3 × anti-CD33 | Hematologic malignancies (e.g., acute | Reusch et al., 2015, ASCO |
| | | myeloid leukemia and myelodysplastic | Annual Meeting Proceedings |
| | | syndrome) | |
| AMX-268 | Anti-CD3 × anti-EpCAM | Advanced solid tumors expressing EpCAM | Pearce et al., Poster 187 at |
| | | | 33rd Annual Meeting & Pre- |
| | | | Conference Programs of the |
| | | | Society for Immunotherapy of |
| | | | Cancer (SITC 2018), Washington, |
| | | | D.C., USA. 7-11 Nov. 2018. |
| APVO436 | Anti-CD3 × anti-CD123 | Hematologic malignancies (e.g., acute | Comeau et al., 2018, Cancer |
| | | myeloid leukemia and myelodysplastic | Research 78: 1786 |
| | | syndrome) | |
| ATTACK | Anti-EGFR × anti-CD3 | EGFR positive cancers | Harwood et al., 2017, |
| | | | OncoImmunology 7: 1, e1377874 |
| BI 836880 | Anti-ANG2 × anti-VEGF | Solid malignancies (e.g., non-small- | Hofmann et al., 2015, Poster |
| | | cell lung cancer) | presented at 8th Euro Global |
| | | | Summit on Cancer Therapy |
| Blinatumomab | Anti-CD3 × anti-CD19 | Hematologic malignancies (e.g., acute | Topp et al., 2012, Blood 120: |
| Blincyto | | lymphoblastic leukemia and B-cell acute | 5185-5187 |
| MT103 | | lymphoblastic leukemia) | |
| MEDI-538 | | | |
| AMG103 | | | |
| Bp-Bs | Anti-CD3 × anti-HER2 | HER2+ tumors | Liu et al., 2019, Mol. Ther. |
| | | | Oncolytics 14: 66-73 |
| Catumaxomab | Anti-CD3 × anti-EpCAM | Solid malignancies (e.g., malignant | Quintás-Cardama et al., 2010, |
| Removab | | ascites owing to epithelial carcinomas) | J Clin Oncol. 28: 884-92 |
| CC-93269 | Anti-CD3 × anti-BCMA | Hematologic malignancies (e.g., multiple | Seckinger et al., 2015 Blood |
| EM801 | | myeloma) | 126: 117 |
| Cibisatamab | Anti-CEA × anti-CD3 | Advanced CEA positive solid tumors | Tabernero et al., 2017, Journal |
| RG7802 | | | of Clinical Oncology, 35: 3002 |
| RO6958688 | | | |
| CEA-TCB | | | |
| Dilpacimab | Anti-DLL4 × anti-VEGF | Solid malignancies (e.g., colorectal | Li et al., 2018, Molecular |
| ABT-165 | | cancer) | cancer therapeutics 17: 1039-1050 |
| EMB01 | Anti-EGFR × anti-MET | Solid malignancies | NCT03797391 |
| Ertumaxomab | Anti-CD3 × anti-HER2 | HER-2 positive metastatic breast cancer | Kiewe et al., 2006, Clinical |
| | | | Cancer Research 12: 3085-3091 |
| ERY974 | Anti-CD3 × anti-FLTGPC33 | Solid malignancies (e.g., gastric cancer | Shiraiwa et al., 2019, Methods |
| | | and squamous cell oesophageal carcinoma) | 154: 10-20 |
| FBTA05 | Anti-CD3 × anti-CD20 | Relapsed or refractory B-cell lymphoma | Stanglmaier et al., 2008, Int. |
| | | after allogeneic SCT | J. Cancer 123: 1181-1189 |
| FS118 | Anti-PD-L1 × anti-LAG3 | Solid malignancies | Kraman et al., 2020 Clinical Cancer |
| | | | Research Online publication DOI: |
| | | | 10.1158/1078-0432.CCR-19-3548 |
| Gatipotuzumab- | Anti-TA-MUC1 × anti-CD3 | Solid tumors | https://www.glycotope.com/ |
| based T cell | | | biopharmaceuticals-pipeline/ |
| engager PM-CD3 | | | |
| GBR1302 | Anti-CD3 × anti-HER2 | Solid malignancies (e.g., HER2+ cancers) | Back et al., 2018, poster presented |
| | | | at the 54th Annual Meeting of the |
| | | | American Society of Clinical Oncology, |
| | | | Jun. 1-5, 2018 |
| | | | NCT02829372 |
| GBR1342 | Anti-CD3 × anti-CD38 | Hematologic malignancies (e.g., | Richter et al., 2018, J Clinical |
| | | multiple myeloma) | Oncol. 36: TPS3132 |
| GEM333 | Anti-CD3 × anti-CD33 | Hematologic malignancies (e.g., | NCT03516760 |
| | | acute myeloid leukemia) | |
| GEN3013 | Anti-CD3 × anti-CD20 | Hematologic malignancies (e.g., diffuse | Hiemstra et al., 2018, Blood |
| DuoBody- | | large B cell lymphoma, follicular | 132: 1664-1664 |
| CD3 × CD20 | | lymphoma and mantle cell lymphoma) | |
| GTB-3550 | Anti-CD16 × anti-CD33 | Hematologic malignancies (e.g., | NCT03214666 |
| OXS-3550 | | myelodysplastic syndrome, acute | |
| | | myeloid leukemia and systemic | |
| | | mastocytosis) | |
| IBI318 | Anti-PD-1 × anti- | Solid malignancies | NCT03875157 |
| | undisclosed TAA | | |
| INBRX-105 | Anti-PD-L1 × anti-4-1BB | Hematologic and solid malignancies | NCT03809624 |
| | | (e.g., lymphoma and solid tumors) | |
| JNJ-61186372 | Anti-EGFR × anti-MET | Solid malignancies (e.g., non-small- | Grugan et al., 2017, mAbs |
| | | cell lung cancer) | 9: 114-126 |
| JNJ-63709178 | Anti-CD3 × anti-CD123 | Hematologic malignancies (e.g., acute | Gaudet et al., 2016, Blood |
| | | myeloid leukemia) | 128: 2824 |
| JNJ-64007957 | Anti-CD3 × anti-BCMA | Hematologic malignancies (multiple | Girgis et al., 2016, Blood |
| | | myeloma) | 128:5668 |
| JNJ-64407564 | Anti-CD3 × anti-GPRC5D | Hematologic malignancies (e.g., multiple | Kodama et al., 2019, Molecular |
| | | myeloma) | Cancer Therapeutics 18: 1555-1564 |
| KN026 | Anti-HER2 × anti-HER2 | Solid malignancies (e.g., breast and | Wei et al., 2017, Oncotarget |
| | | gastric cancer) | 8: 51037-51049 |
| KN046 | Anti-PD-L1 × anti-CTLA4 | Solid and hematologic malignancies (e.g., | Coward et al., 2019, Journal of |
| | | triple-negative breast cancer, non- | Clinical Oncology 37: 2554 |
| | | small-cell lung cancer and lymphoma) | |
| LY3164530 | Anti-Her1 × anti-cMET | Advanced or metastatic cancer | Liu et al., 2016 Cancer Research |
| | | | 76: 873-873. |
| LY3415244 | Anti-PD-L1 × anti-TIM3 | Solid malignancies | Hellmann et al., 2019, American |
| | | | Society of Clinical Oncology (ASCO) |
| | | | Annual Meeting; Chicago, IL; May |
| | | | 31-Jun. 4, 2019: TPS2654 |
| M802 | Anti-CD3 × anti-HER2 | Solid malignancies (e.g., breast cancer | Labrijn refers to phase I trial but |
| | | and gastric cancer) | there's nothing out there (and they |
| | | | misspell it as YZYbio . . .) |
| MBS301 | Anti-HER2 × anti-HER2 | Solid malignancies (e.g., HER2+ solid | Sijia et al., 2018, mAbs 10: 864-875 |
| | | tumors) | |
| MCLA-145 | Anti-PD-L1 × anti-4-1BB | Solid malignancies | Mayes et al., 2019, Poster presented |
| | | | at the American Association for Cancer |
| | | | Research (AACR) 2019 Annual Meeting, |
| | | | Atlanta, GA, USA. |
| MCLA-158 | Anti-EGFR × anti-LGR5 | Solid malignancies (e.g., colorectal | Roovers et al., 2017, Poster presented |
| | | cancer) | at the American Association for Cancer |
| | | | Research (AACR) 2017 Annual Meeting, |
| | | | Washington, DC, USA. |
| MEDI5752 | Anti-PD-1 × anti-CTLA4 | Solid malignancies | Dovedi et al., 2018, Cancer Research |
| | | | 78: abstract 2776 |
| MGD006 | Anti-CD3 × anti-CD123 | Relapsed/refractory acute myeloid | Vey et al., 2017, Journal of Clinical |
| S80880 | | leukemia or intermediate 2/high | Oncology 35; abstr TPS7070 |
| | | risk myelodysplastic syndrome | |
| MGD007 | Anti-CD3 × anti-GPA33 | Relapsed refractory metastatic | Moore et al., 2014, Cancer Research |
| | | colorectal cancer | 74: 669 |
| MGD011 | Anti-CD19 × anti-CD3 | B-cell malignancies | Liu et al., 2017, Clinical Cancer. |
| | | | Research 23: 1506-1518 |
| MGD013 | Anti-PD-1 × anti-LAG3 | Solid and hematologic malignancies | LaMotte-Mohs et al., 2016, Cancer |
| | | | Res 76(14 Suppl): Abstract 3217 |
| MGD019 | Anti-PD-1 × anti-CTLA4 | Solid malignancies | Luke et al., 2019, Journal of |
| | | | Clinical Oncology 37: TPS2661 |
| MM111 | Anti-Her2 × anti-Her3 | Her-2 positive advanced solid tumors | Beeram et al., 2010 Cancer Research |
| | | | 70: 6-15 |
| MM141 | Anti-IGF1R × anti-Her3 | Advanced solid tumors | Friedrich et al., 2012, Mol Cancer |
| | | | Ther. 11: 2664-2673 |
| MOR209 | Anti-PSMA × anti-CD3 | Metastatic castrate resistant prostate | Hernandez-Hoyos et al., 2016, |
| ES414 | | cancer | Molecular cancer therapeutics |
| | | | 15: 2155-2165 |
| MOR209 | Anti-CD3 × anti-PSMA | Solid malignancies (e.g., prostate cancer) | Hernandez-Hoyos et al., 2016, |
| APVO414 | | | Mol. Cancer Ther. 15: 2155-2165 |
| ES414 | | | |
| Mosunetuzumab | Anti-CD3 × anti-CD20 | Hematologic malignancies (e.g., chronic | Budde et al., 2018, Blood 132: 399 |
| RG7828 | | lymphocytic leukemia, non-Hodgkin | |
| RO7030816 | | lymphoma and diffuse large B cell lymphoma) | |
| BTCT4465A | | | |
| MT110 | Anti-CD3 × anti-EpCAM | Advanced solid tumors expressing EpCAM | Fiedler et al., 2010, Journal of |
| AMG 110 | | | Clinical Oncology 28: 2573-2573 |
| MT111 | Anti-CD3 × anti-CEA | Advanced GI adenocarcinomas | El-Sahwi et al., 2010, Br J Cancer |
| AMG211 | | | 102: 134-143 |
| MEDI-565 | | | |
| MT112 | Anti-CD3 × anti-PSMA | Metastatic castrate resistant prostate | 25th EORTC-NCI-AACR., 2013, |
| BAY2010112 | | cancer | abstract C169 |
| NOV1501 | Anti-DLL4 × anti-VEGF | Solid malignancies | Lee et al., 2019, Journal of |
| ABL001 | | | Clinical Oncology 37: 3023 |
| TR009 | | | |
| OMP-305B83 | Anti-DLL4 × anti-VEGF | Advanced solid tumors | Jimeno et al., 2016, European |
| Navicixizumab | | | Journal of Cancer 69: S35 |
| Orlotamab | Anti-CD3 × anti-B7-H3 | Solid malignancies (non-small-cell lung | Tolcher et al., 2016, Journal of |
| MGD009 | | cancer and melanoma) | Clinical Oncology 37: TPS3105 |
| OXS-1550 | Anti-CD19 × anti-CD22 | Hematologic malignancies (e.g., B cell | Vallera et al., 2010, Molecular |
| DT2219ARL | | lymphoma and leukemia) | Cancer Therapeutics 9: 1872-1883 |
| Pasotuxizumab | Anti-CD3 × anti-PSMA | Solid malignancies (e.g., | Friedrich et al., 2012, Mol. |
| A212 | | prostate cancer) | Cancer Ther. 11: 2664-2673 |
| BAY2010112 | | | |
| PF-06671008 | Anti-CD3 × anti-P-cadherin | Solid malignancies (e.g., triple- | Root et al., 2016, Antibodies |
| | | negative breast cancer, non-small- | 5: 6 |
| | | cell lung cancer and colorectal cancer) | |
| PF-06863135 | Anti-CD3 × anti-BCMA | Hematologic malignancies (e.g., | Lesokhin et al., 2018, Blood |
| | | multiple myeloma) | 132: 3229 |
| Plamotamab | Anti-CD3 × anti-CD20 | Hematologic malignancies (e.g., | Chu et al., 2014, Blood 124: 3111 |
| XmAb13676 | | non-Hodgkin lymphoma and chronic | |
| | | lymphocytic leukemia) | |
| PRS-343 | Anti-HER2 × anti 4-1BB | HER2-positive solid tumors | Hinner et al., 2019, Clinical |
| | | | Cancer Research 25: 5878-5878 |
| REGN1979 | Anti-CD3 × anti-CD20 | Hematologic malignancies (e.g., | Smith et al., 2015, Scientific |
| | | follicular lymphoma, chronic | Reports 5: 17943 |
| | | lymphocytic leukemia and non- | |
| | | Hodgkin lymphoma) | |
| REGN4018 | Anti-CD3 × anti-MUC16 | Solid malignancies (e.g., | Crawford et al., 2019, Science |
| | | ovarian, fallopian tube or | Translational Medicine 11: |
| | | peritoneal cancers) | eaau7534. |
| REGN5458 | Anti-CD3 × anti-BCMA | Hematologic malignancies (e.g., | Dilillo et al., 2018, Blood |
| | | multiple myeloma) | 132: 1944 |
| RG6160, | Anti-CD3 × anti-FcRH5 | Hematologic malignancies (e.g., | NCT03275103 |
| RO7187797, | (CD307) | multiple myeloma) | |
| BFCR4350A | | | |
| RG6194 | Anti-CD3 × anti-HER2 | Solid malignancies (e.g., locally | NCT03448042 |
| BTRC4017A | | advanced or metastatic HER2- | |
| | | expressing cancers) | |
| RG7221 | Anti-angiopoietin 2 × | Advanced solid tumors | Kerbel et al., 2008, N Engl |
| RO5520985 | anti-VEGF-A | | J Med. 358: 2039-2049 |
| RG7597 | Anti-Her-1 × anti-Her-3 | Locally advanced or metastatic | Schaefer et al., 2011, Cancer |
| MEHD7945A | | epithelial tumors | Cell. 20: 472-486 |
| RO5541078 | | | |
| RO7082859 | Anti-CD3 × anti-CD20 | Hematologic malignancies (e.g., | Djebli et al., 2019, Blood |
| RG6026 | | non-Hodgkin lymphoma) | 134: 3799 |
| CD20-TCB | | | |
| RO7121661 | Anti-PD-1 × anti-TIM3 | Solid malignancies (e.g., non-small- | NCT03708328 |
| RG7769 | | cell lung cancer and melanoma) | |
| SAR440234 | Anti-CD3 × anti-CD123 | Hematologic malignancies (e.g., | NCT03594955 |
| | | acute myeloid leukemia, B-cell | |
| | | acute lymphoblastic leukemia and | |
| | | myelodysplastic syndrome) | |
| Tepoditamab | Anti-CD3 × anti-CLEC12A | Hematologic malignancies (e.g., | van Loo et al., 2019, Expert |
| MCLA-117 | | acute myeloid leukemia) | Opinion on Biological Therapy |
| | | | 19: 721-733 |
| TG-1801 | Anti-CD47 × anti-CD19 | Hematologic malignancies | Normant et al., 2019, Hematological |
| NI-1701 | | (e.g., B cell lymphoma) | Oncology 37: 322-323 |
| Tidutamab | Anti-CD3 × anti-SSTR2 | Solid malignancies (e.g., | NCT03411915 |
| XmAb18087 | | neuroendocrine and GIST) | |
| Vibecotamab | Anti-CD3 × anti-CD123 | Hematologic malignancies (e.g., | Chu et al., 2014, Blood1 24: 2316 |
| Xmab14045 | | acute myeloid leukemia, B-cell | |
| | | acute lymphoblastic leukemia and | |
| | | chronic myeloid leukemia) | |
| XmAb20717 | Anti-PD-1 × anti-CTLA4 | Solid malignancies | Hedvat et al., 2018, Cancer |
| | | | Research 78: abstract 2784 |
| XmAb23104 | Anti-PD-1 × anti-ICOS | Solid malignancies | Hedvat et al., 2018, Cancer |
| | | | Research 78: 2784 |
| Zenocutuzumab | Anti-HER2 × anti-HER3 | Solid malignancies (e.g., | de Vries Schultink et al., 2018 |
| MCLA-128 | | breast cancer) | Investigational New Drugs |
| PB4188 | | | 36: 1006-1015 |
| ZW25 | Anti-HER2 × anti-HER2 | Solid malignancies (e.g., | Weisser et al., 2017, Cancer. |
| | | HER2+ solid tumors) | Research 77: 31 |
| ZW49 | Anti-HER2 × anti-HER2 | Solid malignancies (e.g., | Hamblett et al., 2018, Cancer. |
| | ADC | HER2+ solid tumors | Research 8: Abstract 3914 |

Accordingly, the molecules of the disclosure can be administered in combination with any MBM described [Table 25], for example to treat a cancer indicated for that MBM in [Table 25] above.

In other embodiments, the MBM described in [Table 25] binds to BCMA, for example the AMG701, AMG701, CC-93269, JNJ-64007957, PF-06863135, or REGN5458. The molecules of the disclosure can be used in combination with any of the foregoing BCMA-targeting MBMs to treat hematologic cancers such as multiple myeloma, Hodgkin's and non-Hodgkin's lymphoma, various leukemias, and glioblastoma.

In other embodiments, the MBM described in [Table 25] binds to CD19, for example the MBM is A-319, AMG562, Blinatumomab, MGD011, or OXS-1550. The molecules of the disclosure can be used in combination with any of the foregoing CD19-targeting MBMs to treat hematologic cancers such as non-Hodgkin's lymphoma (B-NHL), chronic lymphocytic leukemia, and acute lymphoblastic leukemia.

### Other Therapeutic Agents

In another embodiment, the ZBTB32 inhibitor, or a pharmaceutically acceptable salt, hydrate, solvate, prodrug, stereoisomer, or tautomer thereof, of the present disclosure are used in combination with one or more of the therapeutic agents listed in Table 26 or listed in the patent and patent applications cited in Table 26, to treat cancer. Each publication listed in Table 26 is herein incorporated by reference in its entirety, including all structural formulae therein.

TABLE 26
Other Exemplary Therapeutic Agents

| Second | | | Patents/Patent |
|---|---|---|---|
| agent | Generic Name | | Application |
| No. | Tradename | Compound Structure | Publications |
| A1 | Sotrastaurin | 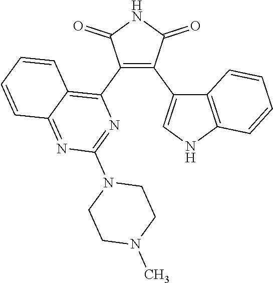 [SMILES: CN1CCN(c2nc(C3=C(c4c[nH]c5ccccc45)C(=O)NC3=O)c3ccccc3n2)CC1] | EP 1682103 US 2007/142401 WO 2005/039549 |
| A2 | Nilotinib HCl monohydrate TASIGNA ® | 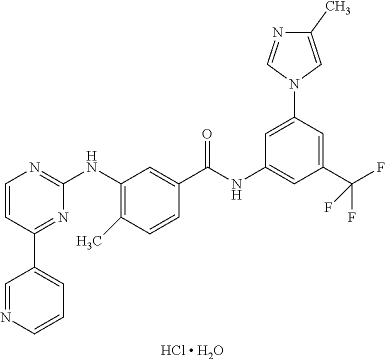 [SMILES: Cc1cn(-c2cc(NC(=O)c3ccc(C)c(Nc4nccc(-c5cccnc5)n4)c3)cc(C(F)(F)F)c2)cn1 ; "HCl · H2O"] | WO 2004/005281 U.S. Pat. No. 7,169,791 |
| A3 | | 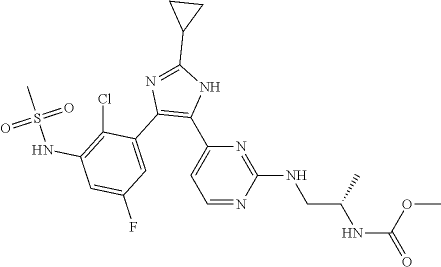 [SMILES: COC(=O)N[C@@H](C)CNc1nccc(-c2[nH]c(C3CC3)nc2-c2cc(F)cc(NS(C)(=O)=O)c2Cl)n1] | WO2011/023773 |
| A4 | | 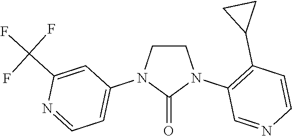 [SMILES: O=C1N(c2ccnc(C(F)(F)F)c2)CCN1c1cnccc1C1CC1] | WO2012/149413 |
| A6 | | 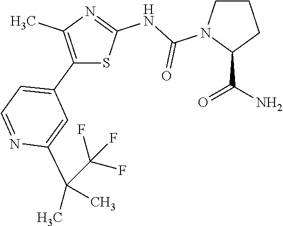 [SMILES: Cc1nc(NC(=O)N2CCC[C@H]2C(N)=O)sc1-c1ccnc(C(C)(C)C(F)(F)F)c1] | WO 2010/029082 |
| A7 | | 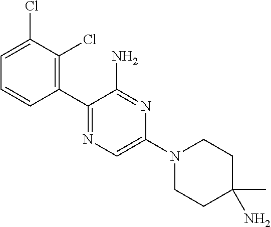 [SMILES: CC1(N)CCN(c2cnc(-c3cccc(Cl)c3Cl)c(N)n2)CC1] | WO2015/107493 |
| A8 | | | WO2015/107495 |
| A9 | | 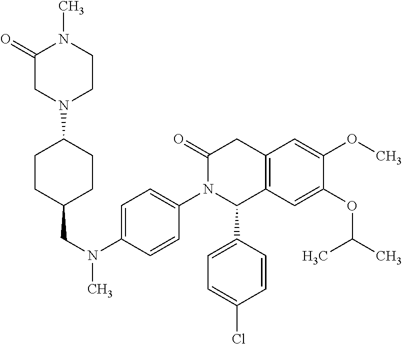 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2] | WO 2011/076786 |
| A10 | Deferasirox EXJADE ® | 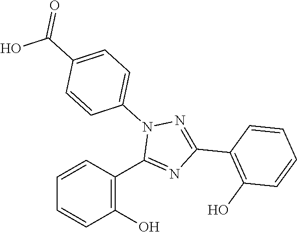 [SMILES: O=C(O)c1ccc(-n2nc(-c3ccccc3O)nc2-c2ccccc2O)cc1] | WO 1997/049395 |
| A11 | Letrozole FEMARA ® | 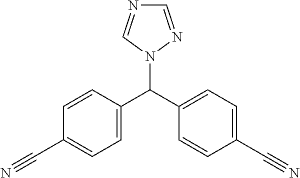 [SMILES: N#Cc1ccc(C(c2ccc(C#N)cc2)n2cncn2)cc1] | U.S. Pat. No. 4,978,672 |
| A12 | | 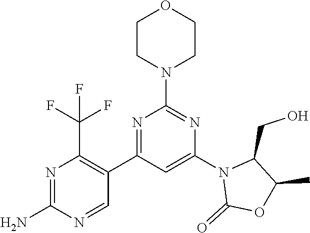 [SMILES: C[C@H]1OC(=O)N(c2cc(-c3cnc(N)nc3C(F)(F)F)nc(N3CCOCC3)n2)[C@H]1CO] | WO 2013/124826 US 2013/0225574 |
| A13 | | 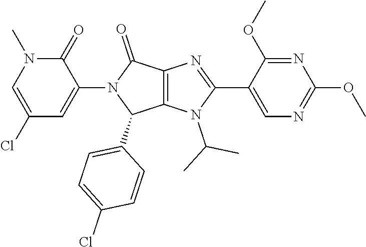 [SMILES: COc1ncc(-c2nc3c(n2C(C)C)[C@H](c2ccc(Cl)cc2)N(c2cc(Cl)cn(C)c2=O)C3=O)c(OC)n1] | WO 2013/111105 |
| A14 | | 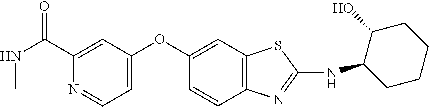 [SMILES: CNC(=O)c1cc(Oc2ccc3nc(N[C@@H]4CCCC[C@H]4O)sc3c2)ccn1] | WO2007/121484 |
| A15 | Imatinib mesylate GLEEVEC ® | 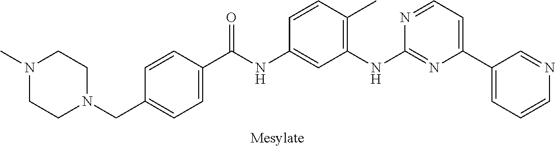 [SMILES: Cc1ccc(NC(=O)c2ccc(CN3CCN(C)CC3)cc2)cc1Nc1nccc(-c2cccnc2)n1] | WO 1999/003854 |
| A16 | Capmatinib | 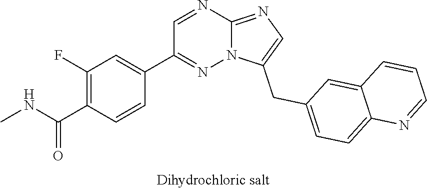 [SMILES: CNC(=O)c1ccc(-c2cnc3ncc(Cc4ccc5ncccc5c4)n3n2)cc1F] | EP 2099447 U.S. Pat. No. 7,767,675 U.S. Pat. No. 8,420,645 |
| A17 | Ruxolitinib Phosphate JAKAFI ® | 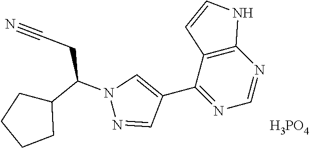 | WO 2007/070514; EP 2474545 U.S. Pat. No. 7,598,257; WO 2014/018632 |
| A18 | Panobinostat | 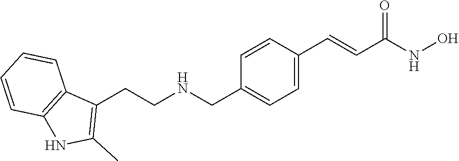 [SMILES: Cc1[nH]c2ccccc2c1CCNCc1ccc(/C=C/C(=O)NO)cc1] | WO 2014/072493 WO 2002/022577 EP 1870399 |
| A20 | | 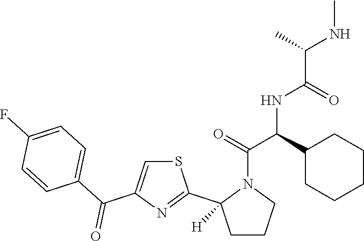 [SMILES: CN[C@@H](C)C(=O)N[C@H](C(=O)N1CCC[C@H]1c1nc(C(=O)c2ccc(F)cc2)cs1)C1CCCCC1] | WO 2008/016893 EP 2051990 U.S. Pat. No. 8,552,003 |
| A21 | | 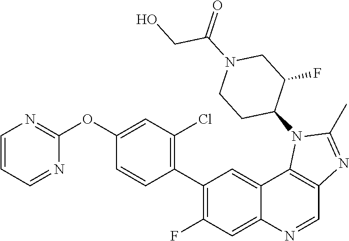 [SMILES: Cc1nc2cnc3cc(F)c(-c4ccc(Oc5ncccn5)cc4Cl)cc3c2n1[C@H]1CCN(C(=O)CO)C[C@@H]1F] | WO2015/022662 |
| A22 | ceritinib ZYKADIA ™ | 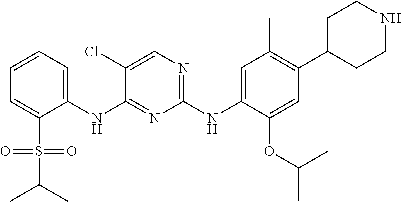 [SMILES: Cc1cc(Nc2ncc(Cl)c(Nc3ccccc3S(=O)(=O)C(C)C)n2)c(OC(C)C)cc1C1CCNCC1] | WO 2008/073687 U.S. Pat. No. 8,039,479 |
| A23 | Ribociclib KISQALI ® | 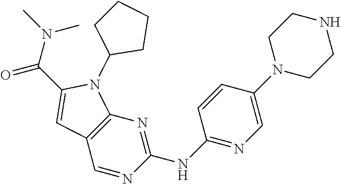 [SMILES: CN(C)C(=O)c1cc2cnc(Nc3ccc(N4CCNCC4)cn3)nc2n1C1CCCC1] | U.S. Pat. No. 8,415,355 U.S. Pat. No. 8,685,980 |
| A24 | | 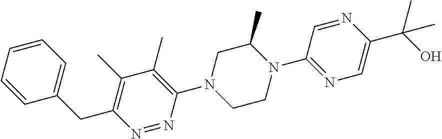 [SMILES: Cc1c(Cc2ccccc2)nnc(N2CCN(c3cnc(C(C)(C)O)cn3)[C@H](C)C2)c1C] | WO 2010/007120 |
| A26 | | 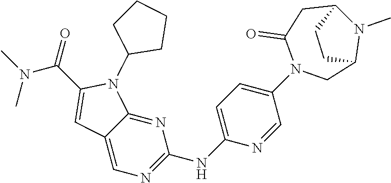 | WO 2011/101409 |
| A27 | | Human monoclonal antibody to HER3 | WO 2012/022814 |
| | | | EP 2606070 |
| | | | U.S. Pat. No. 8,735,551 |
| A28 | | Antibody Drug Conjugate (ADC) | WO 2014/160160 |
| A29 | | Monoclonal antibody or Fab to M-CSF | WO 2004/045532 |
| A30 | Midostaurin | 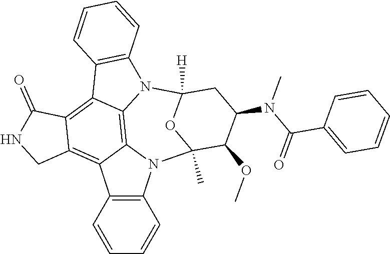 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4] | WO 2003/037347 EP 1441737 US 2012/252785 |
| A31 | Everolimus AFINITOR ® | 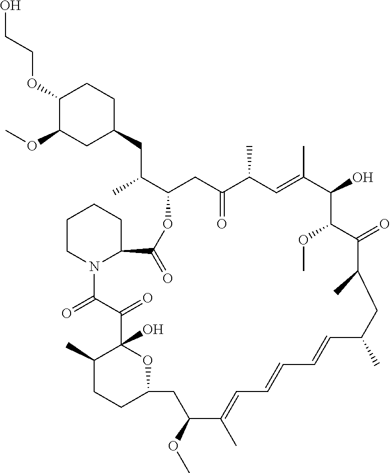 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] | WO 1994/009010 WO 2014/085318 |
| A32 | | 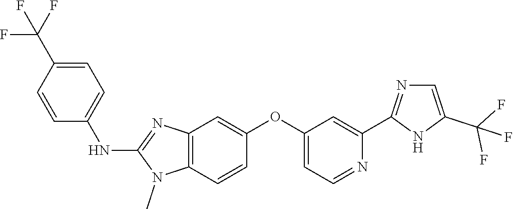 [SMILES: Cn1c(Nc2ccc(C(F)(F)F)cc2)nc2cc(Oc3ccnc(-c4ncc(C(F)(F)F)[nH]4)c3)ccc21] | WO 2007/030377 U.S. Pat. No. 7,482,367 |
| A34 | | 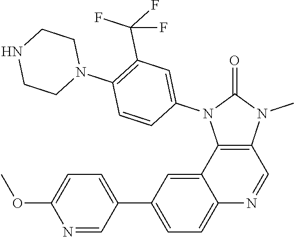 | WO 2006/122806 |
| A35 | | 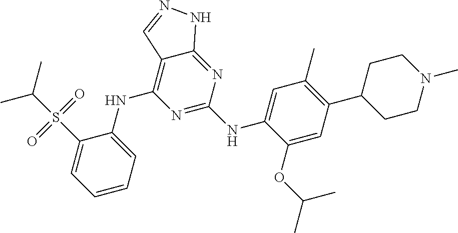 [SMILES: Cc1cc(Nc2nc(Nc3ccccc3S(=O)(=O)C(C)C)c3cn[nH]c3n2)c(OC(C)C)cc1C1CCN(C)CC1] | WO 2008/073687 U.S. Pat. No. 8,372,858 |
| A36 | Valspodar AMDRAY ™ | 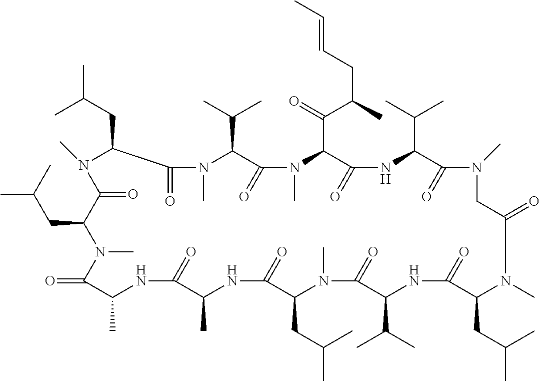 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | EP 296122 |
| A37 | Vatalanib succinate | 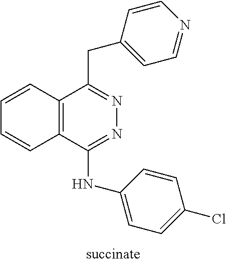 [SMILES: Clc1ccc(Nc2nnc(Cc3ccncc3)c3ccccc23)cc1] | WO 98/35958 |
| A38 | | 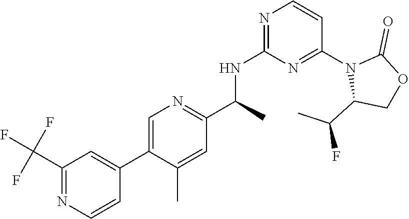 [SMILES: Cc1cc([C@H](C)Nc2nccc(N3C(=O)OC[C@@H]3[C@H](C)F)n2)ncc1-c1ccnc(C(F)(F)F)c1] | WO2014/141104 |
| A39 | Asciminib | 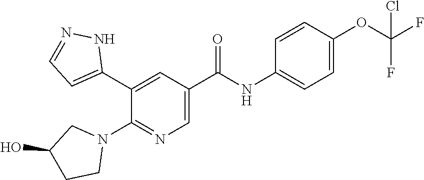 [SMILES: O=C(Nc1ccc(OC(F)(F)Cl)cc1)c1cnc(N2CC[C@@H](O)C2)c(-c2ccn[nH]2)c1] | WO2013/171639 WO2013/171640 WO2013/171641 WO2013/171642 |
| A42 | | 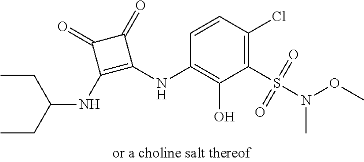 [SMILES: CCC(CC)Nc1c(Nc2ccc(Cl)c(S(=O)(=O)N(C)OC)c2O)c(=O)c1=O] | WO2010/015613 WO2013030803 U.S. Pat. No. 7,989,497, |
| A43 | | | WO 2017/025918 |
| | | | WO2011/121418 |
| | | | U.S. Pat. No. 8,796,284 |
| A44 | | 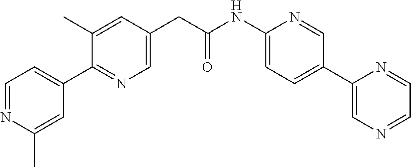 [SMILES: Cc1cc(-c2ncc(CC(=O)Nc3ccc(-c4cnccn4)cn3)cc2C)ccn1] | WO2010/101849 |
| A45 | | 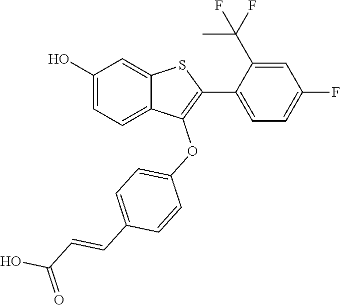 [SMILES: CC(F)(F)c1cc(F)ccc1-c1sc2cc(O)ccc2c1Oc1ccc(/C=C/C(=O)O)cc1] | WO2014/130310 |
| A46 | trametinib | 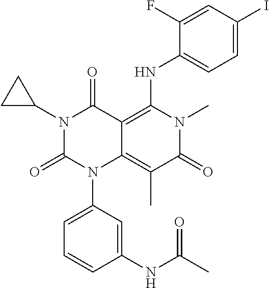 [SMILES: CC(=O)Nc1cccc(-n2c(=O)n(C3CC3)c(=O)c3c(Nc4ccc(I)cc4F)n(C)c(=O)c(C)c32)c1] | WO2005/121142 U.S. Pat. No. 7,378,423 |
| A47 | dabrafenib | 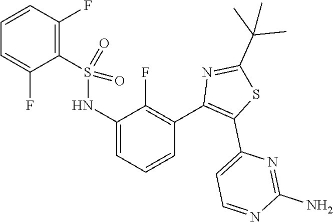 [SMILES: CC(C)(C)c1nc(-c2cccc(NS(=O)(=O)c3c(F)cccc3F)c2F)c(-c2ccnc(N)n2)s1] | WO 2009/137391 U.S. Pat. No. 7,994,185 |
| A49 | octreotide | 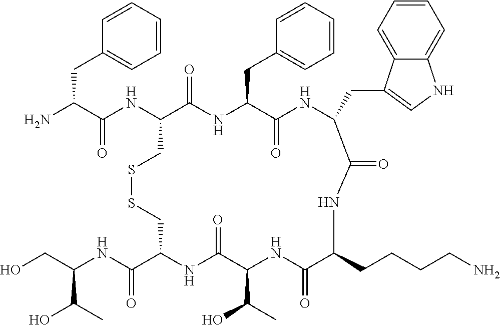 [SMILES: CC(O)[C@@H](CO)NC(=O)[C@@H]1CSSC[C@H](NC(=O)[C@H](N)Cc2ccccc2)C(=O)N[C@@H](Cc2ccccc2)C(=O)N[C@H](Cc2c[nH]c3ccccc23)C(=O)N[C@@H](CCCCN)C(=O)N[C@@H]([C@@H](C)O)C(=O)N1] | U.S. Pat. No. 4,395,403 EP 0 029 579 |
| A50 | | 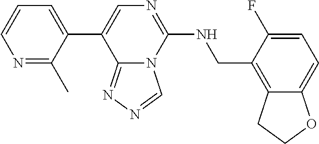 [SMILES: Cc1ncccc1-c1cnc(NCc2c(F)ccc3c2CCO3)n2cnnc12] | WO 2016/103155 U.S. Pat. No. 9,580,437 EP 3237418 |
| A51 | | 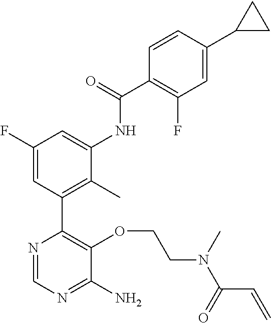 [SMILES: C=CC(=O)N(C)CCOc1c(N)ncnc1-c1cc(F)cc(NC(=O)c2ccc(C3CC3)cc2F)c1C] | U.S. Pat. No. 9,512,084 WO/2015/079417 |
| A52 | | 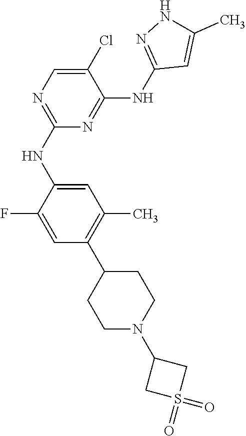 [SMILES: Cc1cc(Nc2nc(Nc3cc(C)c(C4CCN(C5CS(=O)(=O)C5)CC4)cc3F)ncc2Cl)n[nH]1] | WO 2010/002655 U.S. Pat. No. 8,519,129 |
| A53 | | 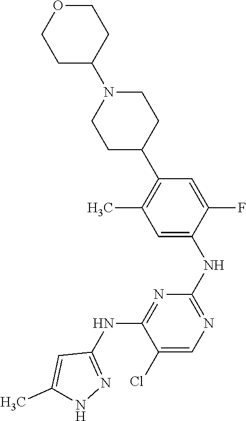 [SMILES: Cc1cc(Nc2nc(Nc3cc(C)c(C4CCN(C5CCOCC5)CC4)cc3F)ncc2Cl)n[nH]1] | WO 2010/002655 U.S. Pat. No. 8,519,129 |
| A54 | | 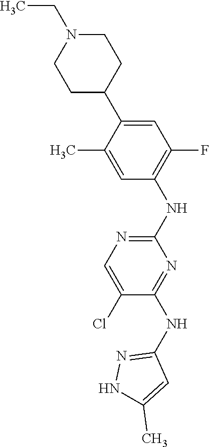 [SMILES: CCN1CCC(c2cc(F)c(Nc3ncc(Cl)c(Nc4cc(C)[nH]n4)n3)cc2C)CC1] | WO 2010/002655 |

### Pharmaceutical Compositions for ZBTB32 Inhibitors

In another aspect, the present disclosure provides compositions, e.g., pharmaceutically acceptable compositions, which includes a ZBTB32 inhibitor (e.g., a ZBTB32 inhibitor described herein), alone or in combination with a second therapeutic agent (e.g., a therapeutic agent described herein), formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this disclosure may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In certain embodiments, the mode of administration is parenteral (e.g., intravenous, subcutaneous, intraperitoneal, or intramuscular). In an embodiment, the composition is administered by intravenous infusion or injection. In another embodiment, the composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, a ZBTB32 inhibitor (e.g., a ZBTB32 inhibitor described herein), alone or in combination with a second therapeutic agent (e.g., a therapeutic agent described herein), can be formulated into a formulation (e.g., a dose formulation or dosage form) suitable for administration (e.g., intravenous administration) to a subject as described herein.

The therapeutic agents, e.g., inhibitors, antagonist or binding agents, can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic agent or compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the disclosure by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

The pharmaceutical compositions of the disclosure may include a “therapeutically effective amount” or a “prophylactically effective amount” of a compound of the disclosure. A “therapeutically effective amount” refers to an amount effective, at dosages and for periods of time necessary, to achieve the desired therapeutic result. A therapeutically effective amount of a compound may vary according to factors such as the disease state, age, sex, and weight of the individual, and the ability of the compound to elicit a desired response in the individual. A therapeutically effective amount is also one in which any toxic or detrimental effects of the compound is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### Biomarkers

In certain embodiments, any of the methods disclosed herein further includes evaluating or monitoring the effectiveness of a therapy (e.g., a monotherapy or a combination therapy) described herein, in a subject (e.g., a subject having a cancer, e.g., a cancer described herein). The method includes acquiring a value of effectiveness to the therapy, wherein said value is indicative of the effectiveness of the therapy.

In embodiments, the value of effectiveness to the therapy comprises a measure of one, two, three, four, five, six, seven, eight, nine or more (e.g., all) of the following:
(i) a parameter of a tumor infiltrating lymphocyte (TIL) phenotype;
(ii) a parameter of a myeloid cell population;
(iii) a parameter of a surface expression marker;
(iv) a parameter of a biomarker of an immunologic response;
(v) a parameter of a systemic cytokine modulation;
(vi) a parameter of circulating free DNA (cfDNA);
(vii) a parameter of systemic immune modulation;
(viii) a parameter of microbiome;
(ix) a parameter of a marker of activation in a circulating immune cell; or
(x) a parameter of a circulating cytokine.

In some embodiments, the parameter of a TIL phenotype comprises the level or activity of one, two, three, four or more (e.g., all) of Hematoxylin and eosin (H&E) staining for TIL counts, CD8, FOXP3, CD4, or CD3, in the subject, e.g., in a sample from the subject (e.g., a tumor sample).

In some embodiments, the parameter of a myeloid cell population comprises the level or activity of one or both of CD68 or CD163, in the subject, e.g., in a sample from the subject (e.g., a tumor sample).

In some embodiments, the parameter of a surface expression marker comprises the level or activity of one or more (e.g., two, three, four, or all) of PD-1, PD-L1, LAG-3, TIM-3, or GITR, in the subject, e.g., in a sample from the subject (e.g., a tumor sample). In certain embodiments, the level of PD-1, PD-L1, LAG-3, TIM-3, or GITR is determined by immunohistochemistry (IHC).

In some embodiments, the parameter of a biomarker of an immunologic response comprises the level or sequence of one or more nucleic acid-based markers, in the subject, e.g., in a sample from the subject (e.g., a tumor sample).

In some embodiments, the parameter of systemic cytokine modulation comprises the level or activity of one, two, three, four, five, six, seven, eight, or more (e.g., all) of IL-18, IFN-γ, ITAC (CXCL11), IL-6, IL-1β, IL-4, IL-17, IL-15, or TGF-beta, in the subject, e.g., in a sample from the subject (e.g., a blood sample, e.g., a plasma sample).

In some embodiments, the parameter of cfDNA comprises the sequence or level of one or more circulating tumor DNA (cfDNA) molecules (e.g., tumor mutation burden), in the subject, e.g., in a sample from the subject (e.g., a blood sample, e.g., a plasma sample).

In some embodiments, the parameter of systemic immune-modulation comprises phenotypic characterization of an activated immune cell, e.g., a CD3-expressing cell, a CD8-expressing cell, or both, in the subject, e.g., in a sample from the subject (e.g., a blood sample, e.g., a PBMC sample).

In some embodiments, the parameter of microbiome comprises the sequence or expression level of one or more genes in the microbiome, in the subject, e.g., in a sample from the subject (e.g., a stool sample).

In some embodiments, the parameter of a marker of activation in a circulating immune cell comprises the level or activity of one, two, three, four, five or more (e.g., all) of circulating CD8+, HLA-DR+Ki67+, T cells, IFN-γ, IL-18, or CXCL 11 (IFN-γ induced CCK) expressing cells, in a sample (e.g., a blood sample, e.g., a plasma sample).

In some embodiments, the parameter of a circulating cytokine comprises the level or activity of IL-6, in the subject, e.g., in a sample from the subject (e.g., a blood sample, e.g., a plasma sample).

In some embodiments of any of the methods disclosed herein, the therapy comprises a combination described herein (e.g., a combination comprising a therapeutically effective amount of a PD-1 inhibitor described herein).

In some embodiments of any of the methods disclosed herein, the measure of one or more of (i)-(x) is obtained from a sample acquired from the subject. In some embodiments, the sample is chosen from a tumor sample, a blood sample (e.g., a plasma sample or a PBMC sample), or a stool sample.

In some embodiments of any of the methods disclosed herein, the subject is evaluated prior to receiving, during, or after receiving, the therapy.

In some embodiments of any of the methods disclosed herein, the measure of one or more of (i)-(x) evaluates a profile for one or more of gene expression, flow cytometry or protein expression.

In some embodiments of any of the methods disclosed herein, the presence of an increased level or activity of one, two, three, four, five, or more (e.g., all) of circulating CD8+, HLA-DR+Ki67+, T cells, IFN-γ, IL-18, or CXCL11 (IFN-γ induced CCK) expressing cells, and/or the presence of an decreased level or activity of IL-6, in the subject or sample, is a positive predictor of the effectiveness of the therapy.

Alternatively, or in combination with the methods disclosed herein, responsive to said value, performing one, two, three, four or more (e.g., all) of:
(i) administering to the subject the therapy;
(ii) administered an altered dosing of the therapy;
(iii) altering the schedule or time course of the therapy;
(iv) administering to the subject an additional agent (e.g., a therapeutic agent described herein) in combination with the therapy; or
(v) administering to the subject an alternative therapy.

### Kits for ZBTB32 Inhibitors

A combination of therapeutic agents disclosed herein can be provided in a kit. The therapeutic agents are generally provided in a vial or a container. As appropriate, the therapeutic agents can be in liquid or dried (e.g., lyophilized) form. The kits can comprise two or more (e.g., three, four, five, or all) of the therapeutic agents of a combination disclosed herein. In some embodiments, the kit further contains a pharmaceutically acceptable diluent. The therapeutic agents can be provided in the kit in the same or separate formulations (e.g., as mixtures or in separate containers). The kits can contain aliquots of the therapeutic agents that provide for one or more doses. If aliquots for multiple administrations are provided, the doses can be uniform or varied. Varied dosing regimens can be escalating or decreasing, as appropriate. The dosages of the therapeutic agents in the combination can be independently uniform or varying. The kit can include one or more other elements including: instructions for use; other reagents, e.g., a label, or an agent useful for chelating, or otherwise coupling, a therapeutic agent to a label or therapeutic agent, or a radioprotective composition; devices or other materials for preparing the antibody for administration; pharmaceutically acceptable carriers; and devices or other materials for administration to a subject.

### IKZF2 Inhibitors

In some embodiments, the ZBTB32 inhibitor, or a pharmaceutically acceptable salt, hydrate, solvate, prodrug, stereoisomer, or tautomer thereof, can be used in combination with an inhibitor of IKAROS Family Zinc Finger 2 (IKZF2), for making a CAR-expressing cell, e.g., an CAR-expressing immune effector cell, or for treating a disease, e.g., cancer.

In some embodiments, the IKZF2 inhibitor comprises a compound of Formula (I′)

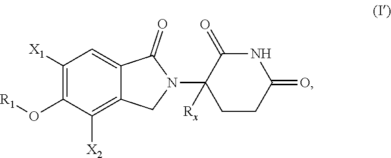

wherein:
X₁ and X₂ are each independently H, (C₁-C₄)alkyl, (C₁-C₆)alkoxy, (C₁-C₄)haloalkyl, (C₁-C₆)haloalkoxy, (C₃-C₇)cycloalkyl, halogen, —CN, —OH, or —NH₂;
Rₓ is or D;
R₁ is

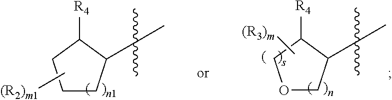

each R₂ is independently at each occurrence (C₁-C₆)alkyl, (C₁-C₆)alkoxy, (C₁-C₆)haloalkyl, (C₁-C₆)haloalkoxy, halogen, —CN, —OH, or —NH₂; or
two R₂ together with the carbon atoms to which they are attached form a (C₃-C₇)cycloalkyl or a 4- to 7-membered heterocycloalkyl ring comprising 1-3 heteroatoms selected from O, N, and S; or two R₂ together when on adjacent carbon atoms form a phenyl or a 5- or 6-membered heteroaryl ring comprising 1-3 heteroatoms selected from O, N, and S; or
R₂ and R₆ together with the carbon and nitrogen atoms to which they are attached form a 4- to 6-membered heterocycloalkyl ring optionally comprising 1-2 additional heteroatoms selected from 0, N, and S, and optionally substituted with one to four substituents each independently selected from (C1-C₆)alkyl, (C1-C₆)haloalkyl, halogen, —OH, —CN, and —NH₂;
each R₃ is (C₁-C₆)alkyl, (C₁-C₆)alkoxy, (C₁-C₆)haloalkyl, (C₁-C₆)haloalkoxy, halogen, —OH, or —NH₂;
R₄ is —OR₅ or —NR₆R₆;
R₅ is H, (C₁-C₆)alkyl, (C₁-C₆)haloalkyl, (C₃-C₇)cycloalkyl, 5- or 6-membered heterocycloalkyl comprising 1-3 heteroatoms selected from O, N, and S, (C₆-C₁₀)aryl, or 5- or 6-membered heteroaryl comprising 1-3 heteroatoms selected from O, N, and S, wherein the alkyl is optionally substituted with one to three substituents independently selected from (C₆-C₁₀)aryl and 5- or 6-membered heteroaryl comprising 1-3 heteroatoms selected from O, N, and S;
R₆ and R₆, are each independently H, (C1-C₆)alkyl, (C1-C₆)haloalkyl, (C₂-C₆)hydroxyalkyl, (C₃-C₇)cycloalkyl, 5- or 6-membered heterocycloalkyl comprising 1-3 heteroatoms selected from O, N, and S, (C₆-C₁₀)aryl, or 5- or 6-membered heteroaryl comprising 1-3 heteroatoms selected from O, N, and S, wherein the alkyl is optionally substituted with one to three R₇ and wherein the cycloalkyl, heterocycloalkyl, aryl, and heteroaryl are optionally substituted with one to four R₁₂; or
R₆ and R₆, together with the nitrogen atom to which they are attached form a 4- to 8-membered heterocycloalkyl ring optionally comprising 1-2 additional heteroatoms selected from O, N, and S, and optionally substituted with one to four R₈; or R₂ and R₆ together with the carbon and nitrogen atoms to which they are attached form a 4- to 6-membered heterocycloalkyl ring optionally comprising 1-2 additional heteroatoms selected from 0, N, and S, and optionally substituted with one to four substituents each independently selected from (C₁-C₆)alkyl, (C₁-C₆)haloalkyl, halogen, —OH, —CN, and —NH₂;
each R₇ is (C₃-C₇)cycloalkyl, 4- to 7-membered heterocycloalkyl ring comprising 1-3 heteroatoms selected from O, N, and S, (C₆-C₁₀)aryl, or 5- or 6-membered heteroaryl comprising 1-3 heteroatoms selected from O, N, and S, wherein the cycloalkyl, heterocycloalkyl, aryl, and heteroaryl are optionally substituted with one to four R₉;
each R₈ is independently at each occurrence halogen, (C1-C₆)alkyl, (C1-C₆)alkoxy, (C1-C₆)haloalkyl, (C1-C₆)haloalkoxy, —CN, —OH, —NR₁₃R₁₄, —NH₂, —O(C₃-C₇)cycloalkyl, —O-4- to 7-membered heterocycloalkyl ring comprising 1-3 heteroatoms selected from O, N, and S, —O(C₆-C₁₀)aryl, or —O-5- or 6-membered heteroaryl comprising 1-3 heteroatoms selected from O, N, and S, wherein the alkoxy is optionally substituted with one to three R₁₀ and the cycloalkyl, heterocycloalkyl, aryl, and heteroaryl are optionally substituted with one to three R₁₁; or
two R₈ together with the atoms to which they are attached form a (C₄-C₇)cycloalkyl or a 4- to 7-membered heterocycloalkyl ring comprising 1-2 heteroatoms selected from O, N, and S optionally substituted with two R₁₅; or two R₈ when on adjacent atoms together with the atoms to which they are attached form a (C₆-C₁₀)aryl, or 5- or 6-membered heteroaryl comprising 1-3 heteroatoms selected from O, N, and S; or two R₈ together with the same atom to which they are attached form a=(O);
each R₉ is independently at each occurrence (C₁-C₆)alkyl, (C₁-C₆)haloalkyl, (C₁-C₆)alkoxy, (C₁-C₆)haloalkoxy, halogen, (C₃-C₆)cycloalkyl, —OH, —CN, —NH₂, or —NR₁₃R₁₄; or
two R₉ together with the atoms to which they are attached form a (C₄-C₇)cycloalkyl or a 5- to 7-membered heterocycloalkyl ring comprising 1-2 heteroatoms selected from O, N, and S optionally substituted with one or more substituents each independently selected from (C₁-C₆)alkyl, (C₁-C₆)haloalkyl, halogen, —OH, —CN, and —NH₂; or two R₉ when on adjacent atoms together with the atoms to which they are attached form a (C₆-C₁₀)aryl, or 5- or 6-membered heteroaryl comprising 1-3 heteroatoms selected from O, N, and S;
each R₁₀ is independently at each occurrence selected from (C₃-C₇)cycloalkyl, 4- to 7-membered heterocycloalkyl ring comprising 1-3 heteroatoms selected from O, N, and S, (C₆-C₁₀)aryl, and 5- or 6-membered heteroaryl comprising 1-3 heteroatoms selected from O, N, and S;
each R₁₁ is independently at each occurrence selected from (C₁-C₆)alkyl, (C₁-C₆)haloalkyl, (C₁-C₆)alkoxy, (C₁-C₆)haloalkoxy, halogen, —OH, —CN, and —NH₂;
each R₁₂ is independently at each occurrence (C₁-C₆)alkyl, (C₁-C₆)haloalkyl, (C₁-C₆)alkoxy, (C₁-C₆)haloalkoxy, halogen, —OH, —CN, or —NH₂;
two R₁₂ together with the atoms to which they are attached form a (C₄-C₇)cycloalkyl or a 4- to 7-membered heterocycloalkyl ring comprising 1-2 heteroatoms selected from O, N, and S;
R₁₃ and R₁₄ are each independently selected from (C₁-C₆)alkyl, (C₁-C₆)haloalkyl, (C₃-C₇)cycloalkyl, 4- to 7-membered heterocycloalkyl ring comprising 1-3 heteroatoms selected from O, N, and S, (C₆-C₁₀)aryl, and 5- or 6-membered heteroaryl comprising 1-3 heteroatoms selected from O, N, and S;
two R₁₅ together with the atoms to which they are attached form a (C₄-C₇)cycloalkyl or a 4- to 7-membered heterocycloalkyl ring comprising 1-2 heteroatoms selected from O, N, and S;
m and m1 are each independently 0, 1 or 2;
n1 is 0, 1, 2, or 3; and
each s and n is independently 1, 2, or 3, wherein s+n is ≤4;
or a pharmaceutically acceptable salt, hydrate, solvate, prodrug, stereoisomer, and tautomer thereof.

In some embodiments, the compound has a Formula (I),

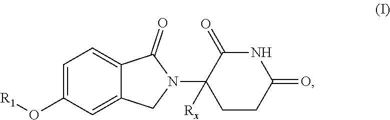

wherein:
Rₓ is H or D;
R¹ is

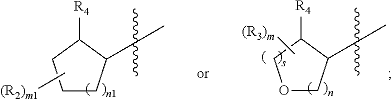

each R₂ is independently at each occurrence (C₁-C₆)alkyl, (C₁-C₆)alkoxy, (C₁-C₆)haloalkyl, (C₁-C₆)haloalkoxy, halogen, —CN, —OH, or —NH₂; or
two R₂ together with the carbon atoms to which they are attached form a (C₃-C₇)cycloalkyl or a 4- to 7-membered heterocycloalkyl ring comprising 1-3 heteroatoms selected from O, N, and S; or two R₂ together when on adjacent carbon atoms form a phenyl or a 5- or 6-membered heteroaryl ring comprising 1-3 heteroatoms selected from O, N, and S; or
R₂ and R₆ together with the carbon and nitrogen atoms to which they are attached form a 5- or 6-membered heterocycloalkyl ring optionally comprising 1-2 additional heteroatoms selected from O, N, and S, and optionally substituted with one to four substituents each independently selected from (C₁-C₆)alkyl, (C₁-C₆)haloalkyl, halogen, —OH, —CN, and —NH₂;
each R₃ is (C₁-C₆)alkyl, (C₁-C₆)alkoxy, (C₁-C₆)haloalkyl, (C₁-C₆)haloalkoxy, halogen, —OH, or —NH₂;
R₄ is —OR₅ or —NR₆R₆;
R₅ is H, (C₁-C₆)alkyl, (C₁-C₆)haloalkyl, (C₃-C₇)cycloalkyl, 5- or 6-membered heterocycloalkyl comprising 1-3 heteroatoms selected from O, N, and S, (C₆-C₁₀)aryl, or 5- or 6-membered heteroaryl comprising 1-3 heteroatoms selected from O, N, and S, wherein the alkyl is optionally substituted with one to three substituents independently selected from (C₆-C₁₀)aryl and 5- or 6-membered heteroaryl comprising 1-3 heteroatoms selected from O, N, and S;
R₆ and R₆, are each independently H, (C₁-C₆)alkyl, (C₁-C₆)haloalkyl, (C₃-C₇)cycloalkyl, 5- or 6-membered heterocycloalkyl comprising 1-3 heteroatoms selected from O, N, and S, (C₆-C₁₀)aryl, or 5- or 6-membered heteroaryl comprising 1-3 heteroatoms selected from O, N, and S, wherein the alkyl is optionally substituted with one to three R₇; or
R₆ and R₆, together with the nitrogen atom to which they are attached form a 4- to 7-membered heterocycloalkyl ring optionally comprising 1-2 additional heteroatoms selected from O, N, and S, and optionally substituted with one to four R₈; or
R₂ and R₆ together with the carbon and nitrogen atoms to which they are attached form a 5- or 6-membered heterocycloalkyl ring optionally comprising 1-2 additional heteroatoms selected from O, N, and S, and optionally substituted with one to four substituents each independently selected from (C₁-C₆)alkyl, (C₁-C₆)haloalkyl, halogen, —OH, —CN, and —NH₂;
each R₇ is (C₃-C₇)cycloalkyl, 4- to 7-membered heterocycloalkyl ring comprising 1-3 heteroatoms selected from O, N, and S, (C₆-C₁₀)aryl, or 5- or 6-membered heteroaryl comprising 1-3 heteroatoms selected from O, N, and S, wherein the cycloalkyl, heterocycloalkyl, aryl, and heteroaryl are optionally substituted with one to four R₉;
each R₈ is independently at each occurrence halogen, (C₁-C₆)alkyl, (C₁-C₆)alkoxy, (C₁-C₆)haloalkyl, (C₁-C₆)haloalkoxy, —CN, —OH, or —NH₂, wherein the alkoxy is optionally substituted with one to three substituents independently selected from (C₃-C₇)cycloalkyl, 4- to 7-membered heterocycloalkyl ring comprising 1-3 heteroatoms selected from O, N, and S, (C₆-C₁₀)aryl, and 5- or 6-membered heteroaryl comprising 1-3 heteroatoms selected from O, N, and S; or
two R₈ together with the atoms to which they are attached form a (C₅-C₇)cycloalkyl or a 4- to 7-membered heterocycloalkyl ring comprising 1-2 heteroatoms selected from O, N, and S;
each R₉ is independently at each occurrence (C₁-C₆)alkyl, (C₁-C₆)haloalkyl, halogen, —OH, —CN, or —NH₂; or
two R₉ together with the atoms to which they are attached form a (C₅-C₇)cycloalkyl or a 5- to 7-membered heterocycloalkyl ring comprising 1-2 heteroatoms selected from O, N, and S optionally substituted with one or more substituents each independently selected from (C₁-C₆)alkyl, (C₁-C₆)haloalkyl, halogen, —OH, —CN, and —NH₂;
m and m1 are each independently 0, 1, or 2;
n1 is 0, 1, 2, or 3; and
each s and n is independently 1, 2, or 3, wherein s+n is ≤4;
or a pharmaceutically acceptable salt, hydrate, solvate, prodrug, stereoisomer, and tautomer thereof.

In some embodiments, R₁ is

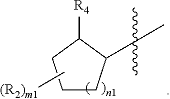

In some embodiments, n1 is 0, 1, or 2. In some embodiments, m1 is 0. In some embodiments, m1 is 2.

In some embodiments, R₁ is

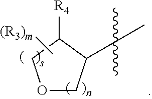

In some embodiments, n is 2 and s is 1 or 2. In some embodiments, m is 0 or 1.

In some embodiments, the compound has a Formula (Ia) or Formula (Ib):

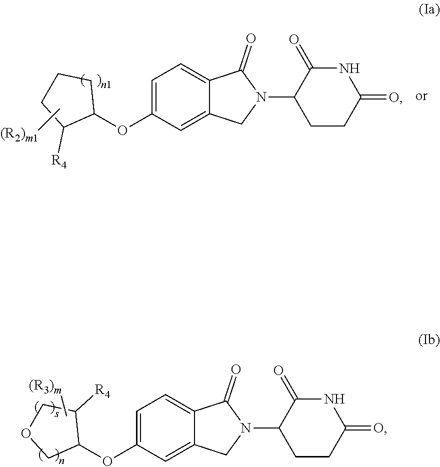

or a pharmaceutically acceptable salt, hydrate, solvate, prodrug, stereoisomer, and tautomer thereof.

In some embodiments, the compound has a Formula (Ic), Formula (Id), Formula (Ie), Formula (If), Formula (Ig), Formula (Ih), Formula (Ii), or Formula (Ij):

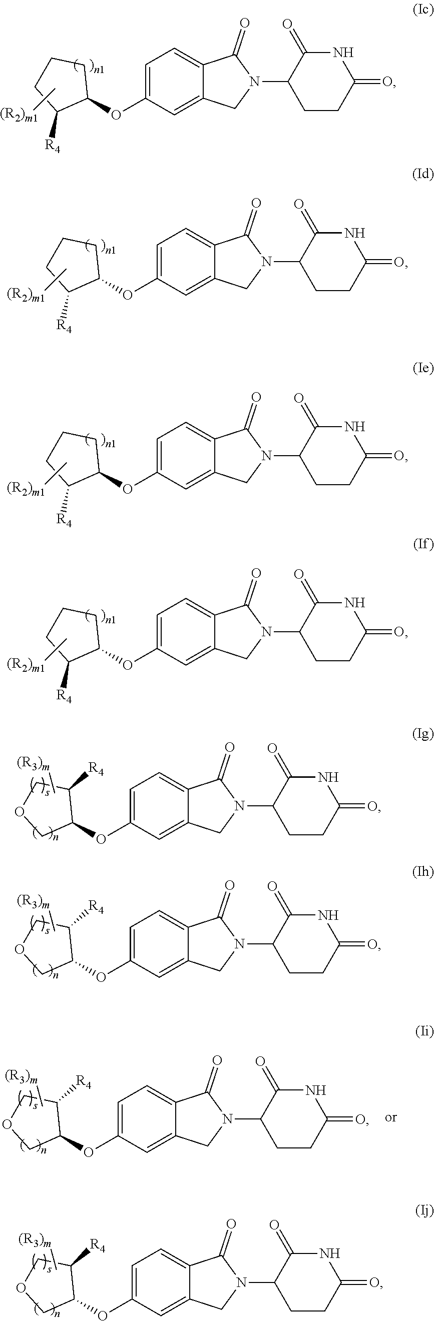

or a pharmaceutically acceptable salt, hydrate, solvate, prodrug, stereoisomer, and tautomer thereof.

In some embodiments, the compound has a Formula (Ik), Formula (Il), Formula (Im), Formula (In), Formula (Io), or Formula (Ip):

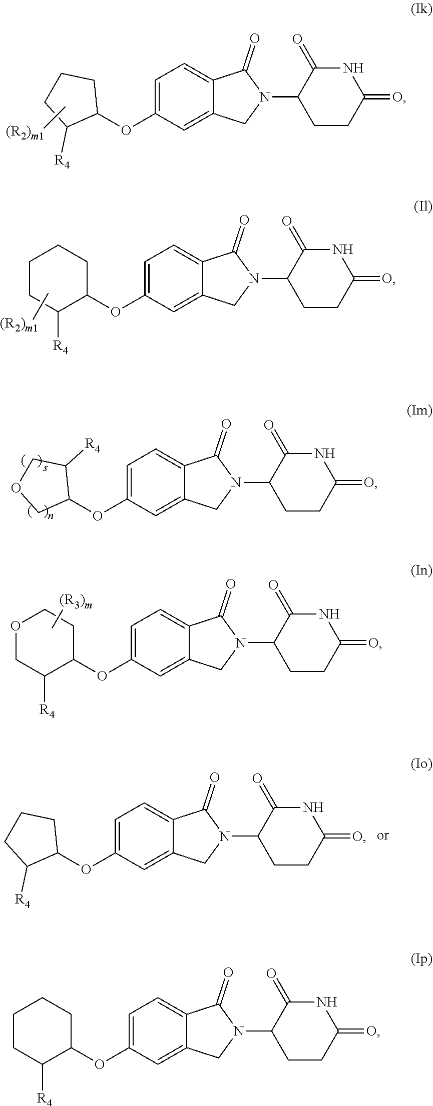

or a pharmaceutically acceptable salt, hydrate, solvate, prodrug, stereoisomer, and tautomer thereof.

In some embodiments, R₄ is —OR₅. In some embodiments, R₄ is —NR₆R₆.

In some embodiments, the compound has a Formula (Iq), Formula (Ir), Formula (Is), or Formula (It):

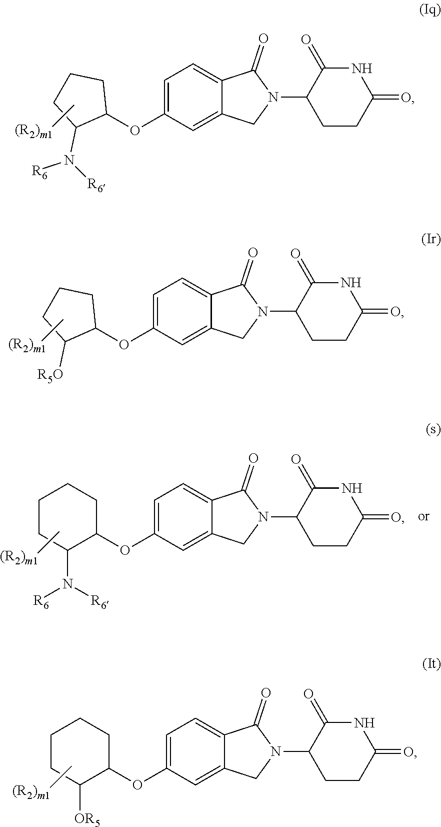

or a pharmaceutically acceptable salt, hydrate, solvate, prodrug, stereoisomer, and tautomer thereof.

In some embodiments, the compound is selected from:
3-(5-(((1S,2S)-2-((2,2-difluoroethyl)(ethyl)amino)cyclopentyl)oxy)-1-oxoisoindolin-2-yl)piperidine-2,6-dione;
3-(5-(((1S,2R)-2-((2,2-difluoroethyl)(ethyl)amino)cyclopentyl)oxy)-1-oxoisoindolin-2-yl)piperidine-2,6-dione;
3-(5-(((1R,2S)-2-((2,2-difluoroethyl)(ethyl)amino)cyclopentyl)oxy)-1-oxoisoindolin-2-yl)piperidine-2,6-dione;
3-(5-(((1R,2R)-2-((2,2-difluoroethyl)(ethyl)amino)cyclopentyl)oxy)-1-oxoisoindolin-2-yl)piperidine-2,6-dione;
3-(5-((2-((2,2-difluoroethyl)(ethyl)amino)cyclopentyl)oxy)-1-oxoisoindolin-2-yl)piperidine-2,6-dione;
3-(5-(((1S,2R)-2-(benzylamino)cyclohexyl)oxy)-1-oxoisoindolin-2-yl)piperidine-2,6-dione;
3-(5-(((1S,2S)-2-(benzylamino)cyclohexyl)oxy)-1-oxoisoindolin-2-yl)piperidine-2,6-dione;
3-(5-(((1R,2S)-2-(benzylamino)cyclohexyl)oxy)-1-oxoisoindolin-2-yl)piperidine-2,6-dione;
3-(5-(((1R,2R)-2-(benzylamino)cyclohexyl)oxy)-1-oxoisoindolin-2-yl)piperidine-2,6-dione;
3-(5-((2-(benzylamino)cyclohexyl)oxy)-1-oxoisoindolin-2-yl)piperidine-2,6-dione;
3-(5-((1-methyloctahydrocyclopenta[b]pyrrol-6-yl)oxy)-1-oxoisoindolin-2-yl)piperidine-2,6-dione;
3-(5-(((1S,2S)-2-methoxycyclohexyl)oxy)-1-oxoisoindolin-2-yl)piperidine-2,6-dione;
3-(5-(((1S,2R)-2-methoxycyclohexyl)oxy)-1-oxoisoindolin-2-yl)piperidine-2,6-dione;
3-(5-(((1R,2S)-2-methoxycyclohexyl)oxy)-1-oxoisoindolin-2-yl)piperidine-2,6-dione;
3-(5-(((1R,2R)-2-methoxycyclohexyl)oxy)-1-oxoisoindolin-2-yl)piperidine-2,6-dione;
3-(5-((2-methoxycyclohexyl)oxy)-1-oxoisoindolin-2-yl)piperidine-2,6-dione;
3-(5-(((1S,2S)-2-(3-hydroxyazetidin-1-yl)cyclopentyl)oxy)-1-oxoisoindolin-2-yl)piperidine-2,6-dione;
3-(5-(((1S,2R)-2-(3-hydroxyazetidin-1-yl)cyclopentyl)oxy)-1-oxoisoindolin-2-yl)piperidine-2,6-dione;
3-(5-(((1R,2S)-2-(3-hydroxyazetidin-1-yl)cyclopentyl)oxy)-1-oxoisoindolin-2-yl)piperidine-2,6-dione;
3-(5-(((1R,2R)-2-(3-hydroxyazetidin-1-yl)cyclopentyl)oxy)-1-oxoisoindolin-2-yl)piperidine-2,6-dione;
3-(5-((2-(3-hydroxyazetidin-1-yl)cyclopentyl)oxy)-1-oxoisoindolin-2-yl)piperidine-2,6-dione;
3-(5-(((1R,2R)-2-hydroxycyclopentyl)oxy)-1-oxoisoindolin-2-yl)piperidine-2,6-dione;
3-(5-(((1R,2S)-2-hydroxycyclopentyl)oxy)-1-oxoisoindolin-2-yl)piperidine-2,6-dione;
3-(5-(((1S,2R)-2-hydroxycyclopentyl)oxy)-1-oxoisoindolin-2-yl)piperidine-2,6-dione;
3-(5-(((1S,2S)-2-hydroxycyclopentyl)oxy)-1-oxoisoindolin-2-yl)piperidine-2,6-dione;
3-(5-((2-hydroxycyclopentyl)oxy)-1-oxoisoindolin-2-yl)piperidine-2,6-dione;
3-(5-(((1S,2S)-2-(isobutylamino)cyclohexyl)oxy)-1-oxoisoindolin-2-yl)piperidine-2,6-dione;
3-(5-(((1S,2R)-2-(isobutylamino)cyclohexyl)oxy)-1-oxoisoindolin-2-yl)piperidine-2,6-dione;
3-(5-(((1R,2S)-2-(isobutylamino)cyclohexyl)oxy)-1-oxoisoindolin-2-yl)piperidine-2,6-dione;
3-(5-(((1R,2R)-2-(isobutylamino)cyclohexyl)oxy)-1-oxoisoindolin-2-yl)piperidine-2,6-dione;
3-(5-((2-(isobutylamino)cyclohexyl)oxy)-1-oxoisoindolin-2-yl)piperidine-2,6-dione;
3-(5-(((1S,2S)-2-(4,4-difluoropiperidin-1-yl)cyclopentyl)oxy)-1-oxoisoindolin-2-yl)piperidine-2,6-dione;
3-(5-(((1S,2S)-2-(4,4-difluoropiperidin-1-yl)cyclopentyl)oxy)-1-oxoisoindolin-2-yl)piperidine-2,6-dione;
3-(5-(((1R,2R)-2-(4,4-difluoropiperidin-1-yl)cyclopentyl)oxy)-1-oxoisoindolin-2-yl)piperidine-2,6-dione;
3-(5-(((1R,2S)-2-(4,4-difluoropiperidin-1-yl)cyclopentyl)oxy)-1-oxoisoindolin-2-yl)piperidine-2,6-dione;
3-(5-((2-(4,4-difluoropiperidin-1-yl)cyclopentyl)oxy)-1-oxoisoindolin-2-yl)piperidine-2,6-dione;
3-(5-(((1S,2S)-2-(benzyloxy)cyclopentyl)oxy)-1-oxoisoindolin-2-yl)piperidine-2,6-dione;
3-(5-(((1S,2R)-2-(benzyloxy)cyclopentyl)oxy)-1-oxoisoindolin-2-yl)piperidine-2,6-dione;
3-(5-(((1R,2R)-2-(benzyloxy)cyclopentyl)oxy)-1-oxoisoindolin-2-yl)piperidine-2,6-dione;
3-(5-(((1R,2S)-2-(benzyloxy)cyclopentyl)oxy)-1-oxoisoindolin-2-yl)piperidine-2,6-dione;
3-(5-((2-(benzyloxy)cyclopentyl)oxy)-1-oxoisoindolin-2-yl)piperidine-2,6-dione;
3-(5-(((1S,2R)-2-(diethylamino)cyclopentyl)oxy)-1-oxoisoindolin-2-yl)piperidine-2,6-dione;
3-(5-(((1S,2S)-2-(diethylamino)cyclopentyl)oxy)-1-oxoisoindolin-2-yl)piperidine-2,6-dione;
3-(5-(((1R,2S)-2-(diethylamino)cyclopentyl)oxy)-1-oxoisoindolin-2-yl)piperidine-2,6-dione;
3-(5-(((1R,2R)-2-(diethylamino)cyclopentyl)oxy)-1-oxoisoindolin-2-yl)piperidine-2,6-dione;
3-(5-((2-(diethylamino)cyclopentyl)oxy)-1-oxoisoindolin-2-yl)piperidine-2,6-dione;
3-(1-oxo-5-(((1S,2S)-2-(((1-(trifluoromethyl)cyclopropyl)methyl)amino)cyclohexyl)oxy) isoindolin-2-yl)piperidine-2,6-dione;
3-(1-oxo-5-(((1R,2S)-2-(((1-(trifluoromethyl)cyclopropyl)methyl)amino)cyclohexyl)oxy) isoindolin-2-yl)piperidine-2,6-dione;
3-(1-oxo-5-(((1R,2R)-2-(((1-(trifluoromethyl)cyclopropyl)methyl)amino)cyclohexyl)oxy) isoindolin-2-yl)piperidine-2,6-dione;
3-(1-oxo-5-(((1S,2R)-2-(((1-(trifluoromethyl)cyclopropyl)methyl)amino)cyclohexyl)oxy) isoindolin-2-yl)piperidine-2,6-dione;
3-(1-oxo-5-((2-(((1-(trifluoromethyl)cyclopropyl)methyl)amino)cyclohexyl)oxy)isoindolin-2-yl)piperidine-2,6-dione;
3-(5-(((1S,2S)-2-aminocyclopentyl)oxy)-1-oxoisoindolin-2-yl)piperidine-2,6-dione;
3-(5-(((1S,2R)-2-aminocyclopentyl)oxy)-1-oxoisoindolin-2-yl)piperidine-2,6-dione;
3-(5-(((1R,2R)-2-aminocyclopentyl)oxy)-1-oxoisoindolin-2-yl)piperidine-2,6-dione;
3-(5-(((1R,2S)-2-aminocyclopentyl)oxy)-1-oxoisoindolin-2-yl)piperidine-2,6-dione;
3-(5-((2-aminocyclopentyl)oxy)-1-oxoisoindolin-2-yl)piperidine-2,6-dione;
3-(5-(((1S,2S)-2-(diethylamino)cyclohexyl)oxy)-1-oxoisoindolin-2-yl)piperidine-2,6-dione;
3-(5-(((1S,2R)-2-(diethylamino)cyclohexyl)oxy)-1-oxoisoindolin-2-yl)piperidine-2,6-dione;
3-(5-(((1R,2R)-2-(diethylamino)cyclohexyl)oxy)-1-oxoisoindolin-2-yl)piperidine-2,6-dione;
3-(5-(((1R,2S)-2-(diethylamino)cyclohexyl)oxy)-1-oxoisoindolin-2-yl)piperidine-2,6-dione;
3-(5-((2-(diethylamino)cyclohexyl)oxy)-1-oxoisoindolin-2-yl)piperidine-2,6-dione;
3-(5-(((1S,2S)-2-aminocyclohexyl)oxy)-1-oxoisoindolin-2-yl)piperidine-2,6-dione;
3-(5-(((1S,2R)-2-aminocyclohexyl)oxy)-1-oxoisoindolin-2-yl)piperidine-2,6-dione;
3-(5-(((1R,2R)-2-aminocyclohexyl)oxy)-1-oxoisoindolin-2-yl)piperidine-2,6-dione;
3-(5-(((1R,2S)-2-aminocyclohexyl)oxy)-1-oxoisoindolin-2-yl)piperidine-2,6-dione;
3-(5-((2-aminocyclohexyl)oxy)-1-oxoisoindolin-2-yl)piperidine-2,6-dione;
3-(5-(((1S,2S)-2-(3-azabicyclo[3.2.1]octan-3-yl)cyclopentyl)oxy)-1-oxoisoindolin-2-yl)piperidine-2,6-dione;
3-(5-(((1S,2R)-2-(3-azabicyclo[3.2.1]octan-3-yl)cyclopentyl)oxy)-1-oxoisoindolin-2-yl)piperidine-2,6-dione;
3-(5-(((1R,2R)-2-(3-azabicyclo[3.2.1]octan-3-yl)cyclopentyl)oxy)-1-oxoisoindolin-2-yl)piperidine-2,6-dione;
3-(5-(((1R,2S)-2-(3-azabicyclo[3.2.1]octan-3-yl)cyclopentyl)oxy)-1-oxoisoindolin-2-yl)piperidine-2,6-dione;
3-(5-((2-(3-azabicyclo[3.2.1]octan-3-yl)cyclopentyl)oxy)-1-oxoisoindolin-2-yl)piperidine-2,6-dione;
3-(5-(((1S,2S)-2-(1,4-dioxa-8-azaspiro[4.5]decan-8-yl)cyclopentyl)oxy)-1-oxoisoindolin-2-yl)piperidine-2,6-dione;
3-(5-(((1S,2R)-2-(1,4-dioxa-8-azaspiro[4.5]decan-8-yl)cyclopentyl)oxy)-1-oxoisoindolin-2-yl)piperidine-2,6-dione;
3-(5-(((1R,2R)-2-(1,4-dioxa-8-azaspiro[4.5]decan-8-yl)cyclopentyl)oxy)-1-oxoisoindolin-2-yl)piperidine-2,6-dione;
3-(5-(((1R,2S)-2-(1,4-dioxa-8-azaspiro[4.5]decan-8-yl)cyclopentyl)oxy)-1-oxoisoindolin-2-yl)piperidine-2,6-dione;
3-(5-((2-(1,4-dioxa-8-azaspiro[4.5]decan-8-yl)cyclopentyl)oxy)-1-oxoisoindolin-2-yl)piperidine-2,6-dione;
3-(5-(((1S,2S)-2-(8-oxa-3-azabicyclo[3.2.1]octan-3-yl)cyclopentyl)oxy)-1-oxoisoindolin-2-yl)piperidine-2,6-dione;
3-(5-(((1S,2R)-2-(8-oxa-3-azabicyclo[3.2.1]octan-3-yl)cyclopentyl)oxy)-1-oxoisoindolin-2-yl)piperidine-2,6-dione;
3-(5-(((1R,2R)-2-(8-oxa-3-azabicyclo[3.2.1]octan-3-yl)cyclopentyl)oxy)-1-oxoisoindolin-2-yl)piperidine-2,6-dione;
3-(5-(((1R,2S)-2-(8-oxa-3-azabicyclo[3.2.1]octan-3-yl)cyclopentyl)oxy)-1-oxoisoindolin-2-yl)piperidine-2,6-dione;
3-(5-((2-(8-oxa-3-azabicyclo[3.2.1]octan-3-yl)cyclopentyl)oxy)-1-oxoisoindolin-2-yl)piperidine-2,6-dione;
3-(1-oxo-5-(((1S,2S)-2-phenoxycyclohexyl)oxy)isoindolin-2-yl)piperidine-2,6-dione;
3-(1-oxo-5-(((1S,2R)-2-phenoxycyclohexyl)oxy)isoindolin-2-yl)piperidine-2,6-dione;
3-(1-oxo-5-(((1R,2R)-2-phenoxycyclohexyl)oxy)isoindolin-2-yl)piperidine-2,6-dione;
3-(1-oxo-5-(((1R,2S)-2-phenoxycyclohexyl)oxy)isoindolin-2-yl)piperidine-2,6-dione;
3-(1-oxo-5-((2-phenoxycyclohexyl)oxy)isoindolin-2-yl)piperidine-2,6-dione;
3-(5-(((1S,2S)-2-(benzylamino)cyclopentyl)oxy)-1-oxoisoindolin-2-yl)piperidine-2,6-dione;
3-(5-(((1S,2R)-2-(benzylamino)cyclopentyl)oxy)-1-oxoisoindolin-2-yl)piperidine-2,6-dione;
3-(5-(((1R,2R)-2-(benzylamino)cyclopentyl)oxy)-1-oxoisoindolin-2-yl)piperidine-2,6-dione;
3-(5-(((1R,2S)-2-(benzylamino)cyclopentyl)oxy)-1-oxoisoindolin-2-yl)piperidine-2,6-dione;
3-(5-((2-(benzylamino)cyclopentyl)oxy)-1-oxoisoindolin-2-yl)piperidine-2,6-dione;
3-(5-(((3S,4S)-3-(benzylamino)tetrahydro-2H-pyran-4-yl)oxy)-1-oxoisoindolin-2-yl)piperidine-2,6-dione;
3-(5-(((3R,4S)-3-(benzylamino)tetrahydro-2H-pyran-4-yl)oxy)-1-oxoisoindolin-2-yl)piperidine-2,6-dione;
3-(5-(((3R,4R)-3-(benzylamino)tetrahydro-2H-pyran-4-yl)oxy)-1-oxoisoindolin-2-yl)piperidine-2,6-dione;
3-(5-(((3S,4R)-3-(benzylamino)tetrahydro-2H-pyran-4-yl)oxy)-1-oxoisoindolin-2-yl)piperidine-2,6-dione;
3-(5-((3-(benzylamino)tetrahydro-2H-pyran-4-yl)oxy)-1-oxoisoindolin-2-yl)piperidine-2,6-dione;
3-(5-(((1S,2S)-2-(benzylamino)cyclohexyl)oxy)-1-oxoisoindolin-2-yl)piperidine-2,6-dione;
3-(5-(((1S,2R)-2-(benzylamino)cyclohexyl)oxy)-1-oxoisoindolin-2-yl)piperidine-2,6-dione;
3-(5-(((1R,2R)-2-(benzylamino)cyclohexyl)oxy)-1-oxoisoindolin-2-yl)piperidine-2,6-dione;
3-(5-(((1R,2S)-2-(benzylamino)cyclohexyl)oxy)-1-oxoisoindolin-2-yl)piperidine-2,6-dione;
3-(5-((2-(benzylamino)cyclohexyl)oxy)-1-oxoisoindolin-2-yl)piperidine-2,6-dione;
3-(1-oxo-5-(((1S,2S)-2-(((R)-1-phenylethyl)amino)cyclohexyl)oxy)isoindolin-2-yl)piperidine-2,6-dione;
3-(1-oxo-5-(((1S,2R)-2-(((R)-1-phenylethyl)amino)cyclohexyl)oxy)isoindolin-2-yl)piperidine-2,6-dione;
3-(1-oxo-5-(((1R,2R)-2-(((R)-1-phenylethyl)amino)cyclohexyl)oxy)isoindolin-2-yl)piperidine-2,6-dione;
3-(1-oxo-5-(((1R,2S)-2-(((R)-1-phenylethyl)amino)cyclohexyl)oxy)isoindolin-2-yl)piperidine-2,6-dione;
3-(1-oxo-5-((2-(((R)-1-phenylethyl)amino)cyclohexyl)oxy)isoindolin-2-yl)piperidine-2,6-dione;
3-(5-(((1S,2S)-2-(ethyl(2-fluoroethyl)amino)cyclopentyl)oxy)-1-oxoisoindolin-2-yl)piperidine-2,6-dione;
3-(5-(((1S,2R)-2-(ethyl(2-fluoroethyl)amino)cyclopentyl)oxy)-1-oxoisoindolin-2-yl)piperidine-2,6-dione;
3-(5-(((1R,2R)-2-(ethyl(2-fluoroethyl)amino)cyclopentyl)oxy)-1-oxoisoindolin-2-yl)piperidine-2,6-dione;
3-(5-(((1R,2S)-2-(ethyl(2-fluoroethyl)amino)cyclopentyl)oxy)-1-oxoisoindolin-2-yl)piperidine-2,6-dione;
3-(5-((2-(ethyl(2-fluoroethyl)amino)cyclopentyl)oxy)-1-oxoisoindolin-2-yl)piperidine-2,6-dione;
3-(5-(((1S,2S)-2-(ethyl(isopropyl)amino)cyclopentyl)oxy)-1-oxoisoindolin-2-yl)piperidine-2,6-dione;
3-(5-(((1S,2R)-2-(ethyl(isopropyl)amino)cyclopentyl)oxy)-1-oxoisoindolin-2-yl)piperidine-2,6-dione;
3-(5-(((1R,2R)-2-(ethyl(isopropyl)amino)cyclopentyl)oxy)-1-oxoisoindolin-2-yl)piperidine-2,6-dione;
3-(5-(((1R,2S)-2-(ethyl(isopropyl)amino)cyclopentyl)oxy)-1-oxoisoindolin-2-yl)piperidine-2,6-dione;
3-(5-((2-(ethyl(isopropyl)amino)cyclopentyl)oxy)-1-oxoisoindolin-2-yl)piperidine-2,6-dione;
3-(5-(((1S,2S)-2-methoxycyclopentyl)oxy)-1-oxoisoindolin-2-yl)piperidine-2,6-dione;
3-(5-(((1S,2R)-2-methoxycyclopentyl)oxy)-1-oxoisoindolin-2-yl)piperidine-2,6-dione;
3-(5-(((1R,2R)-2-methoxycyclopentyl)oxy)-1-oxoisoindolin-2-yl)piperidine-2,6-dione;
3-(5-(((1R,2S)-2-methoxycyclopentyl)oxy)-1-oxoisoindolin-2-yl)piperidine-2,6-dione;
3-(5-((2-methoxycyclopentyl)oxy)-1-oxoisoindolin-2-yl)piperidine-2,6-dione;
3-(5-(((1S,2S)-2-hydroxycyclohexyl)oxy)-1-oxoisoindolin-2-yl)piperidine-2,6-dione;
3-(5-(((1S,2R)-2-hydroxycyclohexyl)oxy)-1-oxoisoindolin-2-yl)piperidine-2,6-dione;
3-(5-(((1R,2R)-2-hydroxycyclohexyl)oxy)-1-oxoisoindolin-2-yl)piperidine-2,6-dione;
3-(5-(((1R,2S)-2-hydroxycyclohexyl)oxy)-1-oxoisoindolin-2-yl)piperidine-2,6-dione;
3-(5-((2-hydroxycyclohexyl)oxy)-1-oxoisoindolin-2-yl)piperidine-2,6-dione;
3-(5-(((1S,2S)-2-(ethylamino)cyclopentyl)oxy)-1-oxoisoindolin-2-yl)piperidine-2,6-dione;
3-(5-(((1S,2R)-2-(ethylamino)cyclopentyl)oxy)-1-oxoisoindolin-2-yl)piperidine-2,6-dione;
3-(5-(((1R,2R)-2-(ethylamino)cyclopentyl)oxy)-1-oxoisoindolin-2-yl)piperidine-2,6-dione;
3-(5-(((1R,2S)-2-(ethylamino)cyclopentyl)oxy)-1-oxoisoindolin-2-yl)piperidine-2,6-dione;
3-(5-((2-(ethylamino)cyclopentyl)oxy)-1-oxoisoindolin-2-yl)piperidine-2,6-dione;
3-(5-(((1S,2S)-2-(bis(cyclopropylmethyl)amino)cyclopentyl)oxy)-1-oxoisoindolin-2-yl)piperidine-2,6-dione;
3-(5-(((1R,2S)-2-(bis(cyclopropylmethyl)amino)cyclopentyl)oxy)-1-oxoisoindolin-2-yl)piperidine-2,6-dione;
3-(5-(((1R,2R)-2-(bis(cyclopropylmethyl)amino)cyclopentyl)oxy)-1-oxoisoindolin-2-yl)piperidine-2,6-dione;
3-(5-(((1S,2R)-2-(bis(cyclopropylmethyl)amino)cyclopentyl)oxy)-1-oxoisoindolin-2-yl)piperidine-2,6-dione;
3-(5-((2-(bis(cyclopropylmethyl)amino)cyclopentyl)oxy)-1-oxoisoindolin-2-yl)piperidine-2,6-dione;
3-(1-oxo-5-(((1S,2S)-2-(piperidin-1-yl)cyclopentyl)oxy)isoindolin-2-yl)piperidine-2,6-dione;
3-(1-oxo-5-(((1S,2R)-2-(piperidin-1-yl)cyclopentyl)oxy)isoindolin-2-yl)piperidine-2,6-dione;
3-(1-oxo-5-(((1R,2R)-2-(piperidin-1-yl)cyclopentyl)oxy)isoindolin-2-yl)piperidine-2,6-dione;
3-(1-oxo-5-(((1R,2S)-2-(piperidin-1-yl)cyclopentyl)oxy)isoindolin-2-yl)piperidine-2,6-dione;
3-(1-oxo-5-((2-(piperidin-1-yl)cyclopentyl)oxy)isoindolin-2-yl)piperidine-2,6-dione;
3-(5-(((1S,2S)-2-morpholinocyclopentyl)oxy)-1-oxoisoindolin-2-yl)piperidine-2,6-dione;
3-(5-(((1S,2R)-2-morpholinocyclopentyl)oxy)-1-oxoisoindolin-2-yl)piperidine-2,6-dione;
3-(5-(((1R,2R)-2-morpholinocyclopentyl)oxy)-1-oxoisoindolin-2-yl)piperidine-2,6-dione;
3-(5-(((1R,2S)-2-morpholinocyclopentyl)oxy)-1-oxoisoindolin-2-yl)piperidine-2,6-dione;
3-(5-((2-morpholinocyclopentyl)oxy)-1-oxoisoindolin-2-yl)piperidine-2,6-dione;
3-(5-(((1S,2S)-2-(dibenzylamino)cyclohexyl)oxy)-1-oxoisoindolin-2-yl)piperidine-2,6-dione;
3-(5-(((1S,2R)-2-(dibenzylamino)cyclohexyl)oxy)-1-oxoisoindolin-2-yl)piperidine-2,6-dione;
3-(5-(((1R,2R)-2-(dibenzylamino)cyclohexyl)oxy)-1-oxoisoindolin-2-yl)piperidine-2,6-dione;
3-(5-(((1R,2S)-2-(dibenzylamino)cyclohexyl)oxy)-1-oxoisoindolin-2-yl)piperidine-2,6-dione;
3-(5-((2-(dibenzylamino)cyclohexyl)oxy)-1-oxoisoindolin-2-yl)piperidine-2,6-dione; cis-3-(5-((2-(diethylamino)cyclohexyl)oxy)-1-oxoisoindolin-2-yl)piperidine-2,6-dione; trans-3-(5-((2-(diethylamino)cyclohexyl)oxy)-1-oxoisoindolin-2-yl)piperidine-2,6-dione;
3-(5-(((1S,2S)-2-(methylamino)cyclohexyl)oxy)-1-oxoisoindolin-2-yl)piperidine-2,6-dione;
3-(5-(((1S,2R)-2-(methylamino)cyclohexyl)oxy)-1-oxoisoindolin-2-yl)piperidine-2,6-dione;
3-(5-(((1R,2R)-2-(methylamino)cyclohexyl)oxy)-1-oxoisoindolin-2-yl)piperidine-2,6-dione;
3-(5-(((1R,2S)-2-(methylamino)cyclohexyl)oxy)-1-oxoisoindolin-2-yl)piperidine-2,6-dione;
3-(5-((2-(methylamino)cyclohexyl)oxy)-1-oxoisoindolin-2-yl)piperidine-2,6-dione;
1-((1S,2S)-2-((2-(2,6-dioxopiperidin-3-yl)-1-oxoisoindolin-5-yl)oxy)cyclopentyl)-4-methylpiperidine-4-carbonitrile;
1-((1R,2S)-2-((2-(2,6-dioxopiperidin-3-yl)-1-oxoisoindolin-5-yl)oxy)cyclopentyl)-4-methylpiperidine-4-carbonitrile;
1-((1R,2R)-2-((2-(2,6-dioxopiperidin-3-yl)-1-oxoisoindolin-5-yl)oxy)cyclopentyl)-4-methylpiperidine-4-carbonitrile;
1-((1S,2R)-2-((2-(2,6-dioxopiperidin-3-yl)-1-oxoisoindolin-5-yl)oxy)cyclopentyl)-4-methylpiperidine-4-carbonitrile;
1-(2-((2-(2,6-dioxopiperidin-3-yl)-1-oxoisoindolin-5-yl)oxy)cyclopentyl)-4-methylpiperidine-4-carbonitrile;
3-(5-(((1S,2S)-2-(benzyl(methyl)amino)cyclohexyl)oxy)-1-oxoisoindolin-2-yl)piperidine-2,6-dione;
3-(5-(((1S,2R)-2-(benzyl(methyl)amino)cyclohexyl)oxy)-1-oxoisoindolin-2-yl)piperidine-2,6-dione;
3-(5-(((1R,2R)-2-(benzyl(methyl)amino)cyclohexyl)oxy)-1-oxoisoindolin-2-yl)piperidine-2,6-dione;
3-(5-(((1R,2S)-2-(benzyl(methyl)amino)cyclohexyl)oxy)-1-oxoisoindolin-2-yl)piperidine-2,6-dione;
3-(5-((2-(benzyl(methyl)amino)cyclohexyl)oxy)-1-oxoisoindolin-2-yl)piperidine-2,6-dione;
3-(5-(((3S,4S)-3-aminotetrahydro-2H-pyran-4-yl)oxy)-1-oxoisoindolin-2-yl)piperidine-2,6-dione;
3-(5-(((3R,4S)-3-aminotetrahydro-2H-pyran-4-yl)oxy)-1-oxoisoindolin-2-yl)piperidine-2,6-dione;
3-(5-(((3R,4R)-3-aminotetrahydro-2H-pyran-4-yl)oxy)-1-oxoisoindolin-2-yl)piperidine-2,6-dione;
3-(5-(((3S,4R)-3-aminotetrahydro-2H-pyran-4-yl)oxy)-1-oxoisoindolin-2-yl)piperidine-2,6-dione;
3-(5-((3-aminotetrahydro-2H-pyran-4-yl)oxy)-1-oxoisoindolin-2-yl)piperidine-2,6-dione;
3-(5-((1S,2S)-2-(benzylamino)cyclobutoxy)-1-oxoisoindolin-2-yl)piperidine-2,6-dione;
3-(5-((1R,2R)-2-(benzylamino)cyclobutoxy)-1-oxoisoindolin-2-yl)piperidine-2,6-dione;
3-(5-((1S,2R)-2-(benzylamino)cyclobutoxy)-1-oxoisoindolin-2-yl)piperidine-2,6-dione;
3-(5-((1R,2S)-2-(benzylamino)cyclobutoxy)-1-oxoisoindolin-2-yl)piperidine-2,6-dione;
3-(5-(2-(benzylamino)cyclobutoxy)-1-oxoisoindolin-2-yl)piperidine-2,6-dione;
3-(5-(((3R,4S)-4-aminotetrahydrofuran-3-yl)oxy)-1-oxoisoindolin-2-yl)piperidine-2,6-dione;
3-(5-(((3R,4R)-4-aminotetrahydrofuran-3-yl)oxy)-1-oxoisoindolin-2-yl)piperidine-2,6-dione;
3-(5-(((3S,4R)-4-aminotetrahydrofuran-3-yl)oxy)-1-oxoisoindolin-2-yl)piperidine-2,6-dione;
3-(5-(((3S,4S)-4-aminotetrahydrofuran-3-yl)oxy)-1-oxoisoindolin-2-yl)piperidine-2,6-dione;
3-(5-((4-aminotetrahydrofuran-3-yl)oxy)-1-oxoisoindolin-2-yl)piperidine-2,6-dione;
3-(5-(((3R,4S)-4-(diethylamino)tetrahydrofuran-3-yl)oxy)-1-oxoisoindolin-2-yl)piperidine-2,6-dione;
3-(5-(((3R,4R)-4-(diethylamino)tetrahydrofuran-3-yl)oxy)-1-oxoisoindolin-2-yl)piperidine-2,6-dione;
3-(5-(((3S,4R)-4-(diethylamino)tetrahydrofuran-3-yl)oxy)-1-oxoisoindolin-2-yl)piperidine-2,6-dione;
3-(5-(((3S,4S)-4-(diethylamino)tetrahydrofuran-3-yl)oxy)-1-oxoisoindolin-2-yl)piperidine-2,6-dione;
3-(5-((4-(diethylamino)tetrahydrofuran-3-yl)oxy)-1-oxoisoindolin-2-yl)piperidine-2,6-dione;
3-(5-(((1S,2S)-2-(ethylamino)cyclohexyl)oxy)-1-oxoisoindolin-2-yl)piperidine-2,6-dione;
3-(5-(((1S,2R)-2-(ethylamino)cyclohexyl)oxy)-1-oxoisoindolin-2-yl)piperidine-2,6-dione;
3-(5-(((1R,2R)-2-(ethylamino)cyclohexyl)oxy)-1-oxoisoindolin-2-yl)piperidine-2,6-dione;
3-(5-(((1R,2S)-2-(ethylamino)cyclohexyl)oxy)-1-oxoisoindolin-2-yl)piperidine-2,6-dione;
3-(5-((2-(ethylamino)cyclohexyl)oxy)-1-oxoisoindolin-2-yl)piperidine-2,6-dione;
4-((((1S,2S)-2-((2-(2,6-dioxopiperidin-3-yl)-1-oxoisoindolin-5-yl)oxy)cyclohexyl)amino) methyl)benzonitrile;
4-((((1R,2S)-2-((2-(2,6-dioxopiperidin-3-yl)-1-oxoisoindolin-5-yl)oxy)cyclohexyl)amino)methyl)benzonitrile;
4-((((1R,2R)-2-((2-(2,6-dioxopiperidin-3-yl)-1-oxoisoindolin-5-yl)oxy)cyclohexyl)amino)methyl)benzonitrile;
4-((((1S,2R)-2-((2-(2,6-dioxopiperidin-3-yl)-1-oxoisoindolin-5-yl)oxy)cyclohexyl)amino)methyl)benzonitrile;
4-(((2-((2-(2,6-dioxopiperidin-3-yl)-1-oxoisoindolin-5-yl)oxy)cyclohexyl)amino)methyl)benzonitrile;
3-(5-((1S,2S)-2-(diethylamino)cyclobutoxy)-1-oxoisoindolin-2-yl)piperidine-2,6-dione;
3-(5-((1R,2S)-2-(diethylamino)cyclobutoxy)-1-oxoisoindolin-2-yl)piperidine-2,6-dione;
3-(5-((1R,2R)-2-(diethylamino)cyclobutoxy)-1-oxoisoindolin-2-yl)piperidine-2,6-dione;
3-(5-((1S,2R)-2-(diethylamino)cyclobutoxy)-1-oxoisoindolin-2-yl)piperidine-2,6-dione;
3-(5-(2-(diethylamino)cyclobutoxy)-1-oxoisoindolin-2-yl)piperidine-2,6-dione;
3-(5-(((1S,2S)-2-(ethyl((3-fluorobicyclo[1.1.1]pentan-1-yl)methyl)amino)cyclopentyl)oxy)-1-oxoisoindolin-2-yl)piperidine-2,6-dione;
3-(5-(((1S,2R)-2-(ethyl((3-fluorobicyclo[1.1.1]pentan-1-yl)methyl)amino)cyclopentyl)oxy)-1-oxoisoindolin-2-yl)piperidine-2,6-dione;
3-(5-(((1R,2R)-2-(ethyl((3-fluorobicyclo[1.1.1]pentan-1-yl)methyl)amino)cyclopentyl)oxy)-1-oxoisoindolin-2-yl)piperidine-2,6-dione;
3-(5-(((1R,2S)-2-(ethyl((3-fluorobicyclo[1.1.1]pentan-1-yl)methyl)amino)cyclopentyl)oxy)-1-oxoisoindolin-2-yl)piperidine-2,6-dione;
3-(5-((2-(ethyl((3-fluorobicyclo[1.1.1]pentan-1-yl)methyl)amino)cyclopentyl)oxy)-1-oxoisoindolin-2-yl)piperidine-2,6-dione;
3-((((1S,2S)-2-((2-(2,6-dioxopiperidin-3-yl)-1-oxoisoindolin-5-yl)oxy)cyclohexyl)amino)methyl)benzonitrile;
3-((((1R,2S)-2-((2-(2,6-dioxopiperidin-3-yl)-1-oxoisoindolin-5-yl)oxy)cyclohexyl)amino)methyl)benzonitrile;
3-((((1R,2R)-2-((2-(2,6-dioxopiperidin-3-yl)-1-oxoisoindolin-5-yl)oxy)cyclohexyl)amino)methyl)benzonitrile;
3-((((1S,2R)-2-((2-(2,6-dioxopiperidin-3-yl)-1-oxoisoindolin-5-yl)oxy)cyclohexyl)amino)methyl)benzonitrile;
3-(((2-((2-(2,6-dioxopiperidin-3-yl)-1-oxoisoindolin-5-yl)oxy)cyclohexyl)amino)methyl)benzonitrile;
3-(5-(((1S,2S)-2-(isopropylamino)cyclohexyl)oxy)-1-oxoisoindolin-2-yl)piperidine-2,6-dione;
3-(5-(((1S,2R)-2-(isopropylamino)cyclohexyl)oxy)-1-oxoisoindolin-2-yl)piperidine-2,6-dione;
3-(5-(((1R,2S)-2-(isopropylamino)cyclohexyl)oxy)-1-oxoisoindolin-2-yl)piperidine-2,6-dione;
3-(5-(((1R,2R)-2-(isopropylamino)cyclohexyl)oxy)-1-oxoisoindolin-2-yl)piperidine-2,6-dione;
3-(5-((2-(isopropylamino)cyclohexyl)oxy)-1-oxoisoindolin-2-yl)piperidine-2,6-dione;
2-((((1S,2S)-2-((2-(2,6-dioxopiperidin-3-yl)-1-oxoisoindolin-5-yl)oxy)cyclohexyl)amino)methyl)benzonitrile;
2-((((1R,2S)-2-((2-(2,6-dioxopiperidin-3-yl)-1-oxoisoindolin-5-yl)oxy)cyclohexyl)amino)methyl)benzonitrile;
2-((((1R,2R)-2-((2-(2,6-dioxopiperidin-3-yl)-1-oxoisoindolin-5-yl)oxy)cyclohexyl)amino)methyl)benzonitrile;
2-((((1S,2R)-2-((2-(2,6-dioxopiperidin-3-yl)-1-oxoisoindolin-5-yl)oxy)cyclohexyl)amino)methyl)benzonitrile;
2-(((2-((2-(2,6-dioxopiperidin-3-yl)-1-oxoisoindolin-5-yl)oxy)cyclohexyl)amino)methyl)benzonitrile;
3-(5-(((1S,2S)-2-(((3-fluorobicyclo[1.1.1]pentan-1-yl)methyl)amino)cyclohexyl)oxy)-1-oxoisoindolin-2-yl)piperidine-2,6-dione;
3-(5-(((1R,2S)-2-(((3-fluorobicyclo[1.1.1]pentan-1-yl)methyl)amino)cyclohexyl)oxy)-1-oxoisoindolin-2-yl)piperidine-2,6-dione;
3-(5-(((1R,2R)-2-(((3-fluorobicyclo[1.1.1]pentan-1-yl)methyl)amino)cyclohexyl)oxy)-1-oxoisoindolin-2-yl)piperidine-2,6-dione;
3-(5-(((1S,2R)-2-(((3-fluorobicyclo[1.1.1]pentan-1-yl)methyl)amino)cyclohexyl)oxy)-1-oxoisoindolin-2-yl)piperidine-2,6-dione;
3-(5-((2-(((3-fluorobicyclo[1.1.1]pentan-1-yl)methyl)amino)cyclohexyl)oxy)-1-oxoisoindolin-2-yl)piperidine-2,6-dione;
3-(5-(((1S,2S)-2-(4-hydroxy-4-methylpiperidin-1-yl)cyclopentyl)oxy)-1-oxoisoindolin-2-yl)piperidine-2,6-dione;
3-(5-(((1S,2R)-2-(4-hydroxy-4-methylpiperidin-1-yl)cyclopentyl)oxy)-1-oxoisoindolin-2-yl)piperidine-2,6-dione;
3-(5-(((1R,2R)-2-(4-hydroxy-4-methylpiperidin-1-yl)cyclopentyl)oxy)-1-oxoisoindolin-2-yl)piperidine-2,6-dione;
3-(5-(((1R,2S)-2-(4-hydroxy-4-methylpiperidin-1-yl)cyclopentyl)oxy)-1-oxoisoindolin-2-yl)piperidine-2,6-dione;
3-(5-((2-(4-hydroxy-4-methylpiperidin-1-yl)cyclopentyl)oxy)-1-oxoisoindolin-2-yl)piperidine-2,6-dione;
3-(5-(((1S,2S)-2-(4-hydroxy-4-(trifluoromethyl)piperidin-1-yl)cyclopentyl)oxy)-1-oxoisoindolin-2-yl)piperidine-2,6-dione;
3-(5-(((1S,2R)-2-(4-hydroxy-4-(trifluoromethyl)piperidin-1-yl)cyclopentyl)oxy)-1-oxoisoindolin-2-yl)piperidine-2,6-dione;
3-(5-(((1R,2R)-2-(4-hydroxy-4-(trifluoromethyl)piperidin-1-yl)cyclopentyl)oxy)-1-oxoisoindolin-2-yl)piperidine-2,6-dione;
3-(5-(((1R,2S)-2-(4-hydroxy-4-(trifluoromethyl)piperidin-1-yl)cyclopentyl)oxy)-1-oxoisoindolin-2-yl)piperidine-2,6-dione;
3-(5-((2-(4-hydroxy-4-(trifluoromethyl)piperidin-1-yl)cyclopentyl)oxy)-1-oxoisoindolin-2-yl)piperidine-2,6-dione;
1-((1S,2S)-2-((2-(2,6-dioxopiperidin-3-yl)-1-oxoisoindolin-5-yl)oxy)cyclopentyl)-4-(trifluoromethyl)piperidine-4-carbonitrile;
1-((1R,2S)-2-((2-(2,6-dioxopiperidin-3-yl)-1-oxoisoindolin-5-yl)oxy)cyclopentyl)-4-(trifluoromethyl)piperidine-4-carbonitrile;
1-((1R,2R)-2-((2-(2,6-dioxopiperidin-3-yl)-1-oxoisoindolin-5-yl)oxy)cyclopentyl)-4-(trifluoromethyl)piperidine-4-carbonitrile;
1-((1S,2R)-2-((2-(2,6-dioxopiperidin-3-yl)-1-oxoisoindolin-5-yl)oxy)cyclopentyl)-4-(trifluoromethyl)piperidine-4-carbonitrile;
1-(2-((2-(2,6-dioxopiperidin-3-yl)-1-oxoisoindolin-5-yl)oxy)cyclopentyl)-4-(trifluoromethyl)piperidine-4-carbonitrile;
3-(5-(((1S,2S)-2-(2-oxa-7-azaspiro[3.5]nonan-7-yl)cyclopentyl)oxy)-1-oxoisoindolin-2-yl)piperidine-2,6-dione;
3-(5-(((1S,2R)-2-(2-oxa-7-azaspiro[3.5]nonan-7-yl)cyclopentyl)oxy)-1-oxoisoindolin-2-yl)piperidine-2,6-dione;
3-(5-(((1R,2R)-2-(2-oxa-7-azaspiro[3.5]nonan-7-yl)cyclopentyl)oxy)-1-oxoisoindolin-2-yl)piperidine-2,6-dione;
3-(5-(((1R,2S)-2-(2-oxa-7-azaspiro[3.5]nonan-7-yl)cyclopentyl)oxy)-1-oxoisoindolin-2-yl)piperidine-2,6-dione;
3-(5-((2-(2-oxa-7-azaspiro[3.5]nonan-7-yl)cyclopentyl)oxy)-1-oxoisoindolin-2-yl)piperidine-2,6-dione;
3-(1-oxo-5-(((1S,2S)-2-(3-(2,2,2-trifluoroethoxy)azetidin-1-yl)cyclohexyl)oxy)isoindolin-2-yl)piperidine-2,6-dione;
3-(1-oxo-5-(((1R,2S)-2-(3-(2,2,2-trifluoroethoxy)azetidin-1-yl)cyclohexyl)oxy)isoindolin-2-yl)piperidine-2,6-dione;
3-(1-oxo-5-(((1S,2R)-2-(3-(2,2,2-trifluoroethoxy)azetidin-1-yl)cyclohexyl)oxy)isoindolin-2-yl)piperidine-2,6-dione;
3-(1-oxo-5-(((1R,2R)-2-(3-(2,2,2-trifluoroethoxy)azetidin-1-yl)cyclohexyl)oxy)isoindolin-2--yl)piperidine-2,6-dione; 3-(1-oxo-5-((2-(3-(2,2,2-trifluoroethoxy)azetidin-1-yl)cyclohexyl)oxy)isoindolin-2-yl)piperidine-2,6-dione;
3-(5-(((1S,2S)-2-(3-(2,2-difluoroethoxy)azetidin-1-yl)cyclohexyl)oxy)-1-oxoisoindolin-2-yl)piperidine-2,6-dione;
3-(5-(((1R,2S)-2-(3-(2,2-difluoroethoxy)azetidin-1-yl)cyclohexyl)oxy)-1-oxoisoindolin-2-yl)piperidine-2,6-dione;
3-(5-(((1S,2R)-2-(3-(2,2-difluoroethoxy)azetidin-1-yl)cyclohexyl)oxy)-1-oxoisoindolin-2-yl)piperidine-2,6-dione;
3-(5-(((1R,2R)-2-(3-(2,2-difluoroethoxy)azetidin-1-yl)cyclohexyl)oxy)-1-oxoisoindolin-2-yl)piperidine-2,6-dione;
3-(5-((2-(3-(2,2-difluoroethoxy)azetidin-1-yl)cyclohexyl)oxy)-1-oxoisoindolin-2-yl)piperidine-2,6-dione;
3-(5-(((1S,2S)-2-(3-(cyclopropylmethoxy)azetidin-1-yl)cyclohexyl)oxy)-1-oxoisoindolin-2-yl)piperidine-2,6-dione;
3-(5-(((1R,2S)-2-(3-(cyclopropylmethoxy)azetidin-1-yl)cyclohexyl)oxy)-1-oxoisoindolin-2-yl)piperidine-2,6-dione;
3-(5-(((1S,2R)-2-(3-(cyclopropylmethoxy)azetidin-1-yl)cyclohexyl)oxy)-1-oxoisoindolin-2-yl)piperidine-2,6-dione;
3-(5-(((1R,2R)-2-(3-(cyclopropylmethoxy)azetidin-1-yl)cyclohexyl)oxy)-1-oxoisoindolin-2-yl)piperidine-2,6-dione;
3-(5-((2-(3-(cyclopropylmethoxy)azetidin-1-yl)cyclohexyl)oxy)-1-oxoisoindolin-2-yl)piperidine-2,6-dione;
3-(5-(((1S,2S)-2-(3-(benzyloxy)azetidin-1-yl)cyclopentyl)oxy)-1-oxoisoindolin-2-yl)piperidine-2,6-dione;
3-(5-(((1R,2S)-2-(3-(benzyloxy)azetidin-1-yl)cyclopentyl)oxy)-1-oxoisoindolin-2-yl)piperidine-2,6-dione;
3-(5-(((1S,2R)-2-(3-(benzyloxy)azetidin-1-yl)cyclopentyl)oxy)-1-oxoisoindolin-2-yl)piperidine-2,6-dione;
3-(5-(((1R,2R)-2-(3-(benzyloxy)azetidin-1-yl)cyclopentyl)oxy)-1-oxoisoindolin-2-yl)piperidine-2,6-dione;
3-(5-((2-(3-(benzyloxy)azetidin-1-yl)cyclopentyl)oxy)-1-oxoisoindolin-2-yl)piperidine-2,6-dione;
3-(5-(((1S,2S)-2-(3-isopropoxyazetidin-1-yl)cyclohexyl)oxy)-1-oxoisoindolin-2-yl)piperidine-2,6-dione;
3-(5-(((1R,2S)-2-(3-isopropoxyazetidin-1-yl)cyclohexyl)oxy)-1-oxoisoindolin-2--yl)piperidine-2,6-dione;
3-(5-(((1S,2R)-2-(3-isopropoxyazetidin-1-yl)cyclohexyl)oxy)-1-oxoisoindolin-2-yl)piperidine-2,6-dione;
3-(5-(((1R,2R)-2-(3-isopropoxyazetidin-1-yl)cyclohexyl)oxy)-1-oxoisoindolin-2-yl)piperidine-2,6-dione;
3-(5-((2-(3-isopropoxyazetidin-1-yl)cyclohexyl)oxy)-1-oxoisoindolin-2-yl)piperidine-2,6-dione;
3-(5-(((1R,2R)-2-(3-ethoxyazetidin-1-yl)cyclohexyl)oxy)-1-oxoisoindolin-2-yl)piperidine-2,6-dione;
3-(5-(((1R,2S)-2-(3-ethoxyazetidin-1-yl)cyclohexyl)oxy)-1-oxoisoindolin-2-yl)piperidine-2,6-dione;
3-(5-(((1S,2R)-2-(3-ethoxyazetidin-1-yl)cyclohexyl)oxy)-1-oxoisoindolin-2-yl)piperidine-2,6-dione;
3-(5-(((1S,2S)-2-(3-ethoxyazetidin-1-yl)cyclohexyl)oxy)-1-oxoisoindolin-2-yl)piperidine-2,6-dione;
3-(5-((2-(3-ethoxyazetidin-1-yl)cyclohexyl)oxy)-1-oxoisoindolin-2-yl)piperidine-2,6-dione;
3-(5-(((1S,2S)-2-(3-(benzyloxy)azetidin-1-yl)cyclohexyl)oxy)-1-oxoisoindolin-2-yl)piperidine-2,6-dione;
3-(5-(((1R,2S)-2-(3-(benzyloxy)azetidin-1-yl)cyclohexyl)oxy)-1-oxoisoindolin-2-yl)piperidine-2,6-dione;
3-(5-(((1S,2R)-2-(3-(benzyloxy)azetidin-1-yl)cyclohexyl)oxy)-1-oxoisoindolin-2-yl)piperidine-2,6-dione;
3-(5-(((1R,2R)-2-(3-(benzyloxy)azetidin-1-yl)cyclohexyl)oxy)-1-oxoisoindolin-2-yl)piperidine-2,6-dione;
3-(5-((2-(3-(benzyloxy)azetidin-1-yl)cyclohexyl)oxy)-1-oxoisoindolin-2-yl)piperidine-2,6-dione;
3-(5-(((1S,2S)-2-(3-ethoxyazetidin-1-yl)cycloheptyl)oxy)-1-oxoisoindolin-2-yl)piperidine-2,6-dione;
3-(5-(((1R,2S)-2-(3-ethoxyazetidin-1-yl)cycloheptyl)oxy)-1-oxoisoindolin-2-yl)piperidine-2,6-dione;
3-(5-(((1S,2R)-2-(3-ethoxyazetidin-1-yl)cycloheptyl)oxy)-1-oxoisoindolin-2-yl)piperidine-2,6-dione;
3-(5-(((1R,2R)-2-(3-ethoxyazetidin-1-yl)cycloheptyl)oxy)-1-oxoisoindolin-2-yl)piperidine-2,6-dione;
3-(5-((2-(3-ethoxyazetidin-1-yl)cycloheptyl)oxy)-1-oxoisoindolin-2-yl)piperidine-2,6-dione;
3-(5-(((1S,2S)-2-(3-(3,3-difluorocyclobutoxy)azetidin-1-yl)cyclohexyl)oxy)-1-oxoisoindolin-2-yl)piperidine-2,6-dione;
3-(5-(((1R,2S)-2-(3-(3,3-difluorocyclobutoxy)azetidin-1-yl)cyclohexyl)oxy)-1-oxoisoindolin-2-yl)piperidine-2,6-dione;
3-(5-(((1S,2R)-2-(3-(3,3-difluorocyclobutoxy)azetidin-1-yl)cyclohexyl)oxy)-1-oxoisoindolin-2-yl)piperidine-2,6-dione;
3-(5-(((1R,2R)-2-(3-(3,3-difluorocyclobutoxy)azetidin-1-yl)cyclohexyl)oxy)-1-oxoisoindolin-2-yl)piperidine-2,6-dione;
3-(5-((2-(3-(3,3-difluorocyclobutoxy)azetidin-1-yl)cyclohexyl)oxy)-1-oxoisoindolin-2-yl)piperidine-2,6-dione;
3-(5-(((1S,2S)-2-(4-hydroxypiperidin-1-yl)cyclopentyl)oxy)-1-oxoisoindolin-2-yl)piperidine-2,6-dione;
3-(5-(((1S,2R)-2-(4-hydroxypiperidin-1-yl)cyclopentyl)oxy)-1-oxoisoindolin-2-yl)piperidine-2,6-dione;
3-(5-(((1R,2R)-2-(4-hydroxypiperidin-1-yl)cyclopentyl)oxy)-1-oxoisoindolin-2-yl)piperidine-2,6-dione;
3-(5-(((1R,2S)-2-(4-hydroxypiperidin-1-yl)cyclopentyl)oxy)-1-oxoisoindolin-2-yl)piperidine-2,6-dione;
3-(5-((2-(4-hydroxypiperidin-1-yl)cyclopentyl)oxy)-1-oxoisoindolin-2-yl)piperidine-2,6-dione;
3-(1-oxo-5-(((1S,2S)-2-(4-oxopiperidin-1-yl)cyclopentyl)oxy)isoindolin-2-yl)piperidine-2,6-dione;
3-(5-(((1S,2S)-2-(3-hydroxypyrrolidin-1-yl)cyclopentyl)oxy)-1-oxoisoindolin-2-yl)piperidine-2,6-dione;
3-(5-(((1S,2R)-2-(3-hydroxypyrrolidin-1-yl)cyclopentyl)oxy)-1-oxoisoindolin-2-yl)piperidine-2,6-dione;
3-(5-(((1R,2R)-2-(3-hydroxypyrrolidin-1-yl)cyclopentyl)oxy)-1-oxoisoindolin-2-yl)piperidine-2,6-dione;
3-(5-(((1R,2S)-2-(3-hydroxypyrrolidin-1-yl)cyclopentyl)oxy)-1-oxoisoindolin-2-yl)piperidine-2,6-dione;
3-(5-((2-(3-hydroxypyrrolidin-1-yl)cyclopentyl)oxy)-1-oxoisoindolin-2-yl)piperidine-2,6-dione;
3-(5-(((1S,2S)-2-(3-hydroxy-3-methylazetidin-1-yl)cyclopentyl)oxy)-1-oxoisoindolin-2-yl)piperidine-2,6-dione;
3-(5-(((1S,2R)-2-(3-hydroxy-3-methylazetidin-1-yl)cyclopentyl)oxy)-1-oxoisoindolin-2-yl)piperidine-2,6-dione;
3-(5-(((1R,2R)-2-(3-hydroxy-3-methylazetidin-1-yl)cyclopentyl)oxy)-1-oxoisoindolin-2--yl)piperidine-2,6-dione;
3-(5-(((1R,2S)-2-(3-hydroxy-3-methylazetidin-1-yl)cyclopentyl)oxy)-1-oxoisoindolin-2-yl)piperidine-2,6-dione;
3-(5-((2-(3-hydroxy-3-methylazetidin-1-yl)cyclopentyl)oxy)-1-oxoisoindolin-2-yl)piperidine-2,6-dione;
3-(5-(((1S,2S)-2-(isobutylamino)cyclopentyl)oxy)-1-oxoisoindolin-2-yl)piperidine-2,6-dione;
3-(5-(((1S,2R)-2-(isobutylamino)cyclopentyl)oxy)-1-oxoisoindolin-2-yl)piperidine-2,6-dione;
3-(5-(((1R,2R)-2-(isobutylamino)cyclopentyl)oxy)-1-oxoisoindolin-2-yl)piperidine-2,6-dione;
3-(5-(((1R,2S)-2-(isobutylamino)cyclopentyl)oxy)-1-oxoisoindolin-2-yl)piperidine-2,6-dione;
3-(5-((2-(isobutylamino)cyclopentyl)oxy)-1-oxoisoindolin-2-yl)piperidine-2,6-dione;
3-(5-(((1S,2S)-2-(ethyl(methyl)amino)cyclopentyl)oxy)-1-oxoisoindolin-2-yl)piperidine-2,6-dione;
3-(5-(((1S,2R)-2-(ethyl(methyl)amino)cyclopentyl)oxy)-1-oxoisoindolin-2-yl)piperidine-2,6-dione;
3-(5-(((1R,2R)-2-(ethyl(methyl)amino)cyclopentyl)oxy)-1-oxoisoindolin-2-yl)piperidine-2,6-dione;
3-(5-(((1R,2S)-2-(ethyl(methyl)amino)cyclopentyl)oxy)-1-oxoisoindolin-2-yl)piperidine-2,6-dione;
3-(5-((2-(ethyl(methyl)amino)cyclopentyl)oxy)-1-oxoisoindolin-2-yl)piperidine-2,6-dione
3-(1-oxo-5-(((1S,2S)-2-((3aR,6aS)-tetrahydro-1H-furo[3,4-c]pyrrol-5(3H)-yl)cyclopentyl)oxy)isoindolin-2-yl)piperidine-2,6-dione;
3-(1-oxo-5-(((1S,2R)-2-((3aR,6aS)-tetrahydro-1H-furo[3,4-c]pyrrol-5(3H)-yl)cyclopentyl)oxy)isoindolin-2-yl)piperidine-2,6-dione;
3-(1-oxo-5-(((1R,2R)-2-((3aR,6aS)-tetrahydro-1H-furo[3,4-c]pyrrol-5(3H)-yl)cyclopentyl)oxy)isoindolin-2-yl)piperidine-2,6-dione;
3-(1-oxo-5-(((1R,2S)-2-((3aR,6aS)-tetrahydro-1H-furo[3,4-c]pyrrol-5(3H)-yl)cyclopentyl)oxy)isoindolin-2-yl)piperidine-2,6-dione;
3-(1-oxo-5-((2-((3aR,6aS)-tetrahydro-1H-furo[3,4-c]pyrrol-5(3H)-yl)cyclopentyl)oxy)isoindolin-2-yl)piperidine-2,6-dione;
3-(1-oxo-5-((2-(tetrahydro-1H-furo[3,4-c]pyrrol-5(3H)-yl)cyclopentyl)oxy)isoindolin-2-yl)piperidine-2,6-dione;
3-(1-oxo-5-(((1S,2S)-2-((pyridin-2-ylmethyl)amino)cyclopentyl)oxy)isoindolin-2-yl)piperidine-2,6-dione;
3-(1-oxo-5-(((1S,2R)-2-((pyridin-2-ylmethyl)amino)cyclopentyl)oxy)isoindolin-2-yl)piperidine-2,6-dione;
3-(1-oxo-5-(((1R,2R)-2-((pyridin-2-ylmethyl)amino)cyclopentyl)oxy)isoindolin-2-yl)piperidine-2,6-dione
3-(1-oxo-5-(((1R,2S)-2-((pyridin-2-ylmethyl)amino)cyclopentyl)oxy)isoindolin-2-yl)piperidine-2,6-dione;
3-(1-oxo-5-((2-((pyridin-2-ylmethyl)amino)cyclopentyl)oxy)isoindolin-2-yl)piperidine-2,6-dione;
3-(1-oxo-5-(((1S,2S)-2-(pyrrolidin-1-yl)cyclopentyl)oxy)isoindolin-2-yl)piperidine-2,6-dione;
3-(1-oxo-5-(((1S,2R)-2-(pyrrolidin-1-yl)cyclopentyl)oxy)isoindolin-2-yl)piperidine-2,6-dione;
3-(1-oxo-5-(((1R,2R)-2-(pyrrolidin-1-yl)cyclopentyl)oxy)isoindolin-2-yl)piperidine-2,6-dione;
3-(1-oxo-5-(((1R,2S)-2-(pyrrolidin-1-yl)cyclopentyl)oxy)isoindolin-2-yl)piperidine-2,6-dione;
3-(1-oxo-5-((2-(pyrrolidin-1-yl)cyclopentyl)oxy)isoindolin-2-yl)piperidine-2,6-dione;
3-(5-(((1S,2S)-2-(2-oxa-6-azaspiro[3.3]heptan-6-yl)cyclopentyl)oxy)-1-oxoisoindolin-2-yl)piperidine-2,6-dione;
3-(5-(((1S,2R)-2-(2-oxa-6-azaspiro[3.3]heptan-6-yl)cyclopentyl)oxy)-1-oxoisoindolin-2-yl)piperidine-2,6-dione;
3-(5-(((1R,2R)-2-(2-oxa-6-azaspiro[3.3]heptan-6-yl)cyclopentyl)oxy)-1-oxoisoindolin-2-yl)piperidine-2,6-dione;
3-(5-(((1R,2S)-2-(2-oxa-6-azaspiro[3.3]heptan-6-yl)cyclopentyl)oxy)-1-oxoisoindolin-2-yl)piperidine-2,6-dione;
3-(5-((2-(2-oxa-6-azaspiro[3.3]heptan-6-yl)cyclopentyl)oxy)-1-oxoisoindolin-2-yl)piperidine-2,6-dione;
3-(5-(((1S,2S)-2-(bis((3-methyloxetan-3-yl)methyl)amino)cyclopentyl)oxy)-1-oxoisoindolin-2-yl)piperidine-2,6-dione;
3-(5-(((1S,2R)-2-(bis((3-methyloxetan-3-yl)methyl)amino)cyclopentyl)oxy)-1-oxoisoindolin-2-yl)piperidine-2,6-dione;
3-(5-(((1R,2R)-2-(bis((3-methyloxetan-3-yl)methyl)amino)cyclopentyl)oxy)-1-oxoisoindolin-2-yl)piperidine-2,6-dione;
3-(5-(((1R,2S)-2-(bis((3-methyloxetan-3-yl)methyl)amino)cyclopentyl)oxy)-1-oxoisoindolin-2-yl)piperidine-2,6-dione;
3-(5-((2-(bis((3-methyloxetan-3-yl)methyl)amino)cyclopentyl)oxy)-1-oxoisoindolin-2-yl)piperidine-2,6-dione;
3-(5-(((1S,2S)-2-(4-methylpiperidin-1-yl)cyclopentyl)oxy)-1-oxoisoindolin-2-yl)piperidine-2,6-dione;
3-(5-(((1S,2R)-2-(4-methylpiperidin-1-yl)cyclopentyl)oxy)-1-oxoisoindolin-2-yl)piperidine-2,6-dione;
3-(5-(((1R,2R)-2-(4-methylpiperidin-1-yl)cyclopentyl)oxy)-1-oxoisoindolin-2-yl)piperidine-2,6-dione;
3-(5-(((1R,2S)-2-(4-methylpiperidin-1-yl)cyclopentyl)oxy)-1-oxoisoindolin-2-yl)piperidine-2,6-dione;
3-(5-((2-(4-methylpiperidin-1-yl)cyclopentyl)oxy)-1-oxoisoindolin-2-yl)piperidine-2,6-dione;
3-(1-oxo-5-(((1S,2S)-2-((pyridin-3-ylmethyl)amino)cyclopentyl)oxy)isoindolin-2-yl)piperidine-2,6-dione;
3-(1-oxo-5-(((1S,2R)-2-((pyridin-3-ylmethyl)amino)cyclopentyl)oxy)isoindolin-2-yl)piperidine-2,6-dione;
3-(1-oxo-5-(((1R,2R)-2-((pyridin-3-ylmethyl)amino)cyclopentyl)oxy)isoindolin-2-yl)piperidine-2,6-dione;
3-(1-oxo-5-(((1R,2S)-2-((pyridin-3-ylmethyl)amino)cyclopentyl)oxy)isoindolin-2-yl)piperidine-2,6-dione;
3-(1-oxo-5-((2-((pyridin-3-ylmethyl)amino)cyclopentyl)oxy)isoindolin-2-yl)piperidine-2,6-dione;
3-(1-oxo-5-(((1S,2S)-2-((pyridin-4-ylmethyl)amino)cyclopentyl)oxy)isoindolin-2-yl)piperidine-2,6-dione;
3-(1-oxo-5-(((1S,2R)-2-((pyridin-4-ylmethyl)amino)cyclopentyl)oxy)isoindolin-2-yl)piperidine-2,6-dione;
3-(1-oxo-5-(((1R,2R)-2-((pyridin-4-ylmethyl)amino)cyclopentyl)oxy)isoindolin-2-yl)piperidine-2,6-dione;
3-(1-oxo-5-(((1R,2S)-2-((pyridin-4-ylmethyl)amino)cyclopentyl)oxy)isoindolin-2-yl)piperidine-2,6-dione;
3-(1-oxo-5-((2-((pyridin-4-ylmethyl)amino)cyclopentyl)oxy)isoindolin-2-yl)piperidine-2,6-dione;
3-(5-(((1S,2S)-2-(4-methoxy-4-methylpiperidin-1-yl)cyclopentyl)oxy)-1-oxoisoindolin-2-yl)piperidine-2,6-dione;
3-(5-(((1S,2R)-2-(4-methoxy-4-methylpiperidin-1-yl)cyclopentyl)oxy)-1-oxoisoindolin-2-yl)piperidine-2,6-dione;
3-(5-(((1R,2R)-2-(4-methoxy-4-methylpiperidin-1-yl)cyclopentyl)oxy)-1-oxoisoindolin-2-yl)piperidine-2,6-dione;
3-(5-(((1R,2S)-2-(4-methoxy-4-methylpiperidin-1-yl)cyclopentyl)oxy)-1-oxoisoindolin-2-yl)piperidine-2,6-dione;
3-(5-((2-(4-methoxy-4-methylpiperidin-1-yl)cyclopentyl)oxy)-1-oxoisoindolin-2-yl)piperidine-2,6-dione;
3-(5-(((1S,2S)-2-(4,4-dimethylpiperidin-1-yl)cyclopentyl)oxy)-1-oxoisoindolin-2--yl)piperidine-2,6-dione;
3-(5-(((1S,2R)-2-(4,4-dimethylpiperidin-1-yl)cyclopentyl)oxy)-1-oxoisoindolin-2-yl)piperidine-2,6-dione;
3-(5-(((1R,2R)-2-(4,4-dimethylpiperidin-1-yl)cyclopentyl)oxy)-1-oxoisoindolin-2-yl)piperidine-2,6-dione;
3-(5-(((1R,2S)-2-(4,4-dimethylpiperidin-1-yl)cyclopentyl)oxy)-1-oxoisoindolin-2-yl)piperidine-2,6-dione;
3-(5-((2-(4,4-dimethylpiperidin-1-yl)cyclopentyl)oxy)-1-oxoisoindolin-2-yl)piperidine-2,6-dione;
3-(5-(((1S,2S)-2-(4-methoxypiperidin-1-yl)cyclopentyl)oxy)-1-oxoisoindolin-2-yl)piperidine-2,6-dione;
3-(5-(((1S,2R)-2-(4-methoxypiperidin-1-yl)cyclopentyl)oxy)-1-oxoisoindolin-2-yl)piperidine-2,6-dione;
3-(5-(((1R,2R)-2-(4-methoxypiperidin-1-yl)cyclopentyl)oxy)-1-oxoisoindolin-2-yl)piperidine-2,6-dione;
3-(5-(((1R,2S)-2-(4-methoxypiperidin-1-yl)cyclopentyl)oxy)-1-oxoisoindolin-2-yl)piperidine-2,6-dione;
3-(5-((2-(4-methoxypiperidin-1-yl)cyclopentyl)oxy)-1-oxoisoindolin-2-yl)piperidine-2,6-dione;
3-(5-(((1S,2S)-2-(ethyl(oxetan-3-ylmethyl)amino)cyclopentyl)oxy)-1-oxoisoindolin-2-yl)piperidine-2,6-dione;
3-(5-(((1S,2R)-2-(ethyl(oxetan-3-ylmethyl)amino)cyclopentyl)oxy)-1-oxoisoindolin-2-yl)piperidine-2,6-dione;
3-(5-(((1R,2R)-2-(ethyl(oxetan-3-ylmethyl)amino)cyclopentyl)oxy)-1-oxoisoindolin-2-yl)piperidine-2,6-dione;
3-(5-(((1R,2S)-2-(ethyl(oxetan-3-ylmethyl)amino)cyclopentyl)oxy)-1-oxoisoindolin-2-yl)piperidine-2,6-dione;
3-(5-((2-(ethyl(oxetan-3-ylmethyl)amino)cyclopentyl)oxy)-1-oxoisoindolin-2-yl)piperidine-2,6-dione;
3-(5-(((1S,2S)-2-(isoindolin-2-yl)cyclopentyl)oxy)-1-oxoisoindolin-2-yl)piperidine-2,6-dione;
3-(5-(((1S,2R)-2-(isoindolin-2-yl)cyclopentyl)oxy)-1-oxoisoindolin-2-yl)piperidine-2,6-dione;
3-(5-(((1R,2R)-2-(isoindolin-2-yl)cyclopentyl)oxy)-1-oxoisoindolin-2-yl)piperidine-2,6-dione;
3-(5-(((1R,2S)-2-(isoindolin-2-yl)cyclopentyl)oxy)-1-oxoisoindolin-2-yl)piperidine-2,6-dione;
3-(5-((2-(isoindolin-2-yl)cyclopentyl)oxy)-1-oxoisoindolin-2-yl)piperidine-2,6-dione;
3-(5-(((1S,2S)-2-(3-methoxyazetidin-1-yl)cyclopentyl)oxy)-1-oxoisoindolin-2-yl)piperidine-2,6-dione;
3-(5-(((1S,2R)-2-(3-methoxyazetidin-1-yl)cyclopentyl)oxy)-1-oxoisoindolin-2-yl)piperidine-2,6-dione;
3-(5-(((1R,2R)-2-(3-methoxyazetidin-1-yl)cyclopentyl)oxy)-1-oxoisoindolin-2-yl)piperidine-2,6-dione;
3-(5-(((1R,2S)-2-(3-methoxyazetidin-1-yl)cyclopentyl)oxy)-1-oxoisoindolin-2-yl)piperidine-2,6-dione;
3-(5-((2-(3-methoxyazetidin-1-yl)cyclopentyl)oxy)-1-oxoisoindolin-2-yl)piperidine-2,6-dione;
3-(5-(((1S,2S)-2-(4-ethoxy-4-methylpiperidin-1-yl)cyclopentyl)oxy)-1-oxoisoindolin-2-yl)piperidine-2,6-dione;
3-(5-(((1S,2R)-2-(4-ethoxy-4-methylpiperidin-1-yl)cyclopentyl)oxy)-1-oxoisoindolin-2-yl)piperidine-2,6-dione;
3-(5-(((1R,2R)-2-(4-ethoxy-4-methylpiperidin-1-yl)cyclopentyl)oxy)-1-oxoisoindolin-2-yl)piperidine-2,6-dione;
3-(5-(((1R,2S)-2-(4-ethoxy-4-methylpiperidin-1-yl)cyclopentyl)oxy)-1-oxoisoindolin-2-yl)piperidine-2,6-dione;
3-(5-((2-(4-ethoxy-4-methylpiperidin-1-yl)cyclopentyl)oxy)-1-oxoisoindolin-2-yl)piperidine-2,6-dione;
3-(5-(((1S,2S)-2-((((1R,4S)-4-methoxycyclohexyl)methyl)amino)cyclopentyl)oxy)-1-oxoisoindolin-2-yl)piperidine-2,6-dione;
3-(5-(((1S,2R)-2-((((1R,4R)-4-methoxycyclohexyl)methyl)amino)cyclopentyl)oxy)-1-oxoisoindolin-2-yl)piperidine-2,6-dione;
3-(5-(((1R,2R)-2-((((1R,4R)-4-methoxycyclohexyl)methyl)amino)cyclopentyl)oxy)-1-oxoisoindolin-2-yl)piperidine-2,6-dione;
3-(5-(((1R,2S)-2-((((1R,4S)-4-methoxycyclohexyl)methyl)amino)cyclopentyl)oxy)-1-oxoisoindolin-2-yl)piperidine-2,6-dione;
3-(5-((2-((((1R,4R)-4-methoxycyclohexyl)methyl)amino)cyclopentyl)oxy)-1-oxoisoindolin-2-yl)piperidine-2,6-dione;
3-(5-((2-(((4-methoxycyclohexyl)methyl)amino)cyclopentyl)oxy)-1-oxoisoindolin-2-yl)piperidine-2,6-dione;
(1S,4R)-4-((((1S,2S)-2-((2-(2,6-dioxopiperidin-3-yl)-1-oxoisoindolin-5-yl)oxy)cyclopentyl)amino)methyl)cyclohexane-1-carbonitrile;
(1R,4r)-4-((((1R,2S)-2-((2-(2,6-dioxopiperidin-3-yl)-1-oxoisoindolin-5-yl)oxy)cyclopentyl)amino)methyl)cyclohexane-1-carbonitrile;
(1R,4r)-4-((((1R,2R)-2-((2-(2,6-dioxopiperidin-3-yl)-1-oxoisoindolin-5-yl)oxy)cyclopentyl)amino)methyl)cyclohexane-1-carbonitrile;
(1S,4r)-4-((((1S,2R)-2-((2-(2,6-dioxopiperidin-3-yl)-1-oxoisoindolin-5-yl)oxy)cyclopentyl)amino)methyl)cyclohexane-1-carbonitrile;
(1r,4r)-4-(((2-((2-(2,6-dioxopiperidin-3-yl)-1-oxoisoindolin-5-yl)oxy)cyclopentyl)amino)methyl)cyclohexane-1-carbonitrile;
4-(((2-((2-(2,6-dioxopiperidin-3-yl)-1-oxoisoindolin-5-yl)oxy)cyclopentyl)amino)methyl)cyclohexane-1-carbonitrile;
3-(5-(((1S,2S)-2-(((4-methoxycyclohexyl)methyl)amino)cyclopentyl)oxy)-1-oxoisoindolin-2-yl)piperidine-2,6-dione;
3-(5-(((1S,2R)-2-(((4-methoxycyclohexyl)methyl)amino)cyclopentyl)oxy)-1-oxoisoindolin-2-yl)piperidine-2,6-dione;
3-(5-(((1R,2R)-2-(((4-methoxycyclohexyl)methyl)amino)cyclopentyl)oxy)-1-oxoisoindolin-2-yl)piperidine-2,6-dione;
3-(5-(((1R,2S)-2-(((4-methoxycyclohexyl)methyl)amino)cyclopentyl)oxy)-1-oxoisoindolin-2-yl)piperidine-2,6-dione;
3-(5-((2-(((4-methoxycyclohexyl)methyl)amino)cyclopentyl)oxy)-1-oxoisoindolin-2-yl)piperidine-2,6-dione;
3-(5-(((1S,2S)-2-(2-oxa-7-azaspiro[3.5]nonan-7-yl)cyclopentyl)oxy)-1-oxoisoindolin-2-yl)piperidine-2,6-dione;
3-(5-(((1S,2R)-2-(2-oxa-7-azaspiro[3.5]nonan-7-yl)cyclopentyl)oxy)-1-oxoisoindolin-2-yl)piperidine-2,6-dione;
3-(5-(((1R,2R)-2-(2-oxa-7-azaspiro[3.5]nonan-7-yl)cyclopentyl)oxy)-1-oxoisoindolin-2-yl)piperidine-2,6-dione;
3-(5-(((1R,2S)-2-(2-oxa-7-azaspiro[3.5]nonan-7-yl)cyclopentyl)oxy)-1-oxoisoindolin-2-yl)piperidine-2,6-dione;
3-(5-((2-(2-oxa-7-azaspiro[3.5]nonan-7-yl)cyclopentyl)oxy)-1-oxoisoindolin-2-yl)piperidine-2,6-dione;
3-((((1S,2S)-2-((2-(2,6-dioxopiperidin-3-yl)-1-oxoisoindolin-5-yl)oxy)cyclopentyl)(ethyl)amino)methyl)-1-methylcyclobutane-1-carbonitrile;
(1S,3R)-3-((((1S,2S)-2-((2-(2,6-dioxopiperidin-3-yl)-1-oxoisoindolin-5-yl)oxy)cyclopentyl)(ethyl)amino)methyl)-1-methylcyclobutane-1-carbonitrile;
(1R,3S)-3-((((1S,2S)-2-((2-(2,6-dioxopiperidin-3-yl)-1-oxoisoindolin-5-yl)oxy)cyclopentyl)(ethyl)amino)methyl)-1-methylcyclobutane-1-carbonitrile;
trans-3-((((1S,2S)-2-((2-(2,6-dioxopiperidin-3-yl)-1-oxoisoindolin-5-yl)oxy)cyclopentyl)(ethyl)amino)methyl)-1-methylcyclobutane-1-carbonitrile;
cis-3-((((1S,2S)-2-((2-(2,6-dioxopiperidin-3-yl)-1-oxoisoindolin-5-yl)oxy)cyclopentyl)(ethyl)amino)methyl)-1-methylcyclobutane-1-carbonitrile;
3-(5-(((1S,2S)-2-(4-fluoropiperidin-1-yl)cyclopentyl)oxy)-1-oxoisoindolin-2-yl)piperidine-2,6-dione;
3-(5-(((1S,2R)-2-(4-fluoropiperidin-1-yl)cyclopentyl)oxy)-1-oxoisoindolin-2-yl)piperidine-2,6-dione;
3-(5-(((1R,2R)-2-(4-fluoropiperidin-1-yl)cyclopentyl)oxy)-1-oxoisoindolin-2-yl)piperidine-2,6-dione;
3-(5-(((1R,2S)-2-(4-fluoropiperidin-1-yl)cyclopentyl)oxy)-1-oxoisoindolin-2-yl)piperidine-2,6-dione;
3-(5-((2-(4-fluoropiperidin-1-yl)cyclopentyl)oxy)-1-oxoisoindolin-2-yl)piperidine-2,6-dione;
3-(5-(((1S,2S)-2-(1,5-oxazocan-5-yl)cyclopentyl)oxy)-1-oxoisoindolin-2-yl)piperidine-2,6-dione;
3-(5-(((1S,2R)-2-(1,5-oxazocan-5-yl)cyclopentyl)oxy)-1-oxoisoindolin-2-yl)piperidine-2,6-dione;
3-(5-(((1R,2R)-2-(1,5-oxazocan-5-yl)cyclopentyl)oxy)-1-oxoisoindolin-2-yl)piperidine-2,6-dione;
3-(5-(((1R,2S)-2-(1,5-oxazocan-5-yl)cyclopentyl)oxy)-1-oxoisoindolin-2-yl)piperidine-2,6-dione;
3-(5-((2-(1,5-oxazocan-5-yl)cyclopentyl)oxy)-1-oxoisoindolin-2-yl)piperidine-2,6-dione;
3-(5-((2-(3,3-dimethylpiperidin-1-yl)cyclopentyl)oxy)-1-oxoisoindolin-2-yl)piperidine-2,6-dione;
3-(5-(((1R,2S)-2-(3,3-dimethylpiperidin-1-yl)cyclopentyl)oxy)-1-oxoisoindolin-2-yl)piperidine-2,6-dione;
3-(5-(((1R,2R)-2-(3,3-dimethylpiperidin-1-yl)cyclopentyl)oxy)-1-oxoisoindolin-2-yl)piperidine-2,6-dione;
3-(5-(((1S,2R)-2-(3,3-dimethylpiperidin-1-yl)cyclopentyl)oxy)-1-oxoisoindolin-2-yl)piperidine-2,6-dione;
3-(5-(((1S,2S)-2-(3,3-dimethylpiperidin-1-yl)cyclopentyl)oxy)-1-oxoisoindolin-2-yl)piperidine-2,6-dione;
3-(5-(((1S,2S)-2-(ethyl(((1R,4S)-4-methoxycyclohexyl)methyl)amino)cyclopentyl)oxy)-1-oxoisoindolin-2-yl)piperidine-2,6-dione;
3-(5-(((1S,2S)-2-((((1R,4S)-4-methoxycyclohexyl)methyl)(methyl)amino)cyclopentyl)oxy)-1-oxoisoindolin-2-yl)piperidine-2,6-dione;
trans-3-(5-((2-(diethylamino)-4,4-dimethylcyclopentyl)oxy)-1-oxoisoindolin-2-yl)piperidine-2,6-dione;
cis-3-(5-((2-(diethylamino)-4,4-dimethylcyclopentyl)oxy)-1-oxoisoindolin-2-yl)piperidine-2,6-dione;
3-(5-((2-(diethylamino)-4,4-dimethylcyclopentyl)oxy)-1-oxoisoindolin-2-yl)piperidine-2,6-dione;
3-(5-(((1R,2S)-2-(diethylamino)-4,4-dimethylcyclopentyl)oxy)-1-oxoisoindolin-2-yl)piperidine-2,6-dione;
3-(5-(((1R,2R)-2-(diethylamino)-4,4-dimethylcyclopentyl)oxy)-1-oxoisoindolin-2-yl)piperidine-2,6-dione;
3-(5-(((1S,2R)-2-(diethylamino)-4,4-dimethylcyclopentyl)oxy)-1-oxoisoindolin-2-yl)piperidine-2,6-dione;
3-(5-(((1S,2S)-2-(diethylamino)-4,4-dimethylcyclopentyl)oxy)-1-oxoisoindolin-2-yl)piperidine-2,6-dione;
3-(5-(((1S,2S)-2-(((4,4-difluorocyclohexyl)methyl)amino)cyclopentyl)oxy)-1-oxoisoindolin-2-yl)piperidine-2,6-dione;
3-(5-(((1S,2R)-2-(((4,4-difluorocyclohexyl)methyl)amino)cyclopentyl)oxy)-1-oxoisoindolin-2-yl)piperidine-2,6-dione;
3-(5-(((1R,2R)-2-(((4,4-difluorocyclohexyl)methyl)amino)cyclopentyl)oxy)-1-oxoisoindolin-2-yl)piperidine-2,6-dione;
3-(5-(((1R,2S)-2-(((4,4-difluorocyclohexyl)methyl)amino)cyclopentyl)oxy)-1-oxoisoindolin-2-yl)piperidine-2,6-dione;
3-(5-((2-(((4,4-difluorocyclohexyl)methyl)amino)cyclopentyl)oxy)-1-oxoisoindolin-2-yl)piperidine-2,6-dione;
3-(5-(((1S,2S)-2-(((1H-indol-5-yl)methyl)amino)cyclopentyl)oxy)-1-oxoisoindolin-2-yl)piperidine-2,6-dione;
3-(5-(((1S,2R)-2-(((1H-indol-5-yl)methyl)amino)cyclopentyl)oxy)-1-oxoisoindolin-2-yl)piperidine-2,6-dione;
3-(5-(((1R,2R)-2-(((1H-indol-5-yl)methyl)amino)cyclopentyl)oxy)-1-oxoisoindolin-2-yl)piperidine-2,6-dione;
3-(5-(((1R,2S)-2-(((1H-indol-5-yl)methyl)amino)cyclopentyl)oxy)-1-oxoisoindolin-2-yl)piperidine-2,6-dione;
3-(5-((2-(((1H-indol-5-yl)methyl)amino)cyclopentyl)oxy)-1-oxoisoindolin-2-yl)piperidine-2,6-dione;
3-(5-(((1S,2S)-2-(4-(tert-butoxy)piperidin-1-yl)cyclopentyl)oxy)-1-oxoisoindolin-2-yl)piperidine-2,6-dione;
3-(5-(((1S,2R)-2-(4-(tert-butoxy)piperidin-1-yl)cyclopentyl)oxy)-1-oxoisoindolin-2--yl)piperidine-2,6-dione;
3-(5-(((1R,2R)-2-(4-(tert-butoxy)piperidin-1-yl)cyclopentyl)oxy)-1-oxoisoindolin-2-yl)piperidine-2,6-dione;
3-(5-(((1R,2S)-2-(4-(tert-butoxy)piperidin-1-yl)cyclopentyl)oxy)-1-oxoisoindolin-2-yl)piperidine-2,6-dione;
3-(5-((2-(4-(tert-butoxy)piperidin-1-yl)cyclopentyl)oxy)-1-oxoisoindolin-2-yl)piperidine-2,6-dione;
3-(5-((2-(2-oxa-8-azaspiro[4.5]decan-8-yl)cyclopentyl)oxy)-1-oxoisoindolin-2-yl)piperidine-2,6-dione;
3-(5-(((1R,2S)-2-(2-oxa-8-azaspiro[4.5]decan-8-yl)cyclopentyl)oxy)-1-oxoisoindolin-2-yl)piperidine-2,6-dione;
3-(5-(((1R,2R)-2-(2-oxa-8-azaspiro[4.5]decan-8-yl)cyclopentyl)oxy)-1-oxoisoindolin-2-yl)piperidine-2,6-dione;
3-(5-(((1S,2R)-2-(2-oxa-8-azaspiro[4.5]decan-8-yl)cyclopentyl)oxy)-1-oxoisoindolin-2-yl)piperidine-2,6-dione;
3-(5-(((1S,2S)-2-(2-oxa-8-azaspiro[4.5]decan-8-yl)cyclopentyl)oxy)-1-oxoisoindolin-2-yl)piperidine-2,6-dione;
3-(5-(((1S,2S)-2-(3-(2-chlorophenoxy)azetidin-1-yl)cyclopentyl)oxy)-1-oxoisoindolin-2-yl)piperidine-2,6-dione;
3-(5-(((1S,2R)-2-(3-(2-chlorophenoxy)azetidin-1-yl)cyclopentyl)oxy)-1-oxoisoindolin-2-yl)piperidine-2,6-dione;
3-(5-(((1R,2R)-2-(3-(2-chlorophenoxy)azetidin-1-yl)cyclopentyl)oxy)-1-oxoisoindolin-2-yl)piperidine-2,6-dione;
3-(5-(((1R,2S)-2-(3-(2-chlorophenoxy)azetidin-1-yl)cyclopentyl)oxy)-1-oxoisoindolin-2-yl)piperidine-2,6-dione;
3-(5-((2-(3-(2-chlorophenoxy)azetidin-1-yl)cyclopentyl)oxy)-1-oxoisoindolin-2-yl)piperidine-2,6-dione;
3-(5-(((1S,2S)-2-(3-(2-methoxyphenoxy)azetidin-1-yl)cyclopentyl)oxy)-1-oxoisoindolin-2-yl)piperidine-2,6-dione;
3-(5-(((1S,2R)-2-(3-(2-methoxyphenoxy)azetidin-1-yl)cyclopentyl)oxy)-1-oxoisoindolin-2-yl)piperidine-2,6-dione;
3-(5-(((1R,2R)-2-(3-(2-methoxyphenoxy)azetidin-1-yl)cyclopentyl)oxy)-1-oxoisoindolin-2-yl)piperidine-2,6-dione;
3-(5-(((1R,2S)-2-(3-(2-methoxyphenoxy)azetidin-1-yl)cyclopentyl)oxy)-1-oxoisoindolin-2-yl)piperidine-2,6-dione;
3-(5-((2-(3-(2-methoxyphenoxy)azetidin-1-yl)cyclopentyl)oxy)-1-oxoisoindolin-2--yl)piperidine-2,6-dione;
3-(5-((2-(6-azaspiro[3.5]nonan-6-yl)cyclopentyl)oxy)-1-oxoisoindolin-2-yl)piperidine-2,6-dione;
3-(5-(((1S,2R)-2-(6-azaspiro[3.5]nonan-6-yl)cyclopentyl)oxy)-1-oxoisoindolin-2-yl)piperidine-2,6-dione;
3-(5-(((1R,2R)-2-(6-azaspiro[3.5]nonan-6-yl)cyclopentyl)oxy)-1-oxoisoindolin-2-yl)piperidine-2,6-dione;
3-(5-(((1R,2S)-2-(6-azaspiro[3.5]nonan-6-yl)cyclopentyl)oxy)-1-oxoisoindolin-2-yl)piperidine-2,6-dione;
3-(5-(((1S,2S)-2-(6-azaspiro[3.5]nonan-6-yl)cyclopentyl)oxy)-1-oxoisoindolin-2-yl)piperidine-2,6-dione;
3-(5-(((1S,2S)-2-(ethyl(((1S,3R)-3-methoxycyclobutyl)methyl)amino)cyclopentyl)oxy)-1-oxoisoindolin-2-yl)piperidine-2,6-dione;
3-(5-(((1S,2R)-2-(ethyl(((1s,3S)-3-methoxycyclobutyl)methyl)amino)cyclopentyl)oxy)-1-oxoisoindolin-2-yl)piperidine-2,6-dione;
3-(5-(((1R,2R)-2-(ethyl(((1s,3S)-3-methoxycyclobutyl)methyl)amino)cyclopentyl)oxy)-1-oxoisoindolin-2-yl)piperidine-2,6-dione;
3-(5-(((1R,2S)-2-(ethyl(((1s,3R)-3-methoxycyclobutyl)methyl)amino)cyclopentyl)oxy)-1-oxoisoindolin-2-yl)piperidine-2,6-dione;
3-(5-((2-(ethyl(((1s,3s)-3-methoxycyclobutyl)methyl)amino)cyclopentyl)oxy)-1-oxoisoindolin-2-yl)piperidine-2,6-dione;
3-(5-(((1S,2S)-2-(diethylamino)-4,4-dimethylcyclopentyl)oxy)-1-oxoisoindolin-2-yl)piperidine-2,6-dione;
3-(5-(((1S,2R)-2-(diethylamino)-4,4-dimethylcyclopentyl)oxy)-1-oxoisoindolin-2-yl)piperidine-2,6-dione;
3-(5-(((1R,2R)-2-(diethylamino)-4,4-dimethylcyclopentyl)oxy)-1-oxoisoindolin-2-yl)piperidine-2,6-dione;
3-(5-(((1R,2S)-2-(diethylamino)-4,4-dimethylcyclopentyl)oxy)-1-oxoisoindolin-2-yl)piperidine-2,6-dione;
3-(5-((2-(diethylamino)-4,4-dimethylcyclopentyl)oxy)-1-oxoisoindolin-2-yl)piperidine-2,6-dione;
3-(5-(((1S,2S)-2-(ethyl(((1R,3S)-3-methoxycyclobutyl)methyl)amino)cyclopentyl)oxy)-1-oxoisoindolin-2-yl)piperidine-2,6-dione;
3-(5-(((1S,2R)-2-(ethyl(((1r,3R)-3-methoxycyclobutyl)methyl)amino)cyclopentyl)oxy)-1-oxoisoindolin-2-yl)piperidine-2,6-dione;
3-(5-(((1R,2R)-2-(ethyl(((1r,3R)-3-methoxycyclobutyl)methyl)amino)cyclopentyl)oxy)-1-oxoisoindolin-2-yl)piperidine-2,6-dione;
3-(5-(((1R,2S)-2-(ethyl(((1r,3S)-3-methoxycyclobutyl)methyl)amino)cyclopentyl)oxy)-1-oxoisoindolin-2-yl)piperidine-2,6-dione;
3-(5-((2-(ethyl(((1r,3r)-3-methoxycyclobutyl)methyl)amino)cyclopentyl)oxy)-1-oxoisoindolin-2-yl)piperidine-2,6-dione;
3-(5-((2-(8-oxa-2-azaspiro[4.5]decan-2-yl)cyclopentyl)oxy)-1-oxoisoindolin-2-yl)piperidine-2,6-dione;
3-(5-(((1R,2S)-2-(8-oxa-2-azaspiro[4.5]decan-2-yl)cyclopentyl)oxy)-1-oxoisoindolin-2-yl)piperidine-2,6-dione;
3-(5-(((1R,2R)-2-(8-oxa-2-azaspiro[4.5]decan-2-yl)cyclopentyl)oxy)-1-oxoisoindolin-2-yl)piperidine-2,6-dione;
3-(5-(((1S,2R)-2-(8-oxa-2-azaspiro[4.5]decan-2-yl)cyclopentyl)oxy)-1-oxoisoindolin-2-yl)piperidine-2,6-dione;
3-(5-(((1S,2S)-2-(8-oxa-2-azaspiro[4.5]decan-2-yl)cyclopentyl)oxy)-1-oxoisoindolin-2-yl)piperidine-2,6-dione;
3-(1-oxo-5-(((1S,2S)-2-thiomorpholinocyclopentyl)oxy)isoindolin-2-yl)piperidine-2,6-dione;
3-(1-oxo-5-(((1S,2R)-2-thiomorpholinocyclopentyl)oxy)isoindolin-2-yl)piperidine-2,6-dione;
3-(1-oxo-5-(((1R,2R)-2-thiomorpholinocyclopentyl)oxy)isoindolin-2-yl)piperidine-2,6-dione;
3-(1-oxo-5-(((1R,2S)-2-thiomorpholinocyclopentyl)oxy)isoindolin-2-yl)piperidine-2,6-dione;
3-(1-oxo-5-((2-thiomorpholinocyclopentyl)oxy)isoindolin-2-yl)piperidine-2,6-dione;
3-(5-(((1S,2S)-2-(1,4-oxazepan-4-yl)cyclopentyl)oxy)-1-oxoisoindolin-2-yl)piperidine-2,6-dione;
3-(5-(((1S,2R)-2-(1,4-oxazepan-4-yl)cyclopentyl)oxy)-1-oxoisoindolin-2-yl)piperidine-2,6-dione;
3-(5-(((1R,2R)-2-(1,4-oxazepan-4-yl)cyclopentyl)oxy)-1-oxoisoindolin-2-yl)piperidine-2,6-dione;
3-(5-(((1R,2S)-2-(1,4-oxazepan-4-yl)cyclopentyl)oxy)-1-oxoisoindolin-2-yl)piperidine-2,6-dione;
3-(5-((2-(1,4-oxazepan-4-yl)cyclopentyl)oxy)-1-oxoisoindolin-2-yl)piperidine-2,6-dione;
3-(5-(((1S,2S)-2-(4-isopropoxypiperidin-1-yl)cyclopentyl)oxy)-1-oxoisoindolin-2-yl)piperidine-2,6-dione;
3-(5-(((1S,2R)-2-(4-isopropoxypiperidin-1-yl)cyclopentyl)oxy)-1-oxoisoindolin-2-yl)piperidine-2,6-dione;
3-(5-(((1R,2R)-2-(4-isopropoxypiperidin-1-yl)cyclopentyl)oxy)-1-oxoisoindolin-2-yl)piperidine-2,6-dione;
3-(5-(((1R,2S)-2-(4-isopropoxypiperidin-1-yl)cyclopentyl)oxy)-1-oxoisoindolin-2--yl)piperidine-2,6-dione;
3-(5-((2-(4-isopropoxypiperidin-1-yl)cyclopentyl)oxy)-1-oxoisoindolin-2-yl)piperidine-2,6-dione;
3-(5-(((1S,2S)-2-(4-(cyclopropylmethoxy)piperidin-1-yl)cyclopentyl)oxy)-1-oxoisoindolin-2-yl)piperidine-2,6-dione;
3-(5-(((1S,2R)-2-(4-(cyclopropylmethoxy)piperidin-1-yl)cyclopentyl)oxy)-1-oxoisoindolin-2-yl)piperidine-2,6-dione;
3-(5-(((1R,2R)-2-(4-(cyclopropylmethoxy)piperidin-1-yl)cyclopentyl)oxy)-1-oxoisoindolin-2-yl)piperidine-2,6-dione;
3-(5-(((1R,2S)-2-(4-(cyclopropylmethoxy)piperidin-1-yl)cyclopentyl)oxy)-1-oxoisoindolin-2-yl)piperidine-2,6-dione;
3-(5-((2-(4-(cyclopropylmethoxy)piperidin-1-yl)cyclopentyl)oxy)-1-oxoisoindolin-2-yl)piperidine-2,6-dione;
3-(5-(((1S,2S)-2-((3aR,4R,7S,7aS)-octahydro-2H-4,7-epoxyisoindol-2-yl)cyclopentyl)oxy)-1-oxoisoindolin-2-yl)piperidine-2,6-dione;
3-(5-(((1S,2R)-2-((3aR,4R,7S,7aS)-octahydro-2H-4,7-epoxyisoindol-2-yl)cyclopentyl)oxy)-1-oxoisoindolin-2-yl)piperidine-2,6-dione;
3-(5-(((1R,2R)-2-((3aR,4R,7S,7aS)-octahydro-2H-4,7-epoxyisoindol-2-yl)cyclopentyl)oxy)-1-oxoisoindolin-2-yl)piperidine-2,6-dione;
3-(5-(((1R,2S)-2-((3aR,4R,7S,7aS)-octahydro-2H-4,7-epoxyisoindol-2-yl)cyclopentyl)oxy)-1-oxoisoindolin-2-yl)piperidine-2,6-dione;
3-(5-((2-((3aR,4R,7S,7aS)-octahydro-2H-4,7-epoxyisoindol-2-yl)cyclopentyl)oxy)-1-oxoisoindolin-2-yl)piperidine-2,6-dione;
3-(5-(((1S,2S)-2-(4-ethoxypiperidin-1-yl)cyclopentyl)oxy)-1-oxoisoindolin-2-yl)piperidine-2,6-dione;
3-(5-(((1S,2R)-2-(4-ethoxypiperidin-1-yl)cyclopentyl)oxy)-1-oxoisoindolin-2-yl)piperidine-2,6-dione;
3-(5-(((1R,2R)-2-(4-ethoxypiperidin-1-yl)cyclopentyl)oxy)-1-oxoisoindolin-2-yl)piperidine-2,6-dione;
3-(5-(((1R,2S)-2-(4-ethoxypiperidin-1-yl)cyclopentyl)oxy)-1-oxoisoindolin-2-yl)piperidine-2,6-dione;
3-(5-((2-(4-ethoxypiperidin-1-yl)cyclopentyl)oxy)-1-oxoisoindolin-2-yl)piperidine-2,6-dione;
3-(5-(((1S,2S)-2-(7,8-dihydro-1,6-naphthyridin-6(5H)-yl)cyclopentyl)oxy)-1-oxoisoindolin-2-yl)piperidine-2,6-dione;
3-(5-(((1S,2R)-2-(7,8-dihydro-1,6-naphthyridin-6(5H)-yl)cyclopentyl)oxy)-1-oxoisoindolin-2-yl)piperidine-2,6-dione;
3-(5-(((1R,2R)-2-(7,8-dihydro-1,6-naphthyridin-6(5H)-yl)cyclopentyl)oxy)-1-oxoisoindolin-2-yl)piperidine-2,6-dione;
3-(5-(((1R,2S)-2-(7,8-dihydro-1,6-naphthyridin-6(5H)-yl)cyclopentyl)oxy)-1-oxoisoindolin-2-yl)piperidine-2,6-dione;
3-(5-((2-(7,8-dihydro-1,6-naphthyridin-6(5H)-yl)cyclopentyl)oxy)-1-oxoisoindolin-2-yl)piperidine-2,6-dione;
3-(5-(((1S,2S)-2-((((1S,4R)-4-methoxycyclohexyl)methyl)amino)cyclopentyl)oxy)-1-oxoisoindolin-2-yl)piperidine-2,6-dione;
3-(5-(((1S,2R)-2-((((1s,4S)-4-methoxycyclohexyl)methyl)amino)cyclopentyl)oxy)-1-oxoisoindolin-2-yl)piperidine-2,6-dione;
3-(5-(((1R,2R)-2-((((1s,4S)-4-methoxycyclohexyl)methyl)amino)cyclopentyl)oxy)-1-oxoisoindolin-2-yl)piperidine-2,6-dione;
3-(5-(((1R,2S)-2-((((1s,4R)-4-methoxycyclohexyl)methyl)amino)cyclopentyl)oxy)-1-oxoisoindolin-2-yl)piperidine-2,6-dione;
3-(5-((2-((((1s,4s)-4-methoxycyclohexyl)methyl)amino)cyclopentyl)oxy)-1-oxoisoindolin-2-yl)piperidine-2,6-dione;
3-(5-(((1S,2S)-2-((R)-3-methoxypyrrolidin-1-yl)cyclopentyl)oxy)-1-oxoisoindolin-2-yl)piperidine-2,6-dione;
3-(5-(((1S,2R)-2-((R)-3-methoxypyrrolidin-1-yl)cyclopentyl)oxy)-1-oxoisoindolin-2-yl)piperidine-2,6-dione;
3-(5-(((1R,2R)-2-((R)-3-methoxypyrrolidin-1-yl)cyclopentyl)oxy)-1-oxoisoindolin-2-yl)piperidine-2,6-dione;
3-(5-(((1R,2S)-2-((R)-3-methoxypyrrolidin-1-yl)cyclopentyl)oxy)-1-oxoisoindolin-2-yl)piperidine-2,6-dione;
3-(5-((2-((R)-3-methoxypyrrolidin-1-yl)cyclopentyl)oxy)-1-oxoisoindolin-2-yl)piperidine-2,6-dione;
3-(5-(((1S,2S)-2-((S)-3-methoxypyrrolidin-1-yl)cyclopentyl)oxy)-1-oxoisoindolin-2-yl)piperidine-2,6-dione;
3-(5-(((1S,2R)-2-((S)-3-methoxypyrrolidin-1-yl)cyclopentyl)oxy)-1-oxoisoindolin-2-yl)piperidine-2,6-dione;
3-(5-(((1R,2R)-2-((S)-3-methoxypyrrolidin-1-yl)cyclopentyl)oxy)-1-oxoisoindolin-2-yl)piperidine-2,6-dione;
3-(5-(((1R,2S)-2-((S)-3-methoxypyrrolidin-1-yl)cyclopentyl)oxy)-1-oxoisoindolin-2-yl)piperidine-2,6-dione;
3-(5-((2-((S)-3-methoxypyrrolidin-1-yl)cyclopentyl)oxy)-1-oxoisoindolin-2-yl)piperidine-2,6-dione;
3-(5-(((1S,2S)-2-(((3,3-difluorocyclobutyl)methyl)amino)cyclopentyl)oxy)-1-oxoisoindolin-2-yl)piperidine-2,6-dione;
3-(5-(((1S,2R)-2-(((3,3-difluorocyclobutyl)methyl)amino)cyclopentyl)oxy)-1-oxoisoindolin-2-yl)piperidine-2,6-dione;
3-(5-(((1R,2R)-2-(((3,3-difluorocyclobutyl)methyl)amino)cyclopentyl)oxy)-1-oxoisoindolin-2-yl)piperidine-2,6-dione;
3-(5-(((1R,2S)-2-(((3,3-difluorocyclobutyl)methyl)amino)cyclopentyl)oxy)-1-oxoisoindolin-2-yl)piperidine-2,6-dione;
3-(5-((2-(((3,3-difluorocyclobutyl)methyl)amino)cyclopentyl)oxy)-1-oxoisoindolin-2-yl)piperidine-2,6-dione;
3-(5-(((1S,2S)-2-(4-(difluoromethoxy)piperidin-1-yl)cyclopentyl)oxy)-1-oxoisoindolin-2-yl)piperidine-2,6-dione;
3-(5-(((1S,2R)-2-(4-(difluoromethoxy)piperidin-1-yl)cyclopentyl)oxy)-1-oxoisoindolin-2-yl)piperidine-2,6-dione;
3-(5-(((1R,2R)-2-(4-(difluoromethoxy)piperidin-1-yl)cyclopentyl)oxy)-1-oxoisoindolin-2-yl)piperidine-2,6-dione;
3-(5-(((1R,2S)-2-(4-(difluoromethoxy)piperidin-1-yl)cyclopentyl)oxy)-1-oxoisoindolin-2-yl)piperidine-2,6-dione;
3-(5-((2-(4-(difluoromethoxy)piperidin-1-yl)cyclopentyl)oxy)-1-oxoisoindolin-2-yl)piperidine-2,6-dione;
3-(5-(((1S,2S)-2-(1,3-dihydro-2H-pyrrolo[3,4-c]pyridin-2-yl)cyclopentyl)oxy)-1-oxoisoindolin-2-yl)piperidine-2,6-dione;
3-(5-(((1S,2R)-2-(1,3-dihydro-2H-pyrrolo[3,4-c]pyridin-2-yl)cyclopentyl)oxy)-1-oxoisoindolin-2-yl)piperidine-2,6-dione;
3-(5-(((1R,2R)-2-(1,3-dihydro-2H-pyrrolo[3,4-c]pyridin-2-yl)cyclopentyl)oxy)-1-oxoisoindolin-2-yl)piperidine-2,6-dione;
3-(5-(((1R,2S)-2-(1,3-dihydro-2H-pyrrolo[3,4-c]pyridin-2-yl)cyclopentyl)oxy)-1-oxoisoindolin-2-yl)piperidine-2,6-dione;
3-(5-((2-(1,3-dihydro-2H-pyrrolo[3,4-c]pyridin-2-yl)cyclopentyl)oxy)-1-oxoisoindolin-2-yl)piperidine-2,6-dione;
3-(1-oxo-5-(((1S,2S)-2-(propylamino)cyclohexyl)oxy)isoindolin-2-yl)piperidine-2,6-dione;
3-(1-oxo-5-(((1S,2R)-2-(propylamino)cyclohexyl)oxy)isoindolin-2-yl)piperidine-2,6-dione;
3-(1-oxo-5-(((1R,2R)-2-(propylamino)cyclohexyl)oxy)isoindolin-2-yl)piperidine-2,6-dione;
3-(1-oxo-5-(((1R,2S)-2-(propylamino)cyclohexyl)oxy)isoindolin-2-yl)piperidine-2,6-dione;
3-(1-oxo-5-((2-(propylamino)cyclohexyl)oxy)isoindolin-2-yl)piperidine-2,6-dione;
3-(5-(((1S,2S)-2-(dipropylamino)cyclohexyl)oxy)-1-oxoisoindolin-2-yl)piperidine-2,6-dione;
3-(5-(((1S,2R)-2-(dipropylamino)cyclohexyl)oxy)-1-oxoisoindolin-2-yl)piperidine-2,6-dione;
3-(5-(((1R,2R)-2-(dipropylamino)cyclohexyl)oxy)-1-oxoisoindolin-2-yl)piperidine-2,6-dione;
3-(5-(((1R,2S)-2-(dipropylamino)cyclohexyl)oxy)-1-oxoisoindolin-2-yl)piperidine-2,6-dione;
3-(5-((2-(dipropylamino)cyclohexyl)oxy)-1-oxoisoindolin-2-yl)piperidine-2,6-dione;
3-(1-oxo-5-(((1S,2S)-2-((pyridin-4-ylmethyl)amino)cyclohexyl)oxy)isoindolin-2-yl)piperidine-2,6-dione;
3-(1-oxo-5-(((1S,2R)-2-((pyridin-4-ylmethyl)amino)cyclohexyl)oxy)isoindolin-2-yl)piperidine-2,6-dione;
3-(1-oxo-5-(((1R,2R)-2-((pyridin-4-ylmethyl)amino)cyclohexyl)oxy)isoindolin-2-yl)piperidine-2,6-dione;
3-(1-oxo-5-(((1R,2S)-2-((pyridin-4-ylmethyl)amino)cyclohexyl)oxy)isoindolin-2-yl)piperidine-2,6-dione;
3-(1-oxo-5-((2-((pyridin-4-ylmethyl)amino)cyclohexyl)oxy)isoindolin-2-yl)piperidine-2,6-dione;
3-(5-(((1S,2S)-2-(2-oxa-6-azaspiro[3.3]heptan-6-yl)cyclohexyl)oxy)-1-oxoisoindolin-2-yl)piperidine-2,6-dione;
3-(5-(((1S,2R)-2-(2-oxa-6-azaspiro[3.3]heptan-6-yl)cyclohexyl)oxy)-1-oxoisoindolin-2-yl)piperidine-2,6-dione;
3-(5-(((1R,2R)-2-(2-oxa-6-azaspiro[3.3]heptan-6-yl)cyclohexyl)oxy)-1-oxoisoindolin-2-yl)piperidine-2,6-dione;
3-(5-(((1R,2S)-2-(2-oxa-6-azaspiro[3.3]heptan-6-yl)cyclohexyl)oxy)-1-oxoisoindolin-2-yl)piperidine-2,6-dione;
3-(5-((2-(2-oxa-6-azaspiro[3.3]heptan-6-yl)cyclohexyl)oxy)-1-oxoisoindolin-2-yl)piperidine-2,6-dione;
3-(1-oxo-5-(((1S,2S)-2-((pyridin-3-ylmethyl)amino)cyclohexyl)oxy)isoindolin-2-yl)piperidine-2,6-dione;
3-(1-oxo-5-(((1S,2R)-2-((pyridin-3-ylmethyl)amino)cyclohexyl)oxy)isoindolin-2-yl)piperidine-2,6-dione;
3-(1-oxo-5-(((1R,2R)-2-((pyridin-3-ylmethyl)amino)cyclohexyl)oxy)isoindolin-2-yl)piperidine-2,6-dione;
3-(1-oxo-5-(((1R,2S)-2-((pyridin-3-ylmethyl)amino)cyclohexyl)oxy)isoindolin-2-yl)piperidine-2,6-dione;
3-(1-oxo-5-((2-((pyridin-3-ylmethyl)amino)cyclohexyl)oxy)isoindolin-2-yl)piperidine-2,6-dione;
3-(5-(((1S,2S)-2-(((1-ethyl-1H-pyrazol-4-yl)methyl)amino)cyclohexyl)oxy)-1-oxoisoindolin-2-yl)piperidine-2,6-dione;
3-(5-(((1S,2R)-2-(((1-ethyl-1H-pyrazol-4-yl)methyl)amino)cyclohexyl)oxy)-1-oxoisoindolin-2-yl)piperidine-2,6-dione;
3-(5-(((1R,2R)-2-(((1-ethyl-1H-pyrazol-4-yl)methyl)amino)cyclohexyl)oxy)-1-oxoisoindolin-2-yl)piperidine-2,6-dione;
3-(5-(((1R,2S)-2-(((1-ethyl-1H-pyrazol-4-yl)methyl)amino)cyclohexyl)oxy)-1-oxoisoindolin-2-yl)piperidine-2,6-dione;
3-(5-((2-(((1-ethyl-1H-pyrazol-4-yl)methyl)amino)cyclohexyl)oxy)-1-oxoisoindolin-2-yl)piperidine-2,6-dione;
3-(5-(((1S,2S)-2-(((1-isopropyl-1H-pyrazol-4-yl)methyl)amino)cyclohexyl)oxy)-1-oxoisoindolin-2-yl)piperidine-2,6-dione;
3-(5-(((1S,2R)-2-(((1-isopropyl-1H-pyrazol-4-yl)methyl)amino)cyclohexyl)oxy)-1-oxoisoindolin-2-yl)piperidine-2,6-dione;
3-(5-(((1R,2R)-2-(((1-isopropyl-1H-pyrazol-4-yl)methyl)amino)cyclohexyl)oxy)-1-oxoisoindolin-2-yl)piperidine-2,6-dione;
3-(5-(((1R,2S)-2-(((1-isopropyl-1H-pyrazol-4-yl)methyl)amino)cyclohexyl)oxy)-1-oxoisoindolin-2-yl)piperidine-2,6-dione;
3-(5-((2-(((1-isopropyl-1H-pyrazol-4-yl)methyl)amino)cyclohexyl)oxy)-1-oxoisoindolin-2-yl)piperidine-2,6-dione;
3-(5-(((1S,2S)-2-(ethyl(methyl)amino)cyclohexyl)oxy)-1-oxoisoindolin-2-yl)piperidine-2,6-dione;
3-(5-(((1S,2R)-2-(ethyl(methyl)amino)cyclohexyl)oxy)-1-oxoisoindolin-2-yl)piperidine-2,6-dione;
3-(5-(((1R,2R)-2-(ethyl(methyl)amino)cyclohexyl)oxy)-1-oxoisoindolin-2-yl)piperidine-2,6-dione;
3-(5-(((1R,2S)-2-(ethyl(methyl)amino)cyclohexyl)oxy)-1-oxoisoindolin-2-yl)piperidine-2,6-dione;
3-(5-((2-(ethyl(methyl)amino)cyclohexyl)oxy)-1-oxoisoindolin-2-yl)piperidine-2,6-dione;
3-(5-(((1S,2S)-2-(dimethylamino)cyclohexyl)oxy)-1-oxoisoindolin-2-yl)piperidine-2,6-dione;
3-(5-(((1S,2R)-2-(dimethylamino)cyclohexyl)oxy)-1-oxoisoindolin-2-yl)piperidine-2,6-dione;
3-(5-(((1R,2R)-2-(dimethylamino)cyclohexyl)oxy)-1-oxoisoindolin-2-yl)piperidine-2,6-dione;
3-(5-(((1R,2S)-2-(dimethylamino)cyclohexyl)oxy)-1-oxoisoindolin-2-yl)piperidine-2,6-dione;
3-(5-((2-(dimethylamino)cyclohexyl)oxy)-1-oxoisoindolin-2-yl)piperidine-2,6-dione;
3-(5-(((1S,2S)-2-((oxetan-3-ylmethyl)amino)cyclohexyl)oxy)-1-oxoisoindolin-2-yl)piperidine-2,6-dione;
3-(5-(((1S,2R)-2-((oxetan-3-ylmethyl)amino)cyclohexyl)oxy)-1-oxoisoindolin-2-yl)piperidine-2,6-dione;
3-(5-(((1R,2R)-2-((oxetan-3-ylmethyl)amino)cyclohexyl)oxy)-1-oxoisoindolin-2-yl)piperidine-2,6-dione;
3-(5-(((1R,2S)-2-((oxetan-3-ylmethyl)amino)cyclohexyl)oxy)-1-oxoisoindolin-2--yl)piperidine-2,6-dione;
3-(5-((2-((oxetan-3-ylmethyl)amino)cyclohexyl)oxy)-1-oxoisoindolin-2-yl)piperidine-2,6-dione;
3-(5-(((1S,2S)-2-((2-hydroxyethyl)amino)cyclohexyl)oxy)-1-oxoisoindolin-2-yl)piperidine-2,6-dione;
3-(5-(((1S,2R)-2-((2-hydroxyethyl)amino)cyclohexyl)oxy)-1-oxoisoindolin-2-yl)piperidine-2,6-dione;
3-(5-(((1R,2R)-2-((2-hydroxyethyl)amino)cyclohexyl)oxy)-1-oxoisoindolin-2-yl)piperidine-2,6-dione;
3-(5-(((1R,2S)-2-((2-hydroxyethyl)amino)cyclohexyl)oxy)-1-oxoisoindolin-2-yl)piperidine-2,6-dione;
3-(5-((2-((2-hydroxyethyl)amino)cyclohexyl)oxy)-1-oxoisoindolin-2-yl)piperidine-2,6-dione;
3-(1-oxo-5-(((1S,2S)-2-(pyrrolidin-1-yl)cyclohexyl)oxy)isoindolin-2-yl)piperidine-2,6-dione;
3-(1-oxo-5-(((1S,2R)-2-(pyrrolidin-1-yl)cyclohexyl)oxy)isoindolin-2-yl)piperidine-2,6-dione;
3-(1-oxo-5-(((1R,2R)-2-(pyrrolidin-1-yl)cyclohexyl)oxy)isoindolin-2-yl)piperidine-2,6-dione;
3-(1-oxo-5-(((1R,2S)-2-(pyrrolidin-1-yl)cyclohexyl)oxy)isoindolin-2-yl)piperidine-2,6-dione;
3-(1-oxo-5-((2-(pyrrolidin-1-yl)cyclohexyl)oxy)isoindolin-2-yl)piperidine-2,6-dione;
3-(5-(((1S,2S)-2-morpholinocyclohexyl)oxy)-1-oxoisoindolin-2-yl)piperidine-2,6-dione;
3-(5-(((1S,2R)-2-morpholinocyclohexyl)oxy)-1-oxoisoindolin-2-yl)piperidine-2,6-dione;
3-(5-(((1R,2R)-2-morpholinocyclohexyl)oxy)-1-oxoisoindolin-2-yl)piperidine-2,6-dione;
3-(5-(((1R,2S)-2-morpholinocyclohexyl)oxy)-1-oxoisoindolin-2-yl)piperidine-2,6-dione;
3-(5-((2-morpholinocyclohexyl)oxy)-1-oxoisoindolin-2-yl)piperidine-2,6-dione;
3-(1-oxo-5-(((1S,2S)-2-((pyridin-2-ylmethyl)amino)cyclohexyl)oxy)isoindolin-2-yl)piperidine-2,6-dione;
3-(1-oxo-5-(((1S,2R)-2-((pyridin-2-ylmethyl)amino)cyclohexyl)oxy)isoindolin-2-yl)piperidine-2,6-dione;
3-(1-oxo-5-(((1R,2R)-2-((pyridin-2-ylmethyl)amino)cyclohexyl)oxy)isoindolin-2-yl)piperidine-2,6-dione;
3-(1-oxo-5-(((1R,2S)-2-((pyridin-2-ylmethyl)amino)cyclohexyl)oxy)isoindolin-2-yl)piperidine-2,6-dione;
3-(1-oxo-5-((2-((pyridin-2-ylmethyl)amino)cyclohexyl)oxy)isoindolin-2-yl)piperidine-2,6-dione;
3-(5-(((1S,2S)-2-((3-hydroxy-3-methylbutyl)amino)cyclohexyl)oxy)-1-oxoisoindolin-2-yl)piperidine-2,6-dione;
3-(5-(((1S,2R)-2-((3-hydroxy-3-methylbutyl)amino)cyclohexyl)oxy)-1-oxoisoindolin-2-yl)piperidine-2,6-dione;
3-(5-(((1R,2R)-2-((3-hydroxy-3-methylbutyl)amino)cyclohexyl)oxy)-1-oxoisoindolin-2--yl)piperidine-2,6-dione;
3-(5-(((1R,2S)-2-((3-hydroxy-3-methylbutyl)amino)cyclohexyl)oxy)-1-oxoisoindolin-2-yl)piperidine-2,6-dione;
3-(5-((2-((3-hydroxy-3-methylbutyl)amino)cyclohexyl)oxy)-1-oxoisoindolin-2-yl)piperidine-2,6-dione;
3-(5-(((1S,2S)-2-(((3-methyloxetan-3-yl)methyl)amino)cyclohexyl)oxy)-1-oxoisoindolin-2-yl)piperidine-2,6-dione;
3-(5-(((1S,2R)-2-(((3-methyloxetan-3-yl)methyl)amino)cyclohexyl)oxy)-1-oxoisoindolin-2-yl)piperidine-2,6-dione;
3-(5-(((1R,2R)-2-(((3-methyloxetan-3-yl)methyl)amino)cyclohexyl)oxy)-1-oxoisoindolin-2-yl)piperidine-2,6-dione;
3-(5-(((1R,2S)-2-(((3-methyloxetan-3-yl)methyl)amino)cyclohexyl)oxy)-1-oxoisoindolin-2-yl)piperidine-2,6-dione;
3-(5-((2-(((3-methyloxetan-3-yl)methyl)amino)cyclohexyl)oxy)-1-oxoisoindolin-2-yl)piperidine-2,6-dione;
3-(5-(((1S,2S)-2-(4-methoxy-4-methylpiperidin-1-yl)cyclohexyl)oxy)-1-oxoisoindolin-2-yl)piperidine-2,6-dione;
3-(5-(((1S,2R)-2-(4-methoxy-4-methylpiperidin-1-yl)cyclohexyl)oxy)-1-oxoisoindolin-2-yl)piperidine-2,6-dione;
3-(5-(((1R,2R)-2-(4-methoxy-4-methylpiperidin-1-yl)cyclohexyl)oxy)-1-oxoisoindolin-2-yl)piperidine-2,6-dione;
3-(5-(((1R,2S)-2-(4-methoxy-4-methylpiperidin-1-yl)cyclohexyl)oxy)-1-oxoisoindolin-2-yl)piperidine-2,6-dione;
3-(5-((2-(4-methoxy-4-methylpiperidin-1-yl)cyclohexyl)oxy)-1-oxoisoindolin-2-yl)piperidine-2,6-dione;
3-(5-(((1S,2S)-2-(3-methoxyazetidin-1-yl)cyclohexyl)oxy)-1-oxoisoindolin-2-yl)piperidine-2,6-dione;
3-(5-(((1S,2R)-2-(3-methoxyazetidin-1-yl)cyclohexyl)oxy)-1-oxoisoindolin-2-yl)piperidine-2,6-dione;
3-(5-(((1R,2R)-2-(3-methoxyazetidin-1-yl)cyclohexyl)oxy)-1-oxoisoindolin-2-yl)piperidine-2,6-dione;
3-(5-(((1R,2S)-2-(3-methoxyazetidin-1-yl)cyclohexyl)oxy)-1-oxoisoindolin-2-yl)piperidine-2,6-dione;
3-(5-((2-(3-methoxyazetidin-1-yl)cyclohexyl)oxy)-1-oxoisoindolin-2-yl)piperidine-2,6-dione;
3-(5-(((1S,2S)-2-(((6-methylpyridin-2-yl)methyl)amino)cyclohexyl)oxy)-1-oxoisoindolin-2-yl)piperidine-2,6-dione;
3-(5-(((1S,2R)-2-(((6-methylpyridin-2-yl)methyl)amino)cyclohexyl)oxy)-1-oxoisoindolin-2--yl)piperidine-2,6-dione;
3-(5-(((1R,2R)-2-(((6-methylpyridin-2-yl)methyl)amino)cyclohexyl)oxy)-1-oxoisoindolin-2-yl)piperidine-2,6-dione;
3-(5-(((1R,2S)-2-(((6-methylpyridin-2-yl)methyl)amino)cyclohexyl)oxy)-1-oxoisoindolin-2-yl)piperidine-2,6-dione;
3-(5-((2-(((6-methylpyridin-2-yl)methyl)amino)cyclohexyl)oxy)-1-oxoisoindolin-2-yl)piperidine-2,6-dione;
3-(5-(((1S,2S)-2-(((5-methoxypyridin-2-yl)methyl)amino)cyclohexyl)oxy)-1-oxoisoindolin-2-yl)piperidine-2,6-dione;
3-(5-(((1S,2R)-2-(((5-methoxypyridin-2-yl)methyl)amino)cyclohexyl)oxy)-1-oxoisoindolin-2-yl)piperidine-2,6-dione;
3-(5-(((1R,2R)-2-(((5-methoxypyridin-2-yl)methyl)amino)cyclohexyl)oxy)-1-oxoisoindolin-2-yl)piperidine-2,6-dione;
3-(5-(((1R,2S)-2-(((5-methoxypyridin-2-yl)methyl)amino)cyclohexyl)oxy)-1-oxoisoindolin-2-yl)piperidine-2,6-dione;
3-(5-((2-(((5-methoxypyridin-2-yl)methyl)amino)cyclohexyl)oxy)-1-oxoisoindolin-2-yl)piperidine-2,6-dione;
3-(5-(((1S,2S)-2-(((6-methoxypyridin-3-yl)methyl)amino)cyclohexyl)oxy)-1-oxoisoindolin-2-yl)piperidine-2,6-dione;
3-(5-(((1S,2R)-2-(((6-methoxypyridin-3-yl)methyl)amino)cyclohexyl)oxy)-1-oxoisoindolin-2-yl)piperidine-2,6-dione;
3-(5-(((1R,2R)-2-(((6-methoxypyridin-3-yl)methyl)amino)cyclohexyl)oxy)-1-oxoisoindolin-2-yl)piperidine-2,6-dione;
3-(5-(((1R,2S)-2-(((6-methoxypyridin-3-yl)methyl)amino)cyclohexyl)oxy)-1-oxoisoindolin-2-yl)piperidine-2,6-dione;
3-(5-((2-(((6-methoxypyridin-3-yl)methyl)amino)cyclohexyl)oxy)-1-oxoisoindolin-2-yl)piperidine-2,6-dione;
3-(5-(((1S,2S)-2-((2-hydroxy-2-methylpropyl)amino)cyclohexyl)oxy)-1-oxoisoindolin-2-yl)piperidine-2,6-dione;
3-(5-(((1S,2R)-2-((2-hydroxy-2-methylpropyl)amino)cyclohexyl)oxy)-1-oxoisoindolin-2-yl)piperidine-2,6-dione;
3-(5-(((1R,2R)-2-((2-hydroxy-2-methylpropyl)amino)cyclohexyl)oxy)-1-oxoisoindolin-2-yl)piperidine-2,6-dione;
3-(5-(((1R,2S)-2-((2-hydroxy-2-methylpropyl)amino)cyclohexyl)oxy)-1-oxoisoindolin-2-yl)piperidine-2,6-dione;
3-(5-((2-((2-hydroxy-2-methylpropyl)amino)cyclohexyl)oxy)-1-oxoisoindolin-2--yl)piperidine-2,6-dione; 3-((((1S,2S)-2-((2-(2,6-dioxopiperidin-3-yl)-1-oxoisoindolin-5-yl)oxy)cyclohexyl)amino)methyl)-1-methylcyclobutane-1-carbonitrile;
3-((((1R,2S)-2-((2-(2,6-dioxopiperidin-3-yl)-1-oxoisoindolin-5-yl)oxy)cyclohexyl)amino)methyl)-1-methylcyclobutane-1-carbonitrile;
3-((((1R,2R)-2-((2-(2,6-dioxopiperidin-3-yl)-1-oxoisoindolin-5-yl)oxy)cyclohexyl)amino)methyl)-1-methylcyclobutane-1-carbonitrile;
3-((((1S,2R)-2-((2-(2,6-dioxopiperidin-3-yl)-1-oxoisoindolin-5-yl)oxy)cyclohexyl)amino)methyl)-1-methylcyclobutane-1-carbonitrile;
3-(((2-((2-(2,6-dioxopiperidin-3-yl)-1-oxoisoindolin-5-yl)oxy)cyclohexyl)amino)methyl)-1-methylcyclobutane-1-carbonitrile;
(1S,3R)-3-((((1S,2S)-2-((2-(2,6-dioxopiperidin-3-yl)-1-oxoisoindolin-5-yl)oxy)cyclohexyl)amino)methyl)-1-methylcyclobutane-1-carbonitrile;
(1R,3R)-3-((((1R,2S)-2-((2-(2,6-dioxopiperidin-3-yl)-1-oxoisoindolin-5-yl)oxy)cyclohexyl)amino)methyl)-1-methylcyclobutane-1-carbonitrile;
(1R,3R)-3-((((1R,2R)-2-((2-(2,6-dioxopiperidin-3-yl)-1-oxoisoindolin-5-yl)oxy)cyclohexyl)amino)methyl)-1-methylcyclobutane-1-carbonitrile;
(1S,3r)-3-((((1S,2R)-2-((2-(2,6-dioxopiperidin-3-yl)-1-oxoisoindolin-5-yl)oxy)cyclohexyl)amino)methyl)-1-methylcyclobutane-1-carbonitrile;
(1r,3r)-3-(((2-((2-(2,6-dioxopiperidin-3-yl)-1-oxoisoindolin-5-yl)oxy)cyclohexyl)amino)methyl)-1-methylcyclobutane-1-carbonitrile;
(1R,3S)-3-((((1S,2S)-2-((2-(2,6-dioxopiperidin-3-yl)-1-oxoisoindolin-5-yl)oxy)cyclohexyl)amino)methyl)-1-methylcyclobutane-1-carbonitrile;
(1S,3s)-3-((((1R,2S)-2-((2-(2,6-dioxopiperidin-3-yl)-1-oxoisoindolin-5-yl)oxy)cyclohexyl)amino)methyl)-1-methylcyclobutane-1-carbonitrile;
(1S,3s)-3-((((1R,2R)-2-((2-(2,6-dioxopiperidin-3-yl)-1-oxoisoindolin-5-yl)oxy)cyclohexyl)amino)methyl)-1-methylcyclobutane-1-carbonitrile;
(1R,3s)-3-((((1S,2R)-2-((2-(2,6-dioxopiperidin-3-yl)-1-oxoisoindolin-5-yl)oxy)cyclohexyl)amino)methyl)-1-methylcyclobutane-1-carbonitrile;
(1s,3s)-3-(((2-((2-(2,6-dioxopiperidin-3-yl)-1-oxoisoindolin-5-yl)oxy)cyclohexyl)amino)methyl)-1-methylcyclobutane-1-carbonitrile;
3-(1-oxo-5-(((1S,2S)-2-(piperidin-1-yl)cyclohexyl)oxy)isoindolin-2-yl)piperidine-2,6-dione;
3-(1-oxo-5-(((1S,2R)-2-(piperidin-1-yl)cyclohexyl)oxy)isoindolin-2-yl)piperidine-2,6-dione;
3-(1-oxo-5-(((1R,2R)-2-(piperidin-1-yl)cyclohexyl)oxy)isoindolin-2-yl)piperidine-2,6-dione;
3-(1-oxo-5-(((1R,2S)-2-(piperidin-1-yl)cyclohexyl)oxy)isoindolin-2-yl)piperidine-2,6-dione;
3-(1-oxo-5-((2-(piperidin-1-yl)cyclohexyl)oxy)isoindolin-2-yl)piperidine-2,6-dione;
3-(1-oxo-5-(((1S,2S)-2-(((tetrahydro-2H-pyran-4-yl)methyl)amino)cyclohexyl)oxy)isoindolin-2-yl)piperidine-2,6-dione;
3-(1-oxo-5-(((1S,2R)-2-(((tetrahydro-2H-pyran-4-yl)methyl)amino)cyclohexyl)oxy)isoindolin-2-yl)piperidine-2,6-dione;
3-(1-oxo-5-(((1R,2R)-2-(((tetrahydro-2H-pyran-4-yl)methyl)amino)cyclohexyl)oxy)isoindolin-2-yl)piperidine-2,6-dione;
3-(1-oxo-5-(((1R,2S)-2-(((tetrahydro-2H-pyran-4-yl)methyl)amino)cyclohexyl)oxy)isoindolin-2-yl)piperidine-2,6-dione;
3-(1-oxo-5-((2-(((tetrahydro-2H-pyran-4-yl)methyl)amino)cyclohexyl)oxy)isoindolin-2-yl)piperidine-2,6-dione;
3-(5-(((1S,2S)-2-(((3-methoxycyclobutyl)methyl)amino)cyclohexyl)oxy)-1-oxoisoindolin-2-yl)piperidine-2,6-dione;
3-(5-(((1S,2R)-2-(((3-methoxycyclobutyl)methyl)amino)cyclohexyl)oxy)-1-oxoisoindolin-2-yl)piperidine-2,6-dione;
3-(5-(((1R,2R)-2-(((3-methoxycyclobutyl)methyl)amino)cyclohexyl)oxy)-1-oxoisoindolin-2-yl)piperidine-2,6-dione;
3-(5-(((1R,2S)-2-(((3-methoxycyclobutyl)methyl)amino)cyclohexyl)oxy)-1-oxoisoindolin-2-yl)piperidine-2,6-dione;
3-(5-((2-(((3-methoxycyclobutyl)methyl)amino)cyclohexyl)oxy)-1-oxoisoindolin-2-yl)piperidine-2,6-dione;
3-(5-(((1S,2S)-2-((((1S,3R)-3-methoxycyclobutyl)methyl)amino)cyclohexyl)oxy)-1-oxoisoindolin-2-yl)piperidine-2,6-dione;
3-(5-(((1S,2R)-2-((((1s,3S)-3-methoxycyclobutyl)methyl)amino)cyclohexyl)oxy)-1-oxoisoindolin-2-yl)piperidine-2,6-dione;
3-(5-(((1R,2R)-2-((((1s,3S)-3-methoxycyclobutyl)methyl)amino)cyclohexyl)oxy)-1-oxoisoindolin-2-yl)piperidine-2,6-dione;
3-(5-(((1R,2S)-2-((((1s,3R)-3-methoxycyclobutyl)methyl)amino)cyclohexyl)oxy)-1-oxoisoindolin-2-yl)piperidine-2,6-dione;
3-(5-((2-((((1s,3s)-3-methoxycyclobutyl)methyl)amino)cyclohexyl)oxy)-1-oxoisoindolin-2-yl)piperidine-2,6-dione;
3-(5-(((1S,2S)-2-(((cis-3-methoxycyclobutyl)methyl)amino)cyclohexyl)oxy)-1-oxoisoindolin-2-yl)piperidine-2,6-dione;
3-(5-(((1S,2R)-2-(((cis-3-methoxycyclobutyl)methyl)amino)cyclohexyl)oxy)-1-oxoisoindolin-2-yl)piperidine-2,6-dione;
3-(5-(((1R,2R)-2-(((cis-3-methoxycyclobutyl)methyl)amino)cyclohexyl)oxy)-1-oxoisoindolin-2-yl)piperidine-2,6-dione;
3-(5-(((1R,2S)-2-(((cis-3-methoxycyclobutyl)methyl)amino)cyclohexyl)oxy)-1-oxoisoindolin-2-yl)piperidine-2,6-dione;
3-(5-((2-(((cis-3-methoxycyclobutyl)methyl)amino)cyclohexyl)oxy)-1-oxoisoindolin-2-yl)piperidine-2,6-dione;
3-(5-(((1S,2S)-2-((((1R,3S)-3-methoxycyclobutyl)methyl)amino)cyclohexyl)oxy)-1-oxoisoindolin-2-yl)piperidine-2,6-dione;
3-(5-(((1S,2R)-2-((((1s,3S)-3-methoxycyclobutyl)methyl)amino)cyclohexyl)oxy)-1-oxoisoindolin-2-yl)piperidine-2,6-dione;
3-(5-(((1R,2R)-2-((((1s,3S)-3-methoxycyclobutyl)methyl)amino)cyclohexyl)oxy)-1-oxoisoindolin-2-yl)piperidine-2,6-dione;
3-(5-(((1R,2S)-2-((((1s,3R)-3-methoxycyclobutyl)methyl)amino)cyclohexyl)oxy)-1-oxoisoindolin-2-yl)piperidine-2,6-dione;
3-(5-((2-((((1s,3s)-3-methoxycyclobutyl)methyl)amino)cyclohexyl)oxy)-1-oxoisoindolin-2-yl)piperidine-2,6-dione;
3-(5-(((1S,2S)-2-(((trans-3-methoxycyclobutyl)methyl)amino)cyclohexyl)oxy)-1-oxoisoindolin-2-yl)piperidine-2,6-dione;
3-(5-(((1S,2R)-2-(((trans-3-methoxycyclobutyl)methyl)amino)cyclohexyl)oxy)-1-oxoisoindolin-2-yl)piperidine-2,6-dione;
3-(5-(((1R,2R)-2-(((trans-3-methoxycyclobutyl)methyl)amino)cyclohexyl)oxy)-1-oxoisoindolin-2-yl)piperidine-2,6-dione;
3-(5-(((1R,2S)-2-(((trans-3-methoxycyclobutyl)methyl)amino)cyclohexyl)oxy)-1-oxoisoindolin-2-yl)piperidine-2,6-dione;
3-(5-((2-(((trans-3-methoxycyclobutyl)methyl)amino)cyclohexyl)oxy)-1-oxoisoindolin-2-yl)piperidine-2,6-dione;
3-(5-(((1S,2S)-2-((((1r,4S)-4-methoxycyclohexyl)methyl)amino)cyclohexyl)oxy)-1-oxoisoindolin-2-yl)piperidine-2,6-dione;
3-(5-(((1S,2R)-2-((((1r,4R)-4-methoxycyclohexyl)methyl)amino)cyclohexyl)oxy)-1-oxoisoindolin-2-yl)piperidine-2,6-dione;
3-(5-(((1R,2R)-2-((((1r,4R)-4-methoxycyclohexyl)methyl)amino)cyclohexyl)oxy)-1-oxoisoindolin-2-yl)piperidine-2,6-dione;
3-(5-(((1R,2S)-2-((((1r,4S)-4-methoxycyclohexyl)methyl)amino)cyclohexyl)oxy)-1-oxoisoindolin-2-yl)piperidine-2,6-dione;
3-(5-((2-((((1r,4r)-4-methoxycyclohexyl)methyl)amino)cyclohexyl)oxy)-1-oxoisoindolin-2-yl)piperidine-2,6-dione;
3-(5-(((1S,2S)-2-(((4-methyltetrahydro-2H-pyran-4-yl)methyl)amino)cyclohexyl)oxy)-1-oxoisoindolin-2-yl)piperidine-2,6-dione;
3-(5-(((1S,2R)-2-(((4-methyltetrahydro-2H-pyran-4-yl)methyl)amino)cyclohexyl)oxy)-1-oxoisoindolin-2-yl)piperidine-2,6-dione;
3-(5-(((1R,2R)-2-(((4-methyltetrahydro-2H-pyran-4-yl)methyl)amino)cyclohexyl)oxy)-1-oxoisoindolin-2-yl)piperidine-2,6-dione;
3-(5-(((1R,2S)-2-(((4-methyltetrahydro-2H-pyran-4-yl)methyl)amino)cyclohexyl)oxy)-1-oxoisoindolin-2-yl)piperidine-2,6-dione;
3-(5-((2-(((4-methyltetrahydro-2H-pyran-4-yl)methyl)amino)cyclohexyl)oxy)-1-oxoisoindolin-2-yl)piperidine-2,6-dione;
3-(1-oxo-5-(((1S,2S)-2-((pyrimidin-5-ylmethyl)amino)cyclohexyl)oxy)isoindolin-2-yl)piperidine-2,6-dione;
3-(1-oxo-5-(((1S,2R)-2-((pyrimidin-5-ylmethyl)amino)cyclohexyl)oxy)isoindolin-2-yl)piperidine-2,6-dione;
3-(1-oxo-5-(((1R,2R)-2-((pyrimidin-5-ylmethyl)amino)cyclohexyl)oxy)isoindolin-2-yl)piperidine-2,6-dione;
3-(1-oxo-5-(((1R,2S)-2-((pyrimidin-5-ylmethyl)amino)cyclohexyl)oxy)isoindolin-2-yl)piperidine-2,6-dione;
3-(1-oxo-5-((2-((pyrimidin-5-ylmethyl)amino)cyclohexyl)oxy)isoindolin-2-yl)piperidine-2,6-dione;
3-(1-oxo-5-(((1S,2S)-2-((2-(tetrahydro-2H-pyran-4-yl)ethyl)amino)cyclohexyl)oxy)isoindolin-2-yl)piperidine-2,6-dione;
3-(1-oxo-5-(((1S,2R)-2-((2-(tetrahydro-2H-pyran-4-yl)ethyl)amino)cyclohexyl)oxy)isoindolin-2-yl)piperidine-2,6-dione;
3-(1-oxo-5-(((1R,2R)-2-((2-(tetrahydro-2H-pyran-4-yl)ethyl)amino)cyclohexyl)oxy)isoindolin-2-yl)piperidine-2,6-dione;
3-(1-oxo-5-(((1R,2S)-2-((2-(tetrahydro-2H-pyran-4-yl)ethyl)amino)cyclohexyl)oxy)isoindolin-2-yl)piperidine-2,6-dione;
3-(1-oxo-5-((2-((2-(tetrahydro-2H-pyran-4-yl)ethyl)amino)cyclohexyl)oxy)isoindolin-2-yl)piperidine-2,6-dione;
3-(5-(((1S,2S)-2-(2-oxa-7-azaspiro[3.5]nonan-7-yl)cyclohexyl)oxy)-1-oxoisoindolin-2-yl)piperidine-2,6-dione;
3-(5-(((1S,2R)-2-(2-oxa-7-azaspiro[3.5]nonan-7-yl)cyclohexyl)oxy)-1-oxoisoindolin-2-yl)piperidine-2,6-dione;
3-(5-(((1R,2R)-2-(2-oxa-7-azaspiro[3.5]nonan-7-yl)cyclohexyl)oxy)-1-oxoisoindolin-2-yl)piperidine-2,6-dione;
3-(5-(((1R,2S)-2-(2-oxa-7-azaspiro[3.5]nonan-7-yl)cyclohexyl)oxy)-1-oxoisoindolin-2-yl)piperidine-2,6-dione;
3-(5-((2-(2-oxa-7-azaspiro[3.5]nonan-7-yl)cyclohexyl)oxy)-1-oxoisoindolin-2-yl)piperidine-2,6-dione;
3-(5-(((1S,2S)-2-(((4-methoxytetrahydro-2H-pyran-4-yl)methyl)amino)cyclohexyl)oxy)-1-oxoisoindolin-2-yl)piperidine-2,6-dione;
3-(5-(((1S,2R)-2-(((4-methoxytetrahydro-2H-pyran-4-yl)methyl)amino)cyclohexyl)oxy)-1-oxoisoindolin-2-yl)piperidine-2,6-dione;
3-(5-(((1R,2R)-2-(((4-methoxytetrahydro-2H-pyran-4-yl)methyl)amino)cyclohexyl)oxy)-1-oxoisoindolin-2-yl)piperidine-2,6-dione;
3-(5-(((1R,2S)-2-(((4-methoxytetrahydro-2H-pyran-4-yl)methyl)amino)cyclohexyl)oxy)-1-oxoisoindolin-2-yl)piperidine-2,6-dione;
3-(5-((2-(((4-methoxytetrahydro-2H-pyran-4-yl)methyl)amino)cyclohexyl)oxy)-1-oxoisoindolin-2-yl)piperidine-2,6-dione;
3-(5-(((1S,2S)-2-(((2,2-dimethyltetrahydro-2H-pyran-4-yl)methyl)amino)cyclohexyl)oxy)-1-oxoisoindolin-2-yl)piperidine-2,6-dione;
3-(5-(((1S,2R)-2-(((2,2-dimethyltetrahydro-2H-pyran-4-yl)methyl)amino)cyclohexyl)oxy)-1-oxoisoindolin-2-yl)piperidine-2,6-dione;
3-(5-(((1R,2R)-2-(((2,2-dimethyltetrahydro-2H-pyran-4-yl)methyl)amino)cyclohexyl)oxy)-1-oxoisoindolin-2-yl)piperidine-2,6-dione;
3-(5-(((1R,2S)-2-(((2,2-dimethyltetrahydro-2H-pyran-4-yl)methyl)amino)cyclohexyl)oxy)-1-oxoisoindolin-2-yl)piperidine-2,6-dione;
3-(5-((2-(((2,2-dimethyltetrahydro-2H-pyran-4-yl)methyl)amino)cyclohexyl)oxy)-1-oxoisoindolin-2-yl)piperidine-2,6-dione;
3-(5-(((1S,2S)-2-((7-oxaspiro[3.5]nonan-2-yl)amino)cyclohexyl)oxy)-1-oxoisoindolin-2-yl)piperidine-2,6-dione;
3-(5-(((1S,2R)-2-((7-oxaspiro[3.5]nonan-2-yl)amino)cyclohexyl)oxy)-1-oxoisoindolin-2-yl)piperidine-2,6-dione;
3-(5-(((1R,2R)-2-((7-oxaspiro[3.5]nonan-2-yl)amino)cyclohexyl)oxy)-1-oxoisoindolin-2-yl)piperidine-2,6-dione;
3-(5-(((1R,2S)-2-((7-oxaspiro[3.5]nonan-2-yl)amino)cyclohexyl)oxy)-1-oxoisoindolin-2-yl)piperidine-2,6-dione;
3-(5-((2-((7-oxaspiro[3.5]nonan-2-yl)amino)cyclohexyl)oxy)-1-oxoisoindolin-2-yl)piperidine-2,6-dione;
1-((((1S,2S)-2-((2-(2,6-dioxopiperidin-3-yl)-1-oxoisoindolin-5-yl)oxy)cyclohexyl)amino)methyl)cyclobutane-1-carbonitrile;
1-((((1R,2S)-2-((2-(2,6-dioxopiperidin-3-yl)-1-oxoisoindolin-5-yl)oxy)cyclohexyl)amino)methyl)cyclobutane-1-carbonitrile;
1-((((1R,2R)-2-((2-(2,6-dioxopiperidin-3-yl)-1-oxoisoindolin-5-yl)oxy)cyclohexyl)amino)methyl)cyclobutane-1-carbonitrile;
1-((((1S,2R)-2-((2-(2,6-dioxopiperidin-3-yl)-1-oxoisoindolin-5-yl)oxy)cyclohexyl)amino)methyl)cyclobutane-1-carbonitrile;
1-(((2-((2-(2,6-dioxopiperidin-3-yl)-1-oxoisoindolin-5-yl)oxy)cyclohexyl)amino)methyl)cyclobutane-1-carbonitrile;
3-(5-(((1S,2S)-2-(3-(2-chlorophenoxy)azetidin-1-yl)cyclohexyl)oxy)-1-oxoisoindolin-2-yl)piperidine-2,6-dione;
3-(5-(((1S,2R)-2-(3-(2-chlorophenoxy)azetidin-1-yl)cyclohexyl)oxy)-1-oxoisoindolin-2-yl)piperidine-2,6-dione;
3-(5-(((1R,2R)-2-(3-(2-chlorophenoxy)azetidin-1-yl)cyclohexyl)oxy)-1-oxoisoindolin-2-yl)piperidine-2,6-dione;
3-(5-(((1R,2S)-2-(3-(2-chlorophenoxy)azetidin-1-yl)cyclohexyl)oxy)-1-oxoisoindolin-2-yl)piperidine-2,6-dione;
3-(5-((2-(3-(2-chlorophenoxy)azetidin-1-yl)cyclohexyl)oxy)-1-oxoisoindolin-2-yl)piperidine-2,6-dione;
3-(5-(((1S,2S)-2-(3-(2-methoxyphenoxy)azetidin-1-yl)cyclohexyl)oxy)-1-oxoisoindolin-2-yl)piperidine-2,6-dione;
3-(5-(((1S,2R)-2-(3-(2-methoxyphenoxy)azetidin-1-yl)cyclohexyl)oxy)-1-oxoisoindolin-2-yl)piperidine-2,6-dione;
3-(5-(((1R,2R)-2-(3-(2-methoxyphenoxy)azetidin-1-yl)cyclohexyl)oxy)-1-oxoisoindolin-2-yl)piperidine-2,6-dione;
3-(5-(((1R,2S)-2-(3-(2-methoxyphenoxy)azetidin-1-yl)cyclohexyl)oxy)-1-oxoisoindolin-2-yl)piperidine-2,6-dione;
3-(5-((2-(3-(2-methoxyphenoxy)azetidin-1-yl)cyclohexyl)oxy)-1-oxoisoindolin-2-yl)piperidine-2,6-dione;
3-(1-oxo-5-(((1S,2S)-2-((pyrazolo[1,5-a]pyrimidin-6-ylmethyl)amino)cyclohexyl)oxy)isoindolin-2-yl)piperidine-2,6-dione;
3-(1-oxo-5-(((1S,2R)-2-((pyrazolo[1,5-a]pyrimidin-6-ylmethyl)amino)cyclohexyl)oxy)isoindolin-2-yl)piperidine-2,6-dione;
3-(1-oxo-5-(((1R,2R)-2-((pyrazolo[1,5-a]pyrimidin-6-ylmethyl)amino)cyclohexyl)oxy)isoindolin-2-yl)piperidine-2,6-dione;
3-(1-oxo-5-(((1R,2S)-2-((pyrazolo[1,5-a]pyrimidin-6-ylmethyl)amino)cyclohexyl)oxy)isoindolin-2-yl)piperidine-2,6-dione;
3-(1-oxo-5-((2-((pyrazolo[1,5-a]pyrimidin-6-ylmethyl)amino)cyclohexyl)oxy)isoindolin-2-yl)piperidine-2,6-dione;
3-(5-(((1S,2S)-2-((4,4-difluorocyclohexyl)amino)cyclohexyl)oxy)-1-oxoisoindolin-2--yl)piperidine-2,6-dione;
3-(5-(((1S,2R)-2-((4,4-difluorocyclohexyl)amino)cyclohexyl)oxy)-1-oxoisoindolin-2-yl)piperidine-2,6-dione;
3-(5-(((1R,2R)-2-((4,4-difluorocyclohexyl)amino)cyclohexyl)oxy)-1-oxoisoindolin-2-yl)piperidine-2,6-dione;
3-(5-(((1R,2S)-2-((4,4-difluorocyclohexyl)amino)cyclohexyl)oxy)-1-oxoisoindolin-2-yl)piperidine-2,6-dione;
3-(5-((2-((4,4-difluorocyclohexyl)amino)cyclohexyl)oxy)-1-oxoisoindolin-2-yl)piperidine-2,6-dione;
3-(5-(((1S,2S)-2-((2,4-difluorobenzyl)amino)cyclohexyl)oxy)-1-oxoisoindolin-2-yl)piperidine-2,6-dione;
3-(5-(((1S,2R)-2-((2,4-difluorobenzyl)amino)cyclohexyl)oxy)-1-oxoisoindolin-2-yl)piperidine-2,6-dione;
3-(5-(((1R,2R)-2-((2,4-difluorobenzyl)amino)cyclohexyl)oxy)-1-oxoisoindolin-2-yl)piperidine-2,6-dione;
3-(5-(((1R,2S)-2-((2,4-difluorobenzyl)amino)cyclohexyl)oxy)-1-oxoisoindolin-2-yl)piperidine-2,6-dione;
3-(5-((2-((2,4-difluorobenzyl)amino)cyclohexyl)oxy)-1-oxoisoindolin-2-yl)piperidine-2,6-dione;
3-(5-(((1S,2S)-2-((2-methoxycyclopentyl)amino)cyclohexyl)oxy)-1-oxoisoindolin-2-yl)piperidine-2,6-dione;
3-(5-(((1S,2R)-2-((2-methoxycyclopentyl)amino)cyclohexyl)oxy)-1-oxoisoindolin-2-yl)piperidine-2,6-dione;
3-(5-(((1R,2R)-2-((2-methoxycyclopentyl)amino)cyclohexyl)oxy)-1-oxoisoindolin-2-yl)piperidine-2,6-dione;
3-(5-(((1R,2S)-2-((2-methoxycyclopentyl)amino)cyclohexyl)oxy)-1-oxoisoindolin-2-yl)piperidine-2,6-dione;
3-(5-((2-((2-methoxycyclopentyl)amino)cyclohexyl)oxy)-1-oxoisoindolin-2-yl)piperidine-2,6-dione;
3-(5-(((1S,2S)-2-(((1R,2R)-2-methoxycyclopentyl)amino)cyclohexyl)oxy)-1-oxoisoindolin-2-yl)piperidine-2,6-dione;
3-(5-(((1S,2R)-2-(((1R,2R)-2-methoxycyclopentyl)amino)cyclohexyl)oxy)-1-oxoisoindolin-2-yl)piperidine-2,6-dione;
3-(5-(((1R,2R)-2-(((1R,2R)-2-methoxycyclopentyl)amino)cyclohexyl)oxy)-1-oxoisoindolin-2-yl)piperidine-2,6-dione;
3-(5-(((1R,2S)-2-(((1R,2R)-2-methoxycyclopentyl)amino)cyclohexyl)oxy)-1-oxoisoindolin-2-yl)piperidine-2,6-dione;
3-(5-((2-(((1R,2R)-2-methoxycyclopentyl)amino)cyclohexyl)oxy)-1-oxoisoindolin-2-yl)piperidine-2,6-dione;
3-(5-(((1S,2S)-2-(((1S,2S)-2-methoxycyclopentyl)amino)cyclohexyl)oxy)-1-oxoisoindolin-2-yl)piperidine-2,6-dione;
3-(5-(((1S,2R)-2-(((1S,2S)-2-methoxycyclopentyl)amino)cyclohexyl)oxy)-1-oxoisoindolin-2-yl)piperidine-2,6-dione;
3-(5-(((1R,2R)-2-(((1S,2S)-2-methoxycyclopentyl)amino)cyclohexyl)oxy)-1-oxoisoindolin-2-yl)piperidine-2,6-dione;
3-(5-(((1R,2S)-2-(((1S,2S)-2-methoxycyclopentyl)amino)cyclohexyl)oxy)-1-oxoisoindolin-2-yl)piperidine-2,6-dione;
3-(5-((2-(((1S,2S)-2-methoxycyclopentyl)amino)cyclohexyl)oxy)-1-oxoisoindolin-2-yl)piperidine-2,6-dione;
3-((((1S,2S)-2-((2-(2,6-dioxopiperidin-3-yl)-1-oxoisoindolin-5-yl)oxy)cyclohexyl)amino)methyl)bicyclo[1.1.1]pentane-1-carbonitrile;
3-((((1R,2S)-2-((2-(2,6-dioxopiperidin-3-yl)-1-oxoisoindolin-5-yl)oxy)cyclohexyl)amino)methyl)bicyclo[1.1.1]pentane-1-carbonitrile;
3-((((1R,2R)-2-((2-(2,6-dioxopiperidin-3-yl)-1-oxoisoindolin-5-yl)oxy)cyclohexyl)amino)methyl)bicyclo[1.1.1]pentane-1-carbonitrile;
3-((((1S,2R)-2-((2-(2,6-dioxopiperidin-3-yl)-1-oxoisoindolin-5-yl)oxy)cyclohexyl)amino)methyl)bicyclo[1.1.1]pentane-1-carbonitrile; 3-(((2-((2-(2,6-dioxopiperidin-3-yl)-1-oxoisoindolin-5-yl)oxy)cyclohexyl)amino)methyl)bicyclo[1.1.1]pentane-1-carbonitrile;
3-(5-(((1S,2S)-2-(3-(3-fluorophenoxy)azetidin-1-yl)cyclohexyl)oxy)-1-oxoisoindolin-2-yl)piperidine-2,6-dione;
3-(5-(((1S,2R)-2-(3-(3-fluorophenoxy)azetidin-1-yl)cyclohexyl)oxy)-1-oxoisoindolin-2-yl)piperidine-2,6-dione;
3-(5-(((1R,2R)-2-(3-(3-fluorophenoxy)azetidin-1-yl)cyclohexyl)oxy)-1-oxoisoindolin-2-yl)piperidine-2,6-dione;
3-(5-(((1R,2S)-2-(3-(3-fluorophenoxy)azetidin-1-yl)cyclohexyl)oxy)-1-oxoisoindolin-2-yl)piperidine-2,6-dione;
3-(5-((2-(3-(3-fluorophenoxy)azetidin-1-yl)cyclohexyl)oxy)-1-oxoisoindolin-2-yl)piperidine-2,6-dione;
3-(5-(((1S,2S)-2-(((3,3-difluorocyclobutyl)methyl)amino)cyclohexyl)oxy)-1-oxoisoindolin-2-yl)piperidine-2,6-dione;
3-(5-(((1S,2R)-2-(((3,3-difluorocyclobutyl)methyl)amino)cyclohexyl)oxy)-1-oxoisoindolin-2--yl)piperidine-2,6-dione;
3-(5-(((1R,2R)-2-(((3,3-difluorocyclobutyl)methyl)amino)cyclohexyl)oxy)-1-oxoisoindolin-2-yl)piperidine-2,6-dione;
3-(5-(((1R,2S)-2-(((3,3-difluorocyclobutyl)methyl)amino)cyclohexyl)oxy)-1-oxoisoindolin-2-yl)piperidine-2,6-dione;
3-(5-((2-(((3,3-difluorocyclobutyl)methyl)amino)cyclohexyl)oxy)-1-oxoisoindolin-2-yl)piperidine-2,6-dione;
3-(5-(((1S,2S)-2-((((1S,4R)-4-methoxycyclohexyl)methyl)amino)cyclohexyl)oxy)-1-oxoisoindolin-2-yl)piperidine-2,6-dione;
3-(5-(((1S,2R)-2-((((1s,4S)-4-methoxycyclohexyl)methyl)amino)cyclohexyl)oxy)-1-oxoisoindolin-2-yl)piperidine-2,6-dione;
3-(5-(((1R,2R)-2-((((1s,4S)-4-methoxycyclohexyl)methyl)amino)cyclohexyl)oxy)-1-oxoisoindolin-2-yl)piperidine-2,6-dione;
3-(5-(((1R,2S)-2-((((1s,4R)-4-methoxycyclohexyl)methyl)amino)cyclohexyl)oxy)-1-oxoisoindolin-2-yl)piperidine-2,6-dione;
3-(5-((2-((((1s,4s)-4-methoxycyclohexyl)methyl)amino)cyclohexyl)oxy)-1-oxoisoindolin-2-yl)piperidine-2,6-dione;
3-(5-(((1S,2S)-2-(((4-methoxycyclohexyl)methyl)amino)cyclohexyl)oxy)-1-oxoisoindolin-2-yl)piperidine-2,6-dione;
3-(5-(((1S,2S)-2-(((cis-4-methoxycyclohexyl)methyl)amino)cyclohexyl)oxy)-1-oxoisoindolin-2-yl)piperidine-2,6-dione;
3-(5-(((1S,2S)-2-(((trans-4-methoxycyclohexyl)methyl)amino)cyclohexyl)oxy)-1-oxoisoindolin-2-yl)piperidine-2,6-dione;
3-(5-(((1S,2S)-2-(bis(((1R,4S)-4-methoxycyclohexyl)methyl)amino)cyclohexyl)oxy)-1-oxoisoindolin-2-yl)piperidine-2,6-dione;
3-(5-(((1S,2R)-2-(bis(((1r,4R)-4-methoxycyclohexyl)methyl)amino)cyclohexyl)oxy)-1-oxoisoindolin-2-yl)piperidine-2,6-dione;
3-(5-(((1R,2R)-2-(bis(((1r,4R)-4-methoxycyclohexyl)methyl)amino)cyclohexyl)oxy)-1-oxoisoindolin-2-yl)piperidine-2,6-dione;
3-(5-(((1R,2S)-2-(bis(((1r,4S)-4-methoxycyclohexyl)methyl)amino)cyclohexyl)oxy)-1-oxoisoindolin-2-yl)piperidine-2,6-dione;
3-(5-((2-(bis(((1r,4R)-4-methoxycyclohexyl)methyl)amino)cyclohexyl)oxy)-1-oxoisoindolin-2-yl)piperidine-2,6-dione;
4-((((1S,2S)-2-((2-(2,6-dioxopiperidin-3-yl)-1-oxoisoindolin-5-yl)oxy)cyclohexyl)amino)methyl)benzonitrile;
4-((((1R,2S)-2-((2-(2,6-dioxopiperidin-3-yl)-1-oxoisoindolin-5-yl)oxy)cyclohexyl)amino)methyl)benzonitrile;
4-((((1R,2R)-2-((2-(2,6-dioxopiperidin-3-yl)-1-oxoisoindolin-5-yl)oxy)cyclohexyl)amino)methyl)benzonitrile;
4-((((1S,2R)-2-((2-(2,6-dioxopiperidin-3-yl)-1-oxoisoindolin-5-yl)oxy)cyclohexyl)amino)methyl)benzonitrile;
4-(((2-((2-(2,6-dioxopiperidin-3-yl)-1-oxoisoindolin-5-yl)oxy)cyclohexyl)amino)methyl)benzonitrile;
4-((((1S,2S)-2-((2-(2,6-dioxopiperidin-3-yl)-1-oxoisoindolin-5-yl)oxy)cyclohexyl)(methyl)amino)methyl)benzonitrile;
4-((((1R,2S)-2-((2-(2,6-dioxopiperidin-3-yl)-1-oxoisoindolin-5-yl)oxy)cyclohexyl)(methyl)amino)methyl)benzonitrile;
4-((((1R,2R)-2-((2-(2,6-dioxopiperidin-3-yl)-1-oxoisoindolin-5-yl)oxy)cyclohexyl)(methyl)amino)methyl)benzonitrile;
4-((((1S,2R)-2-((2-(2,6-dioxopiperidin-3-yl)-1-oxoisoindolin-5-yl)oxy)cyclohexyl)(methyl)amino)methyl)benzonitrile;
4-(((2-((2-(2,6-dioxopiperidin-3-yl)-1-oxoisoindolin-5-yl)oxy)cyclohexyl)(methyl)amino)methyl)benzonitrile;
3-(5-(((1S,2S)-2-(((3-fluorobicyclo[1.1.1]pentan-1-yl)methyl)amino)cyclohexyl)oxy)-1-oxoisoindolin-2-yl)piperidine-2,6-dione;
3-(5-(((1S,2R)-2-(((3-fluorobicyclo[1.1.1]pentan-1-yl)methyl)amino)cyclohexyl)oxy)-1-oxoisoindolin-2-yl)piperidine-2,6-dione;
3-(5-(((1R,2R)-2-(((3-fluorobicyclo[1.1.1]pentan-1-yl)methyl)amino)cyclohexyl)oxy)-1-oxoisoindolin-2-yl)piperidine-2,6-dione;
3-(5-(((1R,2S)-2-(((3-fluorobicyclo[1.1.1]pentan-1-yl)methyl)amino)cyclohexyl)oxy)-1-oxoisoindolin-2-yl)piperidine-2,6-dione;
3-(5-((2-(((3-fluorobicyclo[1.1.1]pentan-1-yl)methyl)amino)cyclohexyl)oxy)-1-oxoisoindolin-2-yl)piperidine-2,6-dione;
3-(5-((2-(3,3-difluoropyrrolidin-1-yl)cyclohexyl)oxy)-1-oxoisoindolin-2-yl)piperidine-2,6-dione;
3-(5-(((1S,2S)-2-(3,3-difluoropyrrolidin-1-yl)cyclohexyl)oxy)-1-oxoisoindolin-2-yl)piperidine-2,6-dione;
3-(5-(((1S,2R)-2-(3,3-difluoropyrrolidin-1-yl)cyclohexyl)oxy)-1-oxoisoindolin-2-yl)piperidine-2,6-dione;
3-(5-(((1R,2R)-2-(3,3-difluoropyrrolidin-1-yl)cyclohexyl)oxy)-1-oxoisoindolin-2-yl)piperidine-2,6-dione;
3-(5-(((1R,2S)-2-(3,3-difluoropyrrolidin-1-yl)cyclohexyl)oxy)-1-oxoisoindolin-2-yl)piperidine-2,6-dione;
3-(5-(((1S,2S)-2-(ethylamino)cycloheptyl)oxy)-1-oxoisoindolin-2-yl)piperidine-2,6-dione;
3-(5-(((1S,2R)-2-(ethylamino)cycloheptyl)oxy)-1-oxoisoindolin-2-yl)piperidine-2,6-dione;
3-(5-(((1R,2R)-2-(ethylamino)cycloheptyl)oxy)-1-oxoisoindolin-2-yl)piperidine-2,6-dione;
3-(5-(((1R,2S)-2-(ethylamino)cycloheptyl)oxy)-1-oxoisoindolin-2-yl)piperidine-2,6-dione;
3-(5-((2-(ethylamino)cycloheptyl)oxy)-1-oxoisoindolin-2-yl)piperidine-2,6-dione;
3-(5-(((1S,2S)-2-(benzylamino)cycloheptyl)oxy)-1-oxoisoindolin-2-yl)piperidine-2,6-dione;
3-(5-(((1S,2R)-2-(benzylamino)cycloheptyl)oxy)-1-oxoisoindolin-2-yl)piperidine-2,6-dione;
3-(5-(((1R,2R)-2-(benzylamino)cycloheptyl)oxy)-1-oxoisoindolin-2-yl)piperidine-2,6-dione;
3-(5-(((1R,2S)-2-(benzylamino)cycloheptyl)oxy)-1-oxoisoindolin-2-yl)piperidine-2,6-dione;
3-(5-((2-(benzylamino)cycloheptyl)oxy)-1-oxoisoindolin-2-yl)piperidine-2,6-dione;
3-(5-(((1S,2S)-2-(diethylamino)cycloheptyl)oxy)-1-oxoisoindolin-2-yl)piperidine-2,6-dione;
3-(5-(((1S,2R)-2-(diethylamino)cycloheptyl)oxy)-1-oxoisoindolin-2-yl)piperidine-2,6-dione;
3-(5-(((1R,2R)-2-(diethylamino)cycloheptyl)oxy)-1-oxoisoindolin-2-yl)piperidine-2,6-dione;
3-(5-(((1R,2S)-2-(diethylamino)cycloheptyl)oxy)-1-oxoisoindolin-2-yl)piperidine-2,6-dione;
3-(5-((2-(diethylamino)cycloheptyl)oxy)-1-oxoisoindolin-2-yl)piperidine-2,6-dione;
3-(5-(((1S,2S)-2-(4-methoxy-4-methylpiperidin-1-yl)cycloheptyl)oxy)-1-oxoisoindolin-2-yl)piperidine-2,6-dione;
3-(5-(((1S,2R)-2-(4-methoxy-4-methylpiperidin-1-yl)cycloheptyl)oxy)-1-oxoisoindolin-2-yl)piperidine-2,6-dione;
3-(5-(((1R,2R)-2-(4-methoxy-4-methylpiperidin-1-yl)cycloheptyl)oxy)-1-oxoisoindolin-2-yl)piperidine-2,6-dione;
3-(5-(((1R,2S)-2-(4-methoxy-4-methylpiperidin-1-yl)cycloheptyl)oxy)-1-oxoisoindolin-2-yl)piperidine-2,6-dione;
3-(5-((2-(4-methoxy-4-methylpiperidin-1-yl)cycloheptyl)oxy)-1-oxoisoindolin-2-yl)piperidine-2,6-dione;
3-(5-(((1S,2S)-2-(isobutylamino)cycloheptyl)oxy)-1-oxoisoindolin-2-yl)piperidine-2,6-dione;
3-(5-(((1S,2R)-2-(isobutylamino)cycloheptyl)oxy)-1-oxoisoindolin-2-yl)piperidine-2,6-dione;
3-(5-(((1R,2R)-2-(isobutylamino)cycloheptyl)oxy)-1-oxoisoindolin-2-yl)piperidine-2,6-dione;
3-(5-(((1R,2S)-2-(isobutylamino)cycloheptyl)oxy)-1-oxoisoindolin-2-yl)piperidine-2,6-dione;
3-(5-((2-(isobutylamino)cycloheptyl)oxy)-1-oxoisoindolin-2-yl)piperidine-2,6-dione;
3-(1-oxo-5-(((1S,2S)-2-(propylamino)cycloheptyl)oxy)isoindolin-2-yl)piperidine-2,6-dione;
3-(1-oxo-5-(((1S,2R)-2-(propylamino)cycloheptyl)oxy)isoindolin-2-yl)piperidine-2,6-dione;
3-(1-oxo-5-(((1R,2R)-2-(propylamino)cycloheptyl)oxy)isoindolin-2-yl)piperidine-2,6-dione;
3-(1-oxo-5-(((1R,2S)-2-(propylamino)cycloheptyl)oxy)isoindolin-2-yl)piperidine-2,6-dione;
3-(1-oxo-5-((2-(propylamino)cycloheptyl)oxy)isoindolin-2-yl)piperidine-2,6-dione;
3-(5-(((1S,2S)-2-(2-oxa-6-azaspiro[3.3]heptan-6-yl)cycloheptyl)oxy)-1-oxoisoindolin-2--yl)piperidine-2,6-dione;
3-(5-(((1S,2R)-2-(2-oxa-6-azaspiro[3.3]heptan-6-yl)cycloheptyl)oxy)-1-oxoisoindolin-2-yl)piperidine-2,6-dione;
3-(5-(((1R,2R)-2-(2-oxa-6-azaspiro[3.3]heptan-6-yl)cycloheptyl)oxy)-1-oxoisoindolin-2-yl)piperidine-2,6-dione;
3-(5-(((1R,2S)-2-(2-oxa-6-azaspiro[3.3]heptan-6-yl)cycloheptyl)oxy)-1-oxoisoindolin-2-yl)piperidine-2,6-dione;
3-(5-((2-(2-oxa-6-azaspiro[3.3]heptan-6-yl)cycloheptyl)oxy)-1-oxoisoindolin-2-yl)piperidine-2,6-dione;
3-(5-(((1S,2S)-2-((((1R,4S)-4-methoxycyclohexyl)methyl)amino)cycloheptyl)oxy)-1-oxoisoindolin-2-yl)piperidine-2,6-dione;
3-(5-(((1S,2R)-2-((((1r,4R)-4-methoxycyclohexyl)methyl)amino)cycloheptyl)oxy)-1-oxoisoindolin-2-yl)piperidine-2,6-dione;
3-(5-(((1R,2R)-2-((((1r,4R)-4-methoxycyclohexyl)methyl)amino)cycloheptyl)oxy)-1-oxoisoindolin-2-yl)piperidine-2,6-dione;
3-(5-(((1R,2S)-2-((((1r,4S)-4-methoxycyclohexyl)methyl)amino)cycloheptyl)oxy)-1-oxoisoindolin-2-yl)piperidine-2,6-dione;
3-(5-((2-((((1r,4r)-4-methoxycyclohexyl)methyl)amino)cycloheptyl)oxy)-1-oxoisoindolin-2-yl)piperidine-2,6-dione;
3-(1-oxo-5-(((1S,2S)-2-(((tetrahydro-2H-pyran-4-yl)methyl)amino)cycloheptyl)oxy) isoindolin-2-yl)piperidine-2,6-dione;
3-(1-oxo-5-(((1S,2R)-2-(((tetrahydro-2H-pyran-4-yl)methyl)amino)cycloheptyl)oxy)isoindolin-2-yl)piperidine-2,6-dione;
3-(1-oxo-5-(((1R,2R)-2-(((tetrahydro-2H-pyran-4-yl)methyl)amino)cycloheptyl)oxy)isoindolin-2-yl)piperidine-2,6-dione;
3-(1-oxo-5-(((1R,2S)-2-(((tetrahydro-2H-pyran-4-yl)methyl)amino)cycloheptyl)oxy)isoindolin-2-yl)piperidine-2,6-dione;
3-(1-oxo-5-((2-(((tetrahydro-2H-pyran-4-yl)methyl)amino)cycloheptyl)oxy)isoindolin-2-yl)piperidine-2,6-dione;
3-(5-(((1S,2S)-2-(((3-methyloxetan-3-yl)methyl)amino)cycloheptyl)oxy)-1-oxoisoindolin-2-yl)piperidine-2,6-dione;
3-(5-(((1S,2R)-2-(((3-methyloxetan-3-yl)methyl)amino)cycloheptyl)oxy)-1-oxoisoindolin-2-yl)piperidine-2,6-dione;
3-(5-(((1R,2R)-2-(((3-methyloxetan-3-yl)methyl)amino)cycloheptyl)oxy)-1-oxoisoindolin-2-yl)piperidine-2,6-dione;
3-(5-(((1R,2S)-2-(((3-methyloxetan-3-yl)methyl)amino)cycloheptyl)oxy)-1-oxoisoindolin-2--yl)piperidine-2,6-dione;
3-(5-((2-(((3-methyloxetan-3-yl)methyl)amino)cycloheptyl)oxy)-1-oxoisoindolin-2-yl)piperidine-2,6-dione;
3-(5-(((1S,2S)-2-(((3-fluorobicyclo[1.1.1]pentan-1-yl)methyl)amino)cycloheptyl)oxy)-1-oxoisoindolin-2-yl)piperidine-2,6-dione;
3-(5-(((1S,2R)-2-(((3-fluorobicyclo[1.1.1]pentan-1-yl)methyl)amino)cycloheptyl)oxy)-1-oxoisoindolin-2-yl)piperidine-2,6-dione;
3-(5-(((1R,2R)-2-(((3-fluorobicyclo[1.1.1]pentan-1-yl)methyl)amino)cycloheptyl)oxy)-1-oxoisoindolin-2-yl)piperidine-2,6-dione;
3-(5-(((1R,2S)-2-(((3-fluorobicyclo[1.1.1]pentan-1-yl)methyl)amino)cycloheptyl)oxy)-1-oxoisoindolin-2-yl)piperidine-2,6-dione;
3-(5-((2-(((3-fluorobicyclo[1.1.1]pentan-1-yl)methyl)amino)cycloheptyl)oxy)-1-oxoisoindolin-2-yl)piperidine-2,6-dione;
3-(5-(((1S,2S)-2-(3-methoxyazetidin-1-yl)cycloheptyl)oxy)-1-oxoisoindolin-2-yl)piperidine-2,6-dione;
3-(5-(((1S,2R)-2-(3-methoxyazetidin-1-yl)cycloheptyl)oxy)-1-oxoisoindolin-2-yl)piperidine-2,6-dione;
3-(5-(((1R,2R)-2-(3-methoxyazetidin-1-yl)cycloheptyl)oxy)-1-oxoisoindolin-2-yl)piperidine-2,6-dione;
3-(5-(((1R,2S)-2-(3-methoxyazetidin-1-yl)cycloheptyl)oxy)-1-oxoisoindolin-2-yl)piperidine-2,6-dione;
3-(5-((2-(3-methoxyazetidin-1-yl)cycloheptyl)oxy)-1-oxoisoindolin-2-yl)piperidine-2,6-dione;
3-(5-(((1S,2S)-2-(((3,3-difluorocyclobutyl)methyl)amino)cycloheptyl)oxy)-1-oxoisoindolin-2-yl)piperidine-2,6-dione;
3-(5-(((1S,2R)-2-(((3,3-difluorocyclobutyl)methyl)amino)cycloheptyl)oxy)-1-oxoisoindolin-2-yl)piperidine-2,6-dione;
3-(5-(((1R,2R)-2-(((3,3-difluorocyclobutyl)methyl)amino)cycloheptyl)oxy)-1-oxoisoindolin-2-yl)piperidine-2,6-dione;
3-(5-(((1R,2S)-2-(((3,3-difluorocyclobutyl)methyl)amino)cycloheptyl)oxy)-1-oxoisoindolin-2-yl)piperidine-2,6-dione;
3-(5-((2-(((3,3-difluorocyclobutyl)methyl)amino)cycloheptyl)oxy)-1-oxoisoindolin-2-yl)piperidine-2,6-dione;
3-(5-((2-(diethylamino)-3-methylcyclopentyl)oxy)-1-oxoisoindolin-2-yl)piperidine-2,6-dione;
3-(5-(((1S,2S)-2-(diethylamino)-3-methylcyclopentyl)oxy)-1-oxoisoindolin-2-yl)piperidine-2,6-dione;
3-(5-(((1S,2R)-2-(diethylamino)-3-methylcyclopentyl)oxy)-1-oxoisoindolin-2-yl)piperidine-2,6-dione;
3-(5-(((1R,2R)-2-(diethylamino)-3-methylcyclopentyl)oxy)-1-oxoisoindolin-2-yl)piperidine-2,6-dione;
3-(5-(((1R,2S)-2-(diethylamino)-3-methylcyclopentyl)oxy)-1-oxoisoindolin-2-yl)piperidine-2,6-dione;
3-(1-oxo-5-(((1S,2S)-2-(3-(pyridazin-3-yloxy)azetidin-1-yl)cyclohexyl)oxy)isoindolin-2-yl)piperidine-2,6-dione;
3-(1-oxo-5-(((1S,2R)-2-(3-(pyridazin-3-yloxy)azetidin-1-yl)cyclohexyl)oxy)isoindolin-2-yl)piperidine-2,6-dione;
3-(1-oxo-5-(((1R,2R)-2-(3-(pyridazin-3-yloxy)azetidin-1-yl)cyclohexyl)oxy)isoindolin-2-yl)piperidine-2,6-dione;
3-(1-oxo-5-(((1R,2S)-2-(3-(pyridazin-3-yloxy)azetidin-1-yl)cyclohexyl)oxy)isoindolin-2-yl)piperidine-2,6-dione;
3-(1-oxo-5-((2-(3-(pyridazin-3-yloxy)azetidin-1-yl)cyclohexyl)oxy)isoindolin-2-yl)piperidine-2,6-dione;
3-(5-(((1S,2S)-2-(3-isopropoxyazetidin-1-yl)cyclopentyl)oxy)-1-oxoisoindolin-2-yl)piperidine-2,6-dione;
3-(5-(((1S,2R)-2-(3-isopropoxyazetidin-1-yl)cyclopentyl)oxy)-1-oxoisoindolin-2-yl)piperidine-2,6-dione;
3-(5-(((1R,2R)-2-(3-isopropoxyazetidin-1-yl)cyclopentyl)oxy)-1-oxoisoindolin-2-yl)piperidine-2,6-dione;
3-(5-(((1R,2S)-2-(3-isopropoxyazetidin-1-yl)cyclopentyl)oxy)-1-oxoisoindolin-2-yl)piperidine-2,6-dione;
3-(5-((2-(3-isopropoxyazetidin-1-yl)cyclopentyl)oxy)-1-oxoisoindolin-2-yl)piperidine-2,6-dione;
3-(1-oxo-5-(((1S,2S)-2-(3-(2,2,2-trifluoroethoxy)azetidin-1-yl)cyclopentyl)oxy)isoindolin-2-yl)piperidine-2,6-dione;
3-(1-oxo-5-(((1S,2R)-2-(3-(2,2,2-trifluoroethoxy)azetidin-1-yl)cyclopentyl)oxy)isoindolin-2-yl)piperidine-2,6-dione;
3-(1-oxo-5-(((1R,2R)-2-(3-(2,2,2-trifluoroethoxy)azetidin-1-yl)cyclopentyl)oxy)isoindolin-2-yl)piperidine-2,6-dione;
3-(1-oxo-5-(((1R,2S)-2-(3-(2,2,2-trifluoroethoxy)azetidin-1-yl)cyclopentyl)oxy)isoindolin-2-yl)piperidine-2,6-dione;
3-(1-oxo-5-((2-(3-(2,2,2-trifluoroethoxy)azetidin-1-yl)cyclopentyl)oxy)isoindolin-2-yl)piperidine-2,6-dione;
3-(5-(((1S,2S)-2-(3,3-dimethylpiperidin-1-yl)cyclopentyl)oxy)-1-oxoisoindolin-2--yl)piperidine-2,6-dione;
3-(5-(((1S,2R)-2-(3,3-dimethylpiperidin-1-yl)cyclopentyl)oxy)-1-oxoisoindolin-2-yl)piperidine-2,6-dione;
3-(5-(((1R,2S)-2-(3,3-dimethylpiperidin-1-yl)cyclopentyl)oxy)-1-oxoisoindolin-2-yl)piperidine-2,6-dione;
3-(5-(((1R,2R)-2-(3,3-dimethylpiperidin-1-yl)cyclopentyl)oxy)-1-oxoisoindolin-2-yl)piperidine-2,6-dione;
3-(5-((2-(3,3-dimethylpiperidin-1-yl)cyclopentyl)oxy)-1-oxoisoindolin-2-yl)piperidine-2,6-dione;
3-(5-(((1S,2S)-2-(3-isopropoxyazetidin-1-yl)cyclopentyl)oxy)-1-oxoisoindolin-2-yl)piperidine-2,6-dione;
3-(5-(((1S,2R)-2-(3-isopropoxyazetidin-1-yl)cyclopentyl)oxy)-1-oxoisoindolin-2-yl)piperidine-2,6-dione;
3-(5-(((1R,2R)-2-(3-isopropoxyazetidin-1-yl)cyclopentyl)oxy)-1-oxoisoindolin-2-yl)piperidine-2,6-dione;
3-(5-(((1R,2S)-2-(3-isopropoxyazetidin-1-yl)cyclopentyl)oxy)-1-oxoisoindolin-2-yl)piperidine-2,6-dione;
3-(5-((2-(3-isopropoxyazetidin-1-yl)cyclopentyl)oxy)-1-oxoisoindolin-2-yl)piperidine-2,6-dione;
3-(5-(((1S,2S,3S,4R)-3-(3-ethoxyazetidin-1-yl)bicyclo[2.2.1]heptan-2-yl)oxy)-1-oxoisoindolin-2-yl)piperidine-2,6-dione;
3-(5-(((1S,2S,3R,4R)-3-(3-ethoxyazetidin-1-yl)bicyclo[2.2.1]heptan-2-yl)oxy)-1-oxoisoindolin-2-yl)piperidine-2,6-dione;
3-(5-(((1S,2R,3R,4R)-3-(3-ethoxyazetidin-1-yl)bicyclo[2.2.1]heptan-2-yl)oxy)-1-oxoisoindolin-2-yl)piperidine-2,6-dione;
3-(5-(((1S,2R,3S,4R)-3-(3-ethoxyazetidin-1-yl)bicyclo[2.2.1]heptan-2-yl)oxy)-1-oxoisoindolin-2-yl)piperidine-2,6-dione;
3-(5-(((1S,4R)-3-(3-ethoxyazetidin-1-yl)bicyclo[2.2.1]heptan-2-yl)oxy)-1-oxoisoindolin-2-yl)piperidine-2,6-dione;
3-(5-(((1R,2S,3S,4S)-3-(3-ethoxyazetidin-1-yl)bicyclo[2.2.1]heptan-2-yl)oxy)-1-oxoisoindolin-2-yl)piperidine-2,6-dione;
3-(5-(((1R,2S,3R,4S)-3-(3-ethoxyazetidin-1-yl)bicyclo[2.2.1]heptan-2-yl)oxy)-1-oxoisoindolin-2-yl)piperidine-2,6-dione;
3-(5-(((1R,2R,3R,4S)-3-(3-ethoxyazetidin-1-yl)bicyclo[2.2.1]heptan-2-yl)oxy)-1-oxoisoindolin-2-yl)piperidine-2,6-dione;
3-(5-(((1R,2R,3S,4S)-3-(3-ethoxyazetidin-1-yl)bicyclo[2.2.1]heptan-2-yl)oxy)-1-oxoisoindolin-2-yl)piperidine-2,6-dione;
3-(5-(((1R,4S)-3-(3-ethoxyazetidin-1-yl)bicyclo[2.2.1]heptan-2-yl)oxy)-1-oxoisoindolin-2-yl)piperidine-2,6-dione;
3-(1-oxo-5-(((1S,2S)-2-(3-(pyrazin-2-yloxy)azetidin-1-yl)cyclohexyl)oxy)isoindolin-2-yl)piperidine-2,6-dione;
3-(1-oxo-5-(((1S,2R)-2-(3-(pyrazin-2-yloxy)azetidin-1-yl)cyclohexyl)oxy)isoindolin-2-yl)piperidine-2,6-dione;
3-(1-oxo-5-(((1R,2R)-2-(3-(pyrazin-2-yloxy)azetidin-1-yl)cyclohexyl)oxy)isoindolin-2-yl)piperidine-2,6-dione;
3-(1-oxo-5-(((1R,2S)-2-(3-(pyrazin-2-yloxy)azetidin-1-yl)cyclohexyl)oxy)isoindolin-2-yl)piperidine-2,6-dione;
3-(1-oxo-5-((2-(3-(pyrazin-2-yloxy)azetidin-1-yl)cyclohexyl)oxy)isoindolin-2-yl)piperidine-2,6-dione;
3-(5-(((1S,2S)-2-aminocyclopentyl)oxy)-4-fluoro-1-oxoisoindolin-2-yl)piperidine-2,6-dione;
3-(5-(((1S,2R)-2-aminocyclopentyl)oxy)-4-fluoro-1-oxoisoindolin-2-yl)piperidine-2,6-dione;
3-(5-(((1R,2R)-2-aminocyclopentyl)oxy)-4-fluoro-1-oxoisoindolin-2-yl)piperidine-2,6-dione;
3-(5-(((1R,2S)-2-aminocyclopentyl)oxy)-4-fluoro-1-oxoisoindolin-2-yl)piperidine-2,6-dione;
3-(5-((2-aminocyclopentyl)oxy)-4-fluoro-1-oxoisoindolin-2-yl)piperidine-2,6-dione;
3-(5-(((1S,2S)-2-(diethylamino)cyclopentyl)oxy)-4-fluoro-1-oxoisoindolin-2-yl)piperidine-2,6-dione;
3-(5-(((1S,2R)-2-(diethylamino)cyclopentyl)oxy)-4-fluoro-1-oxoisoindolin-2-yl)piperidine-2,6-dione;
3-(5-(((1R,2R)-2-(diethylamino)cyclopentyl)oxy)-4-fluoro-1-oxoisoindolin-2-yl)piperidine-2,6-dione;
3-(5-(((1R,2S)-2-(diethylamino)cyclopentyl)oxy)-4-fluoro-1-oxoisoindolin-2-yl)piperidine-2,6-dione;
3-(5-((2-(diethylamino)cyclopentyl)oxy)-4-fluoro-1-oxoisoindolin-2-yl)piperidine-2,6-dione;
3-(5-(((1S,2S)-2-aminocyclopentyl)oxy)-6-fluoro-1-oxoisoindolin-2-yl)piperidine-2,6-dione;
3-(5-(((1S,2R)-2-aminocyclopentyl)oxy)-6-fluoro-1-oxoisoindolin-2-yl)piperidine-2,6-dione;
3-(5-(((1R,2R)-2-aminocyclopentyl)oxy)-6-fluoro-1-oxoisoindolin-2-yl)piperidine-2,6-dione;
3-(5-(((1R,2S)-2-aminocyclopentyl)oxy)-6-fluoro-1-oxoisoindolin-2-yl)piperidine-2,6-dione;
3-(5-((2-aminocyclopentyl)oxy)-6-fluoro-1-oxoisoindolin-2-yl)piperidine-2,6-dione;
3-(5-(((1S,2S)-2-(diethylamino)cyclopentyl)oxy)-6-fluoro-1-oxoisoindolin-2-yl)piperidine-2,6-dione;
3-(5-(((1S,2R)-2-(diethylamino)cyclopentyl)oxy)-6-fluoro-1-oxoisoindolin-2-yl)piperidine-2,6-dione;
3-(5-(((1R,2R)-2-(diethylamino)cyclopentyl)oxy)-6-fluoro-1-oxoisoindolin-2-yl)piperidine-2,6-dione;
3-(5-(((1R,2S)-2-(diethylamino)cyclopentyl)oxy)-6-fluoro-1-oxoisoindolin-2-yl)piperidine-2,6-dione; and
3-(5-((2-(diethylamino)cyclopentyl)oxy)-6-fluoro-1-oxoisoindolin-2-yl)piperidine-2,6-dione.
   or a pharmaceutically acceptable salt, hydrate, solvate, prodrug, stereoisomer, and tautomer thereof.

Other IKZF2 inhibitors are described in International Publication No. WO2020/012334, which is incorporated by reference in its entirety.

In some embodiments, the IKZF2 inhibitor comprises a compound of Formula (I′),

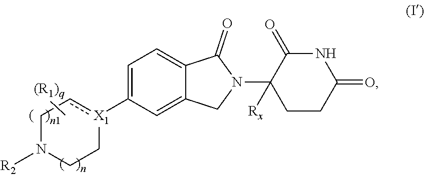

or a pharmaceutically acceptable salt, hydrate, solvate, prodrug, stereoisomer, or tautomer thereof,
wherein:
X₁ is CR₃;
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from 0, N, and S;
R₂ is (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, or (C₃-C₈)cycloalkyl, wherein the alkyl is optionally substituted with one or more R₄; and the aryl, heteroaryl, and cycloalkyl are optionally substituted with one or more R₅, or
R₁ and R₂, when on adjacent atoms, together with the atoms to which they are attached form a 5- or 6-membered heterocycloalkyl ring;
R₃ is H or R₃ is absent when [symbol] is a double bond;
each R₄ is independently selected from —C(O)OR₆, —C(O)NR₆R₆, —NR₆C(O)R₆, halogen, —OH, —NH₂, CN, (C₆-C₁₀)aryl, 5- or 6-membered heteroaryl comprising 1 to 4 heteroatoms selected from 0, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆ are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a=(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a=(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D;
p is 0, 1, or 2;
n is 0, 1, or 2;
n1 is 1 or 2, wherein n+n1≤3; and
q is 0, 1, 2, 3, or 4.

In some embodiments, the compound has a Formula (I),

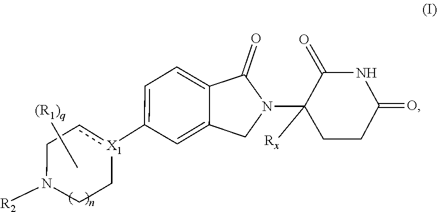

or a pharmaceutically acceptable salt, hydrate, solvate, prodrug, stereoisomer, or tautomer thereof,
wherein:
X₁ is CR₃;
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen;
R₂ is (C₁-C₆)alkyl, (C₆-C₁₀)aryl, 5- or 6-membered heteroaryl comprising 1 to 3 heteroatoms selected from O, N, and S, or (C₃-C₈)cycloalkyl, wherein the alkyl is optionally substituted with one or more R₄; and the aryl, heteroaryl, and cycloalkyl are optionally substituted with one or more R₅;
R₃ is H or R₃ is absent when [symbol] is a double bond;
each R₄ is independently selected from —C(O)OR₆, —C(O)NR₆R_{6′}, —NR₆C(O)R_{6′}, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one or more R₇;
each R₈ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆ are each independently H or (C₁-C₆)alkyl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, CN, (C₆-C₁₀)aryl, 5- or 6-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
Rₓ is H or D;
n is 1 or 2; and
q is 0, 1, 2, 3, or 4.

In some embodiments, n is 1. In some embodiments, n is 2. In some embodiments, q is 0, 1, or 2. In some embodiments, X₁ is CH. In some embodiments, Rₓ is H. In some embodiments, R₂ is (C₆-C₁₀)aryl, or (C₃-C₈)cycloalkyl, wherein the aryl, heteroaryl, and cycloalkyl are optionally substituted with one to three R₅. In some embodiments, R₂ is (C₆-C₁₀)aryl or (C₃-C₈)cycloalkyl. In some embodiments, R₂ is (C₁-C₆)alkyl optionally substituted with one to three R₄.

In some embodiments, the compound has a Formula (Ia), Formula (Ib), Formula (Ic), or Formula (Id):

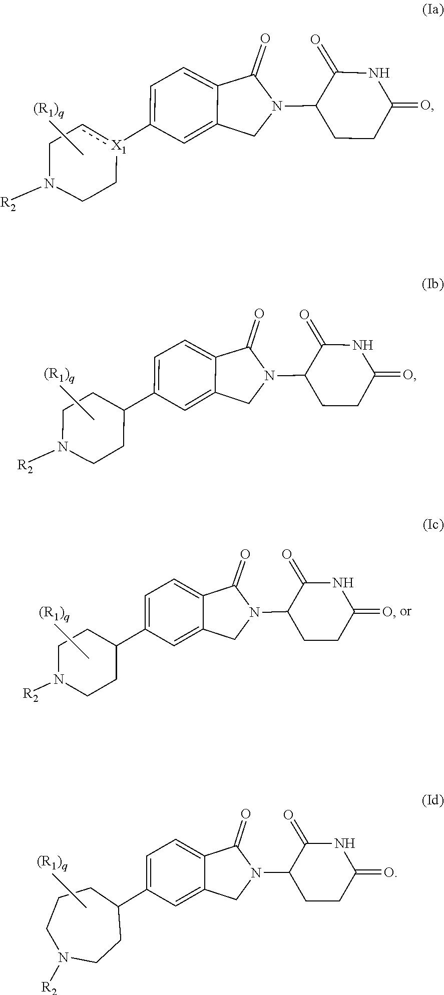

In some embodiments, R₂ is (C₆-C₁₀)aryl or (C₃-C₈)cycloalkyl, wherein the aryl and cycloalkyl are optionally substituted with one to three R₅. In some embodiments, R₂ is (C₆-C₁₀)aryl or (C₃-C₈)cycloalkyl. In some embodiments, R₂ is (C₁-C₆)alkyl optionally substituted with one to three R₄.

In some embodiments, the compound is selected from:
3-(5-(1-ethylpiperidin-4-yl)-1-oxoisoindolin-2-yl)piperidine-2,6-dione;
3-(1-oxo-5-(1-propylpiperidin-4-yl)isoindolin-2-yl)piperidine-2,6-dione;
3-(5-(1-(cyclopropylmethyl)piperidin-4-yl)-1-oxoisoindolin-2-yl)piperidine-2,6-dione;
3-(5-(1-isobutylpiperidin-4-yl)-1-oxoisoindolin-2-yl)piperidine-2,6-dione;
3-(5-(1-(cyclobutylmethyl)piperidin-4-yl)-1-oxoisoindolin-2-yl)piperidine-2,6-dione;
3-(5-(1-(oxazol-2-ylmethyl)piperidin-4-yl)-1-oxoisoindolin-2-yl)piperidine-2,6-dione;
3-(1-oxo-5-(1-(thiazol-2-ylmethyl)piperidin-4-yl)isoindolin-2-yl)piperidine-2,6-dione;
3-(5-(1-(cyclopentylmethyl)piperidin-4-yl)-1-oxoisoindolin-2-yl)piperidine-2,6-dione;
3-(5-(1-((5-chlorothiophen-2-yl)methyl)piperidin-4-yl)-1-oxoisoindolin-2-yl)piperidine-2,6-dione;
3-(5-(1-((2-chlorothiazol-5-yl)methyl)piperidin-4-yl)-1-oxoisoindolin-2-yl)piperidine-2,6-dione;
3-(5-(1-(cyclohexylmethyl)piperidin-4-yl)-1-oxoisoindolin-2-yl)piperidine-2,6-dione;
3-(1-oxo-5-(1-(2-(pyrrolidin-1-yl)ethyl)piperidin-4-yl)isoindolin-2-yl)piperidine-2,6-dione;
3-(1-oxo-5-(1-((tetrahydro-2H-pyran-4-yl)methyl)piperidin-4-yl)isoindolin-2-yl)piperidine-2,6-dione;
3-(1-oxo-5-(1-phenethylpiperidin-4-yl)isoindolin-2-yl)piperidine-2,6-dione;
3-(5-(1-(3-fluorobenzyl)piperidin-4-yl)-1-oxoisoindolin-2-yl)piperidine-2,6-dione;
3-(5-(1-(3-chlorobenzyl)piperidin-4-yl)-1-oxoisoindolin-2-yl)piperidine-2,6-dione;
3-(5-(1-(2-fluorobenzyl)piperidin-4-yl)-1-oxoisoindolin-2-yl)piperidine-2,6-dione;
3-(5-(1-(2-chlorobenzyl)piperidin-4-yl)-1-oxoisoindolin-2-yl)piperidine-2,6-dione;
3-(1-oxo-5-(1-(2-(piperidin-1-yl)ethyl)piperidin-4-yl)isoindolin-2-yl)piperidine-2,6-dione;
3-(5-(1-((3,5-dimethylisoxazol-4-yl)methyl)piperidin-4-yl)-1-oxoisoindolin-2-yl)piperidine-2,6-dione;
3-(5-(1-((1,3-dimethyl-1H-pyrazol-5-yl)methyl)piperidin-4-yl)-1-oxoisoindolin-2-yl)piperidine-2,6-dione;
3-(5-(1-((6-methylpyridin-2-yl)methyl)piperidin-4-yl)-1-oxoisoindolin-2-yl)piperidine-2,6-dione;
3-(5-(1-(3-morpholinopropyl)piperidin-4-yl)-1-oxoisoindolin-2-yl)piperidine-2,6-dione;
3-(5-(1-(2,6-difluorobenzyl)piperidin-4-yl)-1-oxoisoindolin-2-yl)piperidine-2,6-dione;
3-(5-(1-(2,6-dichlorobenzyl)piperidin-4-yl)-1-oxoisoindolin-2-yl)piperidine-2,6-dione;
3-(5-(1-(3,5-difluorobenzyl)piperidin-4-yl)-1-oxoisoindolin-2-yl)piperidine-2,6-dione;
3-(5-(1-(3,5-dibromobenzyl)piperidin-4-yl)-1-oxoisoindolin-2-yl)piperidine-2,6-dione;
3-(5-(1-(3-chloro-5-fluorobenzyl)piperidin-4-yl)-1-oxoisoindolin-2-yl)piperidine-2,6-dione;
3-(5-(1-(2,5-difluorobenzyl)piperidin-4-yl)-1-oxoisoindolin-2-yl)piperidine-2,6-dione;
3-(5-(1-(2,5-dichlorobenzyl)piperidin-4-yl)-1-oxoisoindolin-2-yl)piperidine-2,6-dione;
4-((4-(2-(2,6-dioxopiperidin-3-yl)-1-oxoisoindolin-5-yl)piperidin-1-yl)methyl)benzonitrile;
3-(5-(1-(4-(hydroxymethyl)benzyl)piperidin-4-yl)-1-oxoisoindolin-2-yl)piperidine-2,6-dione;
3-(5-(1-(3,4-dichlorobenzyl)piperidin-4-yl)-1-oxoisoindolin-2-yl)piperidine-2,6-dione;
3-(5-(1-(4-chloro-2-fluorobenzyl)piperidin-4-yl)-1-oxoisoindolin-2-yl)piperidine-2,6-dione;
3-(5-(1-(2-chloro-4-fluorobenzyl)piperidin-4-yl)-1-oxoisoindolin-2-yl)piperidine-2,6-dione; 3-((4-(2-(2,6-dioxopiperidin-3-yl)-1-oxoisoindolin-5-yl)piperidin-1-yl)methyl)benzonitrile;
3-(5-(1-(2,3-difluorobenzyl)piperidin-4-yl)-1-oxoisoindolin-2-yl)piperidine-2,6-dione;
2-((4-(2-(2,6-dioxopiperidin-3-yl)-1-oxoisoindolin-5-yl)piperidin-1-yl)methyl)benzonitrile;
3-(5-(1-(4-methoxybenzyl)piperidin-4-yl)-1-oxoisoindolin-2-yl)piperidine-2,6-dione;
3-(5-(1-(2,5-dimethylbenzyl)piperidin-4-yl)-1-oxoisoindolin-2-yl)piperidine-2,6-dione;
3-(5-(1-(3,4-dimethylbenzyl)piperidin-4-yl)-1-oxoisoindolin-2-yl)piperidine-2,6-dione;
3-(5-(1-(2,4-dimethylbenzyl)piperidin-4-yl)-1-oxoisoindolin-2-yl)piperidine-2,6-dione;
3-(5-(1-((1H-indazol-4-yl)methyl)piperidin-4-yl)-1-oxoisoindolin-2-yl)piperidine-2,6-dione;
3-(5-(1-((1H-benzo[d]imidazol-2-yl)methyl)piperidin-4-yl)-1-oxoisoindolin-2-yl)piperidine-2,6-dione;
3-(5-(1-(4-isopropylbenzyl)piperidin-4-yl)-1-oxoisoindolin-2-yl)piperidine-2,6-dione;
methyl 5-((4-(2-(2,6-dioxopiperidin-3-yl)-1-oxoisoindolin-5-yl)piperidin-1-yl)methyl)furan-2-carboxylate;
3-(5-(1-(naphthalen-2-ylmethyl)piperidin-4-yl)-1-oxoisoindolin-2-yl)piperidine-2,6-dione;
3-(1-oxo-5-(1-(quinolin-2-ylmethyl)piperidin-4-yl)isoindolin-2-yl)piperidine-2,6-dione;
3-(5-(1-(naphthalen-1-ylmethyl)piperidin-4-yl)-1-oxoisoindolin-2-yl)piperidine-2,6-dione;
3-(5-(1-((1-methyl-1H-benzo[d]imidazol-2-yl)methyl)piperidin-4-yl)-1-oxoisoindolin-2-yl)piperidine-2,6-dione;
3-(1-oxo-5-(1-(4-(trifluoromethoxy)benzyl)piperidin-4-yl)isoindolin-2-yl)piperidine-2,6-dione;
3-(5-(1-(4-(1H-pyrrol-1-yl)benzyl)piperidin-4-yl)-1-oxoisoindolin-2-yl)piperidine-2,6-dione;
3-(5-(1-(4-(1H-1,2,4-triazol-1-yl)benzyl)piperidin-4-yl)-1-oxoisoindolin-2-yl)piperidine-2,6-dione;
3-(1-oxo-5-(1-(3-(trifluoromethoxy)benzyl)piperidin-4-yl)isoindolin-2-yl)piperidine-2,6-dione;
3-(1-oxo-5-(1-(2-(trifluoromethoxy)benzyl)piperidin-4-yl)isoindolin-2-yl)piperidine-2,6-dione;
3-(5-(1-benzylpiperidin-4-yl)-1-oxoisoindolin-2-yl)piperidine-2,6-dione;
3-(1-oxo-5-(1-(pyridin-2-ylmethyl)piperidin-4-yl)isoindolin-2-yl)piperidine-2,6-dione;
3-(1-oxo-5-(1-(pyridin-3-ylmethyl)piperidin-4-yl)isoindolin-2-yl)piperidine-2,6-dione;
3-(1-oxo-5-(1-(pyridin-4-ylmethyl)piperidin-4-yl)isoindolin-2-yl)piperidine-2,6-dione;
3-(1-oxo-5-(1-(pyrimidin-5-ylmethyl)piperidin-4-yl)isoindolin-2-yl)piperidine-2,6-dione;
3-(1-oxo-5-(1-(1-phenylethyl)piperidin-4-yl)isoindolin-2-yl)piperidine-2,6-dione;
3-(5-(1-(4-(fluoromethyl)benzyl)piperidin-4-yl)-1-oxoisoindolin-2-yl)piperidine-2,6-dione;
3-(5-(1-(3,4-difluorobenzyl)piperidin-4-yl)-1-oxoisoindolin-2-yl)piperidine-2,6-dione;
2-((4-(2-(2,6-dioxopiperidin-3-yl)-1-oxoisoindolin-5-yl)piperidin-1-yl)methyl)pyrimidine-5-carbonitrile;
3-(5-(1-(4-ethylbenzyl)piperidin-4-yl)-1-oxoisoindolin-2-yl)piperidine-2,6-dione;
3-(5-(1-(2-methoxybenzyl)piperidin-4-yl)-1-oxoisoindolin-2-yl)piperidine-2,6-dione;
3-(5-(1-((2-methoxypyrimidin-5-yl)methyl)piperidin-4-yl)-1-oxoisoindolin-2-yl)piperidine-2,6-dione;
3-(5-(1-(3-fluoro-4-methylbenzyl)piperidin-4-yl)-1-oxoisoindolin-2-yl)piperidine-2,6-dione;
3-(5-(1-(4-(difluoromethyl)benzyl)piperidin-4-yl)-1-oxoisoindolin-2-yl)piperidine-2,6-dione;
4-((4-(2-(2,6-dioxopiperidin-3-yl)-1-oxoisoindolin-5-yl)piperidin-1-yl)methyl)benzamide;
4-((4-(2-(2,6-dioxopiperidin-3-yl)-1-oxoisoindolin-5-yl)piperidin-1-yl)methyl)benzoic acid;
3-(5-(1-(3-(difluoromethyl)benzyl)piperidin-4-yl)-1-oxoisoindolin-2-yl)piperidine-2,6-dione; 3-((4-(2-(2,6-dioxopiperidin-3-yl)-1-oxoisoindolin-5-yl)piperidin-1-yl)methyl)benzoic acid;
3-(1-oxo-5-(1-(4-propylbenzyl)piperidin-4-yl)isoindolin-2-yl)piperidine-2,6-dione;
3-(1-oxo-5-(1-(4-(trifluoromethyl)benzyl)piperidin-4-yl)isoindolin-2-yl)piperidine-2,6-dione;
3-(5-(1-(4-(difluoromethoxy)benzyl)piperidin-4-yl)-1-oxoisoindolin-2-yl)piperidine-2,6-dione;
3-(1-oxo-5-(1-((5-(trifluoromethyl)pyridin-2-yl)methyl)piperidin-4-yl)isoindolin-2--yl)piperidine-2,6-dione;
3-(5-(1-(3-(difluoromethoxy)benzyl)piperidin-4-yl)-1-oxoisoindolin-2-yl)piperidine-2,6-dione;
3-(5-(1-(2-(difluoromethoxy)benzyl)piperidin-4-yl)-1-oxoisoindolin-2-yl)piperidine-2,6-dione;
3-(5-(1-(4-cyclobutylbenzyl)piperidin-4-yl)-1-oxoisoindolin-2-yl)piperidine-2,6-dione;
3-(5-(1-((2,3-dihydrobenzo[b][1,4]dioxin-5-yl)methyl)piperidin-4-yl)-1-oxoisoindolin-2-yl)piperidine-2,6-dione;
3-(5-(1-((2,3-dihydrobenzo[b][1,4]dioxin-6-yl)methyl)piperidin-4-yl)-1-oxoisoindolin-2-yl)piperidine-2,6-dione;
3-(5-(1-(4-(tert-butyl)benzyl)piperidin-4-yl)-1-oxoisoindolin-2-yl)piperidine-2,6-dione;
3-(5-(1-(4-isobutylbenzyl)piperidin-4-yl)-1-oxoisoindolin-2-yl)piperidine-2,6-dione;
N-(4-((4-(2-(2,6-dioxopiperidin-3-yl)-1-oxoisoindolin-5-yl)piperidin-1-yl)methyl)phenyl)acetamide;
3-(5-(1-((2,2-difluorobenzo[d][1,3]dioxol-5-yl)methyl)piperidin-4-yl)-1-oxoisoindolin-2-yl)piperidine-2,6-dione;
3-(5-(1-((3,4-dihydro-2H-benzo[b][1,4]dioxepin-7-yl)methyl)piperidin-4-yl)-1-oxoisoindolin-2-yl)piperidine-2,6-dione;
3-(1-oxo-5-(1-(4-(tert-pentyl)benzyl)piperidin-4-yl)isoindolin-2-yl)piperidine-2,6-dione;
3-(5-(1-([1,1′-biphenyl]-4-ylmethyl)piperidin-4-yl)-1-oxoisoindolin-2-yl)piperidine-2,6-dione;
3-(5-(1-(4-(1H-pyrazol-1-yl)benzyl)piperidin-4-yl)-1-oxoisoindolin-2-yl)piperidine-2,6-dione;
3-(5-(1-(4-(1H-imidazol-1-yl)benzyl)piperidin-4-yl)-1-oxoisoindolin-2-yl)piperidine-2,6-dione;
3-(5-(1-(3-(1H-pyrazol-1-yl)benzyl)piperidin-4-yl)-1-oxoisoindolin-2-yl)piperidine-2,6-dione;
3-(5-(1-(4-cyclohexylbenzyl)piperidin-4-yl)-1-oxoisoindolin-2-yl)piperidine-2,6-dione;
3-(1-oxo-5-(1-(pyrimidin-2-ylmethyl)piperidin-4-yl)isoindolin-2-yl)piperidine-2,6-dione;
3-(5-(1-(4-bromobenzyl)piperidin-4-yl)-1-oxoisoindolin-2-yl)piperidine-2,6-dione;
3-(5-(1-(4-chlorobenzyl)piperidin-4-yl)-1-oxoisoindolin-2-yl)piperidine-2,6-dione;
3-(5-(1-(3,5-dichlorobenzyl)piperidin-4-yl)-1-oxoisoindolin-2-yl)piperidine-2,6-dione
3-(5-(1-(4-chloro-3-fluorobenzyl)piperidin-4-yl)-1-oxoisoindolin-2-yl)piperidine-2,6-dione;
3-(5-(1-(3-chloro-4-fluorobenzyl)piperidin-4-yl)-1-oxoisoindolin-2-yl)piperidine-2,6-dione;
3-(5-(1-(2,4-difluorobenzyl)piperidin-4-yl)-1-oxoisoindolin-2-yl)piperidine-2,6-dione;
3-(5-(1-(3-methoxybenzyl)piperidin-4-yl)-1-oxoisoindolin-2-yl)piperidine-2,6-dione;
3-(5-(1-(benzo[c][1,2,5]oxadiazol-5-ylmethyl)piperidin-4-yl)-1-oxoisoindolin-2--yl)piperidine-2,6-dione;
3-(5-(1-(2-cyclopropylbenzyl)piperidin-4-yl)-1-oxoisoindolin-2-yl)piperidine-2,6-dione;
3-(5-(1-((1,3-dihydroisobenzofuran-5-yl)methyl)piperidin-4-yl)-1-oxoisoindolin-2-yl)piperidine-2,6-dione;
3-(1-oxo-5-(1-(2-(trifluoromethyl)benzyl)piperidin-4-yl)isoindolin-2-yl)piperidine-2,6-dione;
3-(5-(1-(3-isopropoxybenzyl)piperidin-4-yl)-1-oxoisoindolin-2-yl)piperidine-2,6-dione;
3-(1-oxo-5-(1-(4-(thiophen-3-yl)benzyl)piperidin-4-yl)isoindolin-2-yl)piperidine-2,6-dione;
3-(5-(1-(4-cyclopentylbenzyl)piperidin-4-yl)-1-oxoisoindolin-2-yl)piperidine-2,6-dione;
3-(1-oxo-5-(1-(4-(pyrrolidin-1-yl)benzyl)piperidin-4-yl)isoindolin-2-yl)piperidine-2,6-dione;
3-(5-(1-(4-fluorobenzyl)piperidin-4-yl)-1-oxoisoindolin-2-yl)piperidine-2,6-dione;
3-(5-(1-(2,4-dichlorobenzyl)piperidin-4-yl)-1-oxoisoindolin-2-yl)piperidine-2,6-dione;
3-(1-oxo-5-(1-(quinolin-8-ylmethyl)piperidin-4-yl)isoindolin-2-yl)piperidine-2,6-dione;
3-(5-(1-((1-methyl-1H-pyrazol-4-yl)methyl)piperidin-4-yl)-1-oxoisoindolin-2-yl)piperidine-2,6-dione;
3-(5-(1-((1H-pyrazol-4-yl)methyl)piperidin-4-yl)-1-oxoisoindolin-2-yl)piperidine-2,6-dione;
3-(5-(1-((1-methyl-1H-pyrazol-3-yl)methyl)piperidin-4-yl)-1-oxoisoindolin-2-yl)piperidine-2,6-dione;
3-(5-(1-((1H-pyrazol-3-yl)methyl)piperidin-4-yl)-1-oxoisoindolin-2-yl)piperidine-2,6-dione;
3-(5-(1-((1H-pyrrol-3-yl)methyl)piperidin-4-yl)-1-oxoisoindolin-2-yl)piperidine-2,6-dione;
3-(5-(1-((1H-imidazol-5-yl)methyl)piperidin-4-yl)-1-oxoisoindolin-2-yl)piperidine-2,6-dione;
3-(5-(1-((1-ethyl-1H-pyrazol-3-yl)methyl)piperidin-4-yl)-1-oxoisoindolin-2-yl)piperidine-2,6-dione;
3-(5-(1-((2-aminopyrimidin-5-yl)methyl)piperidin-4-yl)-1-oxoisoindolin-2-yl)piperidine-2,6-dione;
3-(5-(1-((6-aminopyridin-3-yl)methyl)piperidin-4-yl)-1-oxoisoindolin-2-yl)piperidine-2,6-dione;
3-(5-(1-((5-amino-1-methyl-1H-pyrazol-4-yl)methyl)piperidin-4-yl)-1-oxoisoindolin-2-yl)piperidine-2,6-dione;
3-(5-(1-((6-methylimidazo[2,1-b]thiazol-5-yl)methyl)piperidin-4-yl)-1-oxoisoindolin-2-yl)piperidine-2,6-dione;
3-(5-(1-(imidazo[1,2-a]pyrazin-3-ylmethyl)piperidin-4-yl)-1-oxoisoindolin-2-yl)piperidine-2,6-dione;
3-(5-(1-([1,2,4]triazolo[1,5-a]pyridin-5-ylmethyl)piperidin-4-yl)-1-oxoisoindolin-2-yl)piperidine-2,6-dione;
3-(1-oxo-5-(1-(pyrazolo[1,5-a]pyridin-4-ylmethyl)piperidin-4-yl)isoindolin-2-yl)piperidine-2,6-dione;
3-(5-(1-((1,4-dimethyl-1H-imidazol-2-yl)methyl)piperidin-4-yl)-1-oxoisoindolin-2--yl)piperidine-2,6-dione;
3-(5-(1-(benzo[d]thiazol-5-ylmethyl)piperidin-4-yl)-1-oxoisoindolin-2-yl)piperidine-2,6-dione;
3-(1-oxo-5-(1-(pyrazolo[1,5-a]pyrimidin-6-ylmethyl)piperidin-4-yl)isoindolin-2-yl)piperidine-2,6-dione;
3-(5-(1-(imidazo[1,2-a]pyrimidin-3-ylmethyl)piperidin-4-yl)-1-oxoisoindolin-2-yl)piperidine-2,6-dione;
3-(5-(1-(imidazo[1,2-a]pyrimidin-2-ylmethyl)piperidin-4-yl)-1-oxoisoindolin-2-yl)piperidine-2,6-dione;
3-(5-(1-((1-cyclobutyl-1H-1,2,3-triazol-4-yl)methyl)piperidin-4-yl)-1-oxoisoindolin-2-yl)piperidine-2,6-dione;
3-(1-oxo-5-(1-((4,5,6,7-tetrahydropyrazolo[1,5-a]pyridin-2-yl)methyl)piperidin-4-yl)isoindolin-2-yl)piperidine-2,6-dione;
3-(5-(1-((1H-indol-2-yl)methyl)piperidin-4-yl)-1-oxoisoindolin-2-yl)piperidine-2,6-dione;
3-(5-(1-((1H-indazol-6-yl)methyl)piperidin-4-yl)-1-oxoisoindolin-2-yl)piperidine-2,6-dione;
3-(5-(1-((1H-pyrrolo[2,3-b]pyridin-3-yl)methyl)piperidin-4-yl)-1-oxoisoindolin-2-yl)piperidine-2,6-dione; 3-((4-(2-(2,6-dioxopiperidin-3-yl)-1-oxoisoindolin-5-yl)piperidin-1-yl)methyl)benzamide;
3-(5-(1-((1H-pyrrolo[2,3-b]pyridin-6-yl)methyl)piperidin-4-yl)-1-oxoisoindolin-2-yl)piperidine-2,6-dione;
3-(5-(1-((3,4-dihydro-2H-benzo[b][1,4]thiazin-6-yl)methyl)piperidin-4-yl)-1-oxoisoindolin-2-yl)piperidine-2,6-dione;
3-(1-oxo-5-(1-((2-(pyrrolidin-1-yl)pyrimidin-5-yl)methyl)piperidin-4-yl)isoindolin-2-yl)piperidine-2,6-dione;
3-(5-(1-((2-(tert-butyl)thiazol-4-yl)methyl)piperidin-4-yl)-1-oxoisoindolin-2-yl)piperidine-2,6-dione;
3-(1-oxo-5-(1-((2-(thiophen-2-yl)thiazol-5-yl)methyl)piperidin-4-yl)isoindolin-2-yl)piperidine-2,6-dione;
3-(5-(1-((5-cyclopropyl-1H-pyrazol-3-yl)methyl)piperidin-4-yl)-1-oxoisoindolin-2-yl)piperidine-2,6-dione;
3-(5-(1-((2-morpholinopyrimidin-5-yl)methyl)piperidin-4-yl)-1-oxoisoindolin-2-yl)piperidine-2,6-dione;
3-(1-oxo-5-(1-((3-phenyl-1H-pyrazol-4-yl)methyl)piperidin-4-yl)isoindolin-2-yl)piperidine-2,6-dione;
3-(5-(1-((6-methyl-1H-indol-3-yl)methyl)piperidin-4-yl)-1-oxoisoindolin-2-yl)piperidine-2,6-dione;
methyl 4-((4-(2-(2,6-dioxopiperidin-3-yl)-1-oxoisoindolin-5-yl)piperidin-1-yl)methyl)-1H-pyrrole-2-carboxylate;
3-(1-oxo-5-(1-((3-(pyridin-3-yl)-1H-pyrazol-4-yl)methyl)piperidin-4-yl)isoindolin-2-yl)piperidine-2,6-dione;
3-(1-oxo-5-(1-((5-(pyridin-2-yl)-1H-pyrazol-3-yl)methyl)piperidin-4-yl)isoindolin-2-yl)piperidine-2,6-dione;
3-(5-(1-(3,5-difluoro-4-hydroxybenzyl)piperidin-4-yl)-1-oxoisoindolin-2-yl)piperidine-2,6-dione;
3-(5-(1-(2-methylbenzyl)piperidin-4-yl)-1-oxoisoindolin-2-yl)piperidine-2,6-dione;
3-(5-(1-(4-methylbenzyl)piperidin-4-yl)-1-oxoisoindolin-2-yl)piperidine-2,6-dione;
3-(5-(1-(3,5-dimethylbenzyl)piperidin-4-yl)-1-oxoisoindolin-2-yl)piperidine-2,6-dione;
3-(5-((2S)-1-benzyl-2-methylpiperidin-4-yl)-1-oxoisoindolin-2-yl)piperidine-2,6-dione;
3-(5-((2R)-1-benzyl-2-methylpiperidin-4-yl)-1-oxoisoindolin-2-yl)piperidine-2,6-dione;
3-(5-(1-benzyl-2-methylpiperidin-4-yl)-1-oxoisoindolin-2-yl)piperidine-2,6-dione;
3-(5-(1-methyl-1,2,3,6-tetrahydropyridin-4-yl)-1-oxoisoindolin-2-yl)piperidine-2,6-dione;
3-(1-oxo-5-(1-((5,6,7,8-tetrahydronaphthalen-1-yl)methyl)piperidin-4-yl)isoindolin-2-yl)piperidine-2,6-dione;
3-(5-(azepan-4-yl)-1-oxoisoindolin-2-yl)piperidine-2,6-dione;
3-(5-((R)-azepan-4-yl)-1-oxoisoindolin-2-yl)piperidine-2,6-dione;
3-(5-((S)-azepan-4-yl)-1-oxoisoindolin-2-yl)piperidine-2,6-dione;
3-(1-oxo-5-(1-((1,2,3,4-tetrahydronaphthalen-1-yl)methyl)piperidin-4-yl)isoindolin-2-yl)piperidine-2,6-dione;
methyl 2-(4-(2-(2,6-dioxopiperidin-3-yl)-1-oxoisoindolin-5-yl)piperidin-1-yl)acetate;
3-(1-oxo-5-(1-phenylpiperidin-4-yl)isoindolin-2-yl)piperidine-2,6-dione;
3-(5-(1-(3-methylbenzyl)piperidin-4-yl)-1-oxoisoindolin-2-yl)piperidine-2,6-dione;
3-(5-(1-(2,6-dimethylbenzyl)piperidin-4-yl)-1-oxoisoindolin-2-yl)piperidine-2,6-dione;
3-(1-oxo-5-(1-((5,6,7,8-tetrahydronaphthalen-2-yl)methyl)piperidin-4-yl)isoindolin-2-yl)piperidine-2,6-dione;
ethyl 2-(4-(2-(2,6-dioxopiperidin-3-yl)-1-oxoisoindolin-5-yl)piperidin-1-yl)acetate;
tert-butyl 2-(4-(2-(2,6-dioxopiperidin-3-yl)-1-oxoisoindolin-5-yl)piperidin-1-yl)acetate;
2-(4-(2-(2,6-dioxopiperidin-3-yl)-1-oxoisoindolin-5-yl)piperidin-1-yl)acetic acid;
3-(1-oxo-5-(1-(3,3,3-trifluoropropyl)piperidin-4-yl)isoindolin-2-yl)piperidine-2,6-dione;
2-(4-(2-(2,6-dioxopiperidin-3-yl)-1-oxoisoindolin-5-yl)piperidin-1-yl)-N-phenylacetamide;
3-(5-(1-(3-fluoropropyl)piperidin-4-yl)-1-oxoisoindolin-2-yl)piperidine-2,6-dione;
tert-butyl 4-((4-(2-(2,6-dioxopiperidin-3-yl)-1-oxoisoindolin-5-yl)piperidin-1-yl)methyl)benzoate;
3-(5-(1-benzyl-3,3-dimethylpiperidin-4-yl)-1-oxoisoindolin-2-yl)piperidine-2,6-dione;
3-(5-(1-benzyl-3-methylpiperidin-4-yl)-1-oxoisoindolin-2-yl)piperidine-2,6-dione;
3-(5-(1-(2-hydroxy-1-phenylethyl)piperidin-4-yl)-1-oxoisoindolin-2-yl)piperidine-2,6-dione;
3-(5-((S)-1-benzylazepan-4-yl)-1-oxoisoindolin-2-yl)piperidine-2,6-dione;
3-(5-(1-benzyl-2,5-dihydro-1H-pyrrol-3-yl)-1-oxoisoindolin-2-yl)piperidine-2,6-dione;
3-(5-(1-benzyl-2-oxopiperidin-4-yl)-1-oxoisoindolin-2-yl)piperidine-2,6-dione;
3-(5-(1-benzyl-1,2,3,4-tetrahydroquinolin-4-yl)-1-oxoisoindolin-2-yl)piperidine-2,6-dione;
3-(5-(1-((1-benzyl-1H-tetrazol-5-yl)methyl)piperidin-4-yl)-1-oxoisoindolin-2-yl)piperidine-2,6-dione;
3-(1-oxo-5-(1-((5-phenyl-1,3,4-oxadiazol-2-yl)methyl)piperidin-4-yl)isoindolin-2-yl)piperidine-2,6-dione;
3-(5-(1-(benzo[d]thiazol-2-ylmethyl)piperidin-4-yl)-1-oxoisoindolin-2-yl)piperidine-2,6-dione;
3-(1-oxo-5-(1-((3-(pyridin-2-yl)-1H-pyrazol-5-yl)methyl)piperidin-4-yl)isoindolin-2-yl)piperidine-2,6-dione;
3-(5-(1-((R)-2-hydroxy-1-phenylethyl)piperidin-4-yl)-1-oxoisoindolin-2-yl)piperidine-2,6-dione;
3-(5-(1-((1-methyl-1H-indazol-3-yl)methyl)piperidin-4-yl)-1-oxoisoindolin-2-yl)piperidine-2,6-dione;
3-(5-(1-((1,2,4-oxadiazol-3-yl)methyl)piperidin-4-yl)-1-oxoisoindolin-2-yl)piperidine-2,6-dione;
3-(5-(1-(4-hydroxy-3-((4-methylpiperazin-1-yl)methyl)benzyl)piperidin-4-yl)-1-oxoisoindolin-2-yl)piperidine-2,6-dione; 2-(4-((4-(2-(2,6-dioxopiperidin-3-yl)-1-oxoisoindolin-5-yl)piperidin-1-yl)methyl)phenyl)acetonitrile;
3-(5-(1-((7-hydroxy-2-methylpyrazolo[1,5-a]pyrimidin-5-yl)methyl)piperidin-4-yl)-1-oxoisoindolin-2-yl)piperidine-2,6-dione;
3-(5-(1-(2,2-difluoro-1-phenylethyl)piperidin-4-yl)-1-oxoisoindolin-2-yl)piperidine-2,6-dione;
3-(5-(1-((3-fluorobicyclo[1.1.1]pentan-1-yl)methyl)piperidin-4-yl)-1-oxoisoindolin-2-yl)piperidine-2,6-dione;
3-(5-(1-(2-fluoro-1-phenylethyl)piperidin-4-yl)-1-oxoisoindolin-2-yl)piperidine-2,6-dione;
3-(1-oxo-5-(1-((4-oxo-3,4-dihydrothieno[3,2-d]pyrimidin-2-yl)methyl)piperidin-4-yl)isoindolin-2-yl)piperidine-2,6-dione;
3-(1-oxo-5-(1-(quinolin-4-ylmethyl)piperidin-4-yl)isoindolin-2-yl)piperidine-2,6-dione;
3-(5-(1-(3,5-bis(trifluoromethyl)benzyl)piperidin-4-yl)-1-oxoisoindolin-2-yl)piperidine-2,6-dione;
6-((4-(2-(2,6-dioxopiperidin-3-yl)-1-oxoisoindolin-5-yl)piperidin-1-yl)methyl)picolinonitrile;
2-(4-((4-(2-(2,6-dioxopiperidin-3-yl)-1-oxoisoindolin-5-yl)piperidin-1-yl)methyl)phenoxy)acetonitrile;
3-(5-(1-((1H-indazol-5-yl)methyl)piperidin-4-yl)-1-oxoisoindolin-2-yl)piperidine-2,6-dione;
3-(5-(1-(2,2-difluoroethyl)piperidin-4-yl)-1-oxoisoindolin-2-yl)piperidine-2,6-dione;
3-(5-(1-((7-methyl-4-oxo-4H-pyrido[1,2-a]pyrimidin-2-yl)methyl)piperidin-4-yl)-1-oxoisoindolin-2-yl)piperidine-2,6-dione;
benzyl 4-(2-(2,6-dioxopiperidin-3-yl)-1-oxoisoindolin-5-yl)piperidine-1-carboxylate;
3-(1-oxo-5-(1-(2-phenylacetyl)piperidin-4-yl)isoindolin-2-yl)piperidine-2,6-dione;
3-(1-oxo-5-(1-(2,2,2-trifluoro-1-phenylethyl)piperidin-4-yl)isoindolin-2-yl)piperidine-2,6-dione;
3-(5-(1-(4-(5-methylbenzo[d]thiazol-2-yl)benzyl)piperidin-4-yl)-1-oxoisoindolin-2-yl)piperidine-2,6-dione;
3-(5-(1-(isoquinolin-1-ylmethyl)piperidin-4-yl)-1-oxoisoindolin-2-yl)piperidine-2,6-dione;
3-(5-(1-(4-(4-methoxypiperidin-1-yl)benzyl)piperidin-4-yl)-1-oxoisoindolin-2-yl)piperidine-2,6-dione;
3-(5-(1-(4-(isopropylthio)benzyl)piperidin-4-yl)-1-oxoisoindolin-2-yl)piperidine-2,6-dione;
3-(1-oxo-5-(1-((S)-1-phenylethyl)piperidin-4-yl)isoindolin-2-yl)piperidine-2,6-dione;
2-(4-((4-(2-(2,6-dioxopiperidin-3-yl)-1-oxoisoindolin-5-yl)piperidin-1-yl)methyl)phenyl)acetic acid;
3-(5-(1-((7-fluoroquinolin-2-yl)methyl)piperidin-4-yl)-1-oxoisoindolin-2-yl)piperidine-2,6-dione;
3-(5-(1-((2-amino-4-(trifluoromethyl)thiazol-5-yl)methyl)piperidin-4-yl)-1-oxoisoindolin-2-yl)piperidine-2,6-dione;
3-((4-(2-(2,6-dioxopiperidin-3-yl)-1-oxoisoindolin-5-yl)piperidin-1-yl)methyl)-1,2,4-oxadiazole-5-carboxamide;
3-(5-(1-(3-(morpholinosulfonyl)benzyl)piperidin-4-yl)-1-oxoisoindolin-2-yl)piperidine-2,6-dione;
4-((4-(2-(2,6-dioxopiperidin-3-yl)-1-oxoisoindolin-5-yl)piperidin-1-yl)methyl)-N,N-dimethylbenzenesulfonamide;
3-(1-oxo-5-(1-(thiazol-4-ylmethyl)piperidin-4-yl)isoindolin-2-yl)piperidine-2,6-dione;
3-(1-oxo-5-(1-(quinoxalin-6-ylmethyl)piperidin-4-yl)isoindolin-2-yl)piperidine-2,6-dione;
3-(5-(1-(4-(tert-butyl)benzoyl)piperidin-4-yl)-1-oxoisoindolin-2-yl)piperidine-2,6-dione;
3-(5-(1-(4-((4-fluorobenzyl)oxy)benzyl)piperidin-4-yl)-1-oxoisoindolin-2-yl)piperidine-2,6-dione;
3-(5-(1-((3-methylisoxazol-5-yl)methyl)piperidin-4-yl)-1-oxoisoindolin-2-yl)piperidine-2,6-dione;
3-(5-(1-(isoxazol-3-ylmethyl)piperidin-4-yl)-1-oxoisoindolin-2-yl)piperidine-2,6-dione;
3-(1-oxo-5-(1-((R)-1-phenylethyl)piperidin-4-yl)isoindolin-2-yl)piperidine-2,6-dione;
3-(5-(1-(4-(methoxymethyl)benzyl)piperidin-4-yl)-1-oxoisoindolin-2-yl)piperidine-2,6-dione;
3-(5-(1-((S)-2-hydroxy-1-phenylethyl)piperidin-4-yl)-1-oxoisoindolin-2-yl)piperidine-2,6-dione;
3-(1-oxo-5-(1-(phenylsulfonyl)piperidin-4-yl)isoindolin-2-yl)piperidine-2,6-dione;
3-(5-(1-((5-methyl-3-phenylisoxazol-4-yl)methyl)piperidin-4-yl)-1-oxoisoindolin-2-yl)piperidine-2,6-dione;
3-(5-(1-(4-((difluoromethyl)sulfonyl)benzyl)piperidin-4-yl)-1-oxoisoindolin-2-yl)piperidine-2,6-dione;
3-(1-oxo-5-(1-(2,2,2-trifluoroethyl)piperidin-4-yl)isoindolin-2-yl)piperidine-2,6-dione;
methyl 2-((4-(2-(2,6-dioxopiperidin-3-yl)-1-oxoisoindolin-5-yl)piperidin-1-yl)methyl)oxazole-4-carboxylate;
3-(1-oxo-5-(1-(4-(pyridin-2-ylmethoxy)benzyl)piperidin-4-yl)isoindolin-2-yl)piperidine-2,6-dione;
3-(5-(1-acetylpiperidin-4-yl)-1-oxoisoindolin-2-yl)piperidine-2,6-dione;
3-(1-oxo-5-(1-((2-oxo-2,3-dihydro-1H-benzo[d]imidazol-5-yl)methyl)piperidin-4-yl)isoindolin-2-yl)piperidine-2,6-dione;
3-(5-(1-benzylpyrrolidin-3-yl)-1-oxoisoindolin-2-yl)piperidine-2,6-dione;
(R)-3-(5-((R)-1-benzylazepan-4-yl)-1-oxoisoindolin-2-yl)piperidine-2,6-dione;
(S)-3-(5-((S)-1-benzylazepan-4-yl)-1-oxoisoindolin-2-yl)piperidine-2,6-dione;
3-(5-(1-benzylazepan-4-yl)-1-oxoisoindolin-2-yl)piperidine-2,6-dione;
3-(5-(1-methyl-2,3,6,7-tetrahydro-1H-azepin-4-yl)-1-oxoisoindolin-2-yl)piperidine-2,6-dione;
3-(5-(8-benzyl-8-azabicyclo[3.2.1]octan-3-yl)-1-oxoisoindolin-2-yl)piperidine-2,6-dione;
3-(5-(8-azabicyclo[3.2.1]octan-3-yl)-1-oxoisoindolin-2-yl)piperidine-2,6-dione;
3-(5-(1-acetyl-1,2,5,6-tetrahydropyridin-3-yl)-1-oxoisoindolin-2-yl)piperidine-2,6-dione; (R)-3-(5-((R)-1-acetylpyrrolidin-3-yl)-1-oxoisoindolin-2-yl)piperidine-2,6-dione;
3-(5-(1-acetyl-1,2,3,6-tetrahydropyridin-4-yl)-1-oxoisoindolin-2-yl)piperidine-2,6-dione;
3-(5-(octahydroindolizin-7-yl)-1-oxoisoindolin-2-yl)piperidine-2,6-dione;
(R)-3-(5-((S)-1-benzylazepan-4-yl)-1-oxoisoindolin-2-yl)piperidine-2,6-dione;
3-(5-((R)-1-benzylazepan-4-yl)-1-oxoisoindolin-2-yl)piperidine-2,6-dione;
3-(5-(1-acetyl-2,5-dihydro-1H-pyrrol-3-yl)-1-oxoisoindolin-2-yl)piperidine-2,6-dione;
3-(5-(1-methylazepan-4-yl)-1-oxoisoindolin-2-yl)piperidine-2,6-dione;
(S)-3-(5-((R)-1-benzylazepan-4-yl)-1-oxoisoindolin-2-yl)piperidine-2,6-dione;
(S)-3-(5-((R)-1-acetylpyrrolidin-3-yl)-1-oxoisoindolin-2-yl)piperidine-2,6-dione;
3-(5-(1-((6-isopropoxypyridin-3-yl)methyl)piperidin-4-yl)-1-oxoisoindolin-2-yl)piperidine-2,6-dione;
3-(1-oxo-5-(1-((1-phenyl-1H-pyrazol-5-yl)methyl)piperidin-4-yl)isoindolin-2-yl)piperidine-2,6-dione;
3-(5-(1-(4-ethoxybenzyl)piperidin-4-yl)-1-oxoisoindolin-2-yl)piperidine-2,6-dione;
3-(1-oxo-5-(1-((1-phenyl-1H-pyrazol-4-yl)methyl)piperidin-4-yl)isoindolin-2-yl)piperidine-2,6-dione;
3-(5-(1-((1-isopropyl-1H-pyrazol-5-yl)methyl)piperidin-4-yl)-1-oxoisoindolin-2-yl)piperidine-2,6-dione;
3-(5-(1-(isothiazol-5-ylmethyl)piperidin-4-yl)-1-oxoisoindolin-2-yl)piperidine-2,6-dione;
3-(5-(1-((1-isopropyl-1H-pyrazol-4-yl)methyl)piperidin-4-yl)-1-oxoisoindolin-2-yl)piperidine-2,6-dione;
3-(5-(1-((1H-pyrazol-5-yl)methyl)piperidin-4-yl)-1-oxoisoindolin-2-yl)piperidine-2,6-dione;
3-(5-(1-((5-isopropoxypyridin-2-yl)methyl)piperidin-4-yl)-1-oxoisoindolin-2-yl)piperidine-2,6-dione;
3-(1-oxo-5-(1-((1-(pyridin-3-yl)-1H-pyrazol-5-yl)methyl)piperidin-4-yl)isoindolin-2-yl)piperidine-2,6-dione;
3-(1-oxo-5-(1-((1-(pyridin-3-yl)-1H-pyrazol-4-yl)methyl)piperidin-4-yl)isoindolin-2-yl)piperidine-2,6-dione;
5-((4-(2-(2,6-dioxopiperidin-3-yl)-1-oxoisoindolin-5-yl)piperidin-1-yl)methyl)-2-fluorobenzonitrile;
3-(5-(1-((5-fluoropyridin-2-yl)methyl)piperidin-4-yl)-1-oxoisoindolin-2-yl)piperidine-2,6-dione;
3-(5-(1-((1-ethyl-3-(pyridin-3-yl)-1H-pyrazol-4-yl)methyl)piperidin-4-yl)-1-oxoisoindolin-2-yl)piperidine-2,6-dione;
trans-3-(1-oxo-5-(1-((4-(trifluoromethyl)cyclohexyl)methyl)piperidin-4-yl)isoindolin-2-yl)piperidine-2,6-dione;
cis-3-(1-oxo-5-(1-((4-(trifluoromethyl)cyclohexyl)methyl)piperidin-4-yl)isoindolin-2-yl)piperidine-2,6-dione;
trans-3-(5-(1-((4-methoxycyclohexyl)methyl)piperidin-4-yl)-1-oxoisoindolin-2-yl)piperidine-2,6-dione; and
3-(5-(1-((6-methoxypyridin-2-yl)methyl)piperidin-4-yl)-1-oxoisoindolin-2-yl)piperidine-2,6-dione;
   or a pharmaceutically acceptable salt, hydrate, solvate, prodrug, stereoisomer, or tautomer thereof.

Other IKZF2 inhibitors are described in U.S. Pat. No. 10,414,755, which is incorporated by reference in its entirety.

In some embodiments, the IKZF2 inhibitor comprises a compound of Formula (Ic):

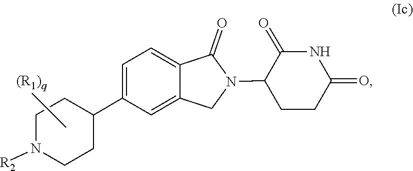

or a pharmaceutically acceptable salt, hydrate, solvate, prodrug, stereoisomer, or tautomer thereof,
wherein:
each R₁ is independently (C₁-C₆)alkyl;
R₂ is (C₁-C₆)alkyl substituted with one to three R₄;
each R₄ is independently selected from (C₆-C₁₀)aryl and (C₃-C₈)cycloalkyl, wherein the aryl and cycloalkyl groups are optionally substituted with one or more R₇;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, CN, (C₆-C₁₀)aryl, 5- or 6-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN; and
q is 0.

In some embodiments, R₄ is phenyl or (C₃-C₈)cycloalkyl optionally substituted with one to three R₇. In some embodiments, R₄ is phenyl optionally substituted with one to three R₇. In some embodiments, R₄ is (C₃-C₈)cycloalkyl optionally substituted with one to three R₇.

In some embodiments, the IKZF2 inhibitor comprises a compound selected from:

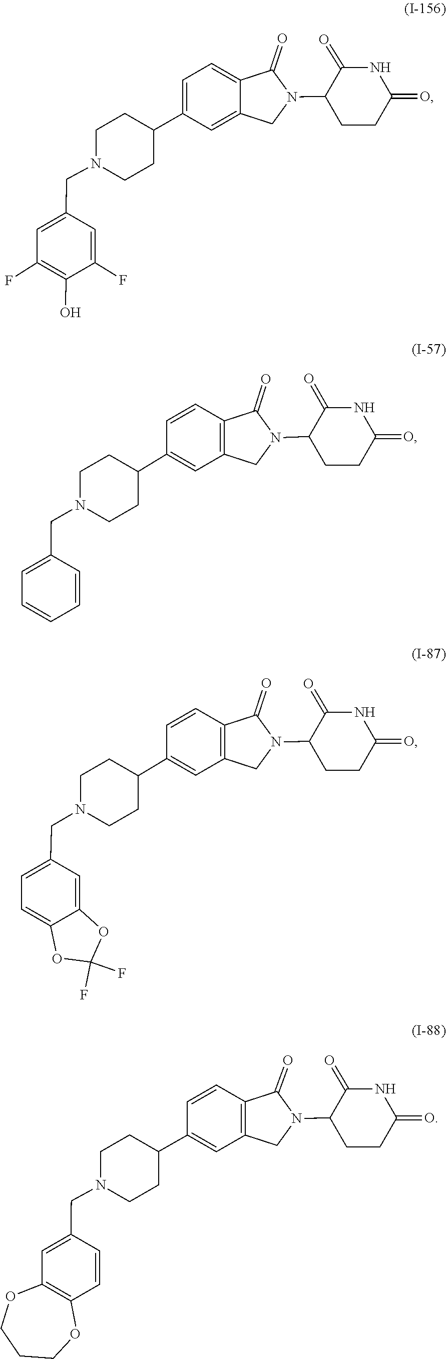 [SMILES: O=C1CCC(N2Cc3cc(C4CCN(Cc5cc(F)c(O)c(F)c5)CC4)ccc3C2=O)C(=O)N1 ; O=C1CCC(N2Cc3cc(C4CCN(Cc5ccccc5)CC4)ccc3C2=O)C(=O)N1 ; O=C1CCC(N2Cc3cc(C4CCN(Cc5ccc6c(c5)OC(F)(F)O6)CC4)ccc3C2=O)C(=O)N1 ; O=C1CCC(N2Cc3cc(C4CCN(Cc5ccc6c(c5)OCCCO6)CC4)ccc3C2=O)C(=O)N1]

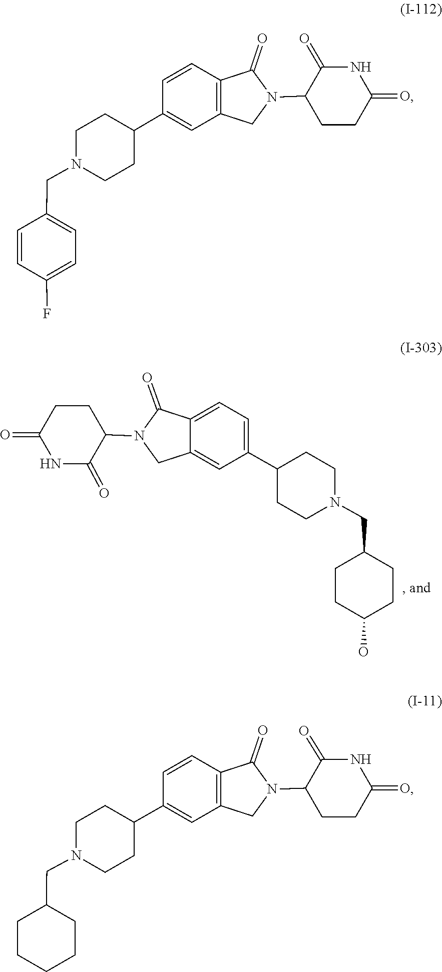 [SMILES: O=C1CCC(N2Cc3cc(C4CCN(Cc5ccc(F)cc5)CC4)ccc3C2=O)C(=O)N1 ; O=C1CCC(N2Cc3cc(C4CCN(C[C@H]5CC[C@H](O)CC5)CC4)ccc3C2=O)C(=O)N1 ; O=C1CCC(N2Cc3cc(C4CCN(CC5CCCCC5)CC4)ccc3C2=O)C(=O)N1]

or a pharmaceutically acceptable salt, hydrate, solvate, prodrug, stereoisomer, or tautomer thereof.

In some embodiments, the compound is compound (I-156):

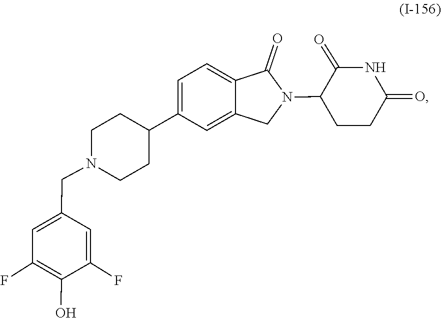 [SMILES: O=C1CCC(N2Cc3cc(C4CCN(Cc5cc(F)c(O)c(F)c5)CC4)ccc3C2=O)C(=O)N1]

or a pharmaceutically acceptable salt, hydrate, solvate, prodrug, stereoisomer, or tautomer thereof.

In some embodiments, the compound is compound (I-57):

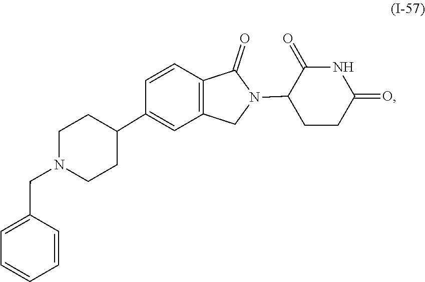 [SMILES: O=C1CCC(N2Cc3cc(C4CCN(Cc5ccccc5)CC4)ccc3C2=O)C(=O)N1]

or a pharmaceutically acceptable salt, hydrate, solvate, prodrug, stereoisomer, or tautomer thereof.

In some embodiments, the compound is compound (I-87):

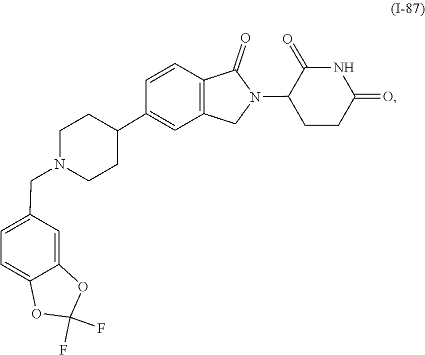 [SMILES: O=C1CCC(N2Cc3cc(C4CCN(Cc5ccc6c(c5)OC(F)(F)O6)CC4)ccc3C2=O)C(=O)N1]

or a pharmaceutically acceptable salt, hydrate, solvate, prodrug, stereoisomer, or tautomer thereof.

In some embodiments, the compound is compound (I-88):

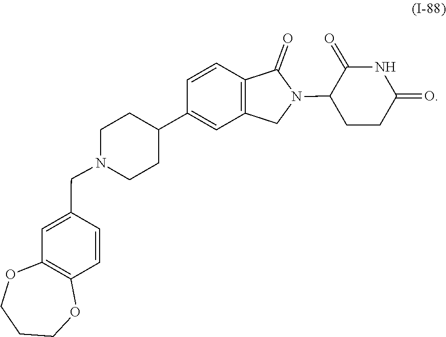 [SMILES: O=C1CCC(N2Cc3cc(C4CCN(Cc5ccc6c(c5)OCCCO6)CC4)ccc3C2=O)C(=O)N1]

or a pharmaceutically acceptable salt, hydrate, solvate, prodrug, stereoisomer, or tautomer thereof.

In some embodiments, the compound is compound (I-112):

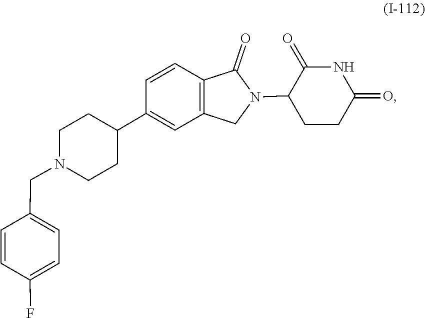 [SMILES: O=C1CCC(N2Cc3cc(C4CCN(Cc5ccc(F)cc5)CC4)ccc3C2=O)C(=O)N1]

or a pharmaceutically acceptable salt, hydrate, solvate, prodrug, stereoisomer, or tautomer thereof.

In some embodiments, the compound is compound (I-303):

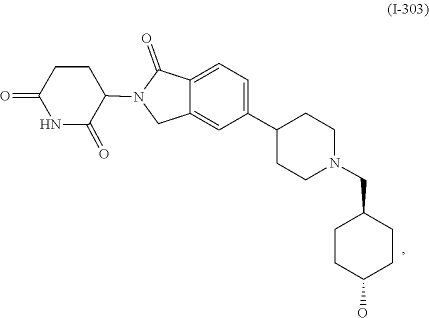 [SMILES: O=C1CCC(N2Cc3cc(C4CCN(C[C@H]5CC[C@H](O)CC5)CC4)ccc3C2=O)C(=O)N1]

or a pharmaceutically acceptable salt, hydrate, solvate, prodrug, stereoisomer, or tautomer thereof.

In some embodiments, the compound is compound (I-11):

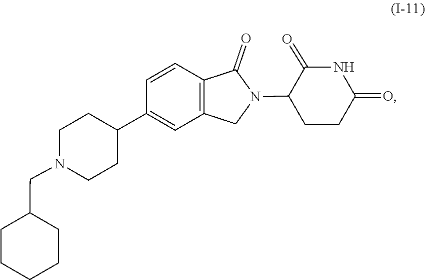 [SMILES: O=C1CCC(N2Cc3cc(C4CCN(CC5CCCCC5)CC4)ccc3C2=O)C(=O)N1]

or a pharmaceutically acceptable salt, hydrate, solvate, prodrug, stereoisomer, or tautomer thereof.

In some embodiments, the IKZF2 inhibitor comprises a compound of Formula (Ic):

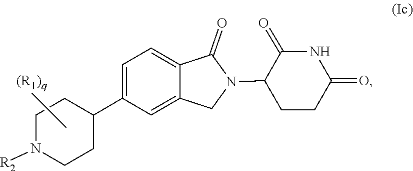

or a pharmaceutically acceptable salt, hydrate, solvate, prodrug, stereoisomer, or tautomer thereof,
wherein:
each R₁ is independently (C₁-C₆)alkyl;
R₂ is (C₁-C₆)alkyl substituted with one to three R₄;
each R₄ is independently selected from phenyl or 5- or 6-membered heteroaryl comprising 1 to 4 heteroatoms selected from O, N, and S, wherein the phenyl and heteroaryl groups are optionally substituted with one to three R₇;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, CN, (C₆-C₁₀)aryl, 5- or 6-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN; and
q is 0.

In some embodiments, R₄ is phenyl substituted with one to three R₇. In some embodiments, R₄ is 5-membered heteroaryl comprising 1 to 3 heteroatoms selected from O, N, and S, are optionally substituted with one to three R₇. In some embodiments, R₄ is 6-membered heteroaryl comprising 1 to 3 heteroatoms selected from O, N, and S, optionally substituted with one to three R₇.

In some embodiments, the compound is selected from:

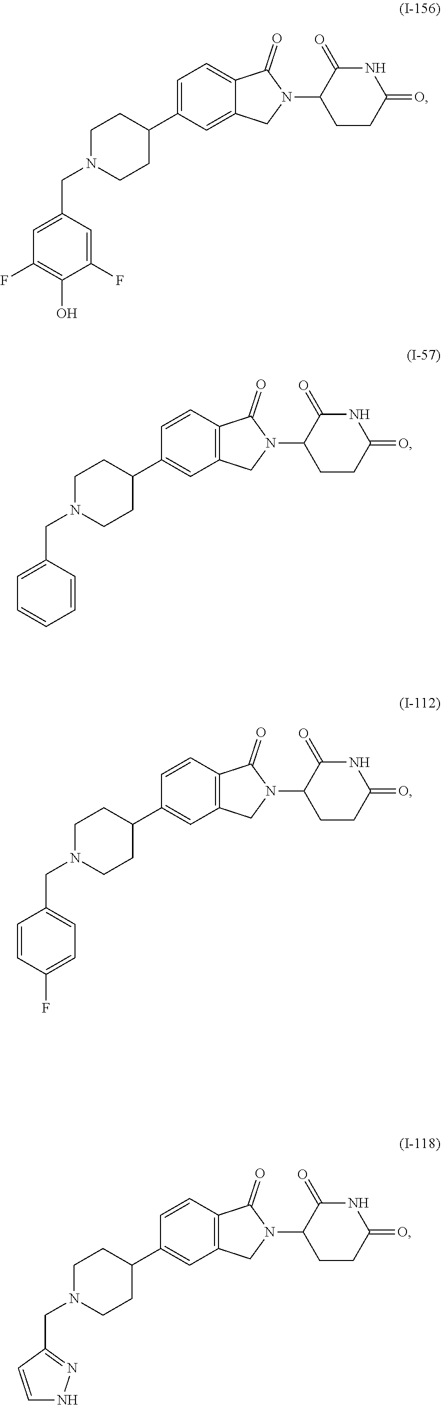 [SMILES: O=C1CCC(N2Cc3cc(C4CCN(Cc5cc(F)c(O)c(F)c5)CC4)ccc3C2=O)C(=O)N1 ; O=C1CCC(N2Cc3cc(C4CCN(Cc5ccccc5)CC4)ccc3C2=O)C(=O)N1 ; O=C1CCC(N2Cc3cc(C4CCN(Cc5ccc(F)cc5)CC4)ccc3C2=O)C(=O)N1 ; O=C1CCC(N2Cc3cc(C4CCN(Cc5cc[nH]n5)CC4)ccc3C2=O)C(=O)N1]

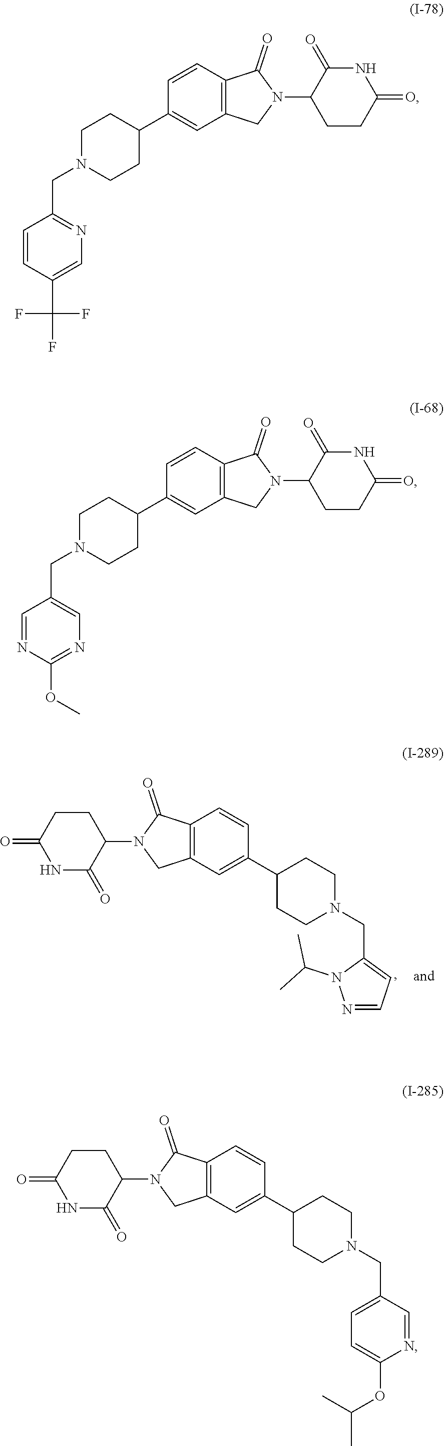 [SMILES: O=C1CCC(N2Cc3cc(C4CCN(Cc5ccc(C(F)(F)F)cn5)CC4)ccc3C2=O)C(=O)N1 ; COc1ncc(CN2CCC(c3ccc4c(c3)CN(C3CCC(=O)NC3=O)C4=O)CC2)cn1 ; CC(C)n1nccc1CN1CCC(c2ccc3c(c2)CN(C2CCC(=O)NC2=O)C3=O)CC1 ; CC(C)Oc1ccc(CN2CCC(c3ccc4c(c3)CN(C3CCC(=O)NC3=O)C4=O)CC2)cn1]

or a pharmaceutically acceptable salt, hydrate, solvate, prodrug, stereoisomer, or tautomer thereof.

In some embodiments, the IKZF2 inhibitor comprises a compound of Formula (I′):

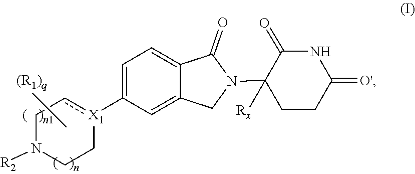

or a pharmaceutically acceptable salt, hydrate, solvate, prodrug, stereoisomer, or tautomer thereof,
wherein:
X₁ is CR₃;
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen, or
two R₁ together with the carbon atoms to which they are attached form a 5- or 6-membered heterocycloalkyl ring, or
two R₁, when on adjacent atoms, together with the atoms to which they are attached form a (C₆-C₁₀)aryl ring or a 5- or 6-membered heteroaryl ring comprising 1 to 3 heteroatoms selected from O, N, and S;
R₂ is (C₁-C₆)alkyl, —C(O)(C₁-C₆)alkyl, —C(O)(CH₂)₀₋₃(C₆-C₁₀)aryl, —C(O)O(CH₂)₀₋₃(C₆-C₁₀)aryl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₁ and R₂, when on adjacent atoms, together with the atoms to which they are attached form a 5- or 6-membered heterocycloalkyl ring;
R₃ is H or R₃ is absent when [symbol] is a double bond;
each R₄ is independently selected from —C(O)OR₆, —C(O)NR₆R₆, —NR₆C(O)R₆, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₈ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆ are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more Rₗₗ, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a=(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a=(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D;
p is 0, 1, or 2;
n is 0, 1, or 2;
n1 is 1 or 2, wherein n+n1≤3; and
q is 0, 1, 2, 3, or 4.

In some embodiments, the compound has a Formula (I):

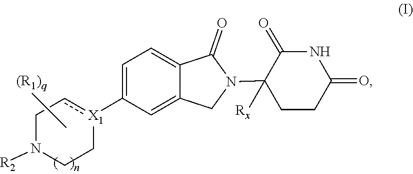

or a pharmaceutically acceptable salt, hydrate, solvate, prodrug, stereoisomer, or tautomer thereof,
wherein:
X₁ is CR₃; [symbol]
[symbol] is optionally a double bond when X₁ is CR₃ and R₃ is absent;
each R₁ is independently (C₁-C₆)alkyl, (C₁-C₆)haloalkyl, (C₁-C₆)hydroxyalkyl, or halogen;
R₂ is (C₁-C₆)alkyl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅; [symbol]
R₃ is H or R₃ is absent when [symbol] is a double bond;
each R₄ is independently selected from —C(O)OR₆, —C(O)NR₆R₆, —NR₆C(O)R₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S optionally substituted with one or more R₁₀;
R₆ and R₆ are each independently H or (C₁-C₆)alkyl;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, CN, (C₆-C₁₀)aryl, 5- or 6-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
Rₓ is H or D;
n is 1 or 2; and
q is 0, 1, 2, 3, or 4.

In some embodiments, n is 1. In some embodiments, n is 2. In some embodiments, q is 0, 1, or 2. In some embodiments, X₁ is CH. In some embodiments, RX is H.

In some embodiments, R₂ is (C₆-C₁₀)aryl, (C₃-C₈)cycloalkyl, or 5- to 7-membered heterocycloalkyl comprising 1 to 3 heteroatoms selected from O, N, and S, wherein the aryl, heteroaryl, cycloalkyl, and heterocycloalkyl are optionally substituted with one to three R₅. In some embodiments, R₂ is (C₆-C₁₀)aryl, (C₃-C₈)cycloalkyl, or 5- to 7-membered heterocycloalkyl comprising 1 to 3 heteroatoms selected from O, N, and S. In some embodiments, R₂ is (C₁-C₆)alkyl optionally substituted with one to three R₄.

In some embodiments, the compound has a Formula (Ia), Formula (Ib), Formula (Ic), or Formula (Id):

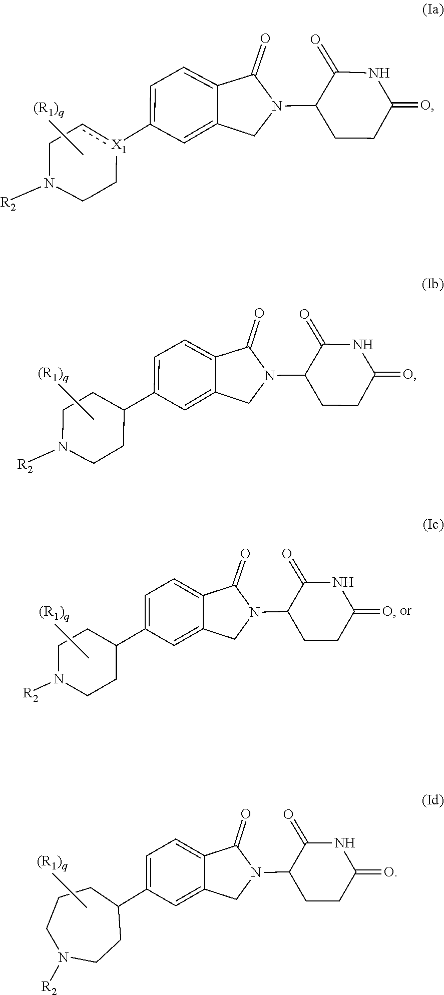

In some embodiments, R₂ is (C₆-C₁₀)aryl, (C₃-C₈)cycloalkyl, or 5- to 7-membered heterocycloalkyl comprising 1 to 3 heteroatoms selected from O, N, and S, wherein the aryl, heteroaryl, cycloalkyl, and heterocycloalkyl are optionally substituted with one to three R₅. In some embodiments, R₂ is (C₆-C₁₀)aryl, (C₃-C₈)cycloalkyl, or 5- to 7-membered heterocycloalkyl comprising 1 to 3 heteroatoms selected from O, N, and S. In some embodiments, R₂ is (C₁-C₆)alkyl optionally substituted with one to three R₄.

In some embodiments, the compound is selected from:
3-(5-(1-ethylpiperidin-4-yl)-1-oxoisoindolin-2-yl)piperidine-2,6-dione;
3-(1-oxo-5-(1-propylpiperidin-4-yl)isoindolin-2-yl)piperidine-2,6-dione;
3-(5-(1-(cyclopropylmethyl)piperidin-4-yl)-1-oxoisoindolin-2-yl)piperidine-2,6-dione;
3-(5-(1-isobutylpiperidin-4-yl)-1-oxoisoindolin-2-yl)piperidine-2,6-dione;
3-(5-(1-(cyclobutylmethyl)piperidin-4-yl)-1-oxoisoindolin-2-yl)piperidine-2,6-dione;
3-(5-(1-(oxazol-2-ylmethyl)piperidin-4-yl)-1-oxoisoindolin-2-yl)piperidine-2,6-dione;
3-(1-oxo-5-(1-(thiazol-2-ylmethyl)piperidin-4-yl)isoindolin-2-yl)piperidine-2,6-dione;
3-(5-(1-(cyclopentylmethyl)piperidin-4-yl)-1-oxoisoindolin-2-yl)piperidine-2,6-dione;
3-(5-(1-((5-chlorothiophen-2-yl)methyl)piperidin-4-yl)-1-oxoisoindolin-2-yl)piperidine-2,6-dione;
3-(5-(1-((2-chlorothiazol-5-yl)methyl)piperidin-4-yl)-1-oxoisoindolin-2-yl)piperidine-2,6-dione;
3-(5-(1-(cyclohexylmethyl)piperidin-4-yl)-1-oxoisoindolin-2-yl)piperidine-2,6-dione;
3-(1-oxo-5-(1-(2-(pyrrolidin-1-yl)ethyl)piperidin-4-yl)isoindolin-2-yl)piperidine-2,6-dione;
3-(1-oxo-5-(1-((tetrahydro-2H-pyran-4-yl)methyl)piperidin-4-yl)isoindolin-2-yl)piperidine-2,6-dione;
3-(1-oxo-5-(1-phenethylpiperidin-4-yl)isoindolin-2-yl)piperidine-2,6-dione;
3-(5-(1-(3-fluorobenzyl)piperidin-4-yl)-1-oxoisoindolin-2-yl)piperidine-2,6-dione;
3-(5-(1-(3-chlorobenzyl)piperidin-4-yl)-1-oxoisoindolin-2-yl)piperidine-2,6-dione;
3-(5-(1-(2-fluorobenzyl)piperidin-4-yl)-1-oxoisoindolin-2-yl)piperidine-2,6-dione;
3-(5-(1-(2-chlorobenzyl)piperidin-4-yl)-1-oxoisoindolin-2-yl)piperidine-2,6-dione;
3-(1-oxo-5-(1-(2-(piperidin-1-yl)ethyl)piperidin-4-yl)isoindolin-2-yl)piperidine-2,6-dione;
3-(5-(1-((3,5-dimethylisoxazol-4-yl)methyl)piperidin-4-yl)-1-oxoisoindolin-2-yl)piperidine-2,6-dione;
3-(5-(1-((1,3-dimethyl-1H-pyrazol-5-yl)methyl)piperidin-4-yl)-1-oxoisoindolin-2-yl)piperidine-2,6-dione;
3-(5-(1-((6-methylpyridin-2-yl)methyl)piperidin-4-yl)-1-oxoisoindolin-2-yl)piperidine-2,6-dione;
3-(5-(1-(3-morpholinopropyl)piperidin-4-yl)-1-oxoisoindolin-2-yl)piperidine-2,6-dione;
3-(5-(1-(2,6-difluorobenzyl)piperidin-4-yl)-1-oxoisoindolin-2-yl)piperidine-2,6-dione;
3-(5-(1-(2,6-dichlorobenzyl)piperidin-4-yl)-1-oxoisoindolin-2-yl)piperidine-2,6-dione;
3-(5-(1-(3,5-difluorobenzyl)piperidin-4-yl)-1-oxoisoindolin-2-yl)piperidine-2,6-dione;
3-(5-(1-(3,5-dibromobenzyl)piperidin-4-yl)-1-oxoisoindolin-2-yl)piperidine-2,6-dione;
3-(5-(1-(3-chloro-5-fluorobenzyl)piperidin-4-yl)-1-oxoisoindolin-2-yl)piperidine-2,6-dione;
3-(5-(1-(2,5-difluorobenzyl)piperidin-4-yl)-1-oxoisoindolin-2-yl)piperidine-2,6-dione;
3-(5-(1-(2,5-dichlorobenzyl)piperidin-4-yl)-1-oxoisoindolin-2-yl)piperidine-2,6-dione;
4-((4-(2-(2,6-dioxopiperidin-3-yl)-1-oxoisoindolin-5-yl)piperidin-1-yl)methyl)benzonitrile;
3-(5-(1-(4-(hydroxymethyl)benzyl)piperidin-4-yl)-1-oxoisoindolin-2-yl)piperidine-2,6-dione;
3-(5-(1-(3,4-dichlorobenzyl)piperidin-4-yl)-1-oxoisoindolin-2-yl)piperidine-2,6-dione;
3-(5-(1-(4-chloro-2-fluorobenzyl)piperidin-4-yl)-1-oxoisoindolin-2-yl)piperidine-2,6-dione;
3-(5-(1-(2-chloro-4-fluorobenzyl)piperidin-4-yl)-1-oxoisoindolin-2-yl)piperidine-2,6-dione;
3-((4-(2-(2,6-dioxopiperidin-3-yl)-1-oxoisoindolin-5-yl)piperidin-1-yl)methyl)benzonitrile;
3-(5-(1-(2,3-difluorobenzyl)piperidin-4-yl)-1-oxoisoindolin-2-yl)piperidine-2,6-dione;
2-((4-(2-(2,6-dioxopiperidin-3-yl)-1-oxoisoindolin-5-yl)piperidin-1-yl)methyl)benzonitrile;
3-(5-(1-(4-methoxybenzyl)piperidin-4-yl)-1-oxoisoindolin-2-yl)piperidine-2,6-dione;
3-(5-(1-(2,5-dimethylbenzyl)piperidin-4-yl)-1-oxoisoindolin-2-yl)piperidine-2,6-dione;
3-(5-(1-(3,4-dimethylbenzyl)piperidin-4-yl)-1-oxoisoindolin-2-yl)piperidine-2,6-dione;
3-(5-(1-(2,4-dimethylbenzyl)piperidin-4-yl)-1-oxoisoindolin-2-yl)piperidine-2,6-dione;
3-(5-(1-((1H-indazol-4-yl)methyl)piperidin-4-yl)-1-oxoisoindolin-2-yl)piperidine-2,6-dione;
3-(5-(1-((1H-benzo[d]imidazol-2-yl)methyl)piperidin-4-yl)-1-oxoisoindolin-2-yl)piperidine-2,6-dione;
3-(5-(1-(4-isopropylbenzyl)piperidin-4-yl)-1-oxoisoindolin-2-yl)piperidine-2,6-dione;
methyl 5-((4-(2-(2,6-dioxopiperidin-3-yl)-1-oxoisoindolin-5-yl)piperidin-1-yl)methyl)furan-2-carboxylate;
3-(5-(1-(naphthalen-2-ylmethyl)piperidin-4-yl)-1-oxoisoindolin-2-yl)piperidine-2,6-dione;
3-(1-oxo-5-(1-(quinolin-2-ylmethyl)piperidin-4-yl)isoindolin-2-yl)piperidine-2,6-dione;
3-(5-(1-(naphthalen-1-ylmethyl)piperidin-4-yl)-1-oxoisoindolin-2-yl)piperidine-2,6-dione;
3-(5-(1-((1-methyl-1H-benzo[d]imidazol-2-yl)methyl)piperidin-4-yl)-1-oxoisoindolin-2-yl)piperidine-2,6-dione;
3-(1-oxo-5-(1-(4-(trifluoromethoxy)benzyl)piperidin-4-yl)isoindolin-2-yl)piperidine-2,6-dione;
3-(5-(1-(4-(1H-pyrrol-1-yl)benzyl)piperidin-4-yl)-1-oxoisoindolin-2-yl)piperidine-2,6-dione;
3-(5-(1-(4-(1H-1,2,4-triazol-1-yl)benzyl)piperidin-4-yl)-1-oxoisoindolin-2-yl)piperidine-2,6-dione;
3-(1-oxo-5-(1-(3-(trifluoromethoxy)benzyl)piperidin-4-yl)isoindolin-2-yl)piperidine-2,6-dione;
3-(1-oxo-5-(1-(2-(trifluoromethoxy)benzyl)piperidin-4-yl)isoindolin-2-yl)piperidine-2,6-dione;
3-(5-(1-benzylpiperidin-4-yl)-1-oxoisoindolin-2-yl)piperidine-2,6-dione;
3-(1-oxo-5-(1-(pyridin-2-ylmethyl)piperidin-4-yl)isoindolin-2-yl)piperidine-2,6-dione;
3-(1-oxo-5-(1-(pyridin-3-ylmethyl)piperidin-4-yl)isoindolin-2-yl)piperidine-2,6-dione;
3-(1-oxo-5-(1-(pyridin-4-ylmethyl)piperidin-4-yl)isoindolin-2-yl)piperidine-2,6-dione;
3-(1-oxo-5-(1-(pyrimidin-5-ylmethyl)piperidin-4-yl)isoindolin-2-yl)piperidine-2,6-dione;
3-(1-oxo-5-(1-(1-phenylethyl)piperidin-4-yl)isoindolin-2-yl)piperidine-2,6-dione;
3-(5-(1-(4-(fluoromethyl)benzyl)piperidin-4-yl)-1-oxoisoindolin-2-yl)piperidine-2,6-dione;
3-(5-(1-(3,4-difluorobenzyl)piperidin-4-yl)-1-oxoisoindolin-2-yl)piperidine-2,6-dione;
2-((4-(2-(2,6-dioxopiperidin-3-yl)-1-oxoisoindolin-5-yl)piperidin-1-yl)methyl)pyrimidine-5-carbonitrile;
3-(5-(1-(4-ethylbenzyl)piperidin-4-yl)-1-oxoisoindolin-2-yl)piperidine-2,6-dione;
3-(5-(1-(2-methoxybenzyl)piperidin-4-yl)-1-oxoisoindolin-2-yl)piperidine-2,6-dione;
3-(5-(1-((2-methoxypyrimidin-5-yl)methyl)piperidin-4-yl)-1-oxoisoindolin-2-yl)piperidine-2,6-dione;
3-(5-(1-(3-fluoro-4-methylbenzyl)piperidin-4-yl)-1-oxoisoindolin-2-yl)piperidine-2,6-dione;
3-(5-(1-(4-(difluoromethyl)benzyl)piperidin-4-yl)-1-oxoisoindolin-2-yl)piperidine-2,6-dione;
4-((4-(2-(2,6-dioxopiperidin-3-yl)-1-oxoisoindolin-5-yl)piperidin-1-yl)methyl)benzamide;
4-((4-(2-(2,6-dioxopiperidin-3-yl)-1-oxoisoindolin-5-yl)piperidin-1-yl)methyl)benzoic acid;
3-(5-(1-(3-(difluoromethyl)benzyl)piperidin-4-yl)-1-oxoisoindolin-2-yl)piperidine-2,6-dione;
3-((4-(2-(2,6-dioxopiperidin-3-yl)-1-oxoisoindolin-5-yl)piperidin-1-yl)methyl)benzoic acid;
3-(1-oxo-5-(1-(4-propylbenzyl)piperidin-4-yl)isoindolin-2-yl)piperidine-2,6-dione;
3-(1-oxo-5-(1-(4-(trifluoromethyl)benzyl)piperidin-4-yl)isoindolin-2-yl)piperidine-2,6-dione;
3-(5-(1-(4-(difluoromethoxy)benzyl)piperidin-4-yl)-1-oxoisoindolin-2-yl)piperidine-2,6-dione;
3-(1-oxo-5-(1-((5-(trifluoromethyl)pyridin-2-yl)methyl)piperidin-4-yl)isoindolin-2-yl)piperidine-2,6-dione;
3-(5-(1-(3-(difluoromethoxy)benzyl)piperidin-4-yl)-1-oxoisoindolin-2-yl)piperidine-2,6-dione;
3-(5-(1-(2-(difluoromethoxy)benzyl)piperidin-4-yl)-1-oxoisoindolin-2-yl)piperidine-2,6-dione;
3-(5-(1-(4-cyclobutylbenzyl)piperidin-4-yl)-1-oxoisoindolin-2-yl)piperidine-2,6-dione;
3-(5-(1-((2,3-dihydrobenzo[b][1,4]dioxin-5-yl)methyl)piperidin-4-yl)-1-oxoisoindolin-2-yl)piperidine-2,6-dione;
3-(5-(1-((2,3-dihydrobenzo[b][1,4]dioxin-6-yl)methyl)piperidin-4-yl)-1-oxoisoindolin-2-yl)piperidine-2,6-dione;
3-(5-(1-(4-(tert-butyl)benzyl)piperidin-4-yl)-1-oxoisoindolin-2-yl)piperidine-2,6-dione;
3-(5-(1-(4-isobutylbenzyl)piperidin-4-yl)-1-oxoisoindolin-2-yl)piperidine-2,6-dione; N-(4-((4-(2-(2,6-dioxopiperidin-3-yl)-1-oxoisoindolin-5-yl)piperidin-1-yl)methyl)phenyl)acetamide;
3-(5-(1-((2,2-difluorobenzo[d][1,3]dioxol-5-yl)methyl)piperidin-4-yl)-1-oxoisoindolin-2-yl)piperidine-2,6-dione;
3-(5-(1-((3,4-dihydro-2H-benzo[b][1,4]dioxepin-7-yl)methyl)piperidin-4-yl)-1-oxoisoindolin-2-yl)piperidine-2,6-dione;
3-(1-oxo-5-(1-(4-(tert-pentyl)benzyl)piperidin-4-yl)isoindolin-2-yl)piperidine-2,6-dione;
3-(5-(1-([1,1′-biphenyl]-4-ylmethyl)piperidin-4-yl)-1-oxoisoindolin-2-yl)piperidine-2,6-dione;
3-(5-(1-(4-(1H-pyrazol-1-yl)benzyl)piperidin-4-yl)-1-oxoisoindolin-2-yl)piperidine-2,6-dione;
3-(5-(1-(4-(1H-imidazol-1-yl)benzyl)piperidin-4-yl)-1-oxoisoindolin-2-yl)piperidine-2,6-dione;
3-(5-(1-(3-(1H-pyrazol-1-yl)benzyl)piperidin-4-yl)-1-oxoisoindolin-2-yl)piperidine-2,6-dione;
3-(5-(1-(4-cyclohexylbenzyl)piperidin-4-yl)-1-oxoisoindolin-2-yl)piperidine-2,6-dione;
3-(1-oxo-5-(1-(pyrimidin-2-ylmethyl)piperidin-4-yl)isoindolin-2-yl)piperidine-2,6-dione;
3-(5-(1-(4-bromobenzyl)piperidin-4-yl)-1-oxoisoindolin-2-yl)piperidine-2,6-dione;
3-(5-(1-(4-chlorobenzyl)piperidin-4-yl)-1-oxoisoindolin-2-yl)piperidine-2,6-dione;
3-(5-(1-(3,5-dichlorobenzyl)piperidin-4-yl)-1-oxoisoindolin-2-yl)piperidine-2,6-dione
3-(5-(1-(4-chloro-3-fluorobenzyl)piperidin-4-yl)-1-oxoisoindolin-2-yl)piperidine-2,6-dione;
3-(5-(1-(3-chloro-4-fluorobenzyl)piperidin-4-yl)-1-oxoisoindolin-2-yl)piperidine-2,6-dione;
3-(5-(1-(2,4-difluorobenzyl)piperidin-4-yl)-1-oxoisoindolin-2-yl)piperidine-2,6-dione;
3-(5-(1-(3-methoxybenzyl)piperidin-4-yl)-1-oxoisoindolin-2-yl)piperidine-2,6-dione;
3-(5-(1-(benzo[c][1,2,5]oxadiazol-5-ylmethyl)piperidin-4-yl)-1-oxoisoindolin-2-yl)piperidine-2,6-dione;
3-(5-(1-(2-cyclopropylbenzyl)piperidin-4-yl)-1-oxoisoindolin-2-yl)piperidine-2,6-dione;
3-(5-(1-((1,3-dihydroisobenzofuran-5-yl)methyl)piperidin-4-yl)-1-oxoisoindolin-2-yl)piperidine-2,6-dione;
3-(1-oxo-5-(1-(2-(trifluoromethyl)benzyl)piperidin-4-yl)isoindolin-2-yl)piperidine-2,6-dione;
3-(5-(1-(3-isopropoxybenzyl)piperidin-4-yl)-1-oxoisoindolin-2-yl)piperidine-2,6-dione;
3-(1-oxo-5-(1-(4-(thiophen-3-yl)benzyl)piperidin-4-yl)isoindolin-2-yl)piperidine-2,6-dione;
3-(5-(1-(4-cyclopentylbenzyl)piperidin-4-yl)-1-oxoisoindolin-2-yl)piperidine-2,6-dione;
3-(1-oxo-5-(1-(4-(pyrrolidin-1-yl)benzyl)piperidin-4-yl)isoindolin-2-yl)piperidine-2,6-dione;
3-(5-(1-(4-fluorobenzyl)piperidin-4-yl)-1-oxoisoindolin-2-yl)piperidine-2,6-dione;
3-(5-(1-(2,4-dichlorobenzyl)piperidin-4-yl)-1-oxoisoindolin-2-yl)piperidine-2,6-dione;
3-(1-oxo-5-(1-(quinolin-8-ylmethyl)piperidin-4-yl)isoindolin-2-yl)piperidine-2,6-dione;
3-(5-(1-((1-methyl-1H-pyrazol-4-yl)methyl)piperidin-4-yl)-1-oxoisoindolin-2-yl)piperidine-2,6-dione;
3-(5-(1-((1H-pyrazol-4-yl)methyl)piperidin-4-yl)-1-oxoisoindolin-2-yl)piperidine-2,6-dione;
3-(5-(1-((1-methyl-1H-pyrazol-3-yl)methyl)piperidin-4-yl)-1-oxoisoindolin-2-yl)piperidine-2,6-dione;
3-(5-(1-((1H-pyrazol-3-yl)methyl)piperidin-4-yl)-1-oxoisoindolin-2-yl)piperidine-2,6-dione;
3-(5-(1-((1H-pyrrol-3-yl)methyl)piperidin-4-yl)-1-oxoisoindolin-2-yl)piperidine-2,6-dione;
3-(5-(1-((1H-imidazol-5-yl)methyl)piperidin-4-yl)-1-oxoisoindolin-2-yl)piperidine-2,6-dione;
3-(5-(1-((1-ethyl-1H-pyrazol-3-yl)methyl)piperidin-4-yl)-1-oxoisoindolin-2-yl)piperidine-2,6-dione;
3-(5-(1-((2-aminopyrimidin-5-yl)methyl)piperidin-4-yl)-1-oxoisoindolin-2-yl)piperidine-2,6-dione;
3-(5-(1-((6-aminopyridin-3-yl)methyl)piperidin-4-yl)-1-oxoisoindolin-2-yl)piperidine-2,6-dione;
3-(5-(1-((5-amino-1-methyl-1H-pyrazol-4-yl)methyl)piperidin-4-yl)-1-oxoisoindolin-2-yl)piperidine-2,6-dione;
3-(5-(1-((6-methylimidazo[2,1-b]thiazol-5-yl)methyl)piperidin-4-yl)-1-oxoisoindolin-2-yl)piperidine-2,6-dione;
3-(5-(1-(imidazo[1,2-a]pyrazin-3-ylmethyl)piperidin-4-yl)-1-oxoisoindolin-2-yl)piperidine-2,6-dione;
3-(5-(1-([1,2,4]triazolo[1,5-a]pyridin-5-ylmethyl)piperidin-4-yl)-1-oxoisoindolin-2-yl)piperidine-2,6-dione;
3-(1-oxo-5-(1-(pyrazolo[1,5-a]pyridin-4-ylmethyl)piperidin-4-yl)isoindolin-2-yl)piperidine-2,6-dione;
3-(5-(1-((1,4-dimethyl-1H-imidazol-2-yl)methyl)piperidin-4-yl)-1-oxoisoindolin-2-yl)piperidine-2,6-dione;
3-(5-(1-(benzo[d]thiazol-5-ylmethyl)piperidin-4-yl)-1-oxoisoindolin-2-yl)piperidine-2,6-dione;
3-(1-oxo-5-(1-(pyrazolo[1,5-a]pyrimidin-6-ylmethyl)piperidin-4-yl)isoindolin-2-yl)piperidine-2,6-dione;
3-(5-(1-(imidazo[1,2-a]pyrimidin-3-ylmethyl)piperidin-4-yl)-1-oxoisoindolin-2-yl)piperidine-2,6-dione;
3-(5-(1-(imidazo[1,2-a]pyrimidin-2-ylmethyl)piperidin-4-yl)-1-oxoisoindolin-2-yl)piperidine-2,6-dione;
3-(5-(1-((1-cyclobutyl-1H-1,2,3-triazol-4-yl)methyl)piperidin-4-yl)-1-oxoisoindolin-2-yl)piperidine-2,6-dione;
3-(1-oxo-5-(1-((4,5,6,7-tetrahydropyrazolo[1,5-a]pyridin-2-yl)methyl)piperidin-4-yl)isoindolin-2-yl)piperidine-2,6-dione;
3-(5-(1-((1H-indol-2-yl)methyl)piperidin-4-yl)-1-oxoisoindolin-2-yl)piperidine-2,6-dione;
3-(5-(1-((1H-indazol-6-yl)methyl)piperidin-4-yl)-1-oxoisoindolin-2-yl)piperidine-2,6-dione;
3-(5-(1-((1H-pyrrolo[2,3-b]pyridin-3-yl)methyl)piperidin-4-yl)-1-oxoisoindolin-2-yl)piperidine-2,6-dione; 3-((4-(2-(2,6-dioxopiperidin-3-yl)-1-oxoisoindolin-5-yl)piperidin-1-yl)methyl)benzamide;
3-(5-(1-((1H-pyrrolo[2,3-b]pyridin-6-yl)methyl)piperidin-4-yl)-1-oxoisoindolin-2-yl)piperidine-2,6-dione;
3-(5-(1-((3,4-dihydro-2H-benzo[b][1,4]thiazin-6-yl)methyl)piperidin-4-yl)-1-oxoisoindolin-2-yl)piperidine-2,6-dione;
3-(1-oxo-5-(1-((2-(pyrrolidin-1-yl)pyrimidin-5-yl)methyl)piperidin-4-yl)isoindolin-2-yl)piperidine-2,6-dione;
3-(5-(1-((2-(tert-butyl)thiazol-4-yl)methyl)piperidin-4-yl)-1-oxoisoindolin-2-yl)piperidine-2,6-dione;
3-(1-oxo-5-(1-((2-(thiophen-2-yl)thiazol-5-yl)methyl)piperidin-4-yl)isoindolin-2-yl)piperidine-2,6-dione;
3-(5-(1-((5-cyclopropyl-1H-pyrazol-3-yl)methyl)piperidin-4-yl)-1-oxoisoindolin-2-yl)piperidine-2,6-dione;
3-(5-(1-((2-morpholinopyrimidin-5-yl)methyl)piperidin-4-yl)-1-oxoisoindolin-2--yl)piperidine-2,6-dione; 3-(1-oxo-5-(1-((3-phenyl-1H-pyrazol-4-yl)methyl)piperidin-4-yl)isoindolin-2-yl)piperidine-2,6-dione;
3-(5-(1-((6-methyl-1H-indol-3-yl)methyl)piperidin-4-yl)-1-oxoisoindolin-2-yl)piperidine-2,6-dione;
methyl 4-((4-(2-(2,6-dioxopiperidin-3-yl)-1-oxoisoindolin-5-yl)piperidin-1-yl)methyl)-1H-pyrrole-2-carboxylate;
3-(1-oxo-5-(1-((3-(pyridin-3-yl)-1H-pyrazol-4-yl)methyl)piperidin-4-yl)isoindolin-2-yl)piperidine-2,6-dione;
3-(1-oxo-5-(1-((5-(pyridin-2-yl)-1H-pyrazol-3-yl)methyl)piperidin-4-yl)isoindolin-2-yl)piperidine-2,6-dione;
3-(5-(1-(3,5-difluoro-4-hydroxybenzyl)piperidin-4-yl)-1-oxoisoindolin-2-yl)piperidine-2,6-dione;
3-(5-(1-(2-methylbenzyl)piperidin-4-yl)-1-oxoisoindolin-2-yl)piperidine-2,6-dione;
3-(5-(1-(4-methylbenzyl)piperidin-4-yl)-1-oxoisoindolin-2-yl)piperidine-2,6-dione;
3-(5-(1-(3,5-dimethylbenzyl)piperidin-4-yl)-1-oxoisoindolin-2-yl)piperidine-2,6-dione;
3-(5-((2S)-1-benzyl-2-methylpiperidin-4-yl)-1-oxoisoindolin-2-yl)piperidine-2,6-dione;
3-(5-((2R)-1-benzyl-2-methylpiperidin-4-yl)-1-oxoisoindolin-2-yl)piperidine-2,6-dione;
3-(5-(1-benzyl-2-methylpiperidin-4-yl)-1-oxoisoindolin-2-yl)piperidine-2,6-dione;
3-(5-(1-methyl-1,2,3,6-tetrahydropyridin-4-yl)-1-oxoisoindolin-2-yl)piperidine-2,6-dione;
3-(1-oxo-5-(1-((5,6,7,8-tetrahydronaphthalen-1-yl)methyl)piperidin-4-yl)isoindolin-2-yl)piperidine-2,6-dione;
3-(5-(azepan-4-yl)-1-oxoisoindolin-2-yl)piperidine-2,6-dione;
3-(5-((R)-azepan-4-yl)-1-oxoisoindolin-2-yl)piperidine-2,6-dione;
3-(5-((S)-azepan-4-yl)-1-oxoisoindolin-2-yl)piperidine-2,6-dione;
3-(1-oxo-5-(1-((1,2,3,4-tetrahydronaphthalen-1-yl)methyl)piperidin-4-yl)isoindolin-2-yl)piperidine-2,6-dione;
methyl 2-(4-(2-(2,6-dioxopiperidin-3-yl)-1-oxoisoindolin-5-yl)piperidin-1-yl)acetate;
3-(1-oxo-5-(1-phenylpiperidin-4-yl)isoindolin-2-yl)piperidine-2,6-dione;
3-(5-(1-(3-methylbenzyl)piperidin-4-yl)-1-oxoisoindolin-2-yl)piperidine-2,6-dione;
3-(5-(1-(2,6-dimethylbenzyl)piperidin-4-yl)-1-oxoisoindolin-2-yl)piperidine-2,6-dione;
3-(1-oxo-5-(1-((5,6,7,8-tetrahydronaphthalen-2-yl)methyl)piperidin-4-yl)isoindolin-2-yl)piperidine-2,6-dione;
ethyl 2-(4-(2-(2,6-dioxopiperidin-3-yl)-1-oxoisoindolin-5-yl)piperidin-1-yl)acetate;
tert-butyl 2-(4-(2-(2,6-dioxopiperidin-3-yl)-1-oxoisoindolin-5-yl)piperidin-1-yl)acetate;
2-(4-(2-(2,6-dioxopiperidin-3-yl)-1-oxoisoindolin-5-yl)piperidin-1-yl)acetic acid;
3-(1-oxo-5-(1-(3,3,3-trifluoropropyl)piperidin-4-yl)isoindolin-2-yl)piperidine-2,6-dione;
2-(4-(2-(2,6-dioxopiperidin-3-yl)-1-oxoisoindolin-5-yl)piperidin-1-yl)-N-phenylacetamide;
3-(5-(1-(3-fluoropropyl)piperidin-4-yl)-1-oxoisoindolin-2-yl)piperidine-2,6-dione;
tert-butyl 4-((4-(2-(2,6-dioxopiperidin-3-yl)-1-oxoisoindolin-5-yl)piperidin-1-yl)methyl)benzoate;
3-(5-(1-benzyl-3,3-dimethylpiperidin-4-yl)-1-oxoisoindolin-2-yl)piperidine-2,6-dione;
3-(5-(1-benzyl-3-methylpiperidin-4-yl)-1-oxoisoindolin-2-yl)piperidine-2,6-dione;
3-(5-(1-(2-hydroxy-1-phenylethyl)piperidin-4-yl)-1-oxoisoindolin-2-yl)piperidine-2,6-dione;
3-(5-((S)-1-benzylazepan-4-yl)-1-oxoisoindolin-2-yl)piperidine-2,6-dione;
3-(5-(1-benzyl-2,5-dihydro-1H-pyrrol-3-yl)-1-oxoisoindolin-2-yl)piperidine-2,6-dione;
3-(5-(1-benzyl-2-oxopiperidin-4-yl)-1-oxoisoindolin-2-yl)piperidine-2,6-dione;
3-(5-(1-benzyl-1,2,3,4-tetrahydroquinolin-4-yl)-1-oxoisoindolin-2-yl)piperidine-2,6-dione;
3-(5-(1-((1-benzyl-1H-tetrazol-5-yl)methyl)piperidin-4-yl)-1-oxoisoindolin-2-yl)piperidine-2,6-dione;
3-(1-oxo-5-(1-((5-phenyl-1,3,4-oxadiazol-2-yl)methyl)piperidin-4-yl)isoindolin-2-yl)piperidine-2,6-dione;
3-(5-(1-(benzo[d]thiazol-2-ylmethyl)piperidin-4-yl)-1-oxoisoindolin-2-yl)piperidine-2,6-dione;
3-(1-oxo-5-(1-((3-(pyridin-2-yl)-1H-pyrazol-5-yl)methyl)piperidin-4-yl)isoindolin-2-yl)piperidine-2,6-dione;
3-(5-(1-((R)-2-hydroxy-1-phenylethyl)piperidin-4-yl)-1-oxoisoindolin-2-yl)piperidine-2,6-dione;
3-(5-(1-((1-methyl-1H-indazol-3-yl)methyl)piperidin-4-yl)-1-oxoisoindolin-2-yl)piperidine-2,6-dione;
3-(5-(1-((1,2,4-oxadiazol-3-yl)methyl)piperidin-4-yl)-1-oxoisoindolin-2-yl)piperidine-2,6-dione;
3-(5-(1-(4-hydroxy-3-((4-methylpiperazin-1-yl)methyl)benzyl)piperidin-4-yl)-1-oxoisoindolin-2-yl)piperidine-2,6-dione;
2-(4-((4-(2-(2,6-dioxopiperidin-3-yl)-1-oxoisoindolin-5-yl)piperidin-1-yl)methyl)phenyl)acetonitrile;
3-(5-(1-((7-hydroxy-2-methylpyrazolo[1,5-a]pyrimidin-5-yl)methyl)piperidin-4-yl)-1-oxoisoindolin-2-yl)piperidine-2,6-dione;
3-(5-(1-(2,2-difluoro-1-phenylethyl)piperidin-4-yl)-1-oxoisoindolin-2-yl)piperidine-2,6-dione;
3-(5-(1-((3-fluorobicyclo[1.1.1]pentan-1-yl)methyl)piperidin-4-yl)-1-oxoisoindolin-2-yl)piperidine-2,6-dione;
3-(5-(1-(2-fluoro-1-phenylethyl)piperidin-4-yl)-1-oxoisoindolin-2-yl)piperidine-2,6-dione;
3-(1-oxo-5-(1-((4-oxo-3,4-dihydrothieno[3,2-d]pyrimidin-2-yl)methyl)piperidin-4-yl)isoindolin-2-yl)piperidine-2,6-dione;
3-(1-oxo-5-(1-(quinolin-4-ylmethyl)piperidin-4-yl)isoindolin-2-yl)piperidine-2,6-dione;
3-(5-(1-(3,5-bis(trifluoromethyl)benzyl)piperidin-4-yl)-1-oxoisoindolin-2-yl)piperidine-2,6-dione; 6-((4-(2-(2,6-dioxopiperidin-3-yl)-1-oxoisoindolin-5-yl)piperidin-1-yl)methyl)picolinonitrile;
2-(4-((4-(2-(2,6-dioxopiperidin-3-yl)-1-oxoisoindolin-5-yl)piperidin-1-yl)methyl)phenoxy)acetonitrile;
3-(5-(1-((1H-indazol-5-yl)methyl)piperidin-4-yl)-1-oxoisoindolin-2-yl)piperidine-2,6-dione;
3-(5-(1-(2,2-difluoroethyl)piperidin-4-yl)-1-oxoisoindolin-2-yl)piperidine-2,6-dione;
3-(5-(1-((7-methyl-4-oxo-4H-pyrido[1,2-a]pyrimidin-2-yl)methyl)piperidin-4-yl)-1-oxoisoindolin-2-yl)piperidine-2,6-dione;
benzyl 4-(2-(2,6-dioxopiperidin-3-yl)-1-oxoisoindolin-5-yl)piperidine-1-carboxylate;
3-(1-oxo-5-(1-(2-phenylacetyl)piperidin-4-yl)isoindolin-2-yl)piperidine-2,6-dione;
3-(1-oxo-5-(1-(2,2,2-trifluoro-1-phenylethyl)piperidin-4-yl)isoindolin-2-yl)piperidine-2,6-dione;
3-(5-(1-(4-(5-methylbenzo[d]thiazol-2-yl)benzyl)piperidin-4-yl)-1-oxoisoindolin-2-yl)piperidine-2,6-dione;
3-(5-(1-(isoquinolin-1-ylmethyl)piperidin-4-yl)-1-oxoisoindolin-2-yl)piperidine-2,6-dione;
3-(5-(1-(4-(4-methoxypiperidin-1-yl)benzyl)piperidin-4-yl)-1-oxoisoindolin-2-yl)piperidine-2,6-dione;
3-(5-(1-(4-(isopropylthio)benzyl)piperidin-4-yl)-1-oxoisoindolin-2-yl)piperidine-2,6-dione;
3-(1-oxo-5-(1-((S)-1-phenylethyl)piperidin-4-yl)isoindolin-2-yl)piperidine-2,6-dione;
2-(4-((4-(2-(2,6-dioxopiperidin-3-yl)-1-oxoisoindolin-5-yl)piperidin-1-yl)methyl)phenyl)acetic acid;
3-(5-(1-((7-fluoroquinolin-2-yl)methyl)piperidin-4-yl)-1-oxoisoindolin-2-yl)piperidine-2,6-dione;
3-(5-(1-((2-amino-4-(trifluoromethyl)thiazol-5-yl)methyl)piperidin-4-yl)-1-oxoisoindolin-2-yl)piperidine-2,6-dione;
3-((4-(2-(2,6-dioxopiperidin-3-yl)-1-oxoisoindolin-5-yl)piperidin-1-yl)methyl)-1,2,4-oxadiazole-5-carboxamide;
3-(5-(1-(3-(morpholinosulfonyl)benzyl)piperidin-4-yl)-1-oxoisoindolin-2-yl)piperidine-2,6-dione;
4-((4-(2-(2,6-dioxopiperidin-3-yl)-1-oxoisoindolin-5-yl)piperidin-1-yl)methyl)-N,N-dimethylbenzenesulfonamide;
3-(1-oxo-5-(1-(thiazol-4-ylmethyl)piperidin-4-yl)isoindolin-2-yl)piperidine-2,6-dione;
3-(1-oxo-5-(1-(quinoxalin-6-ylmethyl)piperidin-4-yl)isoindolin-2-yl)piperidine-2,6-dione;
3-(5-(1-(4-(tert-butyl)benzoyl)piperidin-4-yl)-1-oxoisoindolin-2-yl)piperidine-2,6-dione;
3-(5-(1-(4-((4-fluorobenzyl)oxy)benzyl)piperidin-4-yl)-1-oxoisoindolin-2-yl)piperidine-2,6-dione;
3-(5-(1-((3-methylisoxazol-5-yl)methyl)piperidin-4-yl)-1-oxoisoindolin-2-yl)piperidine-2,6-dione;
3-(5-(1-(isoxazol-3-ylmethyl)piperidin-4-yl)-1-oxoisoindolin-2-yl)piperidine-2,6-dione;
3-(1-oxo-5-(1-((R)-1-phenylethyl)piperidin-4-yl)isoindolin-2-yl)piperidine-2,6-dione;
3-(5-(1-(4-(methoxymethyl)benzyl)piperidin-4-yl)-1-oxoisoindolin-2-yl)piperidine-2,6-dione;
3-(5-(1-((S)-2-hydroxy-1-phenylethyl)piperidin-4-yl)-1-oxoisoindolin-2-yl)piperidine-2,6-dione;
3-(1-oxo-5-(1-(phenylsulfonyl)piperidin-4-yl)isoindolin-2-yl)piperidine-2,6-dione;
3-(5-(1-((5-methyl-3-phenylisoxazol-4-yl)methyl)piperidin-4-yl)-1-oxoisoindolin-2-yl)piperidine-2,6-dione;
3-(5-(1-(4-((difluoromethyl)sulfonyl)benzyl)piperidin-4-yl)-1-oxoisoindolin-2-yl)piperidine-2,6-dione;
3-(1-oxo-5-(1-(2,2,2-trifluoroethyl)piperidin-4-yl)isoindolin-2-yl)piperidine-2,6-dione;
methyl 2-((4-(2-(2,6-dioxopiperidin-3-yl)-1-oxoisoindolin-5-yl)piperidin-1-yl)methyl)oxazole-4-carboxylate;
3-(1-oxo-5-(1-(4-(pyridin-2-ylmethoxy)benzyl)piperidin-4-yl)isoindolin-2-yl)piperidine-2,6-dione;
3-(5-(1-acetylpiperidin-4-yl)-1-oxoisoindolin-2-yl)piperidine-2,6-dione;
3-(1-oxo-5-(1-((2-oxo-2,3-dihydro-1H-benzo[d]imidazol-5-yl)methyl)piperidin-4-yl)isoindolin-2-yl)piperidine-2,6-dione;
3-(5-(1-benzylpyrrolidin-3-yl)-1-oxoisoindolin-2-yl)piperidine-2,6-dione;
(R)-3-(5-((R)-1-benzylazepan-4-yl)-1-oxoisoindolin-2-yl)piperidine-2,6-dione;
(S)-3-(5-((S)-1-benzylazepan-4-yl)-1-oxoisoindolin-2-yl)piperidine-2,6-dione;
3-(5-(1-benzylazepan-4-yl)-1-oxoisoindolin-2-yl)piperidine-2,6-dione;
3-(5-(1-methyl-2,3,6,7-tetrahydro-1H-azepin-4-yl)-1-oxoisoindolin-2-yl)piperidine-2,6-dione;
3-(5-(8-benzyl-8-azabicyclo[3.2.1]octan-3-yl)-1-oxoisoindolin-2-yl)piperidine-2,6-dione;
3-(5-(8-azabicyclo[3.2.1]octan-3-yl)-1-oxoisoindolin-2-yl)piperidine-2,6-dione;
3-(5-(1-acetyl-1,2,5,6-tetrahydropyridin-3-yl)-1-oxoisoindolin-2-yl)piperidine-2,6-dione;
(R)-3-(5-((R)-1-acetylpyrrolidin-3-yl)-1-oxoisoindolin-2-yl)piperidine-2,6-dione;
3-(5-(1-acetyl-1,2,3,6-tetrahydropyridin-4-yl)-1-oxoisoindolin-2-yl)piperidine-2,6-dione;
3-(5-(octahydroindolizin-7-yl)-1-oxoisoindolin-2-yl)piperidine-2,6-dione;
(R)-3-(5-((S)-1-benzylazepan-4-yl)-1-oxoisoindolin-2-yl)piperidine-2,6-dione;
3-(5-((R)-1-benzylazepan-4-yl)-1-oxoisoindolin-2-yl)piperidine-2,6-dione;
3-(5-(1-acetyl-2,5-dihydro-1H-pyrrol-3-yl)-1-oxoisoindolin-2-yl)piperidine-2,6-dione;
3-(5-(1-methylazepan-4-yl)-1-oxoisoindolin-2-yl)piperidine-2,6-dione;
(S)-3-(5-((R)-1-benzylazepan-4-yl)-1-oxoisoindolin-2-yl)piperidine-2,6-dione;
(S)-3-(5-((R)-1-acetylpyrrolidin-3-yl)-1-oxoisoindolin-2-yl)piperidine-2,6-dione;
3-(5-(1-((6-isopropoxypyridin-3-yl)methyl)piperidin-4-yl)-1-oxoisoindolin-2-yl)piperidine-2,6-dione;
3-(1-oxo-5-(1-((1-phenyl-1H-pyrazol-5-yl)methyl)piperidin-4-yl)isoindolin-2-yl)piperidine-2,6-dione;
3-(5-(1-(4-ethoxybenzyl)piperidin-4-yl)-1-oxoisoindolin-2-yl)piperidine-2,6-dione;
3-(1-oxo-5-(1-((1-phenyl-1H-pyrazol-4-yl)methyl)piperidin-4-yl)isoindolin-2-yl)piperidine-2,6-dione;
3-(5-(1-((1-isopropyl-1H-pyrazol-5-yl)methyl)piperidin-4-yl)-1-oxoisoindolin-2-yl)piperidine-2,6-dione;
3-(5-(1-(isothiazol-5-ylmethyl)piperidin-4-yl)-1-oxoisoindolin-2-yl)piperidine-2,6-dione;
3-(5-(1-((1-isopropyl-1H-pyrazol-4-yl)methyl)piperidin-4-yl)-1-oxoisoindolin-2-yl)piperidine-2,6-dione;
3-(5-(1-((1H-pyrazol-5-yl)methyl)piperidin-4-yl)-1-oxoisoindolin-2-yl)piperidine-2,6-dione;
3-(5-(1-((5-isopropoxypyridin-2-yl)methyl)piperidin-4-yl)-1-oxoisoindolin-2-yl)piperidine-2,6-dione;
3-(1-oxo-5-(1-((1-(pyridin-3-yl)-1H-pyrazol-5-yl)methyl)piperidin-4-yl)isoindolin-2-yl)piperidine-2,6-dione;
3-(1-oxo-5-(1-((1-(pyridin-3-yl)-1H-pyrazol-4-yl)methyl)piperidin-4-yl)isoindolin-2-yl)piperidine-2,6-dione;
5-((4-(2-(2,6-dioxopiperidin-3-yl)-1-oxoisoindolin-5-yl)piperidin-1-yl)methyl)-2-fluorobenzonitrile;
3-(5-(1-((5-fluoropyridin-2-yl)methyl)piperidin-4-yl)-1-oxoisoindolin-2-yl)piperidine-2,6-dione;
3-(5-(1-((1-ethyl-3-(pyridin-3-yl)-1H-pyrazol-4-yl)methyl)piperidin-4-yl)-1-oxoisoindolin-2-yl)piperidine-2,6-dione;
trans-3-(1-oxo-5-(1-((4-(trifluoromethyl)cyclohexyl)methyl)piperidin-4-yl)isoindolin-2-yl)piperidine-2,6-dione;
cis-3-(1-oxo-5-(1-((4-(trifluoromethyl)cyclohexyl)methyl)piperidin-4-yl)isoindolin-2-yl)piperidine-2,6-dione;
trans-3-(5-(1-((4-methoxycyclohexyl)methyl)piperidin-4-yl)-1-oxoisoindolin-2-yl)piperidine-2,6-dione; and
3-(5-(1-((6-methoxypyridin-2-yl)methyl)piperidin-4-yl)-1-oxoisoindolin-2-yl)piperidine-2,6-dione;
   or a pharmaceutically acceptable salt, hydrate, solvate, prodrug, stereoisomer, or tautomer thereof.

Additional exemplary IKZF2 inhibitors are also described in U.S. Pat. Nos. 10,414,755 and 10,640,489, U.S. Application Publication Nos. US2019/0367483 and US2019/0359594, and International Publication Nos. WO2020/012334 and WO2019/038717, which are incorporated by reference in their entirety.

In some embodiments, the ZBTB32 inhibitor and the IKZF2 inhibitor are used in combination to treat a non-small cell lung cancer (NSCLC), a melanoma, a triple-negative breast cancer (TNBC), nasopharyngeal cancer (NPC), a microsatellite stable colorectal cancer (mssCRC), a thymoma, a carcinoid, or gastrointestinal stromal tumor (GIST).

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present disclosure and practice the claimed methods. The following working examples specifically point out various embodiments of the present disclosure, and are not to be construed as limiting in any way the remainder of the disclosure.

### Example 1: Expression of ZBTB32 in Cancer Cell Lines

This Example describes ZBTB32 expression in cancer cell lines.

ZBTB32 gene expression data in a variety of cancer cell lines was obtained from the Cancer Cell Line Encyclopedia (CCLE) database (Ghandi, M et al. Nature, 2019, 569:503-508). FIG. 1 shows ZBTB32 expression in the specified cell lines from RNAseq analysis. As shown in FIG. 1 and Table 27, cells expressing high level of ZBTB32 are mainly from hematopoietic and lymphoid tissue and in lymphoma, multiple myeloma and leukemia lineages.

TABLE 27
List of cell lines expressing ZBTB32 at the level above 5 transcripts per million (TPM).

| | | | ZBTB32 |
|---|---|---|---|
| | | | expression |
| Cell line | Primary site | Lineage | (TPM) |
| JVM2 | haematopoietic_and_lymphoid_tissue | Lymphoma: NH_B_cell | 290.205 |
| EHEB | haematopoietic_and_lymphoid_tissue | Leukemia: CLL | 144.784 |
| JVM3 | haematopoietic_and_lymphoid_tissue | Leukemia: CLL | 126.08 |
| REC1 | haematopoietic_and_lymphoid_tissue | Lymphoma: NH_B_cell | 115.949 |
| HUNS1 | haematopoietic_and_lymphoid_tissue | Multiple_Myeloma | 104.362 |
| SUPM2 | haematopoietic_and_lymphoid_tissue | Lymphoma: NH_T_cell | 75.58 |
| CI1 | haematopoietic_and_lymphoid_tissue | Lymphoma: NH_B_cell | 68.651 |
| KE97 | haematopoietic_and_lymphoid_tissue | Multiple_Myeloma | 62.521 |
| HH | haematopoietic_and_lymphoid_tissue | Leukemia: ALL | 62.373 |
| MEC1 | haematopoietic_and_lymphoid_tissue | Leukemia: CLL | 57.476 |
| MINO | haematopoietic_and_lymphoid_tissue | Lymphoma: NH_B_cell | 52.081 |
| AMO1 | haematopoietic_and_lymphoid_tissue | Multiple_Myeloma | 47.682 |
| SUDHL1 | haematopoietic_and_lymphoid_tissue | Lymphoma: NH_T_cell | 46.951 |
| MOLP2 | haematopoietic_and_lymphoid_tissue | Multiple_Myeloma | 46.313 |
| KIJK | haematopoietic_and_lymphoid_tissue | Lymphoma: NH_T_cell | 44.024 |
| KMH2 | haematopoietic_and_lymphoid_tissue | Lymphoma: Hodgkin | 43.974 |
| BDCM | haematopoietic_and_lymphoid_tissue | Leukemia: ALL | 40.659 |
| EB1 | haematopoietic_and_lymphoid_tissue | Lymphoma: NH_B_cell | 37.22 |
| A3KAW | haematopoietic_and_lymphoid_tissue | Lymphoma: NH_B_cell | 34.296 |
| HDLM2 | haematopoietic_and_lymphoid_tissue | Lymphoma: Hodgkin | 33.046 |
| L540 | haematopoietic_and_lymphoid_tissue | Lymphoma: Hodgkin | 31.756 |
| HUT102 | haematopoietic_and_lymphoid_tissue | Lymphoma: NH_T_cell | 29.964 |
| RAJI | haematopoietic_and_lymphoid_tissue | Lymphoma: NH_B_cell | 29.716 |
| L1236 | haematopoietic_and_lymphoid_tissue | Lymphoma: Hodgkin | 29.105 |
| MJ | haematopoietic_and_lymphoid_tissue | Lymphoma: NH_T_cell | 26.968 |
| KARPAS299 | haematopoietic_and_lymphoid_tissue | Lymphoma: NH_T_cell | 21.63 |
| SR786 | haematopoietic_and_lymphoid_tissue | Lymphoma: NH_T_cell | 21.568 |
| GRANTA519 | haematopoietic_and_lymphoid_tissue | Lymphoma: NH_B_cell | 19.375 |
| A4FUK | haematopoietic_and_lymphoid_tissue | Lymphoma: NH_B_cell | 16.359 |
| HS611T | haematopoietic_and_lymphoid_tissue | Lymphoma: Hodgkin | 14.464 |
| VMCUB1 | urinary_tract | Bladder: Carcinoma | 14.303 |
| JEKO1 | haematopoietic_and_lymphoid_tissue | Lymphoma: NH_B_cell | 13.751 |
| NUDHL1 | haematopoietic_and_lymphoid_tissue | Lymphoma: NH_B_cell | 13.246 |
| L428 | haematopoietic_and_lymphoid_tissue | Lymphoma: Hodgkin | 12.78 |
| NCIH854 | lung | Lung: NSCLC_Adeno | 12.137 |
| LS123 | large_intestine | Colorectal: Carcinoma | 11.889 |
| HEC108 | endometrium | Endometrium: Carcinoma | 10.45 |
| DEL | haematopoietic_and_lymphoid_tissue | Lymphoma: NH_T_cell | 10.141 |
| SNU719 | stomach | Gastric: Carcinoma | 10.113 |
| OCILY3 | haematopoietic_and_lymphoid_tissue | Lymphoma: NH_B_cell | 8.488 |
| SNU410 | pancreas | Pancreas: Carcinoma | 7.862 |
| BFTC905 | urinary_tract | Bladder: Carcinoma | 7.574 |
| MDAMB134VI | breast | Breast: Carcinoma | 7.131 |
| NCIH522 | lung | Lung: NSCLC_Adeno | 6.954 |
| LC1F | lung | Lung: NSCLC_Squamous | 6.347 |
| NCIH1355 | lung | Lung: NSCLC_Adeno | 5.994 |
| NCIH727 | lung | Endocrine | 5.908 |
| SW837 | large_intestine | Colorectal: Carcinoma | 5.876 |
| SBC5 | lung | Lung: SCLC | 5.564 |
| RERFGC1B | stomach | Gastric: Carcinoma | 5.395 |
| GCIY | stomach | Gastric: Carcinoma | 5.387 |
| DMS114 | lung | Lung: SCLC | 5.288 |
| MORCPR | lung | Lung: NSCLC_Adeno | 5.102 |
| DAUDI | haematopoietic_and_lymphoid_tissue | Lymphoma: NH_B_cell | 5.055 |

It has been shown that ZBTB32 is one the most consistently and differentially expressed genes in activated B cell subtype ofDLBCL (ABC-DLBCL) patients compared to other DLBCL types (Care et al. PLoS One. 2013; 8(2):e55895). Accordingly, RNA sequencing analysis showed much higher levels of ZBTB32 expression in ABC-DLBCL than germinal center B cell DLBCL (GCB-DLBCL) cell lines (Table 28).

TABLE 28
ZBTB32 Expression in ABC- and GCB-type of DLBCL Cell lines. CPM: counts per million reads

| | Type | | | | | | | | |
|---|---|---|---|---|---|---|---|---|---|
| | ABC | | | | | GCB | | | |
| Cell line | RC-K8 | HBL-1 | TMD-8 | OCI-Ly10 | OCI-Ly3 | Su-DHL10 | Su-DHL5 | Su-DHL4 | Su-DHL6 |
| ZBTB32 | 7.21 | 7.90 | 7.21 | 5.49 | 5.83 | −1.42 | 1.33 | −1.38 | −0.32 |
| expression | | | | | | | | | |
| (Log2CPM) | | | | | | | | | |

### Example 2: Enhanced CART Activity with ZBTB32 Knockout

This Example describes enhanced CART cell expansion, cytokine production, persistence, resistance to exhaustion and anti-tumor activity in vivo when ZBTB32 is knocked out in the CART cells.

### Material and Methods

### CART Generation

Frozen T cells previously isolated from fresh blood were thawed and activated with CD3/CD28 beads (Thermofisher) at a 1:3 cell to bead ratio on day 0. The following day, 10×10⁶ cells were plated in 6-well plate in 5 mL of T cell media without antibiotics and GFP (lacking CAR coding sequence) or CAR19-GFP virus was added dropwise (MOI=1). On day 3, CRISPR-Cas9 electroporation was performed to knock out ZBTB32 in the CAR19-GFP transduced cells as below. Denature RNA secondary structure by heating trRNA and gRNA in separate tubes to 98° C. for 2 minutes, and anneal by gradually decreasing temperature to 38° C. In a separate tube, create the RNP mixture by adding Cas9 buffer, Cas9 protein, trRNA and gRNA in order and mixing well after every addition. The final ratio of Cas9:tracerRNA:gRNA is 1:2:2. Incubate the samples at 37° C. for 10 minutes while preparing the cells. Collect T cells, spin down, wash with warm OPTI-MEM, spin down, and resuspend in T buffer (Neon Transfection System) at 20×10⁶ cells/ml. Mix 100 uL of T cells with 50 uL of RNP mix and incubate 5 minutes at room temperature. Using the Neon machine, cells were electroporated using the settings of 1600V, 1Oms, 3 pulses, and transferred to 6-well plates with complete T cell media. ZBTB32 gRNA6 sequence: CCAGCCGATCAGAGCCATAG (SEQ ID NO: 3004). ZBTB32 gRNA7 sequence: GCTCTGGAGCCAGAACCAGT (SEQ ID NO: 3005). From day 4 onwards, cells were expanded by adding fresh media every other day. Cells were frozen down using CS10 freezing media when cell size reached 400 fm.

### Next-Generation Sequencing (NGS) and T7 Endonuclease I (T7E1) Assay

Cells were harvested prior to freezing down and genomic DNA was isolated using the PureLink Genomic DNA Mini Kit (Invitrogen). DNA fragment around the editing site was amplified by PCR using the Q5 high fidelity master mix (NEB). ZBTB32 gRNA6 NGS F: 5′-CCACAGGCTTGAAATGAGATG-3′ (SEQ ID NO: 3006), ZBTB32 gRNA6 NGS R: 5′-CATACACAAAGTTCAGGAGC-3′ (SEQ ID NO: 3007), ZBTB32 gRNA7 NGS F: 5′-GACAAGAAGCTGCTGCCACA-3′ (SEQ ID NO: 3008), ZBTB32 gRNA7 NGS R: 5′-GGAACCTGGAAGGAACAGTG-3′ (SEQ ID NO: 3009). The PCR product was purified using the Qiagen PCR purification kit and submitted for NGS. To perform the T7E1 assay, DNA fragment around the editing site was amplified by PCR using the Q5 high fidelity master mix (NEB). ZBTB32 gRNA6 T7 F: 5′-ATTTCCATTCTCTCGCCACC-3′ (SEQ ID NO: 3010), ZBTB32 gRNA6 T7R: 5′-CTGGGCAAAGGTAGAAGGGC-3′ (SEQ ID NO: 3011), ZBTB32 gRNA7 T7 F: 5′-GGTCTAGTGCTCTGCCTCCA-3′ (SEQ ID NO: 3012), ZBTB32 gRNA7 T7R: 5′-GCGCCAACTCACTGTACCATT-3′ (SEQ ID NO: 3013). PCR product was purified and 200ng of purified DNA were denatured and slowly annealed using the 10×NEBuffer2. The T7 Endonuclease I (NEB) was added to the annealed PCR product incubated for 15 minutes at 37° C. No T7E1 enzyme was added for uncut controls. An electrophoresis gel was run with cut and uncut samples to determine if editing occurred.

### Antigen-Dependent Proliferation Assay

TMD8 cells were irradiated at 10,000 rad and 50,000 irradiated cells were plated in 100 ul of T cell media in a 96-well plate. GFP (non-CAR) T cells or CART cells were thawed, serially diluted for various effector to target cell (E:T) ratios (10:1, 5:1, 2.5:1, 1.25:1, 0.625:1 and 0.3125:1), and plated in 100 ul of T cell media on top of the irradiated TMD8 cells. Cells were incubated for 3 and 5 days and analyzed by flow cytometry using the live/dead, CD3, CD4, CD8 and GFP staining. GFP % is used to determine antigen-dependent proliferation kinetics.

### Antigen-Dependent Killing Assay

TMD8-luciferase cells were plated in 100 ul of T cell media in a 96-well plate (25,000/well). CART cells were thawed, serially diluted for various E:T ratios (10:1, 5:1, 2.5:1, 1.25:1, 0.625:1 and 0.3125:1), and plated in 100 ul of T cell media on top of the TMD8-luciferase cells. Cells were incubated for 48 and 72 hours. At each time point, 100 ul of media was collected for MSD according to manufacturer's instructions, and 100 ul of Bright Glo reagent (Promega) was added and incubated for 10 minutes at room temperature prior to reading luminescence. TMD8-luciferase cells alone was used as the 0% killing control.

### In Vivo Efficacy Study Using TMD8 Model

TMD8 cells were thawed and cultured exponentially in house, and collected for implant on day 0. At time of harvest, cells were washed and re-suspended in a 1:1 ratio of PBS without CaCl₂ or MgCl₂ and matrigel to a concentration of 5×10⁶ cells per 100 μl for implant. Cells were implanted subcutaneously in the right flank of immunocompromised NSG mice (Jackson Laboratory). Baseline body weight was also recorded at time of implant. On day 9, tumors were calipered and randomized based on tumor volume. The average tumor volume was 178.3 mm³. CART infusion was administered on day 10. GFP wt (non-CAR) T cells and CART cells were thawed and washed in RPMI 1640+10% Heat Inactivated Fetal Bovine Serum (FBS). Cells were counted on a Cellometer Vision (Nexcelcom), and washed again prior to being re-suspended in PBS for injection. Cells were re-suspended to a final concentration of 1×10⁶ CAR+ cells per 100 μl of PBS based on the percent of GFP positivity of each sample. In vivo injection was delivered intravenously in the tail vein of the mice. One group of mice received a 100 μl PBS injection as a negative control. Tumors volume and body weight were monitored 2-3 times per week thereafter.

Blood was collected via lateral tail vein puncture starting 2 weeks post CART infusion, and continued thereafter through week 6. At each time point, approximately 50 μl of whole blood was collected into EDTA coated capillary tubes. 30 μl of blood was plated to be stained for flow cytometry, and the remaining blood was spun down for 5 minutes at 13,000 rpm. Plasma was

Extracted from Tubes, Plated, and Stored at −80° C. for Cytokine Evaluation.

In Vivo efficacy study using TMD8-Luciferase model Six-week old female NSG mice were received from the Jackson Laboratory. Electronic transponders for animal identification were implanted on the left flank one day prior to tumor implantation. TMD8-Luc cells were cultured with RMPI-1640 medium containing 10% fetal bovine serum and 1% L-glutamine. Cells in logarithmic growth phase were harvested and washed once in cold sterile PBS. Cells were resuspended in a 1:1 ratio of PBS and matrigel at a concentration of 25×10⁶ per ml, placed on ice, and subcutaneously injected into the right flank of mice at 200 ul. Total cell number per mouse was 5×10⁶. Nine days after tumor implantation, mice were randomized. Wild type or ZBTB32KO gRNA6 CAR T cells were thawed, washed with RPMI 1640+10% Heat Inactivated Fetal Bovine Serum (FBS), and counted by a Cellometer Vision (Nexcelcom). CART cells centrifuged at 300 g for 10 minutes and resuspended at respective concentrations in cold PBS and kept on ice until injection. Mice were injected intravenously via the tail vein with 200 μl PBS (vehicle) or 200 ul PBS containing 2×10⁶ or 0.4×10⁶ wild type or ZBTB32KO gRNA6 CAR T cells. Tumors and body weight were monitored 2-3 times per week thereafter. TMD8-Luc cells express Luciferase. To measure the Luciferase signal, mice were IP injected with 15 mg/ml of luciferin substrate in a 200 ul dose and images were taken 10 minutes later for quantification.

### Flow Cytometry Analysis

Blood samples were transferred into 96-well U bottom plate (10-30 ul blood for each panel) and red blood cells were lysed by adding 150 ul per well of ACK lysing buffer (Quality Biological, Cat #118-156-721). After 2 minutes incubation at room temperature, 100 ul of Dulbecco's phosphate-buffered saline (DPBS) was added to each well, cells were spun down at 1500 rpm for 5 minutes and washed 200 ul DPBS. For cell surface and transcription factor panels, cells were directly processed to the staining procedures described below. For cytokine panel, to stimulate cytokine expression, cells were resuspended in 200 ul T cell culture media (RPMI1640+10% FBS+1× Sodium Pyruvate+1×NEAA+1× L-Glutamine+1× HEPES+1× 2-Mercaptoethanol+1×Penicillin/Streptomycin) containing PMA (Sigma, final concentration: 10 ng/ml) and Ionomycin (Sigma, final concentration: 1 ug/ml) and incubated at 37° C. incubator for 3 hours. Brefeldin A (BioLegend, final concentration: 10 ug/mL) was added to each well and cells were further incubated at 37° C. incubator for 1 hour. Cells were spun down at 1500 rpm for 5 minutes and washed with 200 ul DPBS.

Cells were then resuspended with 100 ul Zombie NIR in DPBS (1:1500). After 15 minutes incubation at room temperature in dark, cells were spinned down at 1800 rpm for 3 minutes and washed with FACS buffer (1000 mL DPBS+40 mL heat inactivated FBS+1 mL 0.5M EDTA pH8.0).

For cell surface staining, cells were resuspended with 50 ul FACS buffer containing mouse Fc blocking ab (Miltenyi, Cat #130-092-575, 1:25 dilution) and human Fc blocking ab (BD, Cat #564220, 1:10 dilution) and incubated at room temperature in dark for 20 minutes. Then 50 ul of 2×surface antibodies mixture in FACS buffer was added, mixed and incubated at room temperature in dark for 30 minutes. Cells were washed twice with FACS buffer and analyzed using BD Fortessa flow cytometer.

For intracellular staining, cells were resuspended with 200 ul 1× fixation/permeablization buffer (Invitrogen, Cat #00-5523-00), mixed and incubate at room temperature in dark for 30 minutes. Cells were washed twice with 200 ul 1× permeablization buffer, resuspended with 100 ul 1× intracellular antibodies mixture in 1× permeablization buffer and incubate at room temperature in dark for 30 minutes. Cells were washed with 200 ul 1× permeablization buffer and fixed with 200 uL 0.05 paraformaldehyde in DPBS at room temperature for 30 minutes. After fixation, cells were spinned down to remove paraformaldehyde and resuspended in 200 uL FACS buffer to be analyzed using BD Fortessa flow cytometer.

TABLE 29
List of flow antibodies

| | Color | Vendor | Clone | Cat# | Dilution |
|---|---|---|---|---|---|
| Surface | | | | | |
| L/D (Zombie | APC/Cy7 | Biolegend | | 423106 | 1:1500 |
| NIR) | | | | | |
| mCD11b | PerCP-Cy5.5 | BioLegend | M1/70 | 101228 | 1:100 |
| CD45 | APC | BioLegend | 2D1 | 368512 | 1:100 |
| CD3 | BV510 | BioLegend | OKT3 | 317332 | 1:50 |
| CD4 | BUV737 | BD | SK3 | 612748 | 1:100 |
| CD8 | BUV395 | BD | RPA-T8 | 563795 | 1:100 |
| TIGIT | BV421 | BioLegend | A15153G | 372710 | 1:100 |
| PD1 | PE-Dazzle594 | BioLegend | EH12.2H7 | 329940 | 1:100 |
| TIM3 | BV785 | BioLegend | F38-2E2 | 345032 | 1:100 |
| LAG3 | PE/Cy7 | BioLegend | 7H2C65 | 369208 | 1:100 |
| Transcription | | | | | |
| factors | | | | | |
| L/D (Zombie | APC/Cy7 | Biolegend | | 423106 | 1:1500 |
| NIR) | | | | | |
| CD45 | BV785 | BD | HI30 | 563716 | 1:100 |
| CD3 | BV510 | BioLegend | OKT3 | 317332 | 1:50 |
| CD4 | BUV737 | BD | SK3 | 612748 | 1:100 |
| CD8 | BUV395 | BD | RPA-T8 | 563795 | 1:100 |
| Eomes | PE/Cy7 | Thermo | WD1928 | 25-4877- | 1:100 |
| | | | | 42 | |
| Surface | | | | | |
| Tcf7 | AF647 | BioLegend | 7F11A10 | 655204 | 1:50 |
| Tox | PE | Miltenyi | REA473 | 130-120- | 1:100 |
| | | | | 716 | |
| Tbet | BV711 | BioLegend | 4B10 | 644820 | 1:100 |
| Cytokines | | | | | |
| L/D (Zombie | APC/Cy7 | Biolegend | | 423106 | 1:1500 |
| NIR) | | | | | |
| CD45 | BV785 | BD | HI30 | 563716 | 1:100 |
| CD3 | BV510 | BioLegend | OKT3 | 317332 | 1:50 |
| CD4 | BUV737 | BD | SK3 | 612748 | 1:100 |
| CD8 | BUV395 | BD | RPA-T8 | 563795 | 1:100 |
| IL2 | BV421 | BioLegend | MAQ1- | 500328 | 1:100 |
| | | | 17H12 | | |
| IFNg | PE/Cy7 | BioLegend | 4S.B3 | 502528 | 1:100 |
| TNFa | PerCP/Cy5.5 | BioLegend | MAb11 | 502926 | 1:100 |
| GranzB | APC | BioLegend | QA16A02 | 372204 | 1:100 |
| GranzK | AF594 | BioLegend | GM26E7 | 370506 | 1:100 |

CART Isolation from Spleens

At endpoint (day 53), all remaining 14-15 tumor free mice were sacrificed and their spleens were collected. Spleens were smashed through a 70 μm filter into −10 ml media (RPMI+10% FBS) in a 50 ml conical tube. Filter was washed with media to ensure that spleen cells are collected into the conical tube. Four or 5 spleens were pooled to one. Collection tubes were spun at 1500 rpm for 5 min, cell pallet was resuspended with 1 ml of ACK lysing buffer (Quality Biological, Cat #118-156-721) and incubated at room temperature for 2 minutes. Cells were washed with DPBS, stained with live/dead and processed to sort out GFP positive live cells using FACSAria sorter.

### Statistical Analyses

One-way ANOVA and unpaired t-test were performed to determine statistical differences using GraphPad Prism software.

### Results

To investigate the role of ZBTB32 in human T cell mediated anti-tumor immune responses, wild type (wt) and ZBTB32 KO CAR19-GFP T cells were generated. The CAR positivity was determined by GFP and anti-CD19 CAR idiotype antibody staining flow cytometry analysis (FIG. 2A) and the gene editing efficiency was verified by T7E1 cutting assay and NGS (FIGS. 2B-2C). As shown in FIG. 2A, CAR19 is expressed at similar levels in ZBTB32 knockout cells as compared to wild type cells with no ZBTB32 disruption. ZBTB32 KO CART cells also had similar antigen (CD19)-dependent proliferation and cancer cell killing as compared to wt CART cells in vitro (FIGS. 3A and 3B). ZBTB32 KO CART cells produced higher levels of pro-inflammatory cytokines IFNg, IL2 and TNFalpha when co-cultured with cancer cells in vitro (FIG. 3C).

Next, in vivo studies were performed using TMD8 and TMD8 Luciferase tumor models to evaluate the in vivo efficacy of wt and ZBTB32 KO CART-mediated anti-tumor immune responses. For the TMD8 model, 1×10⁶ CAR+ cells per mouse were transplanted and for the TMD8 luciferase tumor model, 0.4×10⁶ CAR+ cells/mouse and 0.4×10⁶ CAR+ cells/mouse were transplanted. The data from these experiments is shown in FIGS. 4-7.

For the first experiment in the TMD8 model, GFP wt (non-CAR), wt or ZBTB32 KO CART cells were injected at the dose of 1×106 CAR+ cells per mouse on day 10. As shown in FIGS. 4A-4B, animals treated with ZBTB32 KO CART cells (gRNA 6 or gRNA 7) had lower tumor volume at earlier time points (between time points day 25 to day 39) as compared to animals treated with wt CART cells. The data demonstrates that ZBTB32 KO CART cells have more rapid tumor control as compared to wt CART cells.

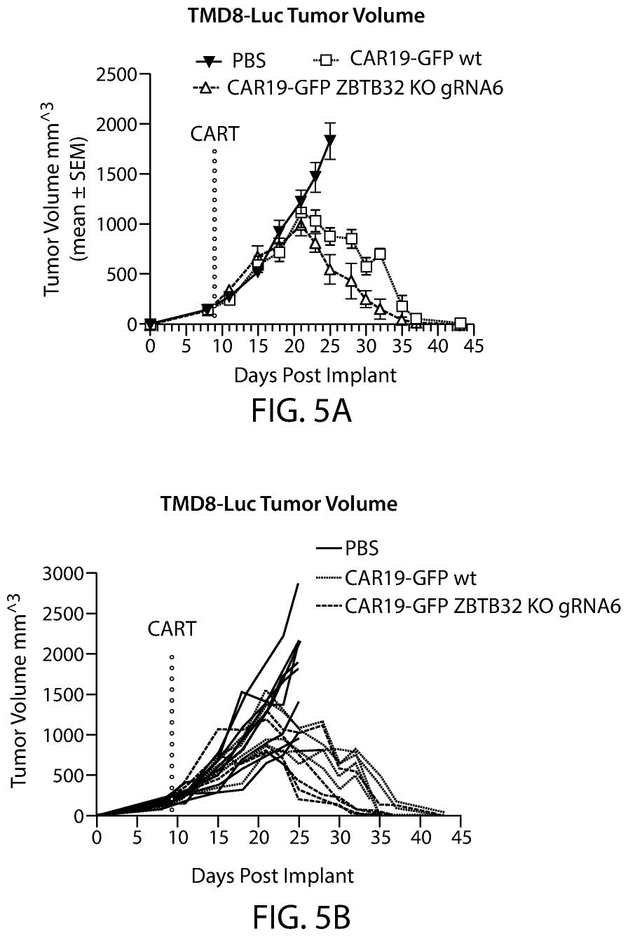

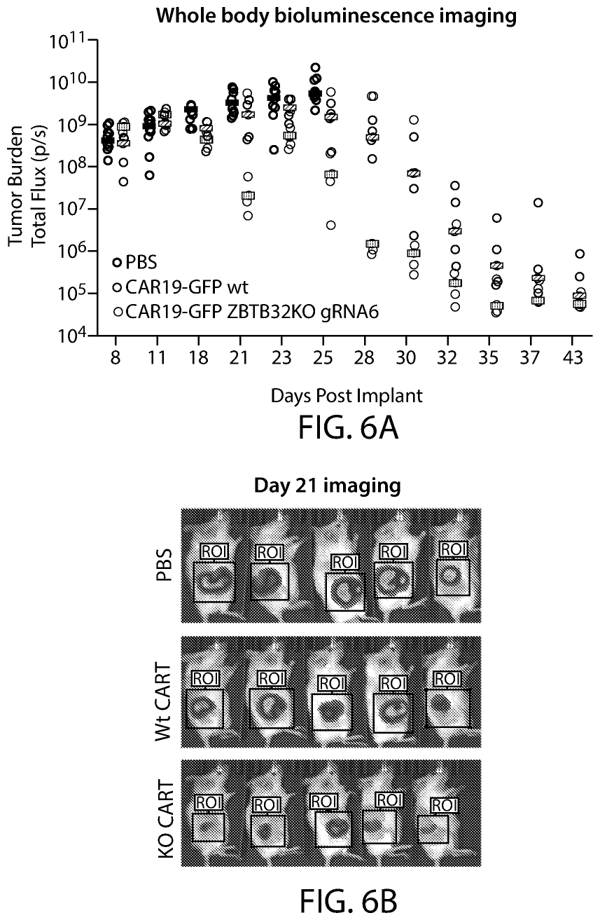

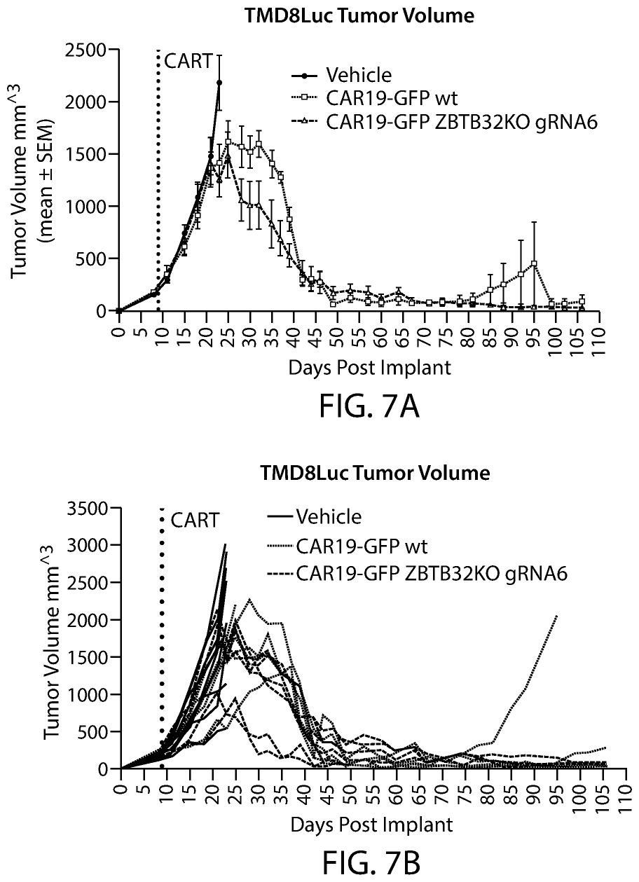

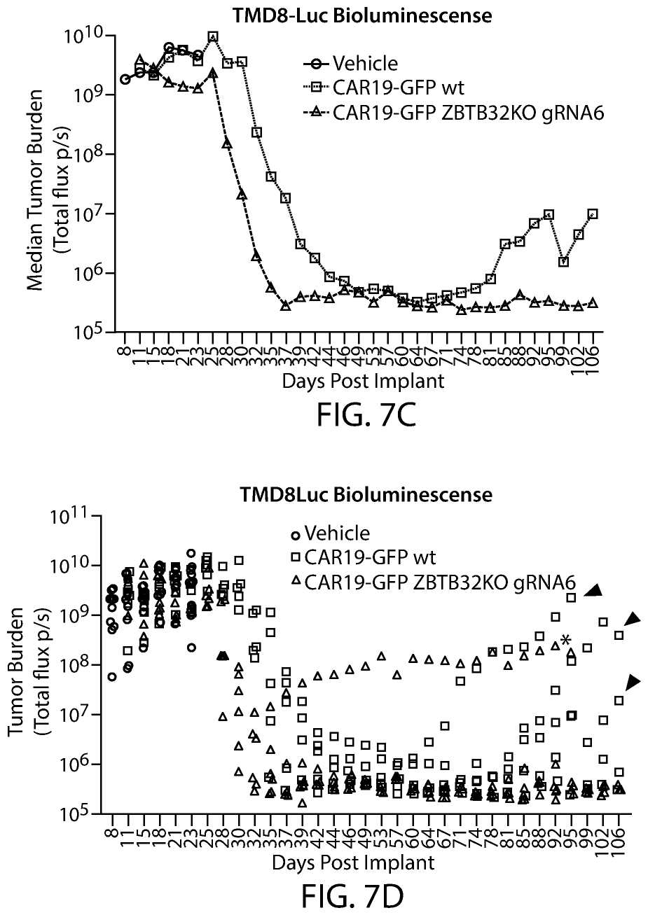

In the next experiment with the TMD8-Luc model, wt or ZBTB32 KO CART cells were injected at the dose of 2×106 CAR+ cells per mouse on day 9. As shown in FIGS. 5A-5B, tumor bearing animals treated with ZBTB32 KO CART cells had lower tumor volume as compared to animals treated with wt CART cells. The data demonstrates more rapid tumor control by ZBTB32 KO CART cells than wt CART cells. Bioluminescence imaging of animals bearing TMD8-Luc tumors treated with ZBTB32 KO CART cells showed more rapid tumor control by ZBTB32 KO CART cells than wt CART cells (FIG. 6A) and significantly lower bioluminescence on day 21 in the ZBTB32 KO CART cells treated mice than wt CART cells treated mice (FIG. 6B). Similarly, when tumor bearing mice were treated with 0.4×10⁶ CAR+ cells per mouse, more rapid tumor control by ZBTB32 KO CART cells than wt CART cells was observed (FIGS. 7A-7D). In addition, 3 out of 5 wt CART cells treated mice showed tumor relapse, while no ZBTB32 KO CART cells treated mice showed tumor relapse, highlighting better persistence of immune protection by ZBTB32 KO CART cells (FIGS. 7A-7D). The data from these experiments consistently shows that ZBTB32 KO CART cells are more potent in killing tumor cells, eliminate tumor cells more rapidly and have better long-term immune protection to prevent tumor relapse compared to wt CART cells in vivo (FIGS. 4-7).

T cell state is dynamically controlled by a set of transcription factors. TCF7 is a key transcription factor critical for the stem cell memory-like T cell (Tscm) state. It has been shown that TCF7⁺ CD8⁺ T cell frequency within tumor predicts response and better patient survival after PD-1 blockade in many human cancers. Transcription factor Eomes expression is low in Tscm cells and increased in exhausted or terminally differentiated CD8⁺ T cells. Recent studies identified TOX as a transcription factor epigenetically reprograming CD8 T cells to drive T cell exhaustion during chronic virus infection and cancer.

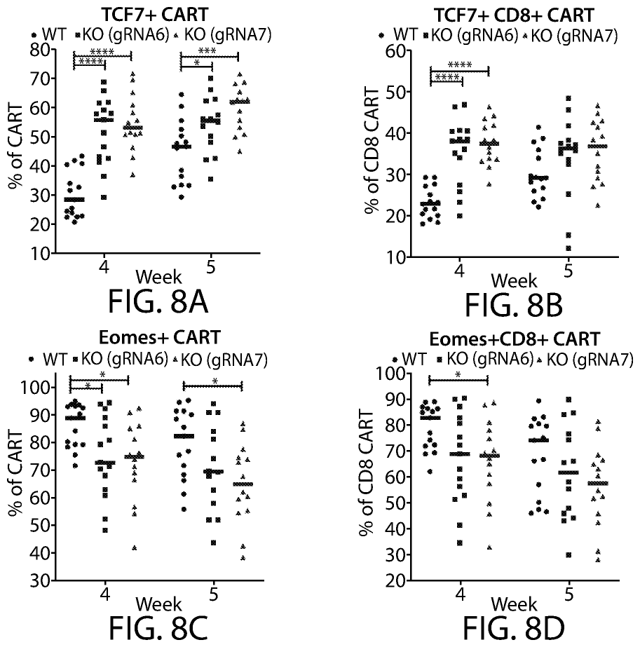

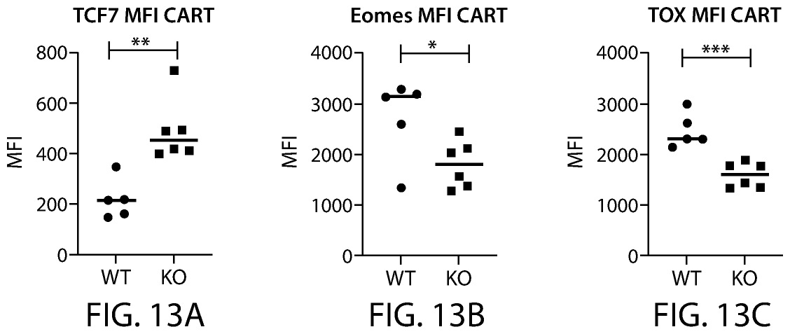

To investigate the relationship between ZBTB32 and T cell state, the effect of ZBTB32 knockout on the expression of T cell transcription factors in CART cells was evaluated. Flow cytometry analyses showed that ZBTB32 KO CART cells had higher levels of TCF7 and reduced levels of Eomes (FIGS. 8A-8D and FIGS. 13A-13B). The level of TOX was also lower in ZBTB32 KO CART cells (FIG. 13C). This data suggests that ZBTB32 regulates T cell state during anti-tumor immune responses in vivo.

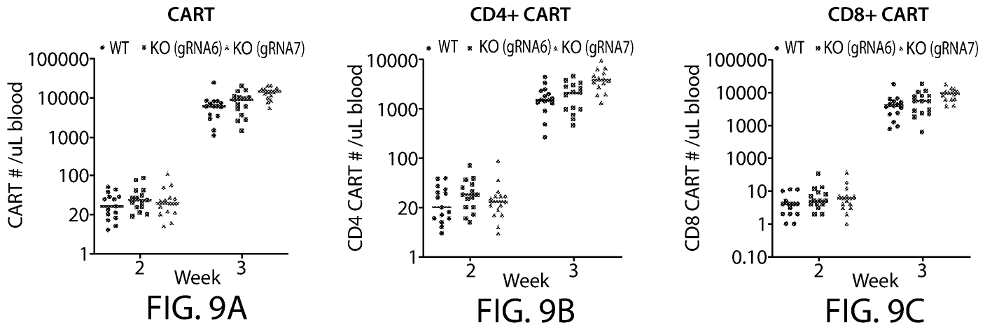

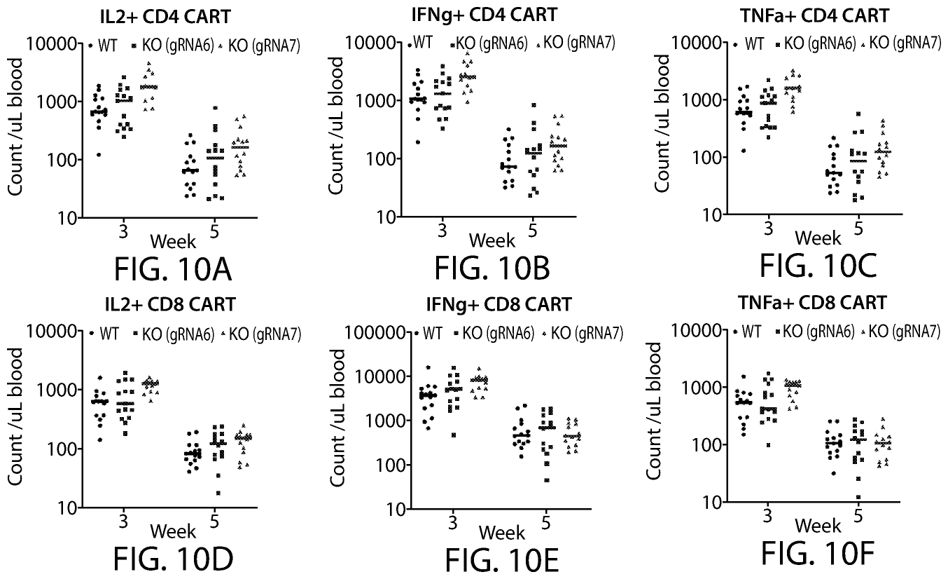

Compared to wt CART cells, there were more total ZBTB32KO CART cells (CD4+ and CD8+ T cells) in the blood (FIGS. 9A-9C). A higher number of IL2, IFNg and TNFalpha cytokine producing ZBTB32KO CART cells in both the CD4+ T cell population (FIGS. 10A-10C) and CD8+ T cell population (FIGS. 10D-10F) was also observed

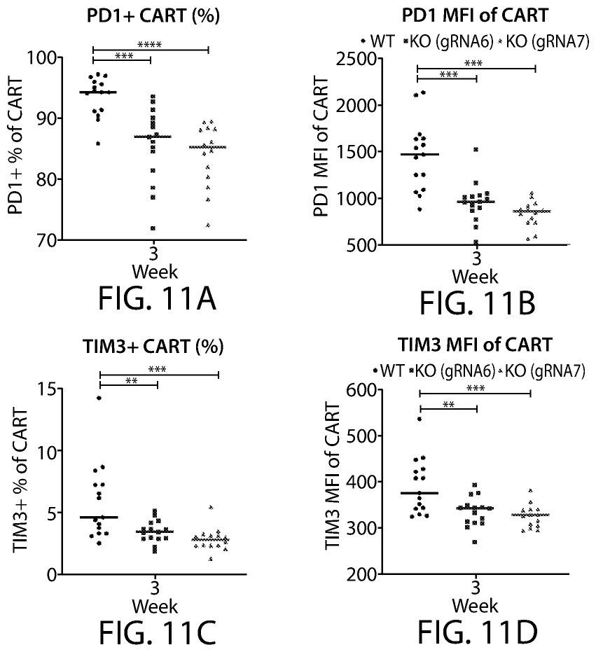

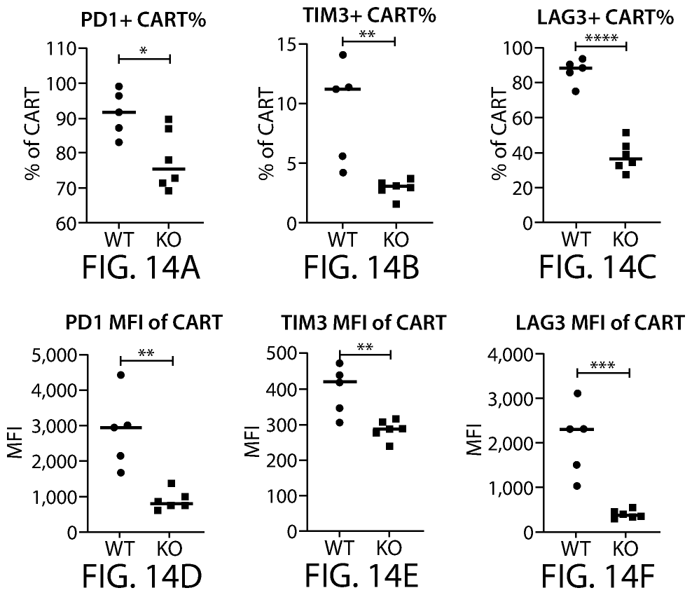

Importantly, ZBTB32 KO CART cells had significantly lower levels of exhaustion markers such as PD1, TIM3 and LAG3 as compared to wt CART cells (FIGS. 11A-11D and FIGS. 14A-14F). This data suggests that ZBTB32 KO CART cells are more resistant to exhaustion in vivo. In addition, when tumors were cleared in mice, a 3-5 fold higher number of ZBTB32KO CART cells were observed in the spleens of mice compared to the numbers of wt CART cells (FIG. 12A). Increase in total number of cells was also observed for both CD4+ CART cells and CD8+ CART cells in the spleen of mice (FIG. 12B-12C). This increase in the number of ZBTB32KO CART cells in mice after tumor clearance, indicates enhanced persistence of ZBTB32KO CART cells in vivo.

### SUMMARY

ZBTB32 knockout in CART cells enhances CART cell expansion, cytokine production, persistence, resistance to exhaustion and ultimately more potent and better long-term anti-tumor activity in vivo. This beneficial effect in CART cells could also be applicable for solid tumors by ZBTB32 inhibition alone or in combination with targeted or other 10 therapies given the regulation of ZBTB32 on key T cell transcription factors, exhaustion genes and T cell state.

### Example 3: Effect of ZBTB32 Knockout on Cancer Cells

This Example describes the effect of ZBTB32 knockout on the growth of cancer cells.

TMD8-cas9 stable cells were made by Cas9-containing lentivirus transduction and selected with 20 ug/ml Blasticidin. TMD8-cas9 cells were then infected with lentivirus containing non-targeting gRNA (gRNA NT, GTAGCGAACGTGTCCGGCGT (SEQ ID NO: 3014)) or ZBTB32 gRNA6 (CCAGCCGATCAGAGCCATAG (SEQ ID NO: 3004)) and selected with 0.5 ug/ml Puromycin.

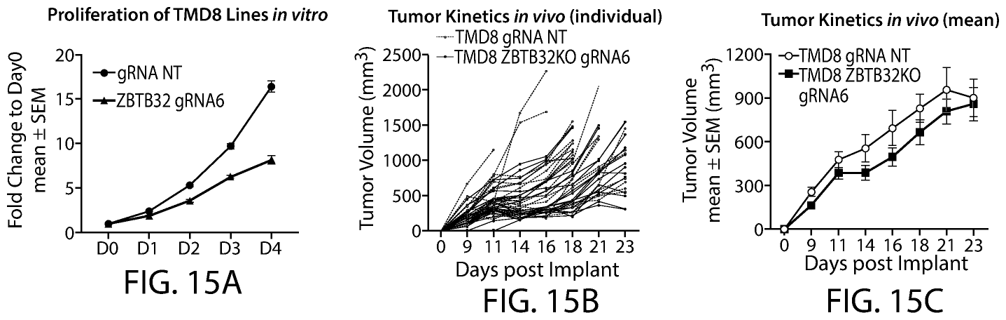

To measure the cell growth kinetics in vitro, TMD8 gRNA NT or TMD8 ZBTB32 gRNA6 stable cells were seeded into 384-well plate and cell growth was determined using CellTiter-Glo luminescent cell viability kit at days 0, 1, 2, 3 and 4. As shown in FIG. 15A, knockout of ZBTB32 in TMD8 cells resulted in reduced cell proliferation in vitro compared to control cells.

To determine the tumor growth kinetics in vivo, TMD8 gRNA NT and TMD8 ZBTB32 gRNA6 cells were re-suspended in a 1:1 ratio of PBS without CaCl₂ or MgCl₂ and matrigel. After collecting and spinning at 1500 rpm for 5 minutes, cells were re-suspended to a concentration of 5×10⁶ cells per 100 μl for implantation. Cells were implanted subcutaneously in the right flank of NSG mice at Day 0 (5×10⁶ cells per mouse, 20 mice per group). Tumors were monitored via caliper measurements 2-3 times weekly. Mice were terminated as tumor volumes neared endpoint (>1000 mm³). At the end point (Day 23), there were 7 and 12 mice left in control (gRNA NT) and ZBTB32 KO TMD8 tumor groups, respectively.

Control (gRNA NT) TMD8 tumors consistently grew slightly faster than ZBTB32KO tumors. Slight decrease in mean tumor volume in control group on Day 23 can be attributed to the fact that several mice with tumors neared endpoint (>1000 mm³) had been sacrificed at the previous measurement on Day 21. FIGS. 15B-15C show that ZBTB32 KO TMD8 tumors consistently demonstrated delayed growth kinetics in vivo over time.

### EQUIVALENTS

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A cell expressing a chimeric antigen receptor (CAR),
wherein the CAR comprises an antigen-binding domain, a transmembrane domain, and an intracellular signaling domain, and
wherein the cell has reduced expression and/or a reduced biological activity of ZBTB32.

2. The cell of claim 1, wherein:
a) the cell has no detectable expression and/or biological activity of ZBTB32;
(b) the cell has reduced expression of ZBTB32;
(c) the level of ZBTB32 protein is reduced;
(d) the stability of ZBTB32 protein is reduced;
(e) the level of ZBTB32 mRNA is reduced;
(f) the stability of ZBTB32 mRNA is reduced;
(g) the cell has reduced ZBTB32 transcription;
(h) the cell has reduced ZBTB32 translation;
(i) the ZBTB32 genomic locus is altered, optionally wherein:
(1) the ZBTB32 gene comprises a deletion or insertion; and/or
(2) the ZBTB32 gene comprises an epigenomic modification;
(j) the cell has a reduced biological activity of ZBTB32;
(k) a transcription repressor function of ZBTB32 is reduced; or
(l) the interaction between ZBTB32 and one or more binding partners is reduced, optionally wherein the one or more binding partners comprise Fanconi anemia complementation group C (FANCC), thioredoxin interacting protein (TXNIP), Vitamin D3 upregulated protein 1 (VDUP1), Zinc finger and BTB domain-containing protein 16 (Zbtb16), Zinc-finger elbow-related proline domain protein 2 (Zpo2), GATA binding protein 3 (Gata3), GATA binding protein 2 (Gata2), or B-cell lymphoma 6 (Bcl-6).

3. The cell of claim 1, wherein the cell comprises a ZBTB32 inhibitor, or the cell has been contacted with, or is being contacted with, a ZBTB32 inhibitor, optionally wherein:
(a) the ZBTB32 inhibitor comprises a small molecule; or
(b) the ZBTB32 inhibitor comprises:
(1) a gene editing system targeting the ZBTB32 gene or one or more components thereof;
(2) a nucleic acid encoding one or more components of the gene editing system; or
(3) a combination of (1) and (2).

4.-5. (canceled)

6. The cell of claim 3, wherein the gene editing system:
(a) is a CRISPR/Cas system, a zinc finger nuclease system, a TALEN system, or a meganuclease system;
(b) binds to a target sequence in the ZBTB32 gene;
(c) binds to a target sequence in an early exon or intron of the ZBTB32 gene;
(d) binds to a target sequence of the ZBTB32 gene, and the target sequence is upstream of exon 4;
(e) binds to a target sequence in a late exon or intron of the ZBTB32 gene;
(f) binds to a target sequence that is downstream of a preantepenultimate exon of the ZBTB32 gene;
(g) binds to a target sequence that comprises a splice junction of the ZBTB32 gene;
(h) binds to a target sequence in a coding region of the ZBTB32 gene;
(i) binds to a target sequence in a non-coding region of the ZBTB32 gene;
(j) binds to a target sequence in a regulatory element of the ZBTB32 gene; or
(k) is a CRISPR/Cas system comprising a guide RNA (gRNA) molecule comprising a targeting sequence which hybridizes to a target sequence of the ZBTB32 gene.

7.-16. (canceled)

17. The cell of claim 3, wherein the ZBTB32 inhibitor comprises:
(a) a small interfering RNA (siRNA) or a small hairpin RNA (shRNA) targeting the ZBTB32 gene, or a nucleic acid encoding the siRNA or shRNA, optionally wherein the siRNA or shRNA comprises a nucleotide sequence complementary to a sequence of an mRNA of the ZBTB32 gene;
(b) an antisense oligonucleotide (ASO) targeting the ZBTB32 gene, or a nucleic acid encoding the ASO, optionally wherein the ASO comprises a nucleotide sequence complementary to a sequence of an mRNA of the ZBTB32 gene;
(c) a protein;
(d) a dominant negative variant of a protein encoded by the ZBTB32 gene, or a nucleic acid encoding the dominant negative variant;
(e) a dominant negative binding partner of a protein encoded by the ZBTB32 gene, or a nucleic acid encoding the dominant negative binding partner; or
(f) an antibody molecule which binds to a protein encoded by the ZBTB32 gene, or a nucleic acid encoding the antibody molecule, optionally wherein the antibody molecule comprises a single-domain antibody (sdAb) or nanobody that binds to a protein encoded by the ZBTB32 gene.

18.-39. (canceled)

40. The cell of claim 1, wherein the cell:
(a) has an enhanced T cell-mediated anti-tumor response;
(b) has increased proliferation and/or cytokine production;
(c) has an altered T cell state;
(d) has enhanced resistance to exhaustion and enhanced long-term immune protection in vivo;
(e) has an increased expression of MHCII or MHCII transactivator CIITA;
(f) expands at a higher rate in vivo compared to a reference cell;
(g) has an improved immunological memory phenotype;
(h) is a human cell; or
(i) has reduced expression and/or a reduced biological activity of Tet2 or IKZF2.

41.-46. (canceled)

47. The cell of claim 1, wherein the cell is an immune effector cell or a population of immune effector cells,
optionally wherein the immune effector cell is a T cell, a B cell, or an NK cell, optionally wherein the T cell is:
(a) an alpha beta T cell (aβ T cell);
(b) a CD4+ T cell, a CD8+ T cell, or a combination thereof;
(c) a CD8+ T cell or regulatory T cell (Treg);
(d) a gamma delta T cell (γδ T cell); or
(e) a chimeric antigen receptor T (CART) cell.

48.-59. (canceled)

60. The cell of claim 1, wherein:
(a) the antigen-binding domain binds to a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p⁵³ mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1;
(b) the intracellular signaling domain comprises a primary signaling domain and/or a costimulatory signaling domain, wherein the primary signaling domain comprises a functional signaling domain of a protein chosen from CD3 zeta, CD3 gamma, CD3 delta, CD3 epsilon, common FcR gamma (FCER1G), FcR beta (Fc Epsilon Rib), CD79a, CD79b, Fc gamma RIIa, DAP10, or DAP12; or
(c) the intracellular signaling domain comprises a costimulatory signaling domain, or a primary signaling domain and a costimulatory signaling domain, wherein the costimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

61.-75. (canceled)

76. A method of increasing the therapeutic efficacy of the CAR-expressing cell of claim 1, comprising:
reducing the expression and/or a biological activity of ZBTB32 in the cell,
thereby increasing the therapeutic efficacy of the CAR-expressing cell.

77. A method of increasing the therapeutic efficacy of the CAR-expressing cell of claim 1, comprising:
contacting the cell with a ZBTB32 inhibitor,
thereby increasing the therapeutic efficacy of the CAR-expressing cell optionally wherein the inhibitor is:
(a) a small molecule that reduces the expression and/or a biological activity of ZBTB32;
(b) a gene editing system targeting the ZBTB32 gene;
(c) a nucleic acid that inhibits expression of ZBTB32, optionally wherein the nucleic acid comprises an siRNA, shRNA, or ASO;
(d) a dominant negative variant of a protein encoded by the ZBTB32 gene, or a binding partner of a protein encoded by the ZBTB32 gene;
(e) an antibody molecule that binds to a protein encoded by the ZBTB32 gene, optionally wherein the antibody molecule comprises a single-domain antibody (sdAb) or nanobody;
(f) a nucleic acid encoding (b) or a component thereof or any of (c)-(d); or
(g) any combination of (a)-(f);
optionally wherein:
(i) the cell is contacted with the ZBTB32 inhibitor ex vivo;
(ii) the cell is contacted with the ZBTB32 inhibitor in vivo, optionally wherein the cell is contacted with the ZBTB32 inhibitor in vivo prior to delivery of a nucleic acid encoding a CAR into the cell or after the cells have been administered to a subject in need thereof;
(iii) the method further comprises contacting the cell with a Tet2 inhibitor or an IKZF2 inhibitor; or
(iv) the cell has been contacted with a Tet2 or an IKZF2 inhibitor.

78.-86. (canceled)

87. A method for treating a cancer in a subject, the method comprising administering to the subject an effective amount of the cell of claim 1.

88. (canceled)

89. The method of claim 87, wherein the cancer is:
(a) a hematological cancer, optionally wherein the cancer is a lymphoma, a myeloma, or a leukemia;
(b) a B cell malignancy, optionally wherein the B cell malignancy is a B cell lymphoma or leukemia;
(c) a lymphoma, optionally wherein the lymphoma is a non-Hodgkin's lymphoma or a diffuse large B-cell lymphoma (DLBCL);
(d) a myeloma, optionally wherein the myeloma is a multiple myeloma (MM);
(e) a leukemia, optionally wherein the leukemia is an acute lymphocytic leukemia (ALL) or a chronic lymphocytic leukemia (CLL);
(d) a solid tumor, optionally wherein the solid tumor is associated with immune cell infiltration;
(e) a chronic lymphocytic leukemia (CLL), acute myeloid leukemia (AML), acute lymphoblastic leukemia (ALL), B-cell acute lymphoblastic leukemia (B-ALL), T-cell acute lymphoblastic leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, a malignant lymphoproliferative condition, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenström macroglobulinemia, or pre-leukemia: or
(f) a colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, an environmentally induced cancer, or a metastatic lesion thereof.

90.-96. (canceled)

97. The method of claim 87, wherein:
(a) the cancer expresses a higher level of ZBTB32;
(b) the method further comprises administering to the subject a second therapeutic agent or modality; or
(c) the method further comprises administering to the subject a ZBTB32 inhibitor.

98.-99. (canceled)

100. A method of treating a subject in need thereof, the method comprising administering a CAR-expressing cell therapy comprising the cell of claim 1, in combination with a ZBTB32 inhibitor, optionally wherein:
(a) the subject receives a pre-treatment of the ZBTB32 inhibitor prior to the initiation of the CAR-expressing cell therapy;
(b) the subject receives concurrent treatment with the ZBTB32 inhibitor and the CAR expressing cell therapy;
(c) the subject receives treatment with the ZBTB32 inhibitor post-CAR-expressing cell therapy;
(d) the subject has a disease associated with expression of a tumor antigen, e.g., a proliferative disease, a precancerous condition, a cancer, or a non-cancer related indication associated with expression of the tumor antigen; or
(e) the method further comprises determining the expression or a biological activity of ZBTB32 in the cell.

101.-115. (canceled)

116. A method of treating a subject, the method comprising:
administering to the subject an effective amount of a ZBTB32 inhibitor,
thereby treating the subject,
wherein the subject has received, is receiving, or is about to receive therapy comprising the CAR-expressing cell of claim 1.

117. (canceled)

118. A method of manufacturing the CAR-expressing cell of claim 1, comprising:
introducing a nucleic acid encoding a CAR into a cell such that said nucleic acid (or CAR-encoding portion thereof) integrates into the genome of the cell, such that the expression or a biological activity of ZBTB32 is reduced,
thereby manufacturing the CAR-expressing cell, optionally wherein:
(a) the nucleic acid integrates within the ZBTB32 gene; or
(b) the nucleic acid integrates within a gene other than the ZBTB32 gene.

119.-121. (canceled)

122. A method of manufacturing the CAR-expressing cell of claim 1, comprising:
contacting a CAR-expressing cell ex vivo with a ZBTB32 inhibitor, thereby manufacturing the CAR-expressing cell,
optionally wherein the CAR-expressing cell has an improved property compared to the same CAR-expressing cell that has not been contacted ex vivo with the ZBTB32 inhibitor;
optionally wherein the improved property comprises an enhanced T cell-mediated anti-tumor response, an increased proliferation and/or cytokine production, a reduced T cell exhaustion, an enhanced resistance to exhaustion and enhanced long-term immune protection in vivo, an increased expression of MHCII and/or MHCII transactivator CIITA, a higher expansion rate in vivo, an improved immunological memory phenotype, or any combination thereof.

123.-125. (canceled)

126. A vector comprising a nucleotide sequence encoding the CAR expressed by the cell of claim 1 and a nucleotide sequence encoding a ZBTB32 inhibitor, optionally wherein:
(1) the inhibitor is:
(a) a gene editing system targeting the ZBTB32 gene;
(b) a nucleic acid that inhibits expression of ZBTB32, optionally wherein the nucleic acid comprises an siRNA, shRNA, or ASO;
(c) a dominant negative variant of a protein encoded by the ZBTB32 gene, or a binding partner of a protein encoded by the ZBTB32 gene;
(d) an antibody molecule that binds to a protein encoded by the ZBTB32 gene, optionally wherein the antibody molecule comprises a single-domain antibody (sdAb) or nanobody; or
(e) any combination of (a)-(d); or
(2) the nucleotide sequence encoding the CAR and the nucleotide sequence encoding the inhibitor are separated by a 2A site.

127.-128. (canceled)

129. A composition for ex vivo manufacture of the CAR-expressing cell of claim 1, comprising a ZBTB32 inhibitor, optionally wherein the inhibitor is:
(a) a small molecule that reduces the expression and/or a biological activity of ZBTB32;
(b) a gene editing system targeting the ZBTB32 gene;
(c) a nucleic acid that inhibits expression of ZBTB32, optionally wherein the nucleic acid comprises an siRNA, shRNA, or ASO;
(d) a dominant negative variant of a protein encoded by the ZBTB32 gene, or a binding partner of a protein encoded by the ZBTB32 gene;
(e) an antibody molecule that binds to a protein encoded by the ZBTB32 gene, optionally wherein the antibody molecule comprises a single-domain antibody (sdAb) or nanobody;
(f) a nucleic acid encoding (b) or a component thereof, (c), or (d); or
(g) any combination of (a)-(f).

130. (canceled)

131. A population of cells comprising one or more of the cell of claim 1, wherein;
(a) the population of cells comprises a higher percentage of cells having a phenotype or expressing a marker associated with a central memory T (T_{CM}) cell or a stem memory T (T_{SCM}) cell) than a reference population of cells; wherein:
(b) the percentage of cytokine producing cells in the population is at least 50% higher than that of a reference population of cells, wherein the reference population of cells is:
(i) a population of cells which does not comprise one or more cells in which the expression of ZBTB32 in the cell has been reduced; or
(ii) a population of cells which does not comprise one or more cells in which the biological activity of ZBTB32 in the cell has been reduced; or
(c) at least 50% of the population of cells have a phenotype or express a marker associated with a central memory T (T_{CM}) cell or a stem memory T (T_{SCM}) cell.

132.-135. (canceled)

136. A method of treating a cancer in a subject, comprising:
administering to the subject an effective amount of a ZBTB32 inhibitor and a second therapeutic agent or modality,
thereby threating the cancer in the subject, optionally wherein:
(i) the ZBTB32 inhibitor is administered prior to, concurrently with, or post administration of the second therapeutic agent or modality;
(ii) the ZBTB32 inhibitor comprises a small molecule;
(iii) the ZBTB32 inhibitor comprises:
(1) a gene editing system targeting the ZBTB32 gene or one or more components thereof;
(2) a nucleic acid encoding one or more components of the gene editing system; or
(3) a combination of (1) and (2).

137.-206. (canceled)

207. A method of increasing the efficacy of a therapeutic agent or modality, comprising:
administering to the subject an effective amount of a ZBTB32 inhibitor,
thereby increasing the efficacy of the therapeutic agent of modality, optionally wherein:
(a) the subject has a cancer; or
(b) the therapeutic agent or modality comprises an immunotherapy or a cell therapy.

208.-210. (canceled)

211. A method of increasing an immune response in a subject, comprising:
administering to the subject an effective amount of a ZBTB32 inhibitor,
thereby increasing the immune response in the subject,
optionally wherein:
(a) the subject has a cancer; or
(b) the subject has received, or is receiving, a second therapeutic agent or modality.

212.-214. (canceled)

215. A method of inhibiting the expression or a biological activity of ZBTB32, comprising:
contacting a cell with a ZBTB32 inhibitor; and
optionally further contacting the cell with a second therapeutic agent or modality;
thereby treating the cell,
optionally wherein the ZBTB32 inhibitor is contacted with the cell in vitro, ex vivo, or in vivo.

216. (canceled)

217. A gene editing system targeting the ZBTB32 gene, optionally wherein the gene editing system is a CRISPR/Cas gene editing system, a zinc finger nuclease system, a TALEN system, or a meganuclease system.

218.-222. (canceled)
